(19) United States
(12) Patent Application Publication
(10) Pub. No.: US 2026/0211320 A1
(43) Pub. Date: Jul. 23, 2026

(54) RESIST COMPOSITION, METHOD FOR PRODUCING RESIST PATTERN, SALT, AND RESIN

(71) Applicant: SUMIMOTO CHEMICAL COMPANY, LIMITED, Tokyo (JP)

(72) Inventors: Shohei TERAHIGASHI, Osaka (JP); Wataru NOJO, Osaka (JP); Koji ICHIKAWA, Osaka (JP)

(73) Assignee: SUMIMOTO CHEMICAL COMPANY, LIMITED, Tokyo (JP)

(21) Appl. No.: 19/448,788

(22) Filed: Jan. 14, 2026

(30) Foreign Application Priority Data
Jan. 17, 2025 (JP) 2025-007192
Aug. 7, 2025 (JP) 2025-132649

(51) Int. Cl. G03F 7/004; G03F 7/16; G03F 7/38

(52) U.S. Cl. CPC G03F 7/0045; G03F 7/168; G03F 7/38

(57) ABSTRACT

A resist composition includes a salt represented by formula (I) or a resin that includes a structural unit represented by formula (IP):

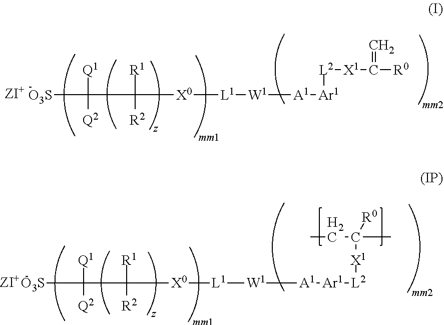

wherein Q¹, Q², R¹ and R² are each a hydrogen atom, a fluorine atom, or the like; z is an integer of 0 to 6; X⁰ is —O—, —CO—, —S—, or the like; mm1 is 0 or 1; L¹ and L² each represent a single bond or a substituted/unsubstituted hydrocarbon group; W¹ represents a substituted/unsubstituted alicyclic hydrocarbon group; A¹ represents a substituted/unsubstituted hydrocarbon group; Ar¹ represents an aromatic hydrocarbon group having an iodine atom, and the aforementioned group optionally has a substituent other than the iodine atom; X¹ represents a single bond, —O—, —CO—, or the like; R⁰ represents a hydrogen atom, a halogen atom, or an alkyl group optionally having a halogen atom; and mm2 represents an integer of 1 to 5: ZI⁺ represents an organic cation.

### TECHNICAL FIELD

The present invention relates to a resist composition, a method for producing a resist pattern, a salt, and a resin.

### BACKGROUND ART

Patent Literature 1 discloses an anion represented by the following formula.

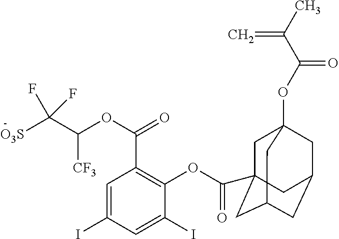

### CITATION LIST

### Patent Literature

Patent Literature 1: Japanese Patent Laid-Open No. 2024-109570

### SUMMARY OF INVENTION

### Problems to be Solved by Invention

The objective is to provide a resist composition that forms a resist pattern with good CD uniformity (CDU) compared to a resist pattern formed from a resist composition that contains a salt containing the above anion.

### Means to Solve the Problems

The present invention includes the following inventions.
[1] A resist composition including a salt represented by formula (I) or a resin that includes a structural unit represented by formula (IP):

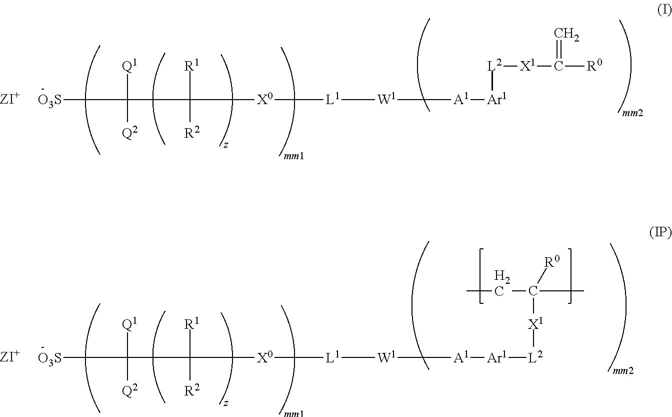

wherein, in formula (I) and formula (IP),
Q¹, Q², R¹, and R² each independently represent a hydrogen atom, a fluorine atom, an perfluoroalkyl group having 1 to 6 carbon atoms, or an alkyl group having 1 to 6 carbon atoms;
z represents an integer of 0 to 6, and when z is 2 or larger, a plurality of R¹ and R² may be the same or different from each other;
X⁰ represents a single bond, —O—, —CO—, —S—, —SO—, —SO₂—, —NR⁷—, or a combined group of these groups;
R⁷ represents a hydrogen atom or an alkyl group having 1 to 6 carbon atoms;
mm1 represents 0 or 1;
L¹ and L² each independently represent a single bond, or a hydrocarbon group having 1 to 28 carbon atoms and optionally having a substituent, and —CH₂— contained in the hydrocarbon group is optionally replaced with —O—, —S—, —SO—, —SO₂—, —NR⁷—, or —CO—;
W¹ represents an alicyclic hydrocarbon group having 3 to 36 carbon atoms and optionally having a substituent, and —CH₂— contained in the alicyclic hydrocarbon group is optionally replaced with —O—, —CO—, —S—, —SO—, —SO₂—, or —NR⁷—;
A¹ represents a hydrocarbon group having 1 to 20 carbon atoms and optionally having a substituent, and —CH₂— contained in the hydrocarbon group is optionally replaced with —O—, —S—, —SO—, —SO₂—, —NR⁷—, or —CO—;
Ar¹ represents an aromatic hydrocarbon group having 6 to 18 carbon atoms having an iodine atom, and the aromatic hydrocarbon group may have a substituent other than an iodine atom;
X¹ represents a single bond, —O—, —CO—, —NR⁷—, —S—, —SO—, —SO₂—, a phenylene group optionally having a substituent, or a combined group of these groups;
R⁰ represents a hydrogen atom, a halogen atom, or an alkyl group having 1 to 6 carbon atoms and optionally having a halogen atom;
mm2 represents an integer of 1 to 5, and when mm2 is 2 or larger, a plurality of groups in parentheses may be the same or different from each other; and
ZI⁺ represents an organic cation.
[2] The resist composition according to [1], wherein L¹ is a single bond, a chain hydrocarbon group having 1 to 10 carbon atoms and optionally having a substituent, where —CH₂— contained in the chain hydrocarbon group is optionally replaced with —O—, —CO—, —S—, —SO—, —SO₂—, or —NR⁷—, a cyclic hydrocarbon group having 3 to 18 carbon atoms and optionally having a substituent, where —CH₂— contained in the cyclic hydrocarbon group is optionally replaced with —O—, —CO—, —S—, —SO—, —SO₂—, or —NR⁷—, or a combined group of these groups.
[3] The resist composition according to [1] or [2], wherein W¹ is an alicyclic hydrocarbon group having 5 to 18 carbon atoms and optionally having a substituent.
[4] The resist composition according to any one of [1] to [3],
wherein A¹ is ***—X⁰¹L⁰¹- or ***-L⁰¹-X⁰¹—;
X⁰¹ represents —O—, —S—, —SO—, —SO₂—, —NR⁷—, or —CO—;
L⁰¹ represents a single bond, or a hydrocarbon group having 1 to 18 carbon atoms, where the hydrocarbon group optionally has a substituent, and —CH₂— contained in the hydrocarbon group is optionally replaced with —O—, —S—, —SO—, —SO₂—, —NR⁷—, or —CO—;
*** represents a bonding site to W¹.
[5] The resist composition according to [4], wherein X⁰¹ is —O— or —CO—, and L⁰¹ is a single bond or an alkanediyl group having 1 to 6 carbon atoms, where —CH₂— contained in the alkanediyl group is optionally replaced with —O— or —CO—.
[6] The resist composition according to any one of [1] to [5], wherein Ar¹ is an aromatic hydrocarbon group having 6 to 10 carbon atoms and having an iodine atom.
[7] The resist composition according to any one of [1] to [6], wherein X¹ is a single bond or a group represented by any of formula (X¹-1) to (X¹-10).

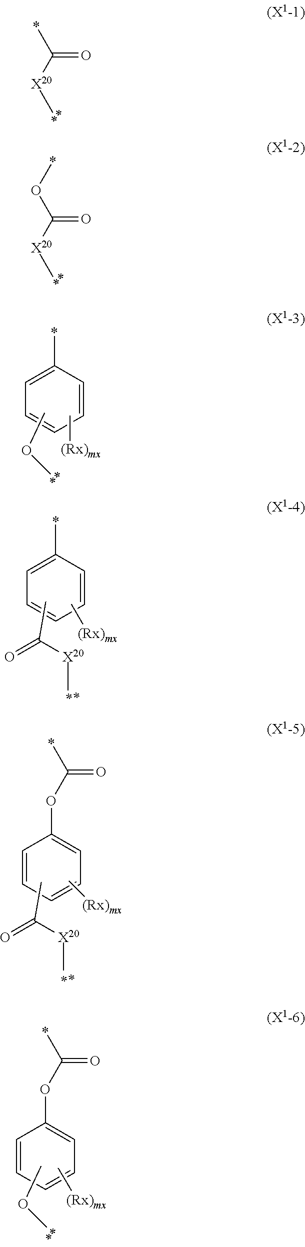

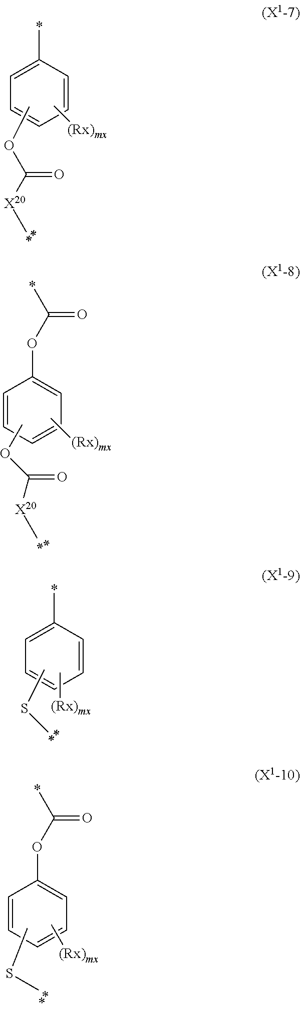

wherein in formula (X¹-1) to formula (X¹-10),
* and ** are bonding sites, and * represents a bonding site to a carbon atom bonded to R⁰;
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms;
mx represents an integer of 0 to 4; when mx is 2 or more, a plurality of Rx are optionally the same or different from each other;
X²⁰ represents —O— or —NR⁷—; and
R⁷ represents a hydrogen atom or an alkyl group having 1 to 6 carbon atoms.
[8] The resist composition according to any one of [1] to [7],
wherein the resist composition further includes a resin that includes a structural unit represented by formula (a2-A); or
the resin that includes a structural unit represented by the formula (IP) further includes a structural unit represented by formula (a2-A),

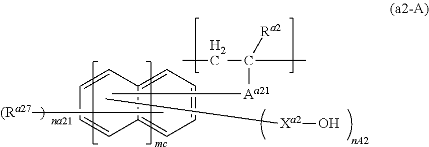

wherein in the formula (a2-A)
R^{a2} represents a hydrogen atom, a halogen atom, or an alkyl group having 1 to 6 carbon atoms and optionally having a halogen atom;
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group;
A^{a21} represents a single bond, or an alkanediyl group having 1 to 12 carbon atoms, and —CH₂— contained in the alkanediyl group is optionally replaced with —O—, —CO— or —NR^{a28}—.

R^{a28} represents a hydrogen atom, or an alkyl group having 1 to 6 carbon atoms;
X^{a2} represents a single bond or a carbonyl group;
nA2 represents an integer of 1 to 5, and when nA2 is 2 or larger, a plurality of groups in parentheses may be the same or different from each other;
na21 represents an integer of 0 to 4, and when na21 is 2 or larger, a plurality of R^{a27} may be the same or different from each other; and
mc represents an integer of 0 to 2;
[9] The resist composition according to any one of [1] to [8],
wherein the resist composition further includes a resin including a structural unit (a1) having an acid labile group; or
the resin that includes a structural unit represented by the formula (IP) further includes a structural unit (a1) having an acid labile group, and
the structural unit (a1) having an acid labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5), and a structural unit represented by formula (a1-6),

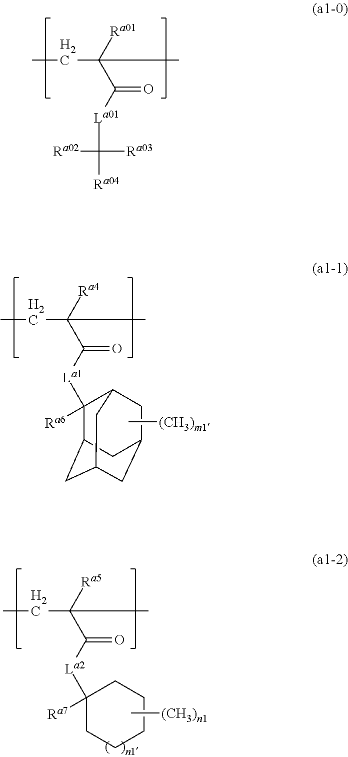

wherein, in formula (a1-0), formula (a1-1), and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—;
R^{a01}, R^{a4} and R^{a5} each independently represent a hydrogen atom, a halogen atom, or an alkyl group having 1 to 6 carbon atoms and optionally having a halogen atom;
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or a combined group of these groups, and the alkyl group, the alicyclic hydrocarbon group and the aromatic hydrocarbon group optionally have a halogen atom;
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or a group formed by combining these groups, and the alkyl group, the alkenyl group, the alicyclic hydrocarbon group, and the aromatic hydrocarbon group optionally have a halogen atom;
m1′ represents an integer of 0 to 14;
n1 represents an integer of 0 to 14; and
n1′ represents an integer of 0 to 3;

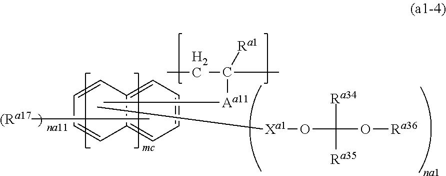

wherein in formula (a1-4),
R^{a1} represents a hydrogen atom, a halogen atom, or an alkyl group having 1 to 6 carbon atoms and optionally having a halogen atom;
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group;
A^{a11} represents a single bond or an alkanediyl group having 1 to 12 carbon atoms, and —CH₂— contained in the alkanediyl group is optionally replaced with —O—, —CO— or —NR^{a18}—.
R^{a18} represents a hydrogen atom or an alkyl group having 1 to 6 carbon atoms;
X^{a1} represents a single bond or a carbonyl group;
R^{a34} and R^{a35} each independently represent a hydrogen atom or a hydrocarbon group having 1 to 12 carbon atoms, R^{a36} represents a hydrocarbon group having 1 to 20 carbon atoms, or R^{a34} represents a hydrogen atom or a hydrocarbon group having 1 to 12 carbon atoms, R^{a35} and R^{a36} are bonded to each other to form a divalent heterocyclic group having 3 to 20 carbon atoms together with —C—O— to which they are bonded, and —CH₂— contained in the hydrocarbon group and the divalent heterocyclic group are optionally replaced with —O— or —S—;
na1 represents an integer of 1 to 5, and when na1 is 2 or larger, a plurality of groups in parentheses may be the same or different from each other;
na11 represents an integer of 0 to 4, and when na11 is 2 or larger, a plurality of R^{a17} may be the same or different from each other; and
mc represents an integer of 0 to 2;

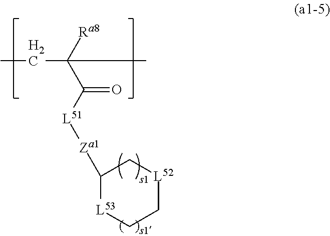

wherein, in formula (a1-5),
R^{a8} represents an alkyl group having 1 to 6 carbon atoms and optionally having a halogen atom, a hydrogen atom or a halogen atom;
Z^{a1} represents a single bond or *—(CH₂)ₕ₃—CO-L⁵⁴-, h3 represents an integer of 1 to 4, and * represents a bonding site to L⁵¹;
L⁵¹, L⁵², L⁵³ and L⁵⁴ each independently represent —O— or —S—;
s1 represents an integer of 1 to 3;
s1′ represents an integer of 0 to 3; and

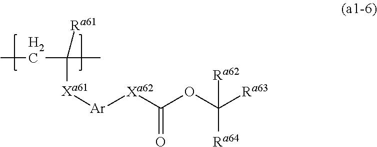

wherein, in formula (a1-6),
R^{a61} represents a hydrogen atom, a halogen atom, or an alkyl group having 1 to 6 carbon atoms and optionally having a halogen atom;
R^{a62}, R^{a63} and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with carbon atoms to which they are bonded, and R^{a64} represents an alkyl group having 1 to 6 carbon atoms or a cyclic hydrocarbon group having 3 to 18 carbon atoms and optionally having a substituent;
X^{a61} represents a single bond, —CO—O—*, or —CO—NR^{a65}—*, * represents a bonding site to Ar, and R^{a65} represents a hydrogen atom or an alkyl group having 1 to 6 carbon atoms;
X^{a62} represents a single bond, *—O-L^{a61}- or *—CO—O-L^{a62}-, * represents a bonding site to Ar, and L^{a65} and L^{a62} each independently represent an alkanediyl group having 1 to 4 carbon atoms; and
Ar represents an aromatic hydrocarbon group having 6 to 20 carbon atoms and optionally having a substituent.
[10] The resist composition according to any one of [1] to [9], further including a salt that generates an acid having a weaker acidity than an acid that is generated from the salt represented by the formula (I) or the resin that includes a structural unit represented by the formula (IP).
[11] A method for producing a resist pattern, including:
(1) a step of applying the resist composition according to any one of [1] to [10] onto a substrate;
(2) a step of drying the applied resist composition to form a composition layer;
(3) a step of exposing the composition layer;
(4) a step of heating the exposed composition layer; and
(5) a step of developing the composition layer after heating.
[12] A salt represented by the above formula (I).
[13] The salt according to [12], wherein L¹ is a single bond, a chain hydrocarbon group having 1 to 10 carbon atoms and optionally having a substituent, where —CH₂— contained in the chain hydrocarbon group is optionally replaced with —O—, —CO—, —S—, —SO—, —SO₂—, or —NR⁷—, a cyclic hydrocarbon group having 3 to 18 carbon atoms and optionally having a substituent, where —CH₂— contained in the cyclic hydrocarbon group is optionally replaced with —O—, —CO—, —S—, —SO—, —SO₂—, or —NR⁷—, or a combined group of these groups.
[14] The salt according to [12] or [13], wherein W¹ is an alicyclic hydrocarbon group having 5 to 18 carbon atoms and optionally having a substituent.
[15] The salt according to any of [12] to [14],
wherein A¹ is ***—X⁰¹-L⁰¹- or ***-L⁰¹-X⁰¹—;
X⁰¹ represents —O—, —S—, —SO—, —SO₂—, —NR⁷—, or —CO—;
L⁰¹ represents a single bond, or a hydrocarbon group having 1 to 18 carbon atoms, where the hydrocarbon group optionally has a substituent, and —CH₂— contained in the hydrocarbon group is optionally replaced with —O—, —S—, —SO—, —SO₂—, —NR⁷—, or —CO—;
*** represents a bonding site to W¹.
[16] The salt according to [15], wherein X⁰¹ is —O— or —CO—, and L⁰¹ is a single bond or an alkanediyl group having 1 to 6 carbon atoms, where —CH₂— contained in the alkanediyl group is optionally replaced with —O— or —CO—.
[17] The salt according to any one of [12] to [16], wherein Ar¹ is an aromatic hydrocarbon group having 6 to 10 carbon atoms and having an iodine atom.
[18] The salt according to any one of [12] to [17], wherein X¹ is a single bond or a group represented by any of the formula (X¹-1) to the (X¹-10) described above.
[19] A resin including a structural unit represented by the above formula (IP).

### Effects of Invention

By the use of the resist composition of the present invention, a resist pattern having good CD uniformity (CDU) can be produced.

### EMBODIMENTS FOR CARRYING OUT INVENTION

In the present specification, the “(meth)acrylic monomer” means “at least one of an acrylic monomer and a methacrylic monomer”. The descriptions of “(meth)acrylate”, “(meth)acrylic acid” and the like also represent the same meaning. In the groups described in the present specification, those that can have both a straight-chain structure and a branched structure may be any one of them. When —CH₂— contained in a hydrocarbon group or the like is replaced with —O—, —S—, —CO—, —SO—, —NR^{X}— (where X is any symbol) or —SO₂—, the same example applies to each group, and the number of carbon atoms before replacement is defined as the number of carbon atoms in the hydrocarbon group or the like. The term “combined group” means a group in which two or more of the exemplified groups are bonded, and the valences of these groups may be appropriately changed depending on the bonding form. The “derived” or “induced” means that a polymerizable C═C bond contained in the molecule becomes a —C—C— group (single bond) by polymerization. When stereoisomers exist, all stereoisomers are included. In each group, hydrogen atoms at any positions and in any number, which are contained in the group, are substituted with bonding sites, in some cases, depending on the number of substituents and the like. The number of carbon atoms in the substituent is not included in the number of carbon atoms in the substituted group. The acid labile group means a group that has a leaving group, causes detachment of the leaving group when the group comes into contact with an acid (for example, trifluoromethane sulfonic acid or the like), and forms a hydrophilic group such as a hydroxy group or a carboxy group. A base-labile group means a group that has a leaving group, causes detachment of the leaving group when the group comes in contact with a base (for example, trimethylamine, tetramethylammonium hydroxide or the like), and forms a hydrophilic group (for example, carboxy group, hydroxy group or the like).

In the present specification, the “solid content of the resist composition” means a sum of components excluding the solvent (E) which will be described later, from the total amount of the resist composition.

### [Salt Represented by Formula (I)]

The present invention relates to a salt represented by formula (I) (hereinafter referred to as “salt (I)” in some cases).

In the salt (I), a side having a negative charge is referred to as “anion (I)”, and a side having a positive charge is referred to as “cation (I)”, in some cases.

### (Anion (I))

In the formula (I), examples of the perfluoroalkyl groups of Q¹, Q², R¹ and R² include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group and a perfluorohexyl group. The number of carbon atoms of the perfluoroalkyl group is preferably 1 to 4, and is more preferably 1 to 3.

Examples of the alkyl group in Q¹, Q², R¹ and R² include a methyl group, an ethyl group, a propyl group, an isopropyl group, a butyl group, a sec-butyl group, a tert-butyl group, a pentyl group and a hexyl group. The number of carbon atoms of the alkyl group is preferably 1 to 4, and is more preferably 1 to 3.

At least one of Q¹ and Q² preferably contains a fluorine atom or a perfluoroalkyl group; at least one of Q¹ and Q² is more preferably a fluorine atom or a perfluoroalkyl group; Q¹ and Q² are each independently further preferably a trifluoromethyl group or a fluorine atom; and both of Q¹ and Q² are further more preferably fluorine atoms.

R¹ and R² are each independently preferably a hydrogen atom, a fluorine atom, a methyl group or a trifluoromethyl group, are each more preferably a hydrogen atom or a fluorine atom, and are each further preferably a hydrogen atom.

z is preferably an integer of 0 to 4, is more preferably an integer of 0 to 3, and is further preferably an integer of 0 to 2, and is further more preferably 0 or 1.

X⁰ is preferably a single bond, —O—, —CO—, —NR⁷—, or a combined group of these groups, and more preferably a single bond, *—CO—O—, *—O—CO—, *—O—, *—O—CO—O—, —NR⁷—, *—CO—NR⁷—, *—NR⁷—CO—, *—NR⁷—CO—, or *—O—CO—NR⁷—, and even more preferably *—CO—O—, *—O—CO—, *—O—, or *—O—CO—O—, and even still more preferably *—CO—O—, *—O—CO—, or *—O—CO—O— (where * represents a bonding site to C(R¹)(R²) or C(Q¹)(Q²))

Examples of the alkyl group of R⁷ include those similar to those exemplified by Q¹, Q², R¹, and R².

R⁷ is preferably a hydrogen atom or an alkyl group having 1 to 4 carbon atoms, more preferably a hydrogen atom or an alkyl group having 1 to 3 carbon atoms, further preferably a hydrogen atom, a methyl group, or an ethyl group, and still more preferably a hydrogen atom or a methyl group.

The hydrocarbon groups in L¹ and L² are divalent hydrocarbon groups, and examples thereof include a straight-chain or branched chain hydrocarbon group, and cyclic hydrocarbon groups such as a monocyclic or polycyclic (including a spiro ring, a condensed ring or bridged ring) alicyclic hydrocarbon group, a saturated or unsaturated alicyclic hydrocarbon group, and an aromatic hydrocarbon group, and may be the combined group of two or more of these groups (for example, a hydrocarbon group formed from an alicyclic hydrocarbon group or an aromatic hydrocarbon group and a chain hydrocarbon group). The chain hydrocarbon groups and the alicyclic hydrocarbon groups are also referred to as the aliphatic hydrocarbon groups, and the alicyclic hydrocarbon groups and the aromatic hydrocarbon groups are also referred to as the cyclic hydrocarbon groups. The number of carbon atoms of the hydrocarbon group is preferably 1 to 24, is more preferably 1 to 20, is further preferably 1 to 18, and is further more preferably 1 to 12.

Examples of the chain hydrocarbon group include an alkanediyl group, and examples thereof include: straight-chain alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; and
branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The terminal of the branched alkanediyl group may be an alkyl group such as a methyl group.

The number of carbon atoms of the chain hydrocarbon group may be 1 to 24, is preferably 1 to 18, is more preferably 1 to 12, is further preferably 1 to 10, is even more preferably 1 to 8, is far more preferably 1 to 6, and is still further preferably 1 to 4.

Examples of the monocyclic type or polycyclic type of divalent alicyclic hydrocarbon group include the following groups. The bonding site can be set to any position.

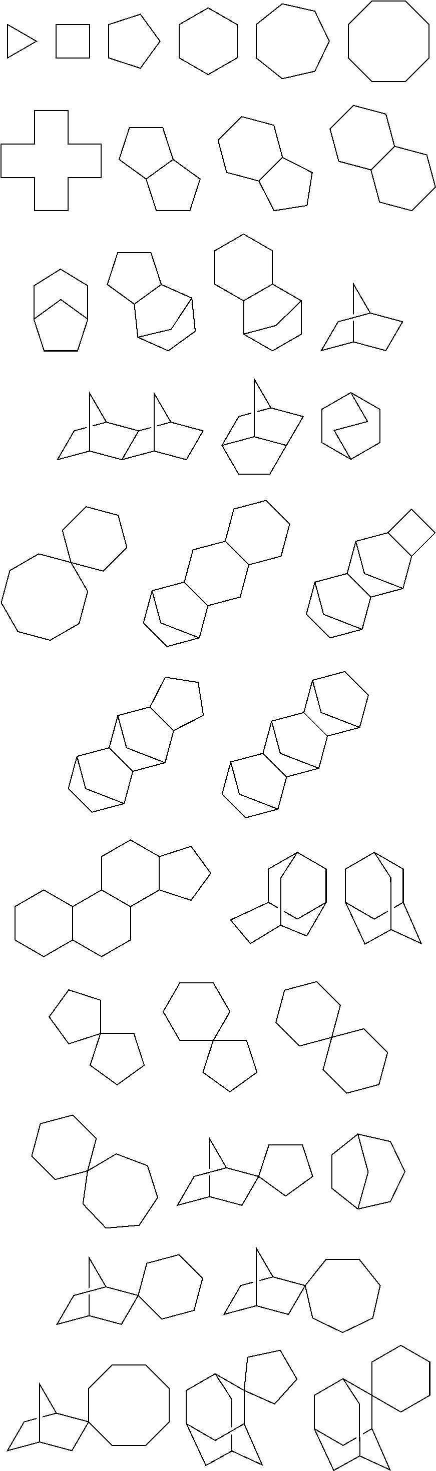

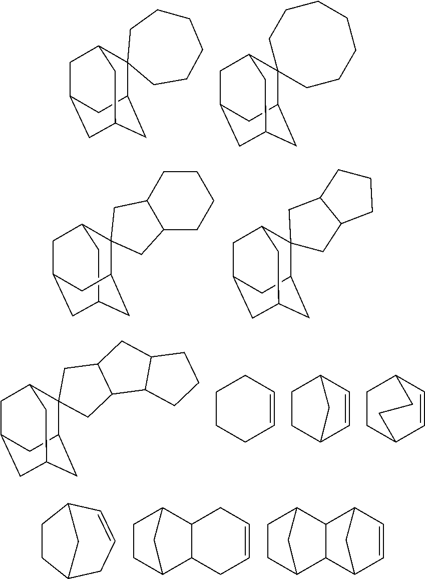

Specific examples thereof include: monocyclic type of divalent alicyclic hydrocarbon groups that are monocyclic cycloalkanediyl groups such as a cyclobutane-1,3-diyl group, a cyclopentane-1,3-diyl group, a cyclohexane-1,4-diyl group and a cyclooctane-1,5-diyl group; and
polycyclic type of divalent alicyclic hydrocarbon groups that are polycyclic cycloalkanediyl groups such as a norbornane-1,4-diyl group, a norbornane-2,5-diyl group, an adamantane-1,5-diyl group, an adamantane-2,6-diyl group, cycloalkyl groups such as spirocyclohexane-1,2′-cyclopentane group and spiroadamantane-2,3′-cyclopentane group, and a spiro ring having a cycloalkyl group bonded to each of a norbornyl group and an adamantyl group by spiro. The number of carbon atoms of the alicyclic hydrocarbon group may be 3 to 24, is preferably 3 to 18, is more preferably 3 to 16, and is further preferably 3 to 12.

Examples of the aromatic hydrocarbon groups include arylene groups such as a phenylene group, a naphthylene group, an anthrylene group, a biphenylene group and a phenanthrylene group. The number of carbon atoms of the aromatic hydrocarbon group may be 6 to 24, is preferably 6 to 18, is more preferably 6 to 14, and is further preferably 6 to 10.

Examples of the combined groups of two or more include a combined group of an alicyclic hydrocarbon group and a chain hydrocarbon group, a combined group of an aromatic hydrocarbon group and a chain hydrocarbon group, a combined group of an alicyclic hydrocarbon group and an aromatic hydrocarbon group, and a combined group of an alicyclic hydrocarbon group, an aromatic hydrocarbon group, and a chain hydrocarbon group. In the combination, two or more types of alicyclic hydrocarbon groups, aromatic hydrocarbon groups, and chain hydrocarbon groups may each be combined. In addition, any of the groups may be bonded to the X⁰ or the X¹.

Examples of the combined group of an alicyclic hydrocarbon group and a chain hydrocarbon group include -alicyclic hydrocarbon group-chain hydrocarbon group-, -chain hydrocarbon group-alicyclic hydrocarbon group-chain hydrocarbon group-, and -chain hydrocarbon group-alicyclic hydrocarbon group-.

Examples of the combined group of an aromatic hydrocarbon group and a chain hydrocarbon group include -aromatic hydrocarbon group-chain hydrocarbon group-, -chain hydrocarbon group-aromatic hydrocarbon group-chain hydrocarbon group-, and -chain hydrocarbon group-aromatic hydrocarbon group-.

Examples of the combined group of an alicyclic hydrocarbon group and an aromatic hydrocarbon group include -aromatic hydrocarbon group-alicyclic hydrocarbon group-, -alicyclic hydrocarbon group-aromatic hydrocarbon group-, and -alicyclic hydrocarbon group-aromatic hydrocarbon group-alicyclic hydrocarbon group-.

—CH₂— contained in the hydrocarbon group having 1 to 28 carbon atoms for L¹ and L² is optionally replaced with —O—, —CO—, —S—, —SO—, —SO₂—, or —NR⁷—.

When —CH₂— contained in hydrocarbon groups having 1 to 28 carbon atoms for L¹ and L² is replaced with —O—, —CO—, —S—, —SO—, —SO₂—, or —NR⁷—, the number of carbon atoms before replacement corresponds to the defined number of carbon atoms in the hydrocarbon group.

Examples of the group formed by replacing —CH₂— contained in the hydrocarbon group with —O—, —CO—, —S—, —SO—, —SO₂—, or —NR⁷— include: a hydroxy group (a group formed by replacing —CH₂— contained in a methyl group with —O—), a carboxy group (a group formed by replacing —CH₂—CH₂— contained in an ethyl group with —O—CO—), a thiol group (a group formed by replacing —CH₂— contained in a methyl group with —S—), an amino group (a group formed by replacing —CH₂— contained in a methyl group with —NR⁷—), an oxy group (a group formed by replacing —CH₂— contained in a methylene group with —O—), a carbonyl group (a group formed by replacing —CH₂— contained in a methylene group with —CO—), a thio group (a group formed by replacing —CH₂— contained in a methylene group with —S—), a sulfonyl group (a group formed by replacing —CH₂— contained in a methylene group with —SO₂—), a peptide group (a group formed by replacing —CH₂—CH₂— contained in an ethylene group with —CO—NR⁷—), an alkoxy group (a group formed by replacing —CH₂— at any position contained in an alkyl group with —O—), an alkylthio group (a group formed by replacing —CH₂— at any position contained in an alkyl group with —S—), an alkylsulfonyl group (a group formed by replacing —CH₂— at any position contained in an alkyl group with —SO₂—), an alkylamino group (a group formed by replacing —CH₂— at any position contained in an alkyl group with —NR⁷—), an alkyl peptide group (a group formed by replacing —CH₂—CH₂— at any position contained in an alkyl group with —CO—NR⁷—), an alkoxycarbonyl group (a group formed by replacing —CH₂—CH₂— at any position contained in an alkyl group with —O—CO—), an alkylcarbonyl group (a group formed by replacing —CH₂— at any position contained in an alkyl group with —CO—), an alkylcarbonyloxy group (a group formed by replacing —CH₂—CH₂— at any position contained in an alkyl group with —CO—O—), an alkoxycarbonyloxy group (a group formed by replacing —CH₂—CH₂—CH₂— at any position contained in an alkyl group with —O—CO—O—), an alkoxyalkoxy group (a group formed by replacing two —CH₂— at any positions contained in an alkyl group with —O—), an alkoxyalkyl group (a group formed by replacing —CH₂— at any position contained in an alkyl group with —O—), a hydroxyalkyl group (a group formed by replacing —CH₂— contained in a methyl group at any position contained in an alkyl group with —O—), an alkanediyloxy group (a group formed by replacing —CH₂— at any position contained in an alkanediyl group with —O—), an alkanediyloxycarbonyl group (a group formed by replacing —CH₂—CH₂— at any position contained in an alkanediyl group with —O—CO—), an alkanediylcarbonyl group (a group formed by replacing —CH₂— at any position contained in an alkanediyl group with —CO—), an alkanediyl carbonyloxy group (a group formed by replacing —CH₂—CH₂— at any position contained in an alkanediyl group with —CO—O—), an alkanediyloxycarbonyloxy group (a group formed by replacing —CH₂—CH₂—CH₂— at any position contained in an alkanediyl group with —O—CO—O—), an alkanediylsulfonyl group (a group formed by replacing —CH₂— at any position contained in an alkanediyl group with —SO₂—), an alkanediylthio group (a group formed by replacing —CH₂— at any position contained in an alkanediyl group with —S—), an alkanediylamino group (a group formed by replacing —CH₂— at any position contained in an alkanediyl group with —NR⁷—), an alkanediyl peptide group (a group formed by replacing —CH₂—CH₂— at any position contained in an alkanediyl group with —CO—NR⁷—), a cycloalkoxy group, a cycloalkylalkoxy group, an aromatic hydrocarbon group-carbonyloxy group, an aromatic hydrocarbon group-carbonyl group, an aromatic hydrocarbon group-oxy group, and a combined group of two or more of these groups.

Examples of the alkoxy group include alkoxy groups having 1 to 27 carbon atoms, and examples thereof include a methoxy group, an ethoxy group, a propoxy group, a butoxy group, a pentyloxy group, a hexyloxy group, an octyloxy group, a 2-ethylhexyloxy group, a nonyloxy group, a decyloxy group and an undecyloxy group. The number of carbon atoms in an alkoxy group is preferably 1 to 11, more preferably 1 to 6, further preferably 1 to 4, and still more preferably 1 to 3.

Examples of the alkylthio group include alkylthio groups having 1 to 27 carbon atoms, and examples thereof include a methylthio group, an ethylthio group, a propylthio group, a butylthio group, a pentylthio group, a hexylthio group, an octylthio group, a 2-ethylhexylthio group, a nonylthio group, a decylthio group and an undecylthio group. The number of carbon atoms in the alkylthio group is preferably 1 to 11, more preferably 1 to 6, further preferably 1 to 4, and still more preferably 1 to 3.

Examples of the alkylsulfonyl group include alkylsulfonyl groups having 1 to 27 carbon atoms, and examples thereof include a methylsulfonyl group, an ethylsulfonyl group, a propylsulfonyl group, a butylsulfonyl group, a pentylsulfonyl group, a hexylsulfonyl group, an octylsulfonyl group, a 2-ethylhexylsulfonyl group, a nonylsulfonyl group, a decylsulfonyl group and an undecylsulfonyl group. The number of carbon atoms in the alkylsulfonyl group is preferably 1 to 11, more preferably 1 to 6, further preferably 1 to 4, and still more preferably 1 to 3.

Examples of the alkylamino group include alkylamino groups having 1 to 27 carbon atoms, and examples thereof include a methylamino group, an ethylamino group, a dimethylamino group, a propylamino group, a butylamino group, a pentylamino group, a pentylamino group and a hexylamino group. The number of carbon atoms of the alkylamino group is preferably 1 to 11, is more preferably 1 to 6, is further preferably 1 to 4, and is further more preferably 1 to 3.

Examples of the alkyl peptide group include alkyl peptide groups having 1 to 27 carbon atoms, and examples thereof include a methyl peptide group, an ethyl peptide group and a propyl peptide group. The number of carbon atoms of the alkyl peptide group is preferably 1 to 11, is more preferably 1 to 6, is further preferably 1 to 4, and is further more preferably 1 to 3.

Examples of the alkoxycarbonyl group include alkoxycarbonyl groups having 2 to 27 carbon atoms, and examples thereof include a methoxycarbonyl group, an ethoxycarbonyl group and a butoxycarbonyl group. Examples of the alkylcarbonyl group include alkylcarbonyl groups having 2 to 28 carbon atoms, and examples thereof include an acetyl group, a propionyl group and a butyryl group. Examples of the alkylcarbonyloxy group include alkylcarbonyloxy groups having 2 to 27 carbon atoms, and examples thereof include an acetyloxy group, a propionyloxy group and a butyryloxy group. Examples of the alkoxycarbonyloxy group include alkoxycarbonyloxy groups having 2 to 26 carbon atoms, and examples thereof include a butoxycarbonyloxy group. Examples of the alkoxyalkoxy group include alkoxyalkoxy groups having 2 to 26 carbon atoms, and examples thereof include a methoxymethoxy group, a methoxyethoxy group, an ethoxymethoxy group and an ethoxyethoxy group. The number of carbon atoms in an alkoxycarbonyl group is preferably 2 to 11, more preferably 2 to 6, further preferably 2 to 4, and still more preferably 2 or 3. The number of carbon atoms in an alkylcarbonyl group is preferably 2 to 12, more preferably 2 to 6, further preferably 2 to 4, and still more preferably 2 or 3. The number of carbon atoms in an alkylcarbonyloxy group is preferably 2 to 11, more preferably 2 to 6, further preferably 2 to 4, and still more preferably 2 or 3. The number of carbon atoms in an alkoxycarbonyloxy group is preferably 2 to 10, more preferably 2 to 6, further preferably 2 to 4, and still more preferably 2 or 3. The number of carbon atoms of the alkoxyalkoxy group is preferably 2 to 10, is more preferably 2 to 6, is further preferably 2 to 4, and is further more preferably 2 or 3.

Examples of the alkoxyalkyl group include alkoxyalkyl groups having 2 to 27 carbon atoms, and examples thereof include a methoxymethyl group, a methoxyethyl group, an ethoxymethyl group, an ethoxyethyl group, an ethoxypropyl group, an ethoxybutyl group, and a propoxymethyl group. The number of carbon atoms of the alkoxyalkyl group is preferably 2 to 11, is more preferably 2 to 6, is further preferably 2 to 4, and is further more preferably 2 or 3.

Examples of the hydroxyalkyl group include hydroxyalkyl groups having 1 to 27 carbon atoms, and examples thereof include a hydroxymethyl group, a dihydroxymethyl group, a hydroxyethyl group, a dihydroxyethyl group and a hydroxypropyl group. The number of carbon atoms of the hydroxyalkyl group is preferably 1 to 11, is more preferably 1 to 6, is further preferably 1 to 4, and is further more preferably 1 to 3.

Examples of the alkanediyloxy group include alkanediyloxy groups having 1 to 27 carbon atoms, and examples thereof include a methyleneoxy group, an ethyleneoxy group, a propanediyloxy group, a butanediyloxy group and a pentanediyloxy group. The number of carbon atoms of the alkanediyloxy group is preferably 1 to 11, is more preferably 1 to 6, is further preferably 1 to 4, and is further more preferably 1 to 3.

Examples of the alkanediyloxycarbonyl group include alkanediyloxycarbonyl groups having 2 to 27 carbon atoms, and examples thereof include a methyleneoxycarbonyl group, an ethyleneoxycarbonyl group, a propanediyloxycarbonyl group and a butanediyloxycarbonyl group. Examples of the alkanediyl carbonyl group include alkanediylcarbonyl groups having 2 to 28 carbon atoms, and examples thereof include a methylenecarbonyl group, an ethylenecarbonyl group, a propanediylcarbonyl group, a butanediylcarbonyl group and a pentanediylcarbonyl group. Examples of the alkanediylcarbonyloxy group include alkanediyl carbonyloxy groups having 2 to 27 carbon atoms, and examples thereof include a methylenecarbonyloxy group, an ethylenecarbonyloxy group, a propanediylcarbonyloxy group and a butanediylcarbonyloxy group. Examples of the alkanediyloxycarbonyloxy group include alkoxycarbonyloxy groups having 2 to 26 carbon atoms, and examples thereof include a butoxycarbonyloxy group. The number of carbon atoms of the alkanediyloxycarbonyl group is preferably 2 to 11, is more preferably 2 to 6, is further preferably 2 to 4, and is further more preferably 2 or 3. The number of carbon atoms of the alkanediyl carbonyl group is preferably 2 to 12, more preferably 2 to 6, further preferably 2 to 4, and still more preferably 2 or 3. The number of carbon atoms of the alkanediylcarbonyloxy group is preferably 2 to 11, is more preferably 2 to 6, is further preferably 2 to 4, and is further more preferably 2 or 3. The number of carbon atoms of the alkanediyloxycarbonyloxy group is preferably 2 to 10, is more preferably 2 to 6, is further preferably 2 to 4, and is further more preferably 2 or 3.

Examples of the alkanediylsulfonyl group include alkanediylsulfonyl groups having 1 to 27 carbon atoms, and examples thereof include a methylenesulfonyl group, an ethylenesulfonyl group and a propylenesulfonyl group. The number of carbon atoms of the alkanediylsulfonyl group is preferably 1 to 11, is more preferably 1 to 6, is further preferably 1 to 4, and is further more preferably 1 to 3.

Examples of the alkanediylthio group include alkanediylthio groups having 1 to 27 carbon atoms, and examples thereof include a methylenethio group, an ethylenethio group and a propylenethio group. The number of carbon atoms of the alkanediylthio group is preferably 1 to 11, is more preferably 1 to 6, is further preferably 1 to 4, and is further more preferably 1 to 3.

Examples of the alkanediylamino group include alkanediylamino groups having 1 to 27 carbon atoms, and examples thereof include a methyleneamino group, an ethyleneamino group, a dimethyleneamino group, a propyleneamino group, a butyleneamino group, a pentyleneamino group and a hexyleneamino group. The number of carbon atoms of the alkanediylamino group is preferably 1 to 11, is more preferably 1 to 6, is further preferably 1 to 4, and is further more preferably 1 to 3.

Examples of the alkanediyl peptide group include alkanediyl peptide groups having 1 to 27 carbon atoms, and examples thereof include a methylene peptide group, an ethylene peptide group and a propylene peptide group. The number of carbon atoms of the alkanediyl peptide group is preferably 1 to 11, is more preferably 1 to 6, is further preferably 1 to 4, and is further more preferably 1 to 3.

Examples of the cycloalkoxy group include cycloalkoxy groups having 3 to 27 carbon atoms, and examples thereof include a cyclohexyloxy group. Examples of the cycloalkylalkoxy group include cycloalkylalkoxy groups having 4 to 27 carbon atoms, and examples thereof include a cyclohexylmethoxy group. Examples of the aromatic hydrocarbon group-carbonyloxy group include aromatic hydrocarbon group-carbonyloxy groups having 7 to 27 carbon atoms, and examples thereof include a benzoyloxy group. Examples of the aromatic hydrocarbon group-carbonyl group include aromatic hydrocarbon group-carbonyl groups having 7 to 28 carbon atoms, and examples thereof include a benzoyl group. Examples of the aromatic hydrocarbon group-oxy group include aromatic hydrocarbon group-oxy groups having 6 to 27 carbon atoms, and examples thereof include a phenyloxy group.

In addition, examples of the group formed by replacing —CH₂— contained in the alicyclic hydrocarbon group, the aromatic hydrocarbon group, or the combined group of these groups with —O—, —CO—, —S—, —SO—, —SO₂—, or —NR⁷— include the following groups. In the groups shown below, —O— or —CO— is optionally replaced with —S—, —SO—, —SO₂—, or —NR⁷—, respectively. The bonding site can be set to any position.

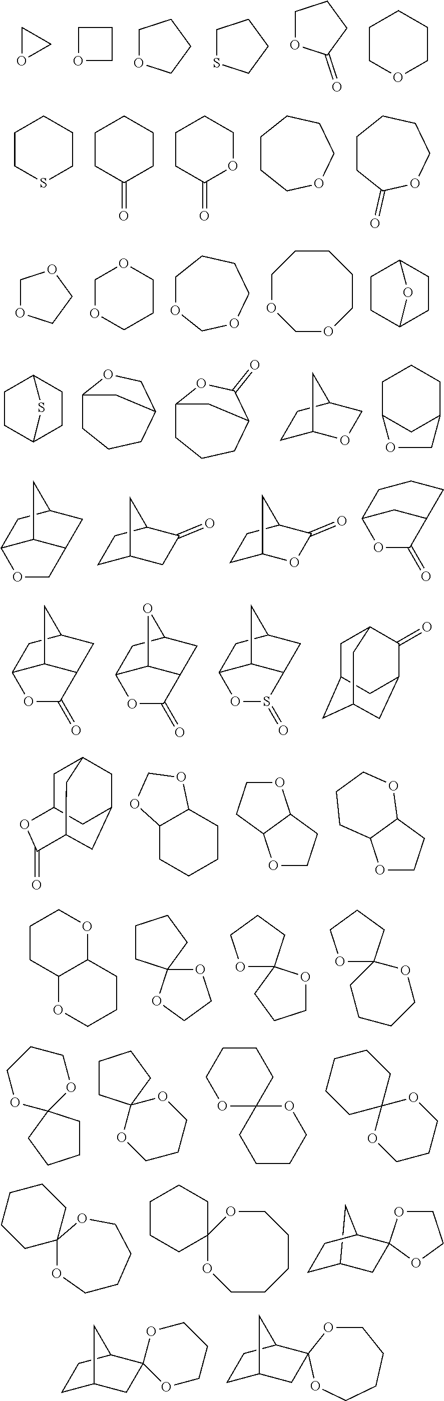

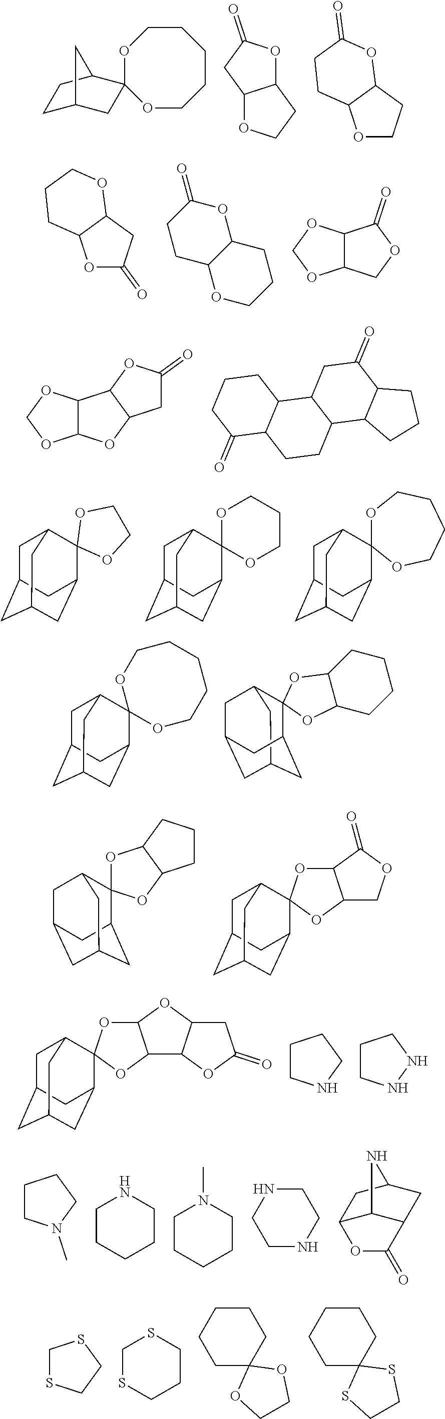

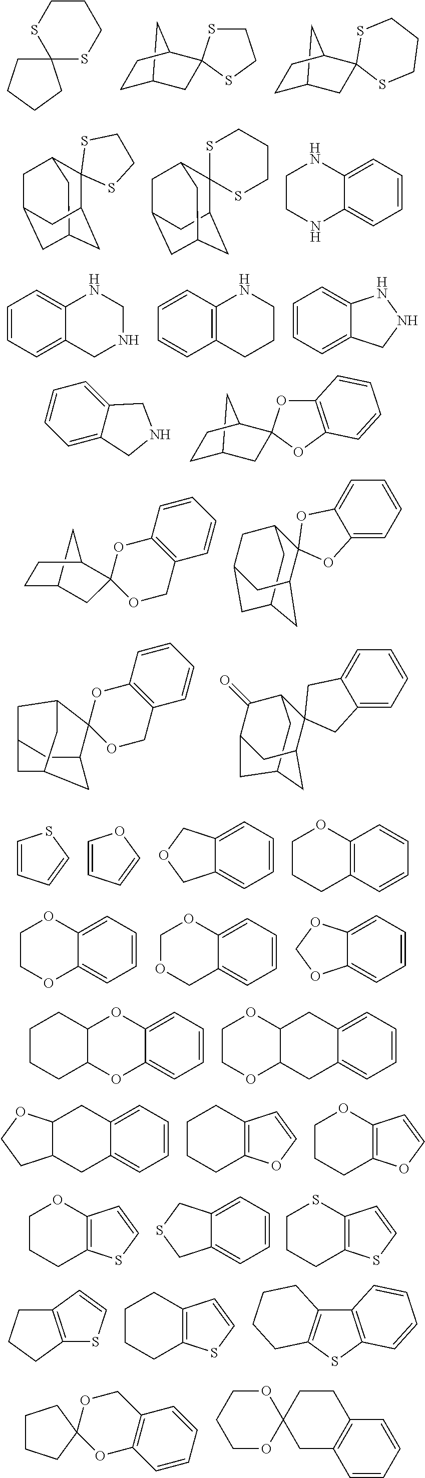

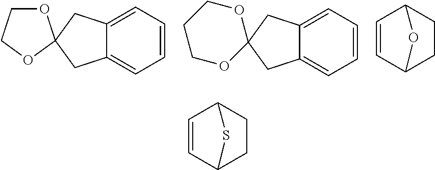

The substituents that L¹ and L² optionally have include a halogen atom, a cyano group and a nitro group. By replacing —CH₂— contained in the chain hydrocarbon group such as an alkyl group contained in L¹ and L² with —O—, —S—, —CO—, —SO—, —NR⁷—, or —SO₂—, L¹ and L² can substantially have substituents such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group, and an alkyl peptide group. In addition, when L¹ and L² are combined groups of an alicyclic hydrocarbon group or an aromatic hydrocarbon group and a chain hydrocarbon group such as an alkyl group, the chain hydrocarbon group such as the alkyl group can be substantially a substituent of an alicyclic hydrocarbon group or an aromatic hydrocarbon group.

Examples of the halogen atom include a fluorine atom, a chlorine atom, a bromine atom and an iodine atom.

Examples of L¹ include a single bond, a chain hydrocarbon group having 1 to 18 carbon atoms and optionally having a substituent (—CH₂— contained in the chain hydrocarbon group is optionally replaced with —O—, —CO—, —S—, —SO—, —SO₂—, or —NR⁷—), a cyclic hydrocarbon group having 3 to 18 carbon atoms and optionally having a substituent (—CH₂— contained in the cyclic hydrocarbon group is optionally replaced with —O—, —CO—, —S—, —SO—, —SO₂—, or —NR⁷—), or a combined group of these groups. A single bond, a chain hydrocarbon group having 1 to 10 carbon atoms and optionally having a substituent (—CH₂— contained in the chain hydrocarbon group is optionally replaced with —O—, —CO—, —S—, —SO—, —SO₂—, or —NR⁷—), a cyclic hydrocarbon group having 3 to 18 carbon atoms and optionally having a substituent (—CH₂— contained in the cyclic hydrocarbon group is optionally replaced with —O—, —CO—, —S—, —SO—, —SO₂—, or —NR⁷—), or a combined group of these groups are preferable, a single bond, a chain hydrocarbon group having 1 to 8 carbon atoms (—CH₂— contained in the chain hydrocarbon group is optionally replaced with —O— or —CO—), a combined group of a chain hydrocarbon group having 1 to 8 carbon atoms and an alicyclic hydrocarbon group having 3 to 18 carbon atoms (—CH₂— contained in the chain hydrocarbon group is optionally replaced with —O— or —CO—, and —CH₂— contained in the alicyclic hydrocarbon group is optionally replaced with —O—, —CO—, —S—, —SO—, —SO₂, or —NR⁷—), or a combined group of a chain hydrocarbon group having 1 to 8 carbon atoms and an aromatic hydrocarbon group having 6 to 18 carbon atoms and optionally having a substituent (—CH₂— contained in the chain hydrocarbon group is optionally replaced with —O— or —CO—) are more preferable, a single bond, a chain hydrocarbon group having 1 to 6 carbon atoms (—CH₂— contained in the chain hydrocarbon group is optionally replaced with —O— or —CO—), a combined group of a chain hydrocarbon group having 1 to 4 carbon atoms and an alicyclic hydrocarbon group having 3 to 12 carbon atoms (—CH₂— contained in the chain hydrocarbon group is optionally replaced with —O— or —CO—, and —CH₂— contained in the alicyclic hydrocarbon group is optionally replaced with —O— or —CO—), or a combined group of a chain hydrocarbon group having 1 to 4 carbon atoms and an aromatic hydrocarbon group having 6 to 10 carbon atoms and optionally having a substituent (—CH₂— contained in the chain hydrocarbon group is optionally replaced with —O— or —CO—) are still more preferable.

Examples of L² include a single bond, a chain hydrocarbon group having 1 to 18 carbon atoms and optionally having a substituent (—CH₂— contained in the chain hydrocarbon group is optionally replaced with —O—, —CO—, —S—, —SO—, —SO₂—, or —NR⁷—), a cyclic hydrocarbon group having 3 to 18 carbon atoms and optionally having a substituent (—CH₂— contained in the cyclic hydrocarbon group is optionally replaced with —O—, —CO—, —S—, —SO—, —SO₂—, or —NR⁷—), or a combined group of these groups. A single bond, a chain hydrocarbon group having 1 to 10 carbon atoms and optionally having a substituent (—CH₂— contained in the chain hydrocarbon group is optionally replaced with —O—, —CO—, —S—, —SO—, —SO₂—, or —NR⁷—), a cyclic hydrocarbon group having 3 to 18 carbon atoms and optionally having a substituent (—CH₂— contained in the cyclic hydrocarbon group is optionally replaced with —O—, —CO—, —S—, —SO—, —SO₂—, or —NR⁷—), or a combined group of these groups are preferable, a single bond or a chain hydrocarbon group having 1 to 10 carbon atoms (—CH₂— contained in the chain hydrocarbon group is optionally replaced with —O— or —CO—) are more preferable, a single bond or a straight-chain hydrocarbon group having 1 to 6 carbon atoms (—CH₂— contained in the chain hydrocarbon group is optionally replaced with —O— or —CO—) are further preferable, a single bond or a methylene group are further more preferable, and a methylene group is even still more preferable.

Examples of the alicyclic hydrocarbon group of W¹ include a monocyclic or polycyclic (including a spiro ring, a condensed ring or bridged ring) alicyclic hydrocarbon group. The carbon atoms contained in the alicyclic hydrocarbon group may form a double bond between two adjacent carbon atoms. The alicyclic hydrocarbon group of W¹ is a (mm2+1)-valent alicyclic hydrocarbon group, which may be a divalent to hexavalent alicyclic hydrocarbon group depending on mm2. The number of carbon atoms in the alicyclic hydrocarbon group is preferably 3 to 30, more preferably 3 to 24, and further preferably 3 to 20.

Examples of monocyclic alicyclic hydrocarbon groups include monocyclic cycloalkanediyl groups, and examples thereof include cyclopropanediyl groups, cyclobutanediyl groups, cyclopentanediyl groups, cyclohexanediyl groups, cycloheptanediyl groups, cyclooctanediyl groups, and cyclodecanediyl groups.

Examples of the polycyclic alicyclic hydrocarbon groups include polycyclic cycloalkanediyl groups, and examples thereof include cycloalkanediyl groups having cross-linked structures, cycloalkanediyl groups in which two or more rings are condensed, or cycloalkanediyl groups in which two rings are bonded by spiro. Examples of the cycloalkanediyl groups having cross-linked structures include a norbornanediyl group and an adamantanediyl group. Examples of the cycloalkanediyl groups in which two or more rings are condensed include a bicyclo[4,4,0]decane group and a steroid group (steroid skeleton). Examples of the cycloalkanediyl groups in which two rings are bonded by spiro include spiro-ring cycloalkanediyl groups in which one cycloalkanediyl group selected from the group consisting of cyclopentanediyl groups, cyclohexanediyl groups, norbornanediyl groups, and adamantanediyl groups and a cycloalkanediyl group having 5 to 8 carbon atoms are bonded by spiro. A double bond may be formed between two carbon atoms contained in the alicyclic hydrocarbon group.

More specifically, examples thereof include alicyclic hydrocarbon groups represented by the following formulae.

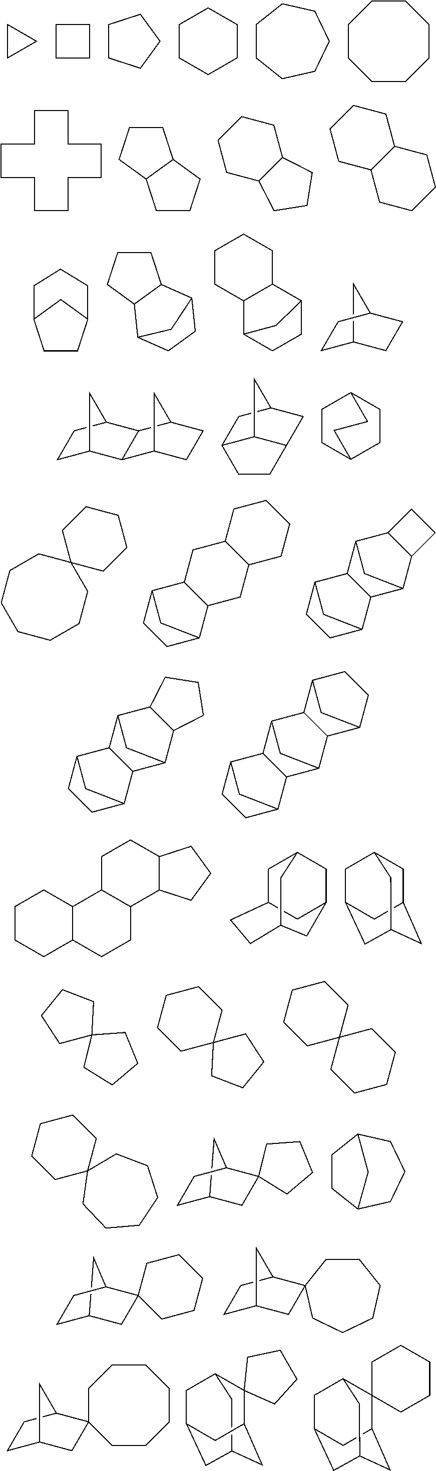

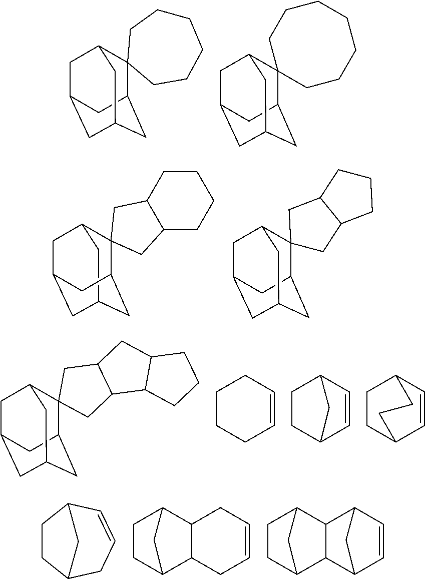

In a case where the alicyclic hydrocarbon group is a monocyclic alicyclic hydrocarbon group, the number of carbon atoms of the alicyclic hydrocarbon group is preferably 3 to 24, is more preferably 3 to 20, is further preferably 3 to 18, is far more preferably 3 to 12, is still further preferably 3 to 10, and is even more preferably 3 to 8. In a case where the alicyclic hydrocarbon group is a polycyclic alicyclic hydrocarbon group, the number of carbon atoms of the alicyclic hydrocarbon group is preferably 6 to 24, is more preferably 6 to 20, is further preferably 6 to 18, is far more preferably 6 to 12, and is still further preferably 7 to 12.

—CH₂— contained in the alicyclic hydrocarbon group is optionally replaced with —O—, —CO—, —S—, —SO—, —SO₂—, or —NR⁷—, and examples of such substituted groups include the following groups and groups obtained by replacing —O— with —S—, and groups obtained by replacing —CO— with —SO—, —SO₂—, or —NR⁷— as described below. The bonding site can be set to any position.

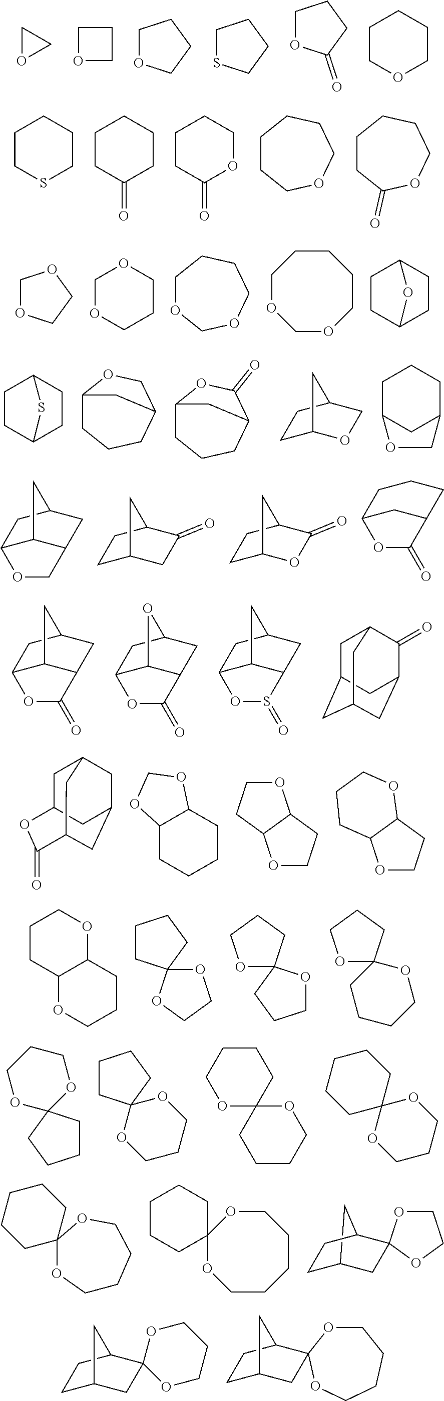

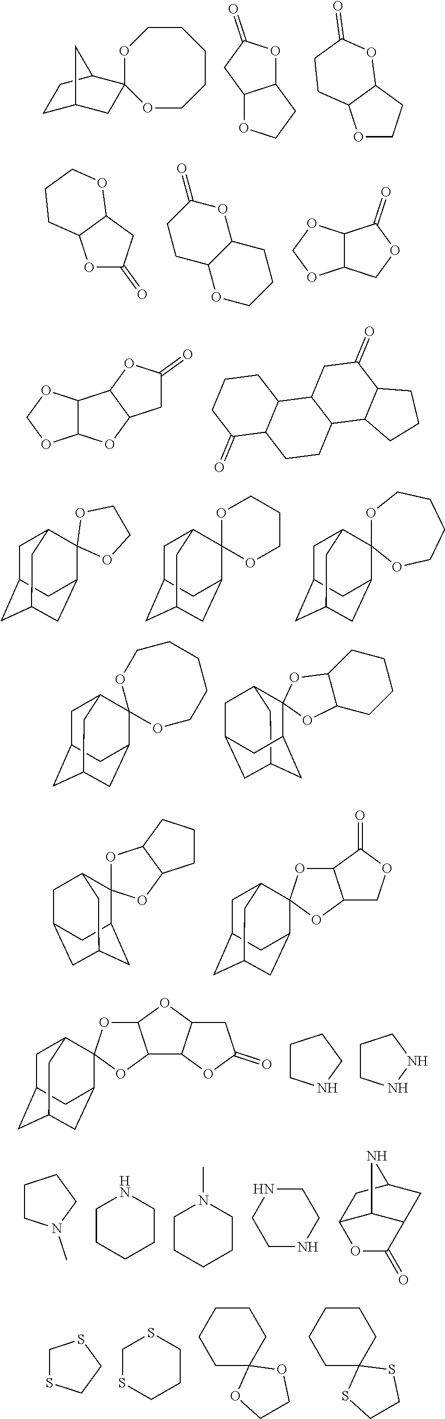

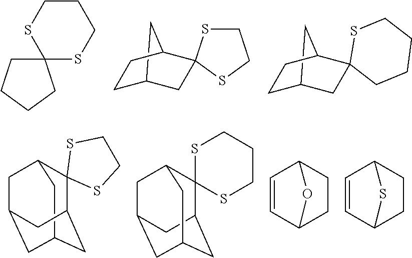

The alicyclic hydrocarbon group is preferably an alicyclic hydrocarbon group having 3 to 18 carbon atoms (the alicyclic hydrocarbon group optionally has a substituent, and —CH₂— contained in the alicyclic hydrocarbon group is optionally replaced with —O—, —S—, —SO₂—, —SO—, —NR⁷—, or —CO—), and is more preferably a group represented by any of formula (w1-1) to formula (w1-29).

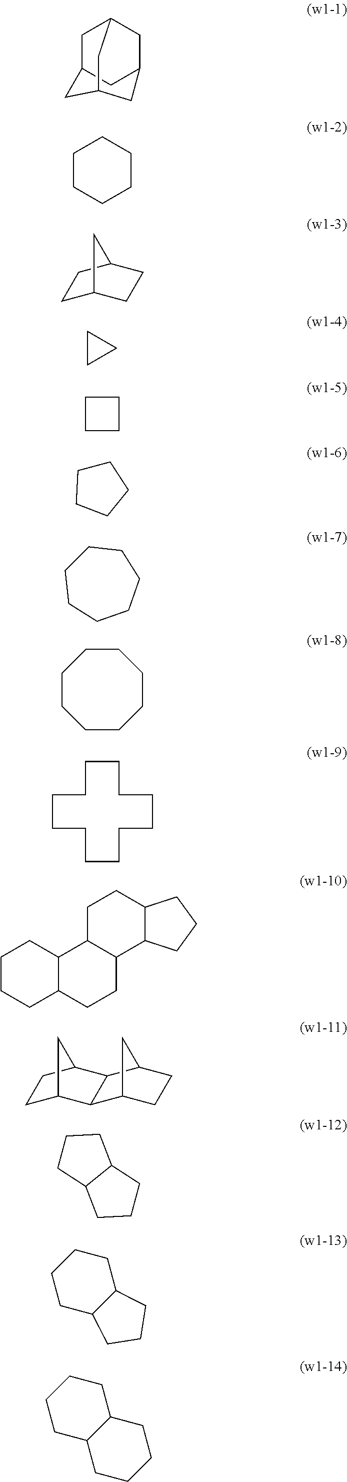

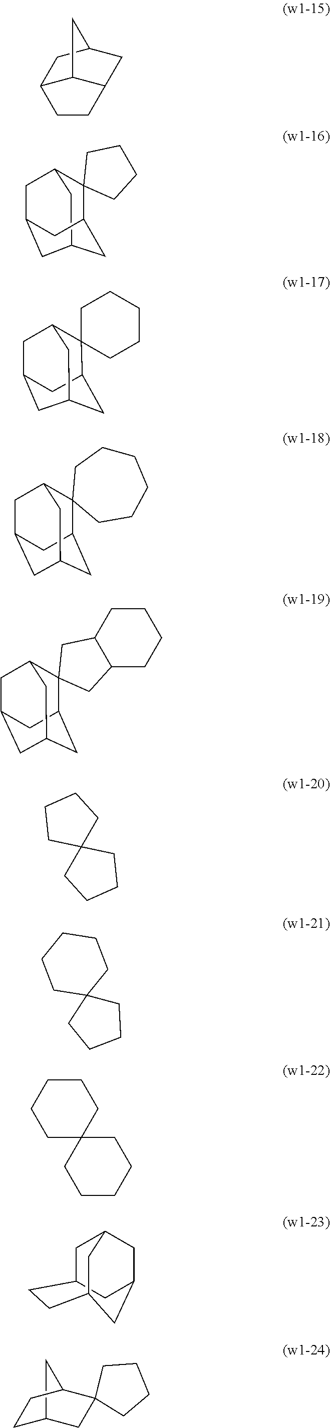

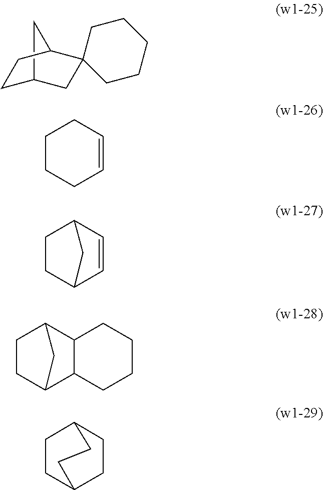

[In the formula (w1-1) to formula (w1-29),
the group optionally has a substituent, and —CH₂— contained in the group is optionally replaced with —O—, —S—, —CO—, —SO—, —NR⁷—, or —SO₂. The bonding site may be at any position.]

Examples of the substituent that W¹ may have include a fluorine atom, a chlorine atom, a bromine atom, a cyano group, a nitro group, a haloalkyl group having 1 to 6 carbon atoms, an alkyl group having 1 to 12 carbon atoms (where —CH₂— contained in the alkyl group may be replaced with —O—, —S—, —SO₂—, —SO—, —NR⁷—, or —CO—).

Examples of the haloalkyl group having 1 to 6 carbon atoms include a fluorinated alkyl group having 1 to 6 carbon atoms, a chlorinated alkyl group having 1 to 6 carbon atoms, a brominated alkyl group having 1 to 6 carbon atoms, and an iodinated alkyl group having 1 to 6 carbon atoms. Examples of the haloalkyl group include a perfluoroalkyl group having 1 to 6 carbon atoms (such as a trifluoromethyl group, a pentafluoroethyl group, a heptafluoropropyl group, and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, an iodomethyl group, a fluoromethyl group, and a difluoromethyl group. The number of carbon atoms of the haloalkyl group is preferably 1 to 4, and is more preferably 1 to 3.

Examples of the alkyl group having 1 to 12 carbon atoms include alkyl groups such as a methyl group, an ethyl group, a propyl group, an isopropyl group, a butyl group, an isobutyl group, a tert-butyl group, a pentyl group, a hexyl group, an octyl group, and a nonyl group. The number of carbon atoms of the alkyl group is preferably 1 to 9, is more preferably 1 to 6, and is further preferably 1 to 4.

When —CH₂— in the alkyl group is replaced with —O—, —CO—, —S—, —SO—, —NR⁷—, or —SO₂—, the number of carbon atoms before the replacement is regarded as the total number of carbon atoms in the alkyl group.

Examples of substituted groups include hydroxy groups, carboxy groups, oxy groups, carbonyl groups, alkoxy groups, alkoxycarbonyl groups, alkylcarbonyl groups, alkylcarbonyloxy groups, thiol groups, alkylthio groups, alkylsulfonyl groups, alkoxycarbonyloxy groups, alkoxyalkoxy groups, amino groups, alkylamino groups, alkylpeptide groups, and groups formed by combining two or more of these groups. Examples of these substituted groups include the same groups as the groups exemplified in this specification, within the range permitted by the number of carbon atoms.

The number of substituents that W¹ may have is not particularly limited, but it is preferably 1, 2, or 4.

Among them, W¹ may be an alicyclic hydrocarbon group having 3 to 24 carbon atoms (—CH₂— contained in the alicyclic hydrocarbon group is optionally replaced with —O—, —S—, —SO₂—, —SO—, —NR⁷—, or —CO—, and the alicyclic hydrocarbon group may have a substituent), preferably an alicyclic hydrocarbon group having 3 to 18 carbon atoms (—CH₂— contained in the alicyclic hydrocarbon group is optionally replaced with —O—, —S—, —SO₂—, —SO—, —NR⁷—, or —CO—, and the alicyclic hydrocarbon group may have a substituent), more preferably an alicyclic hydrocarbon group having 5 to 18 carbon atoms and optionally having a substituent, still more preferably an adamantanediyl group, a cyclohexanediyl group, or a cyclopentanediyl group, and even still more preferably a cyclohexanediyl group or an adamantanediyl group.

Examples of the hydrocarbon group of A¹ include a straight-chain or branched chain hydrocarbon group (for example, an alkanediyl group), a monocyclic or polycyclic (including a spiro ring, a condensed ring or bridged ring) alicyclic hydrocarbon group, a saturated or unsaturated alicyclic hydrocarbon group, and an aromatic hydrocarbon group. It may be the combined group of two or more of these groups (for example, a hydrocarbon group formed from an alicyclic hydrocarbon group or an aromatic hydrocarbon group and a chain hydrocarbon group). The number of carbon atoms of the chain hydrocarbon group is preferably 1 to 19, is more preferably 1 to 18, is further preferably 1 to 16, is far more preferably 1 to 14, and is still further preferably 1 to 12.

Examples of the chain hydrocarbon group include: straight-chain alkanediyl groups such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group and a heptadecane-1,17-diyl group; and branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group. The terminal of the branched alkanediyl group may be an alkyl group such as a methyl group.

The number of carbon atoms in the chain hydrocarbon group is preferably 1 to 18, more preferably 1 to 12, further preferably 1 to 10, still more preferably 1 to 9, even more preferably 1 to 8, further more preferably 1 to 6, even further more preferably 1 to 4, and even still more preferably 1 to 3.

The alicyclic hydrocarbon group may be a monocyclic or polycyclic hydrocarbon group, and examples thereof include the following groups. The bonding site can be set to any position.

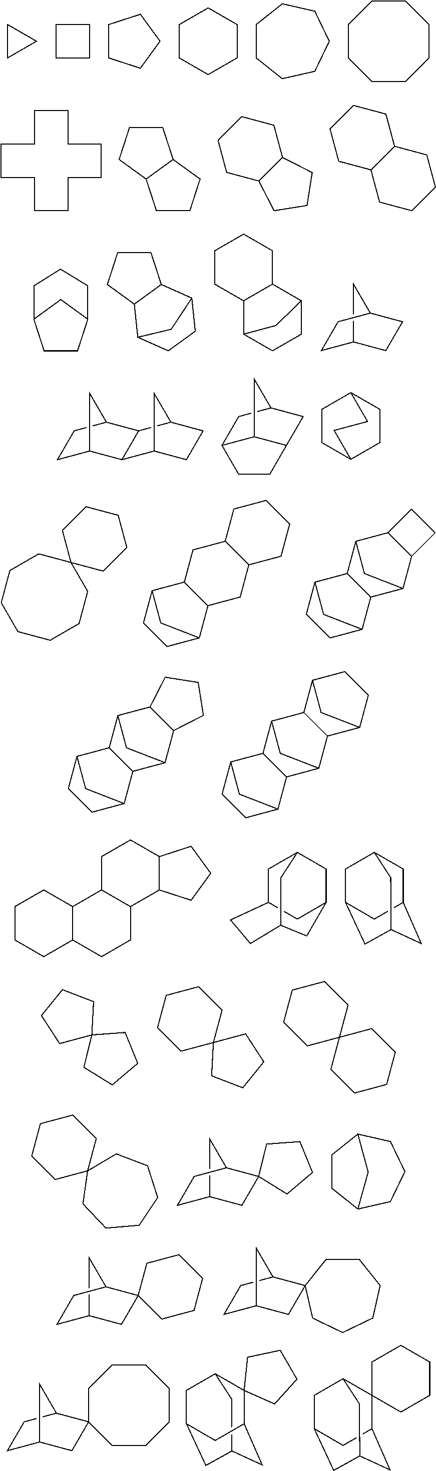

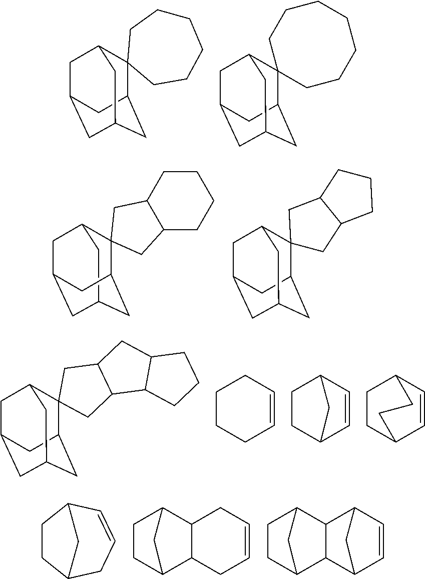

Specific examples thereof include: monocyclic alicyclic hydrocarbon groups that are monocyclic cycloalkanediyl groups such as a cyclobutane-1,3-diyl group, a cyclopentane-1,3-diyl group, a cyclohexane-1,4-diyl group and a cyclooctane-1,5-diyl group; and
polycyclic alicyclic hydrocarbon groups that are polycyclic cycloalkanediyl groups such as a norbornane-1,4-diyl group, a norbornane-2,5-diyl group, an adamantane-1,5-diyl group, an adamantane-2,6-diyl group, cycloalkyl groups such as spirocyclohexane-1,2′-cyclopentane group and spiroadamantane-2,3′-cyclopentane group, and a spiro ring having a cycloalkyl group bonded to each of a norbornyl group and an adamantyl group by spiro.

The number of carbon atoms in the alicyclic hydrocarbon group is preferably 3 to 18, more preferably 3 to 16, further preferably 3 to 12, and still more preferably 3 to 10.

Examples of the aromatic hydrocarbon groups include arylene groups such as a phenylene group, a naphthylene group, an anthrylene group, a biphenylene group and a phenanthrylene group. The number of carbon atoms of the aromatic hydrocarbon group is preferably 6 to 18, is more preferably 6 to 14, and is further preferably 6 to 10.

Examples of the group formed by combining two or more groups include a combined group of an alicyclic hydrocarbon group and a chain hydrocarbon group, a combined group of an aromatic hydrocarbon group and a chain hydrocarbon group, and a combined group of an alicyclic hydrocarbon group and an aromatic hydrocarbon group. In the combination, two or more types of chain hydrocarbon groups, alicyclic hydrocarbon groups, and aromatic hydrocarbon groups may each be combined. In addition, any of the groups may be bonded to Ar¹.

Examples of the combined group of an alicyclic hydrocarbon group and a chain hydrocarbon group include -(divalent alicyclic hydrocarbon group)-(alkanediyl group)-, -(alkanediyl group)-(divalent alicyclic hydrocarbon group)-(alkanediyl group)-, and -(alkanediyl group)-(divalent alicyclic hydrocarbon group)-.

Examples of the combined group of an aromatic hydrocarbon group and a chain hydrocarbon group include -(divalent aromatic hydrocarbon group)-(alkanediyl group)-, -(alkanediyl group)-(divalent aromatic hydrocarbon group)-(alkanediyl group)-, and -(alkanediyl group)-(divalent aromatic hydrocarbon group)-.

Examples of the combined group of an alicyclic hydrocarbon group and an aromatic hydrocarbon group include -aromatic hydrocarbon group-alicyclic hydrocarbon group-, -alicyclic hydrocarbon group-aromatic hydrocarbon group-, and -alicyclic hydrocarbon group-aromatic hydrocarbon group-alicyclic hydrocarbon group-.

When —CH₂— contained in the hydrocarbon group for A¹ is replaced with —O—, —S—, —SO₂—, —SO—, —NR⁷—, or —CO—, the number of carbon atoms before replacement corresponds to the defined number of carbon atoms in the hydrocarbon group. Moreover, its number may be one or two or more, but is preferably one to three.

Examples of the group obtained by replacing —CH₂— contained in the hydrocarbon group with —O—, —S—, —SO₂—, —SO—, —NR⁷—, or —CO— include hydroxy groups, carboxy groups, thiol groups, amino groups, oxy groups, carbonyl groups, thio groups, sulfonyl groups, peptide groups, alkoxy groups, alkylthio groups, alkylsulfonyl groups, alkylamino groups, alkylpeptide groups, alkoxycarbonyl groups, alkylcarbonyl groups, alkylcarbonyloxy groups, alkoxycarbonyloxy groups, alkoxycarbonyloxy groups, alkoxyalkoxy groups, alkoxyalkyl groups, hydroxyalkyl groups, alkanediyloxy groups, alkanediyloxycarbonyl groups, alkanediylcarbonyl groups, alkanediylcarbonyloxy groups, alkanediyloxycarbonyloxy groups, alkanediylsulfonyl groups, alkanediylthio groups, alkanediylamino groups, alkanediyl peptide groups, cycloalkoxy groups, cycloalkylalkoxy groups, aromatic hydrocarbon group-carbonyloxy groups, aromatic hydrocarbon group-carbonyl groups, aromatic hydrocarbon group-oxy groups, alicyclic hydrocarbon groups, groups obtained by replacing —CH₂— contained in the aromatic hydrocarbon group or the combined group thereof with —O—, —S—, —SO₂—, —NR⁷—, —SO—, or —CO—, and groups obtained by combining two or more groups thereof. Examples of these substituted groups include the same groups as the groups exemplified in this specification, within the range permitted by the number of carbon atoms.

Examples of the substituent that the hydrocarbon groups for A¹ may have include a halogen atom, a cyano group, and a nitro group. By replacing —CH₂— contained in the chain hydrocarbon group such as an alkyl group of A¹ with —O—, —S—, —SO₂—, —SO—, —NR⁷—, or —CO—, A¹ can substantially have substituents such as a hydroxy group, a carboxy group, an alkoxy group, an alkoxycarbonyl group, an alkylcarbonyl group, an alkylcarbonyloxy group, an alkoxycarbonyloxy group, an alkoxyalkoxy group, a thiol group, an alkylthio group, an alkylsulfonyl group, an amino group, an alkylamino group, and an alkyl peptide group. In addition, when A¹ is a combined group of an alicyclic hydrocarbon group or an aromatic hydrocarbon group and a chain hydrocarbon group such as an alkyl group, the chain hydrocarbon group such as the alkyl group can be substantially a substituent of an alicyclic hydrocarbon group or an aromatic hydrocarbon group.

Examples of the halogen atom include a fluorine atom, a chlorine atom, a bromine atom and an iodine atom.

A¹ is preferably a chain hydrocarbon group having 1 to 10 carbon atoms, a cyclic hydrocarbon group having 3 to 18 carbon atoms, or a combined group thereof (note that, the chain hydrocarbon group and the cyclic hydrocarbon group optionally have a substituent, and —CH₂— contained in the chain hydrocarbon group and the cyclic hydrocarbon group is optionally replaced with —O—, —S—, —SO₂—, —SO—, —NR⁷—, or —CO—), and is more preferably a chain hydrocarbon group having 1 to 8 carbon atoms (note that, —CH₂— contained in the chain hydrocarbon group is optionally replaced with —O—, —S—, —SO₂—, —SO—, —NR⁷—, or —CO—), a combined group formed of a chain hydrocarbon group having 1 to 6 carbon atoms and an alicyclic hydrocarbon group having 3 to 18 carbon atoms (note that, —CH₂— contained in the chain hydrocarbon group and the alicyclic hydrocarbon group is optionally replaced with —O—, —S—, —SO—, —SO₂—, —NR⁷—, or —CO—), or a combined group formed of a chain hydrocarbon group having 1 to 6 carbon atoms and an aromatic hydrocarbon group having 6 to 18 carbon atoms and optionally having a substituent (note that, —CH₂— contained in the chain hydrocarbon group is optionally replaced with —O—, —S—, —SO₂—, —SO—, —NR⁷—, or —CO—).

A¹ is also preferably a hydrocarbon group having 1 to 20 carbon atoms (the hydrocarbon group optionally has a substituent, and —CH₂— contained in the hydrocarbon group is optionally replaced with —O—, —CO—, —S—, —SO—, —NR⁷—, or —SO₂—. Note that, at least one of —CH₂— contained in the hydrocarbon groups is replaced with —O—, —CO—, —S—, —SO—, —NR⁷—, or —SO₂—).

In this case, for example, A¹ is preferably *—X^{1A}-A^{1A}-(X^{1A} represents *—CO—O—, *—O—CO—, *—O—CO—O—, *—O—, *—CO—NR⁷—, *—NR⁷—CO—, *—NR⁷—CO—, *—O—CO—NR⁷— or *—NR⁷—. * represents a bonding site to W¹. A^{1A} represents a single bond, a chain hydrocarbon group having 1 to 4 carbon atoms, a cyclic hydrocarbon group having 3 to 12 carbon atoms, or a combined group thereof, and —CH₂— contained in the chain hydrocarbon group and the cyclic hydrocarbon group is optionally replaced with —O—, —S—, —SO₂—, —SO—, —NR⁷—, or —CO—).

In addition, A¹ is more preferably ***-L⁰¹¹-X⁰¹-L⁰¹²-(X⁰¹ represents —O—, —CO—, —S—, —SO—, —NR⁷—, or —SO₂—. L⁰¹¹ and L⁰¹² each independently represent a single bond or a hydrocarbon group having 1 to 18 carbon atoms, the hydrocarbon group optionally has a substituent, and —CH₂— contained in the hydrocarbon group is optionally replaced with —O—, —CO—, —S—, —SO—, —NR⁷—, or —SO₂—. Note that, the total number of carbon atoms of L⁰¹¹ and L⁰¹² is 0 to 18. *** represents a bonding site to W¹.), and still more preferably ***—X⁰¹-L⁰¹- or ***-L⁰¹-X⁰¹— (X⁰¹ represents —O—, —CO—, —S—, —SO—, —NR⁷—, or —SO₂—. L⁰¹ represents a single bond or a hydrocarbon group having 1 to 18 carbon atoms, the hydrocarbon group optionally has a substituent, and —CH₂— contained in the hydrocarbon group is optionally replaced with —O—, —CO—, —S—, —SO—, —NR⁷—, or —SO₂—. *** represents a bonding site to W¹.).

Examples of the hydrocarbon group having 1 to 18 carbon atoms (the hydrocarbon group optionally has a substituent, and —CH₂— contained in the hydrocarbon group is optionally replaced with —O—, —CO—, —S—, —SO—, —NR⁷—, or —SO₂—.) of L⁰¹¹, L⁰¹², and L⁰¹ include the same groups as the groups exemplified in A¹ within the range that the number of carbon atoms is 1 to 18.

X⁰¹ is preferably —O—, —S—, —NR⁷—, or —CO—, and more preferably —O—, —NR⁷—, or —CO—.

L⁰¹¹, L⁰¹², and L⁰¹ are each independently preferably a single bond or a hydrocarbon group having 1 to 12 carbon atoms (—CH₂— contained in the hydrocarbon group is optionally replaced with —O—, —CO—, —S—, —SO—, —NR⁷—, or —SO₂—), more preferably a single bond or a chain hydrocarbon group having 1 to 9 carbon atoms (—CH₂— contained in the chain hydrocarbon group is optionally replaced with —O—, —CO—, —S—, —SO—, —NR⁷—, or —SO₂—), still more preferably a single bond or an alkanediyl group having 1 to 6 carbon atoms (—CH₂— contained in the alkanediyl group is optionally replaced with —O— or —CO—), further more preferably a single bond or an alkanediyl group having 1 to 5 carbon atoms (CH₂— contained in the alkanediyl group is optionally replaced with —O— or —CO—), still further more preferably a single bond or an alkanediyl group having 1 to 4 carbon atoms (CH₂— contained in the alkanediyl group is optionally replaced with —O— or —CO—), and even still further more preferably a single bond or an alkanediyl group having 1 to 3 carbon atoms (CH₂— contained in the alkanediyl group is optionally replaced with —O— or —CO—). Among them, a single bond or *1-L^{01A}-L^{01B}- (L^{01A} represents a single bond, —O—, or —CO—. L^{01B} represents a methylene group, an ethane-1,1-diyl group, a propane-1,1-diyl group, a propane-2,2-diyl group, an oxy group, a carbonyl group, a carbonyloxy group, a carbonyloxymethylene group, an ethyleneoxy group, a methylenecarbonyloxymethylene group, or an ethyleneoxycarbonyl group. *1 represents a bonding site to W¹ or X⁰¹.) are preferable, and a single bond, a methylene group, an oxy group, or a carbonyl group are more preferable.

From another perspective, A¹ is preferably a hydrocarbon group having 1 to 18 carbon atoms (the hydrocarbon group optionally has a substituent, and —CH₂— contained in the hydrocarbon group is optionally replaced with —O—, —CO—, —S—, —SO—, —NR⁷—, or —SO₂—.), more preferably a chain-like hydrocarbon group having 1 to 16 carbon atoms (the chain-like hydrocarbon group may have a substituent, and —CH₂— contained in the hydrocarbon group is optionally replaced with —O—, —CO—, —S—, or —SO₂—.), and still more preferably a chain-like hydrocarbon group having 1 to 12 carbon atoms (the chain-like hydrocarbon group may have a substituent, and —CH₂— contained in the hydrocarbon group is optionally replaced with —O—, —CO—, —S—, or —SO₂—.).

Examples of the aromatic hydrocarbon groups in Ar¹ include arylene groups such as a phenylene group, a naphthylene group, an anthrylene group, a biphenylene group and a phenanthrylene group. The number of carbon atoms of the aromatic hydrocarbon group is preferably 6 to 16, is more preferably 6 to 14, and is further preferably 6 to 10.

Examples of the substituent other than an iodine atom, which the aromatic hydrocarbon group of Ar¹ may have, include a halogen atom other than an iodine atom, a cyano group, a nitro group, a haloalkyl group having 1 to 6 carbon atoms, an alkyl group having 1 to 12 carbon atoms (where —CH₂— contained in the alkyl group may be replaced with —O—, —S—, —SO₂—, —SO—, —NR⁷—, or —CO—). Specific examples thereof include the same groups as those exemplified in the substituents that W¹ may have.

The number of iodine atoms that Ar¹ has is not particularly limited, but is preferably 1 to 4, and more preferably 1 or 2.

The number of substituents other than an iodine atom, which Ar¹ may have, is not particularly limited, but it is preferably 1 or 2.

When Ar¹ is an aromatic hydrocarbon group having 6 carbon atoms (the aromatic hydrocarbon group has an iodine atom, and may have a substituent other than an iodine atom), the bonding position of A¹ to Ar¹ may be at the o-position, m-position, or p-position relative to the bonding position of L². Among them, A¹ is preferably bonded to the o-position or p-position relative to the bonding position of L², and more preferably bonded to the o-position.

When Ar¹ is an aromatic hydrocarbon group having 6 carbon atoms (the aromatic hydrocarbon group has an iodine atom, and may have a substituent other than an iodine atom), the bonding position of the iodine atom to Ar¹ may be at the o-position, m-position, or p-position relative to the bonding position of L². Among them, at least one iodine atom is preferably bonded to the o-position or m-position relative to the bonding position of L².

X¹ is preferably a single bond, —O—, —CO—, —NR⁷—, a phenylene group optionally having a substituent, or a combined group of these groups, and more preferably a single bond, *—O—** *—CO—** *—CO—**, *—CO—**, *—CO—NR⁷—**, *—NR⁷—CO—**, *—O—CO—NR⁷—**, or *-Ax-Ph-Ay-**.

In the above groups, * and ** represent bonding sites, and * represents the bonding site to the carbon atom to which R⁰ is bonded. Ax and Ay each independently represent one or more types of bonds selected from the group consisting of a single bond, an ether bond, a thioether bond, an amide bond, an ester bond, and a carbonate ester bond. Ph represents a phenylene group optionally having a substituent.

When X¹ is a group represented by *-Ax-Ph-Ay-** in the formula (I), it is preferably a linking group represented by the following formula (X1).

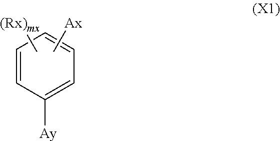

[In formula (X1),
Ax represents a type of bond that bonds to the carbon atom to which R⁰ is bonded, and represents one or more types of bonds selected from the group consisting of a single bond, an ether bond, a thioether bond, an ester bond, an amide bond, and a carbonate ester bond.
Ay represents a type of bond that bonds to L², and represents one or more types of bonds selected from the group consisting of a single bond, an ether bond, a thioether bond, an ester bond, an amide bond, and a carbonate ester bond.
Rx represents a halogen atom, a hydroxy group, a fluorinated alkyl group having 1 to 6 carbon atoms, an alkyl group having 1 to 18 carbon atoms, or an alkoxy group having 1 to 6 carbon atoms; and
mx represents an integer of 0 to 4, and when mx is an integer of 2 or more, a plurality of Rx are the same or different from each other.]

When either Ax or Ay is a single bond, the other is preferably one or more selected from the group consisting of an ether bond, a thioether bond, an ester bond, an amide bond, and a carbonate ester bond.

The bonding position of Ay in the phenylene group is preferably the m-position or p-position with respect to the bonding position of Ax, and more preferably the p-position.

Rx is preferably a fluorine atom, an iodine atom, a trifluoromethyl group, a methyl group, or an ethyl group.
mx is preferably 0, 1, or 2.

Examples of X¹ include a single bond and groups represented by any of the following formulae (X¹-1) to (X¹-10). * and ** represent bonding sites, and * represents the bonding site to the carbon atom to which R⁰ is bonded. X²⁰ represents —O— or —NR⁷—.

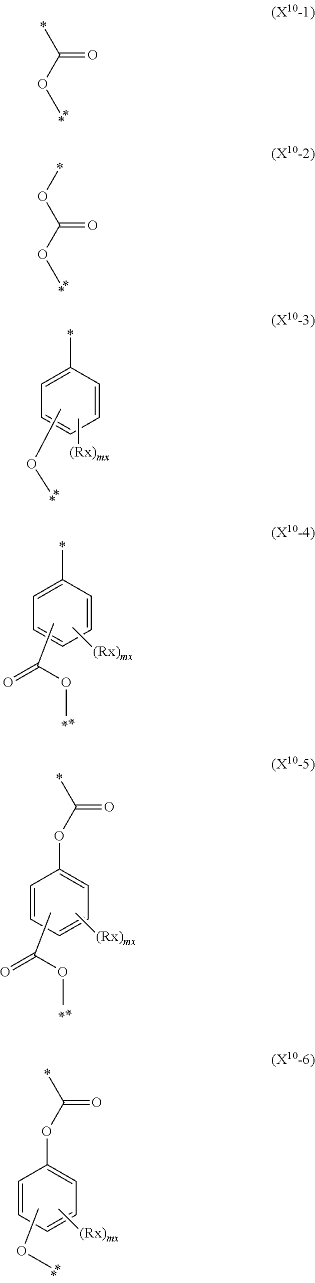

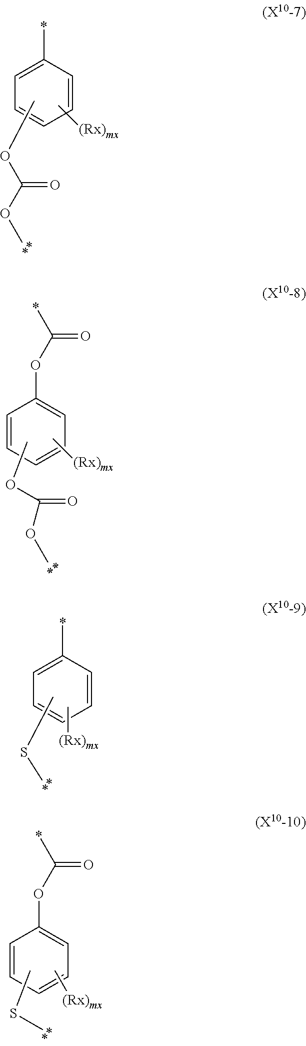

Specific examples of the groups represented by formulas (X¹-1) to (X¹-10) include the following groups.

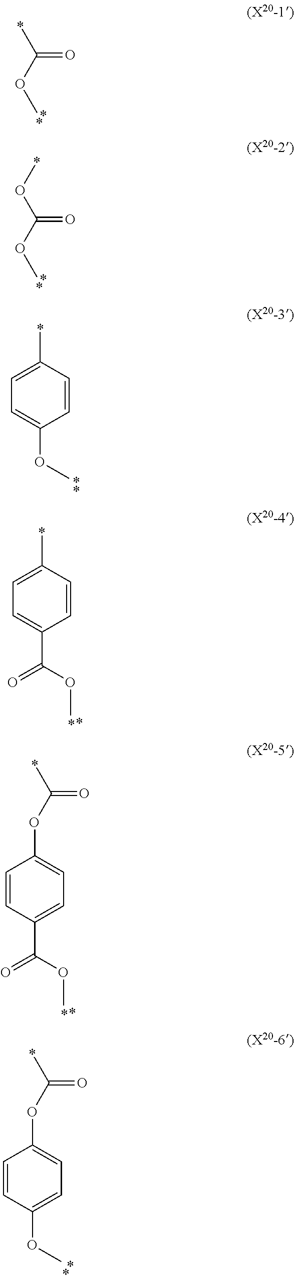

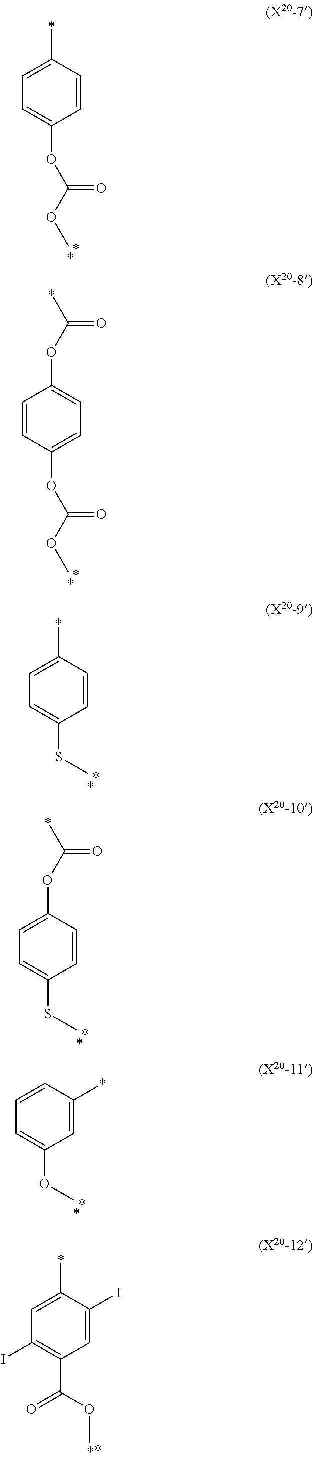

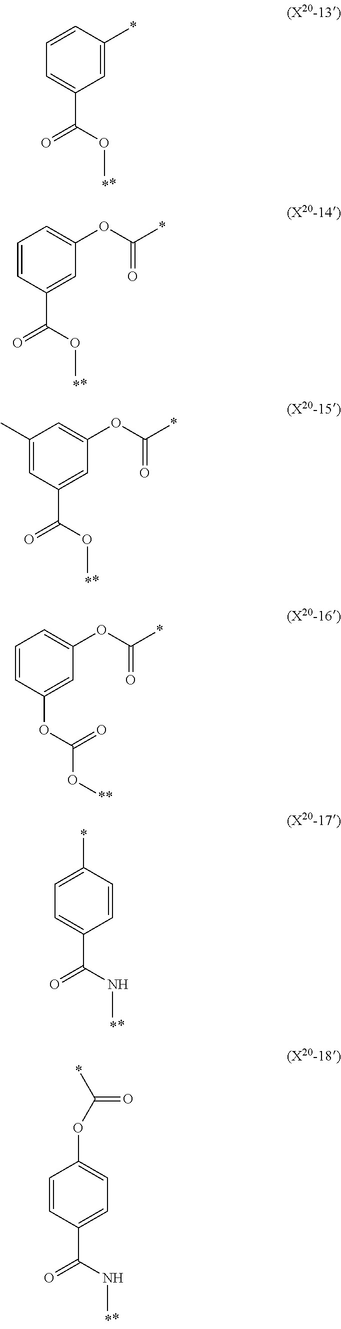

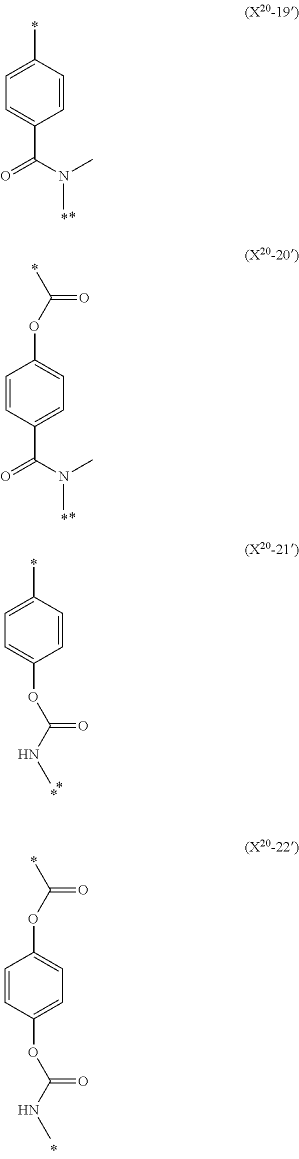 [SMILES: *c1ccc(C(=O)N(C)C)cc1 ; *C(=O)Oc1ccc(C(=O)N(C)C)cc1 ; *c1ccc(OC(=O)NC)cc1 ; *C(=O)Oc1ccc(OC(=O)NC)cc1]

Among these, X¹ is preferably a single bond or a group represented by any one of formulas (X¹-1′), and (X¹-3′) to (X¹-10′), more preferably a single bond or a group represented by any of formulas (X¹-1′), (X¹-4′), (X¹-5′), (X¹-6′), and (X¹-10′), and further preferably a single bond, a group represented by formula (X¹-1′), a group represented by formula (X¹-5′) or a group represented by formula (X¹-6′).

Examples of the alkyl groups of R⁰ include a methyl group, an ethyl group, an n-propyl group, an isopropyl group, an n-butyl group, a sec-butyl group, a tert-butyl group, an n-pentyl group, and an n-hexyl group. It is preferably an alkyl group having 1 to 4 carbon atoms, more preferably an alkyl group having 1 to 3 carbon atoms, and even more preferably a methyl group or an ethyl group. The number of carbon atoms in the alkyl group is preferably 1 to 4, more preferably 1 to 3, and further preferably 1 or 2.

Examples of the halogen atom of R⁰ include a fluorine atom, a chlorine atom, a bromine atom and an iodine atom.

Examples of the alkyl groups having the halogen atom of R⁰ include a fluorinated alkyl group, a chlorinated alkyl group, a brominated alkyl group, and an iodinated alkyl group, and specific examples thereof include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group, a perfluorohexyl group, a perchloromethyl group, a perbromomethyl group, and a periodomethyl group.

R⁰ is preferably a hydrogen atom or an alkyl group having 1 to 4 carbon atoms, more preferably a hydrogen atom or an alkyl group having 1 to 3 carbon atoms, further preferably a hydrogen atom, a methyl group, or an ethyl group, and still more preferably a hydrogen atom or a methyl group.

mm2 is preferably an integer of 1 to 4, is more preferably an integer of 1 to 3, and is further preferably 1 or 2, and is further more preferably 1.

Examples of the anion (I) include anions represented by the following formulae (Ia-1) to (Ia-65).

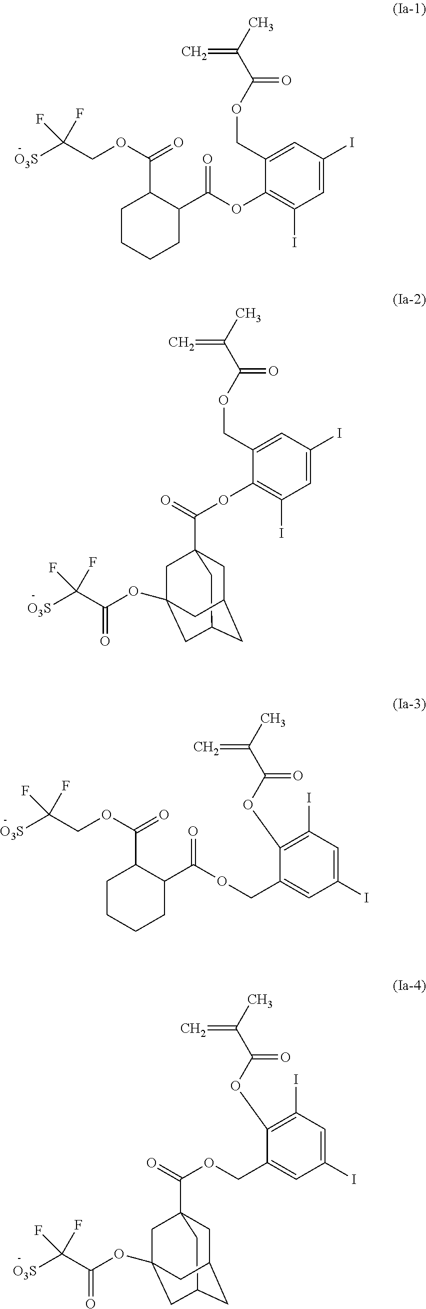

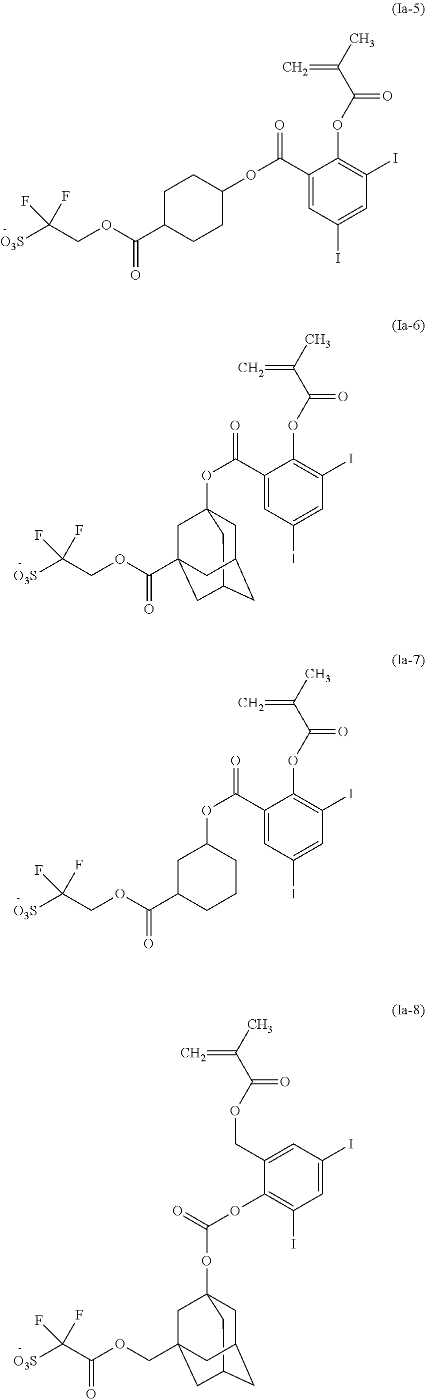

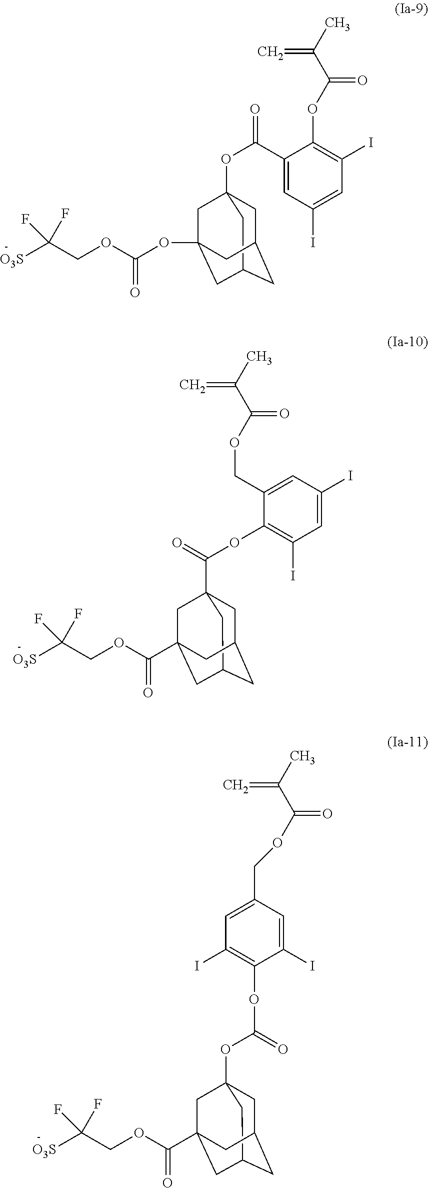

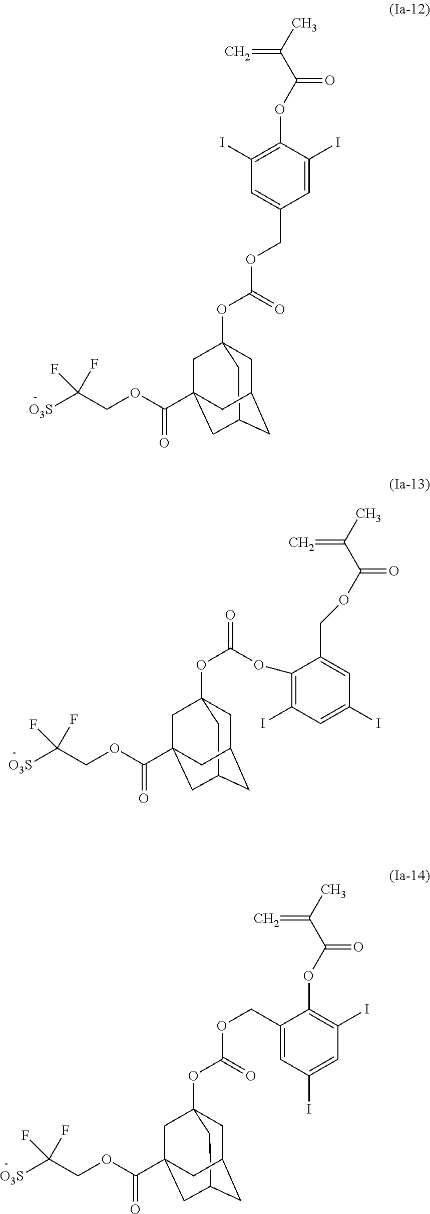

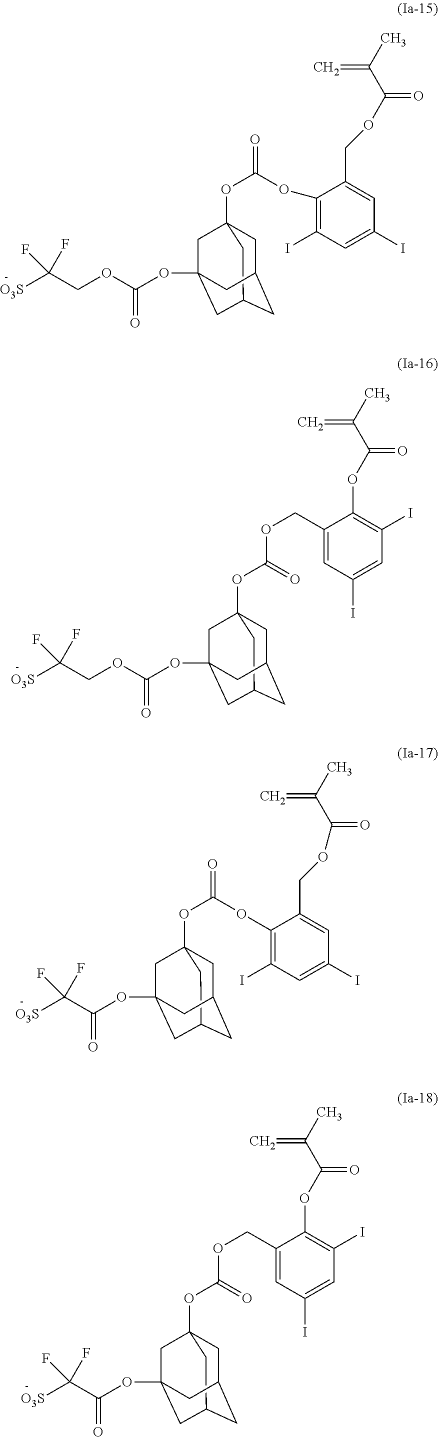

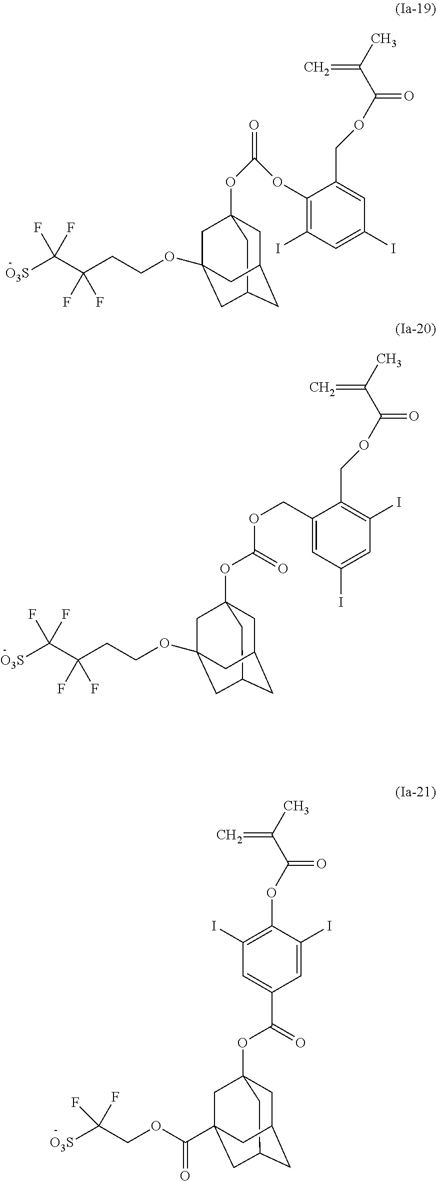

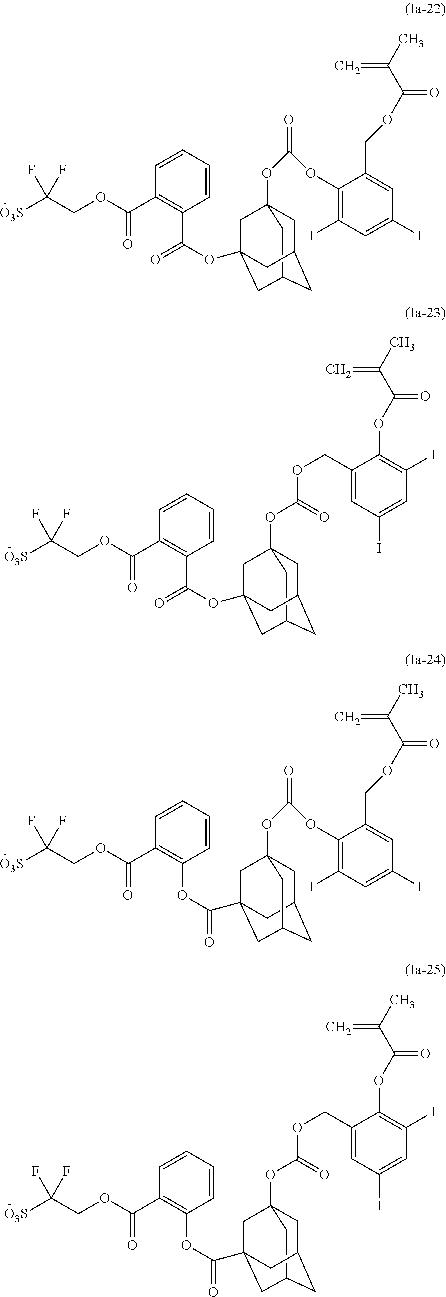

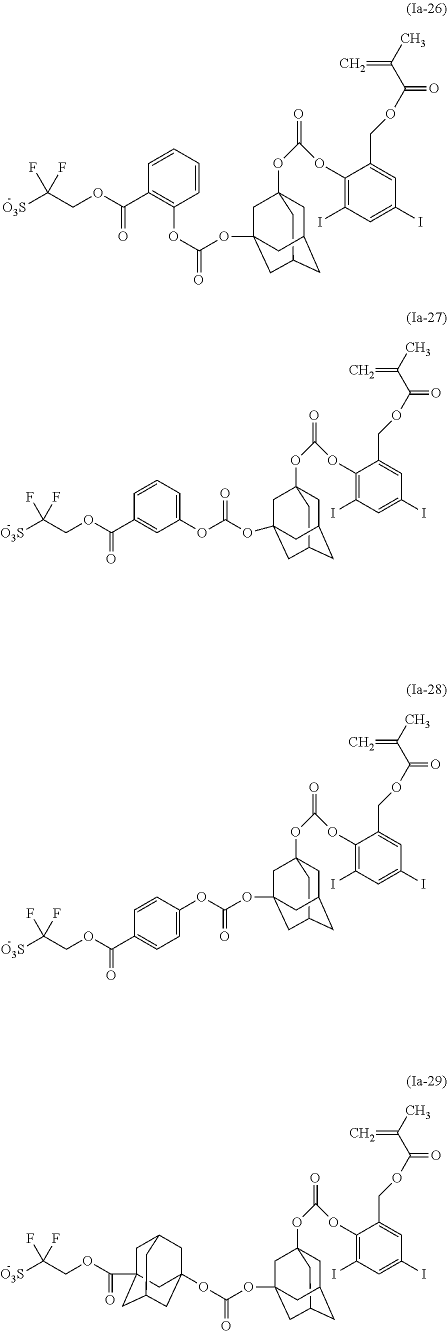

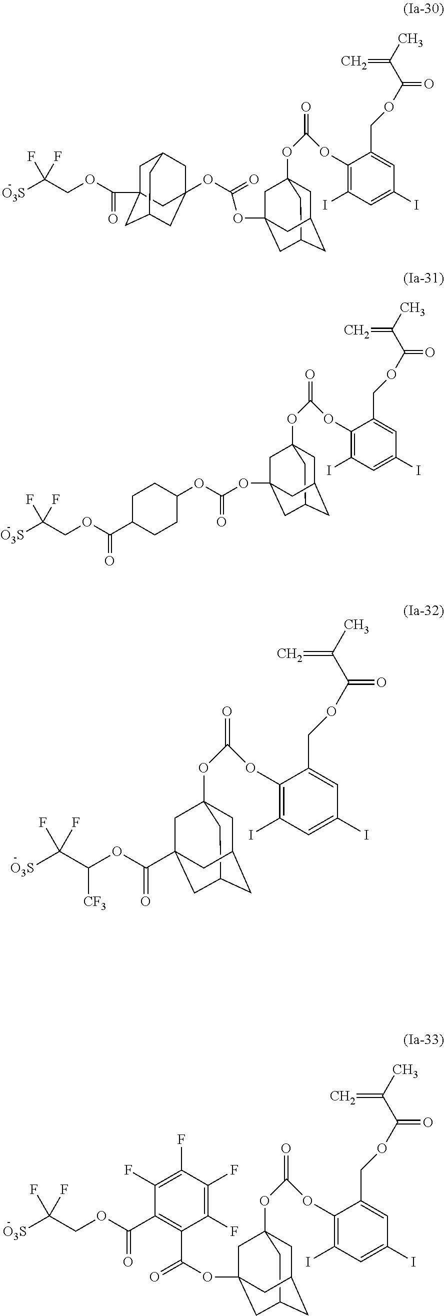

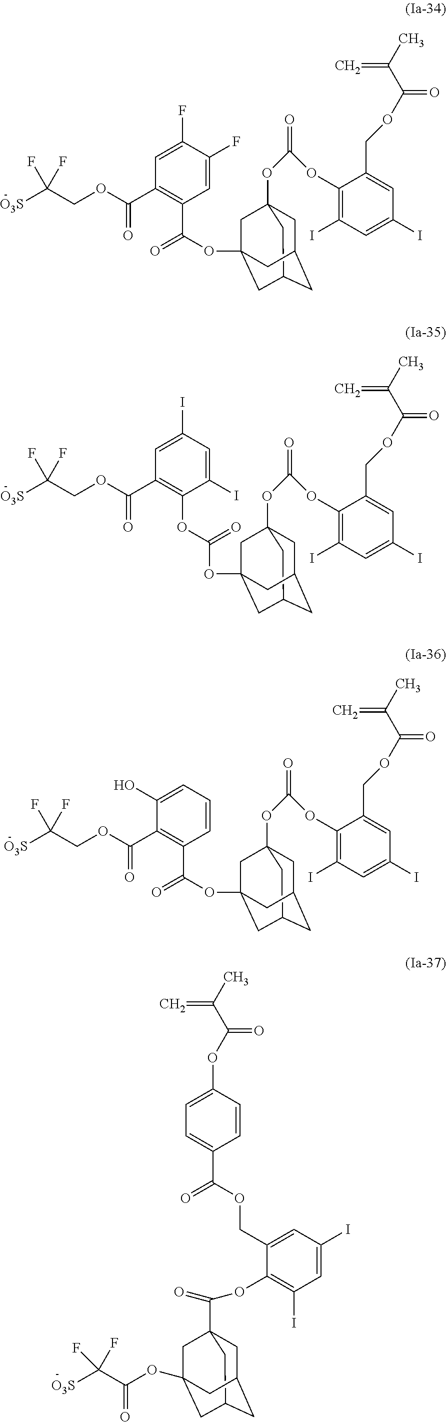

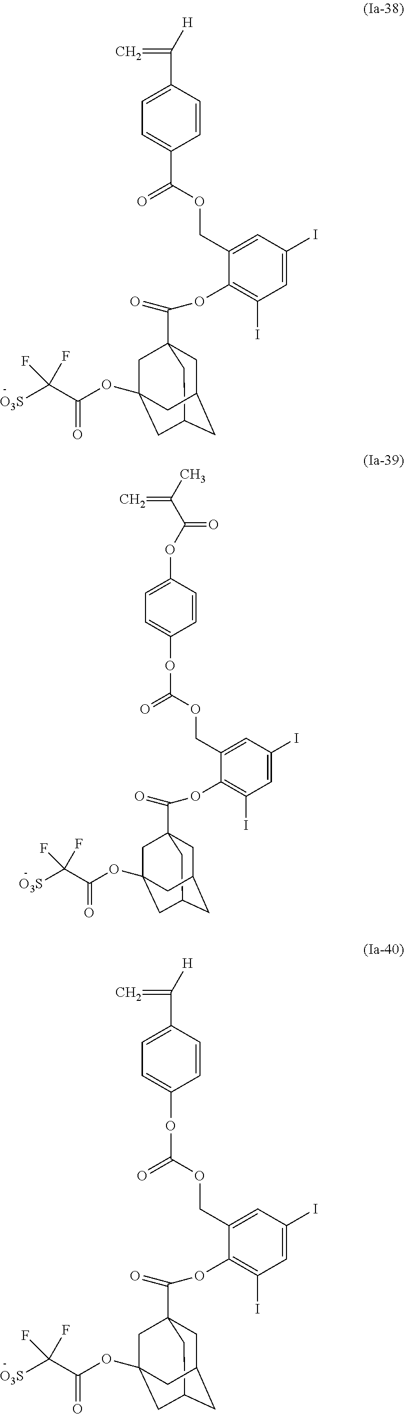

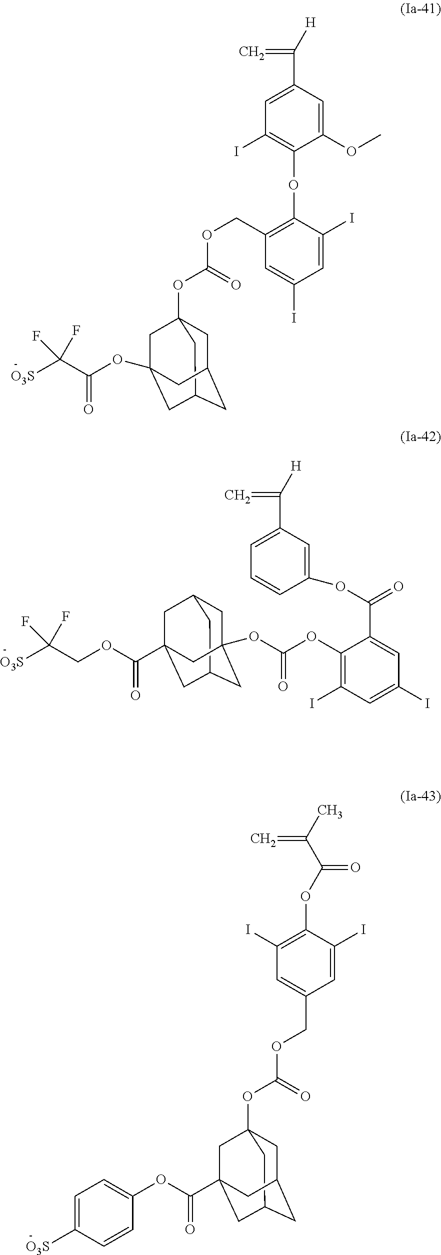

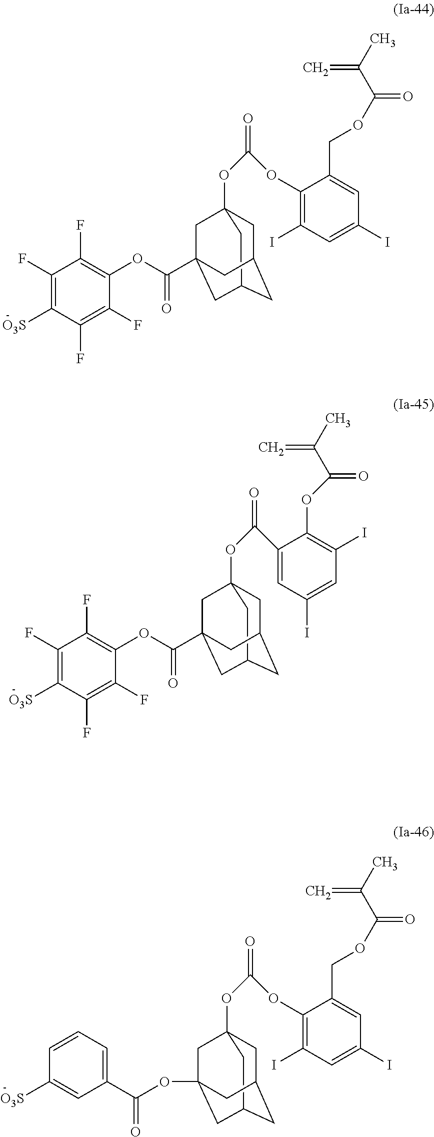

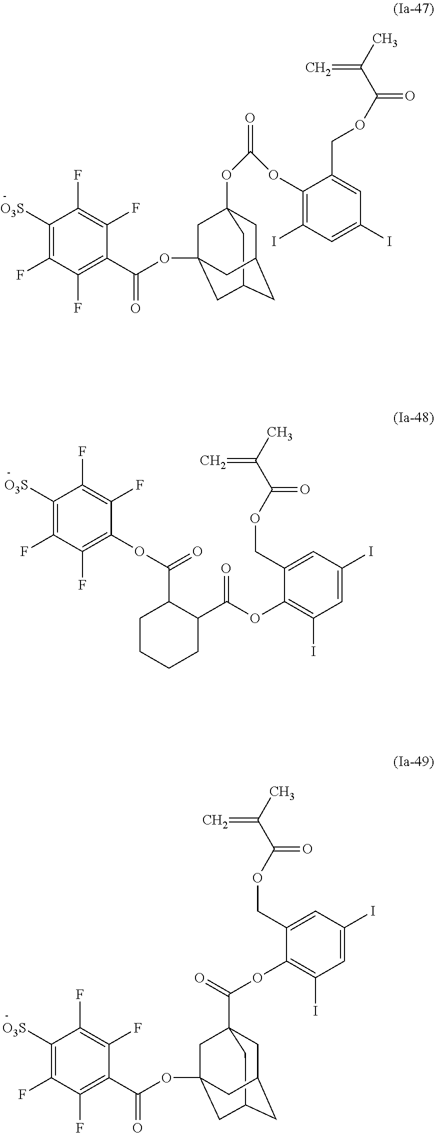

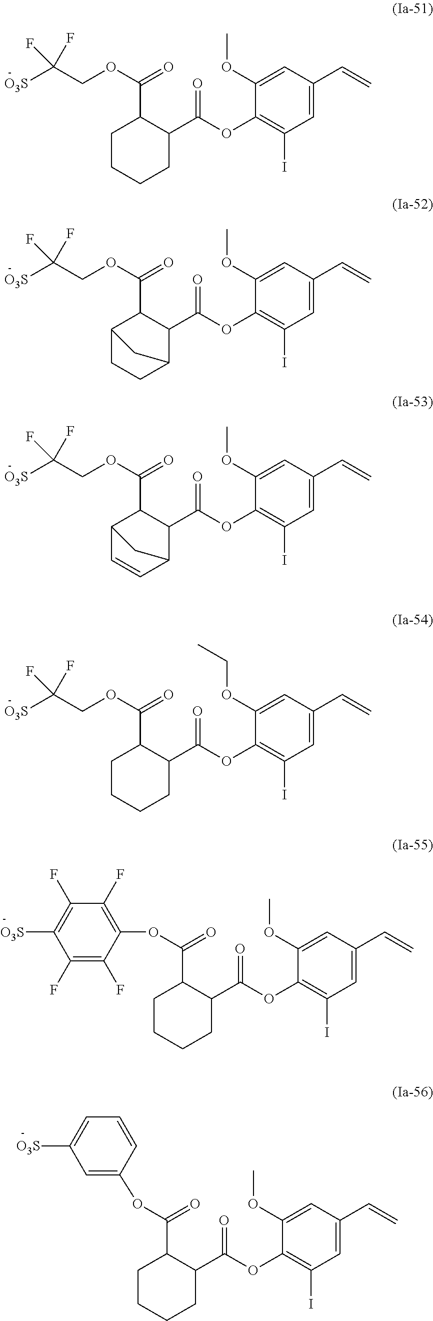

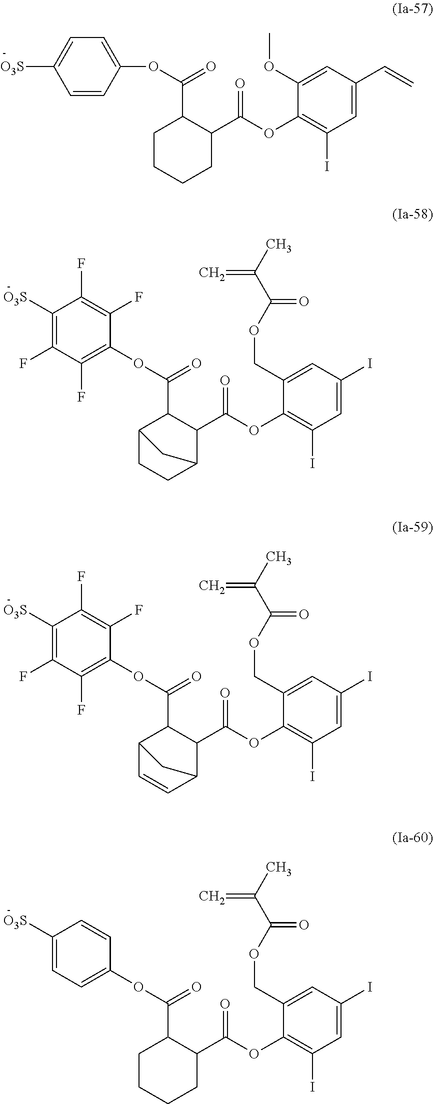

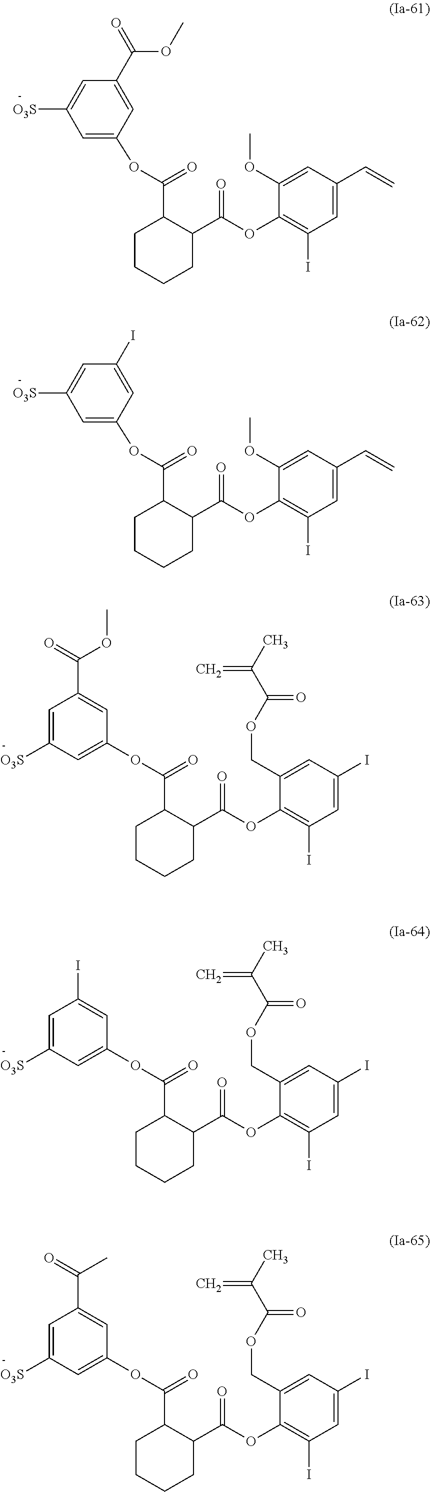

### [Cation (I)]

Examples of the organic cation of Z⁺ in formula (I) include an organic onium cation, an organic sulfonium cation, an organic iodonium cation, an organic ammonium cation, a benzothiazolium cation, and an organic phosphonium cation. Among these cations, an organic sulfonium cation and an organic iodonium cation are preferable, and an arylsulfonium cation is more preferable. Specific examples thereof include cations represented by any of formula (b2-1) to formula (b2-5) (hereinafter referred to as “cation (b2-1)” or the like according to the formula number, in some cases):

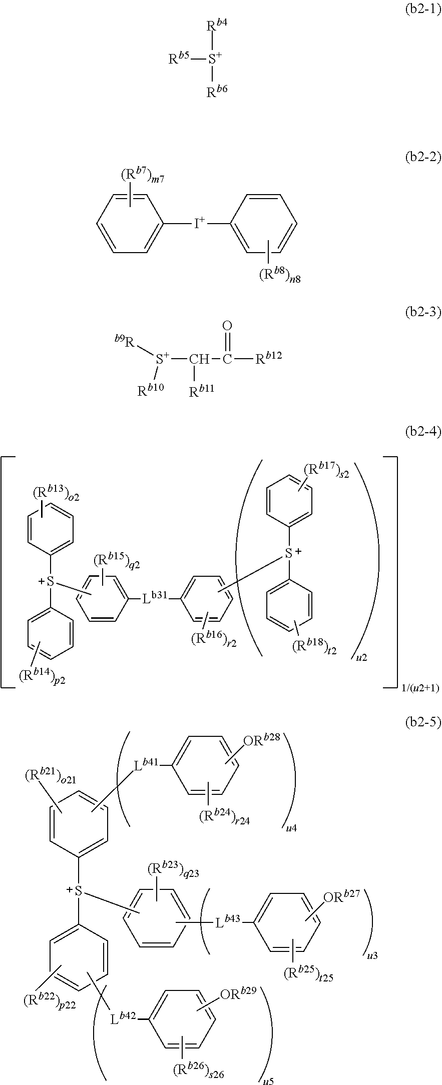

wherein, in the formula (b2-1) to the formula (b2-5),
R^{b4} to R^{b6} each independently represent a chain hydrocarbon group having 1 to 30 carbon atoms, an alicyclic hydrocarbon group having 3 to 36 carbon atoms, or an aromatic hydrocarbon group having 6 to 36 carbon atoms; a hydrogen atom contained in the chain hydrocarbon group may be substituted by a hydroxy group, an alkoxy group having 1 to 12 carbon atoms, an alicyclic hydrocarbon group having 3 to 12 carbon atoms, or an aromatic hydrocarbon group having 6 to 18 carbon atoms; a hydrogen atom contained in the alicyclic hydrocarbon group may be substituted by a halogen atom, an aliphatic hydrocarbon group having 1 to 18 carbon atoms, an alkylcarbonyl group having 2 to 4 carbon atoms, or a glycidyloxy group; and a hydrogen atom contained in the aromatic hydrocarbon group may be substituted by a halogen atom, a hydroxy group, an aliphatic hydrocarbon group having 1 to 18 carbon atoms, a fluorinated alkyl group having 1 to 12 carbon atoms, or an alkoxy group having 1 to 12 carbon atoms.
R^{b4} and R^{b5} may be bonded to each other and form a ring together with the sulfur atom to which R^{b4} and R^{b5} are bonded, and —CH₂— contained in the ring is optionally replaced with —O—, —S—, or —CO—.

R^{b7} and R^{b8} each independently represent a halogen atom, a hydroxy group, an aliphatic hydrocarbon group having 1 to 12 carbon atoms, a fluorinated alkyl group having 1 to 12 carbon atoms, or an alkoxy group having 1 to 12 carbon atoms.

The m7 and n8 each independently represent an integer of 0 to 5.

When the m7 is 2 or larger, a plurality of R^{b7} are the same or different from each other, and when the n8 is 2 or larger, a plurality of R^{b8} may be the same or different from each other.

R^{b9} and R^{b10} each independently represent a chain hydrocarbon group having 1 to 36 carbon atoms or an alicyclic hydrocarbon group having 3 to 36 carbon atoms.

R^{b9} and R^{b10} may be bonded to each other and form a ring together with the sulfur atom to which R^{b9} and R^{b10} are bonded, and —CH₂— contained in the ring is optionally replaced with —O—, —S—, or —CO—.

R^{b12} represents a hydrogen atom, a chain hydrocarbon group having 1 to 36 carbon atoms, an alicyclic hydrocarbon group having 3 to 36 carbon atoms, or an aromatic hydrocarbon group having 6 to 18 carbon atoms.

R^{b12} represents a chain hydrocarbon group having 1 to 12 carbon atoms, an alicyclic hydrocarbon group having 3 to 18 carbon atoms, or an aromatic hydrocarbon group having 6 to 18 carbon atoms; a hydrogen atom contained in the chain hydrocarbon group may be substituted by an aromatic hydrocarbon group having 6 to 18 carbon atoms; and a hydrogen atom contained in the aromatic hydrocarbon group may be substituted by an alkoxy group having 1 to 12 carbon atoms or an alkylcarbonyloxy group having 1 to 12 carbon atoms.

R^{b11} and R^{b12} may be bonded to each other and form a ring including —CH—CO— to which R^{b11} and R^{b12} are bonded, and —CH₂— contained in the ring is optionally replaced with —O—, —S—, or —CO—.

R^{b13} to R^{b18} and R^{b21} to R^{b26} each independently represent a halogen atom, a hydroxy group, an aliphatic hydrocarbon group having 1 to 12 carbon atoms, a fluorinated alkyl group having 1 to 12 carbon atoms, or an alkoxy group having 1 to 12 carbon atoms.

R^{b13} and R^{b14}, and R^{b21} and R^{b22} may be bonded to each other and form a ring containing a sulfur atom together with benzene rings to which R^{b13} and R^{b14}, and R^{b21} and R^{b22} are bonded, and —CH₂— contained in the ring is optionally replaced with —O—, —S— or —CO—.

R^{b27} to R^{b29} each independently represent a hydrogen atom or a group containing an acid labile group.

L^{b31} and L^{b41} to L^{b43} each independently represent a sulfur atom or an oxygen atom.

The o2, p2, s2 and t2 each independently represent an integer of 0 to 5.

The q2 and r2 each independently represent an integer of 0 to 4.

The u2 represents 0 or 1.

When the o2 is 2 or larger, a plurality of R^{b13} are the same or different from each other; when the p2 is 2 or larger, a plurality of R^{b14} are the same or different from each other; when the q2 is 2 or larger, a plurality of R^{b15} are the same or different from each other; when the r2 is 2 or larger, a plurality of R^{b16} are the same or different from each other; when the s2 is 2 or larger, a plurality of R^{b17} are the same or different from each other; and when the t2 is 2 or larger, a plurality of R^{b18} are the same or different from each other.

The u3 represents an integer of 1 to 3, and satisfies 1≤u3+q23≤5.

The u4 and u5 each independently represent an integer of 0 to 3, and satisfy 0≤u4+o21≤5 and 0≤u5+p22≤5.

When the u3, u4 and u5 are each 2 or larger, the groups in a plurality of parentheses are each the same or different.

The o21, p22, q23, r24, t25 and s26 each independently represent an integer of 0 to 4.

When the o21, p22, q23, r24, t25 and s26 are each 2 or larger, a plurality of R^{b21} to R^{b26} are the same or different from each other.

When u2 is 0, any one of o2, p2, q2 and r2 is 1 or larger, and at least one of R^{b21} to R^{b16} is preferably a halogen atom, and when u2 is 1, it is preferable that any one of o2, p2, s2, t2, q2 and r2 is 1 or larger, and at least one of R^{b13} to R^{b18} is a halogen atom.

Furthermore, when u2 is 0, r2 is preferably 1 or larger, and is more preferably 1. In addition, when u2 is 0 and r2 is 1 or larger, R^{b16} is preferably a halogen atom.

The aliphatic hydrocarbon group represents a chain hydrocarbon group and an alicyclic hydrocarbon group.

Examples of the chain hydrocarbon group include alkyl groups such as a methyl group, an ethyl group, a propyl group, an isopropyl group, a butyl group, a sec-butyl group, a tert-butyl group, a pentyl group, a hexyl group, an octyl group and a 2-ethylhexyl group.

In particular, the chain hydrocarbon group of R^{b9} to R^{b12} preferably has 1 to 12 carbon atoms.

The alicyclic hydrocarbon group may be any of a monocyclic type or a polycyclic type, and examples of the monocyclic type of alicyclic hydrocarbon group include cycloalkyl groups such as a cyclopropyl group, a cyclobutyl group, a cyclopentyl group, a cyclohexyl group, a cycloheptyl group, a cyclooctyl group and a cyclodecyl group. Examples of the polycyclic type of alicyclic hydrocarbon group include a decahydronaphthyl group, an adamantyl group, a norbornyl group, and the following groups.

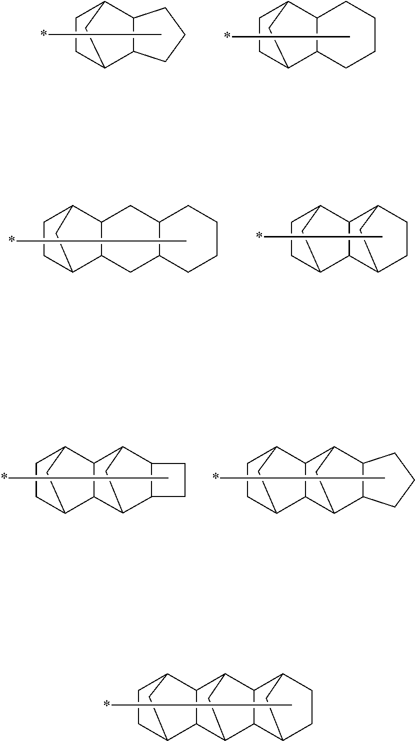

In particular, the alicyclic hydrocarbon groups of R^{b9} to R^{b12} preferably have 3 to 18 carbon atoms, and more preferably have 4 to 12 carbon atoms.

Examples of the alicyclic hydrocarbon group in which a hydrogen atom has been substituted by an aliphatic hydrocarbon group include a methylcyclohexyl group, a dimethylcyclohexyl group, a 2-methyladamantane-2-yl group, a 2-ethyladamantane-2-yl group, a 2-isopropyladamantane-2-yl group, a methylnorbornyl group and an isobornyl group. In the alicyclic hydrocarbon group in which a hydrogen atom has been substituted by an aliphatic hydrocarbon group, the total number of carbon atoms of the alicyclic hydrocarbon group and the aliphatic hydrocarbon group is preferably 20 or smaller.

The fluorinated alkyl group represents an alkyl group having 1 to 12 carbon atoms and a fluorine atom, and examples thereof include a fluoromethyl group, a difluoromethyl group, a trifluoromethyl group and a perfluorobutyl group. The number of carbon atoms of the fluorinated alkyl group is preferably 1 to 9, is more preferably 1 to 6, and is further preferably 1 to 4.

Examples of the aromatic hydrocarbon group include aryl groups such as a phenyl group, a biphenyl group, a naphthyl group and a phenanthryl group. The aromatic hydrocarbon group optionally has a chain hydrocarbon group or an alicyclic hydrocarbon group, and examples thereof include: aromatic hydrocarbon groups having a chain hydrocarbon group (a tolyl group, a xylyl group, a cumenyl group, a mesityl group, a p-ethylphenyl group, a p-tert-butylphenyl group, a 2,6-diethylphenyl group, a 2-methyl-6-ethylphenyl group, and the like); and aromatic hydrocarbon groups having an alicyclic hydrocarbon group (a p-cyclohexylphenyl group, a p-adamantylphenyl group, and the like).

For information, when the aromatic hydrocarbon group has a chain hydrocarbon group or an alicyclic hydrocarbon group, a chain hydrocarbon group having 1 to 18 carbon atoms and an alicyclic hydrocarbon group having 3 to 18 carbon atoms are preferable.

Examples of the aromatic hydrocarbon group in which a hydrogen atom has been substituted by an alkoxy group include a p-methoxyphenyl group.

Examples of the chain hydrocarbon group in which a hydrogen atom has been substituted by an aromatic hydrocarbon group include aralkyl groups such as a benzyl group, a phenethyl group, a phenylpropyl group, a trityl group, a naphthylmethyl group and a naphthylethyl group.

Examples of the alkoxy group include a methoxy group, an ethoxy group, a propoxy group, a butoxy group, a pentyloxy group, a hexyloxy group, a heptyloxy group, an octyloxy group, a decyloxy group and a dodecyloxy group.

Examples of the alkylcarbonyl group include an acetyl group, a propionyl group and a butyryl group.

Examples of the halogen atom include a fluorine atom, a chlorine atom, a bromine atom and an iodine atom.

Examples of the alkylcarbonyloxy group include a methylcarbonyloxy group, an ethylcarbonyloxy group, a propylcarbonyloxy group, an isopropylcarbonyloxy group, a butylcarbonyloxy group, a sec-butylcarbonyloxy group, a tert-butylcarbonyloxy group, a pentylcarbonyloxy group, a hexylcarbonyloxy group, an octylcarbonyloxy group and a 2-ethylhexylcarbonyloxy group.

The ring formed by R^{b4} and R^{b5} that are bonded to each other together with the sulfur atom to which they are bonded may be any ring of a monocyclic type, a polycyclic type, an aromatic type, a non-aromatic type, a saturated type and an unsaturated type. Examples of the ring include a ring having 3 to 18 carbon atoms, and a ring having 4 to 18 carbon atoms is preferable. Examples of the ring containing a sulfur atom include a 3-membered ring to a 12-membered ring; the rings are preferably a 3-membered ring to a 7-membered ring; and examples thereof include the following rings. * represents a bonding site.

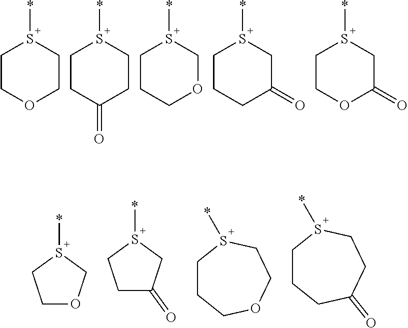

The ring formed by R^{b9} and R^{b0} together may be any ring of a monocyclic type, a polycyclic type, an aromatic type, a non-aromatic type, a saturated type and an unsaturated type. Examples of the ring include a 3-membered ring to a 12-membered ring, and the rings are preferably a 3-membered ring to a 7-membered ring. Examples thereof include a thiolan-1-ium ring (tetrahydrothiophenium ring), a thian-1-ium ring, and a 1,4-oxathian-4-ium ring.

The ring formed by R^{b11} and R^{b12} together may be any ring of a monocyclic type, a polycyclic type, an aromatic type, a non-aromatic type, a saturated type and an unsaturated type. Examples of the ring include a 3-membered ring to a 12-membered ring, and the rings are preferably a 3-membered ring to a 7-membered ring. Examples thereof include an oxocycloheptane ring, an oxocyclohexane ring, an oxonorbornane ring, and an oxoadamantane ring.

Examples of the group containing an acid labile group of R^{b27} to R^{b29} include groups represented by —R^{c30}, —CO—O—R^{c30}, or -L^{c10}-CO—O—R^{c30} (where L^{c10} represents an alkanediyl group having 1 to 6 carbon atoms, and R^{c30} represents an acid labile group).

Examples of the acid labile group of R^{c30} include groups represented by formula (1) or formula (2).

Among the cation (b2-1) to the cation (b2-5), the cation (b2-1), the cation (b2-4) or the cation (b2-5) is preferable.

Examples of the cation (b2-1) include the following cations.

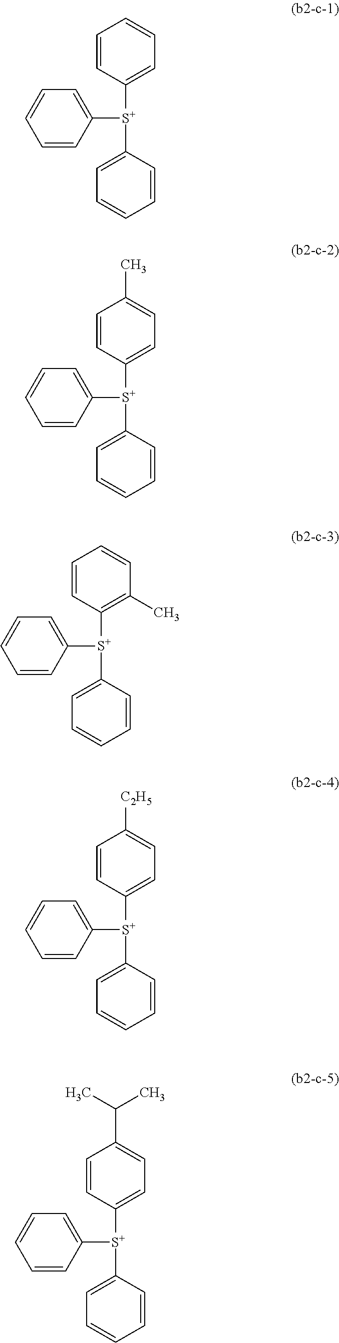 [SMILES: c1ccc([S+](c2ccccc2)c2ccccc2)cc1 ; Cc1ccc([S+](c2ccccc2)c2ccccc2)cc1 ; Cc1ccccc1[S+](c1ccccc1)c1ccccc1 ; CCc1ccc([S+](c2ccccc2)c2ccccc2)cc1 ; CC(C)c1ccc([S+](c2ccccc2)c2ccccc2)cc1]

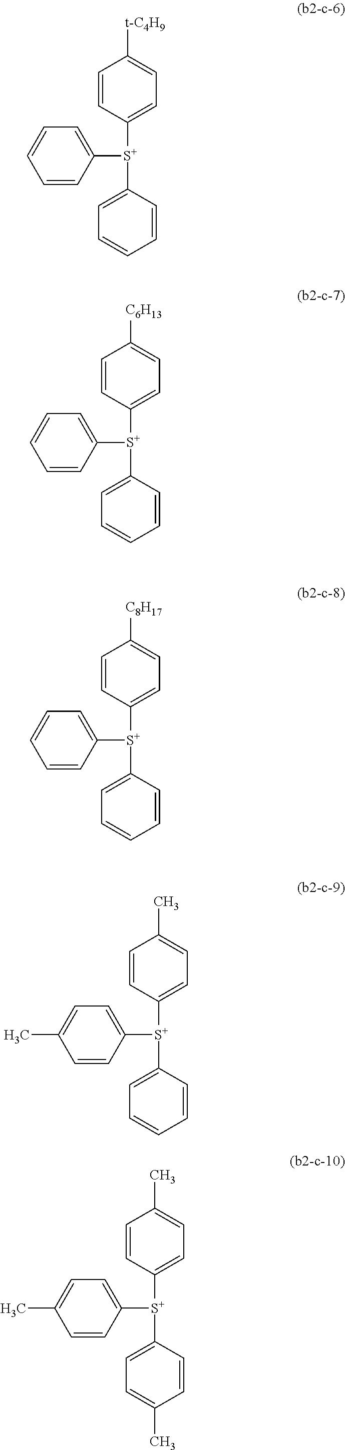

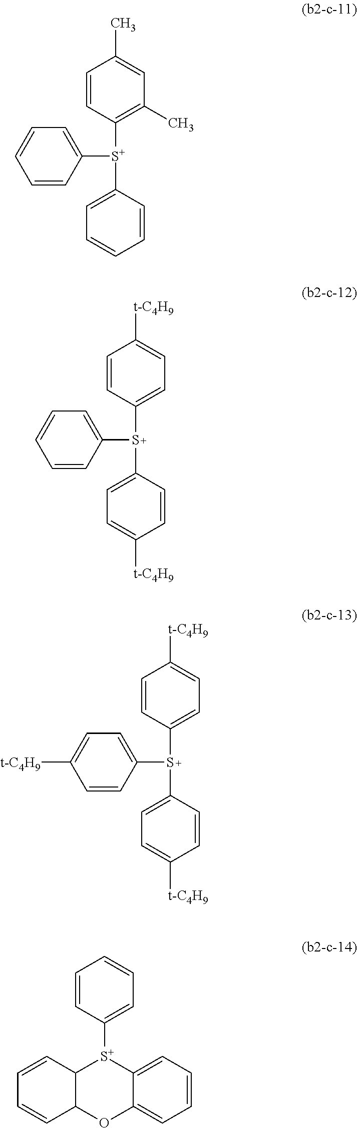

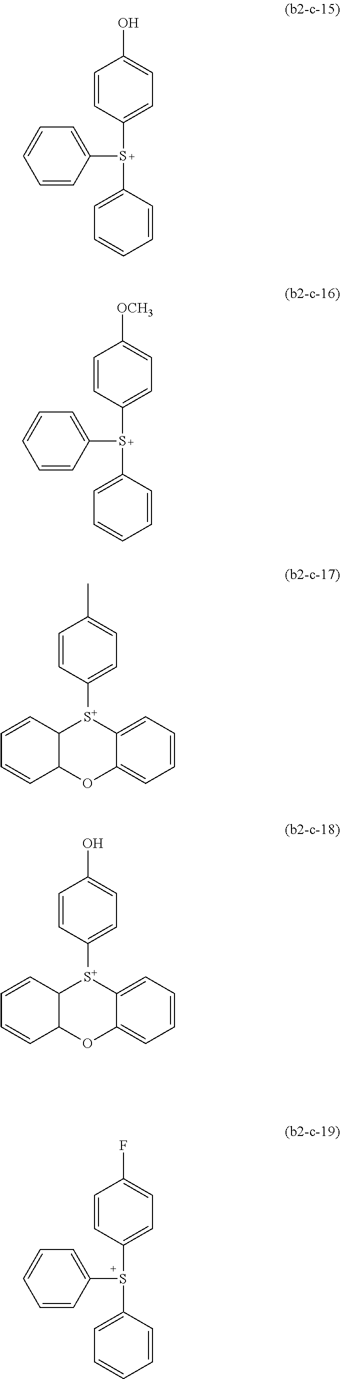

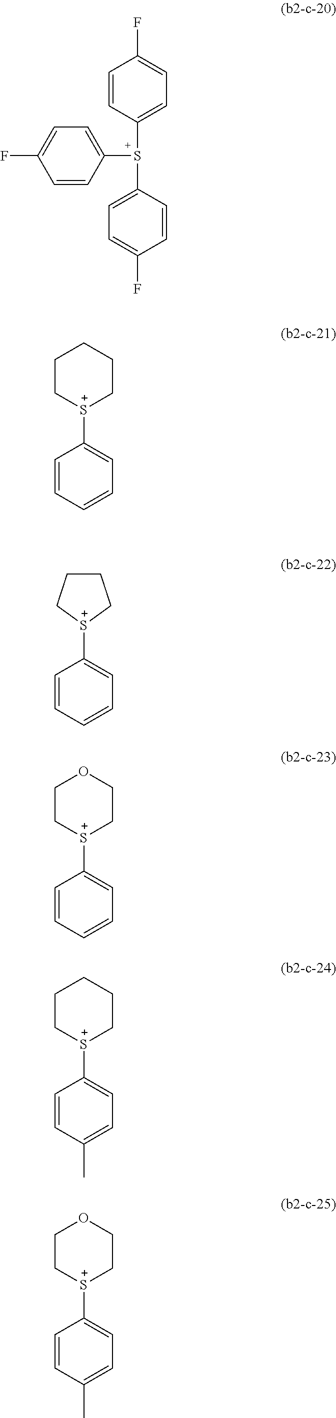

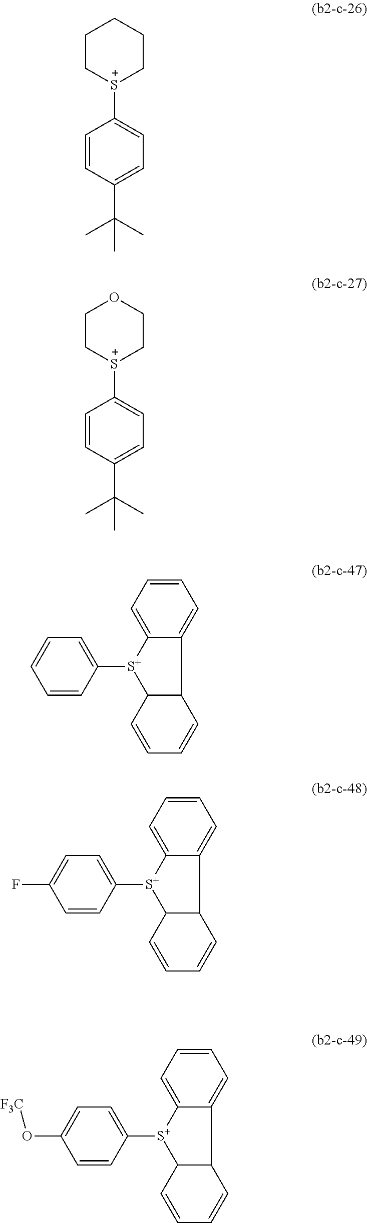 [SMILES: CC(C)(C)c1ccc([S+]2CCCCC2)cc1 ; CC(C)(C)c1ccc([S+]2CCOCC2)cc1 ; C1=CC2c3ccccc3[S+](c3ccccc3)C2C=C1 ; Fc1ccc([S+]2c3ccccc3C3C=CC=CC32)cc1 ; FC(F)(F)Oc1ccc([S+]2c3ccccc3C3C=CC=CC32)cc1]

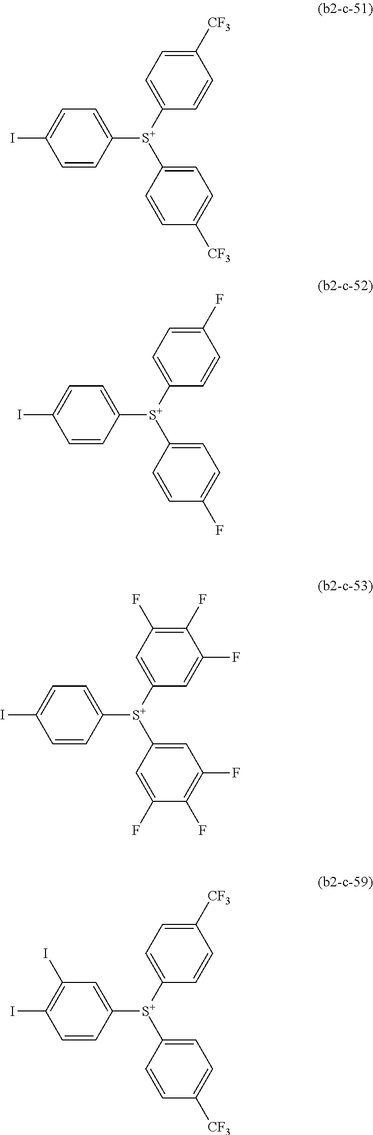 [SMILES: FC(F)(F)c1ccc([S+](c2ccc(I)cc2)c2ccc(C(F)(F)F)cc2)cc1 ; Fc1ccc([S+](c2ccc(F)cc2)c2ccc(I)cc2)cc1 ; Fc1cc([S+](c2ccc(I)cc2)c2cc(F)c(F)c(F)c2)cc(F)c1F ; FC(F)(F)c1ccc([S+](c2ccc(C(F)(F)F)cc2)c2ccc(I)c(I)c2)cc1]

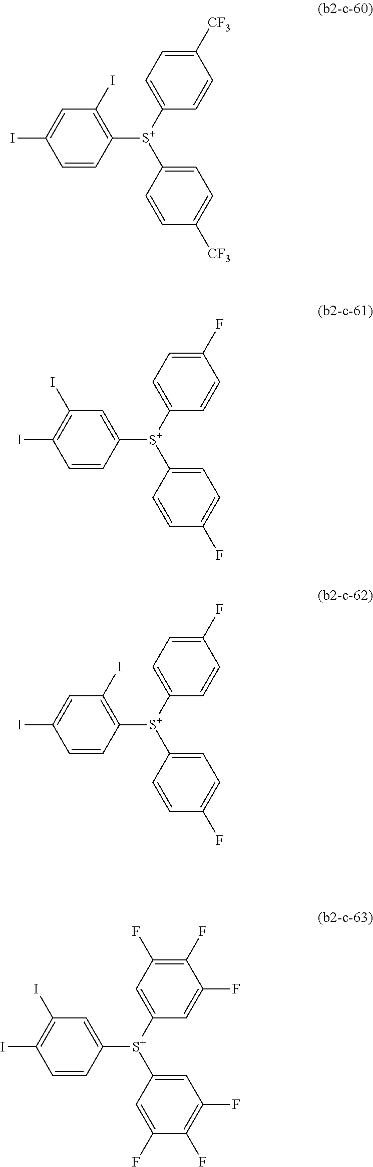 [SMILES: FC(F)(F)c1ccc([S+](c2ccc(C(F)(F)F)cc2)c2ccc(I)cc2I)cc1 ; Fc1ccc([S+](c2ccc(F)cc2)c2ccc(I)c(I)c2)cc1 ; Fc1ccc([S+](c2ccc(F)cc2)c2ccc(I)cc2I)cc1 ; Fc1cc([S+](c2cc(F)c(F)c(F)c2)c2ccc(I)c(I)c2)cc(F)c1F]

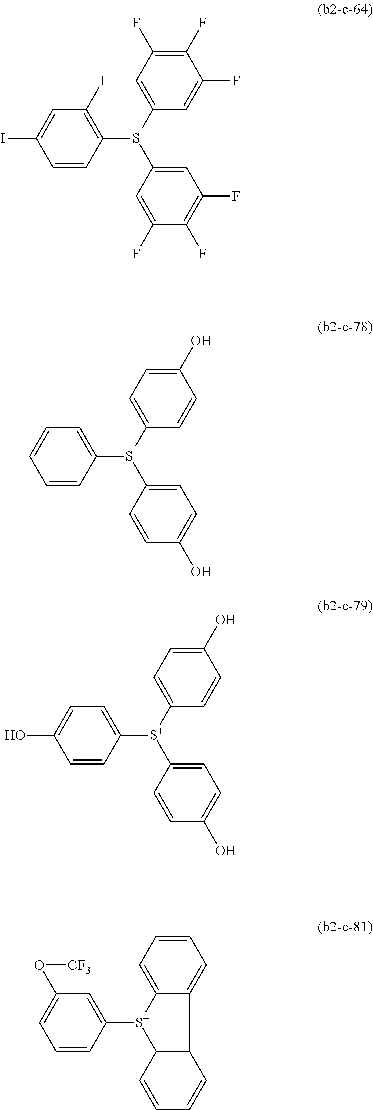 [SMILES: Fc1cc([S+](c2cc(F)c(F)c(F)c2)c2ccc(I)cc2I)cc(F)c1F ; Oc1ccc([S+](c2ccccc2)c2ccc(O)cc2)cc1 ; Oc1ccc([S+](c2ccc(O)cc2)c2ccc(O)cc2)cc1 ; FC(F)(F)Oc1cccc([S+]2c3ccccc3C3C=CC=CC32)c1]

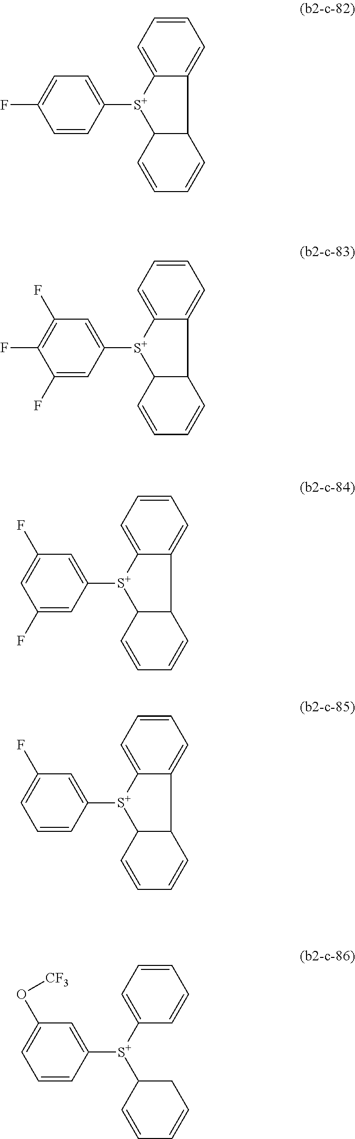 [SMILES: Fc1ccc([S+]2c3ccccc3C3C=CC=CC32)cc1 ; Fc1cc([S+]2c3ccccc3C3C=CC=CC32)cc(F)c1F ; Fc1cc(F)cc([S+]2c3ccccc3C3C=CC=CC32)c1 ; Fc1cccc([S+]2c3ccccc3C3C=CC=CC32)c1 ; FC(F)(F)Oc1cccc([S+](c2ccccc2)C2C=CC=CC2)c1]

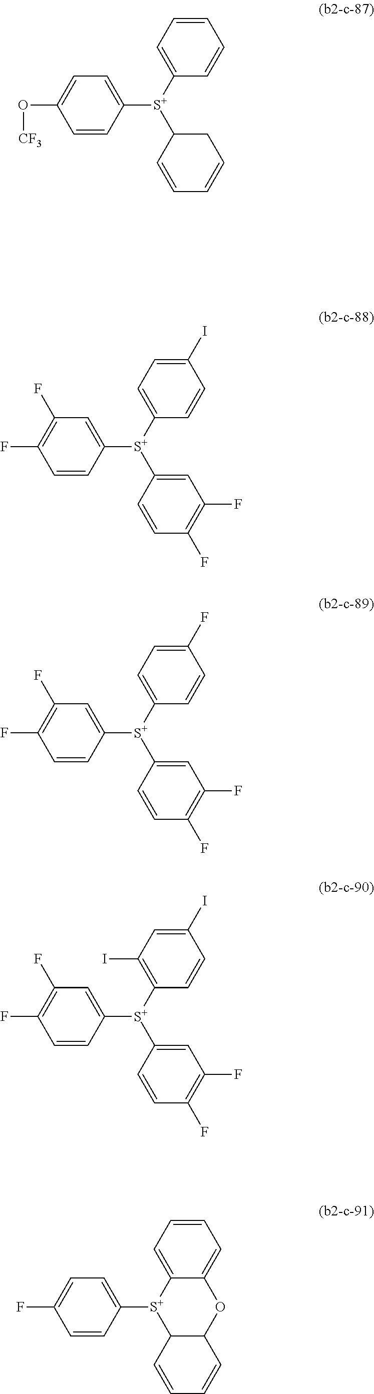 [SMILES: FC(F)(F)Oc1ccc([S+](c2ccccc2)C2C=CC=CC2)cc1 ; Fc1ccc([S+](c2ccc(I)cc2)c2ccc(F)c(F)c2)cc1F ; Fc1ccc([S+](c2ccc(F)c(F)c2)c2ccc(F)c(F)c2)cc1 ; Fc1ccc([S+](c2ccc(F)c(F)c2)c2ccc(I)cc2I)cc1F ; Fc1ccc([S+]2c3ccccc3OC3C=CC=CC32)cc1]

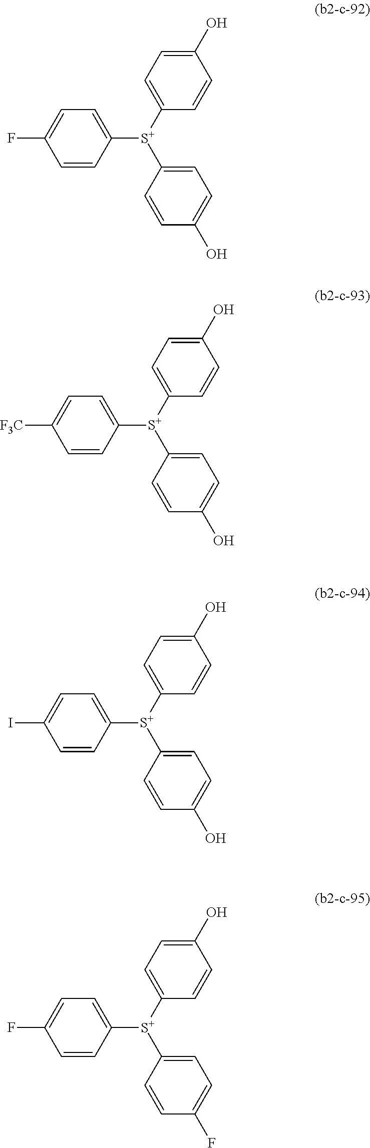 [SMILES: Oc1ccc([S+](c2ccc(O)cc2)c2ccc(F)cc2)cc1 ; Oc1ccc([S+](c2ccc(O)cc2)c2ccc(C(F)(F)F)cc2)cc1 ; Oc1ccc([S+](c2ccc(O)cc2)c2ccc(I)cc2)cc1 ; Oc1ccc([S+](c2ccc(F)cc2)c2ccc(F)cc2)cc1]

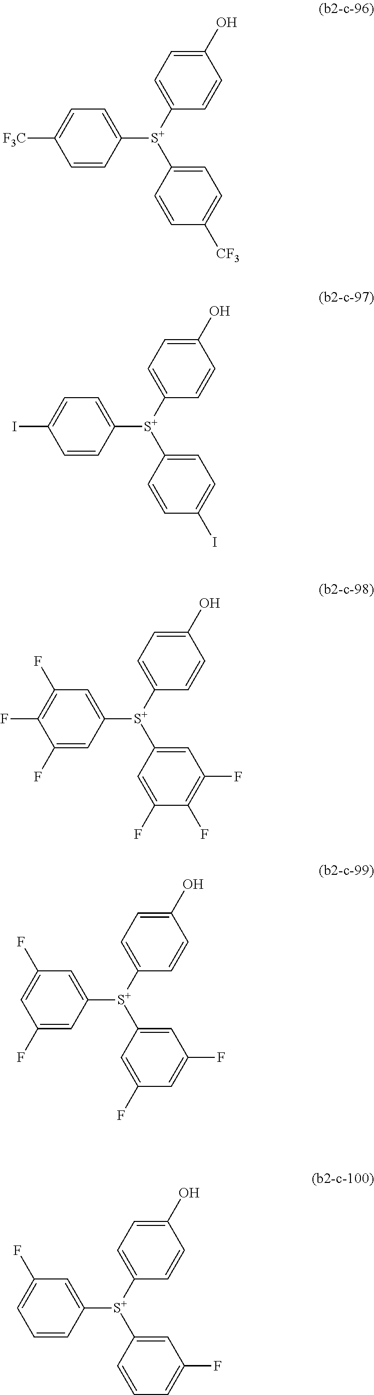 [SMILES: Oc1ccc([S+](c2ccc(C(F)(F)F)cc2)c2ccc(C(F)(F)F)cc2)cc1 ; Oc1ccc([S+](c2ccc(I)cc2)c2ccc(I)cc2)cc1 ; Oc1ccc([S+](c2cc(F)c(F)c(F)c2)c2cc(F)c(F)c(F)c2)cc1 ; Oc1ccc([S+](c2cc(F)cc(F)c2)c2cc(F)cc(F)c2)cc1 ; Oc1ccc([S+](c2cccc(F)c2)c2cccc(F)c2)cc1]

Examples of the cation (b2-2) include the following cations.

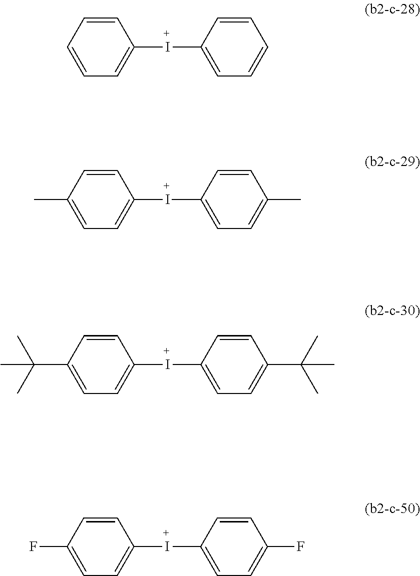

Examples of the cation (b2-3) include the following cations.

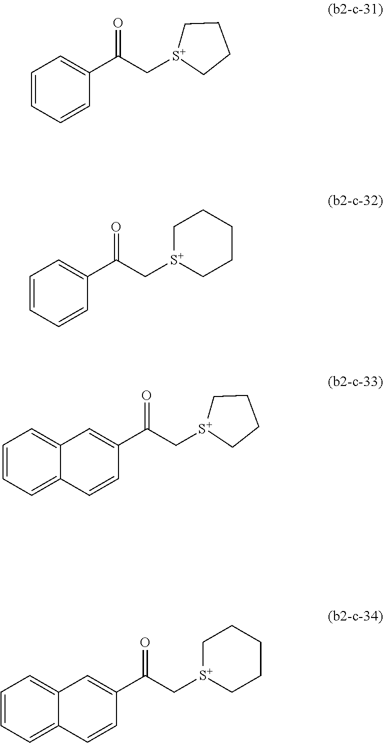 [SMILES: O=C(C[S+]1CCCC1)c1ccccc1 ; O=C(C[S+]1CCCCC1)c1ccccc1 ; O=C(C[S+]1CCCC1)c1ccc2ccccc2c1 ; O=C(C[S+]1CCCCC1)c1ccc2ccccc2c1]

Examples of the cation (b2-4) include the following cations.

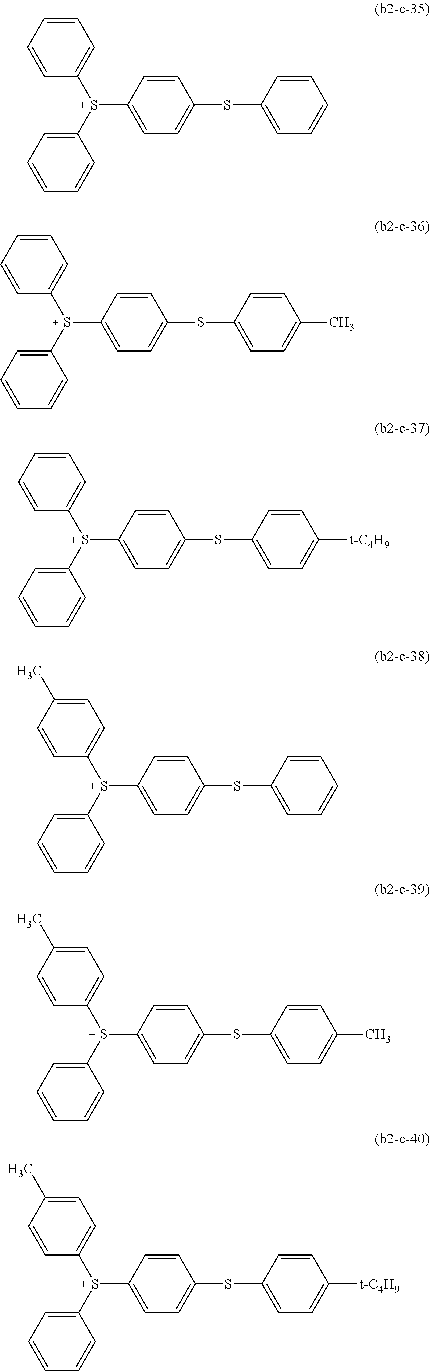

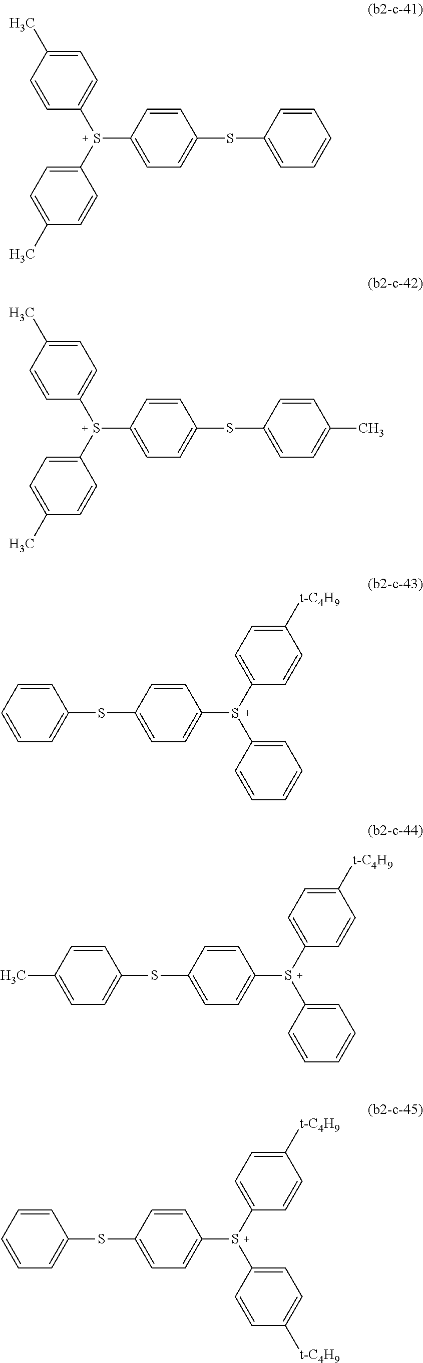

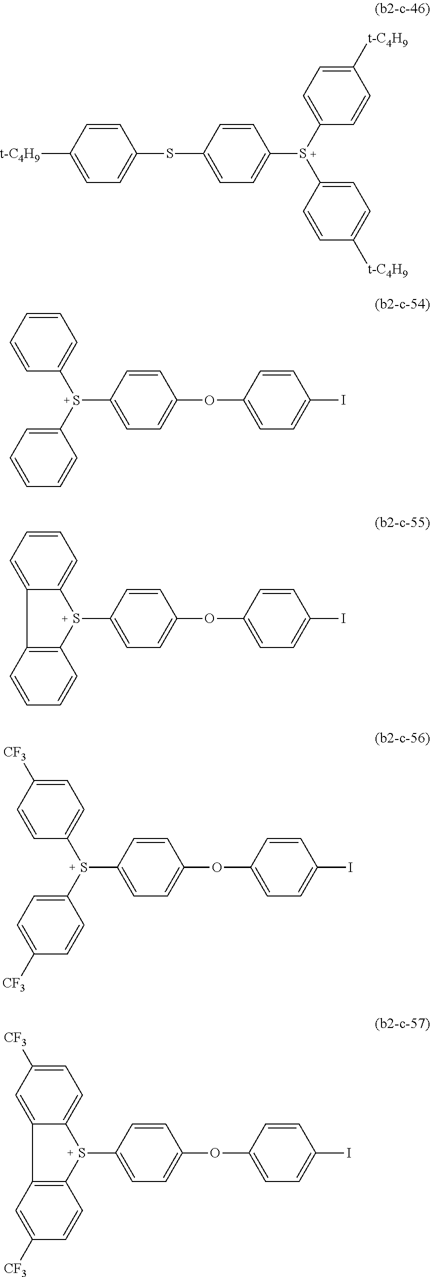

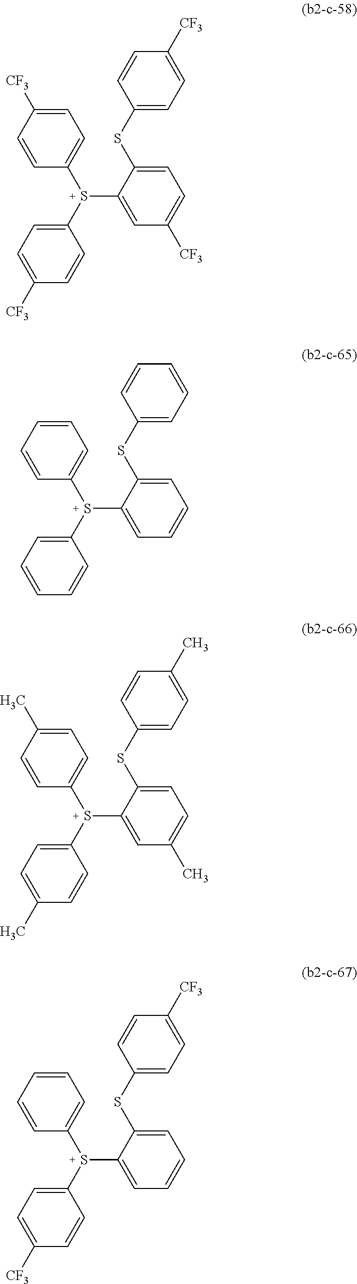

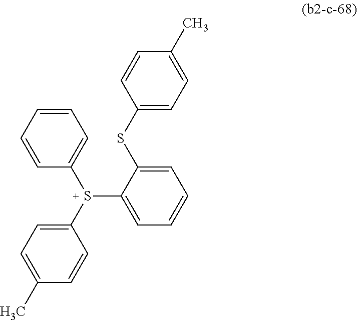

Examples of the cation (b2-5) include the following cations.

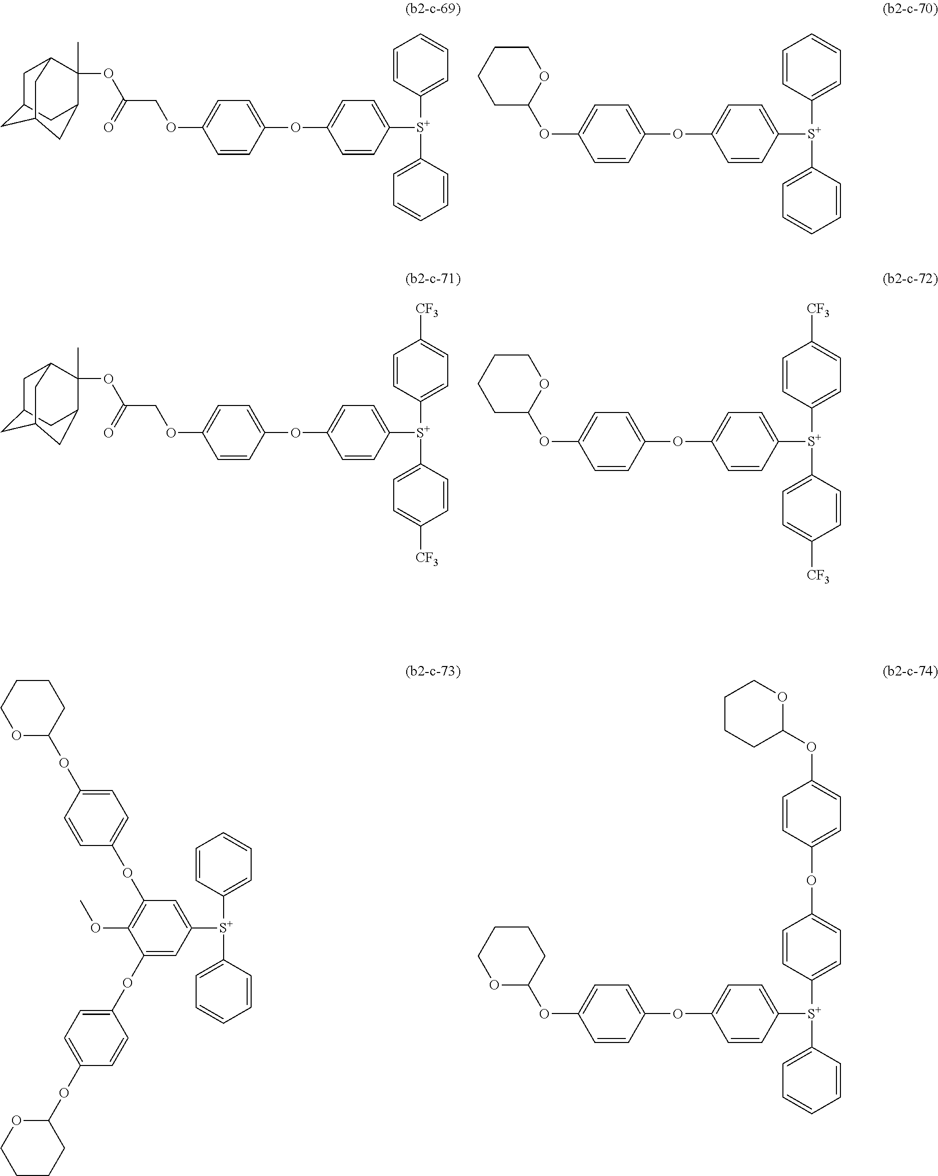 [SMILES: CC1(OC(=O)COc2ccc(Oc3ccc([S+](c4ccccc4)c4ccccc4)cc3)cc2)C2CC3CC(C2)CC1C3 ; c1ccc([S+](c2ccccc2)c2ccc(Oc3ccc(OC4CCCCO4)cc3)cc2)cc1 ; CC1(OC(=O)COc2ccc(Oc3ccc([S+](c4ccc(C(F)(F)F)cc4)c4ccc(C(F)(F)F)cc4)cc3)cc2)C2CC3CC(C2)CC1C3 ; FC(F)(F)c1ccc([S+](c2ccc(Oc3ccc(OC4CCCCO4)cc3)cc2)c2ccc(C(F)(F)F)cc2)cc1 ; COc1c(Oc2ccc(OC3CCCCO3)cc2)cc([S+](c2ccccc2)c2ccccc2)cc1Oc1ccc(OC2CCCCO2)cc1 ; c1ccc([S+](c2ccc(Oc3ccc(OC4CCCCO4)cc3)cc2)c2ccc(Oc3ccc(OC4CCCCO4)cc3)cc2)cc1]

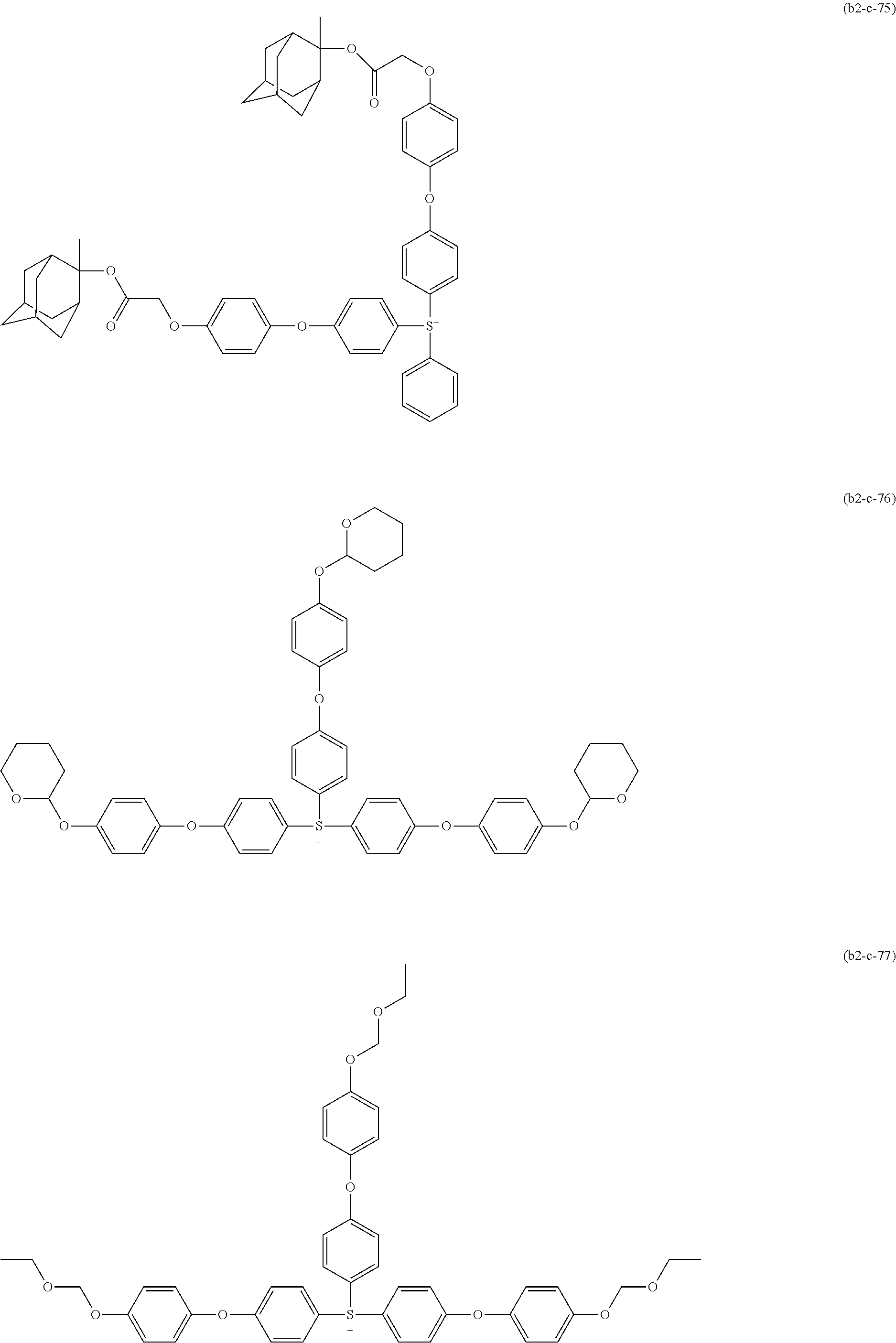

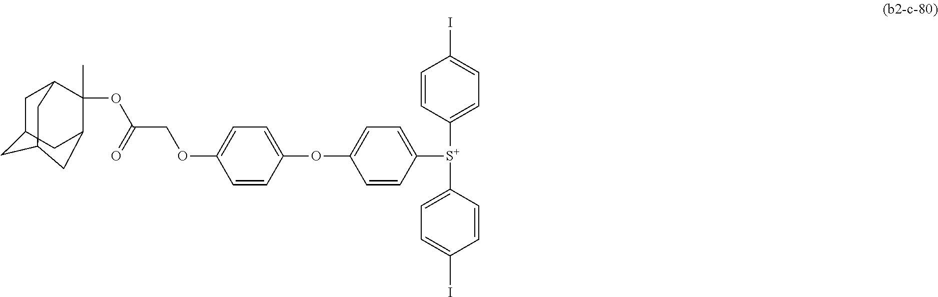 [SMILES: CC1(OC(=O)COc2ccc(Oc3ccc([S+](c4ccc(I)cc4)c4ccc(I)cc4)cc3)cc2)C2CC3CC(C2)CC1C3]

Specific examples of the salt (I) include salts in which the above-described cations and anions are arbitrarily combined. Specific examples of the salt (I) are shown in the following table.

In the following table, each symbol represents a symbol attached to a structure representing the anion or cation described above, and “-” indicates that the salts (I) correspond to the anions (I), respectively. For example, a salt (I-1) means a salt formed of an anion represented by formula (Ia-1) and a cation represented by formula (b2-c-1): a salt (I-2) means a salt formed of an anion represented by formula (Ia-2) and a cation represented by formula (b2-c-1); and a salt (I-51) means a salt formed of an anion represented by formula (Ia-1) and a cation represented by formula (b2-c-2).

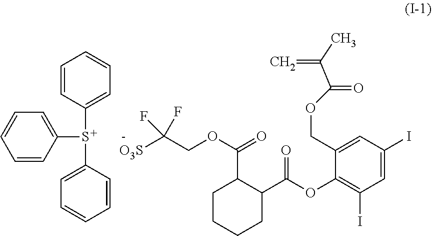

TABLE 1

| Salt (I) | Anion (I) | Cation (I) |
|---|---|---|
| (I-1) to (I-50) | (Ia-1) to (Ia-50) | (b2-c-1) |
| (I-51) to (I-100) | (Ia-1) to (Ia-50) | (b2-c-2) |
| (I-101) to (I-150) | (Ia-1) to (Ia-50) | (b2-c-3) |
| (I-151) to (I-200) | (Ia-1) to (Ia-50) | (b2-c-4) |
| (I-201) to (I-250) | (Ia-1) to (Ia-50) | (b2-c-5) |
| (I-251) to (I-300) | (Ia-1) to (Ia-50) | (b2-c-6) |
| (I-301) to (I-350) | (Ia-1) to (Ia-50) | (b2-c-7) |
| (I-351) to (I-400) | (Ia-1) to (Ia-50) | (b2-c-8) |
| (I-401) to (I-450) | (Ia-1) to (Ia-50) | (b2-c-9) |
| (I-451) to (I-500) | (Ia-1) to (Ia-50) | (b2-c-10) |
| (I-501) to (I-550) | (Ia-1) to (Ia-50) | (b2-c-11) |
| (I-551) to (I-600) | (Ia-1) to (Ia-50) | (b2-c-12) |
| (I-601) to (I-650) | (Ia-1) to (Ia-50) | (b2-c-13) |
| (I-651) to (I-700) | (Ia-1) to (Ia-50) | (b2-c-14) |
| (I-701) to (I-750) | (Ia-1) to (Ia-50) | (b2-c-15) |
| (I-751) to (I-800) | (Ia-1) to (Ia-50) | (b2-c-16) |
| (I-801) to (I-850) | (Ia-1) to (Ia-50) | (b2-c-17) |
| (I-851) to (I-900) | (Ia-1) to (Ia-50) | (b2-c-18) |
| (I-901) to (I-950) | (Ia-1) to (Ia-50) | (b2-c-19) |
| (I-951) to (I-1000) | (Ia-1) to (Ia-50) | (b2-c-20) |
| (I-1001) to (I-1050) | (Ia-1) to (Ia-50) | (b2-c-21) |
| (I-1051) to (I-1100) | (Ia-1) to (Ia-50) | (b2-c-22) |
| (I-1101) to (I-1150) | (Ia-1) to (Ia-50) | (b2-c-23) |
| (I-1151) to (I-1200) | (Ia-1) to (Ia-50) | (b2-c-24) |
| (I-1201) to (I-1250) | (Ia-1) to (Ia-50) | (b2-c-25) |
| (I-1251) to (I-1300) | (Ia-1) to (Ia-50) | (b2-c-26) |
| (I-1301) to (I-1350) | (Ia-1) to (Ia-50) | (b2-c-27) |
| (I-1351) to (I-1400) | (Ia-1) to (Ia-50) | (b2-c-28) |
| (I-1401) to (I-1450) | (Ia-1) to (Ia-50) | (b2-c-29) |
| (I-1451) to (I-1500) | (Ia-1) to (Ia-50) | (b2-c-30) |
| (I-1501) to (I-1550) | (Ia-1) to (Ia-50) | (b2-c-31) |
| (I-1551) to (I-1600) | (Ia-1) to (Ia-50) | (b2-c-32) |
| (I-1601) to (I-1650) | (Ia-1) to (Ia-50) | (b2-c-33) |
| (I-1651) to (I-1700) | (Ia-1) to (Ia-50) | (b2-c-34) |
| (I-1701) to (I-1750) | (Ia-1) to (Ia-50) | (b2-c-35) |
| (I-1751) to (I-1800) | (Ia-1) to (Ia-50) | (b2-c-36) |
| (I-1801) to (I-1850) | (Ia-1) to (Ia-50) | (b2-c-37) |
| (I-1851) to (I-1900) | (Ia-1) to (Ia-50) | (b2-c-38) |
| (I-1901) to (I-1950) | (Ia-1) to (Ia-50) | (b2-c-39) |
| (I-1951) to (I-2000) | (Ia-1) to (Ia-50) | (b2-c-40) |
| (I-2001) to (I-2050) | (Ia-1) to (Ia-50) | (b2-c-41) |
| (I-2051) to (I-2100) | (Ia-1) to (Ia-50) | (b2-c-42) |
| (I-2101) to (I-2150) | (Ia-1) to (Ia-50) | (b2-c-43) |
| (I-2151) to (I-2200) | (Ia-1) to (Ia-50) | (b2-c-44) |
| (I-2201) to (I-2250) | (Ia-1) to (Ia-50) | (b2-c-45) |
| (I-2251) to (I-2300) | (Ia-1) to (Ia-50) | (b2-c-46) |
| (I-2301) to (I-2350) | (Ia-1) to (Ia-50) | (b2-c-47) |
| (I-2351) to (I-2400) | (Ia-1) to (Ia-50) | (b2-c-48) |
| (I-2401) to (I-2450) | (Ia-1) to (Ia-50) | (b2-c-49) |
| (I-2451) to (I-2500) | (Ia-1) to (Ia-50) | (b2-c-50) |
| (I-2501) to (I-2550) | (Ia-1) to (Ia-50) | (b2-c-51) |
| (I-2551) to (I-2600) | (Ia-1) to (Ia-50) | (b2-c-52) |
| (I-2601) to (I-2650) | (Ia-1) to (Ia-50) | (b2-c-53) |
| (I-2651) to (I-2700) | (Ia-1) to (Ia-50) | (b2-c-54) |
| (I-2701) to (I-2750) | (Ia-1) to (Ia-50) | (b2-c-55) |
| (I-2751) to (I-2800) | (Ia-1) to (Ia-50) | (b2-c-56) |
| (I-2801) to (I-2850) | (Ia-1) to (Ia-50) | (b2-c-57) |
| (I-2851) to (I-2900) | (Ia-1) to (Ia-50) | (b2-c-58) |
| (I-2901) to (I-2950) | (Ia-1) to (Ia-50) | (b2-c-59) |
| (I-2951) to (I-3000) | (Ia-1) to (Ia-50) | (b2-c-60) |
| (I-3001) to (I-3050) | (Ia-1) to (Ia-50) | (b2-c-61) |
| (I-3051) to (I-3100) | (Ia-1) to (Ia-50) | (b2-c-62) |
| (I-3101) to (I-3150) | (Ia-1) to (Ia-50) | (b2-c-63) |
| (I-3151) to (I-3200) | (Ia-1) to (Ia-50) | (b2-c-64) |
| (I-3201) to (I-3250) | (Ia-1) to (Ia-50) | (b2-c-65) |
| (I-3251) to (I-3300) | (Ia-1) to (Ia-50) | (b2-c-66) |
| (I-3301) to (I-3350) | (Ia-1) to (Ia-50) | (b2-c-67) |
| (I-3351) to (I-3400) | (Ia-1) to (Ia-50) | (b2-c-68) |
| (I-3401) to (I-3450) | (Ia-1) to (Ia-50) | (b2-c-69) |
| (I-3451) to (I-3500) | (Ia-1) to (Ia-50) | (b2-c-70) |
| (I-3501) to (I-3550) | (Ia-1) to (Ia-50) | (b2-c-71) |
| (I-3551) to (I-3600) | (Ia-1) to (Ia-50) | (b2-c-72) |
| (I-3601) to (I-3650) | (Ia-1) to (Ia-50) | (b2-c-73) |
| (I-3651) to (I-3700) | (Ia-1) to (Ia-50) | (b2-c-74) |
| (I-3701) to (I-3750) | (Ia-1) to (Ia-50) | (b2-c-75) |
| (I-3751) to (I-3800) | (Ia-1) to (Ia-50) | (b2-c-76) |
| (I-3801) to (I-3850) | (Ia-1) to (Ia-50) | (b2-c-77) |
| (I-3851) to (I-3900) | (Ia-1) to (Ia-50) | (b2-c-78) |
| (I-3901) to (I-3950) | (Ia-1) to (Ia-50) | (b2-c-79) |
| (I-3951) to (I-4000) | (Ia-1) to (Ia-50) | (b2-c-80) |
| (I-4001) to (I-4050) | (Ia-1) to (Ia-50) | (b2-c-81) |
| (I-4051) to (I-4100) | (Ia-1) to (Ia-50) | (b2-c-82) |
| (I-4101) to (I-4150) | (Ia-1) to (Ia-50) | (b2-c-83) |
| (I-4151) to (I-4200) | (Ia-1) to (Ia-50) | (b2-c-84) |
| (I-4201) to (I-4250) | (Ia-1) to (Ia-50) | (b2-c-85) |
| (I-4251) to (I-4300) | (Ia-1) to (Ia-50) | (b2-c-86) |
| (I-4301) to (I-4350) | (Ia-1) to (Ia-50) | (b2-c-87) |
| (I-4351) to (I-4400) | (Ia-1) to (Ia-50) | (b2-c-88) |
| (I-4401) to (I-4450) | (Ia-1) to (Ia-50) | (b2-c-89) |
| (I-4451) to (I-4500) | (Ia-1) to (Ia-50) | (b2-c-90) |
| (I-4501) to (I-4550) | (Ia-1) to (Ia-50) | (b2-c-91) |
| (I-4551) to (I-4600) | (Ia-1) to (Ia-50) | (b2-c-92) |
| (I-4601) to (I-4650) | (Ia-1) to (Ia-50) | (b2-c-93) |
| (I-4651) to (I-4700) | (Ia-1) to (Ia-50) | (b2-c-94) |
| (I-4701) to (I-4750) | (Ia-1) to (Ia-50) | (b2-c-95) |
| (I-4751) to (I-4800) | (Ia-1) to (Ia-50) | (b2-c-96) |
| (I-4801) to (I-4850) | (Ia-1) to (Ia-50) | (b2-c-97) |
| (I-4851) to (I-4900) | (Ia-1) to (Ia-50) | (b2-c-98) |
| (I-4901) to (I-4950) | (Ia-1) to (Ia-50) | (b2-c-99) |
| (I-4951) to (I-5000) | (Ia-1) to (Ia-50) | (b2-c-100) |
| (I-5001) to (I-5100) | (Ia-51) | (b2-c-1) to (b2-c-100) |
| (I-5101) to (I-5200) | (Ia-52) | (b2-c-1) to (b2-c-100) |
| (I-5201) to (I-5300) | (Ia-53) | (b2-c-1) to (b2-c-100) |
| (I-5301) to (I-5400) | (Ia-54) | (b2-c-1) to (b2-c-100) |
| (I-5401) to (I-5500) | (Ia-55) | (b2-c-1) to (b2-c-100) |
| (I-5501) to (I-5600) | (Ia-56) | (b2-c-1) to (b2-c-100) |
| (I-5601) to (I-5700) | (Ia-57) | (b2-c-1) to (b2-c-100) |
| (I-5701) to (I-5800) | (Ia-58) | (b2-c-1) to (b2-c-100) |
| (I-5801) to (I-5900) | (Ia-59) | (b2-c-1) to (b2-c-100) |
| (I-5901) to (I-6000) | (Ia-60) | (b2-c-1) to (b2-c-100) |
| (I-6001) to (I-6100) | (Ia-61) | (b2-c-1) to (b2-c-100) |
| (I-6101) to (I-6200) | (Ia-62) | (b2-c-1) to (b2-c-100) |
| (I-6201) to (I-6300) | (Ia-63) | (b2-c-1) to (b2-c-100) |
| (I-6301) to (I-6400) | (Ia-64) | (b2-c-1) to (b2-c-100) |
| (I-6401) to (I-6500) | (Ia-65) | (b2-c-1) to (b2-c-100) |

Among these cations and anions, it is preferable that the salts (I) are salts in which an anion represented by any one of formula (Ia-1) to formula (Ia-65) is combined with a cation represented by any one of formula (b2-c-1), formula (b2-c-10), formula (b2-c-13), formula (b2-c-14), formula (b2-c-18) to formula (b2-c-20), formula (b2-c-27), formula (b2-c-30), formula (b2-c-31), and formula (b2-c-47) to formula (b2-c-100).

### <Method for Producing Salt (I)>

The salt (I) can be produced, for example, by reacting the salt represented by the formula (I-A) with a compound represented by formula (I-B) in a solvent in the presence of a catalyst.

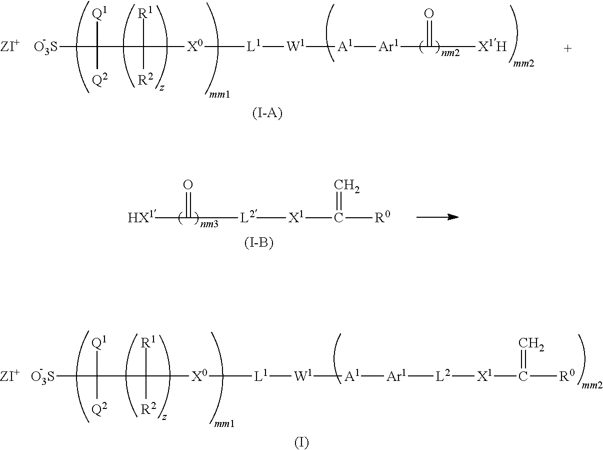

(wherein symbols other than those described below represent the same meanings as described above.

X^{1′} represents —O—, —NR⁷—. nm2 and nm3 each independently represent 0 or 1. L^{2′} represents a group obtained by removing —(CO)ₘₘ₃—X^{1′}—(CO)ₙₘ₂—(CO—X^{1′})ₙₘ₄— from L². However, when nm2=nm3=0, nm4 represents 1, and when either nm2 or nm3 is 1, nm4 represents 0.)

Examples of the solvent in this reaction include tetrahydrofuran, chloroform, and acetonitrile.

Examples of the catalyst include triethylamine, diisopropylethylamine, pyridine, dimethylaminopyridine, carbonyldiimidazole, sodium hydroxide, potassium hydroxide, and 1-[3-(dimethylamino)propyl]-3-ethylcarbodiimide hydrochloride.

A reaction temperature is usually 0° C. to 120° C., and a reaction time period is usually 0.5 to 24 hours.

Examples of the compound represented by the formula (I-B) include the compounds represented by the following formulas, which are easily available on the market or can also be easily produced by a known production method.

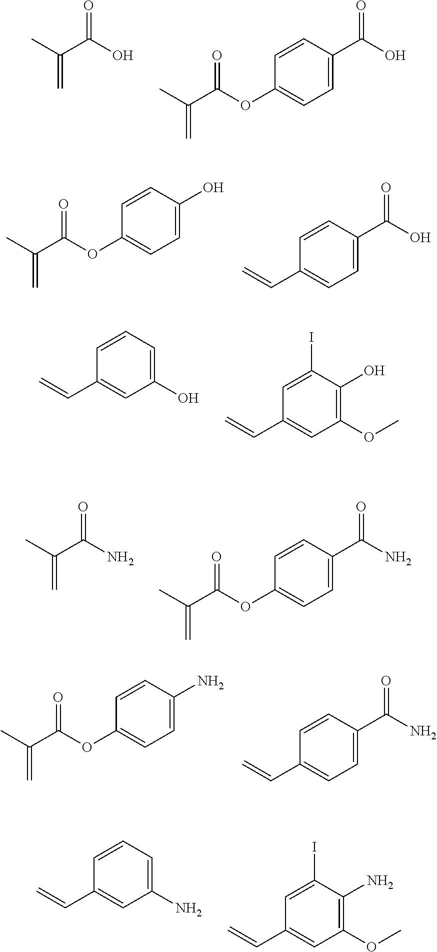

The salt (I-A) can be produced, for example, by reacting the salt represented by the formula (I-a1) with a compound represented by formula (I-b1) or a compound represented by formula (I-b2) in a solvent in the presence of a catalyst, followed by an acid treatment:

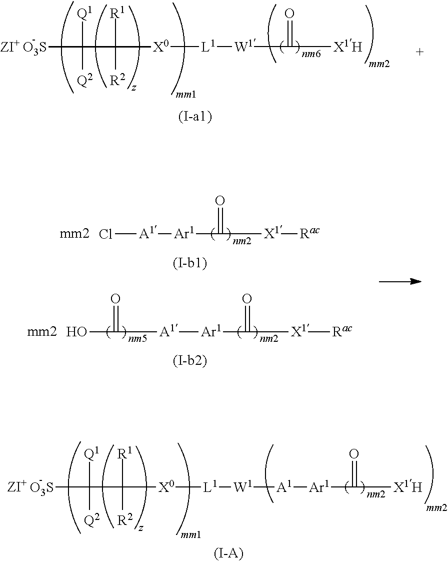

(wherein symbols other than those described below represent the same meanings as described above.

nm5 and nm6 each independently represent 0 or 1. A^{1′} represents a group obtained by removing —(CO)ₙₘ₅—X^{1′}—(CO)ₙₘ₆—(CO—X^{1′})ₙₘ₇— from A¹. However, when nm5=nm6=0, nm7 represents 1, and when either nm5 or nm6 is 1, nm7 represents 0. R^{ac} represents an acid labile group.)

Examples of the solvent in this reaction include chloroform and acetonitrile.

Examples of the catalyst include carbonyl diimidazole and 1-[3-(dimethylamino)propyl]-3-ethylcarbodiimide hydrochloride.

Examples of the acid in this reaction include hydrochloric acid.

A reaction temperature is usually 5° C. to 80° C., and a reaction time period is usually 0.5 to 24 hours.

Examples of the salt represented by the formula (I-a1) include the following salts which are easily available on the market or can also be easily produced by a known production method.

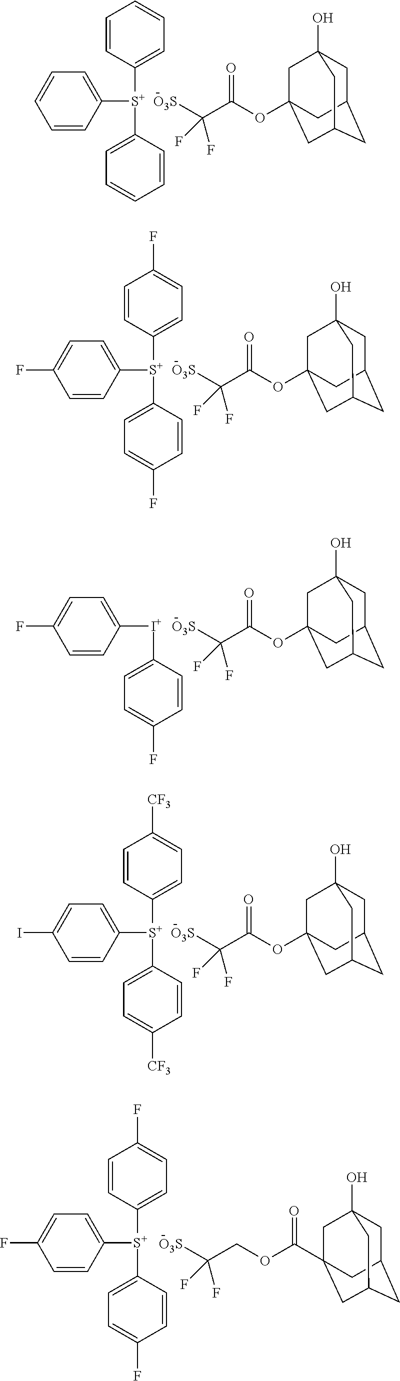

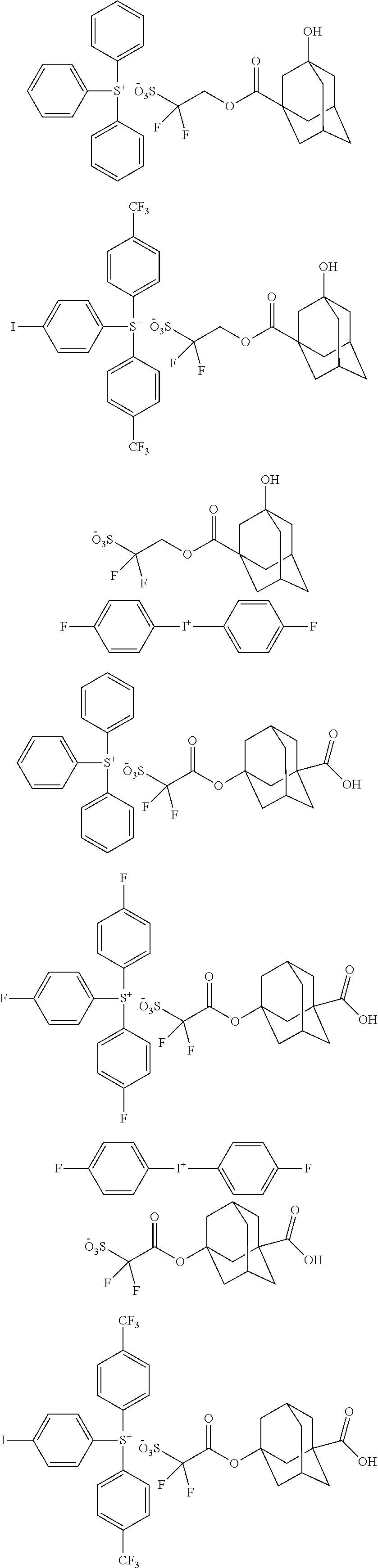

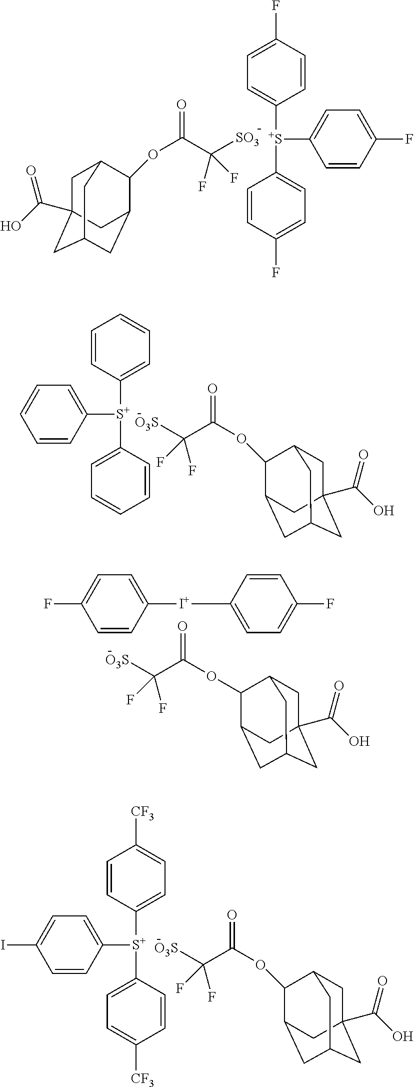

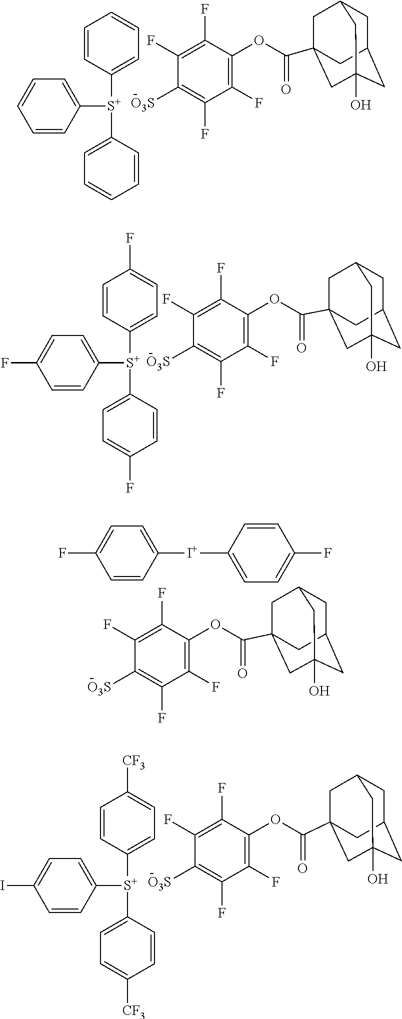

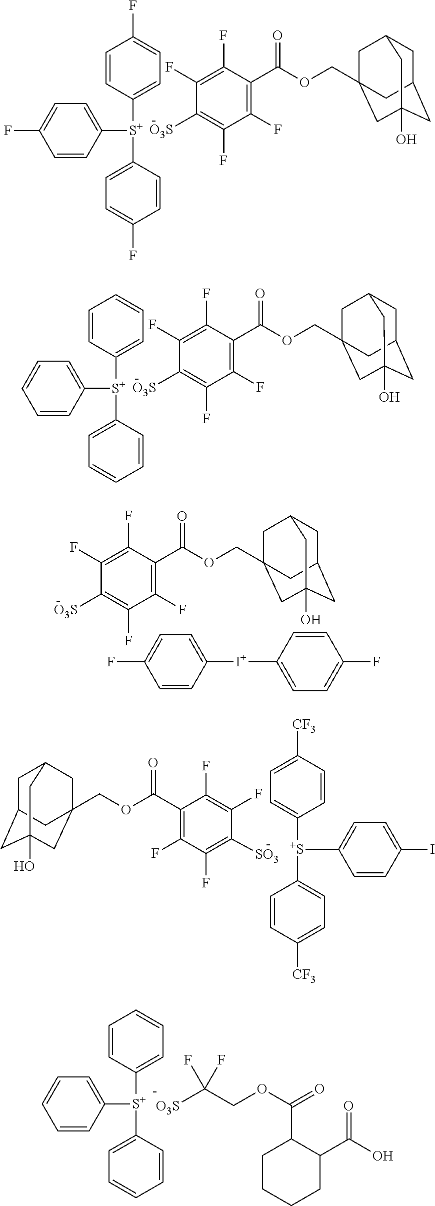

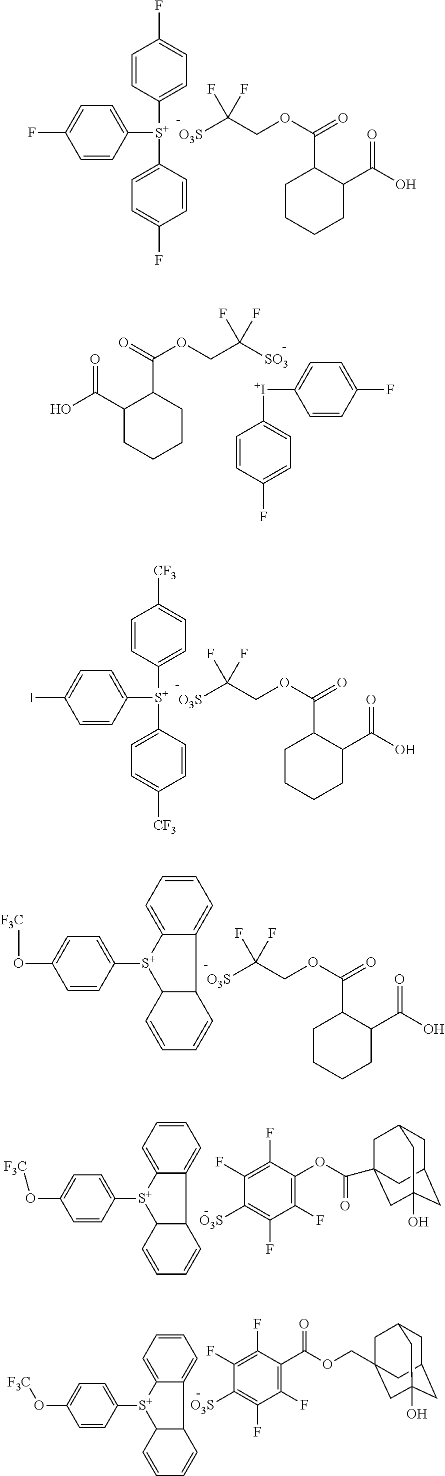

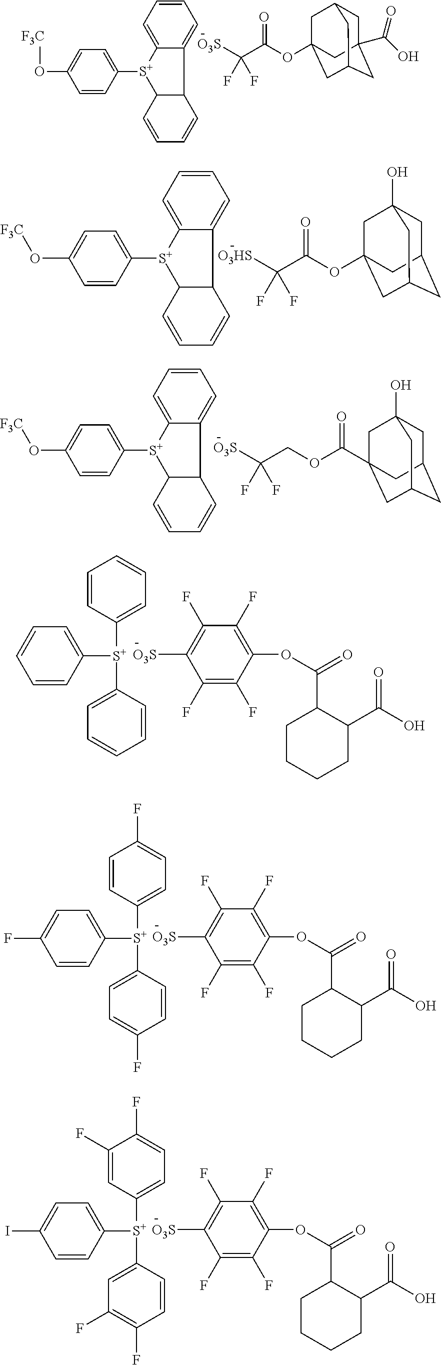

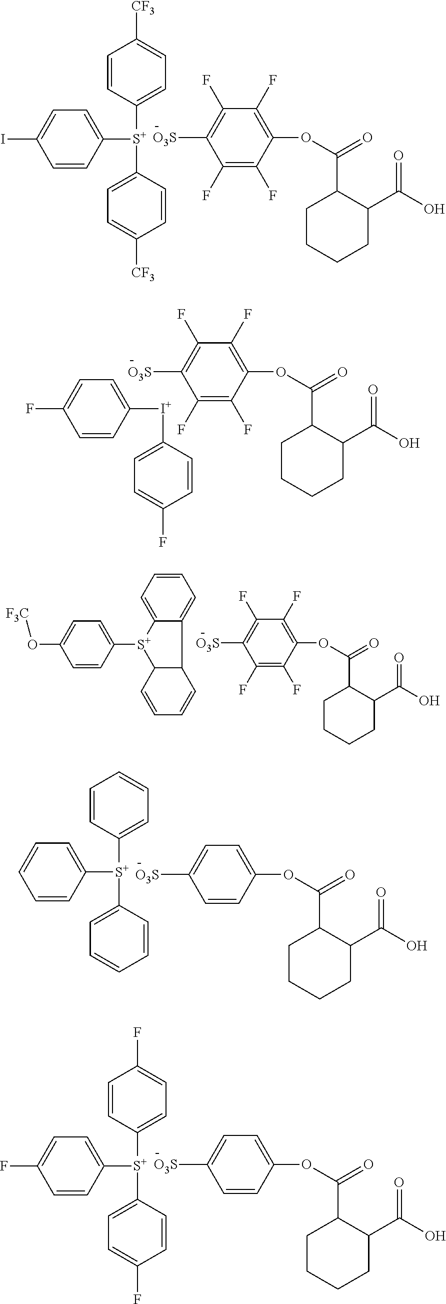

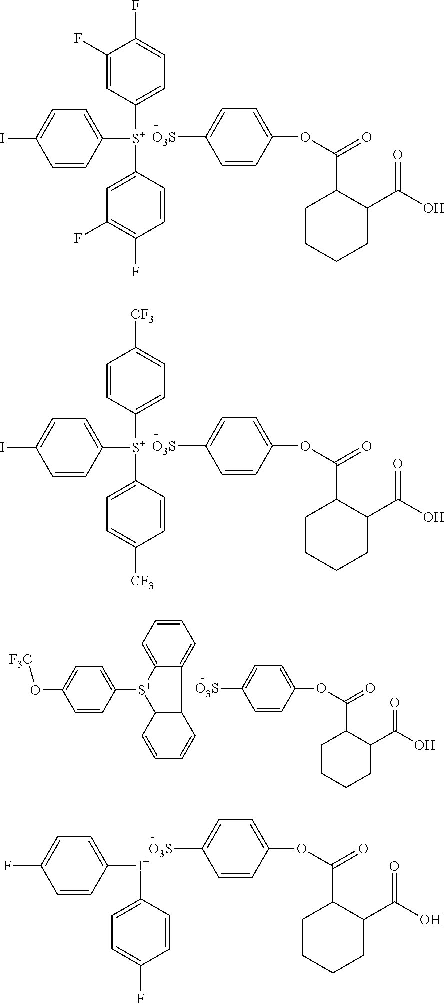

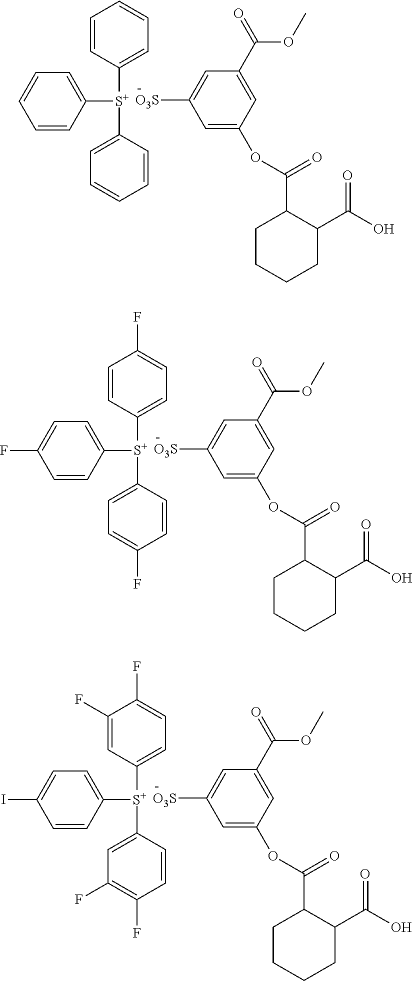

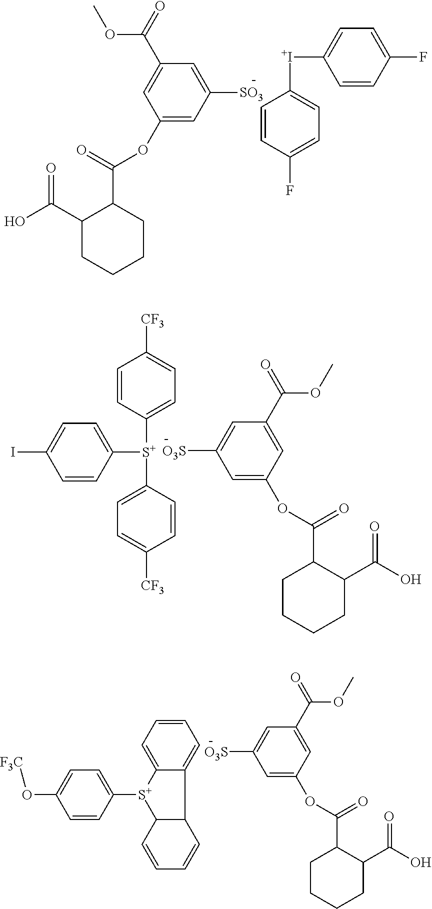

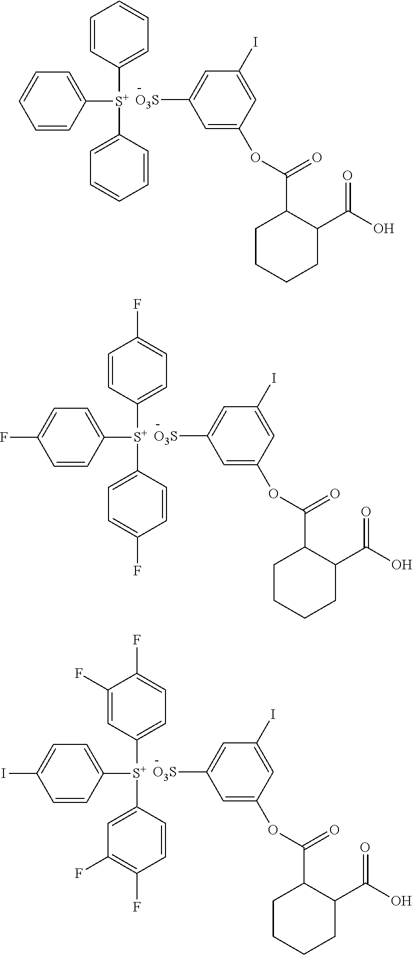

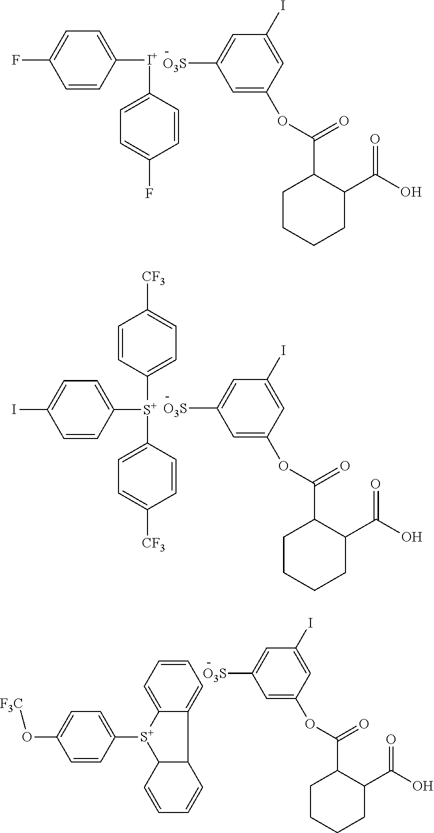

Examples of the compound represented by the formula (I-b1) include the following compounds which are easily available on the market or can also be easily produced by a known production method.

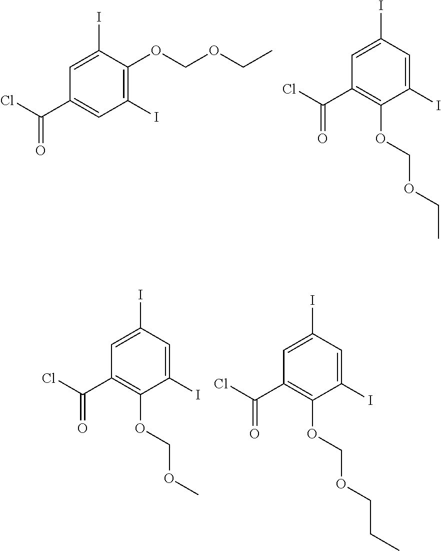

Examples of the compound represented by the formula (I-b2) include the following compounds which are easily available on the market or can also be easily produced by a known production method.

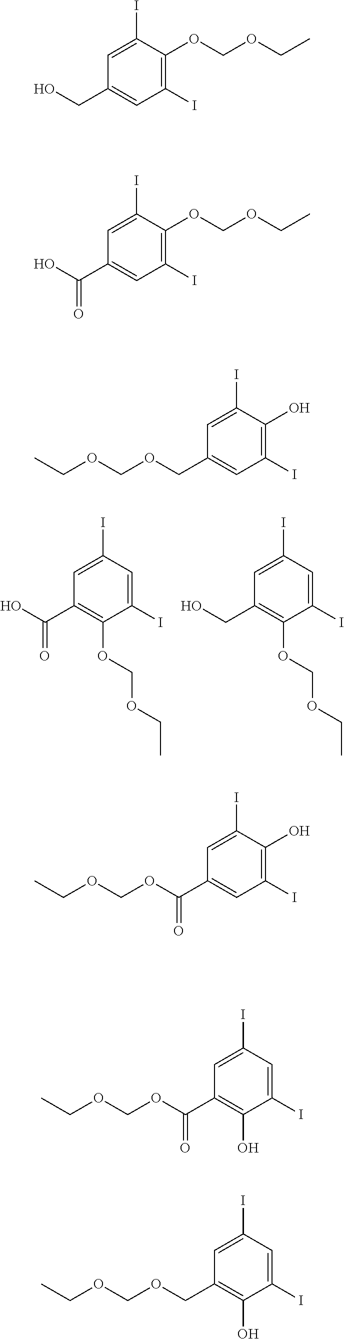

[Structural Unit Derived from Salt Represented by Formula (I)]

The structural unit derived from the salt represented by formula (I) of the present invention is a structural unit represented by the following formula (IP) (hereinafter sometimes referred to as “structural unit (IP)”).

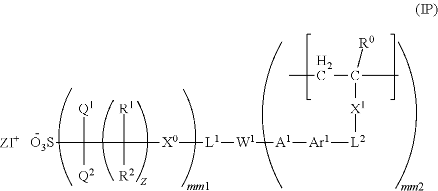

[wherein, in the formula (IP), all symbols have the same meaning as above.]

The structural unit (IP) shows the state where the double bond of CH₂═C—R⁰ contained in salt (I) is cleaved.

Such a structural unit (IP) functions as an acid generating agent similarly to the salt (I) and also functions as a structural unit constituting a compound or resin.

[Resin Containing Structural Unit (IP) Derived from Salt Represented by Formula (I)]

The resin of the present invention is a resin containing a structural unit (IP) (hereinafter sometimes referred to as “resin (Ap)”).

The resin (Ap) may be a homopolymer containing one structural unit (IP) or a copolymer containing two or more structural units (IP).

In addition, the resin (Ap) may contain a structural unit other than the structural unit (IP). Examples of the structural unit other than the structural unit (IP) include structural units having an acid labile group (hereinafter sometimes referred to as “structural unit (a1)”) and structural units other than structural unit (a1), as described below. Examples of the structural units other than the structural unit (a1) include a structural unit not having an acid labile group (hereinafter may be referred to as “structural unit (s)”) and other structural units known in the field. Here, the term “acid labile group” refers to a group that has a leaving group, and in which the leaving group is detached due to contact with an acid, and the structural unit is converted into a structural unit having a hydrophilic group (for example, a hydroxyl group or a carboxy group).

In the resin (Ap), the content ratio of the structural unit (IP) is, for example, 0.1 mol % or more, preferably 0.5 mol % or more, more preferably 0.8 mol % or more, and still more preferably 1 mol % or more, relative to the total structural units of the resin (Ap). Also, it can be 100 mol % or less, preferably 50 mol % or less, more preferably 30 mol % or less, and still more preferably 20 mol % or less. Specifically, it is 0.1 to 100 mol %, preferably 0.5 to 50 mol %, more preferably 0.8 to 30 mol %, and still more preferably 1 to 20 mol %.

In particular, when the resin (Ap) is used in a resist composition as will be described later, it preferably contains the structural unit (a1) in addition to the structural unit (IP).

In addition, when the resin (Ap) is used in a resist composition as will be described later, it may be used in combination with a resin containing the structural unit (a1) (hereinafter sometimes referred to as “resin (A)”) and/or resins other than the resin (A), regardless of whether it contains the structural unit (a1) or not. Hereinafter, the resin (Ap) and/or the resin (A) may be referred to as “resin (A) etc.”

It is preferable that the resin (Ap) and the resin (A) each further contain a structural unit other than the structural unit (a1).

<Structural Unit (a1)>

The structural unit (a1) is derived from a monomer having an acid labile group (hereinafter, may be referred to as “monomer (a1)”).

The acid labile group included in the resin (A) etc. is preferably a group represented by formula (1) (hereinafter, also referred to as group (1)) and/or a group represented by formula (2) (hereinafter, also referred to as group (2)).

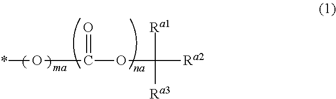

wherein, in the formula (1), R^{a1}, R^{a2}, and R^{a3} each independently represent an alkyl group having 1 to 8 carbon atoms, an alkenyl group having 2 to 8 carbon atoms, an alicyclic hydrocarbon group having 3 to 20 carbon atoms, an aromatic hydrocarbon group having 6 to 18 carbon atoms, or a combined group of these groups, or R^{a1} and R^{a2} are bonded to each other to form an alicyclic hydrocarbon group having 3 to 20 carbon atoms together with the carbon atom to which they are bonded, and the alkyl group, the alkenyl group, the alicyclic hydrocarbon group, the aromatic hydrocarbon group, and the combined group of these groups optionally have a halogen atom;
ma and na each independently represent 0 or 1, and at least one of ma and na represents 1; and
* represents a bonding site.

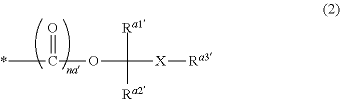

wherein, in the formula (2), R^{a1′} and R^{a2′} each independently represent a hydrogen atom or a hydrocarbon group having 1 to 12 carbon atoms, R^{a3′} represents a hydrocarbon group having 1 to 20 carbon atoms, or R^{a2′} and R^{a3′} are bonded to each other to form a heterocyclic group having 3 to 20 carbon atoms together with the carbon atom to which they are bonded and X, and —CH₂— contained in the hydrocarbon group and the heterocyclic group is optionally replaced with —O— or —S—, and the hydrocarbon group and the heterocyclic group optionally have a halogen atom;
X represents an oxygen atom or a sulfur atom;
na′ represents 0 or 1; and
* represents a bonding site.

Examples of the alkyl groups for R^{a1}, R^{a2}, and R^{a3} include a methyl group, an ethyl group, a propyl group, a butyl group, a pentyl group, a hexyl group, a heptyl group, and an octyl group.

Examples of the alkenyl groups for R^{a1}, R^{a2}, and R^{a3} include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group, and a nonenyl group.

The alicyclic hydrocarbon groups for R^{a1}, R^{a2}, and R^{a3} may be either monocyclic or polycyclic. Examples of the monocyclic alicyclic hydrocarbon group include cycloalkyl groups such as a cyclopentyl group, a cyclohexyl group, a cycloheptyl group, and a cyclooctyl group. Examples of the polycyclic alicyclic hydrocarbon group include a decahydronaphthyl group, an adamantly group, a norbornyl group, and the following groups (* represents a bonding site). The number of carbon atoms in the alicyclic hydrocarbon groups in R^{a1}, R^{a2}, and R^{a3} is preferably 3 to 16.

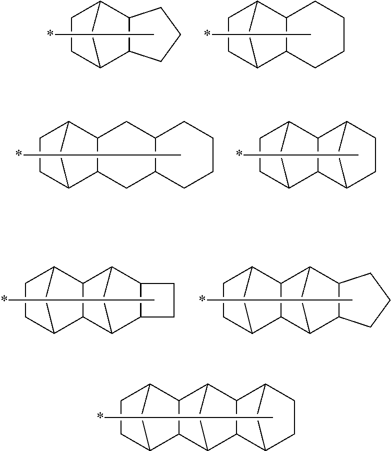

Examples of the aromatic hydrocarbon groups for R^{a1}, R^{a2}, and R³ include aryl groups such as a phenyl group, a naphthyl group, an anthryl group, a biphenyl group, and a phenanthryl group.

Examples of the combined groups include: the above-described combined group of an alkyl group and an alicyclic hydrocarbon group (for example, alkylcycloalkyl groups or cycloalkylalkyl groups such as a methylcyclohexyl group, a dimethylcyclohexyl group, a methylnorbornyl group, a cyclohexylmethyl group, an adamantylmethyl group, an adamantyldimethyl group and a norbornylethyl group); an aralkyl group such as a benzyl group; an aromatic hydrocarbon group having an alkyl group (such as a p-methylphenyl group, a p-tert-butylphenyl group, a tolyl group, a xylyl group, a cumenyl group, a mesityl group, a 2,6-diethylphenyl group, or a 2-methyl-6-ethylphenyl group); an aromatic hydrocarbon group having an alicyclic hydrocarbon group (such as a p-cyclohexylphenyl group and a p-adamantylphenyl group); and an aryl-cycloalkyl group such as a phenylcyclohexyl group.

Preferably, ma is 0 and na is 1.

When R^{a1} and R^{a2} are bonded together to form an alicyclic hydrocarbon group, examples of —C(R^{a1})(R^{a2})(R^{a3}) include the following groups. The alicyclic hydrocarbon group preferably has 3 to 12 carbon atoms. * represents the bonding site to —O—.

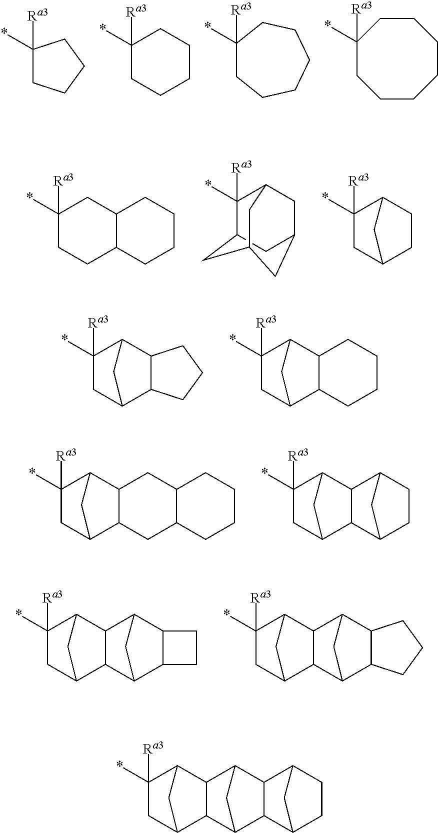

Examples of the hydrocarbon groups for R^{a1′}, R^{a2′}, and R^{a3′} include an alkyl group, an alicyclic hydrocarbon group, an aromatic hydrocarbon group, and the group formed by combining these groups.

Examples of the alkyl group, the alicyclic hydrocarbon group, the aromatic hydrocarbon group, and the group formed by combining these groups include the same groups as those listed for R^{a1}, R^{a2}, and R^{a3}.

When R^{a2′} and R^{a3′} are bonded to each other to form a heterocyclic group together with the carbon atom to which they are bonded and X, examples of —C(R^{a1′})(R^{a2′})—X—R^{a3′} include the following groups. * represents a bonding site.

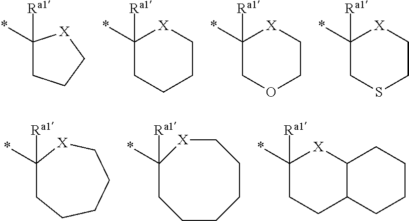

At least one of R^{a1′} and R^{a2′} is preferably a hydrogen atom.

na′ is preferably 0.

The halogen atom that R^{a1}, R^{a2}, R^{a3}, R^{a1′}, R^{a2′}, and R^{a3′} optionally have includes a fluorine atom, a chlorine atom, a bromine atom, or an iodine atom.

Examples of the group (1) include the following groups.

A group in which R^{a1}, R^{a2}, and R^{a3} are alkyl groups, ma=0, and na=1 in formula (1). As the group, a tert-butoxycarbonyl group is preferable.

A group in which R^{a1} and R^{a2} are combined together with the carbon atom to which they are bonded to form an adamantyl group, R^{a3} is an alkyl group, and ma=0 and na=1 in formula (1).

A group in which, R^{a1} and R^{a2} are each independently an alkyl group, Ras is an adamantyl group, and ma=0 and na=1 in formula (1).

Specific examples of the group (1) include the following groups. * represents a bonding site.

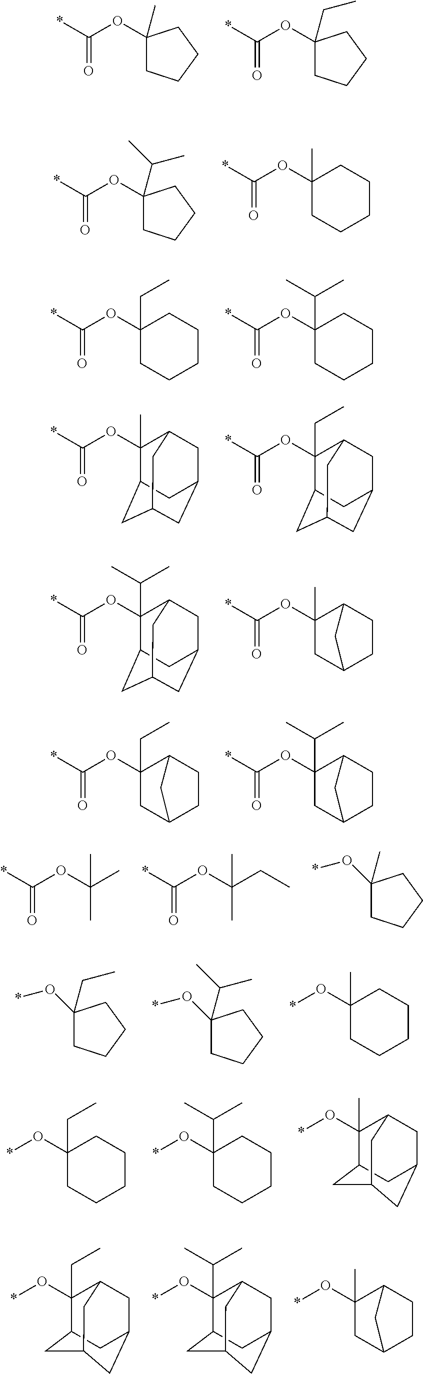

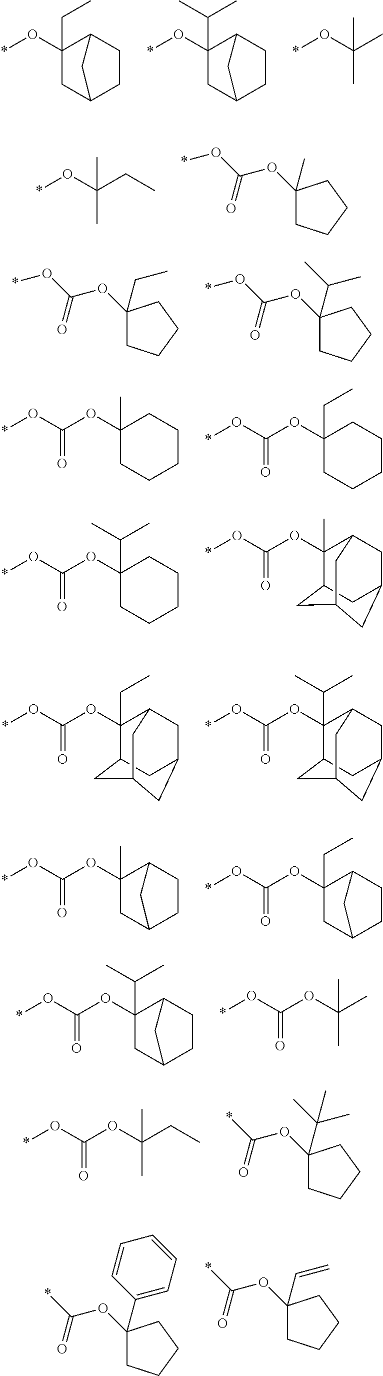

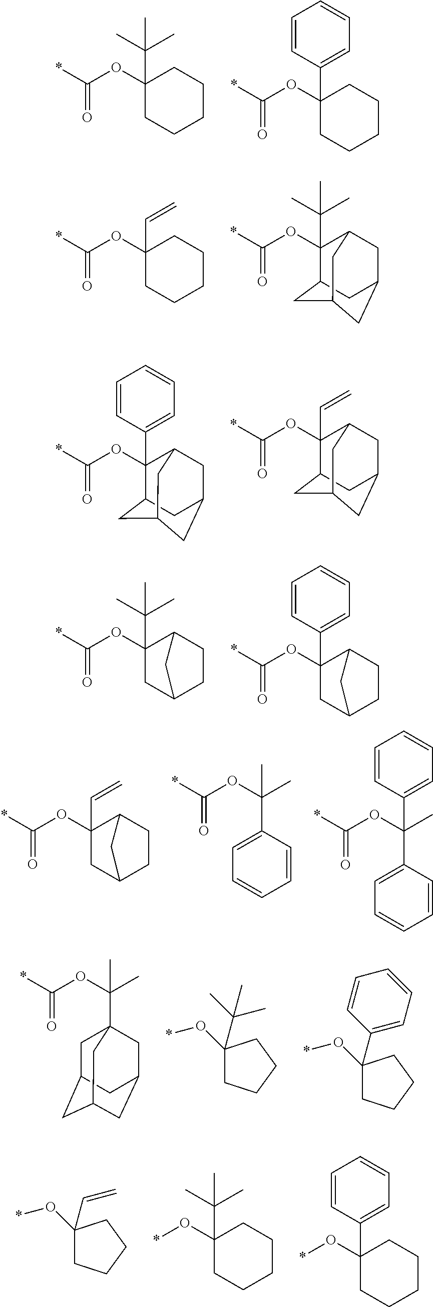

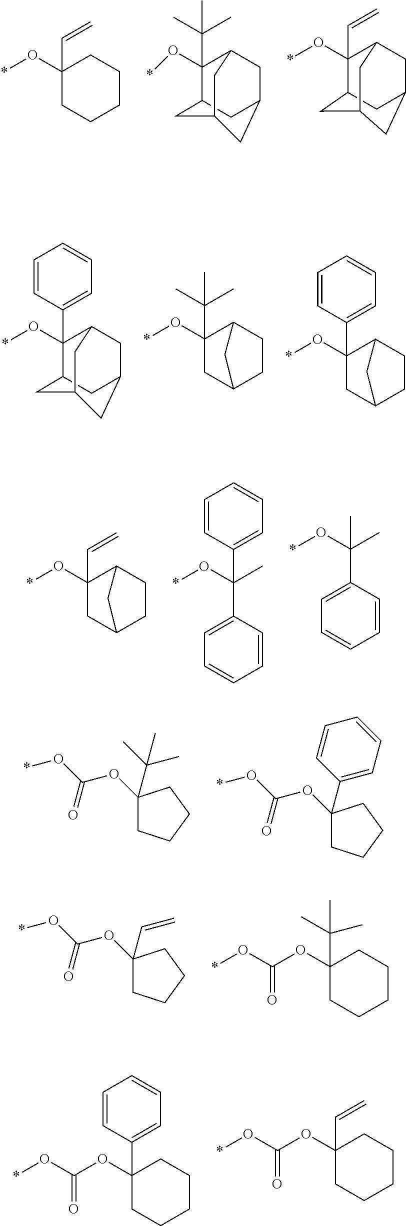

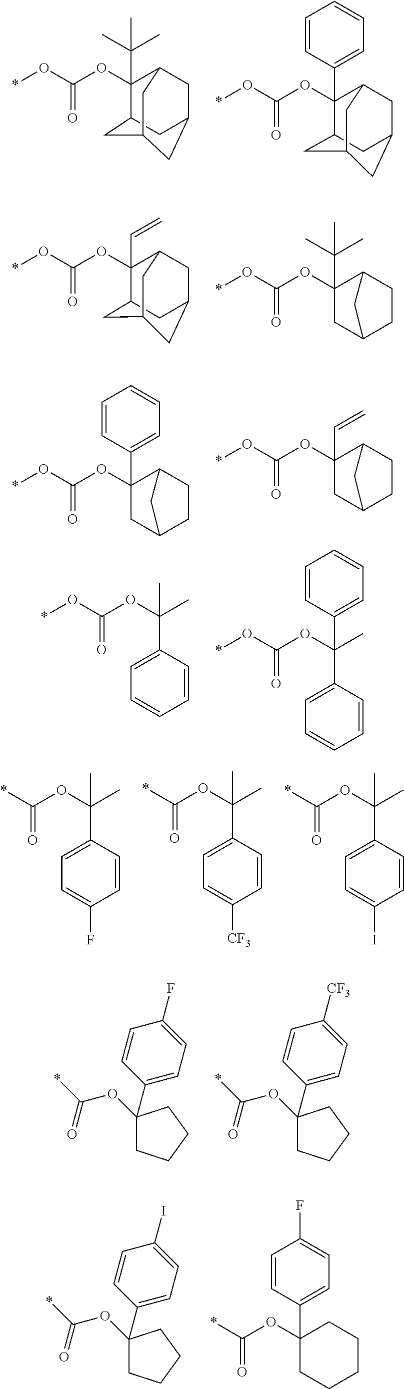

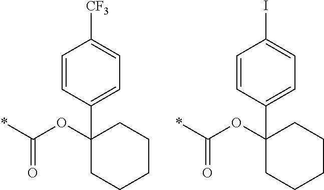

Specific examples of the group (2) include the following groups. * represents a bonding site.

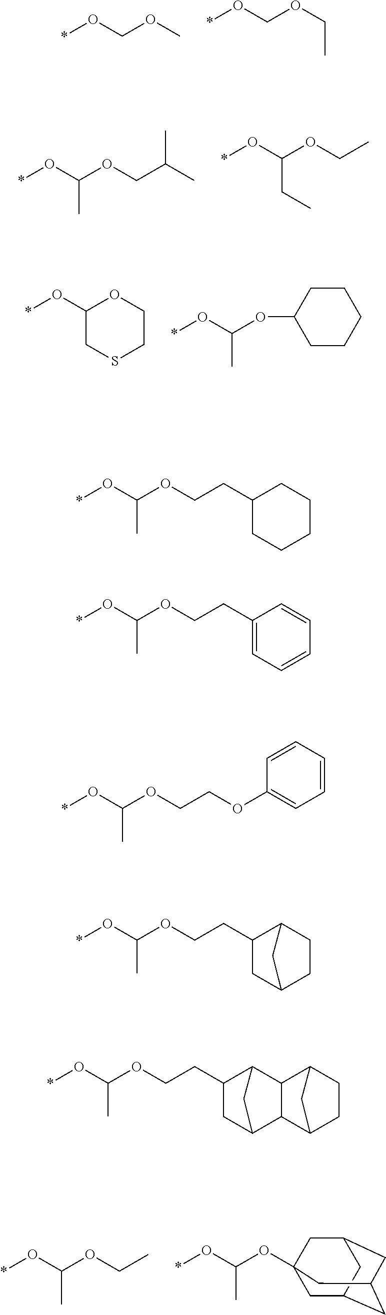

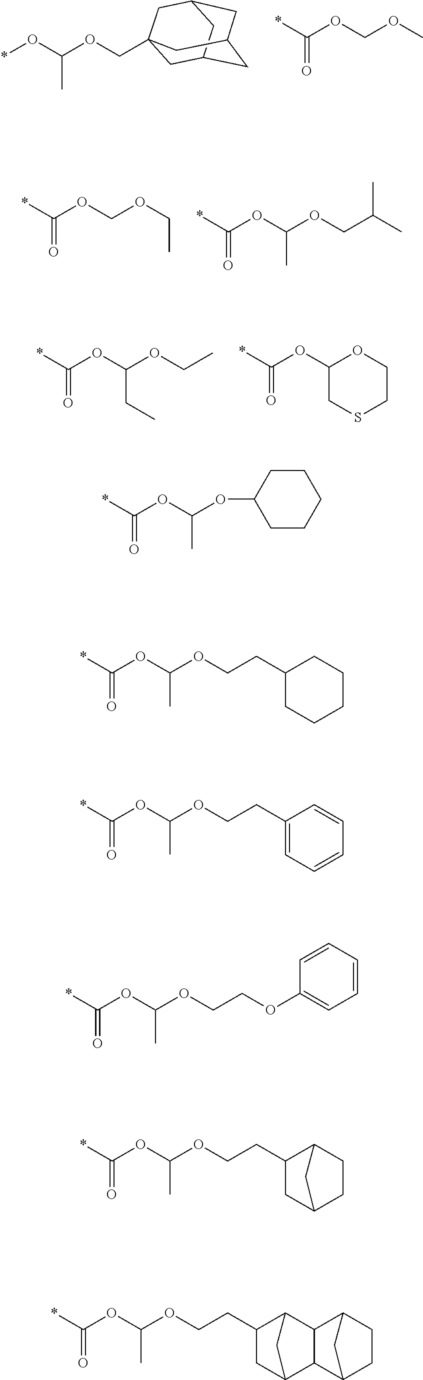

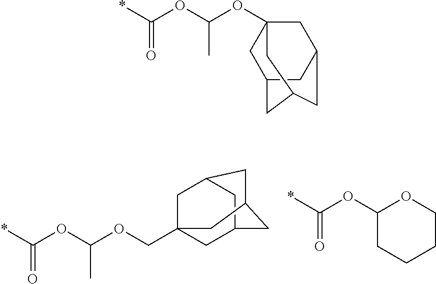

Monomer (a1) is preferably a monomer having an acid labile group and an ethylenically unsaturated bond, more preferably a (meth)acrylic monomer having an acid labile group.

Among the (meth)acrylic monomers having an acid labile group, preferable are those having an alicyclic hydrocarbon group having 5 to 20 carbon atoms. When the resin (A) etc. having a structural unit derived from the monomer (a1) having a bulky structure such as an alicyclic hydrocarbon group is used in a resist composition, the resolution of the resist pattern can be improved.

Examples of the structural unit derived from a (meth)acrylic monomer having the group (1) include a structural unit represented by formula (a1-0) (hereinafter, may be referred to as structural unit (a1-0)), a structural unit represented by formula (a1-1) (hereinafter, may be referred to as structural unit (a1-1)), or a structural unit represented by formula (a1-2) (hereinafter, may be referred to as structural unit (a1-2)). Preferably, it is at least one structural unit selected from the group consisting of the structural unit (a1-0), structural unit (a1-1), and structural unit (a1-2), and more preferably, it is at least one or two structural units selected from the group consisting of the structural unit (a1-1) and structural unit (a1-2). These may be used singly or in combination of two or more.

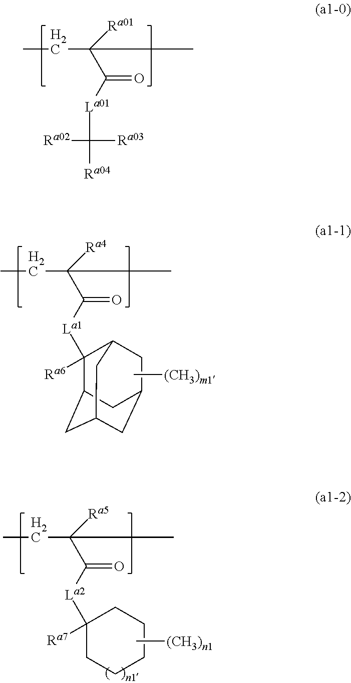

wherein, in the formula (a1-0), the formula (a1-1), and the formula (a1-2), all symbols have the same meaning as defined above.

Examples of the halogen atoms in R^{a01}, R^{a4}, and R^{a5} include a fluorine atom, a chlorine atom, a bromine atom, and an iodine atom.

Examples of the alkyl group optionally having a halogen atom in R^{a01}, R^{a4}, and R^{a5} include a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a chloromethyl group, a bromomethyl group, and an iodomethyl group. The number of carbon atoms in the alkyl group is preferably 1 to 4, more preferably 1 to 3, and further preferably 1 or 2. The alkyl group is preferably a methyl group or an ethyl group, and more preferably a methyl group.

R^{a01}, R^{a4}, and R^{a5} are preferably a hydrogen atom or a methyl group, and more preferably a methyl group.

L^{a01}, L^{a1}, and L^{a2} are preferably an oxygen atom or *—O—(CH₂)ₖ₀₁—CO—O— (wherein k01 is preferably an integer of 1 to 4, and more preferably 1), and are more preferably an oxygen atom.

Examples of the alkyl group, the alkenyl group, the alicyclic hydrocarbon group, the aromatic hydrocarbon group, and the combined group of these groups for R^{a02}, R^{a03}, R^{a04}, R^{a06}, and R^{a07} include the same groups as those listed for R^{a1}, R^{a2}, and R^{a3} in the formula (1).

The alkyl group in R^{a02}, R^{a03}, and R^{a04} is preferably an alkyl group having 1 to 6 carbon atoms, more preferably a methyl group or an ethyl group, and further preferably a methyl group.

The alkyl group in R^{a6} and R^{a7} is preferably an alkyl group having 1 to 6 carbon atoms, more preferably a methyl group, an ethyl group, an isopropyl group, or a t-butyl group, and further preferably an ethyl group, an isopropyl group, or a t-butyl group.

The alkenyl group in R^{a6} and R^{a7} is preferably an alkenyl group having 2 to 6 carbon atoms, more preferably an ethenyl group, a propenyl group, an isopropenyl group, or a butenyl group.

The number of carbon atoms in the alicyclic hydrocarbon group in R^{a02}, R^{a03}, R^{a04}, R^{a6}, and R^{a7} is preferably 5 to 12, and more preferably 5 to 10.

The number of carbon atoms in the aromatic hydrocarbon group of R^{a02}, R^{a03}, R^{a04}, R^{a6}, and R^{a7} is preferably 6 to 12, and more preferably 6 to 10.

In the case of a combined group of an alkyl group and an alicyclic hydrocarbon group, the total number of carbon atoms in the combination of the alkyl group and the alicyclic hydrocarbon group is preferably 18 or less.

In the case of a combined group of an alkyl group and an aromatic hydrocarbon group, the total number of carbon atoms in the combination of the alkyl group and the aromatic hydrocarbon group is preferably 18 or less.

R^{a02} and R^{a03} are preferably an alkyl group having 1 to 6 carbon atoms and optionally having a halogen atom or an aromatic hydrocarbon group having 6 to 12 carbon atoms and optionally having a halogen atom, and more preferably a methyl group, an ethyl group, a phenyl group, or a naphthyl group, optionally having a halogen atom.

R^{a04} is preferably an alkyl group having 1 to 6 carbon atoms and optionally having a halogen atom or an alicyclic hydrocarbon group having 5 to 12 carbon atoms and optionally having a halogen atom, and more preferably a methyl group, an ethyl group, a cyclohexyl group, or an adamantyl group, optionally having a halogen atom.

R^{a6} and R^{a7} are preferably an alkyl group having 1 to 6 carbon atoms and optionally having a halogen atom, an alkenyl group having 2 to 6 carbon atoms and optionally having a halogen atom, or an aromatic hydrocarbon group having 6 to 12 carbon atoms and optionally having a halogen atom, more preferably a methyl group, ethyl group, isopropyl group, t-butyl group, ethenyl group, phenyl group or naphthyl group, optionally having a halogen atom, and even more preferably an ethyl group, isopropyl group, t-butyl group, ethenyl group or phenyl group, optionally having a halogen atom.

m1′ is preferably an integer of 0 to 3, more preferably 0 or 1.

n1 is preferably an integer of 0 to 10, more preferably an integer of 0 to 8, further preferably an integer of 0 to 3, and still more preferably 0 or 1.

n1′ is preferably 0 or 1.

Examples of the structural unit (a1-0) include a structural unit represented by any one of formulas (a1-0-1) to (a1-0-24) and a structural unit in which the methyl group corresponding to R^{a01} in the structural unit (a1-0) is replaced with a hydrogen atom, a halogen atom, and a haloalkyl group (an alkyl group having a halogen atom) or another alkyl group, and a structural unit represented by any one of formulas (a1-0-1) to (a1-0-10), (a1-0-13), (a1-0-14), and (a1-0-19) to (a1-0-24) is preferable.

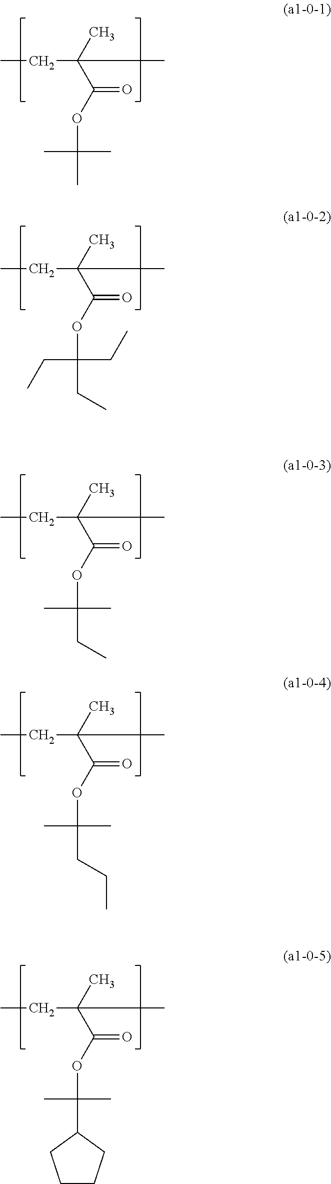

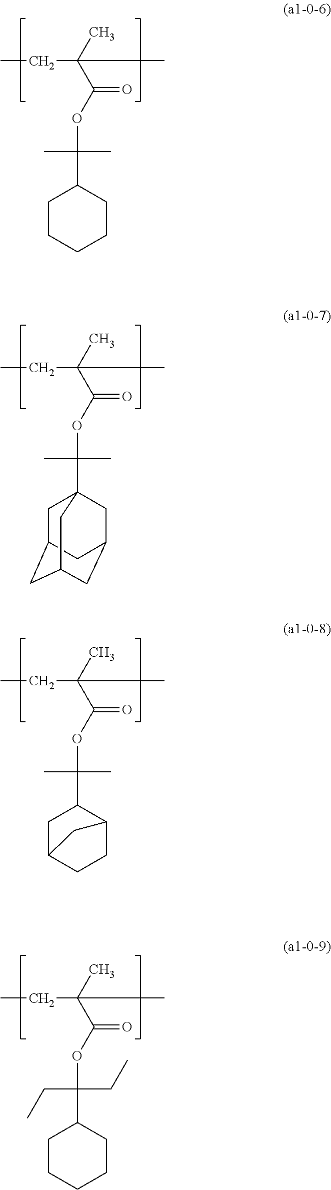

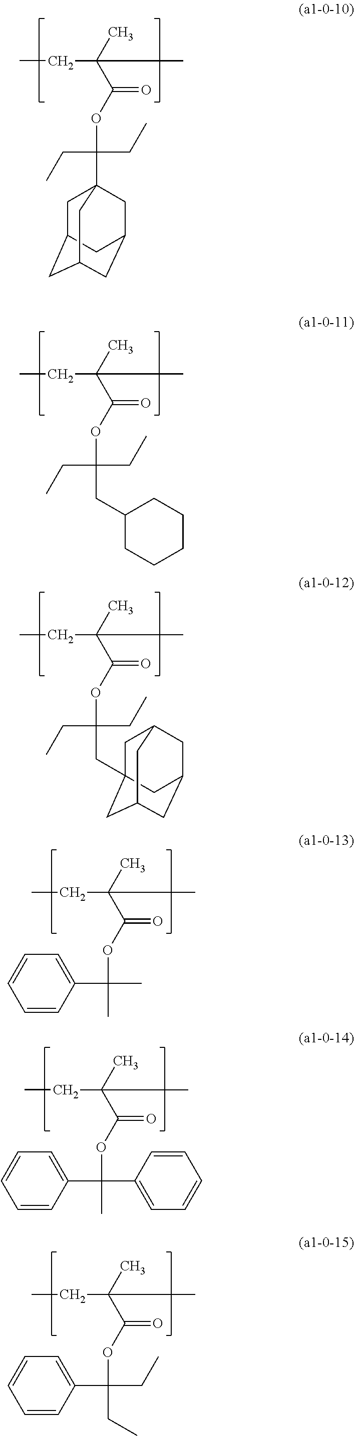

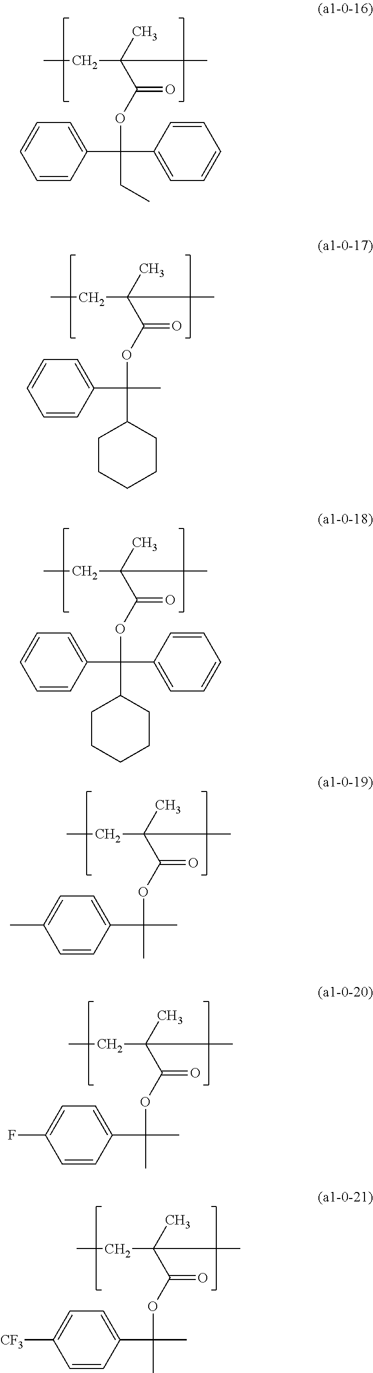

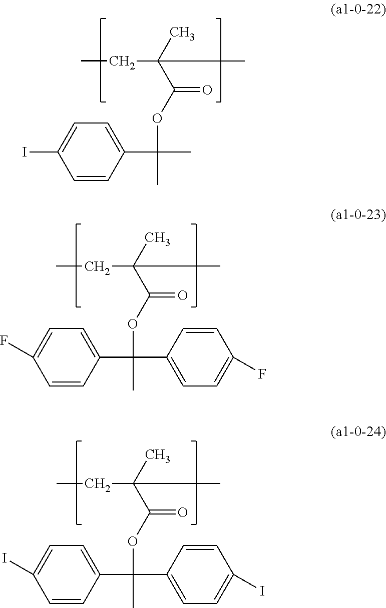

Examples of the structural unit (a1-1) include a structural unit derived from a monomer disclosed in Japanese Patent Laid-open No. 2010-204646. Among these, a structural unit represented by any of formulas (a1-1-1) to (a1-1-7) and a structural unit in which the methyl group corresponding to R^{a4} in the structural unit (a1-1) is replaced with a hydrogen atom, a halogen atom, a haloalkyl group, or another alkyl group are preferable, and a structural unit represented by any of formulas (a1-1-1) to (a1-1-4) is more preferable.

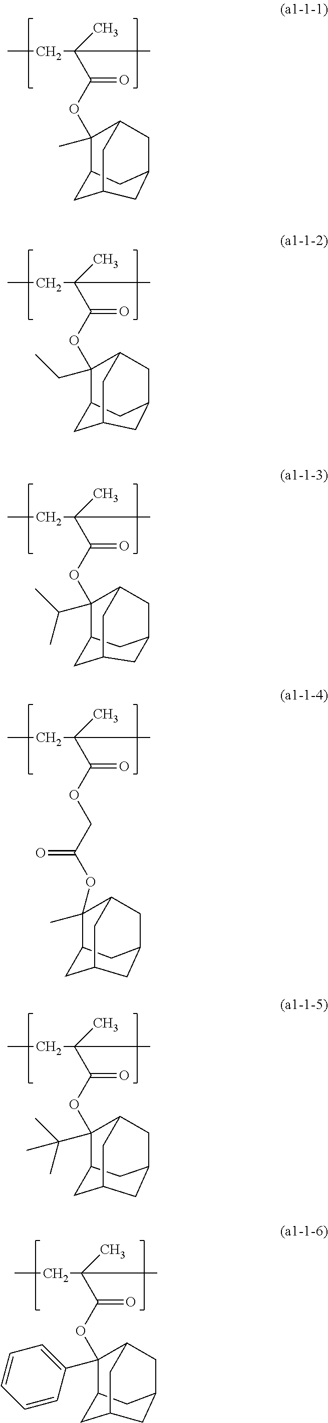

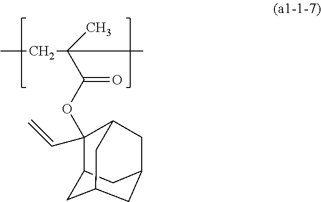

Examples of the structural unit (a1-2) include a structural unit represented by any one of formulas (a1-2-1) to (a1-2-20) or a structural unit in which the methyl group corresponding to R^{a5} in the structural unit (a1-2) is replaced with a hydrogen atom, a halogen atom, a haloalkyl group, or another alkyl group, and a structural unit represented by any one of formulas (a1-2-2), (a1-2-5), (a1-2-6), and (a1-2-10) to (a1-2-20) is preferable.

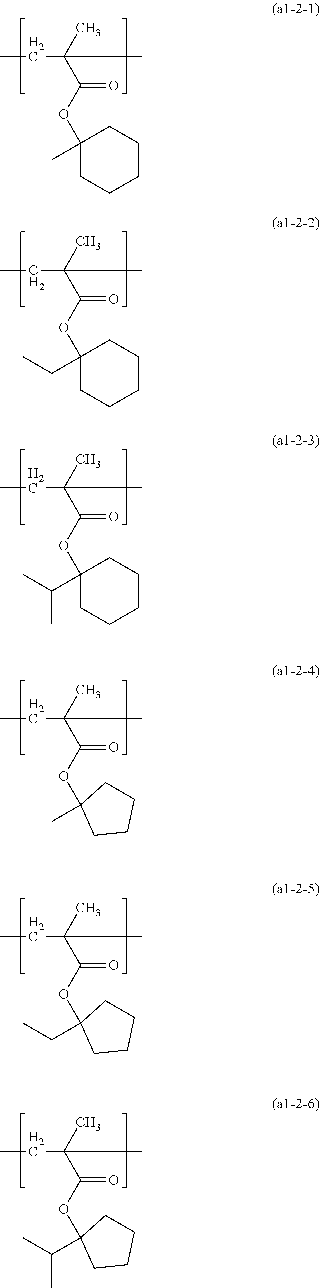

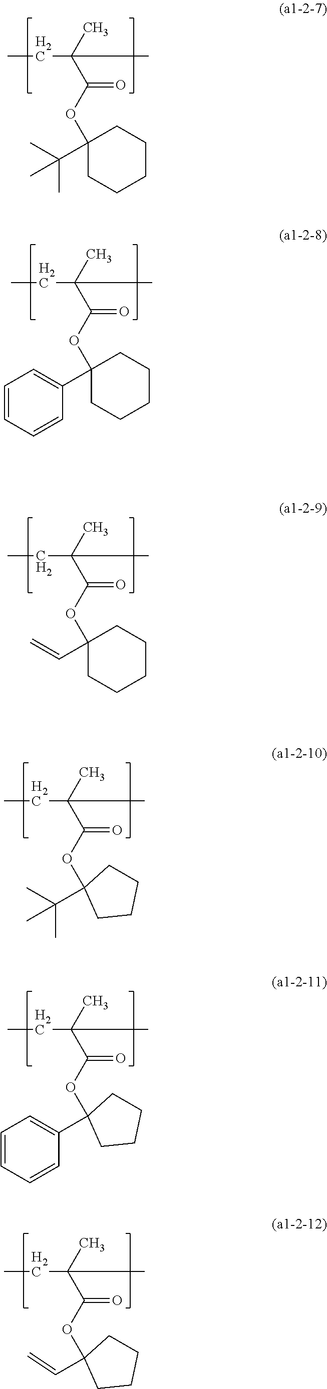

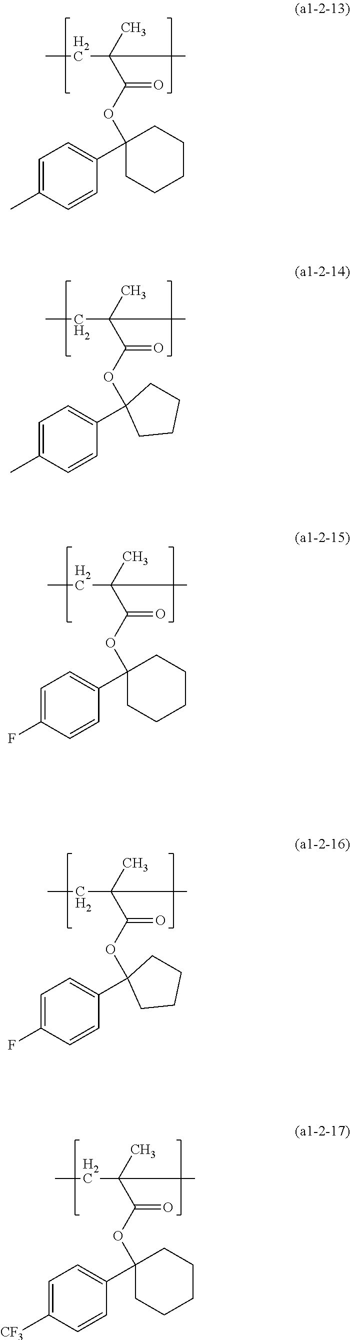

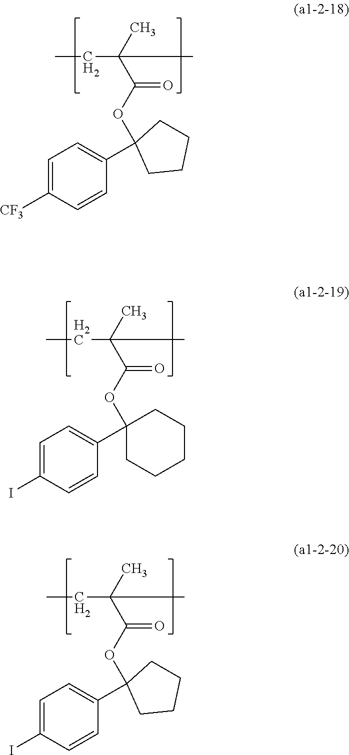

When the resin (A) etc. includes the structural unit (a1-0) and/or the structural unit (a1-1) and/or the structural unit (a1-2), the total content ratio of these, with respect to all structural units of the resin (A) etc., is, for example, 10 mol % or more, preferably 15 mol % or more, more preferably 20 mol % or more, further preferably 25 mol % or more, still more preferably 30 mol % or more, further more preferably 40 mol % or more, and most preferably 50 mol % or more. In addition, the content may be 95 mol % or less, preferably 90 mol % or less, more preferably 85 mol % or less, further preferably 70 mol % or less, and still more preferably 65 mol % or less. Specifically, 10 to 95 mol % is exemplified, preferably 15 to 90 mol %, more preferably 20 to 85 mol %, further preferably 25 to 70 mol %, and still more preferably 30 to 70 mol %.

When the resin (A) etc. includes the structural unit (a1-0), the content ratio thereof, with respect to all structural units of the resin (A) etc., 5 mol % or more is exemplified, preferably 10 mol % or more, more preferably 15 mol % or more, further preferably 20 mol % or more, still more preferably 25 mol % or more, even more preferably 30 mol % or more, and most preferably 35 mol % or more. In addition, the content may be 80 mol % or less, preferably 75 mol % or less, more preferably 70 mol % or less, further preferably 65 mol % or less, and still more preferably 60 mol % or less. Specifically, 5 to 80 mol % is exemplified, preferably 5 to 75 mol %, more preferably 10 to 70 mol %, further preferably 10 to 65 mol %, and still more preferably 10 to 60 mol %.

When the resin (A) etc. includes the structural unit (a1-1) and/or structural unit (a1-2), the total content ratio of these, with respect to all structural units of the resin (A) etc., 10 mol % or more is exemplified, preferably 15 mol % or more, more preferably 20 mol % or more, further preferably 25 mol % or more, still more preferably 30 mol % or more, even more preferably 40 mol % or more, and most preferably 50 mol % or more. In addition, 95 mol % or less is exemplified, preferably 90 mol % or less, more preferably 85 mol % or less, further preferably 80 mol % or less, still more preferably 75 mol % or less, even more preferably 70 mol % or less, and most preferably 65 mol % or less. Specifically, 10 to 90 mol % is exemplified, preferably 15 to 85 mol %, more preferably 15 to 80 mol %, further preferably 20 to 80 mol %, still more preferably 20 to 75 mol %, even more preferably 20 to 70 mol %, even still more preferably 20 to 60 mol %, and most preferably 20 to 55 mol %.

An example of the structural unit (a1) having group (2) includes the structural unit represented by formula (a1-4) (hereinafter, may be referred to as “structural unit (a1-4)”).

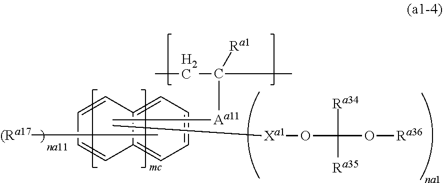

wherein, in the formula (a1-4), all symbols have the same meaning as above.

Examples of the halogen atoms and alkyl groups optionally having halogen atoms in R^{a1} and R^{a17} include the same as those exemplified for R^{a01}, R^{a4}, and R^{a5} in formulas (a1-0) to (a1-2).

R^{a1} is preferably a hydrogen atom or an alkyl group having 1 to 4 carbon atoms, more preferably a hydrogen atom, a methyl group or an ethyl group, and still more preferably a hydrogen atom or a methyl group.

The alkoxy group, alkoxyalkyl group, alkoxyalkoxy group, alkylcarbonyl group, and alkylcarbonyloxy group in R^{a17} include the same as those exemplified in this specification, within the range permitted by the upper limit of the number of carbon atoms. The number of carbon atoms of the alkoxy group is preferably 1 to 4, and is more preferably 1 to 3. The alkoxy group is preferably an alkoxy group having 1 to 4 carbon atoms, more preferably an alkoxy group having 1 to 3 carbon atoms, and further preferably a methoxy group or an ethoxy group, and still more preferably a methoxy group. The number of carbon atoms of the alkoxyalkyl group is preferably 2 to 8, and is more preferably 2 to 4. The alkoxyalkyl group is preferably an alkoxyalkyl group having 2 to 8 carbon atoms, more preferably an alkoxyalkyl group having 2 to 4 carbon atoms, and further preferably a methoxymethyl group or an ethoxyethyl group, and still more preferably a methoxymethyl group. The number of carbon atoms of the alkoxyalkoxy group is preferably 2 to 8, and is more preferably 2 to 4. The alkoxyalkoxy group is preferably an alkoxyalkoxy group having 2 to 8 carbon atoms, more preferably an alkoxyalkoxy group having 2 to 4 carbon atoms, and further preferably a methoxyethoxy group or an ethoxyethoxy group. The alkylcarbonyl group is preferably an alkylcarbonyl group having 2 to 4 carbon atoms, more preferably an alkylcarbonyl group having 2 to 3 carbon atoms, and further more preferably an acetyl group. The alkylcarbonyloxy group is preferably an alkylcarbonyloxy group having 2 to 4 carbon atoms, more preferably an alkylcarbonyloxy group having 2 to 3 carbon atoms, and further more preferably an acetyloxy group.

R^{a17} is preferably a halogen atom, a hydroxy group, a carboxy group, an alkyl group having 1 to 4 carbon atoms optionally having a halogen atom, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and still more preferably a fluorine atom, an iodine atom, a hydroxy group, a methyl group, a methoxy group, or an ethoxyethoxy group.

Examples of the alkanediyl group of A^{a11} include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and dodecane-1,12-diyl group; a branched alkanediyl group such as a butane-1,3-diyl group, a 2-methylpropane-1,3-diyl group, a 2-methylpropane-1,2-diyl group, a pentane-1,4-diyl group, a 2-methylbutane-1,4-diyl group, a heptane-1,6-diyl group, an octane-1,7-diyl group, a nonane-1,8-diyl group, a decane-1,9-diyl group, and an undecane-1,10-diyl group. The number of carbon atoms in the alkanediyl group is preferably 1 to 10, more preferably 1 to 8, further preferably 1 to 6, still more preferably 1 to 4, even more preferably 1 to 3, and even still more preferably 1 or 2.

Examples of the alkyl group of R^{a18} include a methyl group, an ethyl group, an n-propyl group, an isopropyl group, an n-butyl group, and a tert-butyl group.

When —CH₂— contained in the alkanediyl group of A^{a11} is replaced with —O—, —CO—, or —NR^{a18}—, the number of carbon atoms before the replacement is regarded as the number of carbon atoms of the alkanediyl group.

Examples of the group formed by replacing —CH₂— in the alkanediyl group of A^{a11} with —O—, —CO—, or —NR^{a18}— include a hydroxy group, a carboxy group, a carbonyl group, an oxy group, an amino group, an alkoxy group, an alkoxycarbonyl group, an alkylcarbonyl group, an alkylamino group, a peptide group, an alkanediyloxy group, an alkanediyloxycarbonyl group, an alkanediylcarbonyl group, an alkanediylcarbonyloxy group, an alkanediylsulfonyl group, an alkanediylthio group, and an alkanediylamino group. Examples of these replaced groups include the same ones as those exemplified in this specification, within the range permitted by the upper limit of the number of carbon atoms.

For example, examples of the groups formed by replacing —CH₂— in the alkanediyl group of A^{a11} with —O—, —CO—, or —NR^{a18}— include *—O—, *—CO—O—, *—O—CO—, *—CO—O-A^{a12}-CO—O—, *—O—CO-A^{a12}-O—, *—O-A^{a12}-CO—O—, *—CO—O-A^{a12}-O—CO—, *—O—CO-A^{a12}-O—CO—, and *—CO—NR^{a18}—. Among these, *—CO—O—, *—CO—O-A^{a12}-CO—O—, *—O-A^{a12}-CO—O—, and *—CO—NR^{a18}— are preferable. Herein, A^{a12} represents an alkanediyl group having 1 to 8 carbon atoms, and * represents the bonding site with the carbon atom to which R^{a1} is bonded. Examples of the alkanediyl group of A^{a12} include the same alkanediyl group as A^{a11}, within the range permitted by the upper limit of the number of carbon atoms.

A^{a11} is preferably a single bond, *—CO—O—, or *—CO—O-A^{a12}-CO—O—, more preferably a single bond, *—CO—O— or *—CO—O—CH₂—CO—O—, and still more preferably a single bond or *—CO—O—.
na1 is preferably 1, 2, 3, or 4, more preferably 1, 2, or 3, and further preferably 1 or 2.
na11 is preferably 0, 1, 2, or 3, more preferably 0, 1, or 2, and further preferably 0 or 1.
mc is preferably 0 or 1.

Examples of R^{a34}, R^{a35}, and R^{a36} include those similar for R^{a1′}, R^{2′}, and R^{3′} in formula (2).

Examples of the divalent heterocyclic group having 3 to 20 carbon atoms formed by bonding R^{a35} and R^{a36} together with —C—O— to which they are bonded include the following groups. * represents a bonding site, and one side is a bonding site to R^{a34}.

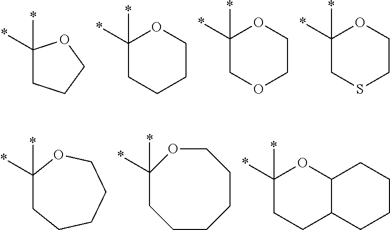

R^{a34} is preferably a hydrogen atom.

R^{a35} is preferably a hydrogen atom, an alkyl group having 1 to 12 carbon atoms, or an alicyclic hydrocarbon group having 3 to 12 carbon atoms, more preferably a methyl group or an ethyl group.

The hydrocarbon group of R^{a36} is preferably an alkyl group having 1 to 18 carbon atoms, an alicyclic hydrocarbon group having 3 to 18 carbon atoms, an aromatic hydrocarbon group having 6 to 18 carbon atoms, or a group formed by combining these, and more preferably an alkyl group having 1 to 18 carbon atoms, an alicyclic hydrocarbon group having 3 to 18 carbon atoms, or an aralkyl group having 7 to 18 carbon atoms. The alkyl group and alicyclic hydrocarbon group of R^{a36} are preferably unsubstituted. The aromatic hydrocarbon group in R^{a36} is preferably an aromatic ring having an aryloxy group having 6 to 10 carbon atoms.

The —O—C(R^{a34})(R^{a35})—O—R^{a36} in the structural unit (a1-4) is eliminated upon contact with an acid (e.g., p-toluenesulfonic acid) to form a hydroxy group or a carboxy group.

In the case of a benzene ring, —X^{a1}—O—C(R^{a34})(R^{a35})—O—R^{a36} may be bonded to any of the o-position, m-position, or p-position with respect to the bonding position of A^{a11}. Among these, at least one is preferably bonded to the o-position, or p-position, and more preferably bonded to the p-position. In the case of a naphthalene ring, when the bonding position of A^{a11} is the 1st position, the above group may be bonded to any of the 2nd to 8th positions, and when the bonding position of A^{a11} is the 2nd position, the above group may be bonded to any of the 1st and 3rd to 8th positions. Among these, when the bonding position of A^{a11} is the 1st position, at least one is preferably bonded to the 3rd to 6th positions, and more preferably bonded to the 3rd or 4th position. When the bonding position of A^{a11} is the 2nd position, the above group is preferably bonded to the 4th to 7th positions, and more preferably bonded to the 5th or 6th position.

Examples of the structural unit (a1-4) include structural units derived from monomers disclosed in Japanese Patent Laid-Open No. 2010-204646. Preferable are the structural units represented by formulas (a1-4-1) to (a1-4-42) and structural units in which the hydrogen atom corresponding to R^{a1} in the structural unit (a1-4) is replaced with a halogen atom, a haloalkyl group, or an alkyl group, and more preferable are the structural units represented by formulas (a1-4-1) to (a1-4-5), (a1-4-10), (a1-4-13), (a1-4-14), (a1-4-19), and (a1-4-20).

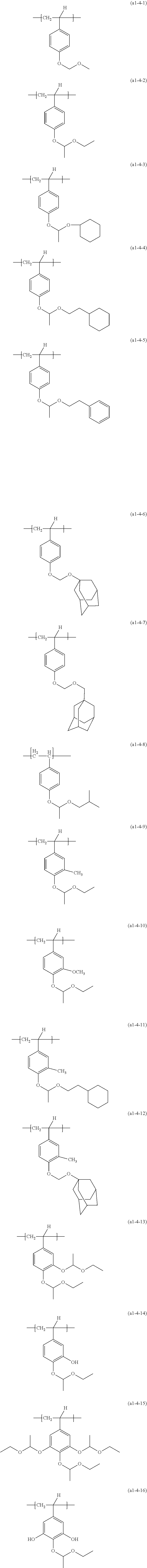

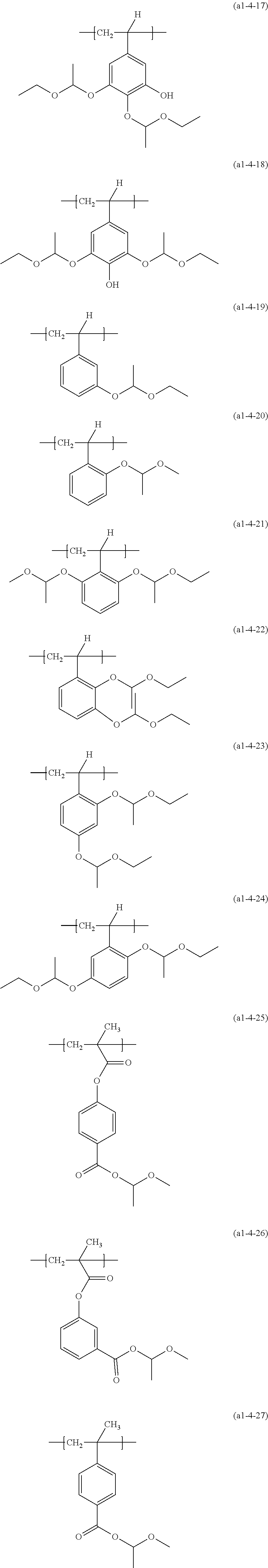

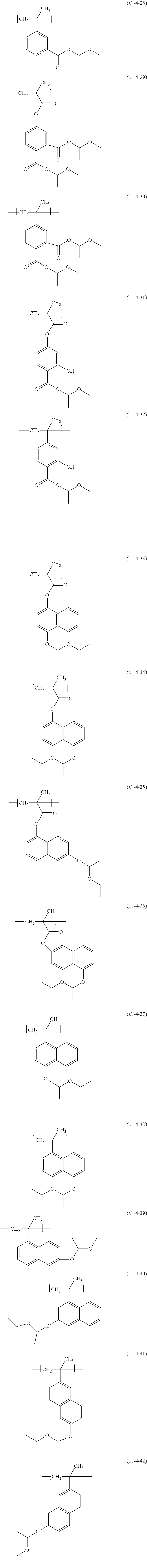

When the resin (A) etc. includes the structural unit (a1-4), the content ratio thereof, with respect to the total of all structural units in the resin (A) etc., 10 mol % or more is exemplified, preferably 15 mol % or more, more preferably 20 mol % or more, further preferably 25 mol % or more, still more preferably 30 mol % or more, even more preferably 40 mol % or more, and most preferably 50 mol % or more. In addition, 95 mol % or less is exemplified, it is preferably 90 mol % or less, more preferably 85 mol % or less, further preferably 80 mol % or less, still more preferably 75 mol % or less, even more preferably 70 mol % or less, and most preferably 65 mol % or less. Specifically, it is preferably 10 to 95 mol %, more preferably 15 to 90 mol %, further preferably 20 to 85 mol %, still more preferably 20 to 70 mol %, and particularly preferably 20 to 60 mol %.

The structural unit derived from the (meth)acrylic monomer having group (2) may be the structural unit represented by formula (a1-5) (hereinafter may be referred to as “structural unit (a1-5)”).

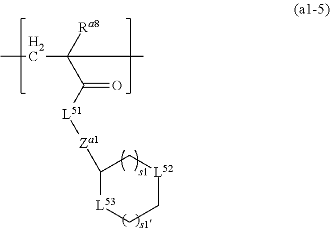

[wherein, in formula (a1-5), all symbols have the same meaning as above.]

Examples of the halogen atoms and alkyl groups optionally having halogen atoms in R^{a8} in formula (a1-5) include the same as those exemplified for R^{a01}, R^{a4}, and R^{a8} in formulas (a1-0) to (a1-2).

In formula (a1-5), R^{a8} is preferably a hydrogen atom, a methyl group, or a trifluoromethyl group.

L⁵¹ is preferably an oxygen atom.

It is preferable that one of L⁵² and L⁵³ is —O— and the other is —S—.

s1 is preferably 1.

s1′ is preferably an integer of 0 to 2.

Z^{a1} is preferably a single bond or *—CH₂—CO—O—.

Examples of the structural unit (a1-5) include structural units derived from monomers disclosed in Japanese Patent Laid-Open No. 2010-61117. Among these, the structural units represented by formulas (a1-5-1) to (a1-5-4) are preferable, and the structural unit represented by formula (a1-5-1) or (a1-5-2) is more preferable.

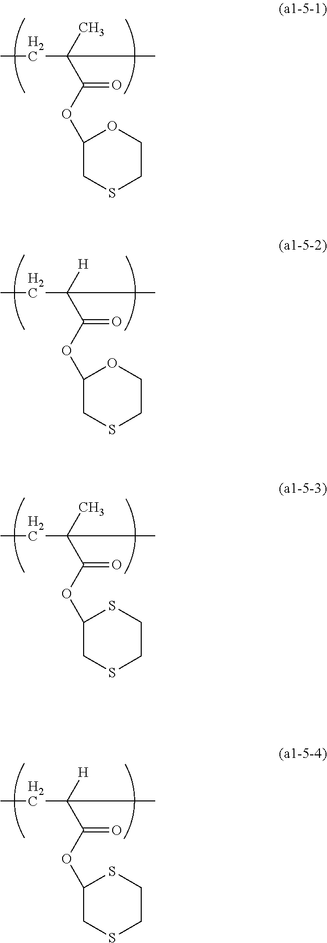

When the resin (A) etc. includes the structural unit (a1-5), the content ratio thereof, with respect to all structural units of the resin (A) etc., 1 mol % or more is exemplified, preferably 2 mol % or more, more preferably 3 mol % or more, further preferably 5 mol % or more, still more preferably 10 mol % or more, even more preferably 20 mol % or more, and most preferably 25 mol % or more. In addition, 80 mol % or less is exemplified, preferably 70 mol % or less, more preferably 60 mol % or less, further preferably 50 mol % or less, still more preferably 45 mol % or less, even more preferably 40 mol % or less, and most preferably 30 mol % or less. Specifically, 1 to 50 mol % is preferable, 3 to 45 mol % is more preferable, 5 to 40 mol % is further preferable, and 5 to 30 mol % is still more preferable.

An example of the structural unit (a1) having group (1) is the structural unit represented by formula (a1-6) (hereinafter, may be referred to as “structural unit (a1-6)”).

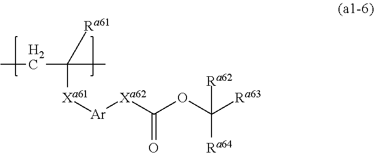

[wherein, in formula (a1-6), all symbols have the same meaning as above.]

Examples of the halogen atoms and alkyl groups optionally having halogen atoms in R^{a61} in formula (a1-6) include the same as those exemplified for R^{a01}, R^{a4}, and R^{a5} in formulas (a1-0) to (a1-2).

R^{a61} is preferably a hydrogen atom, a methyl group, or a trifluoromethyl group.

As the alkyl group in R^{a62}, R^{a63}, R^{a64}, and R^{a65}, a methyl group, an ethyl group, a propyl group, a butyl group, a pentyl group, a hexyl group, and the like are preferable. As the alkyl group, an alkyl group having 1 to 4 carbon atoms is preferable, an alkyl group having 1 to 3 carbon atoms is more preferable, and a methyl group or an ethyl group is further preferable.

Examples of the cyclic hydrocarbon group in R^{a62}, R^{a63}, and R^{a64} include an alicyclic hydrocarbon group and an aromatic hydrocarbon group.

The alicyclic hydrocarbon group may be either monocyclic or polycyclic. Examples of the monocyclic alicyclic hydrocarbon group include a cyclopentyl group, a cyclohexyl group, a cycloheptyl group, and a cyclooctyl group. Examples of the polycyclic alicyclic hydrocarbon group include a decahydronaphthyl group, an adamantyl group, and a norbornyl group. The number of carbon atoms in the alicyclic hydrocarbon group is preferably 3 to 16, and more preferably 3 to 12.

Examples of the aromatic hydrocarbon group include a phenylene group and a naphthylene group.

Examples of the substituent that the cyclic hydrocarbon group optionally has include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl groups having 2 to 4 carbon atoms, an alkylcarbonyloxy group having 2 to 4 carbon atoms, an acryloyloxy group, and a methacryloyloxy group. Examples of these groups include the same groups as those exemplified for R^{a17} in formula (a1-4).

Examples of the ring formed by bonding R^{a62} and R^{a63} each other include an adamantane ring, a cyclopentane ring, or a cyclohexane ring. Specifically, when R^{a62} and R^{a63} are bonded together to form a ring, examples of —C(R^{a62})(R^{a63})(R^{a64}) include the following groups. * represents a bonding site to an oxygen atom. The number of carbon atoms in the ring is preferably 3 to 16, more preferably 3 to 12.

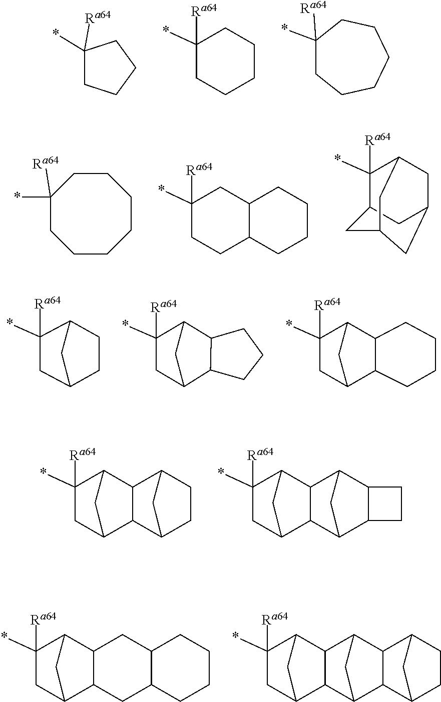

Examples of the alkanediyl group having 1 to 4 carbon atoms in L^{a61} and L^{a62} include a methylene group, an ethylene group, a propane-1,3-diyl group, a propane-1,2-diyl group, a butane-1,4-diyl group, a butane-1,3-diyl group, a 2-methylpropane-1,3-diyl group, and a 2-methylpropane-1,2-diyl group.

It is preferable that L^{a61} and L^{a62} are each independently a methylene group or an ethylene group.

X^{a61} is preferably a single bond or —CO—O—*, and more preferably a single bond.

X^{a62} is preferably a single bond or *—O-L^{a61}, and more preferably a single bond.

Examples of the aromatic hydrocarbon groups having 6 to 20 carbon atoms of Ar include a phenylene group, a 1-naphthylene group, a 2-naphthylene group, a 1-anthrylene group, a 9-anthrylene group, a biphenylene group, a 1-phenanthrylene group, and a 2-phenanthrylene group.

Examples of the substituent that the aromatic hydrocarbon group optionally has include a halogen atom, a hydroxy group, an alkyl group having 1 to 6 carbon atoms, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group. Examples of these groups include the same groups as those exemplified for R^{a17} in formula (a1-4).

Among these, the substituent is preferably a halogen atom, a hydroxy group, an alkyl group having 1 to 4 carbon atoms, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and further preferably a fluorine atom, an iodine atom, a hydroxy group, a methyl group, a methoxy group, or an ethoxyethoxy group.

Ar is preferably a phenylene group optionally having a substituent, and more preferably a phenylene group optionally having a hydroxy group.

Examples of the structural unit (a1-6) include the structural units represented by formulas (a1-6-1) to (a1-6-44), the structural units represented by formulas (a1-6-1) to (a1-6-9) and (a1-6-37) to (a1-6-44) are preferable, the structural units represented by formulas (a1-6-1), (a1-6-2), (a1-6-4), (a1-6-5), (a1-6-7), (a1-6-8), and (a1-6-37) to (a1-6-44) are more preferable, and the structural units represented by formulas (a1-6-7), (a1-6-8), and (a1-6-37) to (a1-6-44) are further preferable.

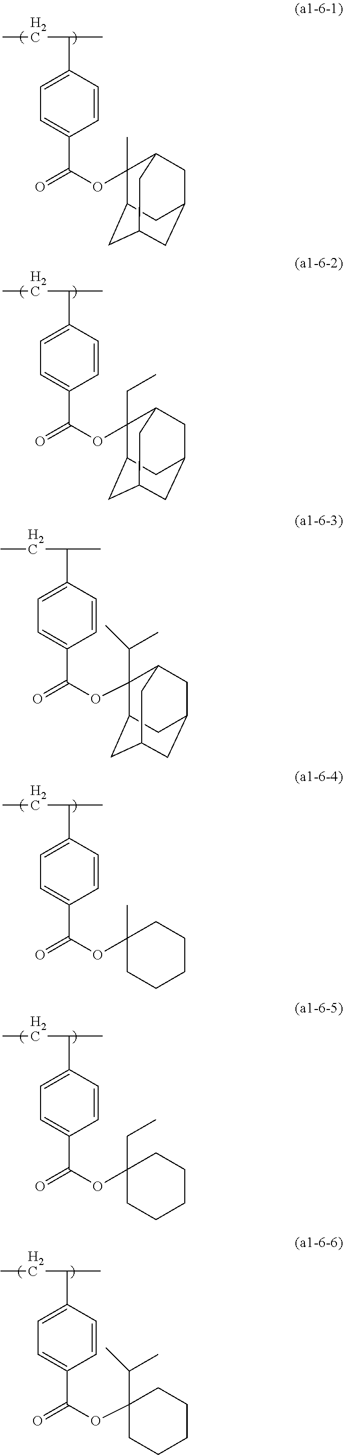

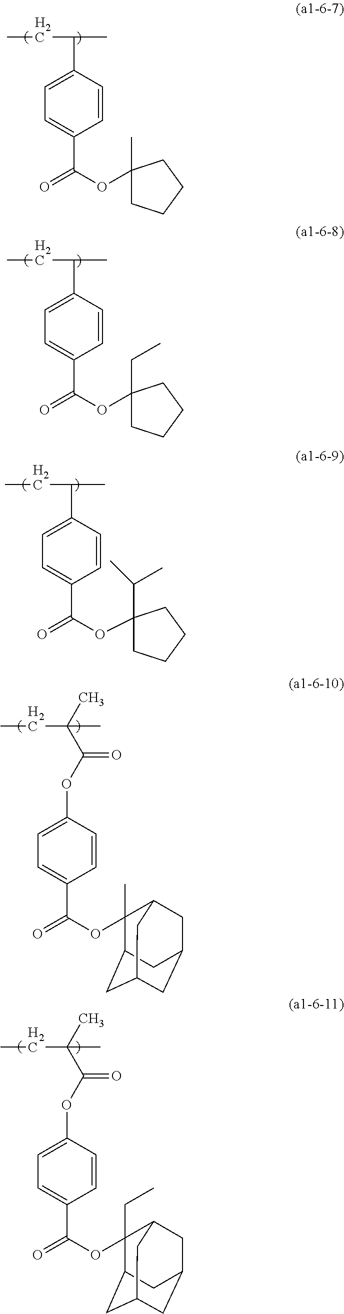

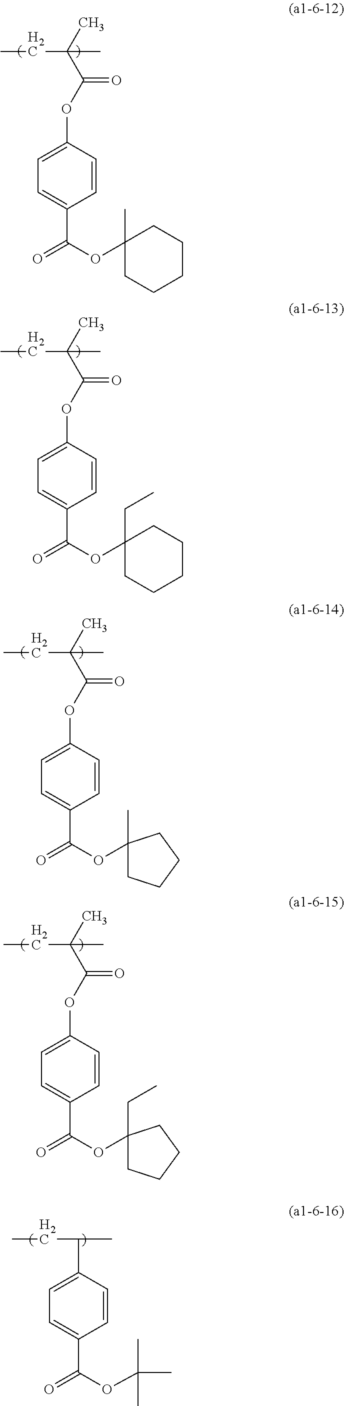

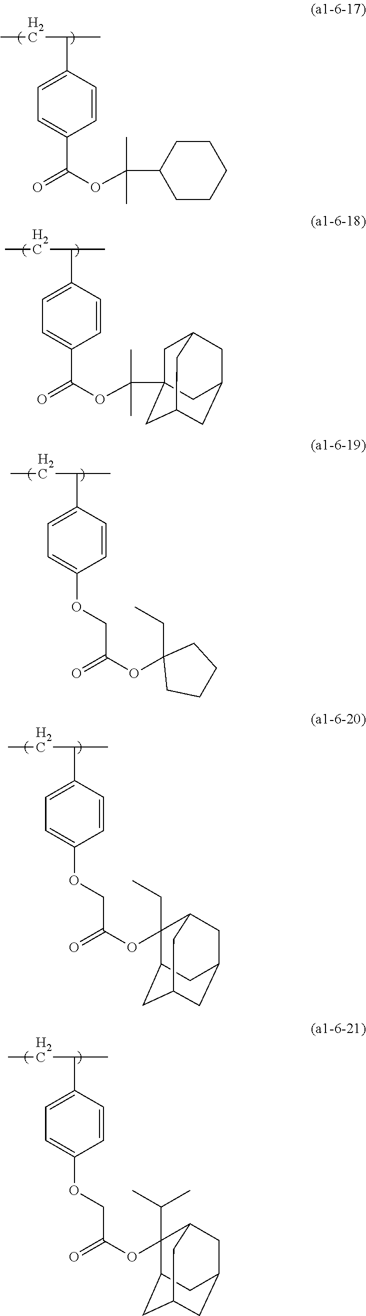

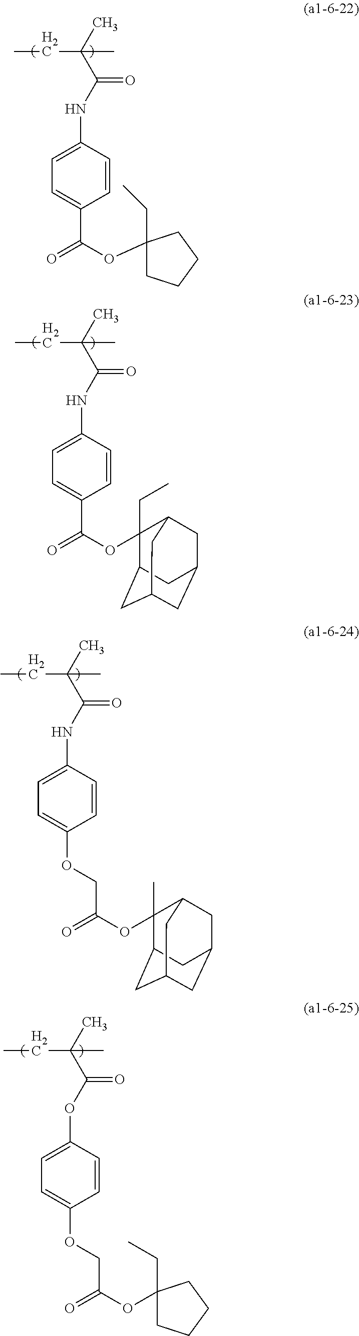

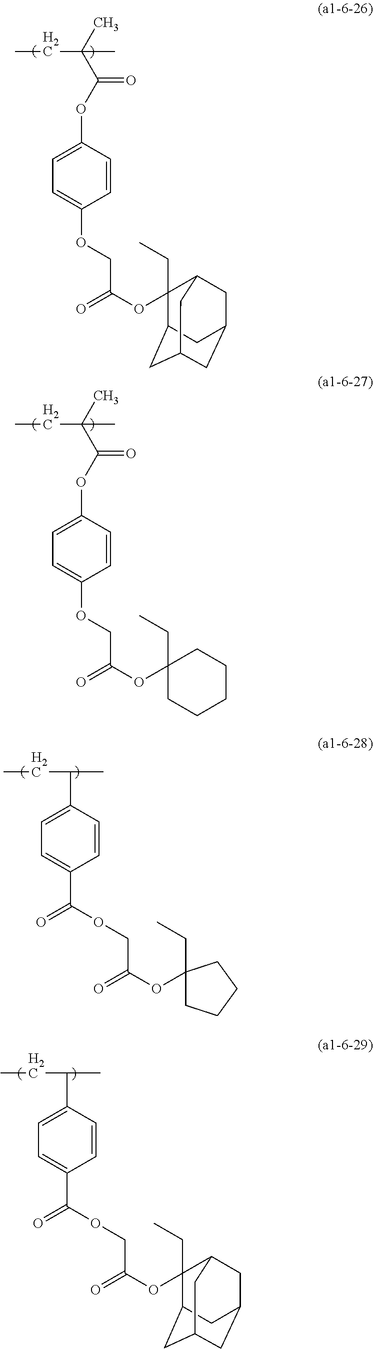

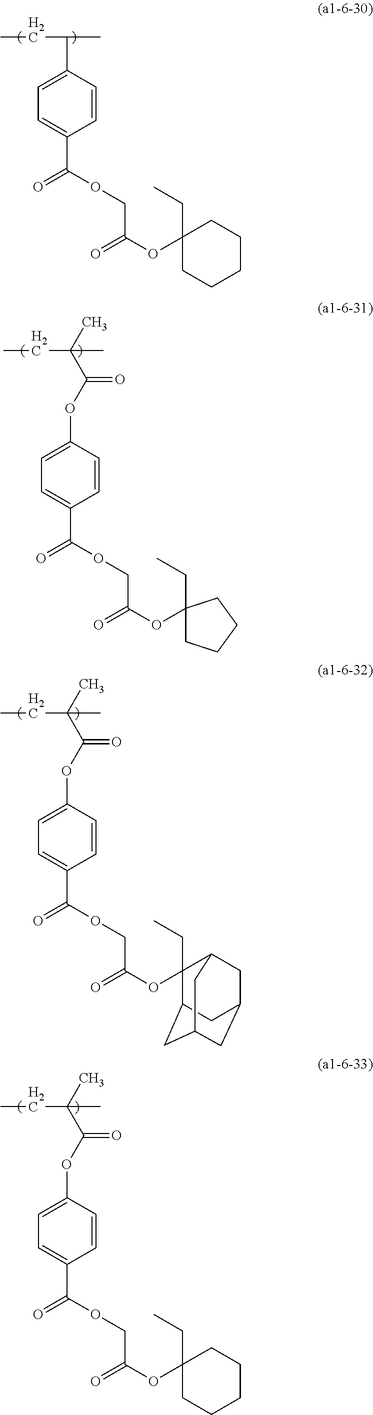

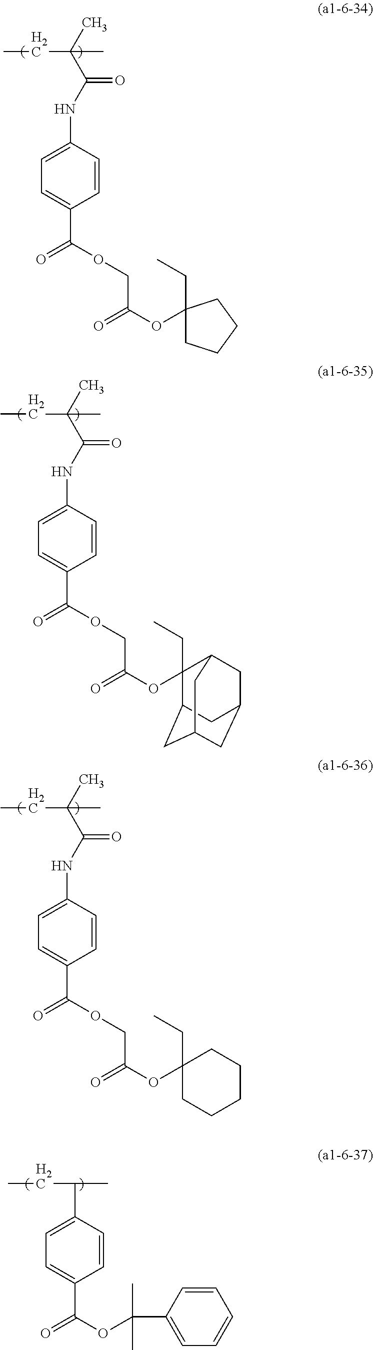

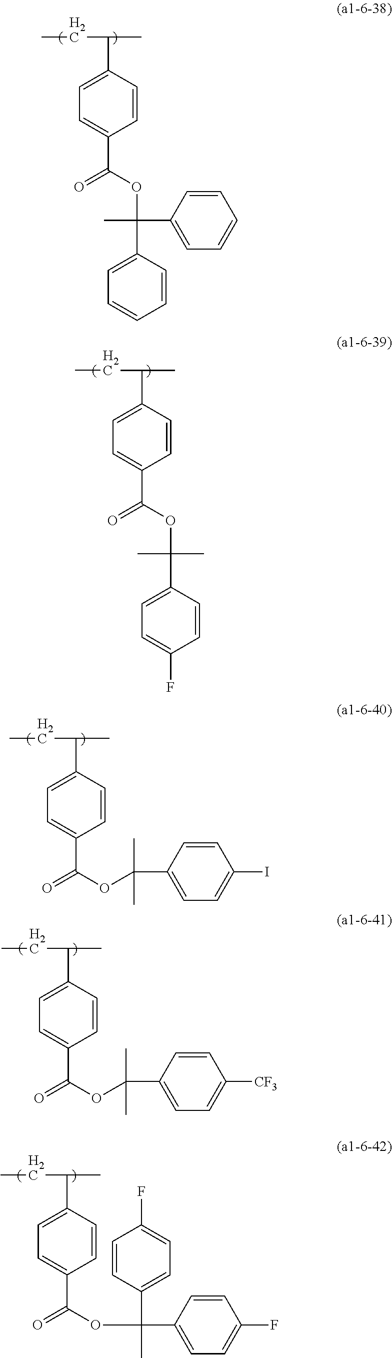

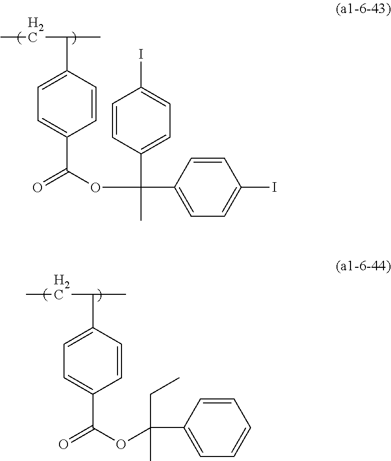

Examples of the structural unit (a1-6) include structural units in which a hydrogen atom corresponding to R^{a61} in the above structural units is replaced with a methyl group or the like, and structural units in which a methyl group corresponding to R^{a61} in the following structural units is replaced with a hydrogen atom etc.

When the resin (A) etc. includes the structural unit (a1-6), the content ratio thereof, with respect to all structural units of the resin (A) etc., 3 mol % or more is exemplified, preferably 5 mol % or more, more preferably 7 mol % or more, further preferably 10 mol % or more, still more preferably 20 mol % or more, even more preferably 30 mol % or more, and most preferably 40 mol % or more. Also, it can be 80 mol % or less, preferably 75 mol % or less, more preferably 70 mol % or less, and even more preferably 65 mol % or less. Specifically, it is preferably 3 to 80 mol %, more preferably 5 to 75 mol %, even more preferably 7 to 70 mol %, even more preferably 7 to 65 mol %, and particularly preferably 10 to 60 mol %.

In addition, examples of the structural unit (a1) include the following structural units.

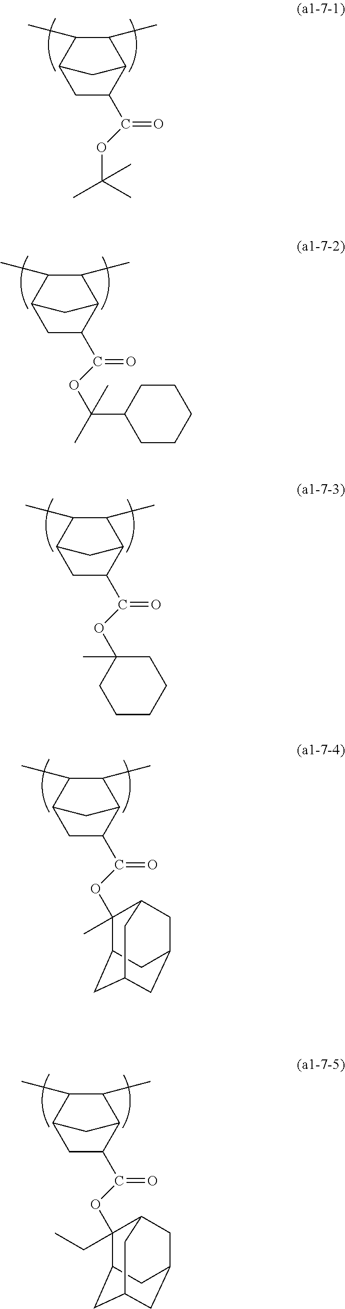

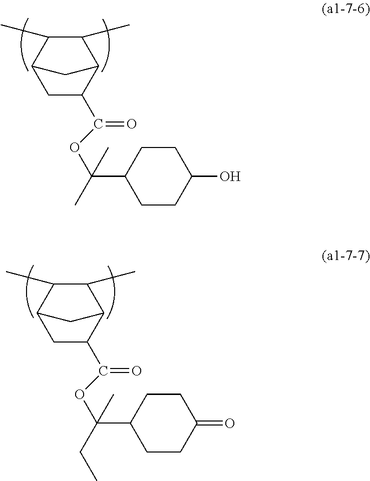

When the resin (A) etc. includes structural units represented by formulas (a1-7-1) to (a1-7-7), the content ratio thereof, with respect to all structural units of the resin (A) etc., 10 mol % or more is exemplified, preferably 15 mol % or more, more preferably 20 mol % or more, further preferably 25 mol % or more, still more preferably 30 mol % or more, even more preferably 40 mol % or more, and most preferably 50 mol % or more. In addition, 95 mol % or less is exemplified, preferably 90 mol % or less, more preferably 85 mol % or less, further preferably 80 mol % or less, still more preferably 75 mol % or less, even more preferably 70 mol % or less, and most preferably 60 mol % or less. Specifically, 10 to 95 mol % is preferable, 15 to 90 mol % is more preferable, 20 to 85 mol % is further preferable, 20 to 70 mol % is still more preferable, and 20 to 60 mol % is particularly preferable.

In addition, examples of the structural unit (a1) include the following structural units.

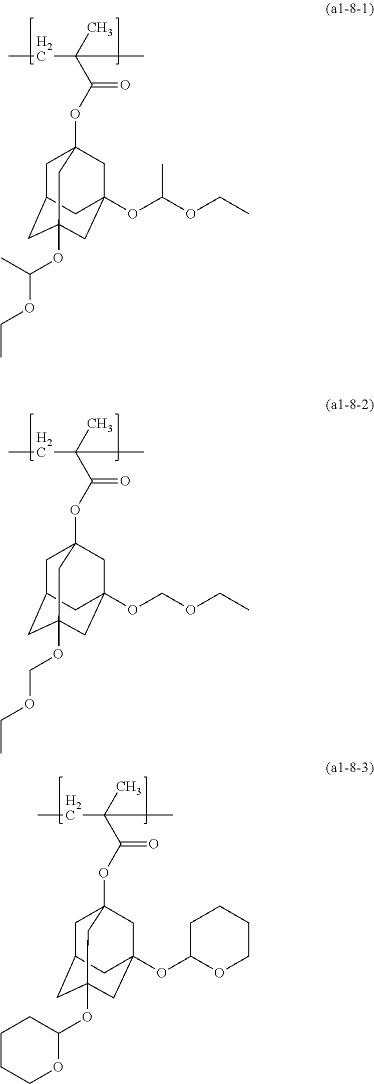

When the resin (A) etc. includes structural units represented by formulas (a1-8-1) to (a1-8-3), the content ratio thereof, with respect to all structural units of the resin (A) etc., 10 mol % or more is exemplified, preferably 15 mol % or more, more preferably 20 mol % or more, further preferably 25 mol % or more, still more preferably 30 mol % or more, even more preferably 40 mol % or more, and most preferably 50 mol % or more. In addition, 95 mol % or less is exemplified, preferably 90 mol % or less, more preferably 85 mol % or less, further preferably 80 mol % or less, still more preferably 75 mol % or less, even more preferably 70 mol % or less, and most preferably 60 mol % or less. Specifically, 10 to 60 mol % is preferable, 15 to 55 mol % is more preferable, 20 to 50 mol % is further preferable, 20 to 45 mol % is still more preferable, and 20 to 40 mol % is particularly preferable.

<Structural Unit (s)>

A structural unit (s) is derived from a monomer that does not have an acid labile group (hereinafter, may be referred to as “monomer (s)”). The monomer from which the structural unit (s) is derived can be a monomer that does not have an acid labile group that is known in the resist field.

The structural unit (s) preferably has a hydroxy group, a carboxy group, or a lactone ring. Using a resin including a structural unit that has a hydroxy group or a carboxy group and no acid labile group (hereinafter may be referred to as “structural unit (a2)”) and/or a structural unit that has a lactone ring and no acid labile group (hereinafter may be referred to as “structural unit (a3)”) in the resist composition of the present invention allows resolution of the resist pattern and adhesion to the substrate to be improved. In addition to the structural units described above, examples of the structural unit (s) include a structural unit that has a halogen atom (hereinafter may be referred to as “structural unit (a4)”), a structural unit that has a non-leaving hydrocarbon group (hereinafter may be referred to as “structural unit (a5)”), a structural unit that has a sultone structure (hereinafter may be referred to as “structural unit (a6)”), a structural unit that decomposes upon exposure to generate acid (hereinafter may be referred to as “structural unit (a7)”), or other structural units known in the art.

<Structural Unit (a2)>

A structural unit (a2) is a structural unit represented by formula (a2) and has an alcoholic hydroxy group, a phenolic hydroxy group, or a carboxy group.

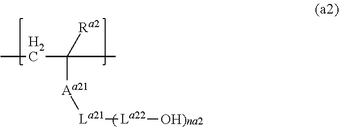

[wherein, in formula (a2),
R^{a2} represents a hydrogen atom, a halogen atom or an alkyl group having 1 to 6 carbon atoms optionally having a halogen atom;
A^{a21} represents a single bond, or an alkanediyl group having 1 to 12 carbon atoms, and —CH₂— contained in the alkanediyl group is optionally replaced with —O—, —CO— or —NR^{a28}—.
R^{a28} represents a hydrogen atom, or an alkyl group having 1 to 6 carbon atoms;
L^{a21} represents a single bond or a hydrocarbon group having 1 to 28 carbon atoms, the hydrocarbon group optionally has a substituent, and —CH₂— contained in the hydrocarbon group is optionally replaced with —O—, —CO—, —S—, —SO—, —NR^{a28}—, or —SO₂—;
L^{a22} represents a single bond or a chain hydrocarbon group having 1 to 12 carbon atoms and optionally having a fluorine atom, and —CH₂— contained in the chain hydrocarbon group is optionally replaced with —O— or —CO—; and
na2 represents an integer of 1 to 5, and When na2 is 2 or more, a plurality of L^{a22} may be the same or different from each other.]

Examples of R^{a2} include the same as those for R^{a1} in formula (a1-4).

Examples of A^{a21} and R^{a28} include the same as A^{a11} and R^{a18} in formula (a1-4).

The hydrocarbon group in L^{a21} is a (na2+1)-valent hydrocarbon group, and examples thereof include a straight-chain or branched chain hydrocarbon group, a monocyclic or polycyclic (including a spiro ring, a condensed ring or bridged ring) alicyclic hydrocarbon group, and a cyclic hydrocarbon group such as an aromatic hydrocarbon group, and may be the combined group of two or more of these groups (for example, a hydrocarbon group formed from an alicyclic hydrocarbon group or an aromatic hydrocarbon group and a chain hydrocarbon group).

Examples of the chain hydrocarbon group in L^{a21} include a divalent to hexavalent chain hydrocarbon group such as an alkanediyl group, an alkanetriyl group, an alkanetetrayl group, an alkanepentyl group, and an alkanehexyl groups.

Examples of the alkanediyl group include the same alkanediyl group as A^{a21} Examples of the alkanetriyl group include a methanetriyl group, an ethanetriyl group, a propanetriyl group, a butanetriyl group, a pentanetriyl group, a hexanetriyl group, a heptanetriyl group, an octanetriyl group, a nonanetriyl group, a decanetriyl group, an undecanetriyl group, a dodecanetriyl group, a tridecanetriyl group, a tetradecanetriyl group, a pentadecanetriyl group, a hexadecanetriyl group, and a heptadecanetriyl group.

Examples of the alkanetetrayl group include a methanetetrayl group, an ethanetetrayl group, a propanetetrayl group, a butanetetrayl group, a pentanetetrayl group, a hexanetetrayl group, a heptanetetrayl group, an octanetetrayl group, a nonanetetrayl group, a decanetetrayl group, an undecanetetrayl group, a dodecanetetrayl group, a tridecanetetrayl group, a tetradecanetetrayl group, a pentadecanetetrayl group, a hexadecanetetrayl group, and a heptadecanetetrayl group.

Examples of the alkanepentyl group include an ethanepentyl group, a propanepentyl group, a butanepentyl group, a pentanepentyl group, and a hexanepentyl group.

Examples of alkanehexyl group include an ethanehexyl group, a propanehexyl group, a butanehexyl group, a pentanehexyl group, and a hexanehexyl group.

In addition, examples thereof include the group formed by replacing one or more of the hydrogen atoms of the above groups with a bonding site.

The number of carbon atoms in the chain hydrocarbon group is preferably 1 to 18, more preferably 1 to 12, further preferably 1 to 10, still more preferably 1 to 9, even more preferably 1 to 8, further more preferably 1 to 6, particularly preferably 1 to 5, and most preferably 1 to 4.

Examples of the monocyclic and polycyclic alicyclic hydrocarbon group in L^{a21} include the following alicyclic hydrocarbon groups. The bonding site can be set to any position.

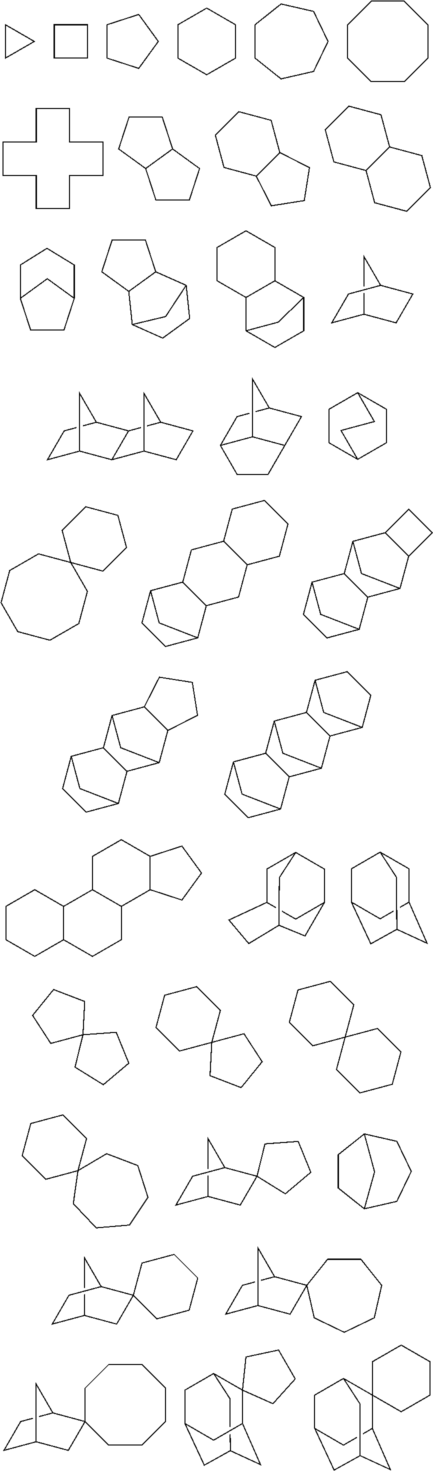

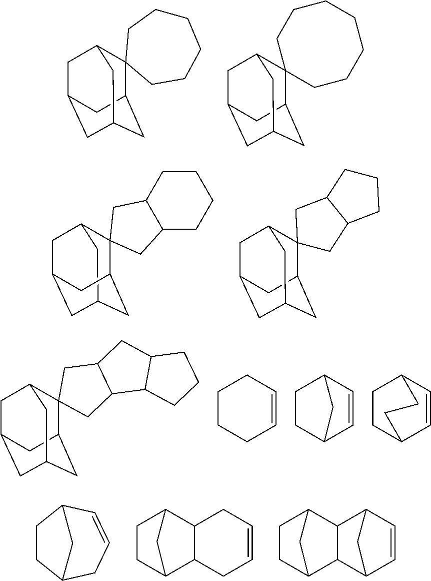

Examples thereof include divalent to hexavalent alicyclic hydrocarbon groups such as a cycloalkanediyl group, a cycloalkanetriyl group, a cycloalkanetetrayl group, a cycloalkanepentyl group, and a cycloalkanehexyl group.

Specific examples thereof include the monocyclic alicyclic hydrocarbon group such as a cyclobutane-1,3-diyl group, a cyclopentane-1,3-diyl group, a cyclohexane-1,4-diyl group, a cyclooctane-1,5-diyl group, a cyclopropanetriyl group, a cyclobutanetriyl group, a cyclopentanetriyl group, a cyclohexanetriyl group, a cycloheptanetriyl group, a cyclooctanetriyl group, a cyclodecanetriyl group, a cyclopropanetetrayl group, a cyclobutanetetrayl group, a cyclopentanetetrayl group, a cyclohexanetetrayl group, a cycloheptanetetrayl group, a cyclooctanetetrayl group, and a cyclodecanetetrayl group and
the polycyclic alicyclic hydrocarbon group such as a norbornane-1,4-diyl group, a norbornane-2,5-diyl group, an adamantane-1,5-diyl group, an adamantane-2,6-diyl group, a decahydronaphthalenediyl group, a bicyclo[3.3.0]octanediyl group, a norbornanetriyl group, an adamantanetriyl group, a decahydronaphthalenetriyl group, a bicyclo[3.3.0]octanetriyl group, a norbornanetetrayl group, an adamantanetetrayl group, a decahydronaphthalenetetrayl group, and a bicyclo[3.3.0]octanetetrayl group.

In addition, examples thereof include the group formed by replacing one or more of the hydrogen atoms of the above groups with a bonding site.

The number of carbon atoms in the alicyclic hydrocarbon group is preferably 3 to 20, more preferably 3 to 18, further preferably 3 to 16, still more preferably 3 to 12, even more preferably 6 to 12, and particularly preferably 6 to 10.

Examples of the aromatic hydrocarbon group in L^{a21} include divalent to hexavalent aromatic hydrocarbon groups such as an arylene group, an arenetriyl group, an arenetetrayl group, an arenepentyl group, and an arenethexyl group.

Specific examples thereof include a phenylene group, a naphthylene group, an anthrylene group, a biphenylene group, a phenanthrylene group, a benzenetriyl group, a naphthalenetriyl group, an anthracenetriyl group, a biphenylenetriyl group, a phenanthrenetriyl group, a benzenetetrayl group, a naphthalenetetrayl group, an anthracenetetrayl group, a biphenylenetetrayl group, and a phenanthrenetetrayl group.

In addition, examples thereof include the group formed by replacing one or more of the hydrogen atoms of the above groups with a bonding site.

The number of carbon atoms in the aromatic hydrocarbon group is preferably 6 to 20, more preferably 6 to 18, further preferably 6 to 14, still more preferably 6 to 12, and most preferably 6 to 10.

Examples of the combined groups of two or more include a combined group of an alicyclic hydrocarbon group and a chain hydrocarbon group, a combined group of an aromatic hydrocarbon group and a chain hydrocarbon group, a combined group of an alicyclic hydrocarbon group and an aromatic hydrocarbon group, and a combined group of an alicyclic hydrocarbon group and a chain hydrocarbon group and an aromatic hydrocarbon group. In the combination, two or more types of alicyclic hydrocarbon groups, aromatic hydrocarbon groups, and chain hydrocarbon groups may each be combined. In addition, any group may be bonded to A^{a21} and L^{a22}.

Examples of the group formed by replacing —CH₂— in a hydrocarbon group with —O—, —S—, —SO₂—, —SO—, —NR^{a28}—, or —CO— include a hydroxy group, a carboxy group, a thiol group, an amino group, an alkoxy group, an alkylthio group, an alkoxycarbonyl group, an alkylsulfonyl group, an alkylamino group, an alkyl peptide group, an alkylcarbonyl group, an alkylcarbonyloxy group, an alkoxycarbonyloxy group, an alkoxyalkoxy group, an oxy group, a carbonyl group, a thio group, a sulfonyl group, a peptide group, an alkanediyloxy group, an alkanediyloxycarbonyl group, an alkanediylcarbonyl group, an alkanediylcarbonyloxy group, an alkanediyloxycarbonyloxy group, an alkanediylsulfonyl group, an alkanediylthio group, an alkanediylamino group, and an alkanediyl peptide group, a cycloalkoxy group, a cycloalkylalkoxy group, an aromatic hydrocarbon group-carbonyloxy group, an aromatic hydrocarbon group-carbonyl group, an aromatic hydrocarbon group-oxy group, and an combined group of two or more of these groups. In addition, examples thereof include the group formed by replacing one or more hydrogen atoms of these groups with a bonding site. Examples of these replaced groups include the same groups as those exemplified in the present specification, within the acceptable range of the upper limit of the number of carbon atoms.

In addition, examples of the group formed by replacing —CH₂— contained in the alicyclic hydrocarbon group, the aromatic hydrocarbon group, or the combined group of these groups with —O—, —S—, —CO—, —SO—, —NR^{a28}— or —SO₂— include the same groups as those exemplified in the present specification, within the acceptable range of the upper limit of the number of carbon atoms.

The hydrocarbon group for L^{a21} optionally has one or more substituents. Examples of the substituents include a halogen atom, a haloalkyl group having 1 to 12 carbon atoms, an alkyl group having 1 to 16 carbon atoms (—CH₂— contained in the alkyl group is optionally replaced with —O— or —CO—), an acryloyloxy group, or a methacryloyloxy group.

Examples of the halogen atom include a fluorine atom, a chlorine atom, a bromine atom and an iodine atom.

The hydrocarbon group for L^{a21} can substantially have a substituent such as a haloalkyl group by having a halogen atom as a substituent. Examples of the haloalkyl group include a fluorinated alkyl group, a chlorinated alkyl group, a brominated alkyl group, and an iodinated alkyl group, and specific examples thereof include a chloromethyl group, a bromomethyl group, an iodomethyl group, a fluoromethyl group, a difluoromethyl group, a trifluoromethyl group, a perfluoroethyl group, a perfluoropropyl group, and a perfluorobutyl group. The number of carbon atoms in the haloalkyl group is preferably 1 to 8, more preferably 1 to 6, further preferably 1 to 4, and still more preferably 1 to 3.

The hydrocarbon group for L^{a21} includes a branched structure, thereby allowing L^{a21} to substantially have a substituent such as an alkyl group. Examples of the alkyl group include a methyl group, an ethyl group, a n-propyl group, an isopropyl group, a n-butyl group, a sec-butyl group, a tert-butyl group, a pentyl group, a hexyl group, a heptyl group, a 2-ethylhexyl group, an octyl group, a nonyl group, a decyl group, an undecyl group, and a dodecyl group. The number of carbon atoms in the alkyl group is preferably 1 to 12, more preferably 1 to 10, further preferably 1 to 8, still more preferably 1 to 6, even more preferably 1 to 4, and even still more preferably 1 to 3.

By the group formed by replacing —CH₂— contained in the hydrocarbon group of L^{a21} with —O—, —S—, —SO—, —NR^{a28}—, —SO₂—, or —CO—, L^{a21} can substantially have substituents such as a hydroxy group, a carboxy group, an alkoxy group, an alkoxycarbonyl group, an alkylcarbonyl group, an alkylcarbonyloxy group, a thiol group, an alkylthio group, an alkylsulfonyl group, an alkoxyalkyl group, an alkoxyalkoxy group, an alkoxycarbonyloxy group, an amino group, an alkylamino group, an alkyl peptide group, an acryloyloxy group, and a methacryloyloxy group. Examples of these groups include the same groups as those exemplified in this specification, within the range permitted by the upper limit of the number of carbon atoms.

The substituent that the hydrocarbon group of L^{a21} optionally has is preferably a halogen atom, a haloalkyl group having 1 to 8 carbon atoms, or an alkyl group having 1 to 12 carbon atoms (—CH₂— contained in the alkyl group is optionally replaced with —O— or —CO—), is more preferably a halogen atom, a haloalkyl group having 1 to 6 carbon atoms, or an alkyl group having 1 to 10 carbon atoms (—CH₂— contained in the alkyl group is optionally replaced with —O— or —CO—), is further preferably a halogen atom, a haloalkyl group having 1 to 4 carbon atoms, an alkyl group having 1 to 4 carbon atoms, a hydroxy group, an alkoxy group having 1 to 4 carbon atoms, or an alkoxyalkoxy group having 2 to 8 carbon atoms, is still more preferably a fluorine atom, an iodine atom, a trifluoromethyl group, a methyl group, a hydroxy group, a methoxy group, an ethoxy group, an ethoxyethoxy group, or an ethoxymethoxy group, and is far more preferably a fluorine atom, an iodine atom, a trifluoromethyl group, a methyl group, a hydroxy group, a methoxy group, or an ethoxyethoxy group.

In addition, when the alkanediyl group of L^{a21} is replaced with —O—, —CO—, or the like, for example, *-L^{a23}-X^{a21}— (L^{a23} represents an alkanediyl group having 1 to 8 carbon atoms, X^{a21} represents —O—, —O—CO—, —CO—O—, or —O—CO—O—, and * represents the bonding site to A^{a21}) is also preferable.

L^{a21} is preferably a single bond, a chain hydrocarbon group having 1 to 12 carbon atoms and optionally having a substituent (wherein —CH₂— contained in the chain hydrocarbon group is optionally replaced with —O—, —CO—, or the like), a cyclic hydrocarbon group having 3 to 20 carbon atoms and optionally having a substituent (wherein —CH₂— contained in the cyclic hydrocarbon group is optionally replaced with —O—, —S—, —CO—, —SO—, —NR^{a28}—, or —SO₂—), or a group formed by combining a chain hydrocarbon group having 1 to 8 carbon atoms and optionally having a substituent and a cyclic hydrocarbon group having 3 to 20 carbon atoms and optionally having a substituent (wherein —CH₂— contained in the chain hydrocarbon group is optionally replaced with —O— or —CO—, and —CH₂— contained in the cyclic hydrocarbon group is optionally replaced with —O—, —S—, —SO₂—, —SO—, —NR^{a28}—, or —CO—), and more preferably a chain hydrocarbon group having 1 to 10 carbon atoms and optionally having a substituent (wherein —CH₂— contained in the chain hydrocarbon group is optionally replaced with —O— or —CO—), a cyclic hydrocarbon group having 3 to 18 carbon atoms and optionally having a substituent (wherein —CH₂— contained in the cyclic hydrocarbon group is optionally replaced with —O—, —S—, —CO—, —SO—, —NR^{a28}—, or —SO₂—), or a group formed by combining a chain hydrocarbon group having 1 to 6 carbon atoms and optionally having a substituent, and a cyclic hydrocarbon group having 3 to 18 carbon atoms and optionally having a substituent (wherein —CH₂— contained in the chain hydrocarbon group is optionally replaced with —O— or —CO—, and —CH₂— contained in the cyclic hydrocarbon group is optionally replaced with —O—, —S—, —SO₂—, —SO—, —NR^{a28}—, or —CO—).

Examples of the chain hydrocarbon group having 1 to 12 carbon atoms for L^{a22} include the same as those exemplified as the straight-chain or branched alkanediyl groups for L^{a21}. The number of carbon atoms in the chain hydrocarbon group is preferably 1 to 10, more preferably 1 to 8, further preferably 1 to 6, and still more preferably 1 to 4.

Examples of the groups in which —CH₂— contained in the chain hydrocarbon group is replaced with —O—, —S—, —CO—, —SO—, or —SO₂— include the same groups as those exemplified in this specification, within the range permitted by the upper limit of the number of carbon atoms.

L^{a22} optionally has one fluorine atom or two or more fluorine atoms.

na2 is preferably an integer of 1 to 4, and more preferably an integer of 1 to 3.

Examples of the structural unit (a2) when L^{a21} is a single bond or a chain hydrocarbon group include the following structural units. Among the following structural units, structural units in which the methyl group corresponding to R^{a2} is replaced with a hydrogen atom or the like are also suitable structural units for the structural unit (a2) like the following structural units.

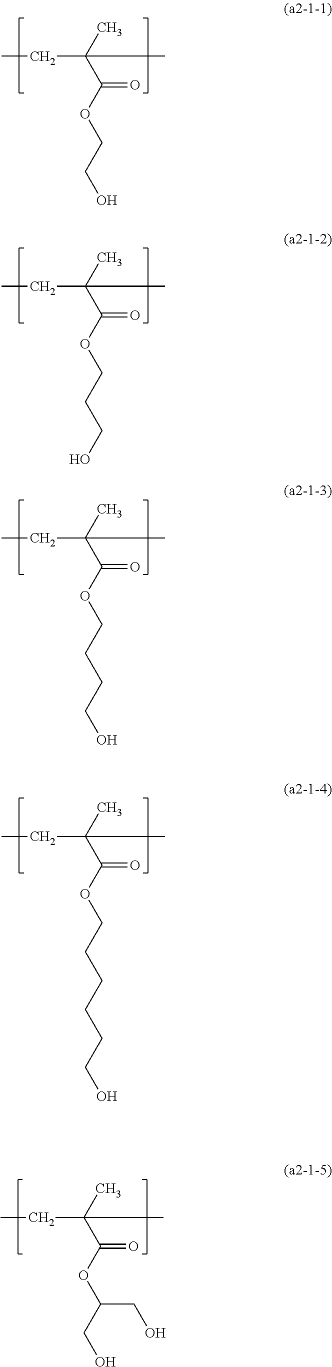

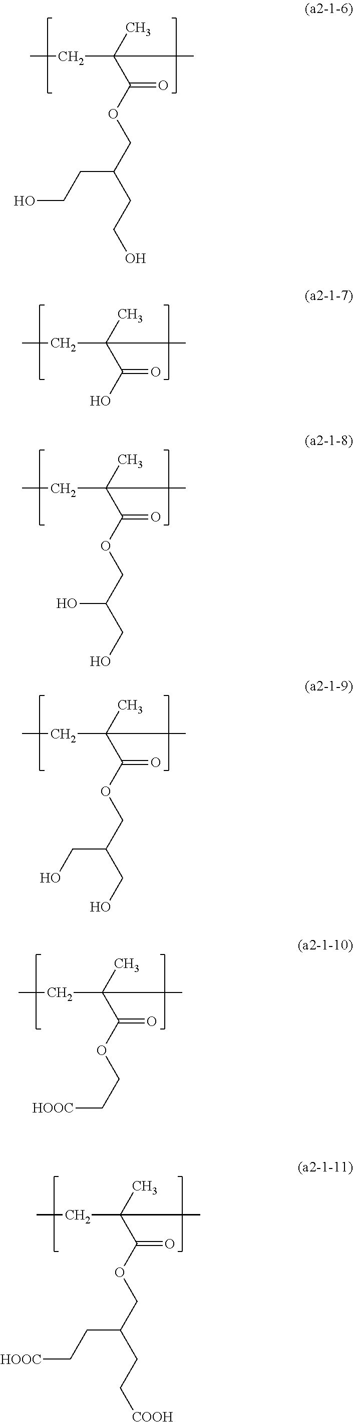

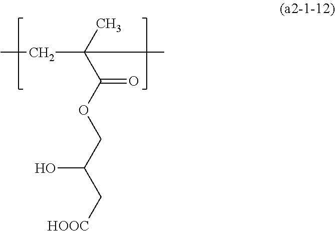

When producing a resist pattern from the resist composition of the present invention and a high-energy ray such as a KrF excimer laser (248 nm), an electron beam, or EUV (extreme ultraviolet) is used as the exposure light source, the structural unit (a2) is preferably a structural unit (a2) having a phenolic hydroxy group, and it is more preferable to use the structural unit (a2-A) described below. In addition, when an ArF excimer laser (193 nm) or the like is used, the structural unit (a2) is preferably a structural unit (a2) having an alcoholic hydroxy group, and it is more preferable to use the structural unit (a2-C) described below. The structural unit (a2) may include one type singly, or two or more types.

In the structural unit (a2), when L^{a21} is a cyclic hydrocarbon group, the structural unit having a phenolic hydroxy group or a carboxy group is a structural unit represented by the formula (a2-A) (hereinafter may be referred to as “structural unit (a2-A)”).

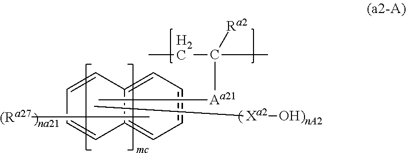

[wherein, in formula (a2-A), all symbols have the same meaning as above.]

R^{a2} and A^{a21} each include the same groups as those exemplified in formula (a2).

Examples of R^{a27} include the same groups as those exemplified for R^{a17} in formula (a1-4).

nA2 is preferably 1, 2, 3, or 4, more preferably 1, 2, or 3, and further preferably 1 or 2.

na21 is preferably 0, 1, 2, or 3, more preferably 0, 1 or 2, and further preferably 0 or 1.

me is preferably 0 or 1.

In the case of a benzene ring, —X^{a2}—OH may be bonded to any of the o-position, m-position, or p-position with respect to the bonding position of A^{a21}. Among these, at least one is preferably bonded to the m-position or p-position, and more preferably bonded to the m-position. In the case of a naphthalene ring, when the bonding position of A^{a21} is the 1st position, the naphthalene ring may be bonded to any of the 2nd to 8th positions, and when the bonding position of A^{a21} is the 2nd position, the naphthalene ring may be bonded to any of the 1st and 3rd to 8th positions. Among these, when the bonding position of A^{a21} is the 1st position, at least one is preferably bonded to the 3rd to 6th positions, and more preferably bonded to the 3rd or 4th position. When the bonding position of A^{a21} is the 2nd position, the naphthalene ring is preferably bonded to the 4th to 7th positions, and more preferably bonded to the 5th or 6th position.

Examples of the structural unit (a2-A) include structural units derived from monomers disclosed in Japanese Patent Laid-Open No. 2010-204634 and Japanese Patent Laid-Open No. 2012-12577.

Examples of the structural unit (a2-A) include structural units represented by formulas (a2-2-1) to (a2-2-32) and (a2-3-1) to (a2-3-24), and structural units represented by formulas (a2-2-1) to (a2-2-32) and (a2-3-1) to (a2-3-24) in which a methyl group corresponding to R^{a2} in the structural unit (a2-A) is replaced with a hydrogen atom, a halogen atom, a haloalkyl group, or another alkyl group.

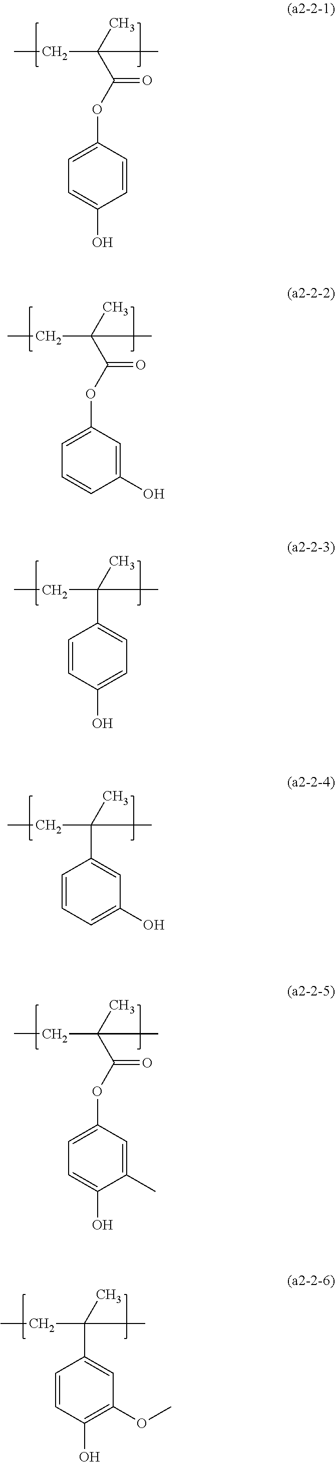

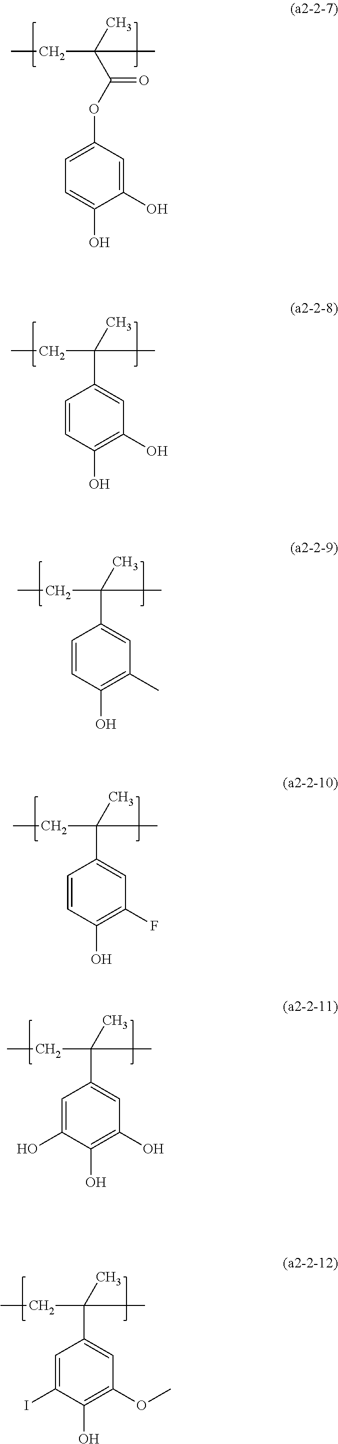

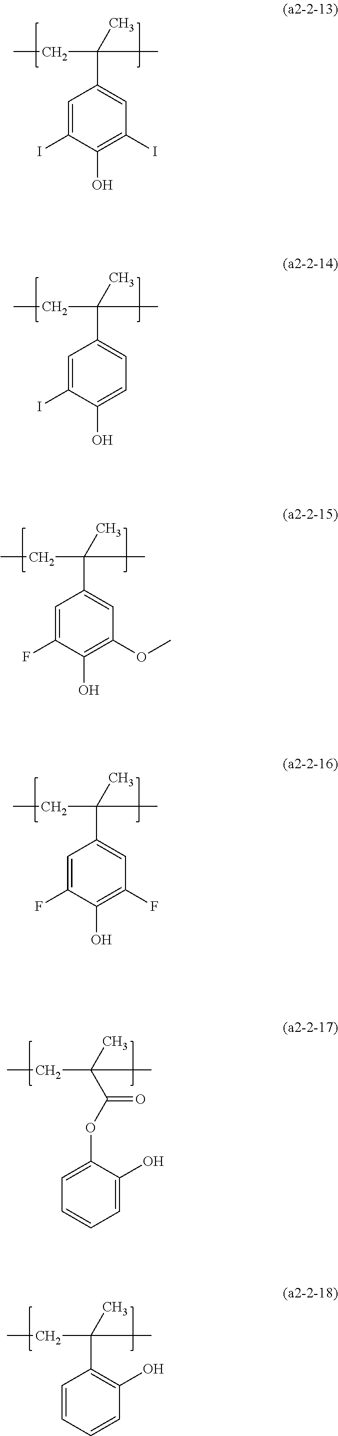

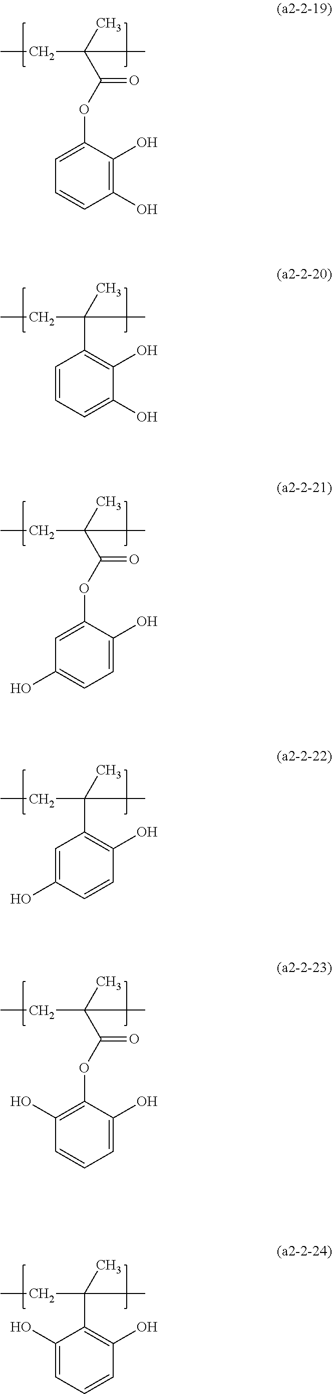

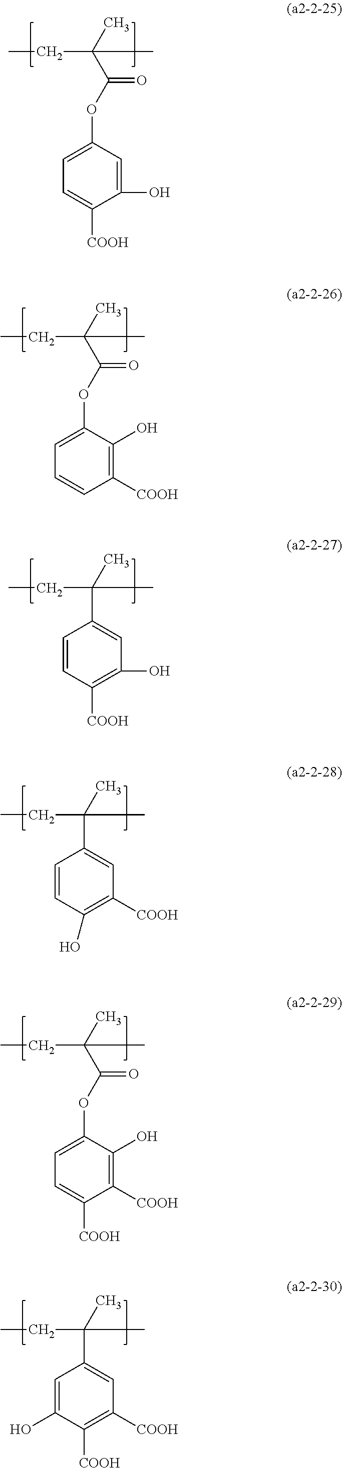

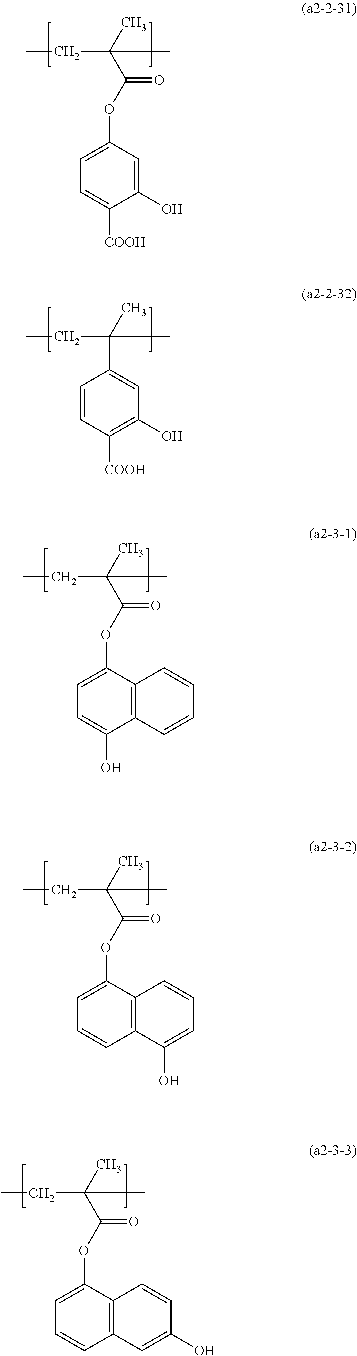

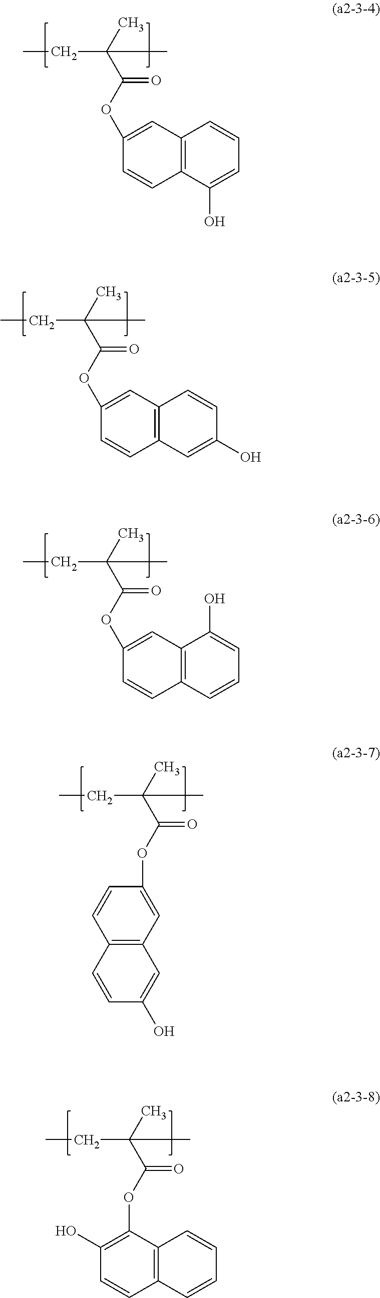

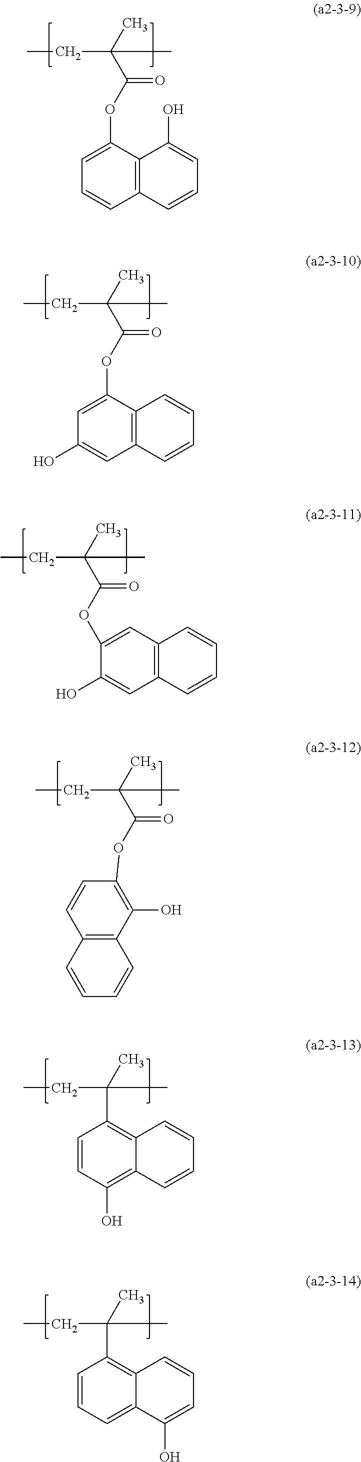

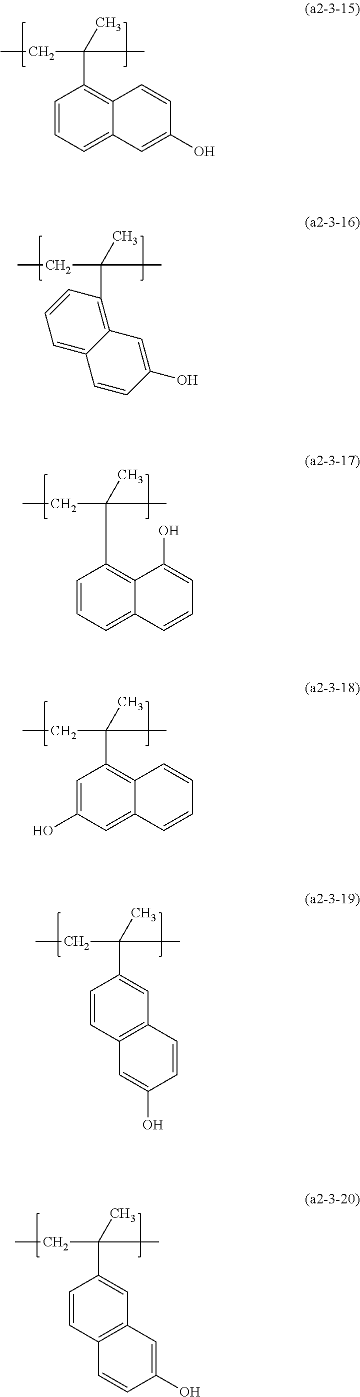

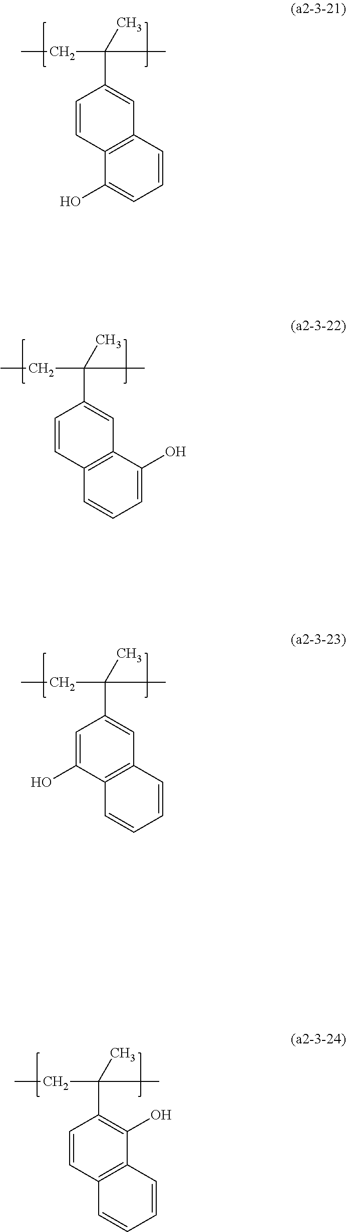

When the structural unit (a2-A) is included in the resin (A) etc., the content ratio of the structural unit (a2-A) with respect to all structural units is preferably 5 mol % or more, more preferably 10 mol % or more, further preferably 15 mol % or more, and still more preferably 20 mol % or more. In addition, the content ratio is preferably 80 mol % or less, more preferably 70 mol % or less, further preferably 65 mol % or less, still more preferably 60 mol % or less, even more preferably 50 mol % or less, further more preferably 45 mol % or less, and most preferably 40 mol % or less. Specifically, the content ratio is preferably 5 to 80 mol %, more preferably 10 to 70 mol %, further preferably 15 to 65 mol %, and still more preferably 20 to 65 mol %.

The structural unit (a2-A) is derived from a compound represented by formula (a2-A′) (hereinafter may be referred to as “compound (a2-A)”).

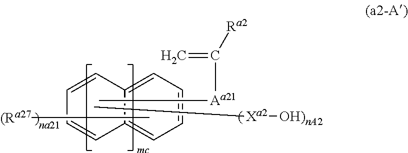

[wherein, in formula (a2-A′), all symbols have the same meaning as above.]

The compound (a2-A′) may be a commercially available product or may be one produced by a known method.

The structural unit (a2-A) can be incorporated into the resin (A) etc. by polymerizing the compound (a2-A′). In addition, for example, the structural unit (a1-4) can be polymerized, followed by treatment with an acid such as p-toluenesulfonic acid, to incorporate the structural unit (a2-A) into the resin (A) etc. In addition, polymerization is performed by using acetoxystyrene and the like, and then treatment is performed with an alkali such as tetramethylammonium hydroxide, thereby allowing the structural unit (a2-A) to be incorporated into the resin (A) etc.

In the structural unit (a2), when L^{a21} is a cyclic hydrocarbon group, examples of the structural unit having an alcoholic hydroxy group or a carboxy group include a structural unit represented by formula (a2-B) (hereinafter may be referred to as “structural unit (a2-B)”), a structural unit represented by formula (a2-C) (hereinafter may be referred to as “structural unit (a2-C)”), and a structural unit represented by formula (a2-D) (hereinafter may be referred to as “structural unit (a2-D)”).

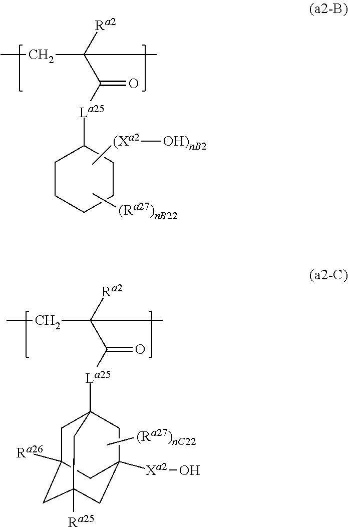

[wherein, in formula (a2-B) and formula (a2-C),
R^{a2} has the same meaning as in formula (a2);
R^{a27} has the same meaning as in formula (a2-A);
L^{a25} represents —O— or *—O—(CH₂)ₖ₂—CO—O—, where k2 represents an integer of 1 to 7, and * represents the bonding site to —CO—;
X^{a2} represents a single bond or —CO—;
R^{a25} and R^{a26} each independently represent a hydrogen atom, a methyl group, or a hydroxy group;
nB2 represents an integer of 1 to 5, and when nB2 is 2 or more, a plurality of X^{a2} may be the same or different from each other;
nB22 represents an integer of 0 to 8, and when nB22 is 2 or more, a plurality of R^{a27} may be the same or different from each other; and
nC22 represents an integer of 0 to 10, and when nC22 is 2 or more, a plurality of R^{a27} may be the same or different from each other.]

L^{a25} is preferably —O— or —O—(CH₂)_{fl}—CO—O— (wherein fl represents an integer of 1 to 4), and more preferably —O—.

R^{a2} is preferably a methyl group.

X^{a2} is preferably a single bond.

R^{a25} is preferably a hydrogen atom.

R^{a26} is preferably a hydrogen atom or a hydroxy group.

R^{a27} is preferably a halogen atom, a hydroxy group, a carboxy group, an alkyl group having 1 to 4 carbon atoms and optionally having a halogen atom, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and still more preferably a fluorine atom, an iodine atom, a hydroxy group, a methyl group, a methoxy group, or an ethoxyethoxy group.

nB2 is preferably 1, 2, 3, or 4, more preferably 1, 2, or 3, and further preferably 1 or 2.

nB22 is preferably an integer of 0 to 3, more preferably 0, 1, or 2, and further preferably 0 or 1.

nC22 is preferably an integer of 0 to 6, more preferably an integer of 0 to 3, further preferably 0, 1 or 2, and still more preferably 0 or 1.

Examples of the structural unit (a2-B) and the structural unit (a2-C) include structural units derived from the monomers disclosed in Japanese Patent Laid-Open No. 2010-204646, the following structural units, and structural units in which the methyl group or hydrogen atom corresponding to R^{a2} in the following structural units has been replaced with a hydrogen atom, a halogen atom, a haloalkyl group, or another alkyl group. Among these, structural units represented by any of formulas (a2-B-1) to (a2-B-5) and formulas (a2-C-1) to (a2-C-9) are preferable.

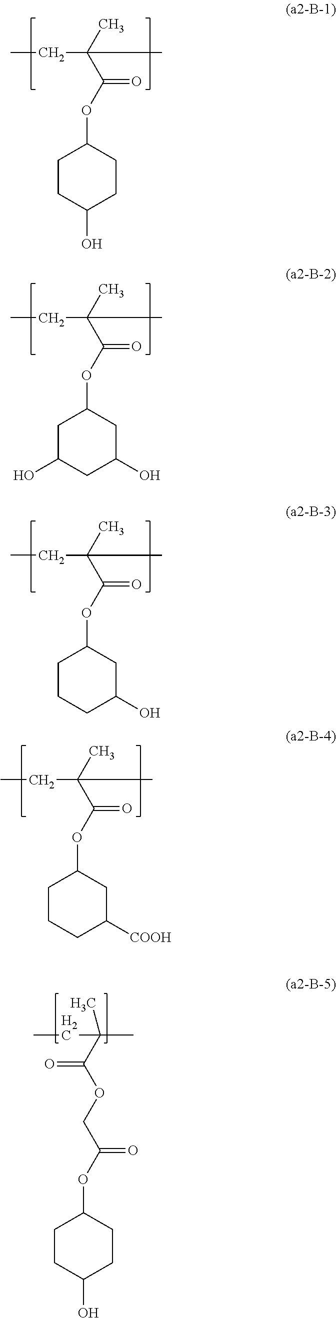

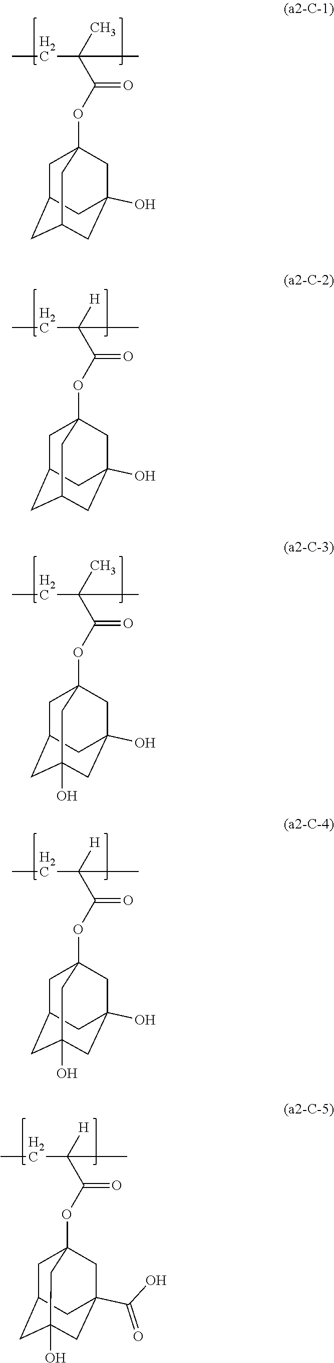

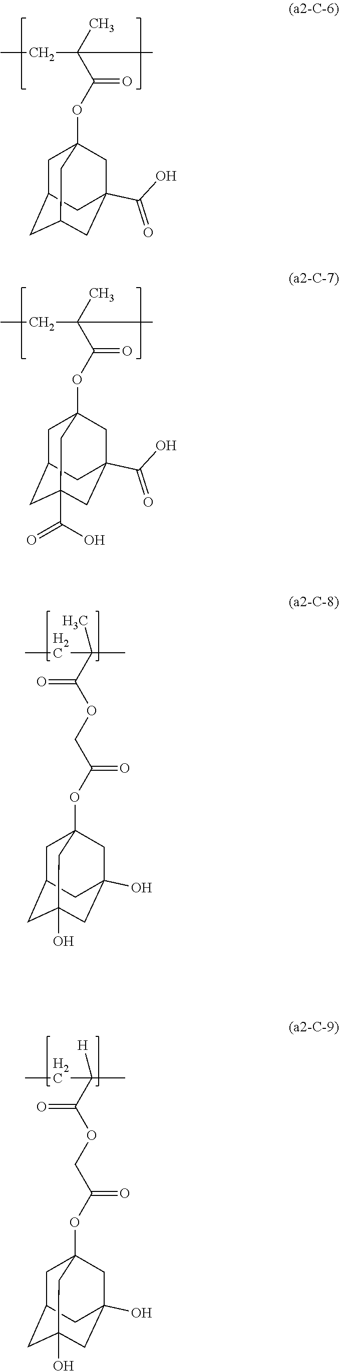

When the resin (A) etc. includes a structural unit (a2-B) or a structural unit (a2-C), the content ratio thereof is 1 mol % or more, and preferably 2 mol % or more, with respect to the total structural units of the resin (A) etc. In addition, 45 mol % or less is exemplified, preferably 40 mol % or less, more preferably 35 mol % or less, further preferably 20 mol % or less, and still more preferably 10 mol % or less. Specifically, 1 to 45 mol % is exemplified, preferably 1 to 40 mol %, more preferably 1 to 35 mol %, further preferably 1 to 20 mol %, and still more preferably 1 to 10 mol %.

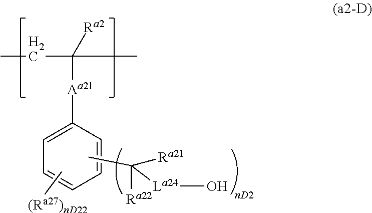

[wherein, in formula (a2-D),
R^{a2} and A^{a21} each have the same meaning as in formula (a2);
R^{a27} has the same meaning as in formula (a2-A);
R^{a21} and R^{a22} each independently represent a fluorinated alkyl group having 1 to 4 carbon atoms;
L^{a24} represents a single bond or an alkanediyl group having 1 to 3 carbon atoms and the alkanediyl group may be substituted with a fluorine atom;
nD2 represents an integer of 1 to 5, and when nD2 is 2 or more, groups in a plurality of parentheses may be the same or different from each other; and
nD22 represents an integer of 0 to 4, and when nD22 is 2 or more, a plurality of R^{a27} may be the same or different from each other,
provided that 1≤nD2+nD22≤5.]

The fluorinated alkyl groups of R^{a21} and R^{a22} each independently include a trifluoromethyl group, a 2,2,2-trifluoroethyl group, a 3,3,3-trifluoropropyl group, a 4,4,4-trifluorobutyl group, and the like.

R^{a21} and R^{a22} are preferably trifluoromethyl groups.

Examples of the alkanediyl group of L^{a24} include a methylene group, an ethane-1,1-diyl group, a propane-1,1-diyl group, and a propane-2,2-diyl group.

L^{a24} is preferably a single bond or a methylene group.

R^{a27} is preferably a halogen atom, a hydroxy group, a carboxy group, an alkyl group having 1 to 4 carbon atoms and optionally having a halogen atom, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and further preferably a fluorine atom, an iodine atom, a hydroxy group, a methyl group, a methoxy group, or an ethoxyethoxy group.

nD2 is preferably 1, 2, 3, or 4, more preferably 1, 2, or 3, further preferably 1 or 2, and still more preferably 1. In addition, preferably, nD2 is 1 and the group in parentheses is bonded to the para position.

nD22 is preferably an integer of 0 to 3, more preferably an integer of 0 to 2, further preferably 0 or 1, and still more preferably 0.

It is more preferable that the structural unit (a2-D) is a structural unit represented by the following formula (a2-D1) (hereinafter, may be referred to as “structural unit (a2-D1)”).

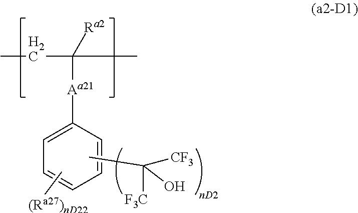

[wherein, in formula (a2-D1),
R^{a2}, A^{a21}, R^{a27}, nD2, and nD22 have the same meaning as in formula (a2-D).]

In formula (a2-D1), R^{a2} is preferably a hydrogen atom or a methyl group.

A^{a21} is preferably a single bond.

R^{a27} is preferably a halogen atom, a hydroxy group, a carboxy group, an alkyl group having 1 to 4 carbon atoms, a haloalkyl group having 1 to 4 carbon atoms, an alkoxy group having 1 to 4 carbon atoms, or an alkoxyalkoxy group having 2 to 8 carbon atoms, more preferably a fluorine atom, an iodine atom, a hydroxy group, a methyl group, a methoxy group, an ethoxy group, an ethoxyethoxy group, or an ethoxymethoxy group, and still more preferably a fluorine atom, an iodine atom, a hydroxy group, a methyl group, a methoxy group, or an ethoxyethoxy group.

nD22 is preferably 0, 1, 2, or 3, more preferably 0, 1, or 2, and further preferably 0 or 1.

nD2 is preferably 1, 2, 3, or 4, more preferably 1, 2, or 3, and further preferably 1 or 2.

Examples of the structural unit (a2-D) include the structural units shown below. In the following structural units, structural units in which a hydrogen atom corresponding to R^{a2} is replaced with a methyl group or the like, and structural units in which a methyl group is replaced with a hydrogen atom or the like can also be exemplified as specific examples of the structural unit (a2-D). Among these, the structural units represented by formulas (a2-D-1) to (a2-D-8) are preferable, the structural units represented by formulas (a2-D-1) to (a2-D-4) are more preferable, and the structural unit represented by formula (a2-D-1) is further preferable.

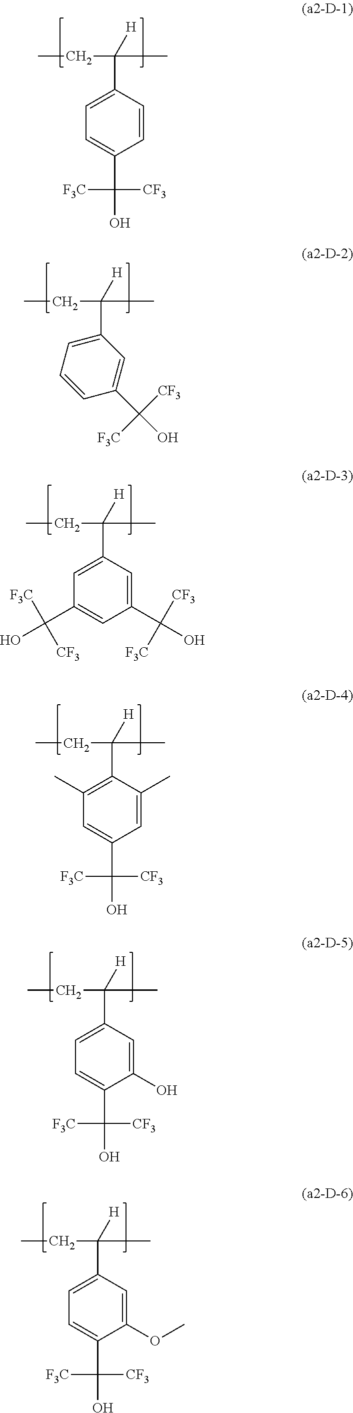

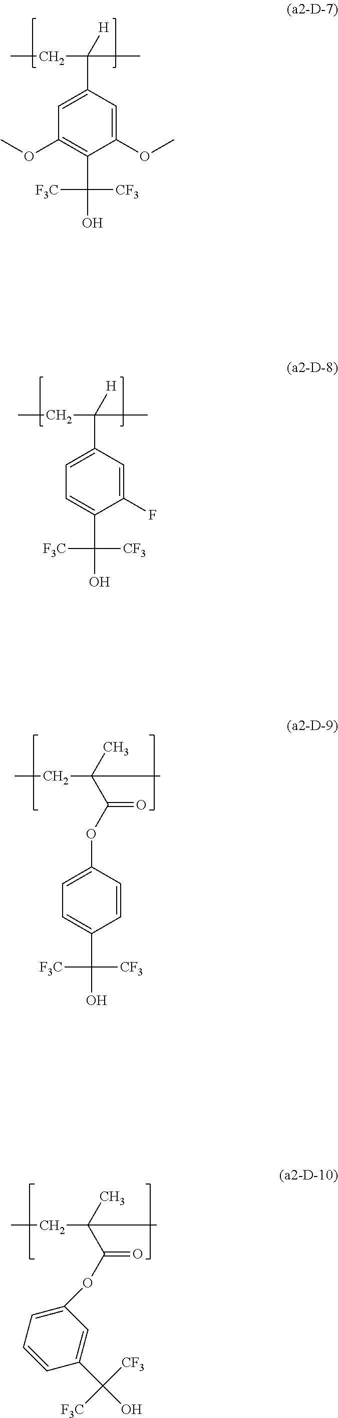

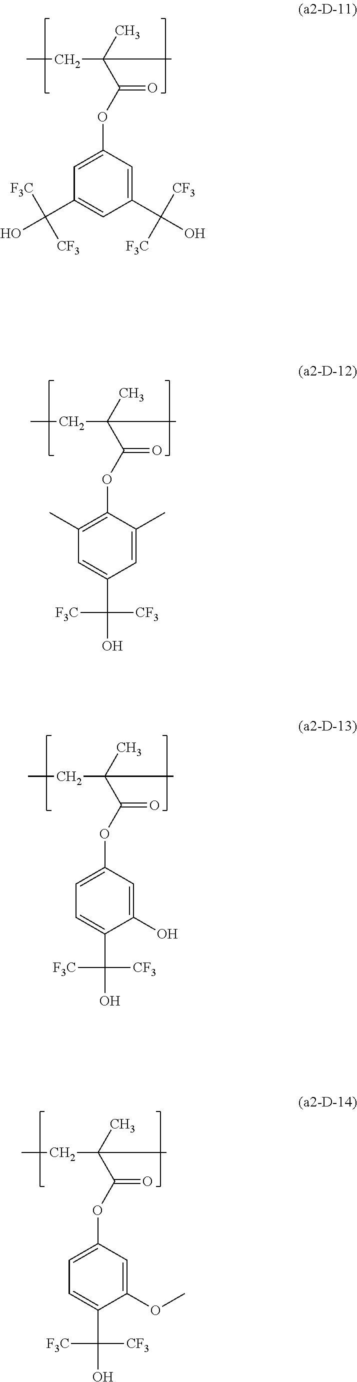

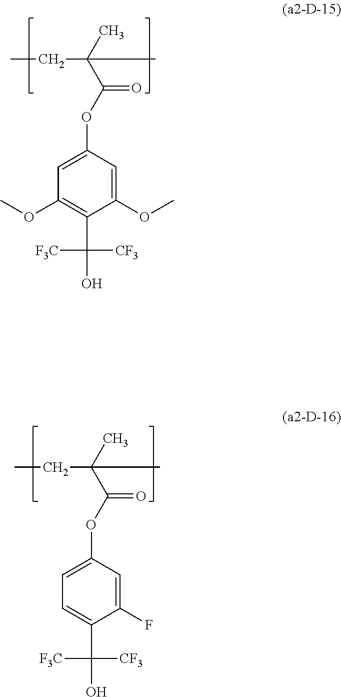

When the resin (A) etc. includes a structural unit (a2-D), the content ratio thereof is preferably 3 mol % or more, more preferably 5 mol % or more, and further preferably 10 mol % or more, with respect to all structural units of the resin (A) etc. In addition, the content ratio is preferably 80 mol % or less, more preferably 75 mol % or less, further preferably 70 mol % or less, and still more preferably 65 mol % or less. Specifically, the content ratio is preferably 3 to 80 mol %, more preferably 5 to 75 mol %, further preferably 10 to 70 mol %, and still more preferably 10 to 65 mol %.

<Structural Unit (a3)>

The lactone ring in a structural unit (a3) may be a monocyclic ring such as a β-propiolactone ring, a γ-butyrolactone ring, or a δ-valerolactone ring, or may be a condensed ring of a monocyclic lactone ring with another ring. Preferable are a γ-butyrolactone ring, an adamantane lactone ring, or a bridged ring including a γ-butyrolactone ring structure (for example, the structural unit represented by the following formula (a3-2)).

The structural unit (a3) is preferably a structural unit represented by formula (a3-1), formula (a3-2), formula (a3-3), or formula (a3-4). One of these may be contained singly, or two or more types may be contained.

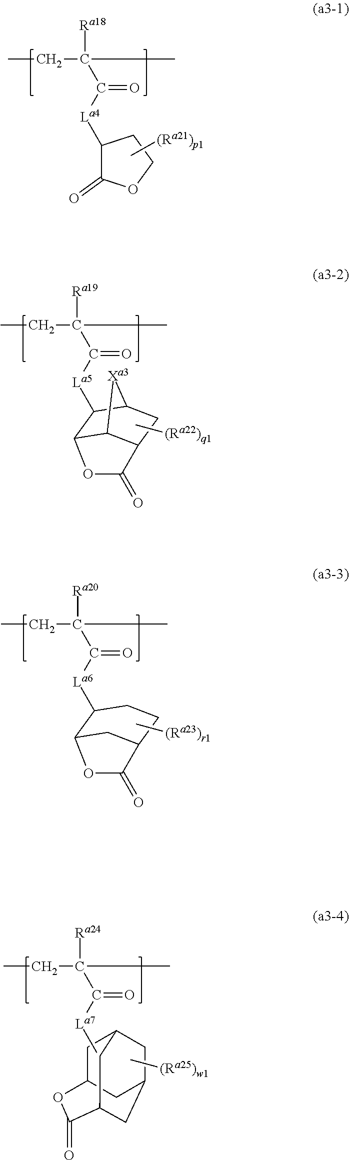

[wherein, in formula (a3-1), formula (a3-2), formula (a3-3), and formula (a3-4),
L^{a4}, L^{a5}, and L^{a6} each independently represent a group represented by —O— or *—O—(CH₂)ₖ₃—CO—O— (k3 represents an integer of 1 to 7);
L^{a7} represents —O—, *—O-L^{a8}-O—, *—O-L^{a8}-CO—O—, *—O-L^{a8}-CO—O-L^{a9}-CO—O—, or *—O-L^{a8}-O—CO-L^{a9}-O—;
L^{a8} and L^{a8} each independently represent an alkanediyl group having 1 to 6 carbon atoms;
* represents the bonding site to the carbonyl group;
R^{a18}, R^{a19}, R^{a20}, and R^{a24} each independently represent a hydrogen atom, a halogen atom, or an alkyl group having 1 to 6 carbon atoms and optionally having a halogen atom;
X^{a3} represents —CH₂— or an oxygen atom;
R^{a21} represents an aliphatic hydrocarbon group having 1 to 4 carbon atoms;
R^{a22}, R^{a23}, and R^{a25} each independently represent a carboxy group, a cyano group, or an aliphatic hydrocarbon group having 1 to 4 carbon atoms;
p1 represents an integer of 0 to 5;
q1 represents an integer of 0 to 3;
r1 represents an integer of 0 to 3;
w1 represents an integer of 0 to 8; and
when p1, q1, r1, and/or w1 are 2 or more, a plurality of R^{a21}, R^{a22}, R^{a23}, and/or R^{a25} may be the same or different from each other.]

The aliphatic hydrocarbon groups in R^{a21}, R^{a22}, R^{a23}, and R^{a25} include alkyl groups such as a methyl group, an ethyl group, a propyl group, an isopropyl group, a butyl group, a sec-butyl group, and a tert-butyl group.

Examples of the halogen atoms and alkyl groups optionally having halogen atoms in R^{a18}, R^{a19}, R^{a20}, and R^{a24} include the same as those exemplified for R^{a01}, R^{a4}, and R^{a5} in formulas (a1-0) to (a1-2).

Examples of the alkanediyl group in L^{a8} and L^{a8} include a methylene group, an ethylene group, a propane-1,3-diyl group, a propane-1,2-diyl group, a butane-1,4-diyl group, a pentane-1,5-diyl group, a hexane-1,6-diyl group, a butane-1,3-diyl group, a 2-methylpropane-1,3-diyl group, a 2-methylpropane-1,2-diyl group, a pentane-1,4-diyl group, and a 2-methylbutane-1,4-diyl group.

L^{a4} to L^{a8} are each independently preferably —O— or *—O—(CH₂)ₖ₃—CO—O—, where k3 is an integer of 1 to 4, more preferably —O— and *—O—CH₂—CO—O—, and further preferably an oxygen atom.

L^{a7} is preferably —O— or *—O-L^{a8}-CO—O—, more preferably —O—, —O—CH₂—CO—O—, or —O—C₂H₄—CO—O—.

R^{a18}, R^{a19}, R^{a20}, and R^{a24} are preferably a hydrogen atom or an alkyl group having 1 to 4 carbon atoms, more preferably a hydrogen atom, a methyl group, or an ethyl group, and further preferably a hydrogen atom or a methyl group.

R^{a21} is preferably a methyl group.

R^{a22}, R^{a23}, and R^{a25} are each independently preferably a carboxy group, a cyano group, or a methyl group.

p1, q1, and r1 are each independently preferably an integer of 0 to 2, more preferably 0 or 1.

w1 is preferably an integer of 0 to 2, more preferably 0 or 1.

In particular, formula (a3-4) is preferably formula (a3-4′).

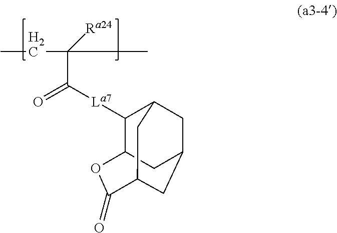

(In the formula, R^{a24} and L^{a7} have the same meanings as above)

Examples of the structural unit (a3) include structural units derived from the monomers disclosed in Japanese Patent Laid-Open No. 2010-204646, Japanese Patent Laid-Open No. 2000-122294, and Japanese Patent Laid-Open No. 2012-41274. Preferable structural unit (a3) is a structural unit represented by any one of formulas (a3-1-1), (a3-1-2), (a3-2-1), (a3-2-2), (a3-3-1), (a3-3-2), and (a3-4-1) to (a3-4-12), as well as the structural units in which the methyl groups corresponding to R^{a18}, R^{a19}, R^{a20}, and R^{a24} in formulas (a3-1) to (a3-4) are replaced with hydrogen atoms.

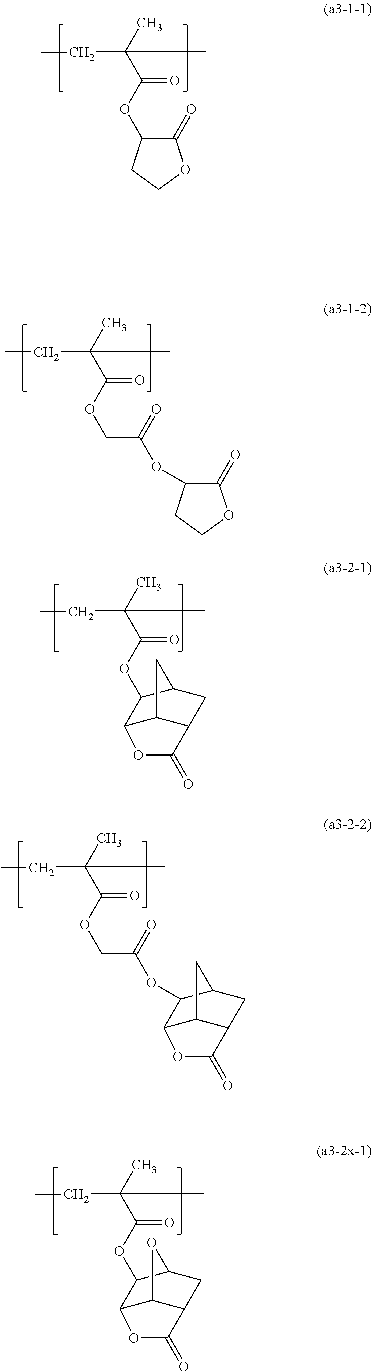

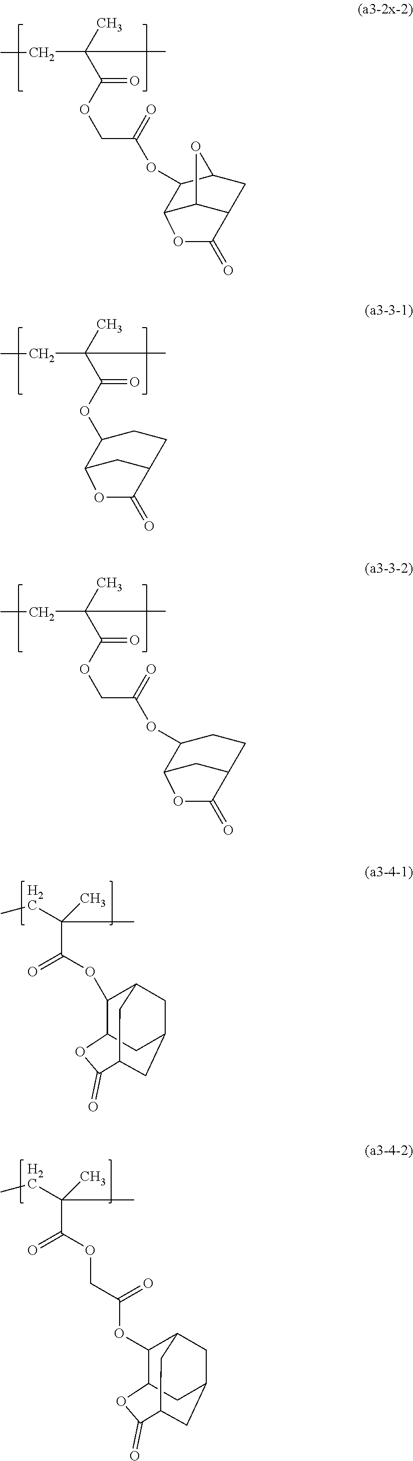

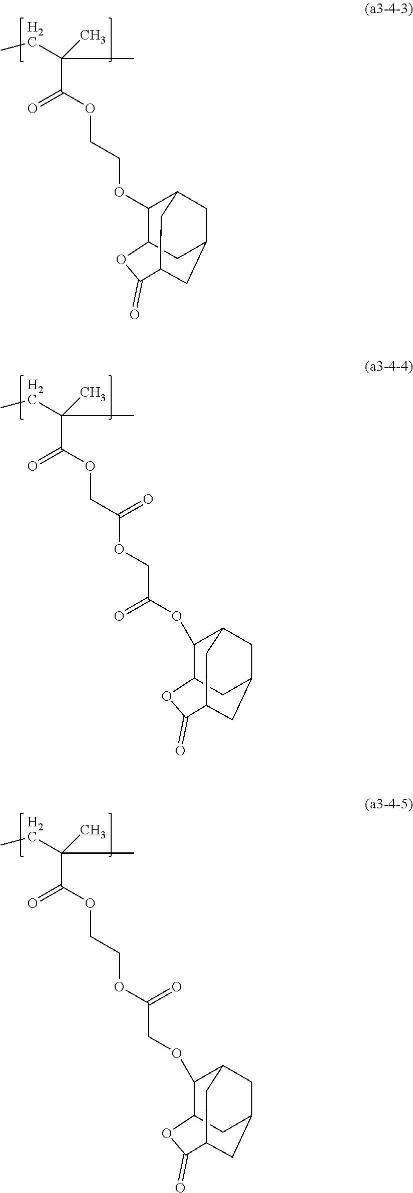

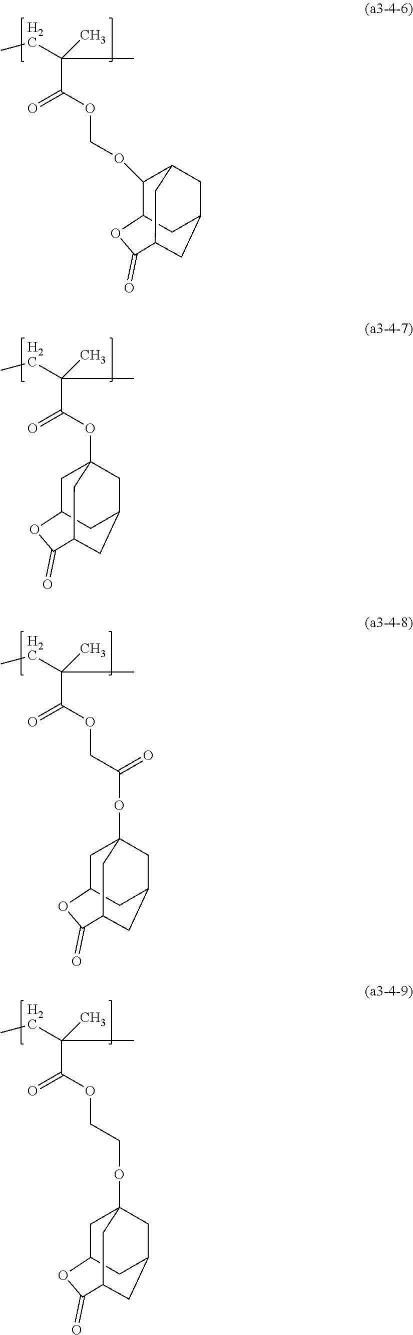

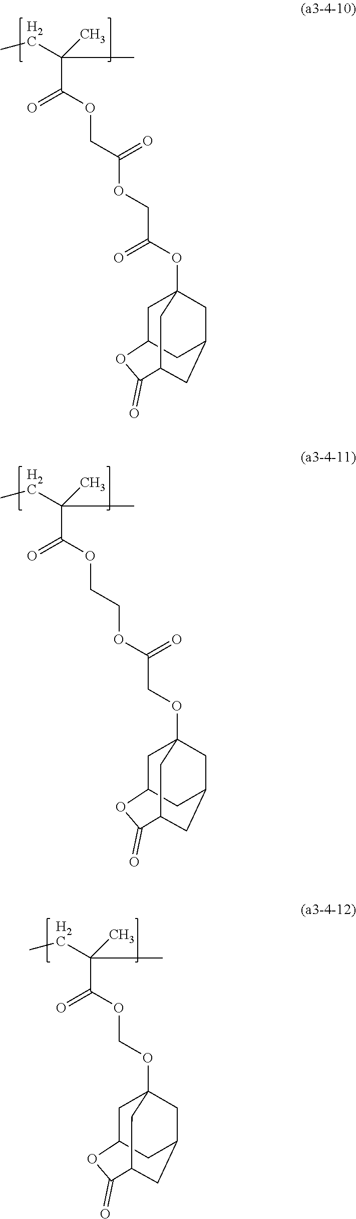

When the resin (A) etc. includes the structural unit (a3), the total content ratio thereof, with respect to all structural units of the resin (A) etc., is 1 mol % or more, preferably 3 mol % or more, more preferably 5 mol % or more, and further preferably 10 mol % or more. In addition, 70 mol % or less is exemplified, preferably 65 mol % or less, more preferably 60 mol % or less, further preferably 50 mol % or less, still more preferably 40 mol % or less, even more preferably 30 mol % or less, further more preferably 25 mol % or less, and most preferably 20 mol % or less. Specifically, 1 to 70 mol % is exemplified, preferably 3 to 70 mol %, more preferably 3 to 65 mol %, further preferably 5 to 65 mol %, still more preferably 5 to 60 mol %, and most preferably 10 to 60 mol %.

In addition, the content ratio of the structural unit (a3-1), the structural unit (a3-2), the structural unit (a3-3), or the structural unit (a3-4) is preferably 1 mol or more, more preferably 3 mol % or more, further preferably 5 mol % or more, and still more preferably 10 mol % or more, with respect to the total structural units of the resin (A) etc. In addition, the content ratio is preferably 60 mol % or less, more preferably 55 mol % or less, further preferably 50 mol % or less, still more preferably 40 mol % or less, even more preferably 30 mol % or less, further more preferably 25 mol % or less, and most preferably 20 mol % or less. Specifically, it is preferably 1 to 60 mol %, more preferably 3 to 60 mol %, further preferably 3 to 55 mol %, still more preferably 5 to 55 mol %, even more preferably 5 to 50 mol %, and further more preferably 10 to 50 mol %.

<Structural Unit (a4)>

The structural unit represented by formula (a4) is shown below.

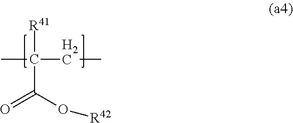

[wherein, in formula (a4),
R⁴¹ represents a hydrogen atom or a methyl group; and
R⁴² represents an aliphatic hydrocarbon group having 1 to 48 carbon atoms and a halogen atom, and —CH₂— contained in the aliphatic hydrocarbon group is optionally replaced with —O— or —CO—.]

Examples of the aliphatic hydrocarbon group represented by R⁴² include straight-chain or branched chain hydrocarbon groups, monocyclic or polycyclic alicyclic hydrocarbon groups, and groups formed by combining these groups.

Examples of the chain hydrocarbon group include a straight-chain alkyl group such as a methyl group, an ethyl group, a propyl group, a butyl group, a pentyl group, a hexyl group, a heptyl group, an octyl group, a decyl group, a dodecyl group, a pentadecyl group, a hexadecyl group, a heptadecyl group, and an octadecyl group, and a branched alkyl group such as an isopropyl group and an isobutyl group.

Examples of the monocyclic or polycyclic alicyclic hydrocarbon group include: a monocyclic alicyclic hydrocarbon group that is a monocyclic cycloalkyl group such as a cyclopentyl group, a cyclohexyl group, a cycloheptyl group, and a cyclooctyl group; and a polycyclic alicyclic hydrocarbon group that is a polycyclic cycloalkyl group such as a decahydronaphthyl group, an adamantly group, and a norbornyl group, and the following groups (* indicates a bonding site).

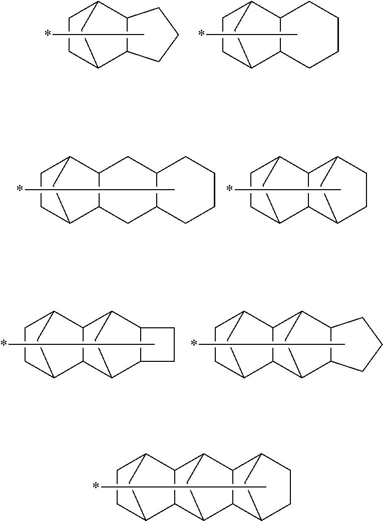

Examples of the groups formed by combination include groups formed by combining one or more alkyl groups or one or more alkanediyl groups with one or more alicyclic hydrocarbon groups, such as -alkanediyl group-alicyclic hydrocarbon group, -alicyclic hydrocarbon group-alkyl group, and -alkanediyl group-alicyclic hydrocarbon group-alkyl group.

Examples of the alkanediyl group include a straight-chain alkanediyl group such as a methylene group, an ethylene group, a propane-1,3-diyl group, a butane-1,4-diyl group, a pentane-1,5-diyl group, and a hexane-1,6-diyl group; and a branched alkanediyl group such as a propane-1,2-diyl group, a butane-1,3-diyl group, a 2-methylpropane-1,2-diyl group, a 1-methylbutane-1,4-diyl group, and a 2-methylbutane-1,4-diyl group. The terminal of the branched alkanediyl group may be a methyl group.

Examples of the halogen atom included in the aliphatic hydrocarbon group included in R⁴² include a fluorine atom, a chlorine atom, a bromine atom, and an iodine atom.

Examples of the group formed by replacing —CH₂— in the aliphatic hydrocarbon group included in R⁴² with —O— or —CO— include a hydroxy group, a carboxy group, a carbonyl group, an oxy group, an alkoxy group, an alkoxycarbonyl group, an alkylcarbonyl group, an alkylcarbonyloxy group, an alkanediyloxy group, an alkanediyloxycarbonyl group, an alkanediylcarbonyl group, an alkanediylcarbonyloxy group, a cycloalkoxy group, a cycloalkylalkoxy group, and a group that combines two or more of these groups. Examples of these replaced groups include the same groups as those exemplified in the present specification, within the acceptable range of the upper limit of the number of carbon atoms.

As the structural unit (a4), R⁴² is preferably an aliphatic hydrocarbon group having a fluorine atom, and examples of the structural unit (a4) in which R⁴² is an aliphatic hydrocarbon group having a fluorine atom include a structural unit represented by formula (a4-1) (hereinafter, may be referred to as “structural unit (a4-1)”), a structural unit represented by formula (a4-2) (hereinafter, may be referred to as “structural unit (a4-2)”), a structural unit represented by formula (a4-3) (hereinafter, may be referred to as “structural unit (a4-3)”), and a structural unit represented by formula (a4-A) (hereinafter, may be referred to as “structural unit (a4-A)”).

The structural unit represented by formula (a4-1) is a structural unit represented by the following formula.

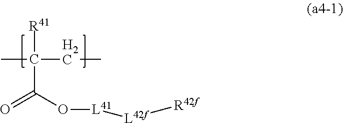

[wherein, in formula (a4-1),
R⁴¹ represents a hydrogen atom or a methyl group;
L⁴¹ represents a single bond or an alkanediyl group having 1 to 4 carbon atoms;
L^{42f} represents an alkanediyl group having 1 to 8 carbon atoms and a fluorine atom, or a cycloalkanediyl group having 3 to 12 carbon atoms and a fluorine atom;
R^{42f} represents a hydrogen atom or a fluorine atom.]

Examples of the alkanediyl groups in L⁴¹ include those exemplified for R⁴².

Examples of the alkanediyl group having a fluorine atom in L^{42f} includes: a straight-chain alkanediyl group having a fluorine atom such as a methylene group having a fluorine atom, an ethylene group having a fluorine atom, a propane-1,3-diyl group having a fluorine atom, a butane-1,4-diyl group having a fluorine atom, a pentane-1,5-diyl group having a fluorine atom, a hexane-1,6-diyl group having a fluorine atom, a heptane-1,7-diyl group having a fluorine atom, and an octane-1,8-diyl group having a fluorine atom;
a branched alkanediyl group having a fluorine atom such as an ethane-1,1-diyl group having a fluorine atom, a propane-1,1-diyl group having a fluorine atom, a propane-1,2-diyl group having a fluorine atom, a propane-2,2-diyl group having a fluorine atom, a pentane-2,4-diyl group having a fluorine atom, a 2-methylpropane-1,3-diyl group having a fluorine atom, a 2-methylpropane-1,2-diyl group having a fluorine atom, a pentane-1,4-diyl group having a fluorine atom, and a 2-methylbutane-1,4-diyl group having a fluorine atom. The terminal of the branched alkanediyl group may be a methyl group optionally having a fluorine atom.

Examples of the cycloalkanediyl group having a fluorine atom in L^{42f} include: a monocyclic cycloalkanediyl group having a fluorine atom such as a cyclobutane-1,3-diyl group having a fluorine atom, a cyclopentane-1,3-diyl group having a fluorine atom, a cyclohexane-1,4-diyl group having a fluorine atom, a cyclohexene-3,6-diyl group having a fluorine atom, a cycloheptane-1,4-diyl group having a fluorine atom, and a cyclooctane-1,5-diyl group having a fluorine atom;
a polycyclic cycloalkanediyl group having a fluorine atom such as a norbornane-1,4-diyl group having a fluorine atom, a norbornane-2,5-diyl group having a fluorine atom, a 5-norbornene-2,3-diyl group having a fluorine atom, an adamantane-1,5-diyl group having a fluorine atom, and an adamantane-2,6-diyl group having a fluorine atom.

The number of fluorine atoms in the alkanediyl group and cycloalkanediyl group having a fluorine atom in L^{42f} may be 1 or more, and is preferably 2 or more.

The alkanediyl group and cycloalkanediyl group having a fluorine atom in L^{42f} are preferably a perfluoroalkanediyl group and a perfluorocycloalkanediyl group, respectively.

Examples of the perfluoroalkanediyl group in L^{42f} include a difluoromethylene group, a perfluoroethylene group, a perfluoroethylfluoromethylene group, a perfluoropropane-1,3-diyl group, a perfluoropropane-1,2-diyl group, a perfluoropropane-2,2-diyl group, a perfluorobutane-1,4-diyl group, a perfluorobutane-2,2-diyl group, a perfluorobutane-1,2-diyl group, a perfluoropentane-1,5-diyl group, a perfluoropentane-2,2-diyl group, a perfluoropentane-3,3-diyl group, a perfluorohexane-1,6-diyl group, a perfluorohexane-2,2-diyl group, a perfluorohexane-3,3-diyl group, a perfluoroheptane-1,7-diyl group, a perfluoroheptane-2,2-diyl group, a perfluoroheptane-3,4-diyl group, a perfluoroheptane-4,4-diyl group, a perfluorooctane-1,8-diyl group, a perfluorooctane-2,2-diyl group, a perfluorooctane-3,3-diyl group, and a perfluorooctane-4,4-diyl group.

Examples of the perfluorocycloalkanediyl group in L^{42f} include a perfluorocyclohexanediyl group, a perfluorocyclopentanediyl group, a perfluorocycloheptanediyl group, and a perfluoroadamantanediyl group.

L⁴¹ is preferably a single bond or an alkanediyl group having 1 to 3 carbon atoms, more preferably a single bond, a methylene group, or an ethylene group, and further preferably a single bond or a methylene group.

L⁴² is preferably a perfluoroalkanediyl group having 1 to 8 carbon atoms or a perfluorocycloalkanediyl group having 3 to 12 carbon atoms, more preferably a perfluoroalkanediyl group having 1 to 6 carbon atoms, and further preferably a perfluoroalkanediyl group having 1 to 3 carbon atoms.

Examples of the structural unit (a4-1) include the structural units shown below and the structural units in which the methyl group corresponding to R⁴¹ in the structural unit (a4-1) in the following structural units is replaced with a hydrogen atom.

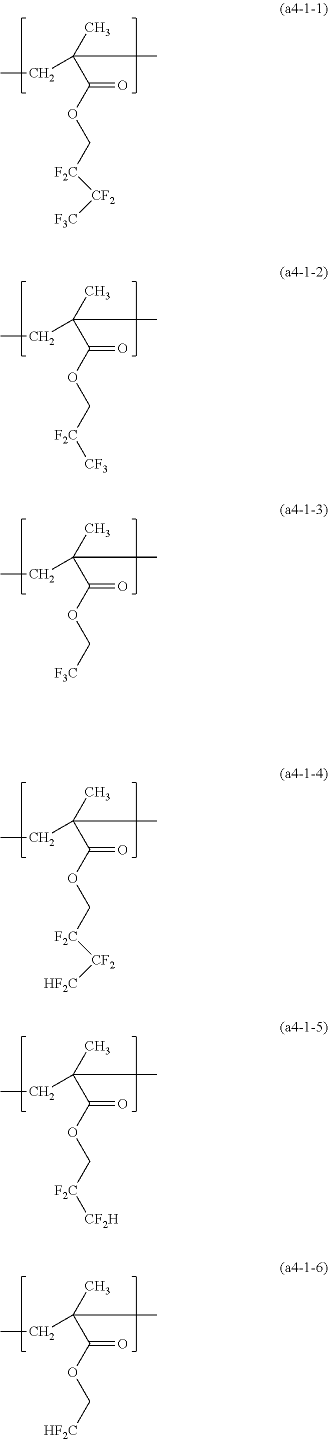

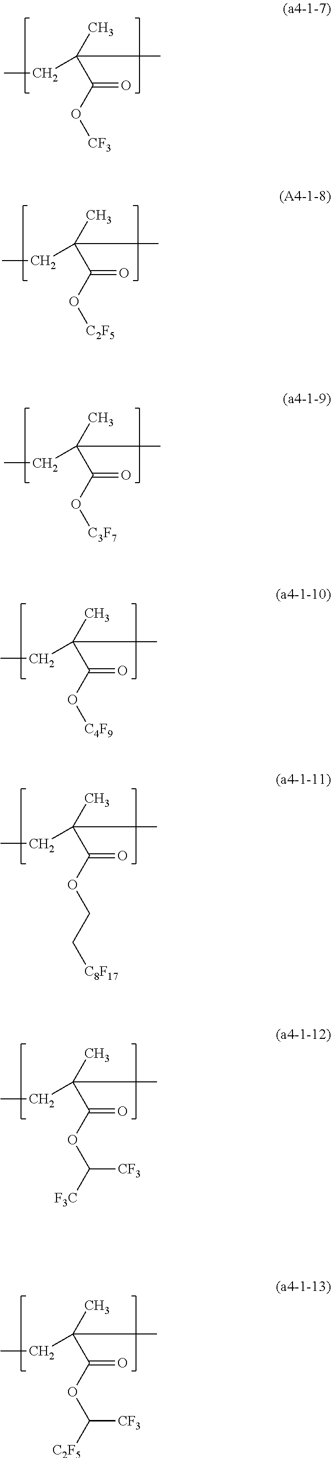

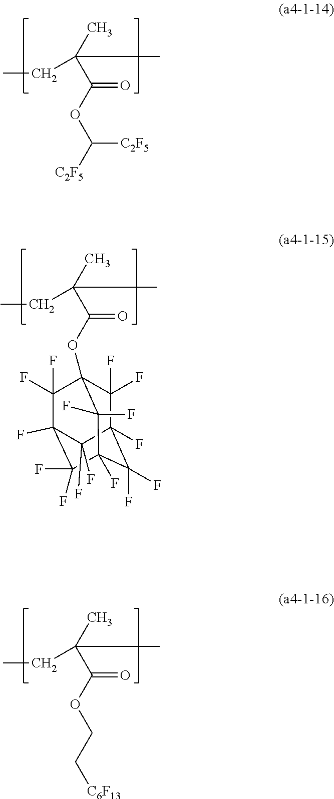

The structural unit represented by formula (a4-2) is a structural unit represented by the following formula.

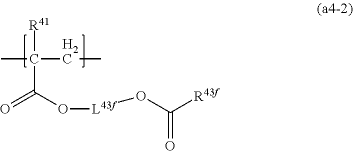

[wherein, in formula (a4-2),
R⁴¹ represents a hydrogen atom or a methyl group;
L^{43f} represents an aliphatic hydrocarbon group having 1 to 20 carbon atoms and optionally having a fluorine atom, and —CH₂— contained in the aliphatic hydrocarbon group is optionally replaced with —O— or —CO—; and
R^{43f} represents an aliphatic hydrocarbon group having 1 to 20 carbon atoms and optionally having a fluorine atom, and —CH₂— contained in the aliphatic hydrocarbon group is optionally replaced with —O— or —CO—;
provided that at least one of L^{43f} and R^{43f} has a fluorine atom, and the upper limit of the total number of carbon atoms of L^{43f} and R^{43f} is 21.]

Examples of the aliphatic hydrocarbon group in L^{43f} and R^{43f} include a straight-chain or branched chain hydrocarbon group and monocyclic or polycyclic alicyclic hydrocarbon group, as well as the group formed by combining these groups. Examples of the aliphatic hydrocarbon group in L^{43f} and R^{43f} include the same groups as the exemplified aliphatic hydrocarbon group in R⁴² in formula (a4), within the range permitted by the upper limit of the total number of carbon atoms of L^{43f} and R⁴³.

As the aliphatic hydrocarbon group of L^{43f}, an alkanediyl group having 1 to 6 carbon atoms or a group represented by the formula (L43f-1) is preferable.

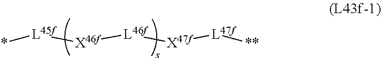

[wherein, in formula (L43f-1),
s represents 0 or 1;
L^{45f} and L^{46f} each independently represent a divalent aliphatic hydrocarbon group having 1 to 5 carbon atoms and optionally having a fluorine atom;
L^{47f} represents a single bond or a divalent aliphatic hydrocarbon group having 1 to 5 carbon atoms and optionally having a fluorine atom;
X^{46f} and X^{47f} each independently represent —O—, —CO—, —CO—O—, or —O—CO—, provided that the total number of carbon atoms in L^{45f}, L^{46f}, L^{47f}, X^{46f}, and X^{47f} is 7 or less; and
* and ** represent bonding sites, and ** represents the bonding site to —O—CO—R^{43f}.]

Examples of the divalent aliphatic hydrocarbon groups represented by L^{45f}, L^{46f}, and L^{47f} in the group represented by formula (L43f-1) include: a straight-chain or branched alkanediyl group, a monocyclic or polycyclic divalent alicyclic hydrocarbon group; and a divalent aliphatic hydrocarbon group formed by combining the alkanediyl group and the divalent alicyclic hydrocarbon group. Specific examples thereof include a methylene group, an ethylene group, a propane-1,3-diyl group, a propane-1,2-diyl group, a butane-1,4-diyl group, a 1-methylpropane-1,3-diyl group, a 2-methylpropane-1,3-diyl group, and a 2-methylpropane-1,2-diyl group.

s is preferably 0.

Examples of the group represented by formula (L43f-1) include the following groups. In the following examples of the groups, * and ** each represent a bonding site, and ** is the bonding site to —O—CO—R^{43f}.

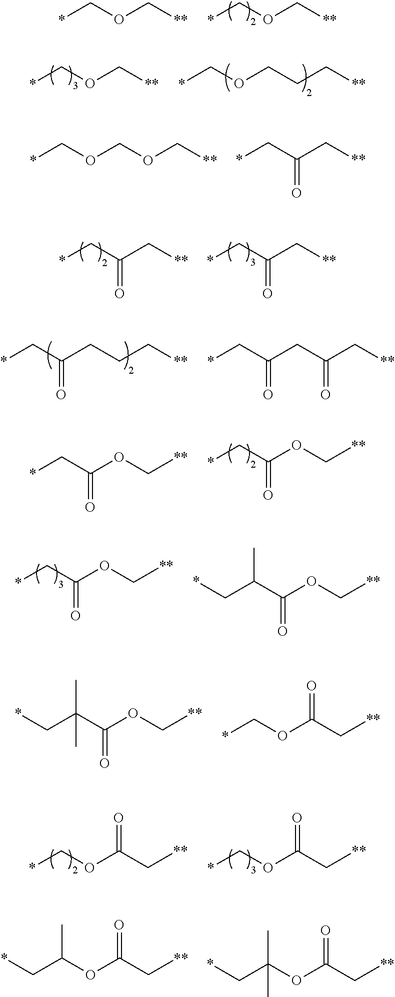

R^{43f} is preferably an aliphatic hydrocarbon group having a fluorine atom.

In this case, examples of the structural unit represented by formula (a4-2) include the structural unit represented by formula (a4-2A) or (a4-2B).

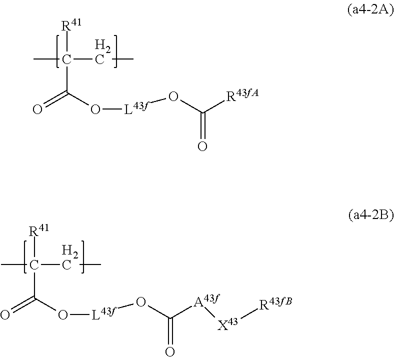

[wherein, in formula (a4-2A) and formula (a4-2B),
R⁴¹ and L^{43f} are the same as in formula (a4-2);
R^{43f} represents an aliphatic hydrocarbon group having 1 to 20 carbon atoms and optionally having a fluorine atom;
A^{43f} represents a divalent aliphatic hydrocarbon group having 1 to 17 carbon atoms and optionally having a fluorine atom, and —CH₂— contained in the aliphatic hydrocarbon group is optionally replaced with —O— or —CO—;
X⁴³ represents **—O—CO— or **—CO—O—, and ** represents the bonding site to A^{43f}; and
R^{43fB} represents an aliphatic hydrocarbon group having 1 to 17 carbon atoms and optionally having a fluorine atom,
provided that in formula (a4-2A), the total number of carbon atoms of L^{43f} and R^{43fA} is 21 or less, and in formula (a4-2B), the total number of carbon atoms of L^{43f}, A^{43f}, X⁴³, and R^{43fB} is 21 or less, and at least one of A^{43f} and R^{43B} has at least one fluorine atom.]

Examples of the aliphatic hydrocarbon group for R^{43fA} include the same aliphatic hydrocarbon group as the aliphatic hydrocarbon groups exemplified by R⁴², within the range permitted by the upper limit of the total number of carbon atoms.

R^{43fA} is preferably an alkyl group having 1 to 13 carbon atoms and a fluorine atom, a cycloalkyl group having 3 to 12 carbon atoms and a fluorine atom, or a group obtained by combining these groups, and *—(CF₂)_{n43f}—R^{42′} (wherein * represents the bonding site to the carbonyl group; n43f represents an integer of 1 to 6; R^{42′} represents a hydrogen atom or a fluorine atom) or a perfluorocycloalkyl group having 3 to 12 carbon atoms are more preferable.

Examples of the aliphatic hydrocarbon groups of A^{43f} and R^{43fB} include the same aliphatic hydrocarbon groups as those exemplified for R⁴² in formula (a4), within the range permitted by the upper limit of the total number of carbon atoms.

A^{43f} is preferably a divalent chain hydrocarbon group optionally having a fluorine atom, a divalent alicyclic hydrocarbon group, or a group formed by combining these groups, more preferably a divalent chain hydrocarbon group having a fluorine atom, and further preferably a fluorinated alkanediyl group having 1 to 6 carbon atoms.

The aliphatic hydrocarbon group optionally having a fluorine atom for R^{43fB} is preferably a chain hydrocarbon group, an alicyclic hydrocarbon group, or a combination thereof, optionally having a fluorine atom, more preferably an alkyl group having 1 to 12 carbon atoms, a cycloalkyl group having 3 to 12 carbon atoms, or a combination thereof, optionally having a fluorine atom, and further preferably a trifluoromethyl group, a difluoromethyl group, a methyl group, a perfluoroethyl group, a 2,2,2-trifluoroethyl group, a 1,1,2,2-tetrafluoroethyl group, an ethyl group, a perfluoropropyl group, a 2,2,3,3,3-pentafluoropropyl group, a propyl group, a perfluorobutyl group, a 1,1,2,2,3,3,4,4-octafluorobutyl group, a butyl group, a perfluoropentyl group, a 2,2,3,3,4,4,5,5,5-nonafluoropentyl group, a pentyl group, a hexyl group, a perfluorohexyl group, a heptyl group, a perfluoroheptyl group, an octyl group, a fluorinated alkyl group such as a perfluorooctyl group, a cyclopropylmethyl group, a cyclopropyl group, a cyclobutylmethyl group, a cyclopentyl group, a cyclohexyl group, a perfluorocyclohexyl group, an adamantyl group, an adamantylmethyl group, an adamantyldimethyl group, a norbornyl group, a norbornylmethyl group, a perfluoroadamantyl group, and a perfluoroadamantylmethyl group.

In formula (a4-2B), an example of a structure that can include the group represented by *-A^{43f}-X⁴³—R^{43fB} is the following structure (* is the bonding site with the carbonyl group).

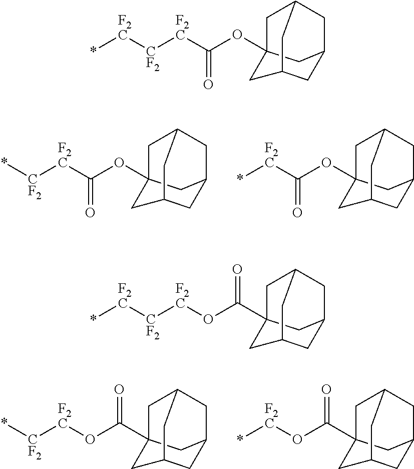

Examples of the structural unit represented by formula (a4-2A) include the structural units shown below and structural units in which the methyl group corresponding to R⁴¹ in the structural unit represented by formula (a4-2A) in the following structural units is replaced with a hydrogen atom.

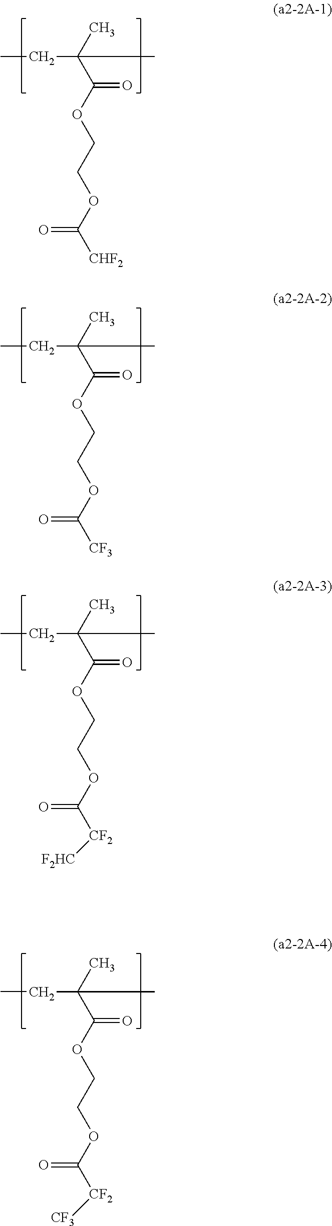

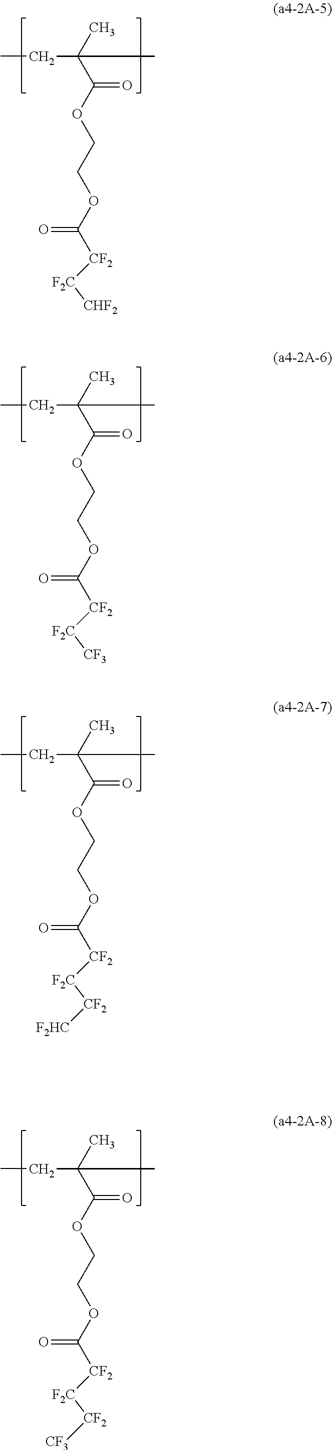

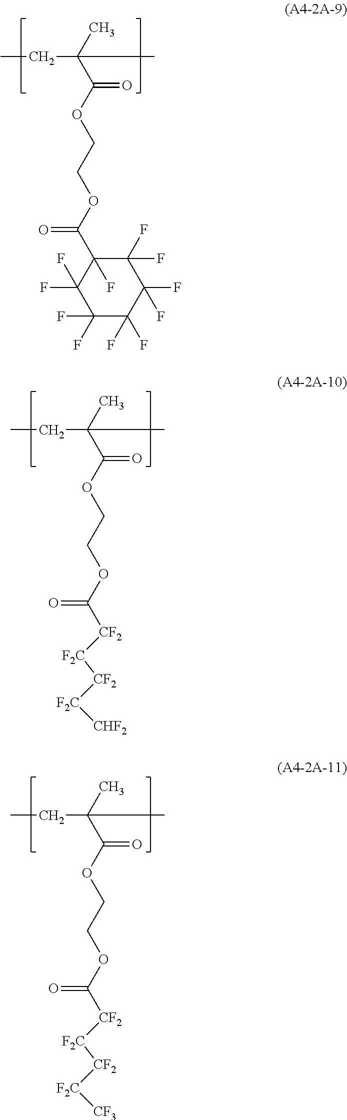

Examples of the structural unit represented by formula (a4-2B) include the structural units shown below and the structural units in which the methyl group corresponding to R⁴¹ in the structural unit represented by formula (a4-2B) in the structural units shown below is replaced with a hydrogen atom.

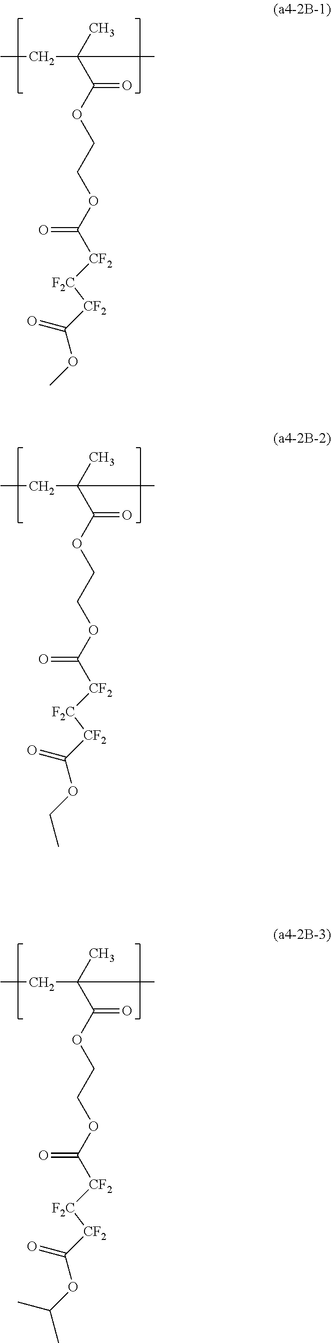

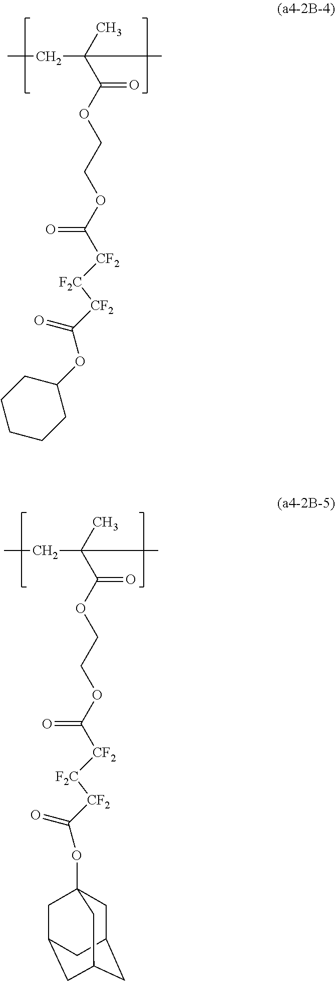

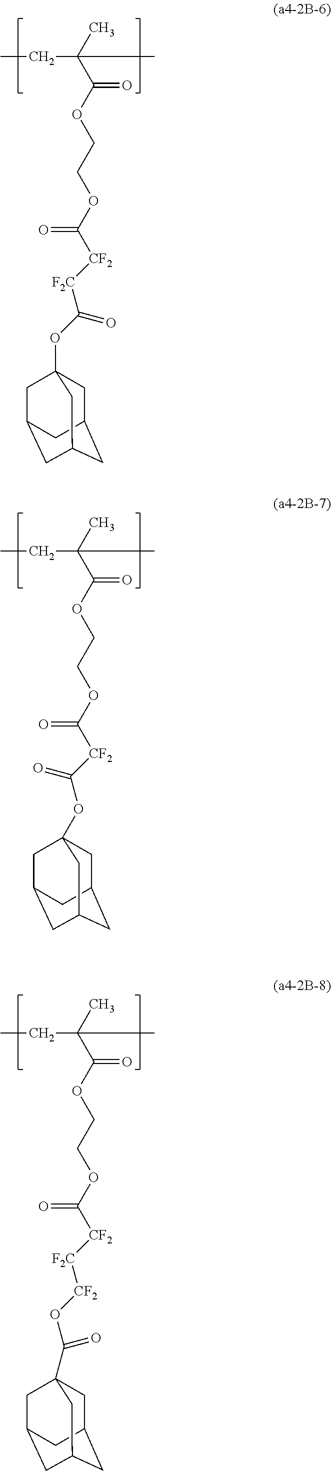

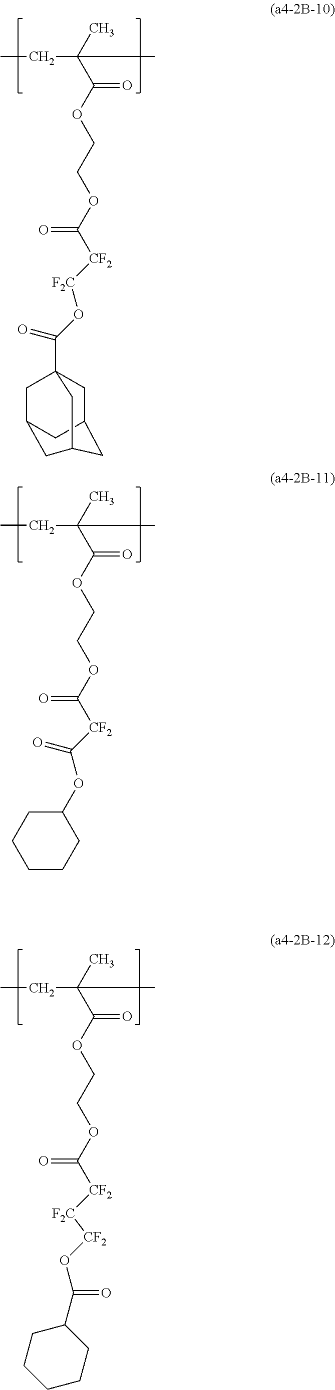

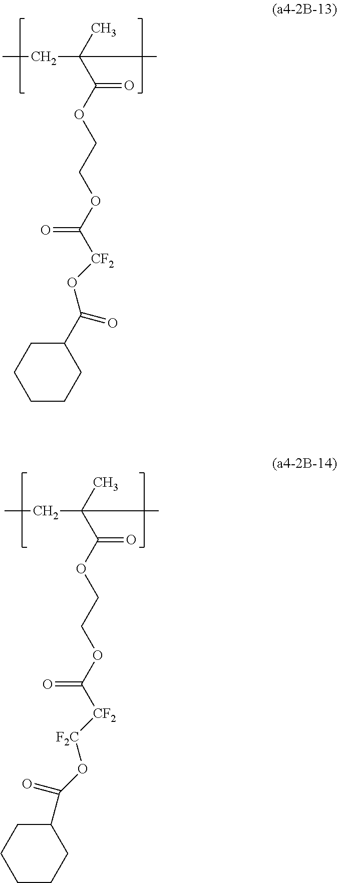

In addition, examples of the structural unit (a4) include a structural unit represented by the formula (a4-3).

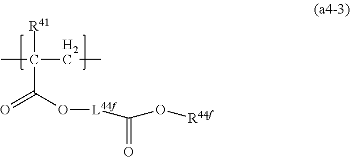

[wherein, in formula (a4-3),
R⁴¹ represents a hydrogen atom or a methyl group;
L^{44f} represents an aliphatic hydrocarbon group having 1 to 20 carbon atoms and optionally having a fluorine atom, and —CH₂— contained in the aliphatic hydrocarbon group is optionally replaced with —O— or —CO—; and
R^{44f} represents an aliphatic hydrocarbon group having 1 to 20 carbon atoms and optionally having a fluorine atom, and —CH₂— contained in the aliphatic hydrocarbon group is optionally replaced with —O— or —CO—;
provided with at least one of L^{44f} and R^{44f} has a fluorine atom, and the upper limit of the total number of carbon atoms of L^{44f} and R^{44f} is 21.]

Examples of the aliphatic hydrocarbon group in L^{44f} and R^{44f} include a straight-chain or branched chain hydrocarbon group and monocyclic or polycyclic alicyclic hydrocarbon group, as well as the group formed by combining these groups. Examples of the aliphatic hydrocarbon groups in L^{44f} and R^{44f} include the same groups as the exemplified aliphatic hydrocarbon groups for R⁴² in formula (a4), within the range permitted by the upper limit of the total number of carbon atoms of L^{44f} and R^{44f}.

L^{44f} is preferably an alkanediyl group having 1 to 14 carbon atoms (—CH₂— contained in the alkanediyl group is optionally replaced with —O— or —CO—), and more preferably a group represented by —(CH₂)ⱼ₁—, —(CH₂)ⱼ₂—O—(CH₂)ⱼ₃—, or —(CH₂)ⱼ₄—CO—O—(CH₂)ⱼ₅— (j1 to j5 each independently represent an integer of 1 to 6). In addition, also preferable is an alkanediyl group having 1 to 4 carbon atoms (one —CH₂— in the alkanediyl group is optionally replaced with —O—, and one —CH₂—CH₂— in the alkanediyl group is optionally replaced with —CO—O— or —O—CO—).

R^{44f} is preferably an aliphatic hydrocarbon group having 1 to 10 carbon atoms and having a fluorine atom, more preferably an alkyl group having 1 to 10 carbon atoms and having a fluorine atom, an alicyclic hydrocarbon group having 3 to 10 carbon atoms and having a fluorine atom, or a combined group of these groups, further preferably an alkyl group having 1 to 10 carbon atoms and having a fluorine atom, and still more preferably an alkyl group having 1 to 6 carbon atoms and having a fluorine atom.

Examples of the structural unit represented by formula (a4-3) include the following structural units and structural units represented by the following formula in which the methyl group corresponding to R⁴¹ in the structural unit (a4-3) is replaced with a hydrogen atom.

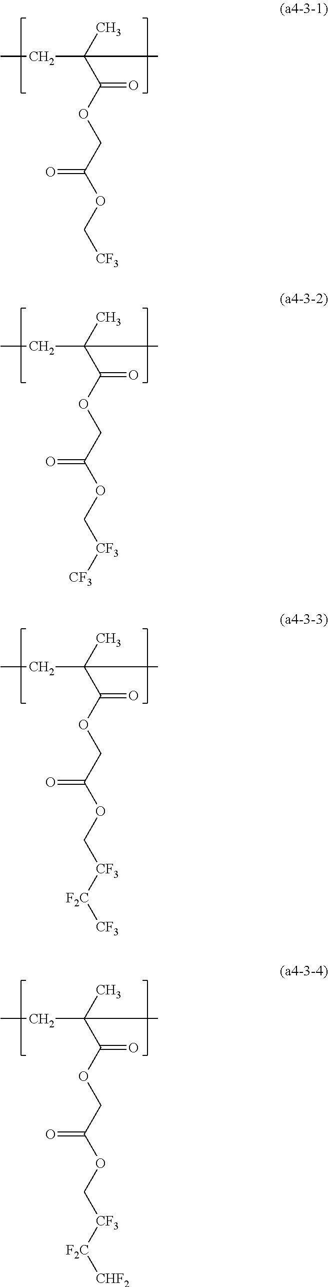

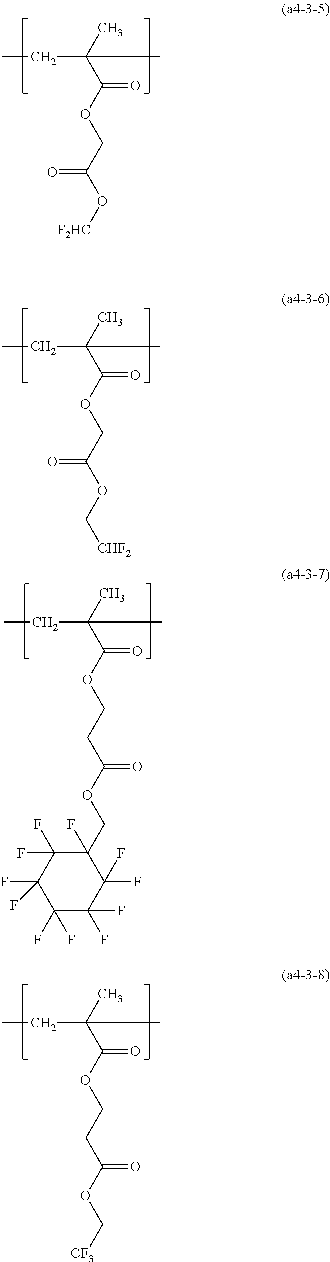

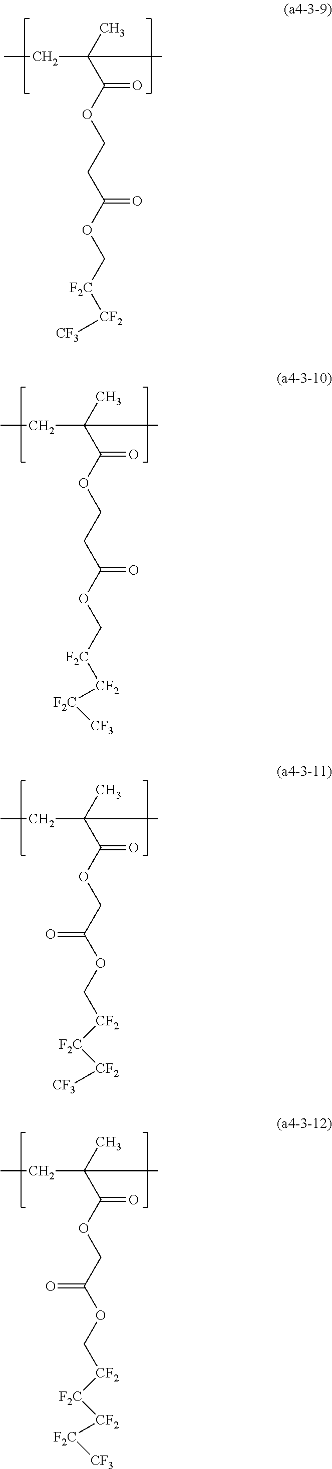

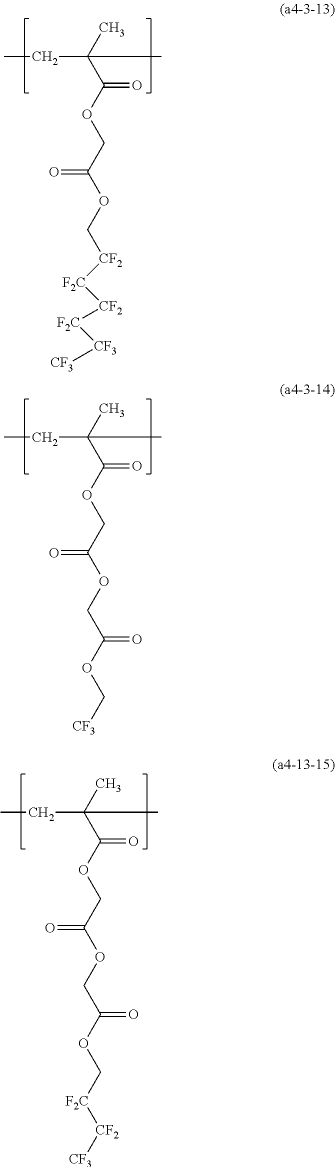

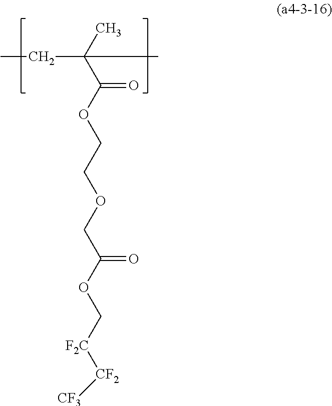

The structural unit (a4) may also be a structural unit represented by the formula (a4-A).

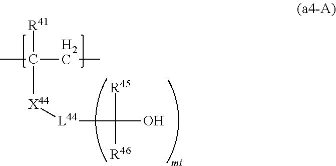

[wherein, in formula (a4-A),
R⁴⁴ represents a hydrogen atom, a halogen atom, or an alkyl group having 1 to 6 carbon atoms and optionally having a halogen atom;
X⁴⁴ represents a single bond or *—CO—O—; * represents the bonding site to the carbon atom to which R⁴⁴ is bonded;
L⁴⁴ represents an aliphatic hydrocarbon group having 1 to 28 carbon atoms and optionally having a substituent, and —CH₂— contained in the aliphatic hydrocarbon group is optionally replaced with —O—, —S—, —SO₂—, —SO—, or —CO—;
R⁴⁵ represents a fluorinated alkyl group having 1 to 8 carbon atoms;
R⁴⁶ represents a hydrogen atom or a fluorinated alkyl group having 1 to 6 carbon atoms, which may be bonded to L⁴⁴ to form an alicyclic hydrocarbon group having 3 to 12 carbon atoms, and —CH₂— contained in the alicyclic hydrocarbon group is optionally replaced with —O—; and
mi represents an integer of 1 to 4, and when mi is 2 or more, the groups in a plurality of parentheses are the same or different from each other.]

Examples of the halogen atom and alkyl group optionally having a halogen atom in R⁴⁴ include the same as those exemplified for R^{a01}, R^{a4}, and R^{a5} in formulas (a1-0) to (a1-2).

R⁴⁴ is preferably a hydrogen atom or an alkyl group having 1 to 4 carbon atoms, more preferably a hydrogen atom or an alkyl group having 1 to 3 carbon atoms, further preferably a hydrogen atom, a methyl group, or an ethyl group, and still more preferably a hydrogen atom or a methyl group.

Examples of the aliphatic hydrocarbon group for L⁴⁴ include a chain hydrocarbon group, and a monocyclic or polycyclic alicyclic hydrocarbon group (including a spiro ring, a condensed ring, a bridged ring, or the like), and may be a combined group of two or more of these groups (for example, a hydrocarbon group formed from an alicyclic hydrocarbon group and a chain hydrocarbon group).

Examples of the chain hydrocarbon group include divalent to pentavalent chain hydrocarbon groups such as an alkanediyl group, an alkanetriyl group, an alkanetetrayl group, and an alkanepentyl group.

Examples of the alkanediyl group include: a straight-chain alkanediyl group such as a methylene group, an ethylene group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; and
branched alkanediyl groups such as a ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

In addition, examples thereof include the group formed by replacing one or more of the hydrogen atoms of the above groups with a bonding site.

The number of carbon atoms in the chain hydrocarbon group is preferably 1 to 18, more preferably 1 to 12, further preferably 1 to 10, still more preferably 1 to 9, even more preferably 1 to 8, further more preferably 1 to 6, particularly preferably 1 to 5, and most preferably 1 to 4.

Examples of the monocyclic or polycyclic alicyclic hydrocarbon group include the following alicyclic hydrocarbon group. The bonding site can be set to any position.

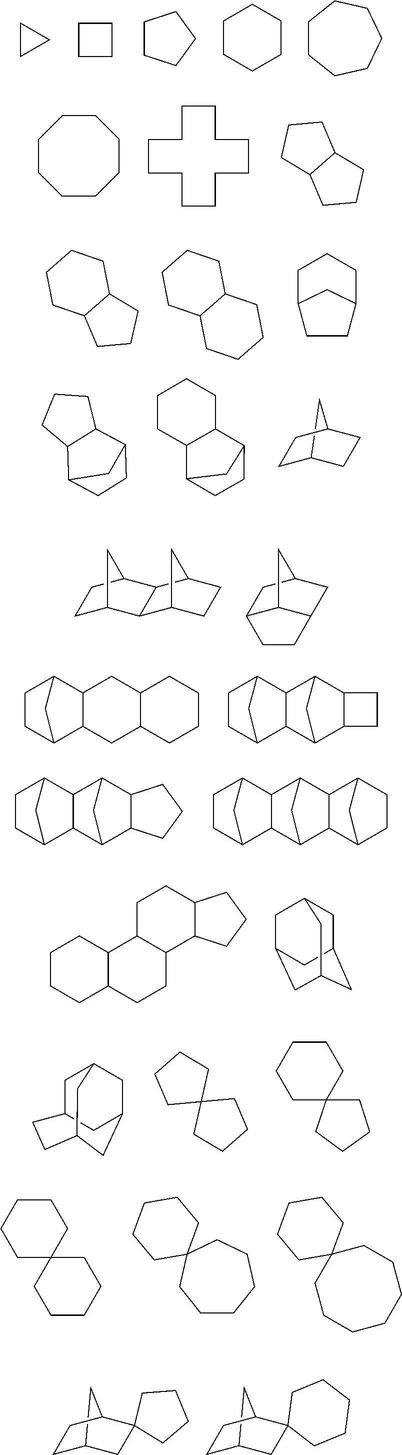

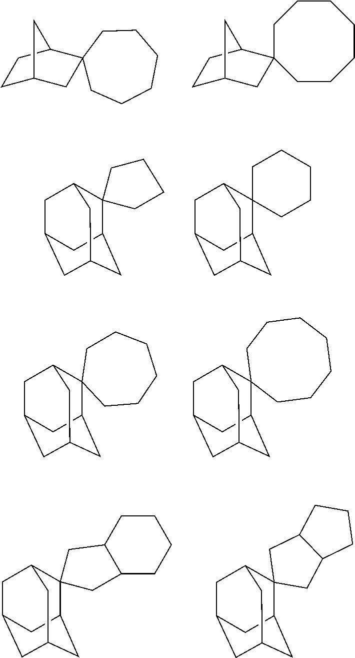

Examples of the alicyclic hydrocarbon group include divalent to pentavalent alicyclic hydrocarbon groups such as a cycloalkanediyl group, a cycloalkanetriyl group, a cycloalkanetetrayl group, and a cycloalkanepentyl group.

Specific examples thereof include: a monocyclic alicyclic hydrocarbon group such as a cyclobutane-1,3-diyl group, a cyclopentane-1,3-diyl group, a cyclohexane-1,4-diyl group, and a cyclooctane-1,5-diyl group; and
a polycyclic alicyclic hydrocarbon group such as a cycloalkanediyl group including a norbornane-1,4-diyl group, a norbornane-2,5-diyl group, an adamantane-1,5-diyl group, an adamantane-2,6-diyl group, a decahydronaphthalenediyl group, a bicyclo[3.3.0]octanediyl group, a spirocyclohexane-1,2′-cyclopentane-diyl group, a spiroadamantane-2,3′-cyclopentane-diyl group, and a spiro ring having a cycloalkanediyl group bonded to a norbornanediyl group or an adamantanediyl group via a spiro bond.

In addition, examples thereof include the group formed by replacing one or more of the hydrogen atoms of the above groups with a bonding site.

The number of carbon atoms in the alicyclic hydrocarbon group is preferably 3 to 20, more preferably 3 to 18, further preferably 3 to 16, and still more preferably 3 to 12.

Examples of the combined group of two or more include a combined group of an alicyclic hydrocarbon group and a chain hydrocarbon group. In the combination, two or more types of alicyclic hydrocarbon groups and chain hydrocarbon groups may be combined. In addition, any of the groups may be bonded to X⁴⁴.

Examples of the combined group of an alicyclic hydrocarbon group and a chain hydrocarbon group include a group in which an alicyclic hydrocarbon group is bonded to a chain hydrocarbon group (e.g., *-(chain hydrocarbon group)-(alicyclic hydrocarbon group)-), a group in which an alicyclic hydrocarbon group is bonded to a chain hydrocarbon group (e.g., *-(alicyclic hydrocarbon group)-(chain hydrocarbon group)-), a group in which an alicyclic hydrocarbon group and a chain hydrocarbon group are bonded to a chain hydrocarbon group (e.g., *-(chain hydrocarbon group)-(alicyclic hydrocarbon group)-(chain hydrocarbon group)-), a group in which an alicyclic hydrocarbon group is bonded to a chain hydrocarbon group and an alicyclic hydrocarbon group (e.g., *-(alicyclic hydrocarbon group)-(chain hydrocarbon group)-(alicyclic hydrocarbon group)-), a group in which an alicyclic hydrocarbon group, a chain hydrocarbon group, and an alicyclic hydrocarbon group are bonded to a chain hydrocarbon group (e.g., *-(chain hydrocarbon group)-(alicyclic hydrocarbon group)-(chain hydrocarbon group)-(alicyclic hydrocarbon group)-), and a group in which a chain hydrocarbon group, an alicyclic hydrocarbon group, and a chain hydrocarbon group are bonded to an alicyclic hydrocarbon group (e.g., *-(alicyclic hydrocarbon group)-(chain hydrocarbon group)-(alicyclic hydrocarbon group)-(chain hydrocarbon group)-).
* represents the bonding site to X⁴⁴.

In addition, examples thereof include the group formed by replacing one or more of the hydrogen atoms of the above groups with a bonding site.
—CH₂— contained in the aliphatic hydrocarbon group having 1 to 28 carbon atoms for L⁴⁴ is optionally replaced with —O—, —S—, —SO—, —SO₂—, or —CO—.

When —CH₂— contained in the aliphatic hydrocarbon group having 1 to 28 carbon atoms for L⁴⁴ is replaced with —O—, —S—, —SO—, —SO₂—, or —CO—, the number of carbon atoms before the replacement corresponds to the defined number of carbon atoms of the aliphatic hydrocarbon group.

Examples of the group formed by replacing —CH₂— contained in an aliphatic hydrocarbon group with —O—, —S—, —SO₂—, —SO—, or —CO— include a hydroxy group, a carboxy group, a thiol group, an alkoxy group, an alkylthio group, an alkoxycarbonyl group, an alkylsulfonyl group, an alkylcarbonyl group, an alkylcarbonyloxy group, an alkoxycarbonyloxy group, an oxy group, a carbonyl group, a thio group, a sulfonyl group, an alkanediyloxy group, an alkanediyloxycarbonyl group, an alkanediylcarbonyl group, an alkanediylcarbonyloxy group, an alkanediylsulfonyl group, an alkanediylthio group, a cycloalkoxy group, a cycloalkylalkoxy group, and a combined group of two or more of these groups. Examples of these replaced groups include the same groups as those exemplified in the present specification, within the acceptable range of the upper limit of the number of carbon atoms.

In addition, examples thereof include the group formed by replacing one or more of the hydrogen atoms of the above groups with a bonding site.

Examples of the groups in which —CH₂— in an alicyclic hydrocarbon group has been replaced with —O—, —S—, —SO—, —CO—, or —SO₂— include the same groups as those exemplified in this specification, within the range permitted by the upper limit of the number of carbon atoms.

The aliphatic hydrocarbon group for L⁴⁴ optionally has one or more substituents. Examples of such substituents include a halogen atom, a haloalkyl group having 1 to 4 carbon atoms, an alkyl group having 1 to 12 carbon atoms, an alicyclic hydrocarbon group having 3 to 18 carbon atoms, or an aromatic hydrocarbon group having 6 to 18 carbon atoms (—CH₂— contained in the alkyl group or alicyclic hydrocarbon group is optionally replaced with —O— or —CO—).

Examples of the halogen atom include a fluorine atom, a chlorine atom, a bromine atom and an iodine atom.

Examples of the haloalkyl group having 1 to 4 carbon atoms include a fluorinated alkyl group having 1 to 4 carbon atoms, a chlorinated alkyl group having 1 to 4 carbon atoms, a brominated alkyl group having 1 to 4 carbon atoms, and an iodinated alkyl group having 1 to 4 carbon atoms. Examples of the haloalkyl group include a perfluoroalkyl group having 1 to 4 carbon atoms (such as a trifluoromethyl group, a pentafluoroethyl group, a heptafluoropropyl group, and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, and an iodomethyl group. The number of carbon atoms in the haloalkyl group is preferably 1 to 3, and more preferably 1 or 2.

Examples of the alkyl group having 1 to 12 carbon atoms include a methyl group, an ethyl group, a propyl group, a butyl group, a pentyl group, a hexyl group, a heptyl group, an octyl group, a nonyl group, a decyl group, an undecyl group, and a dodecyl group. The number of carbon atoms in the alkyl group is preferably 1 to 9, more preferably 1 to 6, further preferably 1 to 4, and still more preferably 1 to 3.

Examples of the alicyclic hydrocarbon group having 3 to 18 carbon atoms include a monocyclic cycloalkyl group such as a cyclopropyl group, a cyclobutyl group, a cyclopentyl group, a cyclohexyl group, a cycloheptyl group, a cyclooctyl group, and a cyclodecyl group, and a polycyclic cycloalkyl group such as a decahydronaphthyl group, an adamantyl group, and a norbornyl group. The number of carbon atoms in the alicyclic hydrocarbon group is preferably 3 to 16, more preferably 3 to 12, and further preferably 3 to 10.

Examples of the aromatic hydrocarbon group having 6 to 18 carbon atoms include a phenyl group, a naphthyl group, a biphenyl group, an anthryl group, a phenanthryl group, and a binaphthyl group. The number of carbon atoms in the aromatic hydrocarbon group is preferably 6 to 14, more preferably 6 to 12, and further preferably 6 to 10.

When —CH₂— in the alkyl group is replaced with —O— or —CO—, the number of carbon atoms before the replacement is regarded as the total number of carbon atoms in the alkyl group. Examples of the replaced group include a hydroxy group, a carboxy group, an alkoxy group, an alkoxycarbonyl group, an alkylcarbonyl group, an alkylcarbonyloxy group, and an alkoxycarbonyloxy group. Examples of these replaced groups include the same groups as those exemplified in the present specification, within the acceptable range of the upper limit of the number of carbon atoms.

In addition, examples of the group formed by replacing —CH₂— contained in an alicyclic hydrocarbon group with —O— or —CO— include the same groups as those exemplified in this specification, within the range permitted by the upper limit of the number of carbon atoms.

The substituent that the aliphatic hydrocarbon group for L⁴⁴ optionally has is preferably a halogen atom, a haloalkyl group having 1 to 3 carbon atoms, an alkyl group having 1 to 6 carbon atoms, an alicyclic hydrocarbon group having 3 to 10 carbon atoms, or an aromatic hydrocarbon group having 6 to 10 carbon atoms (—CH₂— contained in the alkyl group or the alicyclic hydrocarbon group is optionally replaced with —O— or —CO—), more preferably a fluorine atom, an iodine atom, a perfluoroalkyl group having 1 to 3 carbon atoms, or an alkyl group having 1 to 4 carbon atoms (—CH₂— contained in the alkyl group is optionally replaced with —O— or —CO—), and further preferably a fluorine atom, an iodine atom, a trifluoromethyl group, a methyl group, a hydroxy group, or a methoxy group.

In the case of no formation of an alicyclic hydrocarbon group together with the fluorinated alkyl group of R⁴⁶, L⁴⁴ is preferably a chain hydrocarbon group having 1 to 12 carbon atoms and optionally having a substituent (—CH₂— contained in the chain hydrocarbon group is optionally replaced with —O— or —CO—), an alicyclic hydrocarbon group having 3 to 18 carbon atoms and optionally having a substituent (—CH₂— contained in the alicyclic hydrocarbon group is optionally replaced with —O— or —CO—), or a group formed by combining a chain hydrocarbon group having 1 to 6 carbon atoms and optionally having a substituent and an alicyclic hydrocarbon group having 3 to 18 carbon atoms and optionally having a substituent (—CH₂— contained in the chain hydrocarbon group is optionally replaced with —O— or —CO—, and —CH₂— contained in the alicyclic hydrocarbon group is optionally replaced with —O— or —CO—), more preferably a chain hydrocarbon group having 1 to 8 carbon atoms and optionally having a substituent (—CH₂— contained in the chain hydrocarbon group is optionally replaced with —O— or —CO—), a group formed by combining an alicyclic hydrocarbon group having 3 to 12 carbon atoms and optionally having a substituent, or a chain hydrocarbon group having 1 to 6 carbon atoms and optionally having a substituent, and an alicyclic hydrocarbon group having 3 to 18 carbon atoms and optionally having a substituent (—CH₂— contained in the chain hydrocarbon group is optionally replaced with —O— or —CO—), further preferably a chain hydrocarbon group having 1 to 8 carbon atoms and optionally having a substituent (—CH₂— contained in the chain hydrocarbon group is optionally replaced with —O— or —CO—), an alicyclic hydrocarbon group having 5 to 12 carbon atoms and optionally having a substituent, a group formed by combining a chain hydrocarbon group having 1 to 6 carbon atoms with an alicyclic hydrocarbon group having 5 to 12 carbon atoms and optionally having a substituent (—CH₂— contained in the chain hydrocarbon group is optionally replaced with —O— or —CO—), or a group formed by combining an alicyclic hydrocarbon group having 5 to 12 carbon atoms and optionally having a substituent, a chain hydrocarbon group having 1 to 6 carbon atoms, and an alicyclic hydrocarbon group having 5 to 12 carbon atoms and optionally having a substituent (—CH₂— contained in the chain hydrocarbon group is optionally replaced with —O— or —CO—).

Examples of the fluorinated alkyl group having 1 to 8 carbon atoms in R⁴⁵ include a difluoromethyl group, a trifluoromethyl group, a 1,1-difluoroethyl group, a 2,2-difluoroethyl group, a 2,2,2-trifluoroethyl group, a perfluoroethyl group, a 1,1,2,2-tetrafluoropropyl group, a 1,1,2,2,3,3-hexafluoropropyl group, a perfluoroethylmethyl group, a 1-(trifluoromethyl)-1,2,2,2-tetrafluoroethyl group, a perfluoropropyl group, a 1,1,2,2-tetrafluorobutyl group, a 1,1,2,2,3,3-hexafluorobutyl group, a 1,1,2,2,3,3,4,4-octafluorobutyl group, a perfluorobutyl group, a 1,1-bis(trifluoro)methyl-2,2,2-trifluoroethyl group, a 2-(perfluoropropyl)ethyl group, a 1,1,2,2,3,3,4,4-octafluoropentyl group, a perfluoropentyl group, a 1,1,2,2,3,3,4,4,5,5-decafluoropentyl group, a 1,1-bis(trifluoromethyl)-2,2,3,3,3-pentafluoropropyl group, a perfluoropentyl group, a 2-(perfluorobutyl)ethyl group, a 1,1,2,2,3,3,4,4,5,5-decafluorohexyl group, a 1,1,2,2,3,3,4,4,5,5,6,6-dodecafluorohexyl group, a perfluoropentylmethyl group, a perfluorohexyl group, a perfluoroheptyl group, and a perfluorooctyl group. The number of carbon atoms in the fluorinated alkyl group is preferably 1 to 6, more preferably 1 to 5, further preferably 1 to 4, and still more preferably 1 to 3.

Within the range permitted by the upper limit of the number of carbon atoms, examples of the fluorinated alkyl group having 1 to 6 carbon atoms in R⁴⁶ include the same fluorinated alkyl groups having 1 to 6 carbon atoms as those exemplified in the fluorinated alkyl group in R⁴⁵.

When R⁴⁶ is a fluorinated alkyl group having 1 to 6 carbon atoms, R⁴⁵ is preferably a fluorinated alkyl group having 1 to 6 carbon atoms, more preferably a fluorinated alkyl group having 1 to 3 carbon atoms, further preferably a trifluoromethyl group, a perfluoroethyl group, or a perfluoropropyl group, and still more preferably a trifluoromethyl group.

When R⁴⁶ is a hydrogen atom, R⁴⁵ is preferably a fluorinated alkyl group having 1 to 6 carbon atoms, more preferably a fluorinated alkyl group having 1 to 5 carbon atoms, and further preferably a fluorinated alkyl group having 3 to 5 carbon atoms.

When R⁴⁶ bonds with L⁴⁴ to form an alicyclic hydrocarbon group having 3 to 12 carbon atoms, examples of the group formed when R⁴⁶ bonds with L⁴⁴ include groups represented by the following formulas. In the following formulas, * and ** represent bonding sites, one of the two ** represents a bonding site with a hydroxy group and the other represents a bonding site with R⁴⁵, and L^{44A} is a part of the hydrocarbon group of L⁴⁴ and represents a single bond or an alkanediyl group having 1 to 4 carbon atoms. The number of carbon atoms in the alicyclic hydrocarbon group formed when R⁴⁶ bonds with L⁴⁴ is preferably 3 to 10, more preferably 3 to 8, and further preferably 3 to 6.

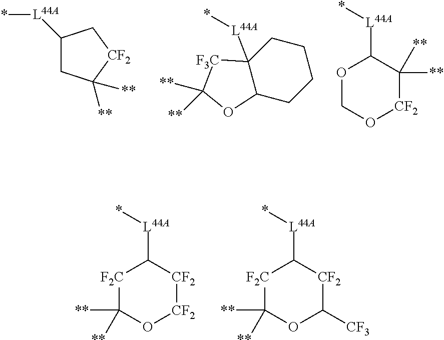

R⁴⁶ is preferably a hydrogen atom or a fluorinated alkyl group having 1 to 3 carbon atoms.

mi is preferably an integer of 1 to 3, more preferably 1 or 2, and further preferably 1.

Examples of the structural unit (a4-A) include the following structural units. Specific examples of the structural unit (a4-A) include structural units in which the methyl group corresponding to R⁴⁴ in the following structural units is replaced with a hydrogen atom, a halogen atom, a haloalkyl group, or an alkyl group other than a methyl group, and structural units in which the hydrogen atom corresponding to R⁴⁴ is replaced with a halogen atom, a haloalkyl group, or an alkyl group.

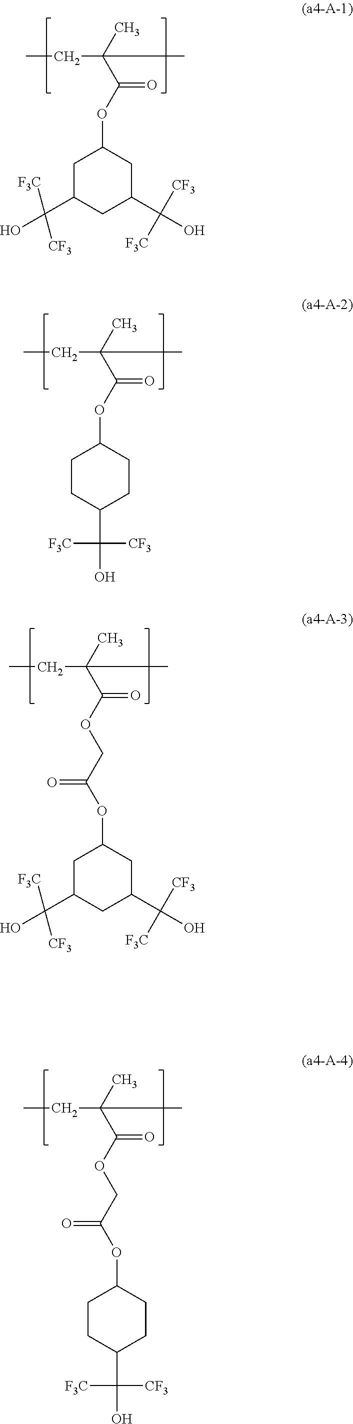

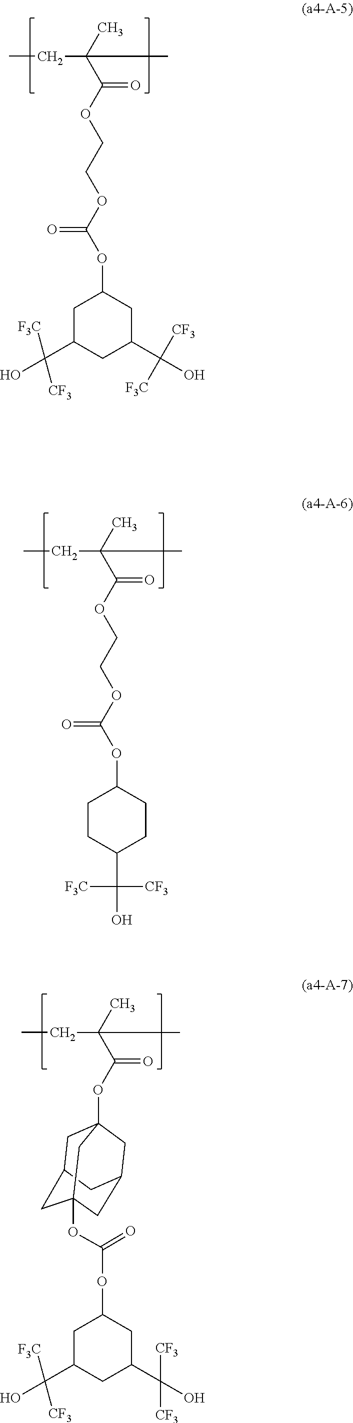

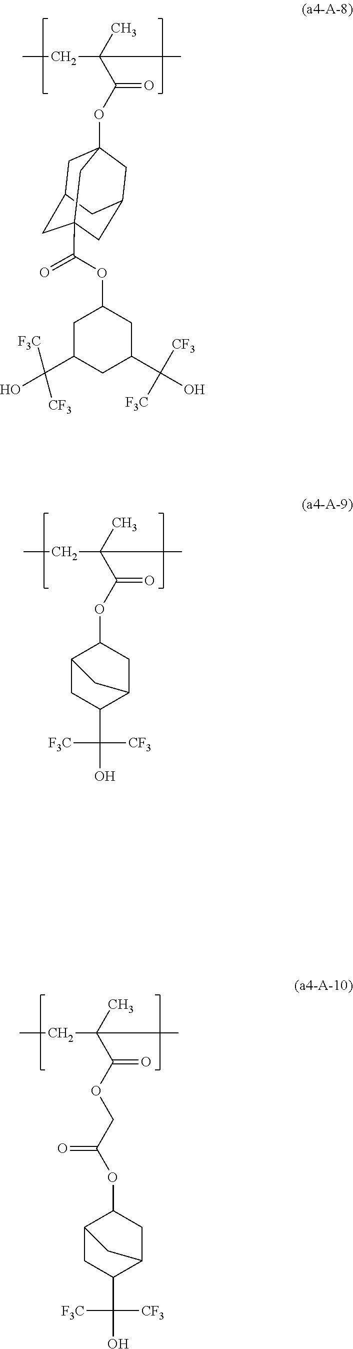

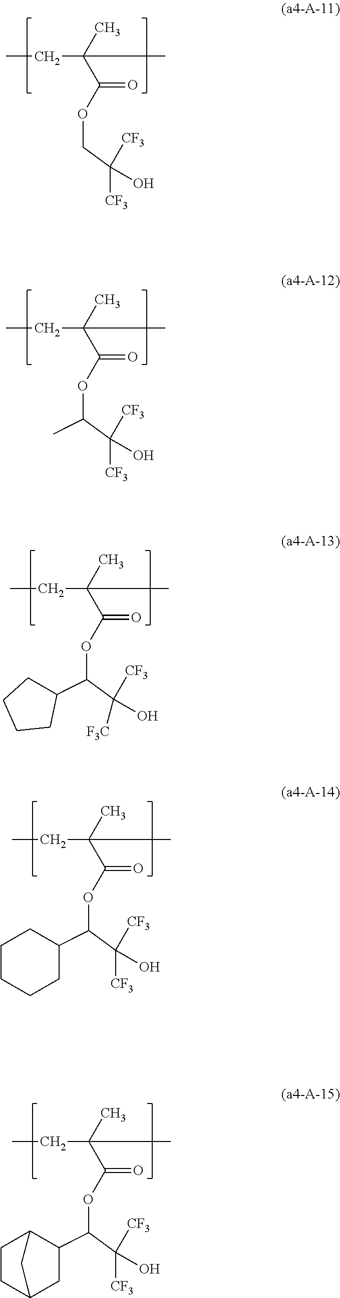

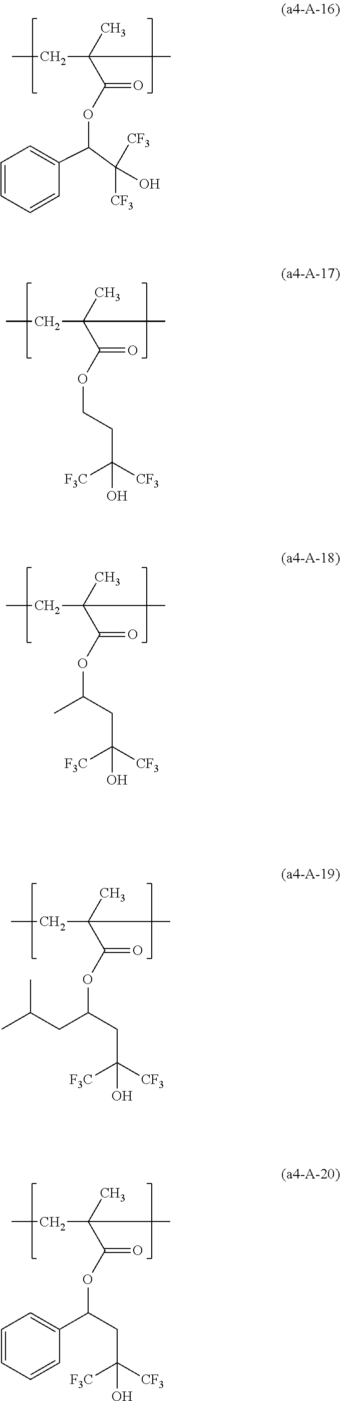

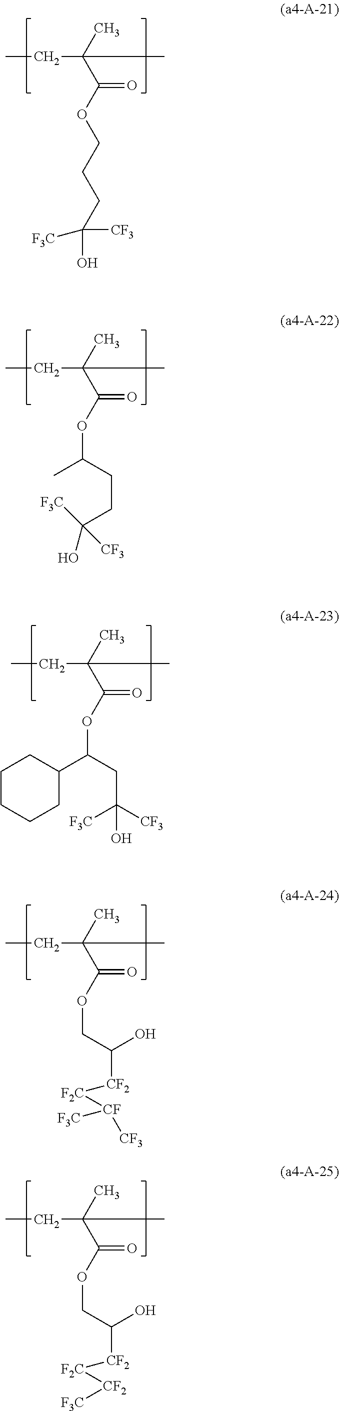

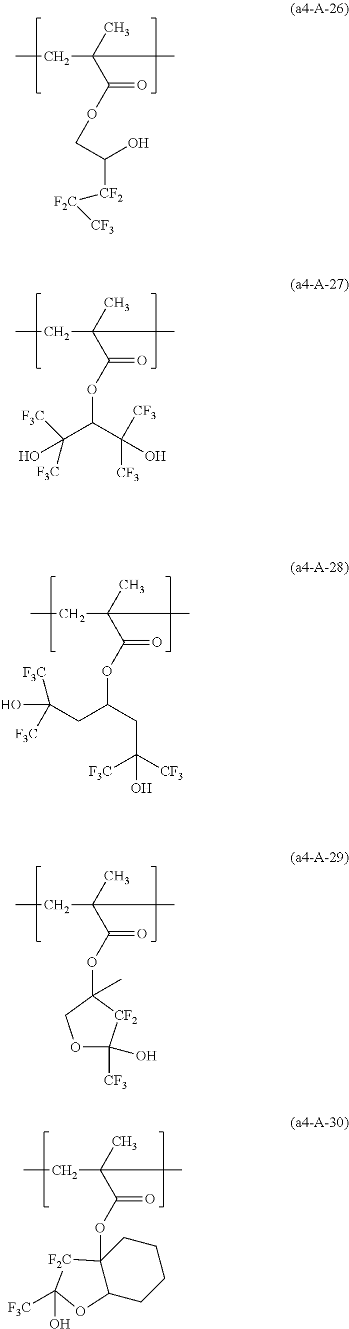

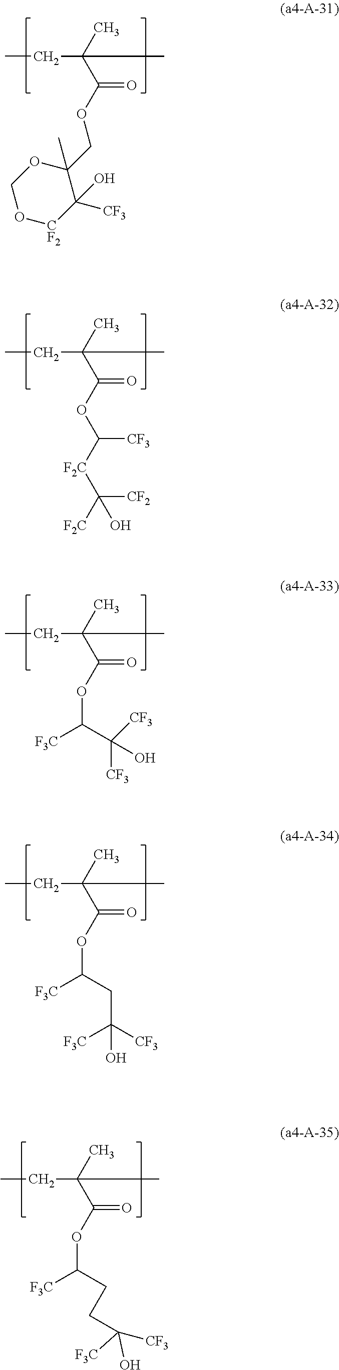

When the resin (A) etc. includes a structural unit (a4), the content ratio thereof is preferably 1 to 20 mol %, more preferably 2 to 15 mol %, and further preferably 3 to 10 mol %, with respect to the total structural units of the resin (A) etc.

<Structural Unit (a5)>

The non-eliminating hydrocarbon group included in the structural unit (a5) may be a group having a straight-chain, branched, or cyclic hydrocarbon group. Among these, the structural unit (a5) is preferably a group having an alicyclic hydrocarbon group.

Examples of the structural unit (a5) include a structural unit represented by the formula (a5-1).

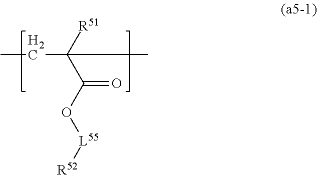

[wherein, in formula (a5-1),
R⁵¹ represents a hydrogen atom or a methyl group;
R⁵² represents an alicyclic hydrocarbon group having 3 to 18 carbon atoms, and the hydrogen atom included in the alicyclic hydrocarbon group is optionally replaced with an aliphatic hydrocarbon group having 1 to 8 carbon atoms; and
L⁵⁵ represents a single bond or a divalent saturated hydrocarbon group having 1 to 18 carbon atoms, and —CH₂— contained in the saturated hydrocarbon group is optionally replaced with —O— or —CO—.]

The alicyclic hydrocarbon group in R⁵² may be either monocyclic or polycyclic. Examples of the monocyclic alicyclic hydrocarbon group include a cyclopropyl group, a cyclobutyl group, a cyclopentyl group, and a cyclohexyl group. Examples of the polycyclic alicyclic hydrocarbon group include an adamantyl group and a norbornyl group.

Examples of the aliphatic hydrocarbon group having 1 to 8 carbon atoms include an alkyl group such as a methyl group, an ethyl group, a propyl group, an isopropyl group, a butyl group, a sec-butyl group, a tert-butyl group, a pentyl group, a hexyl group, an octyl group, and a 2-ethylhexyl group.

Examples of the alicyclic hydrocarbon group having a substituent include a 3-methyladamantyl group.

R⁵² is preferably an unsubstituted alicyclic hydrocarbon group having 3 to 18 carbon atoms, more preferably an adamantyl group, a norbornyl group, or a cyclohexyl group.

Examples of the divalent saturated hydrocarbon group in L⁵⁵ include a divalent chain saturated hydrocarbon group and a divalent alicyclic saturated hydrocarbon group, and preferably a divalent chain saturated hydrocarbon group.

Examples of the divalent chain saturated hydrocarbon group include an alkanediyl group such as a methylene group, an ethylene group, a propanediyl group, a butanediyl group, and a pentanediyl group.

The divalent alicyclic saturated hydrocarbon group may be either monocyclic or polycyclic. Examples of the monocyclic alicyclic saturated hydrocarbon group include a cycloalkanediyl group such as a cyclopentanediyl group and a cyclohexanediyl group. Examples of the polycyclic divalent alicyclic saturated hydrocarbon group include an adamantanediyl group and a norbornanediyl group.

Examples of the divalent saturated hydrocarbon group represented by L⁵⁵ in which —CH₂— is replaced with —O— or —CO— include groups represented by formulas (L1-1) to (L1-4). In the following formulas, * and ** each represent a bonding site, and * represents a bonding site with an oxygen atom.

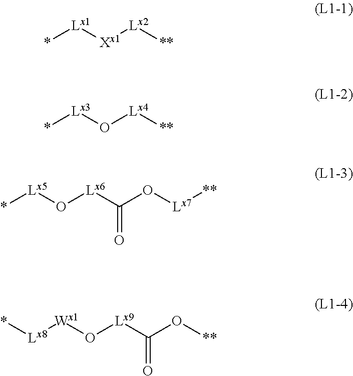

[wherein in formula (L1-1),
X^{x1} represents *—O—CO— or *—CO—O— (* represents the bonding site with L^{x1});
L^{x1} represents a divalent aliphatic saturated hydrocarbon group having 1 to 16 carbon atoms; and
L^{x2} represents a single bond or a divalent aliphatic saturated hydrocarbon group having 1 to 15 carbon atoms,
provided that the total carbon number of L^{x1} and L^{x2} is 16 or less;
in formula (L1-2),
L^{x3} represents a divalent aliphatic saturated hydrocarbon group having 1 to 17 carbon atoms; and
L^{x4} represents a single bond or a divalent aliphatic saturated hydrocarbon group having 1 to 16 carbon atoms,
provided that the total number of carbon atoms in L^{x3} and L^{x4} is 17 or less; in formula (L1-3),
L^{x5} represents a divalent aliphatic saturated hydrocarbon group having 1 to 15 carbon atoms; and
L^{x6} and L^{x7} each independently represent a single bond or a divalent aliphatic saturated hydrocarbon group having 1 to 14 carbon atoms,
provided that the total number of carbon atoms of L^{x5}, L^{x6}, and L^{x7} is 15 or less; and in formula (L1-4),
L^{x8} and L^{x9} represent a single bond or a divalent aliphatic saturated hydrocarbon group having 1 to 12 carbon atoms; and
W^{x1} represents a divalent alicyclic saturated hydrocarbon group having 3 to 15 carbon atoms,
provided that the total number of carbon atoms of L^{x8}, L^{x9}, and W^{x1} is 15 or less.]

L^{x1} is preferably a divalent aliphatic saturated hydrocarbon group having 1 to 8 carbon atoms, more preferably a methylene group, or an ethylene group.

L^{x2} is preferably a single bond or a divalent aliphatic saturated hydrocarbon group having 1 to 8 carbon atoms, more preferably a single bond.

L^{x3} is preferably a divalent aliphatic saturated hydrocarbon group having 1 to 8 carbon atoms.

L^{x4} is preferably a single bond or a divalent aliphatic saturated hydrocarbon group having 1 to 8 carbon atoms.

L^{x5} is preferably a divalent aliphatic saturated hydrocarbon group having 1 to 8 carbon atoms, more preferably a methylene group or an ethylene group.

L^{x6} is preferably a single bond or a divalent aliphatic saturated hydrocarbon group having 1 to 8 carbon atoms, more preferably a methylene group or an ethylene group.

L^{x7} is preferably a single bond or a divalent aliphatic saturated hydrocarbon group having 1 to 8 carbon atoms.

L^{x8} is preferably a single bond or a divalent aliphatic saturated hydrocarbon group having 1 to 8 carbon atoms, more preferably a single bond or a methylene group.

L^{x9} is preferably a single bond or a divalent aliphatic saturated hydrocarbon group having 1 to 8 carbon atoms, more preferably a single bond or a methylene group.

W^{x1} is preferably a divalent alicyclic saturated hydrocarbon group having 3 to 10 carbon atoms, more preferably a cyclohexanediyl group or an adamantanediyl group.

Examples of the group represented by formula (L1-1) include the divalent groups shown below.

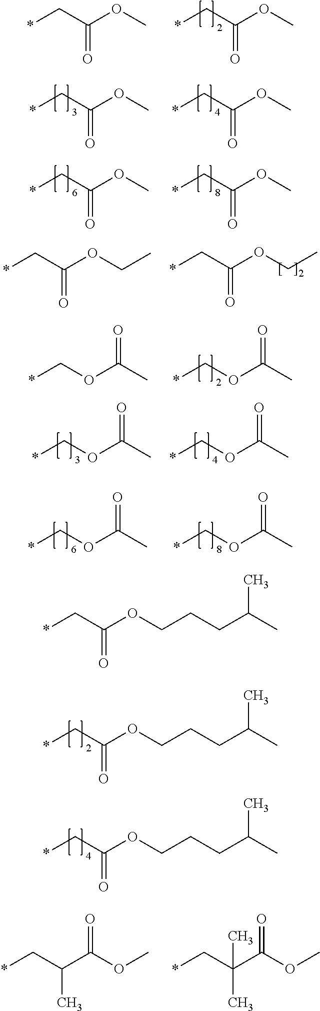

Examples of the group represented by formula (L1-2) include the divalent groups shown below.

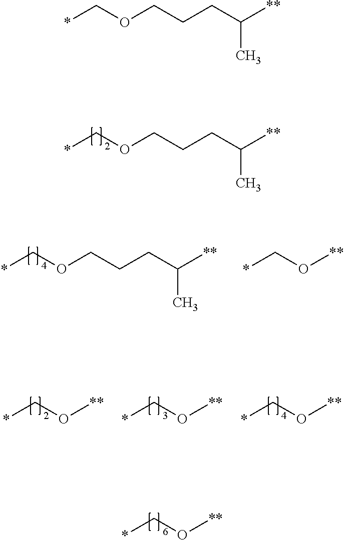

Examples of the group represented by formula (L1-3) include the divalent groups shown below.

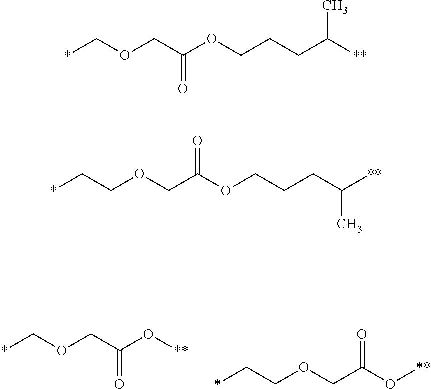

Examples of the group represented by formula (L1-4) include the divalent groups shown below.

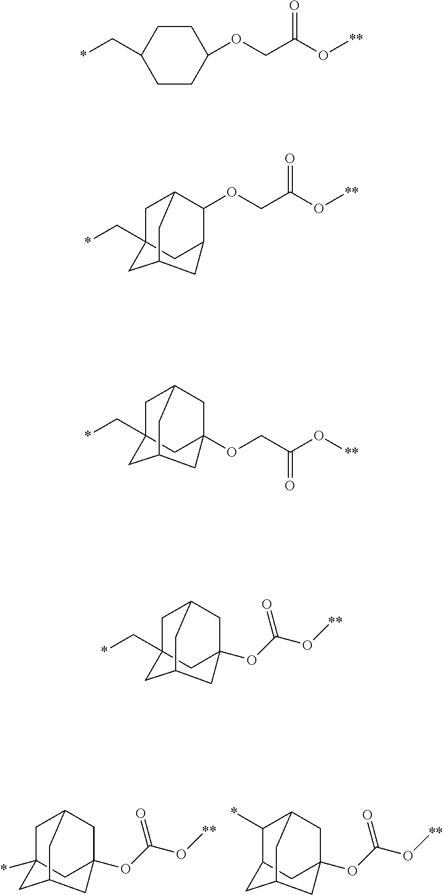

L⁵⁵ is preferably a single bond or a group represented by formula (L1-1).

Examples of the structural unit (a5-1) include the structural units shown below and the structural units in which the methyl group corresponding to R⁵¹ in the structural unit (a5-1) shown below is replaced with a hydrogen atom.

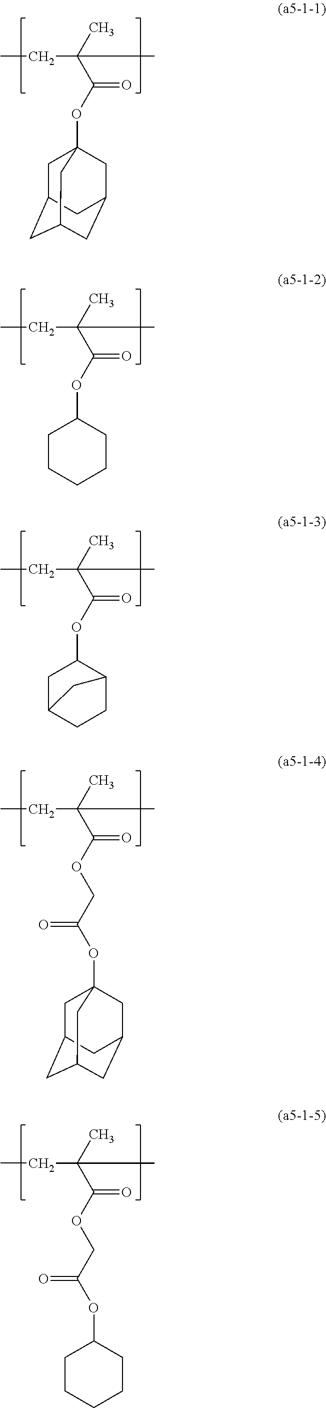

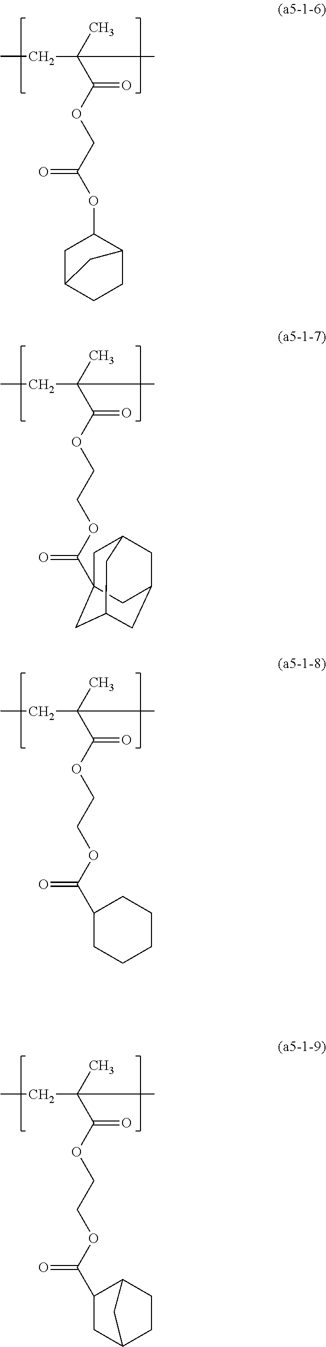

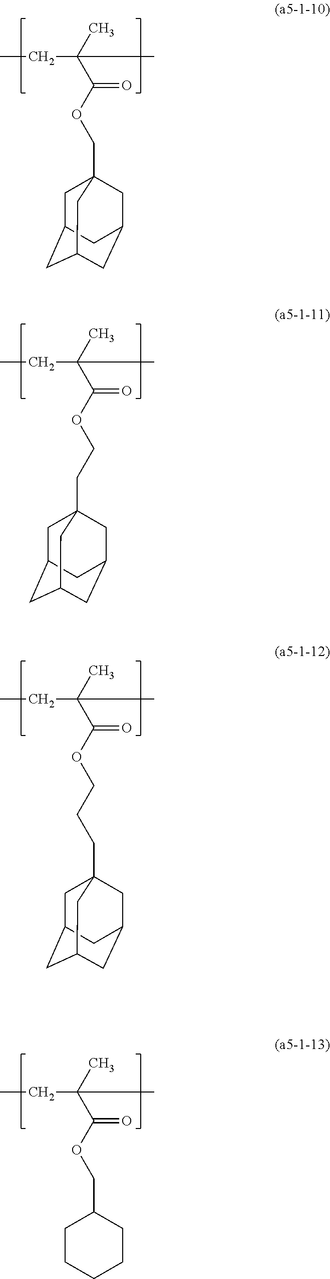

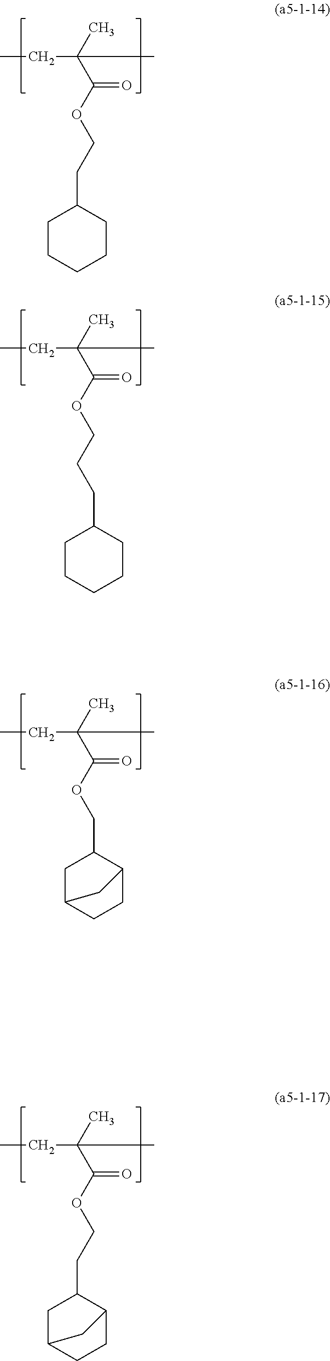

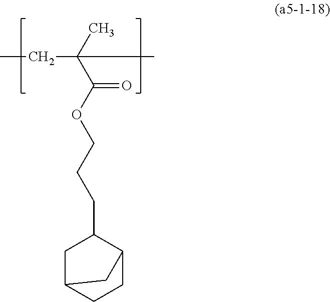

When the resin (A) etc. includes a structural unit (a5), the content ratio thereof is preferably 1 to 30 mol %, more preferably 2 to 20 mol %, and further preferably 3 to 15 mol %, with respect to the total structural units of the resin (A) etc.

<Structural Unit (a6)>

A structural unit (a6) is a structural unit having a —SO₂— group, and preferably has a —SO₂— group in the side chain.

The structural unit having a —SO₂— group optionally has a straight-chain structure having a —SO₂— group, optionally has a branched structure having a —SO₂— group, or optionally has a cyclic structure (monocyclic and polycyclic structures) having a —SO₂— group. Preferably, the above structure unit is a structural unit having a cyclic structure having a —SO₂— group, and more preferably, a structural unit having a cyclic structure (sultone ring) including —SO₂—O—.

Examples of the sultone ring include rings represented by the following formulas (T¹-1), (T¹-2), (T¹-3), and (T¹-4). The bonding site can be set to any position. The sultone ring may be monocyclic, but is preferably polycyclic. The polycyclic sultone ring means a bridged ring including —SO₂—O— as an atomic group constituting the ring, and examples thereof include the rings represented by the formulas (T¹-1) and (T¹-2). The sultone ring, like the ring represented by formula (T¹-2), may further include a heteroatom in addition to —SO₂—O— as an atomic group constituting the ring. Examples of the heteroatom include an oxygen atom, a sulfur atom, or a nitrogen atom, and an oxygen atom is preferable.

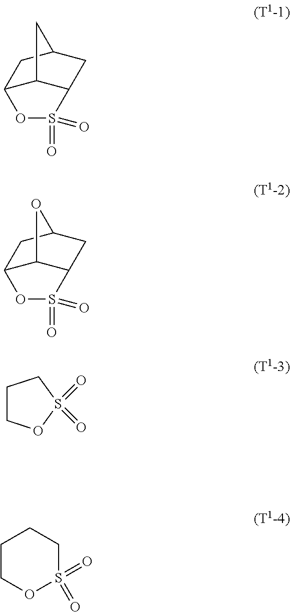

The sultone ring optionally has a substituent, and examples of the substituent include an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, and an alkylcarbonyl group having 2 to 4 carbon atoms. Examples of these groups include the same groups as those exemplified in this specification, within the range permitted by the upper limit of the number of carbon atoms.

Examples of the aryl group include a phenyl group, a naphthyl group, an anthryl group, a p-methylphenyl group, a p-tert-butylphenyl group, a p-adamantylphenyl group, a tolyl group, a xylyl group, a cumyl group, a mesityl group, a biphenyl group, a phenanthryl group, a 2,6-diethylphenyl group, and a 2-methyl-6-ethylphenyl group.

Examples of the aralkyl group include a benzyl group, a phenethyl group, a phenylpropyl group, a naphthylmethyl group, and a naphthylethyl group.

From the viewpoint of ease of production of monomers from which the structural unit (a6) is derived, a sultone ring having no substituent is preferable.

As the sultone ring, the ring represented by the following formula (T1′) is preferable.

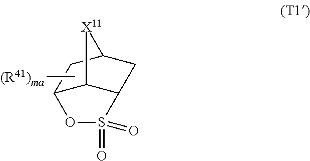

wherein in formula (T1′),
X¹¹ represents an oxygen atom, a sulfur atom, or a methylene group;
R⁴¹ represents an alkyl group having 1 to 12 carbon atoms and optionally having a halogen atom or a hydroxy group, a halogen atom, a hydroxy group, a cyano group, an alkoxy group having 1 to 12 carbon atoms, an aryl group having 6 to 12 carbon atoms, an aralkyl group having 7 to 12 carbon atoms, a glycidyloxy group, an alkoxycarbonyl group having 2 to 12 carbon atoms, or an alkylcarbonyl group having 2 to 4 carbon atoms;
ma represents an integer of 0 to 9, and when ma is 2 or more, a plurality of R⁴¹ may be the same or different from each other; and
the bonding site may be at any position.

X¹¹ is preferably an oxygen atom or a methylene group, more preferably a methylene group.

R⁴¹ may be the same as the substituent of the sultone ring, and is preferably an alkyl group having 1 to 12 carbon atoms and optionally having a halogen atom or a hydroxy group.

ma is preferably 0 or 1, more preferably 0.

Examples of the ring represented by formula (T1′) include the following rings.

The bonding site may be any position. Among these, the bonding site is preferably the 1st or 3rd position.

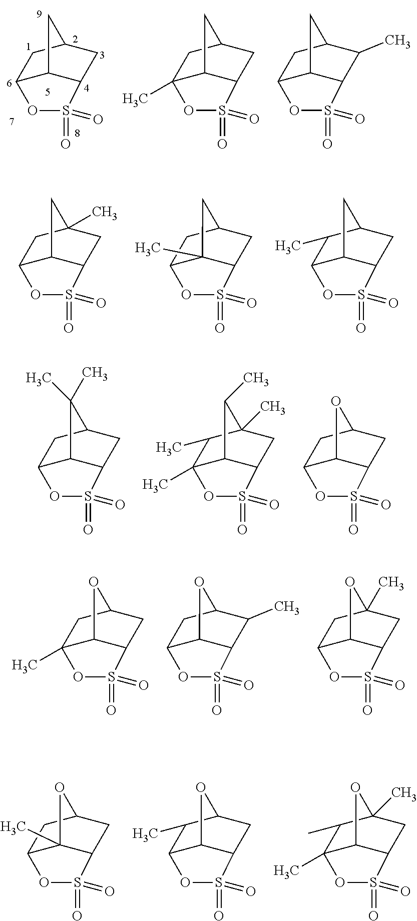

It is preferable that the structural unit having an —SO₂— group further has a group derived from a polymerizable group. Examples of the polymerizable group include a vinyl group, an acryloyl group, a methacryloyl group, an acryloyloxy group, a methacryloyloxy group, an acryloylamino group, a methacryloylamino group, an acryloylthio group, and a methacryloylthio group.

Among these, the monomer from which the structural unit (a6) is derived is preferably a monomer having an ethylenically unsaturated bond, and more preferably a (meth)acrylic monomer.

The structural unit (a6) is preferably a structural unit represented by the formula (a6-0).

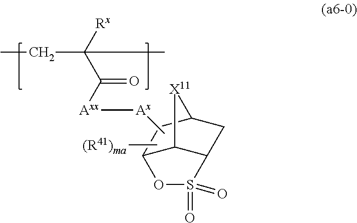

wherein in formula (a6-0),
R^{x} represents an alkyl group having 1 to 6 carbon atoms and optionally having a halogen atom, a hydrogen atom, or a halogen atom;
A^{xx} represents an oxygen atom, —N(R^{c})—, or a sulfur atom;
A^{x} represents a single bond or a divalent saturated hydrocarbon group having 1 to 18 carbon atoms, and —CH₂— contained in the saturated hydrocarbon group is optionally replaced with —O—, —CO—, or —N(R^{d})—;
X¹¹ represents an oxygen atom, a sulfur atom, or a methylene group;
R⁴¹ has the same meaning as above;
ma represents an integer of 0 to 9, and when ma is 2 or more, a plurality of R⁴¹ may be the same or different from each other; and
R^{c} and R^{d} each independently represent a hydrogen atom or an alkyl group having 1 to 6 carbon atoms.

Examples of R^{x}, R^{c}, and R^{d} include the same as those exemplified for R^{a01}, R^{a4}, and R^{a5} in formula (a1-0) to formula (a1-2).

R^{x} is preferably a hydrogen atom or an alkyl group having 1 to 4 carbon atoms, more preferably a hydrogen atom, a methyl group, or an ethyl group, and further preferably a hydrogen atom or a methyl group.

Examples of the divalent saturated hydrocarbon group for A^{x} include a straight-chain alkanediyl group, a branched alkanediyl group, and a monocyclic or polycyclic divalent alicyclic saturated hydrocarbon group, and the above group may be a combination of two or more of these groups.

Specific examples thereof include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, a heptadecane-1,17-diyl group, an ethane-1,1-diyl group, a propane-1,1-diyl group, and a propane-2,2-diyl group;
a branched alkanediyl group such as a butane-1,3-diyl group, a 2-methylpropane-1,3-diyl group, a 2-methylpropane-1,2-diyl group, a pentane-1,4-diyl group, and a 2-methylbutane-1,4-diyl group;
a monocyclic divalent alicyclic saturated hydrocarbon group, being a cycloalkanediyl group, such as a cyclobutane-1,3-diyl group, a cyclopentane-1,3-diyl group, a cyclohexane-1,4-diyl group, or a cyclooctane-1,5-diyl group; and
a polycyclic divalent alicyclic saturated hydrocarbon group such as a norbornane-1,4-diyl group, a norbornane-2,5-diyl group, an adamantane-1,5-diyl group, and an adamantane-2,6-diyl group.

The bonding position of A^{x} to the sultone ring can be any position, but the 1st position is preferable.

Examples of the structural unit (a6-0) include the following structural unit.

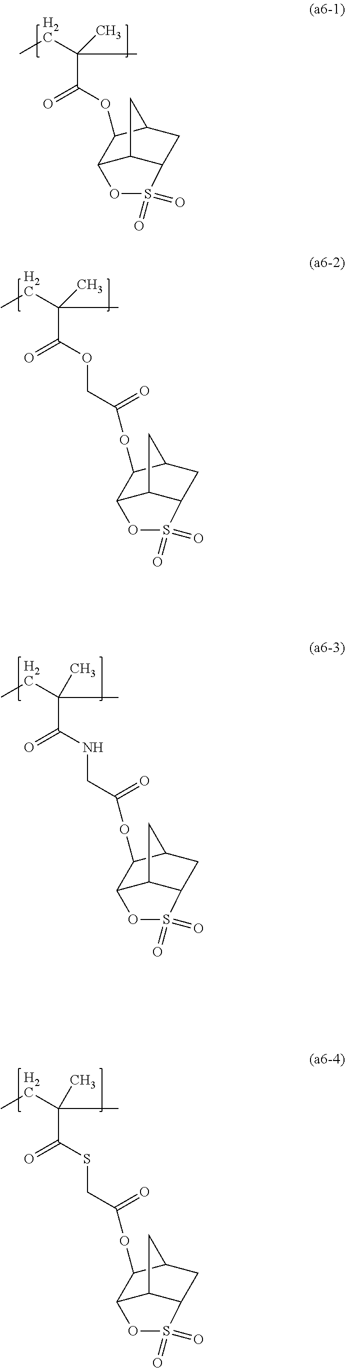

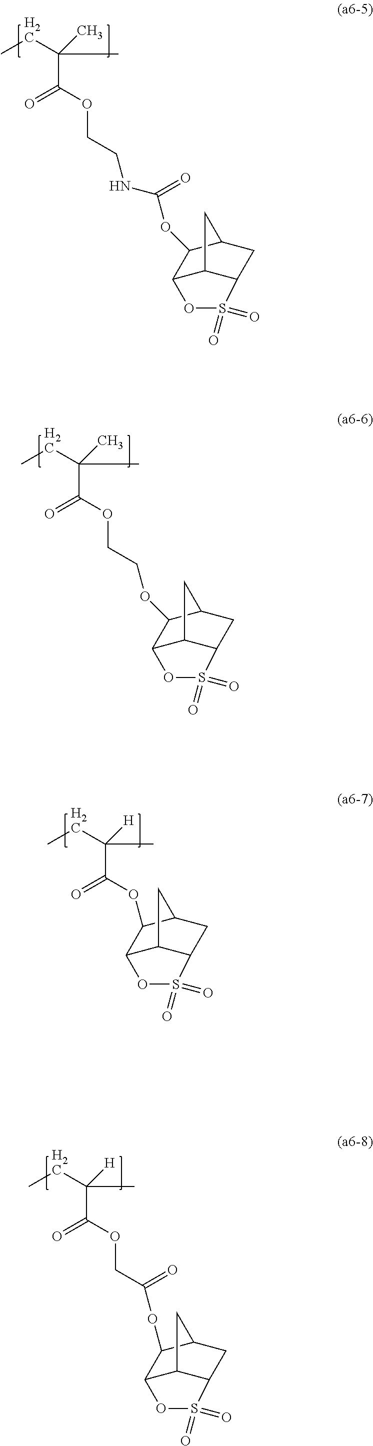

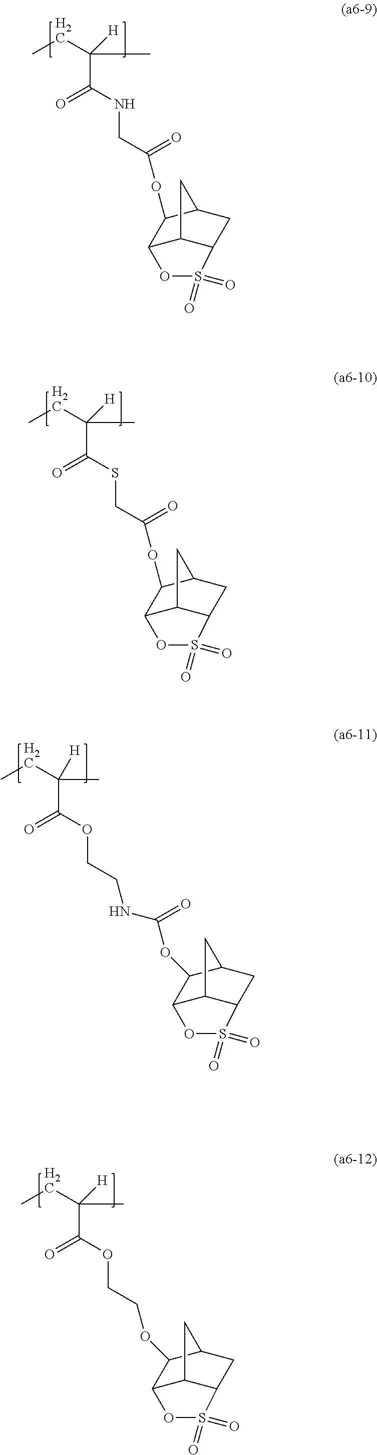

Among these, structural units represented by formulas (a6-1), (a6-2), (a6-6), (a6-7), (a6-8), and (a6-12) are preferable, and structural units represented by formulas (a6-1), (a6-2), (a6-7), and (a6-8) are more preferable.

When the resin (A) etc. includes a structural unit (a6), the content ratio thereof is preferably 1 to 50 mol %, more preferably 2 to 40 mol %, and further preferably 3 to 30 mol %, with respect to the total structural units of the resin (A) etc.

<Structural Unit (a7)>

The resin (A) etc. may further contain a structural unit that decomposes upon exposure to generate acid (hereinafter, may be referred to as “structural unit (a7)”). The structural unit (a7) functions in the resist composition in the same manner as an acid generating agent. Specific examples of the structural unit (a7) include the structural units described in Japanese Patent Laid-Open No. 2016-79235, and it is preferable that the structural unit (a7) is a structural unit having a sulfonate anion or a carboxylate anion and an organic cation in a side chain, or a structural unit having a sulfonium cation and an organic anion in a side chain.

The structural unit having a sulfonate anion or a carboxylate anion and an organic cation in a side chain is preferably a structural unit represented by formula (a7-A).

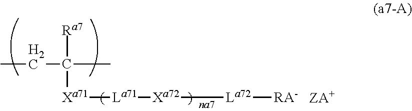

wherein in formula (a7-A),
R^{a7} represents a hydrogen atom, a halogen atom, or an alkyl group having 1 to 6 carbon atoms and optionally having a halogen atom;
X^{a71} represents a single bond, —O—, —CO—, —S—, —SO—, —SO₂—, —NR^{a7}—, a phenylene group optionally having a substituent, or a combined group of these groups;
R^{a71} represents a hydrogen atom or an alkyl group having 1 to 6 carbon atoms;
L^{a71} and L^{a72} each independently represent a hydrocarbon group having 1 to 24 carbon atoms and optionally having a substituent, and —CH₂— contained in the hydrocarbon group is optionally replaced with —O—, —S—, —CO—, —SO—, or —SO₂—;
X^{a72} represents ***—CO—O—, ***—O—CO—, ***—O—CO—O—, or ***—O—, where *** represents the bonding site with L^{a71}
na7 represents an integer of 0 to 2, and when na7 is 2, a plurality of groups in the parentheses are the same or different from each other;
RA⁻ represents a sulfonate anion or a carboxylic acid anion.
ZA⁺ represents an organic cation.

Examples of R^{a7} include the same groups as those exemplified for R^{a01} in formula (a1-0). Among these, R^{a7} is preferably a hydrogen atom or an alkyl group having 1 to 4 carbon atoms, more preferably a hydrogen atom or an alkyl group having 1 to 3 carbon atoms, further preferably a hydrogen atom, a methyl group, or an ethyl group, and still more preferably a hydrogen atom or a methyl group.

Examples of the alkyl group having 1 to 6 carbon atoms for R^{a71} include a methyl group, an ethyl group, an n-propyl group, an isopropyl group, an n-butyl group, a sec-butyl group, a tert-butyl group, a pentyl group, and a hexyl group.

X^{a71} is preferably a single bond, —O—, —CO—, —NR^{a71}—, a phenylene group optionally having a substituent, or a combined group of these groups, and more preferably a single bond, *—O—**, *—CO—**, *—O—CO—O—**, *—CO—NR^{a71}—**, *—NR^{a71}—CO—O—**, *—O—CO—NR^{a71}—**, or *-Ax-Ph-Ay-**.

In the above groups, * and ** represent bonding sites, and * represents the bonding site with the carbon atom to which R^{a7} is bonded. Ax and Ay each independently represent one or more types of bonds selected from the group consisting of a single bond, an ether bond, a thioether bond, an amide bond, an ester bond, and a carbonate ester bond. Ph represents a phenylene group optionally having a substituent.

When X^{a71} is a group represented by *-Ax-Ph-Ay-**, X^{a71} is preferably a linking group represented by the following formula (X10).

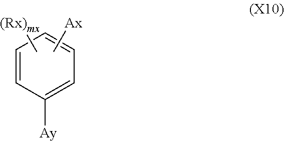

[wherein in formula (X10),
Ax and Ay represent the same meanings as above.
R^{x} represents a halogen atom, a hydroxy group, a fluorinated alkyl group having 1 to 6 carbon atoms, an alkyl group having 1 to 18 carbon atoms, or an alkoxy group having 1 to 6 carbon atoms; and
mx represents an integer of 0 to 4, and when mx is an integer of 2 or more, a plurality of R^{x} are the same or different from each other.

When either A^{x} or A^{y} is a single bond, the other is preferably one or more selected from the group consisting of an ether bond, a thioether bond, an ester bond, a carbonate ester bond, and an amide bond.

When either A^{x} or A^{y} is an amide bond, a bond represented by —CO—NR^{a71}— is preferable.

The bonding position of A in the phenylene group is preferably the m-position or p-position with respect to the bonding position of A^{x}, and more preferably the p-position.

Among these, R^{x} is preferably a fluorine atom, an iodine atom, a trifluoromethyl group, a methyl group, or an ethyl group.

mx is preferably 0, 1, or 2.

Examples of X^{a71} include a single bond and groups represented by the following formulas (X¹⁰-1) to (X¹⁰-10). X²⁰ represents —O— or —NR^{a71}—.

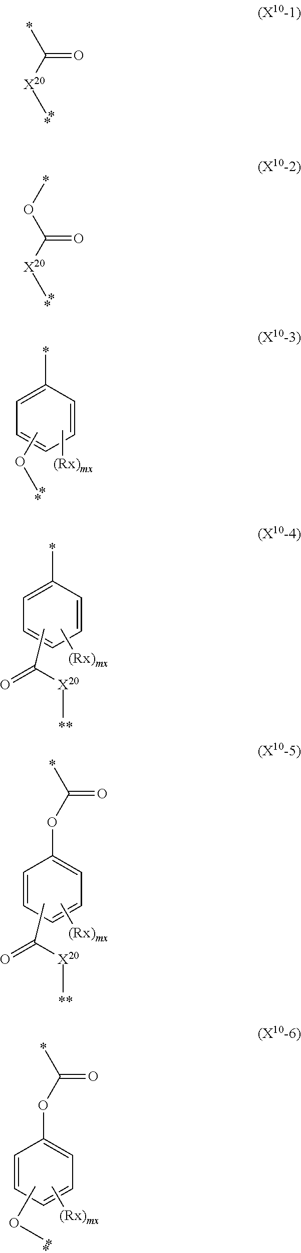

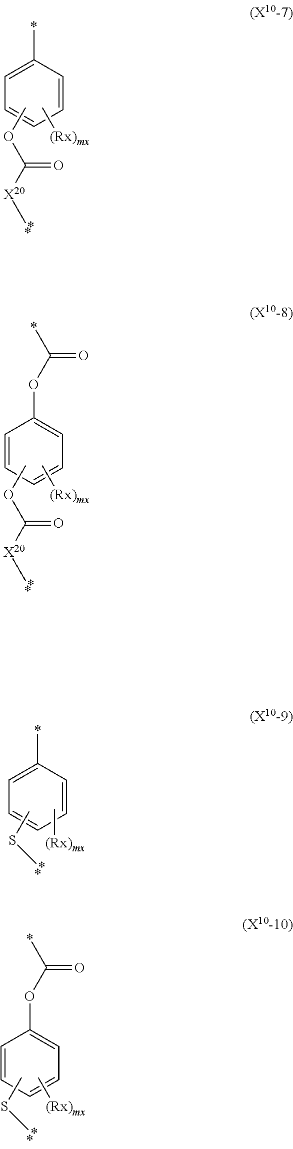

Specific examples of the groups represented by formulas (X¹⁰-1) to (X¹⁰-10) include the following groups.

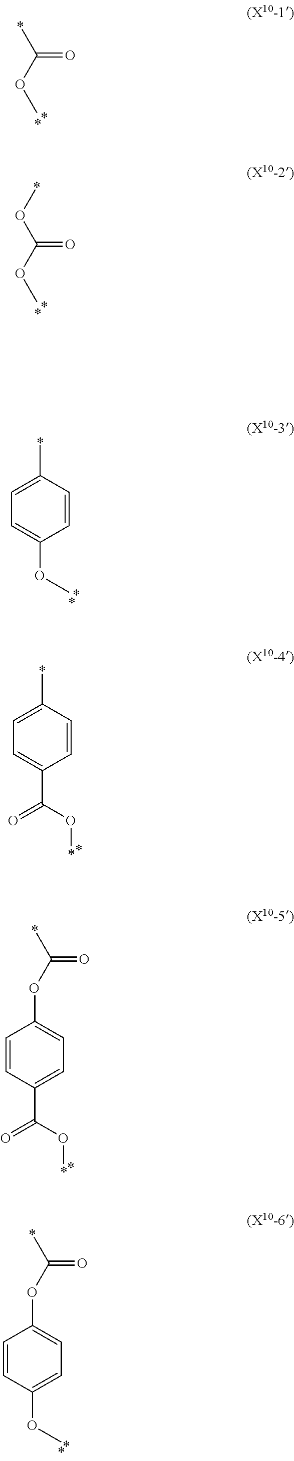

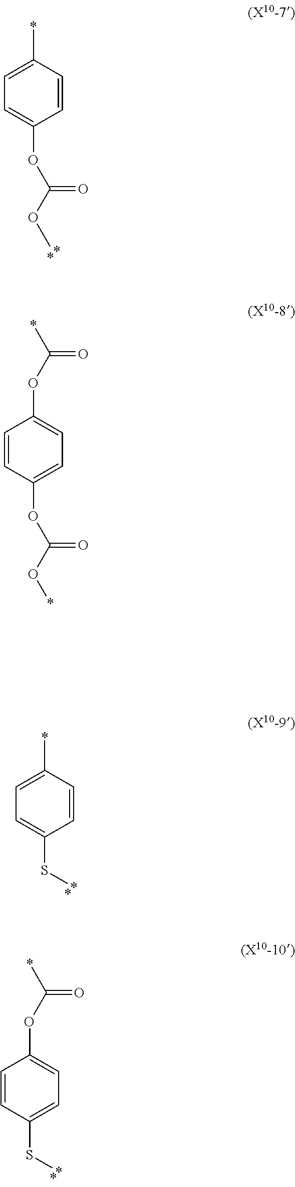 [SMILES: *c1ccc(OC(=O)OC)cc1 ; *OC(=O)Oc1ccc(OC(*)=O)cc1 ; *Sc1ccc(*)cc1 ; *Sc1ccc(OC(*)=O)cc1]

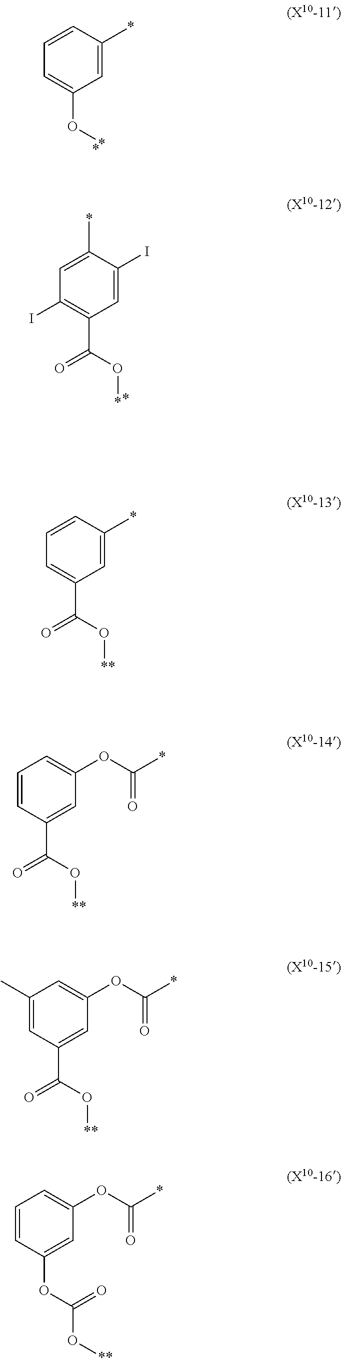

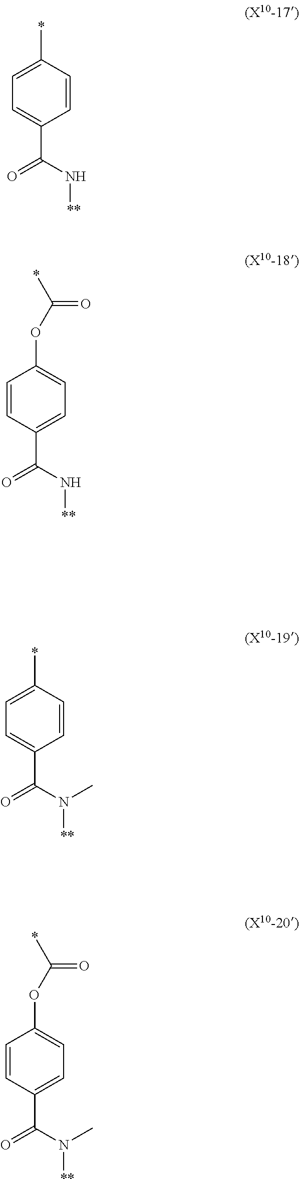

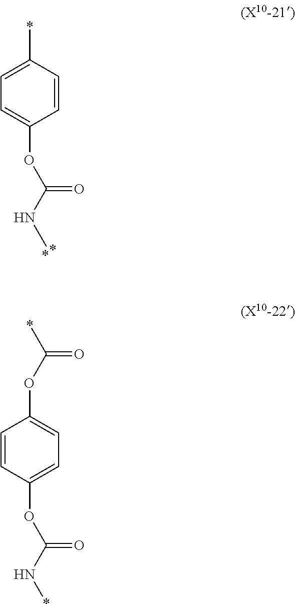 [SMILES: *c1ccc(OC(=O)NC)cc1 ; *NC(=O)Oc1ccc(OC(*)=O)cc1]

Among these, X^{a71} is preferably a single bond or a group represented by any one of formulas (X¹⁰-1′), (X¹⁰-3′) to (X¹⁰-10′), more preferably a single bond or a group represented by any one of formulas (X¹⁰-1′), (X¹⁰-4′), (X¹⁰-5′), (X¹⁰-6′) and (X¹⁰-10′), and further preferably a single bond, a group represented by formula (X¹⁰-1′), a group represented by formula (X¹⁰-5′) or a group represented by formula (X¹⁰-6′).

Examples of the hydrocarbon group for L^{a71} include a group formed by removing one hydrogen atom from a chain hydrocarbon group, an alicyclic hydrocarbon group, an aromatic hydrocarbon group, or a combined group of two or more of these groups, the formed group being bonded to X^{a71} and X^{a72}. Examples of the hydrocarbon group for L^{a72} include a group formed by removing one hydrogen atom from a chain hydrocarbon group, an alicyclic hydrocarbon group, an aromatic hydrocarbon group, or a combined group of two or more of these groups, the formed group being bonded to X^{a72} and RA⁻.

Examples of the chain hydrocarbon group for L^{a71} and L^{a72} include a group formed by removing one hydrogen atom from an alkyl group or an alkenyl group. The alkyl group may be straight-chain or branched, and specific examples thereof include a methyl group, an ethyl group, an n-propyl group, an isopropyl group, an n-butyl group, a sec-butyl group, an isobutyl group, a tert-butyl group, a pentyl group, a hexyl group, a heptyl group, an octyl group, a nonyl group, a decanyl group, an undecanyl group, a dodecanyl group, a tridecanyl group, a tetradecanyl group, a pentane decanyl group, and a heptadecanyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms in the chain hydrocarbon group is preferably 1 to 20, more preferably 1 to 18, and further preferably 1 to 10.

Examples of the alicyclic hydrocarbon group of L^{a71} and L^{a72} include a group in which one hydrogen atom has been removed from a monocyclic or polycyclic cycloalkyl group. Examples of the monocyclic cycloalkyl group include a cyclobutyl group, a cyclopentyl group, a cyclohexyl group, a cycloheptyl group, and a cyclooctyl group.

Examples of the polycyclic cycloalkyl group of L^{a71} and L^{a72} include a cycloalkyl group having a cross-linked structure, a cycloalkyl group in which two or more rings are condensed, or a cycloalkyl group in which two rings are bonded by a spiro bond. Examples of the cycloalkyl group having a cross-linked structure include a norbornyl group and an adamantyl group. Examples of the cycloalkyl group in which two or more types of rings are condensed include a bicyclo[4.4.0]decanyl group and a steroid group (steroid skeleton). Examples of the cycloalkyl group in which two rings are bonded by spiro include a spirocyclic cycloalkyl group in which one type of cycloalkyl group selected from the group consisting of a cyclopentyl group, a cyclohexyl group, a norbornyl group and an adamantyl group, and a cycloalkyl group having 5 to 8 carbon atoms are bonded by spiro. A double bond may be formed between two carbon atoms included in the alicyclic hydrocarbon group. More specifically, examples thereof include alicyclic hydrocarbon groups represented by the following formulae.

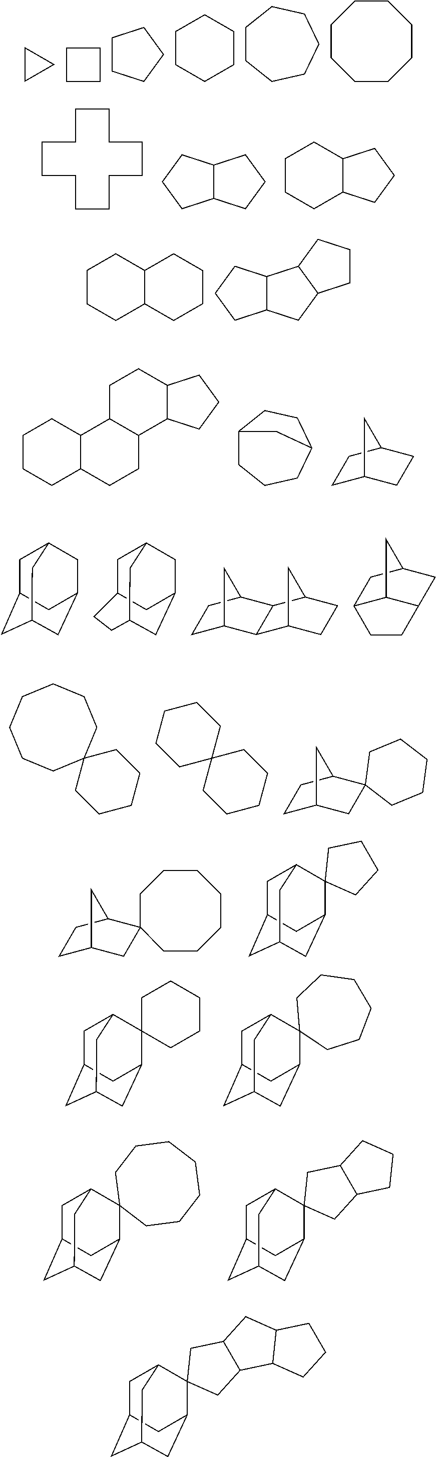

When the alicyclic hydrocarbon group is a monocyclic cycloalkyl group, the number of carbon atoms of the alicyclic hydrocarbon group is preferably 3 to 18, more preferably 3 to 12, and further preferably 3 to 8. When the alicyclic hydrocarbon group is a polycyclic cycloalkyl group, the number of carbon atoms of the alicyclic hydrocarbon group is preferably 6 to 18, and more preferably 7 to 12.

Examples of the aromatic hydrocarbon group of L^{a71} and L^{a72} include an aryl group from which one hydrogen atom has been removed. Examples of the aryl group include a phenyl group, a naphthyl group, an anthryl group, a biphenyl group and a fluorenyl group. The number of carbon atoms in the aromatic hydrocarbon group is preferably 5 to 14, more preferably 6 to 14, and further preferably 6 to 10.

When —CH₂— contained in the hydrocarbon groups for L^{a71} and L^{a72} is replaced with —O—, —CO—, —S—, or —SO₂—, the number of carbon atoms before replacement corresponds to the defined number of carbon atoms in the hydrocarbon group.

Among the hydrocarbon groups for L^{a71} and L^{a72}, examples of the group formed by replacing —CH₂— contained in the chain hydrocarbon group with —O—, —CO—, —S—, or —SO₂— include a hydroxy group, a carboxy group, a carbonyl group, an oxy group, an alkoxy group, an alkoxycarbonyl group, an alkylcarbonyl group, an alkylcarbonyloxy group, an alkanediyloxy group, an alkanediyloxycarbonyl group, an alkanediylcarbonyl group, and an alkanediylcarbonyloxy group. Examples of these replaced groups include the same ones as those exemplified in this specification, within the range permitted by the upper limit of the number of carbon atoms.

Among the hydrocarbon groups of L^{a71} and L^{a72}, examples of the group formed by replacing —CH₂— contained in the alicyclic hydrocarbon group with —O—, —CO—, —S—, or —SO₂— include groups having a structure such as a cyclic ether, a cyclic ketone, a cyclic ester (lactone), a cyclic thioether, a cyclic acetal, and a cyclic sulfonate (sultone). Examples of these replaced groups include the same groups as those exemplified in the present specification, within the acceptable range of the upper limit of the number of carbon atoms.

Among the hydrocarbon groups of L^{a71} and L^{a72}, —CH₂— included in the aromatic hydrocarbon group may be replaced with —O— or —S—, and examples of the group in which —CH₂— is replaced with —O— or —S— include groups derived from a furan ring or a thiophene ring.

Examples of the combined group of two or more of the chain hydrocarbon group, the alicyclic hydrocarbon group, and the aromatic hydrocarbon group for L^{a71} and L^{a72} include a combined group of the above chain hydrocarbon group and the above alicyclic hydrocarbon group, a combined group of the above chain hydrocarbon group and the above aromatic hydrocarbon group, a combined group of the above alicyclic hydrocarbon group and the above aromatic hydrocarbon group, a combined group of the above chain hydrocarbon group, the above alicyclic hydrocarbon group, and the above aromatic hydrocarbon group, and the above combined groups. The combined group of an alicyclic hydrocarbon group and an aromatic hydrocarbon group may be a condensed ring.

Examples of the substituent that the hydrocarbon groups for L^{a71} and L^{a72} optionally have include a halogen atom, a cyano group, and a nitro group. Examples of the halogen atom include a fluorine atom, a chlorine atom, a bromine atom and an iodine atom.

When L^{a71} and L^{a72} are combined groups of an alicyclic hydrocarbon group or an aromatic hydrocarbon group and a chain hydrocarbon group, the chain hydrocarbon group may be substantially regarded as a substituent possessed by the alicyclic hydrocarbon group or the aromatic hydrocarbon group. In addition, —CH₂— in the chain hydrocarbon group included in the hydrocarbon group for L^{a71} and L^{a72} is replaced with —O—, —CO—, —S—, or —SO₂—, whereby the hydrocarbon group for L^{a71} and L^{a72} can substantially have a substituent such as a hydroxy group, a carboxy group, an alkoxy group, an alkoxycarbonyl group, an alkylcarbonyl group, an alkylcarbonyloxy group, a thiol group, an alkylthio group, or an alkylsulfonyl group.

na7 is preferably 0 or 1.

In formula (a7-A), examples of ZA⁺ include the same as the cation in the salt represented by formula (B1).

In formula (a7-A), when the hydrocarbon groups of L^{a71} and L^{a72} are saturated hydrocarbon groups, the same groups as the groups exemplified as the divalent linking group of A¹⁷ in formula (a7-B) described below may be included.

Examples of the structural unit represented by formula (a7-A) include the structural unit and the like represented by formula (a7-A1).

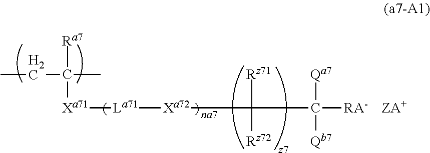

wherein in formula (a7-A1)
R^{a7}, X^{a71}, L^{a71}, X^{a72}, na7, RA⁻, and ZA⁺ have the same meanings as above;
z7 represents an integer of 0 to 6; and
Q^{a7}, Q^{b7}, R^{z71}, and R^{z72} each independently represent a hydrogen atom, a fluorine atom, a perfluoroalkyl group having 1 to 6 carbon atoms, or an alkyl group having 1 to 6 carbon atoms, and when z7 is 2 or more, a plurality of R^{z71} and R^{z72} may be the same or different from each other.

Examples of the perfluoroalkyl group having 1 to 6 carbon atoms or the alkyl group having 1 to 6 carbon atoms in Q^{a7}, Q^{b7}, R^{z71}, and R^{z72} include the same groups as those exemplified for R^{a7}.

Examples of the structural unit represented by formula (a7-A) include the following structural unit, a structural unit in which the group corresponding to the methyl group of R^{a7} is replaced with a hydrogen atom, a halogen atom (e.g., a fluorine atom) or an alkyl group having 1 to 6 carbon atoms and optionally having a halogen atom (e.g., a trifluoromethyl group), and structural units disclosed in WO 2012/050015. ZA⁺ represents an organic cation.

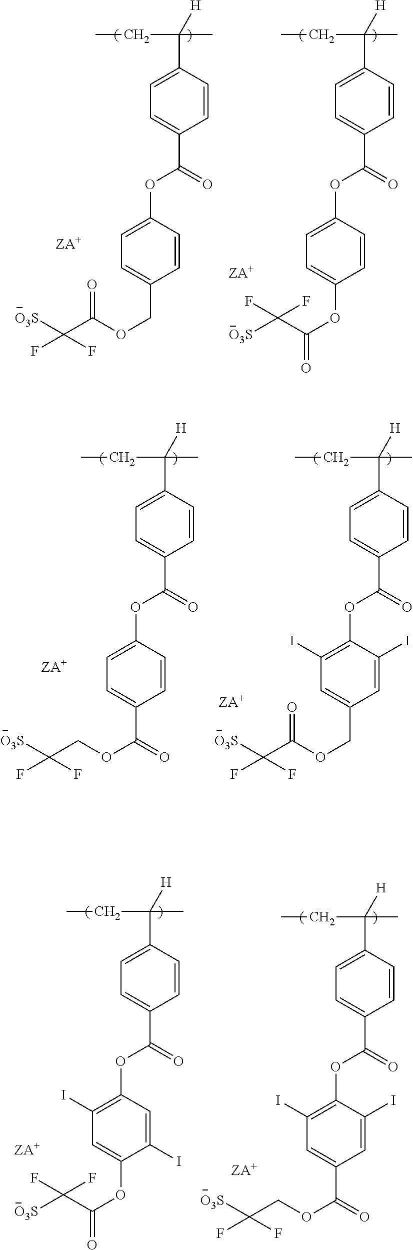

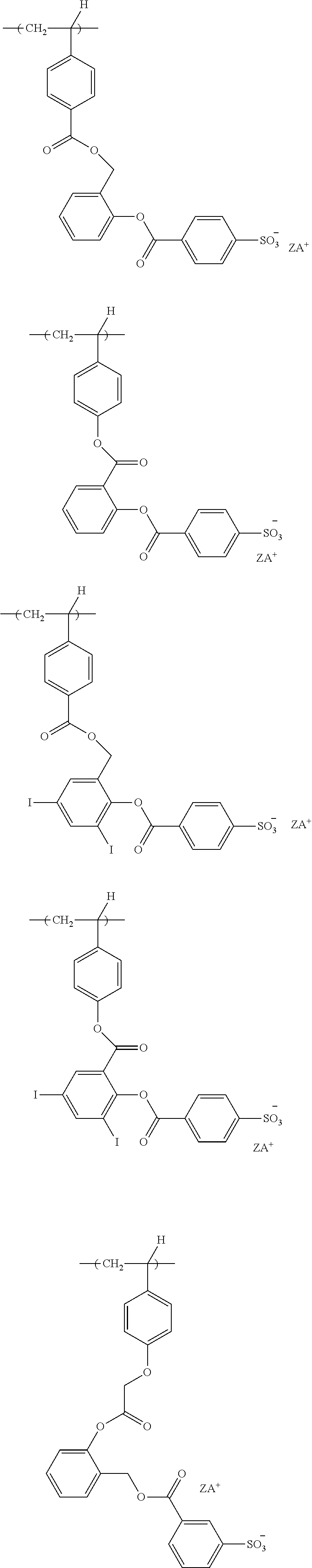

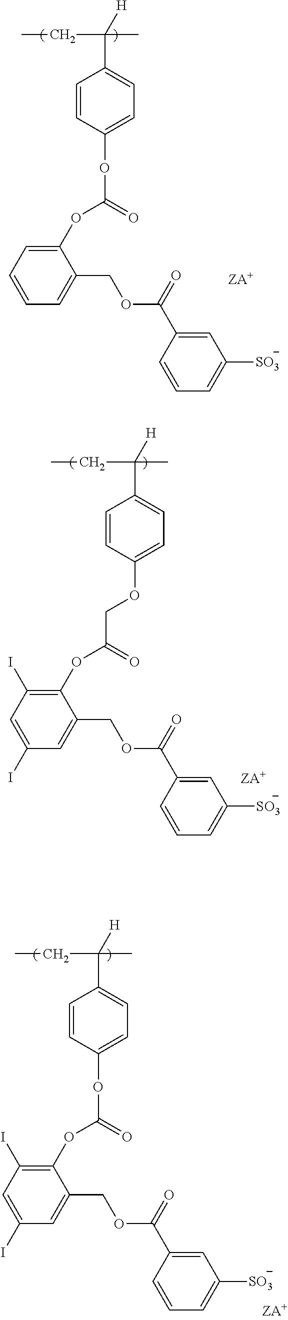

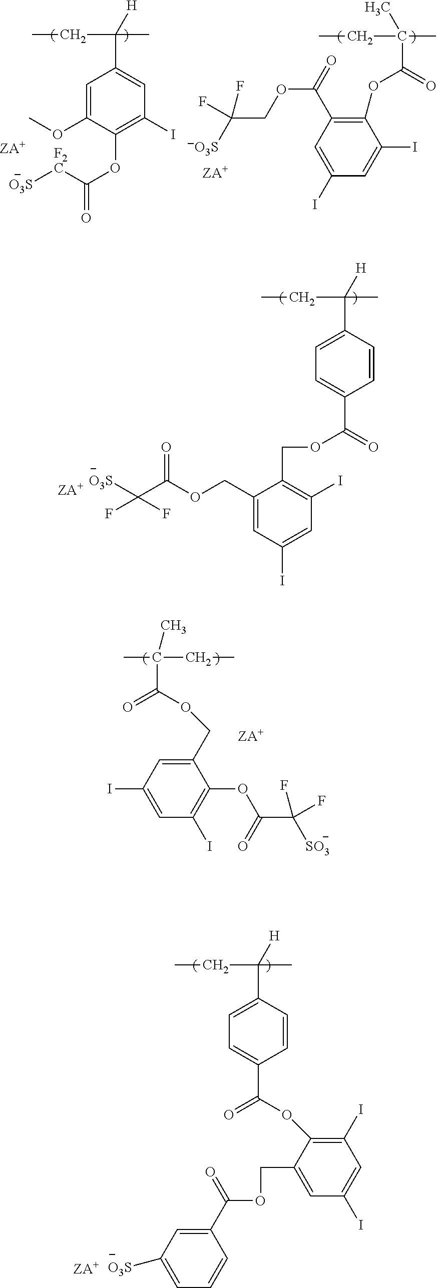

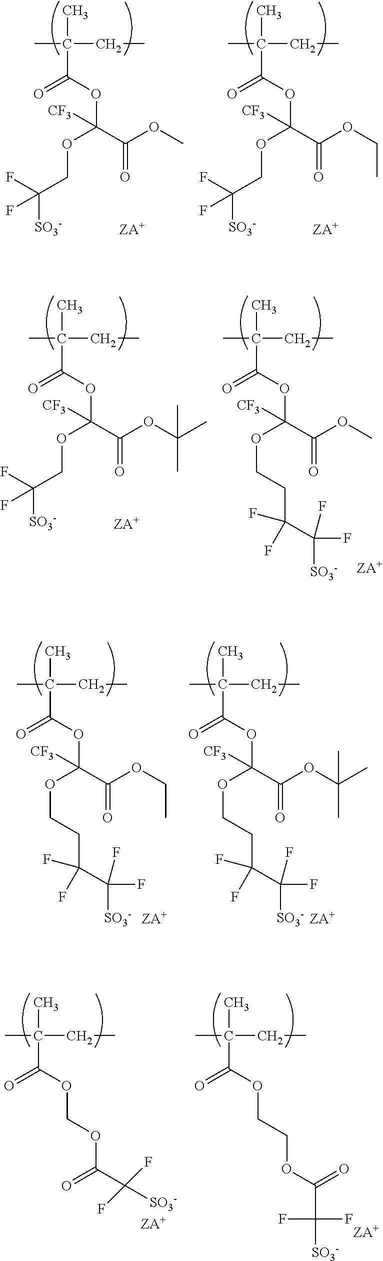

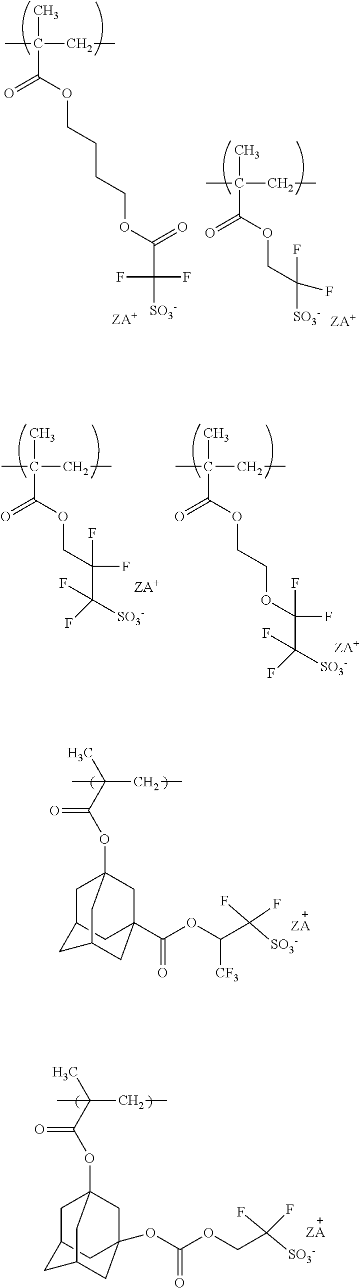

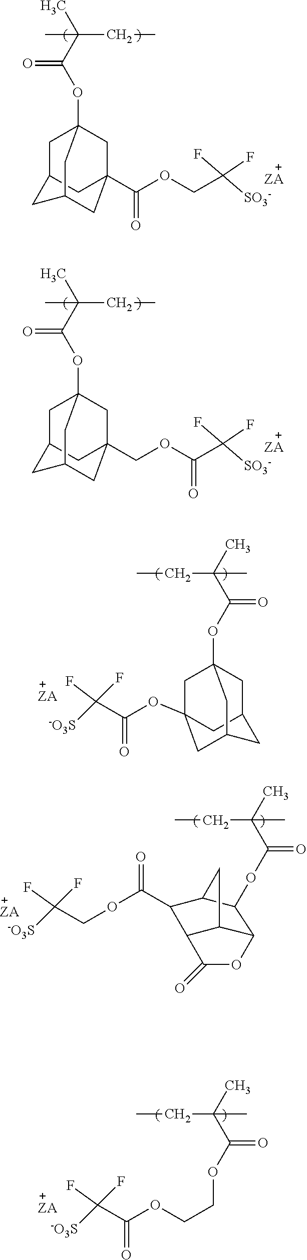

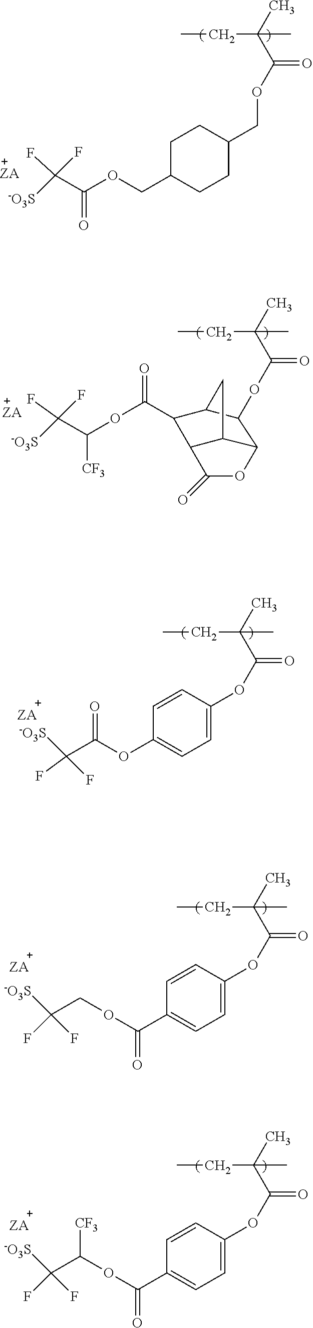

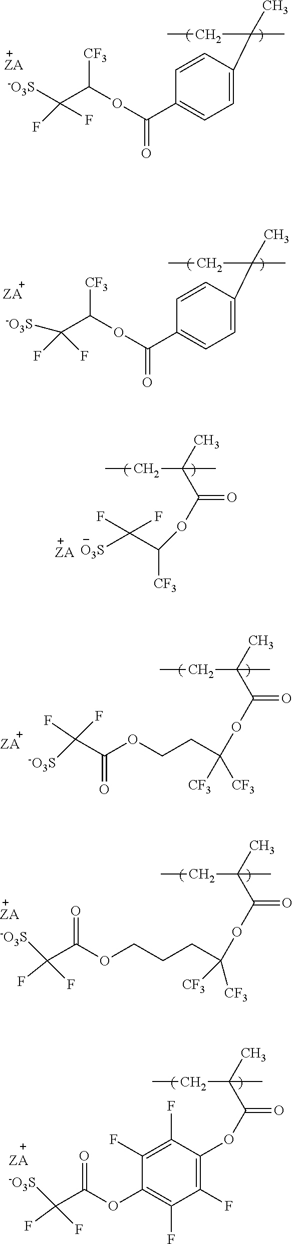

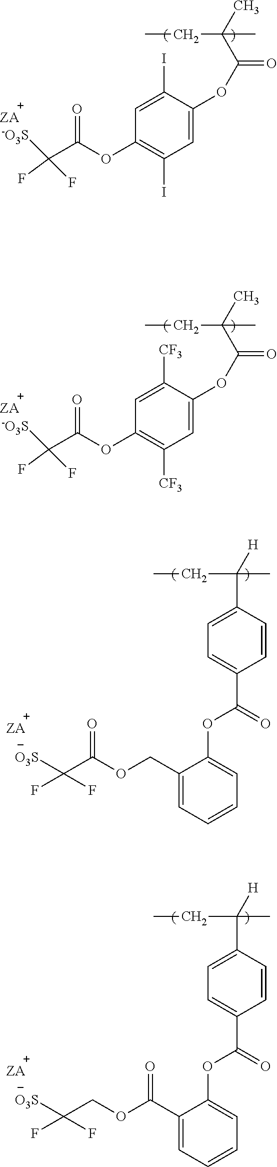

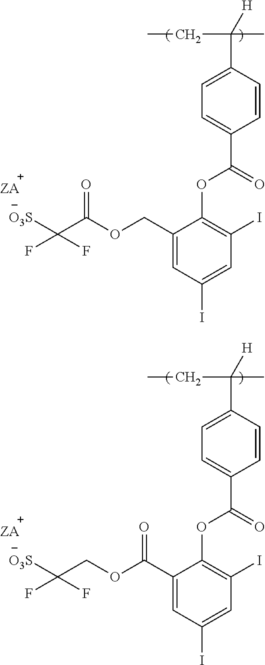

The structural unit having a sulfonio group and an organic anion in the side chain is preferably a structural unit represented by formula (a7-B).

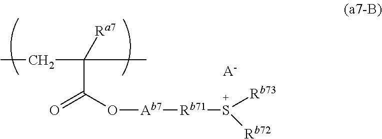

wherein in formula (a7-B),
R^{a7} represents a hydrogen atom, a halogen atom, or an alkyl group having 1 to 6 carbon atoms and optionally having a halogen atom;
A^{b7} represents a single bond or a divalent linking group;
R^{b71} represents a divalent aromatic hydrocarbon group having 6 to 18 carbon atoms and optionally having a substituent;
R^{b72} and R^{b73} each independently represent a hydrocarbon group having 1 to 18 carbon atoms and optionally having a substituent, and R^{b72} and R^{b73} may be bonded to each other to form a ring together with the sulfur atom to which they are bonded;
A⁻ represents an organic anion.

Examples of the halogen atom and alkyl group optionally having a halogen atom of R^{a7} include the same halogen atom and alkyl group optionally having a halogen atom as those of formula (a7-A).

Examples of the divalent aromatic hydrocarbon group having 6 to 18 carbon atoms represented by R^{b71} include a phenylene group and a naphthylene group.

Examples of the hydrocarbon group represented by R^{b72} and R^{b73} include an alkyl group, an alicyclic hydrocarbon group, an aromatic hydrocarbon group, and a group formed by combining these groups.

Examples of the alkyl group, the alicyclic hydrocarbon group, the aromatic hydrocarbon group, and the group formed by combining these groups include the same as those described above.

Examples of the divalent linking group represented by A^{b7} include a divalent saturated hydrocarbon group having 1 to 18 carbon atoms, and —CH₂— contained in the divalent saturated hydrocarbon group is optionally replaced with —O—, —S—, —SO—, —SO₂—, or —CO—.

Examples of the divalent saturated hydrocarbon group include divalent chain saturated hydrocarbon groups such as a straight-chain or branched alkanediyl group, a monocyclic or polycyclic divalent alicyclic saturated hydrocarbon group, and a combination thereof.

Specific examples thereof include: a straight-chain alkanediyl group such as a methylene group, an ethylene group, a propane-1,3-diyl group, a propane-1,2-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and a dodecane-1,12-diyl group; a branched alkanediyl group such as a butane-1,3-diyl group, a 2-methylpropane-1,3-diyl group, a 2-methylpropane-1,2-diyl group, a pentane-1,4-diyl group, and a 2-methylbutane-1,4-diyl group; a divalent monocyclic alicyclic saturated hydrocarbon group that is a monocyclic cycloalkanediyl group, such as a cyclobutane-1,3-diyl group, a cyclopentane-1,3-diyl group, a cyclohexane-1,4-diyl group, and a cyclooctane-1,5-diyl group; and a divalent polycyclic alicyclic saturated hydrocarbon group that is a polycyclic cycloalkanediyl group, such as a norbornane-1,4-diyl group, a norbornane-2,5-diyl group, an adamantane-1,5-diyl group, and an adamantane-2,6-diyl group.

Examples of the saturated hydrocarbon group in which —CH₂— has been replaced with —O—, —S—, —SO—, —SO₂—, or —CO— include the divalent groups shown below. Provided that the number of carbon atoms before —CH₂— in the saturated hydrocarbon group is replaced with —O—, —S—, —SO—, —SO₂—, or —CO— is 17 or less. In the formula below, * and ** represent bonding sites, and * represents the bonding site with R^{b71}.

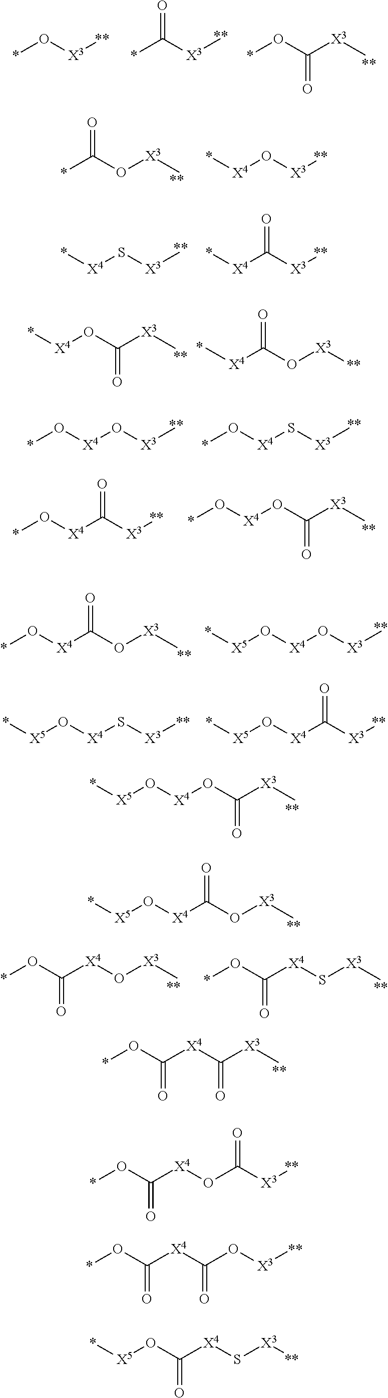

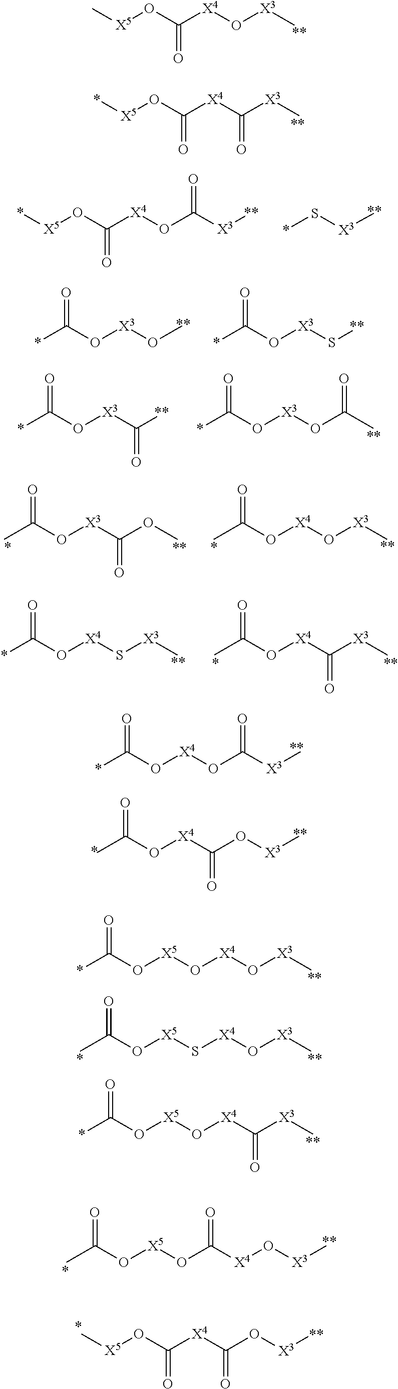

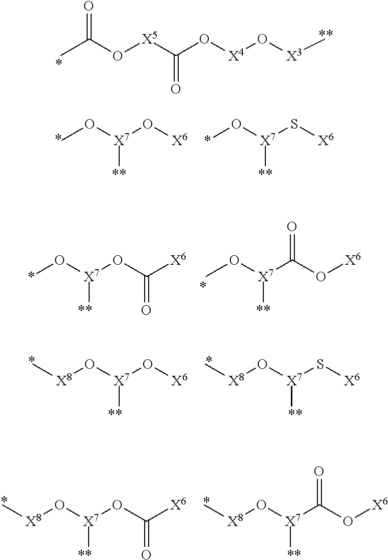

wherein X³ represents a divalent saturated hydrocarbon group having 1 to 16 carbon atoms;
X⁴ represents a divalent saturated hydrocarbon group having 1 to 15 carbon atoms;
X⁵ represents a divalent saturated hydrocarbon group having 1 to 13 carbon atoms;
X⁶ represents a divalent saturated hydrocarbon group having 1 to 14 carbon atoms;
X⁷ represents a trivalent saturated hydrocarbon group having 1 to 14 carbon atoms; and
X⁸ represents a divalent saturated hydrocarbon group having 1 to 13 carbon atoms.

Examples of the structural unit including a cation in formula (a7-B) include structural units represented by the following formulas and structural units in which the group corresponding to the methyl group in R^{a7} is replaced with a hydrogen atom, a halogen atom (e.g., a fluorine atom), or an alkyl group having 1 to 6 carbon atoms and optionally having a halogen atom (e.g., a trifluoromethyl group).

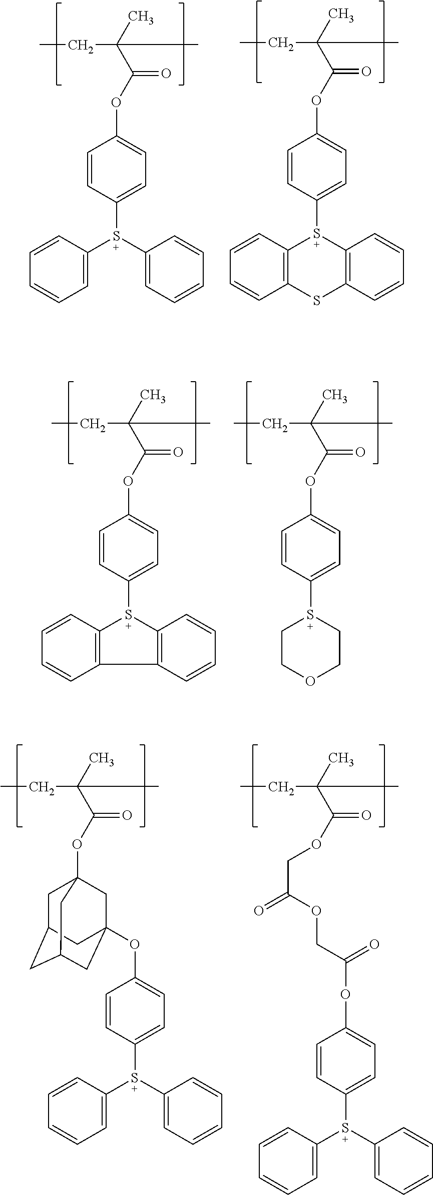

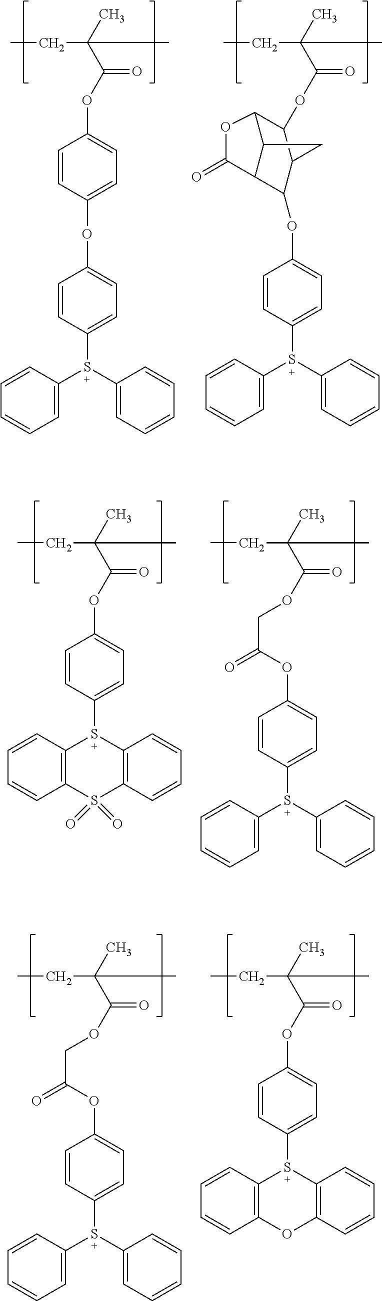

Examples of the organic anion represented by A⁻ include a sulfonate anion, a sulfonylimide anion, a sulfonylmethide anion, and a carboxylate anion. The organic anion represented by A⁻ is preferably a sulfonate anion, and the sulfonate anion is more preferably the same anions as those exemplified in the acid generating agent (B). Examples of the sulfonylimide anion, sulfonylmethide anion, and carboxylate anion include the same anions as those exemplified in the acid generating agent (B).

Examples of the structural unit represented by formula (a7-B) include the structural units represented below.

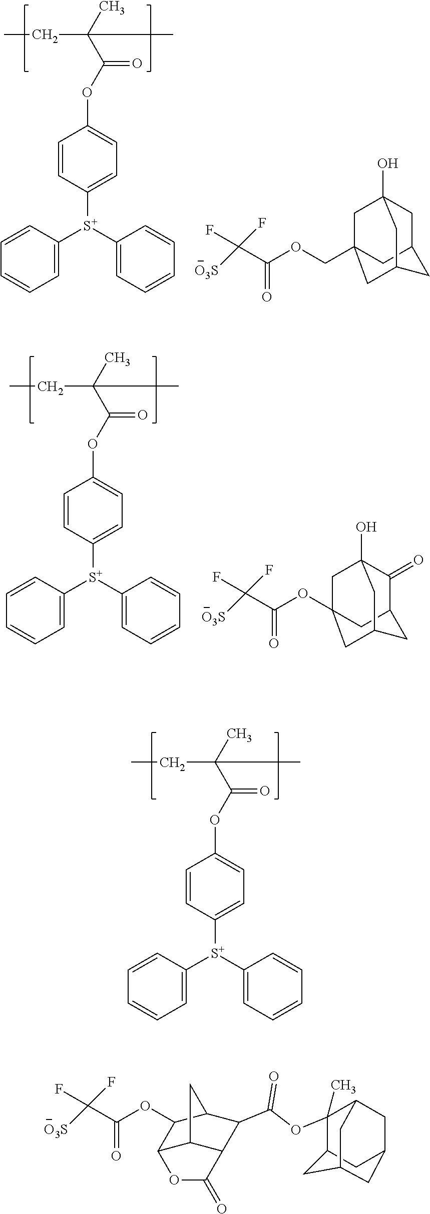

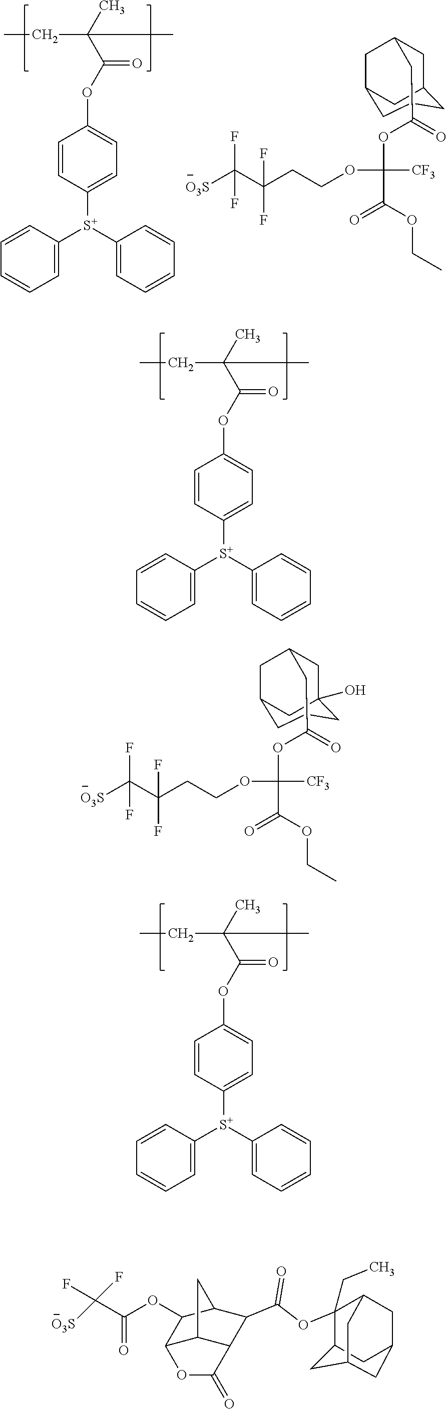

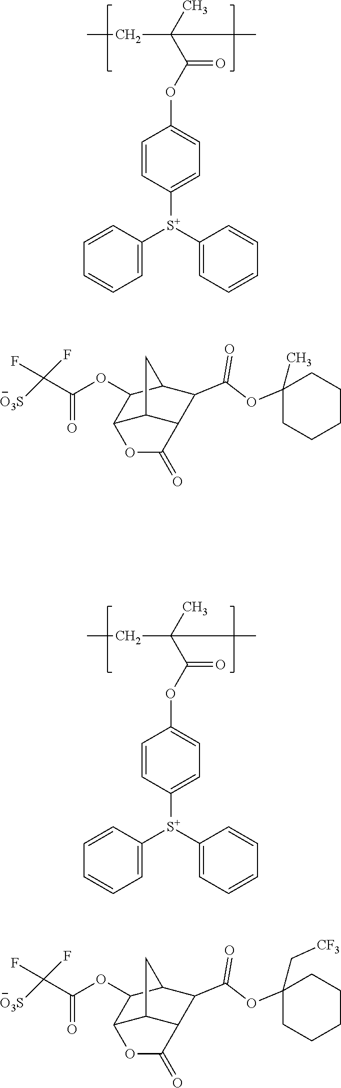

When the resin (A) etc. includes the structural unit (a7), the content ratio of the structural unit (a7) is preferably 1 to 30 mol %, more preferably 2 to 25 mol %, and further preferably 3 to 20 mol %, with respect to the total structural units of the resin (A) etc.

The resin (A) etc. is preferably a resin that contains the structural unit (a1). In particular, the resin (Ap) is more preferably a resin consisting of the structural unit (IP), the structural unit (a1), and the structural unit (s), that is, a copolymer of the salt (I), the monomer (a1), and the monomer (s). The resin (A) that does not contain the structural unit (IP) is preferably a resin consisting of the structural unit (a1) and the structural unit (s), that is, a copolymer of the monomer (a1) and the monomer (s).

The structural unit (a1) is preferably at least one selected from the group consisting of the structural unit (a1-0), the structural unit (a1-1), the structural unit (a1-2) (preferably the structural unit having a cyclohexyl group or a cyclopentyl group), the structural unit represented by formula (a1-4), the structural unit represented by formula (a1-5), and the structural unit represented by formula (a1-6), and more preferably at least one selected from the group consisting of the structural unit (a1-1) and the structural unit (a1-2).

The structural unit (s) is preferably at least one selected from the group consisting of the structural unit (a2) and the structural unit (a3). The structural unit (a2) is preferably the structural unit (a2-C) or the structural unit (a2-A). The structural unit (a3) is preferably at least one selected from the group consisting of a structural unit represented by formula (a3-1), a structural unit represented by formula (a3-2), and a structural unit represented by formula (a3-4).

Each structural unit constituting the resin (A) etc. may be used singly or in combination of two or more, and can be produced by known polymerization methods (e.g., radical polymerization methods) using monomers that lead to these structural units. The content ratio of each structural unit in the resin (A) etc. can be adjusted by the amount of monomer used in polymerization.

The resin (A) and the resin (Ap) are, for example, polymers having a weight average molecular weight of preferably 2,000 or more (more preferably 2,500 or more, and further preferably 3,000 or more) and 50,000 or less (more preferably 30,000 or less, and further preferably 15,000 or less), but may contain an oligomer having a weight average molecular weight of less than 2,000. In this specification, the weight average molecular weight is a value determined by gel permeation chromatography under the conditions described in the Examples. The structural unit (IP) may constitute a dimer, a trimer, a compound having a weight average molecular weight of less than 2,000, or an oligomer.

### <Acid Generating Agent>

The acid generating agent of the present invention is an acid generating agent containing the salt (I) or the structural unit (IP) of the present invention. The structural unit (IP) can be contained as a compound or resin (Ap) in which a plurality of units are polymerized. In the acid generating agent, it may contain one type of salt (I) or two or more types of salts (I). One type of compound or resin containing the structural unit (IP) may be used alone, or two or more types thereof may be used in combination. Moreover, the acid generating agent of the present invention may contain both the salt (I) and the structural unit (IP).

The acid generating agent of the present invention may contain, in addition to the salt (I) and/or the structural unit (IP), a known compound (hereinafter referred to as “compound (B)” or “acid generating agent (B)” in some cases) which acts as an acid generating agent, in the resist field. The compound (B) may be used alone or in combination of two or more types. The compound (B) will be described later.

When the acid generating agent contains the salt (I) and the compound (B), a ratio between the content of the salt (I) and the content of the compound (B) (mass ratio; salt (I): compound (B)) is usually 1:99 to 99:1, is preferably 2:98 to 98:2, is more preferably 5:95 to 95:5, is further preferably 10:90 to 90:10, is even still more preferably 15:85 to 85:15, and is particularly preferably 40:60 to 60:40.

When the acid generating agent contains the structural unit (IP) and the compound (B), a ratio between the content of the structural unit (IP) and the content of the compound (B) (mass ratio; structural unit (IP): compound (B)) is usually 1:99 to 99:1, is preferably 2:98 to 98:2, is more preferably 5:95 to 95:5, is further preferably 10:90 to 90:10, is even still more preferably 15:85 to 85:15, and is particularly preferably 40:60 to 60:40.

### [Resist Composition]

The resist composition of the present invention contains the acid generating agent of the present invention. The acid generating agent herein may be the salt (I), or may be the resin (Ap) containing the structural unit (IP). The resist composition of the present invention may contain a resin in addition to the acid generating agent of the present invention. The resin may be a resin that contains the structural unit (a1) having an acid labile group, or may be a resin that does not contain the structural unit (a1). However, the resist composition of the present invention contains at least one of the salt (I) and the structural unit (IP), and may contain both. That is, the resist composition of the present invention may contain an acid generating agent that contains the structural unit (IP) of the present invention or the salt (I) of the present invention. The structural unit (IP) may be in any form, such as a compound or a resin. In other words, the resist composition of the present invention may contain, as an acid generating agent, the resin (Ap) and/or the resin (A), and the salt (I). It is preferable that the resist composition of the present invention contains a resin that includes the structural unit (a1) having an acid labile group. That is, it preferably contains at least
(a) the salt (I), and the resin (A) containing the structural unit (a1) having an acid labile group, or
(b) the resin (Ap) that contains the structural unit (IP) and the structural unit (a1) having an acid labile group, or
(c) the resin (Ap) that contains the structural unit (IP), and the resin (A) containing the structural unit (a1) having an acid labile group.

Among them, it is preferably the resist composition of the above (b). It may contain two or more resins (A) and/or resins (Ap).

The resist composition of the present invention preferably further contains a quencher (hereinafter may be referred to as “quencher (C)” in some cases) and/or a solvent (hereinafter may be referred to as “solvent (E)” in some cases). In addition, the resist composition of the present invention may further contain a resin other than the aforementioned resin (A) etc.

<Resin Other than Resin (A) Etc.>

The resist composition of the present invention may contain a resin other than the resin (Ap) and the resin (A). Examples of the resin other than the resin (Ap) and the resin (A) include a resin that contains the same structural unit as that of the resin (A) except that the above described resin (A) does not contain the structural unit (a1) (hereinafter may be referred to as “resin (AX)” in some cases), and a resin containing the structural unit (a4) and/or the structural unit (a5) (provided that it does not contain the structural unit (IP) and the structural unit (a1). Hereinafter may be referred to as “resin (X)” in some cases).

Examples of the resin (AX) include resins including the structural unit (a2), and resins including the structural unit (a2-A) are preferable. In the resin (AX), the content ratio of the structural unit (a2-A) is preferably 5 mol % or more, more preferably 10 mol % or more, and further preferably 15 mol % or more, with respect to the total of all structural units in the resin (AX). In addition, the content ratio is preferably 80 mol % or less, and more preferably 70 mol % or less.

Examples of structural units that may be further contained in the resin (X) include the structural unit (a2), the structural unit (a3), and structural units derived from other known monomers. Among these, the resin (X) is preferably a resin including only the structural unit (a4) and/or the structural unit (a5), and more preferably a resin including only the structural unit (a4).

When the resin (X) includes the structural unit (a4), the content ratio of the structural unit (a4) in the resin (X) is, with respect to the total of all structural units in the resin (X), 20 mol % or more, preferably 30 mol % or more, more preferably 40 mol % or more, and further preferably 45 mol % or more. In addition, examples of thereof include 100 mol % or less, 80 mol % or less is preferable, 70 mol % or less is more preferable, 60 mol % or less is further preferable, and 55 mol % or less is still more preferable. Specific examples thereof include 20 to 100 mol %, 20 to 80 mol % is preferable, 30 to 70 mol % is more preferable, 40 to 60 mol % is further preferable, and 45 to 55 mol % is still more preferable. When the resin (X) includes a structural unit (a5), examples of the content ratio of the structural unit (a5) include 20 mol % or more with respect to the total of all structural units in the resin (X), 30 mol % or more is preferable, 40 mol % or more is more preferable, and 45 mol % or more is still more preferable. In addition, examples thereof include 100 mol % or less, 80 mol % or less is preferable, 70 mol % or less is more preferable, 60 mol % or less is further preferable, and 55 mol % or less is still more preferable. Specific examples thereof include 20 to 100 mol %, 20 to 80 mol % is preferable, 30 to 70 mol % is more preferable, 40 to 60 mol % is further preferable, and 45 to 55 mol % is still more preferable. In addition, when the resin (X) includes the structural unit (a4) and the structural unit (a5), examples of the total content ratio of the structural unit (a4) and the structural unit (a5) with respect to the total of all structural units in the resin (X) include 40 mol % or more, 60 mol % or more is preferable, 70 mol % or more is more preferable, and 80 mol % or more is further preferable. Furthermore, examples thereof include 100 mol % or less. Specific examples thereof include 40 to 100 mol %, 60 to 100 mol % is preferable, 70 to 100 mol % is more preferable, and 80 to 100 mol % is further preferable.

Each structural unit constituting the resin (AX) and the resin (X) may be used singly or in combination of two or more, and can be produced by a known polymerization method (e.g., radical polymerization method) using monomers that derive these structural units. The content ratio of each structural unit included in the resin (AX) and the resin (X) can be adjusted by the amount of monomer used in polymerization.

The resin (AX) and the resin (X) are polymers having a weight average molecular weight of preferably 6000 or more (more preferably 7000 or more) and 80000 or less (more preferably 60000 or less), but may include an oligomer having a weight average molecular weight of less than 6000. The method for measuring the weight average molecular weight of the resin (AX) and the resin (X) is the same as that for the resin (A) etc.

When the resist composition of the present invention includes the resin (X), the content thereof is preferably 1 to 60 parts by mass, more preferably 1 to 50 parts by mass, further preferably 1 to 40 parts by mass, still more preferably 1 to 30 parts by mass, and even still more preferably 1 to 8 parts by mass, with respect to a total of 100 parts by mass of the resin (A) etc.

The content ratio of the resin (A) etc. in the resist composition is preferably 60% by mass or more and 99% by mass or less, more preferably 70% by mass or more and 99% by mass or less, further preferably 80% by mass or more and 99% by mass or less, and still more preferably 90% by mass or more and 99% by mass or less, with respect to the solid content of the resist composition. The content ratio of the resin (Ap) is preferably 60% by mass or more and 99% by mass or less, more preferably 70% by mass or more and 99% by mass or less, further preferably 80% by mass or more and 99% by mass or less, and still more preferably 90% by mass or more and 99% by mass or less, with respect to the solid content of the resist composition. In addition, when a resin other than the resin (A) etc. is included, the total content ratio of the resin (A) etc. and the resin other than the resin (A) etc. is preferably 60% by mass or more and 99% by mass or less, more preferably 70% by mass or more and 99% by mass or less, further preferably 80% by mass or more and 99% by mass or less, and still more preferably 90% by mass or more and 99% by mass or less, with respect to the solid content of the resist composition. The solid content of the resist composition and the resin content ratio therein can be measured by known analytical means such as liquid chromatography or gas chromatography.

### <Acid Generating Agent>

The acid generating agent of the present invention may be any acid generating agent that contains only a salt (I) and/or a structural unit (IP), or an acid generating agent that contains a salt (I) and/or a structural unit (IP) and a known compound (B) that acts as an acid generating agent in the resist field.

The acid generating agent used in the resist composition of the present invention may be used alone as one kind or may be used in combination of two or more kinds.

### <Compound (B)>

As the compound (B), any one of a nonionic compound or an ionic compound may be used. Examples of the nonionic compound include sulfonate esters (for example, 2-nitrobenzyl ester, aromatic sulfonate, oxime sulfonate, N-sulfonyloxyimide, sulfonyloxyketone and diazonaphthoquinone 4-sulfonate), and sulfones (for example, disulfone, ketosulfone and sulfonyldiazomethane). Typical examples of the ionic compound include an onium salt containing an onium cation (for example, diazonium salt, phosphonium salt, sulfonium salt and iodonium salt). Examples of the anion of the onium salt include a sulfonate anion, a sulfonylimide anion, a sulfonylmethide anion, and a carboxylate anion.

The compound (B) may be a part of the structural units constituting the resins which are contained in the resist composition of the present invention, such as the structural unit (a7) described above.

As the compound (B), there can be used the compounds that generate an acid when exposed to radiation, such as those described in Japanese Patent Laid-Open No. 63-26653, Japanese Patent Laid-Open No. 55-164824, Japanese Patent Laid-Open No. 62-69263, Japanese Patent Laid-Open No. 63-146038, Japanese Patent Laid-Open No. 63-163452, Japanese Patent Laid-Open No. 62-153853, Japanese Patent Laid-Open No. 63-146029, U.S. Pat. Nos. 3,779,778, 3,849,137, German Patent No. 3914407, and European Patent No. 126,712. In addition, the compound produced by a known method may be used.

The compound (B) may be an acid generating agent having an acid labile group. For example, the compound or resin serving as an acid generating agent may contain a partial structure serving as an acid labile group, and the acid labile group preferably contains a group (1) or a group (2) exemplified in the present specification. When the resist composition of the present invention contains a compound having an acid labile group, a resin containing the structural unit having an acid labile group may be omitted.

Preferred embodiments of the compound (B) include a salt represented by formula (B1) (hereinafter may be referred to as “salt (B1)”, “acid generating agent (B1)” or “compound (B1)” in some cases), a salt represented by formula (B2) (hereinafter may be referred to as “salt (B2)”, “acid generating agent (B2)” or “compound (B2)” in some cases), and a salt represented by formula (B4) (hereinafter may be referred to as “salt (B4)”, “acid generating agent (B4)” or “compound (B4)” in some cases).

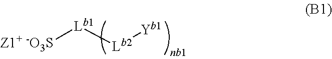

wherein in formula (B1),
L^{b1} represents a single bond or a hydrocarbon group having a valence of (nb1+1) and optionally having a substituent, and —CH₂— contained in the hydrocarbon group is optionally replaced with —O—, —S—, —CO—, —SO— or —SO₂—;
L^{b2} represents a single bond or a divalent hydrocarbon group having 1 to 24 carbon atoms and optionally having a substituent, and —CH₂— contained in the hydrocarbon group is optionally replaced with —O—, —S—, —CO—, —SO— or —SO₂—;
Y^{b1} represents a methyl group optionally having a substituent, or a cyclic hydrocarbon group having 3 to 24 carbon atoms and optionally having a substituent, and —CH₂— contained in the cyclic hydrocarbon group is optionally replaced with —O—, —S—, —SO₂—, —SO— or —CO—;
nb1 represents an integer of 1 to 6; when nb1 is 2 or larger, a plurality of groups in the parentheses may be the same or different from each other;
Z1⁺ represents an organic cation.

Examples of the hydrocarbon group having a valence of (nb1+1) for L^{b1} include a group formed by removing nb1 number of hydrogen atoms from a monovalent chain hydrocarbon group, a monovalent alicyclic hydrocarbon group, a monovalent aromatic hydrocarbon group, or a monovalent combined group of two or more of these groups, the formed group being bonded to one or more L^{b2}. The number of carbon atoms of the hydrocarbon group is preferably 1 to 48, more preferably 1 to 42, further preferably 1 to 36, far more preferably 1 to 30, still further preferably 1 to 24, and even more preferably 1 to 18.

Examples of the chain hydrocarbon group include a group formed by removing nb1 hydrogen atoms from an alkyl group or an alkenyl group. The alkyl group may be a straight-chain or branched group, and specific examples thereof include a methyl group, an ethyl group, an n-propyl group, an iso-propyl group, an n-butyl group, a sec-butyl group, an iso-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group and a heptadecyl group. Examples of the alkenyl group include an ethenyl group, a propenyl group, an isopropenyl group, a butenyl group, an isobutenyl group, a tert-butenyl group, a pentenyl group, a hexenyl group, a heptenyl group, an octenyl group, an isooctenyl group and a nonenyl group.

The number of carbon atoms of the chain hydrocarbon group is preferably 1 to 36, is more preferably 1 to 24, is further preferably 1 to 20, is far more preferably 1 to 18, is still further preferably 1 to 12, and even more preferably 1 to 10.

Examples of the alicyclic hydrocarbon group include such a group that nb1 hydrogen atoms have been removed from a monocyclic type or polycyclic type of cycloalkyl group. Examples of the monocyclic cycloalkyl group include a cyclobutyl group, a cyclopentyl group, a cyclohexyl group, a cycloheptyl group, and a cyclooctyl group.

Examples of the polycyclic type of cycloalkyl group include a cycloalkyl group having a cross-linked structure, a cycloalkyl group in which two or more rings are condensed, and a cycloalkyl group in which two rings are bonded by spiro. Examples of the cycloalkyl group having a cross-linked structure include a norbornyl group and an adamantyl group. Examples of the cycloalkyl group in which two or more types of rings are condensed include a bicyclo[4.4.0]decanyl group and a steroid group (steroid skeleton). Examples of the cycloalkyl group in which two rings are bonded by spiro include a spirocyclic cycloalkyl group in which one or more types of cycloalkyl group selected from the group consisting of a cyclopentyl group, a cyclohexyl group, a norbornyl group and an adamantyl group, and a cycloalkyl group having 5 to 8 carbon atoms are bonded by spiro. A double bond may be formed between two carbon atoms included in the alicyclic hydrocarbon group.

More specifically, examples thereof include alicyclic hydrocarbon groups represented by the following formulae.

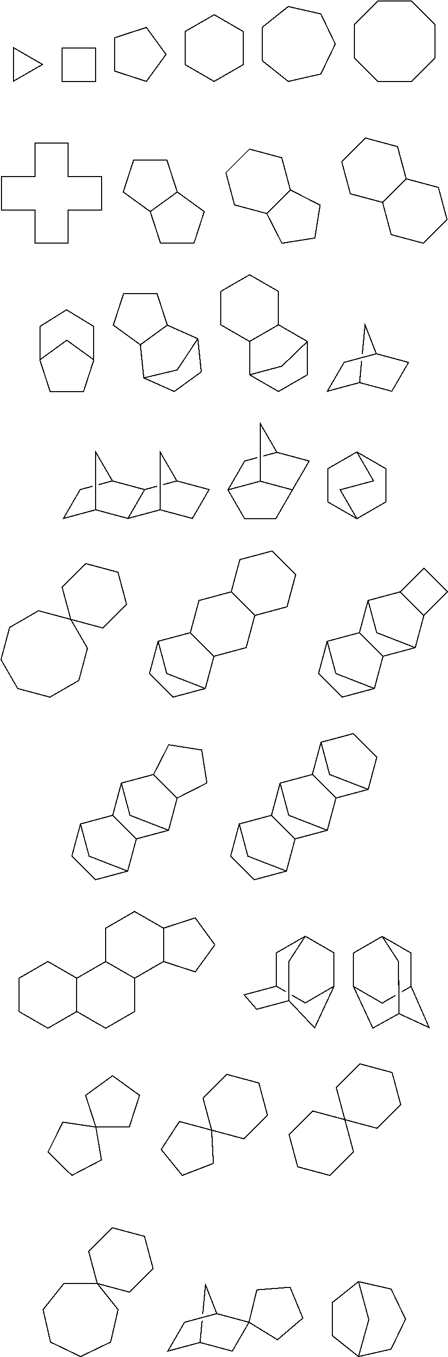

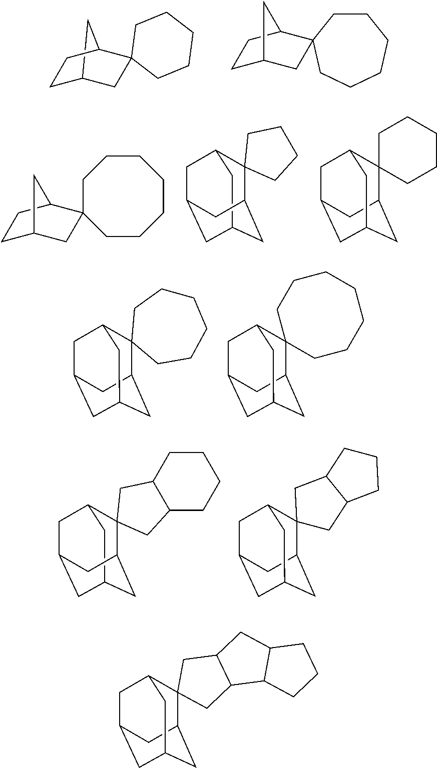

In a case where the alicyclic hydrocarbon group is a monocyclic type of cycloalkyl group, the number of carbon atoms of the alicyclic hydrocarbon group is preferably 3 to 24, is more preferably 3 to 20, is further preferably 3 to 18, is far more preferably 3 to 12, is still further preferably 3 to 10, and is even more preferably 3 to 8. In a case where the alicyclic hydrocarbon group is a polycyclic type of cycloalkyl group, the number of carbon atoms of the alicyclic hydrocarbon group is preferably 6 to 24, is more preferably 6 to 20, is further preferably 6 to 18, is far more preferably 6 to 12, and is still further preferably 7 to 12.

Examples of the aromatic hydrocarbon group include a group in which nb1 hydrogen atoms have been removed from an aryl group. Examples of the aryl group include a phenyl group, a naphthyl group, an anthryl group, a biphenyl group and a fluorenyl group. More specifically, examples thereof include aromatic hydrocarbon groups represented by the following formulae.

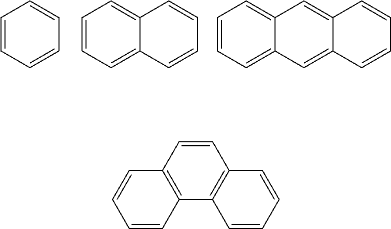

The number of carbon atoms of the aromatic hydrocarbon group is preferably 4 to 24, is more preferably 4 to 20, is further preferably 4 to 18, is far more preferably 5 to 14, is still further preferably 5 to 10, and even more preferably 6 to 10.

When —CH₂— contained in the hydrocarbon group for L^{b1} is replaced with —O—, —CO—, —S—, —SO— or —SO₂—, the number of carbon atoms before replacement corresponds to the defined number of carbon atoms in the hydrocarbon group.

Among the hydrocarbon groups for L^{b1}, examples of the group formed by replacing —CH₂— contained in the chain hydrocarbon group with —O—, —CO—, —S—, —SO—, or —SO₂— include a hydroxy group, a carboxy group, a thiol group, a carbonyl group, an oxy group, a thio group, a sulfonyl group, an alkoxy group, an alkylthio group, an alkylsulfonyl group, an alkoxycarbonyl group, an alkylcarbonyl group, an alkylcarbonyloxy group, an alkoxycarbonyloxy group, an alkoxyalkoxy group, an alkanediyloxy group, an alkanediyloxycarbonyl group, an alkanediylcarbonyl group, an alkanediylcarbonyloxy group, an alkanediyloxycarbonyloxy group, an alkanediylsulfonyl group, an alkanediylthio group, and a combined group of two or more of these groups. Examples of these replaced groups include the same groups as those exemplified in the present specification, within the acceptable range of the upper limit of the number of carbon atoms.

Among the hydrocarbon groups for L^{b1}, examples of the groups formed by replacing —CH₂— contained in an alicyclic hydrocarbon group, an aromatic hydrocarbon group, or a combined group of these groups with —O—, —CO—, —S—, —SO— or —SO₂— include groups containing a structure such as cyclic ether, cyclic ketone, cyclic ester (lactone), cyclic thioether, cyclic acetal or cyclic sulfonate (sultone), groups derived from a furan ring, a thiophene ring, and the like. Examples of these replaced groups include the same groups as those exemplified in the present specification, within the acceptable range of the upper limit of the number of carbon atoms.

Examples of the combined group of two or more groups selected from the group consisting of a chain hydrocarbon group, an alicyclic hydrocarbon group and an aromatic hydrocarbon group include a combined group of the above chain hydrocarbon group and the above alicyclic hydrocarbon group, a combined group of the above chain hydrocarbon group and the above aromatic hydrocarbon group, a combined group of the above alicyclic hydrocarbon group and the above aromatic hydrocarbon group, and a combined group of the above chain hydrocarbon group, the above alicyclic hydrocarbon group and the above aromatic hydrocarbon group. The combined group of an alicyclic hydrocarbon group and an aromatic hydrocarbon group may be a condensed ring.

In addition, examples of the group formed by replacing —CH₂— contained in the alicyclic hydrocarbon group or the combined group of the alicyclic hydrocarbon group and the aromatic hydrocarbon group with —O—, —CO—, —S—, —SO—, or —SO₂— include the same groups as those exemplified in the present specification, within the acceptable range of the upper limit of the number of carbon atoms.

Examples of the divalent hydrocarbon group for L^{b2} include a divalent chain hydrocarbon group, a divalent alicyclic hydrocarbon group, a divalent aromatic hydrocarbon group, and a combined group of two or more of these groups, and also include a group formed by removing one hydrogen atom from a monovalent hydrocarbon group, the formed group being bonded to Y^{b1}.

Examples of a divalent chain hydrocarbon group, a divalent alicyclic hydrocarbon group, a divalent aromatic hydrocarbon group and a divalent combined group of two or more of these groups, for L^{b2}, include groups formed by removing one hydrogen atom from a monovalent chain hydrocarbon group, a monovalent alicyclic hydrocarbon group, a monovalent aromatic hydrocarbon group and a monovalent combined group of two or more of these groups, respectively, which have been exemplified in L^{b1}, within the acceptable range of the upper limit of the number of carbon atoms.

Examples of substituents that the hydrocarbon groups for L^{b1} and L^{b2} optionally have include a halogen atom, a cyano group, and a nitro group. Examples of the halogen atom include a fluorine atom, a chlorine atom, a bromine atom and an iodine atom.

When L^{b1} and L^{b2} are combined groups of an alicyclic hydrocarbon group or an aromatic hydrocarbon group and a chain hydrocarbon group, it is acceptable to regard the chain hydrocarbon group substantially as a substituent which the alicyclic hydrocarbon group or the aromatic hydrocarbon group has. In addition, when —CH₂— of the chain hydrocarbon group contained in the hydrocarbon group of L^{b1} and L^{b2} is replaced with —O—, —CO—, —S—, —SO— or —SO₂—, then the hydrocarbon groups of L^{b1} and L^{b2} can substantially have a substituent such as a hydroxy group, a carboxy group, a carbonyl group, an oxycarbonyl group, a carbonyloxy group, an alkoxy group, an alkylcarbonyl group, an alkoxycarbonyl group, an alkylcarbonyloxy group, a thiol group, an alkylthio group, or an alkylsulfonyl group.

The number of substituents that the L^{b1} and the L^{b2} optionally have is not particularly limited, and L^{b1} and L^{b2} optionally have a plurality of substituents.

Examples of the cyclic hydrocarbon group for Y^{b1} include an alicyclic hydrocarbon group, an aromatic hydrocarbon group, and a combined group of an alicyclic hydrocarbon group and an aromatic hydrocarbon group.

Examples of the alicyclic hydrocarbon group and the aromatic hydrocarbon group for Y^{b1} include the same alicyclic hydrocarbon group and aromatic hydrocarbon group as those exemplified in L^{b1}, respectively, and when having no substituent, the groups for Y^{b1} may be a monovalent alicyclic hydrocarbon group and a monovalent aromatic hydrocarbon group.

Examples of substituents that the methyl group and the cyclic hydrocarbon group for Y^{b1} optionally have include a halogen atom, a cyano group, a nitro group, and a hydrocarbon group having 1 to 18 carbon atoms and optionally having a halogen atom, a cyano group or a nitro group (—CH₂— contained in the hydrocarbon group is optionally replaced with —O—, —S—, —CO—, —SO— or —SO₂—). The number of carbon atoms in the hydrocarbon group that the methyl group and the cyclic hydrocarbon group for Y^{b1} optionally have is not included in the number of carbon atoms of the methyl group and cyclic hydrocarbon group for Y^{b1}.

Examples of the halogen atom include the same halogen atoms as the halogen atoms which have been exemplified as the substituents for L^{b1} and L^{b2}.

Examples of the hydrocarbon group having 1 to 18 carbon atoms, which the methyl group and the cyclic hydrocarbon group for Y^{b1} optionally have as a substituent, include a chain hydrocarbon group, an alicyclic hydrocarbon group, an aromatic hydrocarbon group, and a combined group of these groups. Examples of the chain hydrocarbon group, the alicyclic hydrocarbon group, the aromatic hydrocarbon group and the combined group of two or more of these groups include the same groups as the chain hydrocarbon group, the alicyclic hydrocarbon group, the aromatic hydrocarbon group, and the combined group of two or more of these groups, which have been exemplified in L^{b1}, within the acceptable range of the upper limit of the number of carbon atoms.

In addition, examples of the group formed by replacing —CH₂— contained in the hydrocarbon group having 1 to 18 carbon atoms, which the methyl group and the cyclic hydrocarbon group for Y^{b1} optionally have as a substituent, with —O—, —S—, —CO—, —SO— or —SO₂— include the same groups as those exemplified as the group formed by replacing —CH₂— contained in the hydrocarbon group for L^{b1} with —O—, —S—, —CO—, —SO— or —SO₂—, within the acceptable range of the upper limit of the number of carbon atoms. The hydrocarbon group having 1 to 18 carbon atoms, which the methyl group and the cyclic hydrocarbon group for Y^{b1} optionally have as a substituent, may constitute a protecting group or a leaving group (acid labile group or base labile group), which is commonly used in the field.

It is preferable that the anion of the salts represented by formula (B1) is an anion represented by the following formula (B1-A1) (hereinafter referred to as “anion (B1-A1)”, in some cases), or an anion represented by formula (B1-A2) (hereinafter referred to as “anion (B1-A2)”, in some cases):

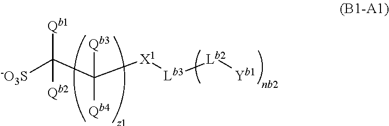

wherein in formula (B1-A1)
L^{b2} and Y^{b1} have the same meaning as L^{b2} and Y^{b1} in formula (B1);
Q^{b1}, Q^{b2}, Q^{b3} and Q^{b4} each independently represent a hydrogen atom, a fluorine atom, a perfluoroalkyl group having 1 to 6 carbon atoms, or an alkyl group having 1 to 6 carbon atoms;
z1 represents an integer of 0 to 6; when z1 is 2 or larger, a plurality of groups in the parentheses may be the same or different from each other;
X¹ represents —O—CO—, —CO—O—, —O—CO—O— or —O—;
L^{b3} represents a single bond or a hydrocarbon group having a valence of (nb2+1), having 1 to 24 carbon atoms, and optionally having a substituent, and —CH₂— contained in the hydrocarbon group is optionally replaced with —O—, —S—, —CO—, —SO— or —SO₂—;
nb2 represents an integer of 1 to 3; and when nb2 is 2 or larger, a plurality of groups in the parentheses may be the same or different from each other.

Examples of the alkyl group for Q^{b1}, Q^{b2}, Q^{b3} and Q^{b4} include a methyl group, an ethyl group, a propyl group, an isopropyl group, a butyl group, a sec-butyl group, a tert-butyl group, a pentyl group and a hexyl group.

Examples of the perfluoroalkyl group for Q^{b1}, Q^{b2}, Q^{b3} and Q^{b4} include a trifluoromethyl group, a perfluoroethyl group, a perfluoropropyl group, a perfluoroisopropyl group, a perfluorobutyl group, a perfluorosec-butyl group, a perfluorotert-butyl group, a perfluoropentyl group and a perfluorohexyl group.

Q^{b1} and Q^{b2} preferably contain a fluorine atom or a perfluoroalkyl group in at least one of the Q^{b1} and the Q^{b2}; are more preferably a fluorine atom or a perfluoroalkyl group; are further preferably a fluorine atom or a trifluoromethyl group; and are both even more preferably fluorine atoms.

Q^{b3} and Q^{b4} are each independently preferably a hydrogen atom, a fluorine atom, or a perfluoroalkyl group having 1 to 3 carbon atoms; Q^{b3} is preferably a hydrogen atom, a fluorine atom, or a perfluoroalkyl group having 1 to 3 carbon atoms; and Q^{b4} is preferably a hydrogen atom or a fluorine atom.
z1 is preferably an integer of 0 to 3, and is more preferably 0, 1 or 2.
X¹ is preferably —O—CO— or —CO—O—.

Examples of the hydrocarbon group for L^{b3} include the same hydrocarbon groups as those exemplified for L^{b1} in formula (B1), within the acceptable range of the upper limit of the number of carbon atoms.

L^{b3} is preferably a single bond, or a chain hydrocarbon group having 1 to 12 carbon atoms and optionally having a substituent (—CH₂— contained in the chain hydrocarbon group is optionally replaced with —O— or —CO—), an alicyclic hydrocarbon group having 3 to 18 carbon atoms and optionally having a substituent (—CH₂— contained in the alicyclic hydrocarbon group is optionally replaced with —O—, —S—, —CO—, —SO— or —SO₂—), an aromatic hydrocarbon group having 6 to 10 carbon atoms and optionally having a substituent (—CH₂— contained in the aromatic hydrocarbon group is optionally replaced with —O— or —S—), or a combined group of two or more of these groups; and is more preferably a single bond, a chain hydrocarbon group having 1 to 6 carbon atoms, or a group represented by the following formula (Lb3-1). In addition, when —CH₂— contained in the chain hydrocarbon group is replaced with —O— or —CO—, the number of —CH₂— contained in the chain hydrocarbon group replaced with —O— or —CO— is preferably 1 to 4; and it is preferable that one —CH₂—CH₂— contained in the chain hydrocarbon group is replaced with —O—CO— or —CO—O—, or one —CH₂—CH₂—CH₂— contained in the chain hydrocarbon group is replaced with —O—CO—O—:

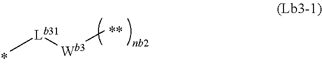

wherein in formula (Lb3-1)
nb2 has the same meaning as nb2 in formula (B1-A1);
L^{b31} represents a single bond or a chain hydrocarbon group having 1 to 12 carbon atoms, —CH₂— contained in the chain hydrocarbon group is optionally replaced with —O— or —CO—, and the chain hydrocarbon group optionally has a substituent;
W^{b3} represents an alicyclic hydrocarbon group having 3 to 18 carbon atoms, or an aromatic hydrocarbon group having 6 to 10 carbon atoms; —CH₂— contained in the alicyclic hydrocarbon group is optionally replaced with —O—, —S—, —CO—, —SO— or —SO₂—, —CH₂— contained in the aromatic hydrocarbon group is optionally replaced with —O— or —S—, and the alicyclic hydrocarbon group and the aromatic hydrocarbon group optionally have a substituent; and
* and ** represent bonding sites, and * represents a bonding site to X¹.

In formula (Lb3-1), examples of the chain hydrocarbon group for L^{b31} include the same chain hydrocarbon groups as those exemplified in L^{b1}, as long as the upper limit of the number of carbon atoms allows.

In formula (Lb3-1), examples of the alicyclic hydrocarbon group and aromatic hydrocarbon group for W^{b3} may be the same as the alicyclic hydrocarbon group and aromatic hydrocarbon group exemplified in L^{b1}, as long as the upper limit of the number of carbon atoms allows.

In formula (Lb3-1), examples of the substituent that the chain hydrocarbon group for L^{b31} optionally has and the substituent that the alicyclic hydrocarbon group and the aromatic hydrocarbon group for W^{b3} optionally have include the same groups as the substituents exemplified as the substituents that the hydrocarbon group for L^{b1} optionally has.

It is preferable that L^{b31} is a single bond or an alkanediyl group having 1 to 6 carbon atoms (—CH₂— contained in the alkanediyl group is optionally replaced with —O— or —CO—).

Examples of the alicyclic hydrocarbon group and aromatic hydrocarbon group for W^{b3} include the following alicyclic hydrocarbon groups and aromatic hydrocarbon groups. In the following alicyclic hydrocarbon groups and aromatic hydrocarbon groups, * and ** represent bonding sites, * represents a bonding site to X¹ or L^{b31}, ** represents a hydrogen atom, a substituent or a bonding site to L^{b2}, and at least one ** represents a bonding site to L^{b2}. In addition, in the following alicyclic hydrocarbon groups, —CH₂— contained in the alicyclic hydrocarbon group is optionally replaced with —O—, —S—, —CO—, —SO—, or —SO₂—. When —CH₂— contained in the alicyclic hydrocarbon group is replaced with —O—, —S—, —CO—, —SO— or —SO₂—, an ether ring, an ester ring (lactone ring), a carbonic acid ester ring, a sulfonic acid ester ring (sultone ring) or an acetal ring is preferably formed.

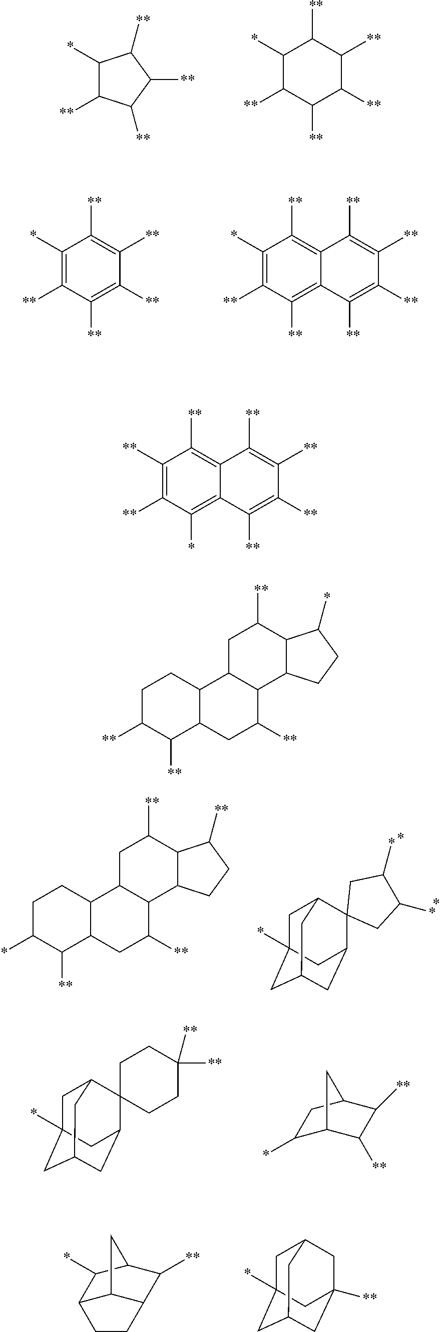

In formula (B1-A1), L^{b2} is preferably a single bond or a chain hydrocarbon group having 1 to 12 carbon atoms (where —CH₂— included in the chain hydrocarbon group is optionally replaced with —O— or —CO—), more preferably a single bond, —O—, —O—CO—, —CO—O—, —O—CO—O—, or *-L^{b21}-X²-L^{b22}-** (where one of L^{b21} and L^{b22} represents a chain hydrocarbon group having 1 to 6 carbon atoms, and the other represents a single bond or a chain hydrocarbon group having 1 to 6 carbon atoms; X² represents —O—, —CO—O—, —O—CO—, or —O—CO—O—; * and ** represent bonding sites; and ** represents the bonding site to Y^{b1}; provided that the total number of carbon atoms in L^{b21}, X², and L^{b22} is 12 or less.).

In formula (B1-A1), Y^{b1} is preferably a cyclic hydrocarbon group having 3 to 20 carbon atoms and optionally having a substituent (—CH₂— contained in the cyclic hydrocarbon group is optionally replaced with —O—, —CO—, —S—, —SO— or —SO₂—), is more preferably an alicyclic hydrocarbon group having 3 to 18 carbon atoms and optionally having a substituent (—CH₂— contained in the alicyclic hydrocarbon group is optionally replaced with —O—, —CO—, —S—, —SO— or —SO₂—), or an aromatic hydrocarbon group having 6 to 18 carbon atoms and optionally having a substituent, and is further preferably an alicyclic hydrocarbon group having 3 to 16 carbon atoms and optionally having a substituent (—CH₂— contained in the alicyclic hydrocarbon group is optionally replaced with —O—, —CO—, —S—, —SO— or —SO₂—), or an aromatic hydrocarbon group having 6 to 10 carbon atoms and optionally having a substituent. Specifically, the groups represented by the following formula (Y1) to formula (Y36) are more preferable. In formula (Y1) to formula (Y36), R^{Yb} represents an alkyl group having 1 to 4 carbon atoms among a hydrogen atom or a substituent that the cyclic hydrocarbon group for Y^{b1} optionally has, R^{Yc} represents a hydrogen atom or a substituent that the cyclic hydrocarbon group for Y^{b1} optionally has, and * represents a bonding site to L^{b2}. The alicyclic hydrocarbon group and the aromatic hydrocarbon group represented by the following formulae optionally have another substituent which is not particularly shown in the following formulae, though.

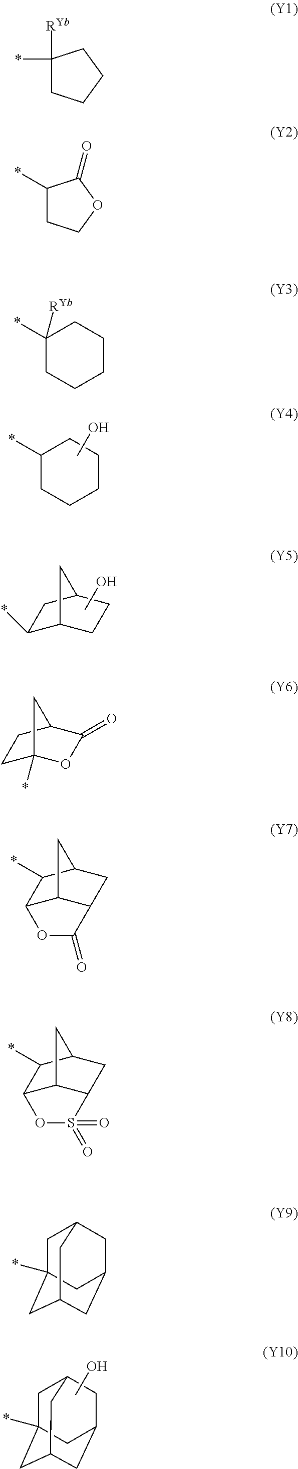

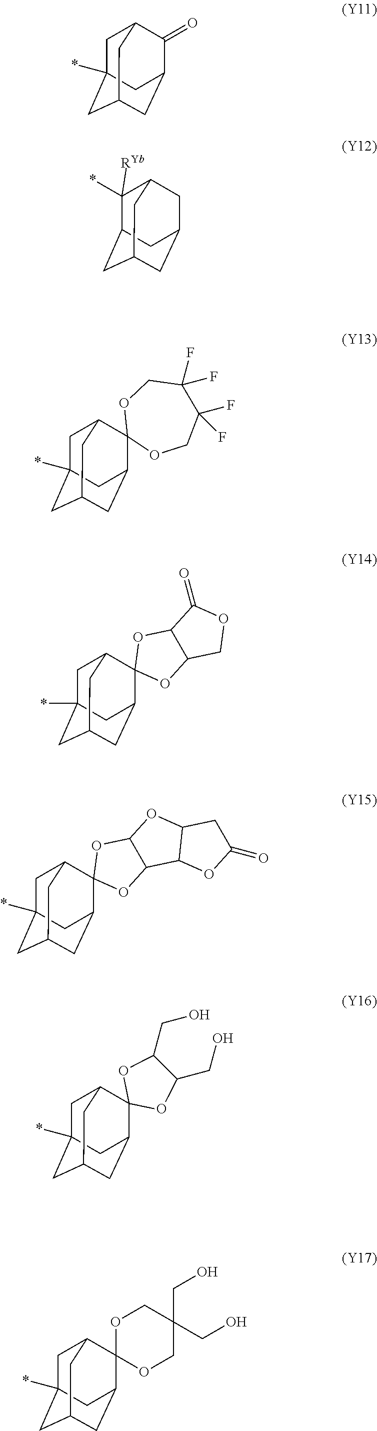

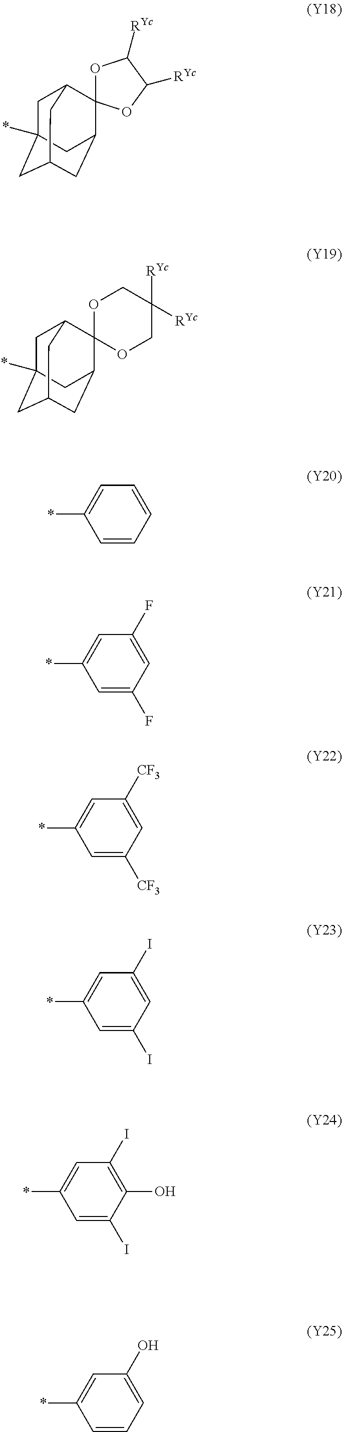

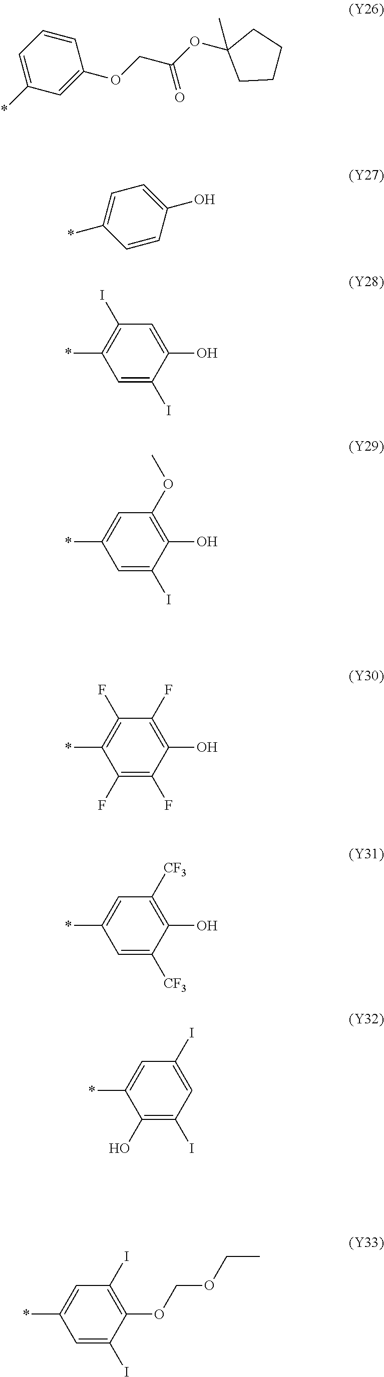

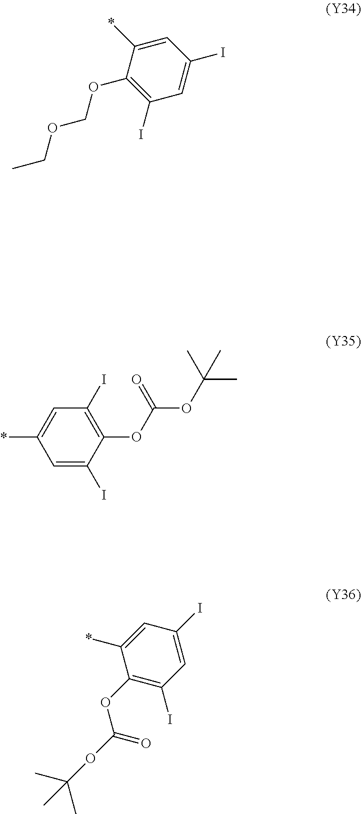

The anion represented by the formula (B1-A1) is preferably anions represented by formula (B1-A1-1) to formula (B1-A1-85) (hereinafter, referred to as “anion (B1-A1-1)” or the like according to the formula number, in some cases), and is more preferably anions represented by any of formula (B1-A1-1) to formula (B1-A1-4), formula (B1-A1-9), formula (B1-A1-10), formula (B1-A1-24) to formula (B1-A1-33), formula (B1-A1-36) to formula (B1-A1-40), and formula (B1-A1-47) to formula (B1-A1-85).

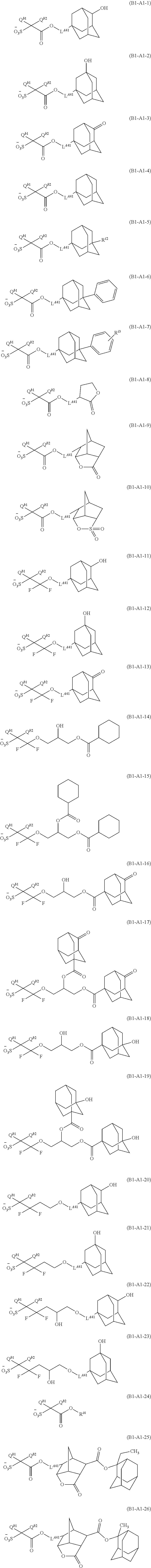

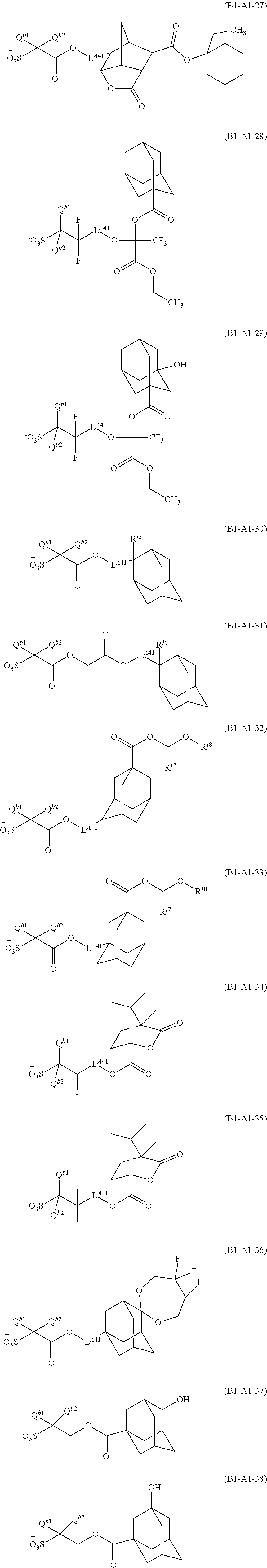

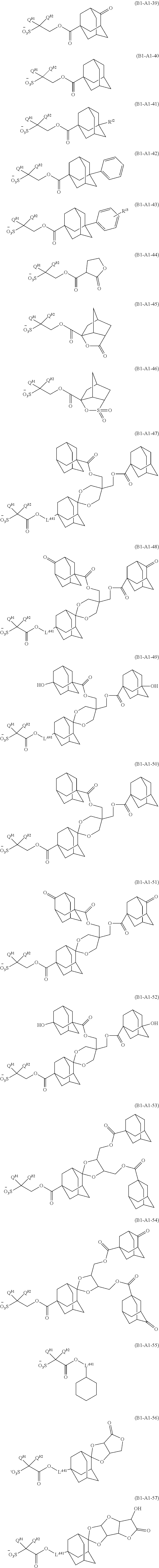

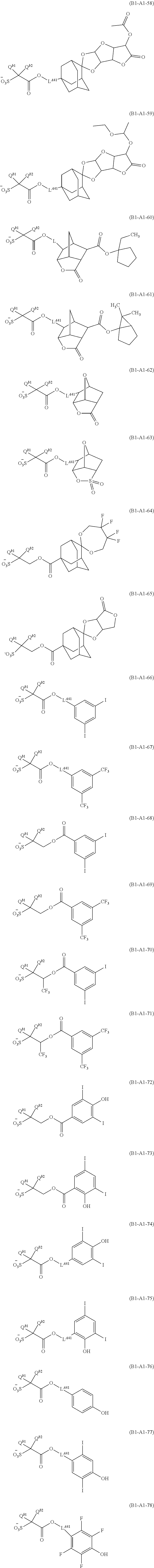

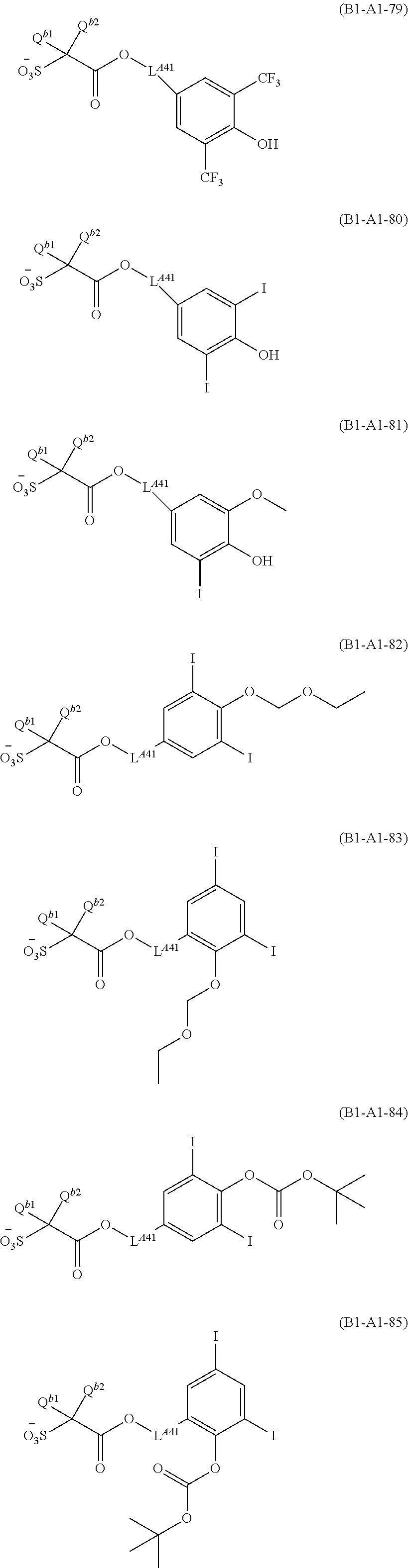

Here, Rⁱ² to Rⁱ⁷ are each independently an alkyl group having 1 to 4 carbon atoms, for example, and is preferably a methyl group or an ethyl group. Rⁱ⁸ is, for example, an aliphatic hydrocarbon group having 1 to 12 carbon atoms, is preferably an alkyl group having 1 to 4 carbon atoms, an alicyclic hydrocarbon group having 5 to 12 carbon atoms, or a group formed by combining these groups, and is more preferably a methyl group, an ethyl group, a cyclohexyl group or an adamantyl group. L^{A41} is a single bond or an alkanediyl group having 1 to 4 carbon atoms.

Q^{b1} and Q^{b2} have the same meanings as above.

Specific examples of the anion represented by formula (B1-A1) include anions described in Japanese Patent Laid-Open No. 2010-204646.

Preferable examples of the anion represented by formula (B1-A1) include anions represented by formula (B1a-1) to formula (B1a-82), respectively. Among the anions, an anion is preferable which is represented by any one of formula (B1a-1) to formula (B1a-4), formula (B1a-7) to formula (B1a-11), formula (B1a-14) to formula (B1a-30), and formula (B1a-35) to formula (B1a-72).

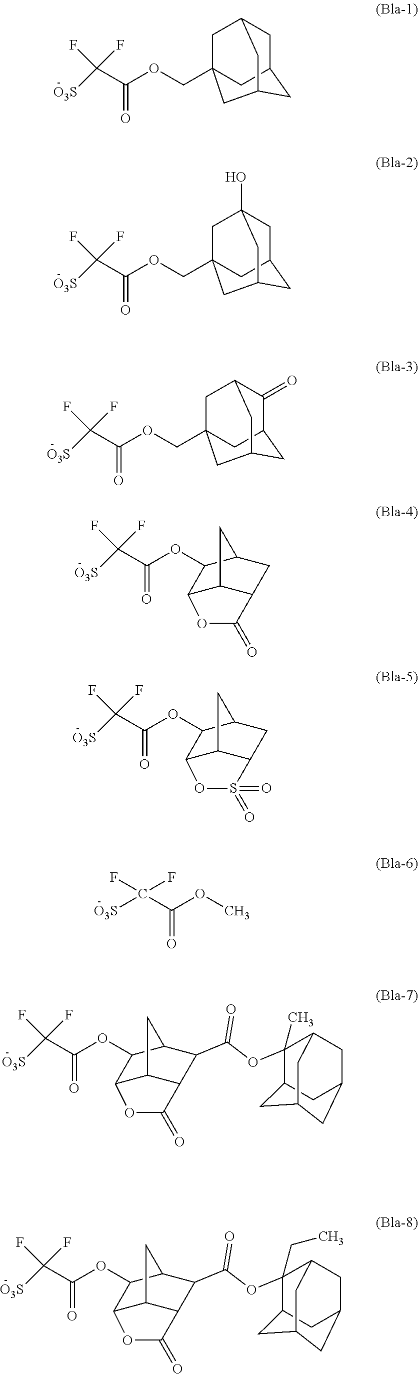

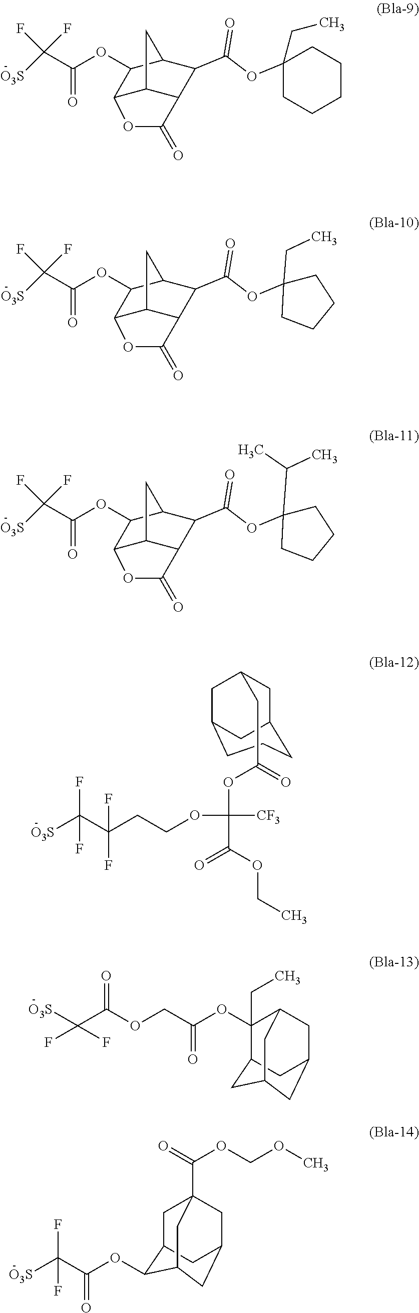

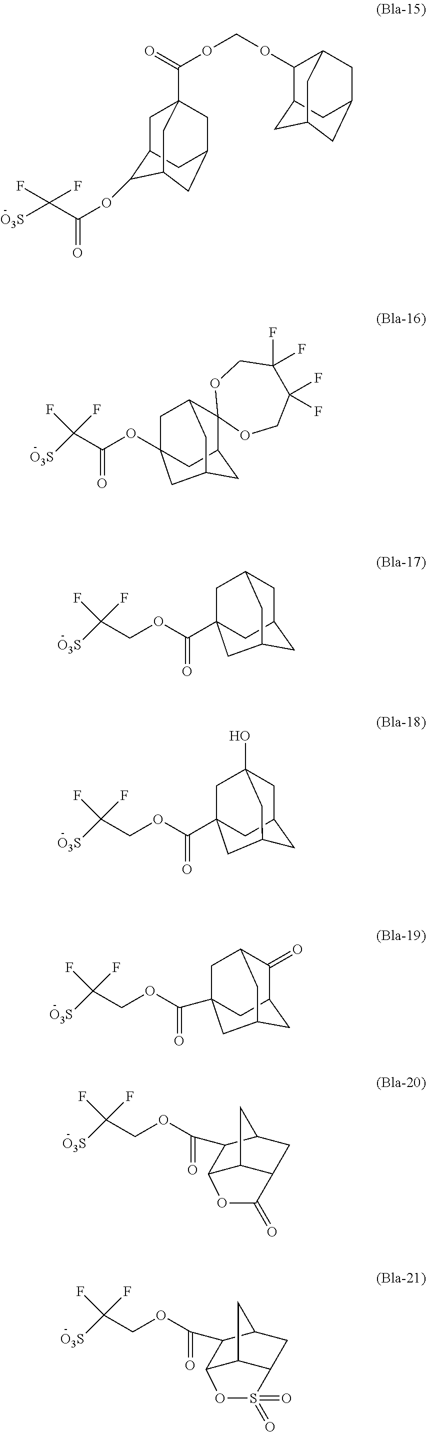

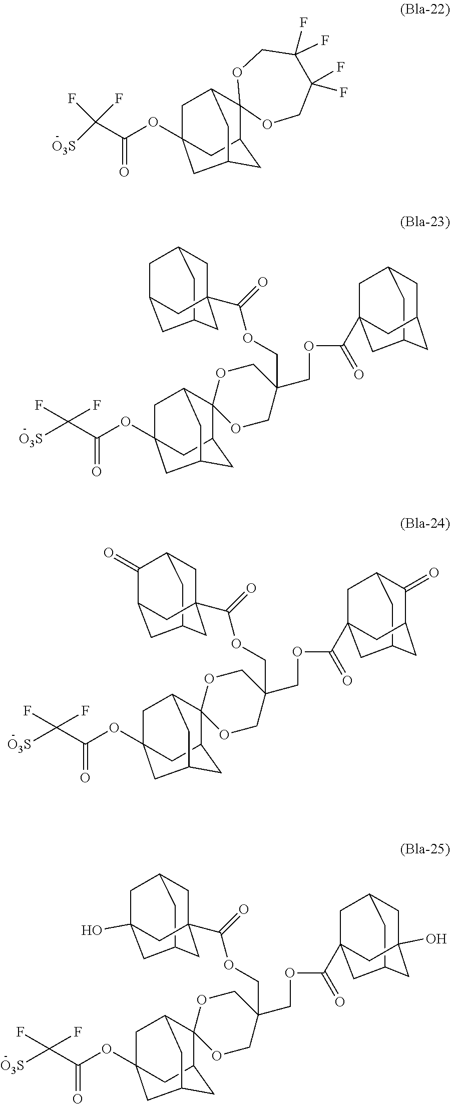

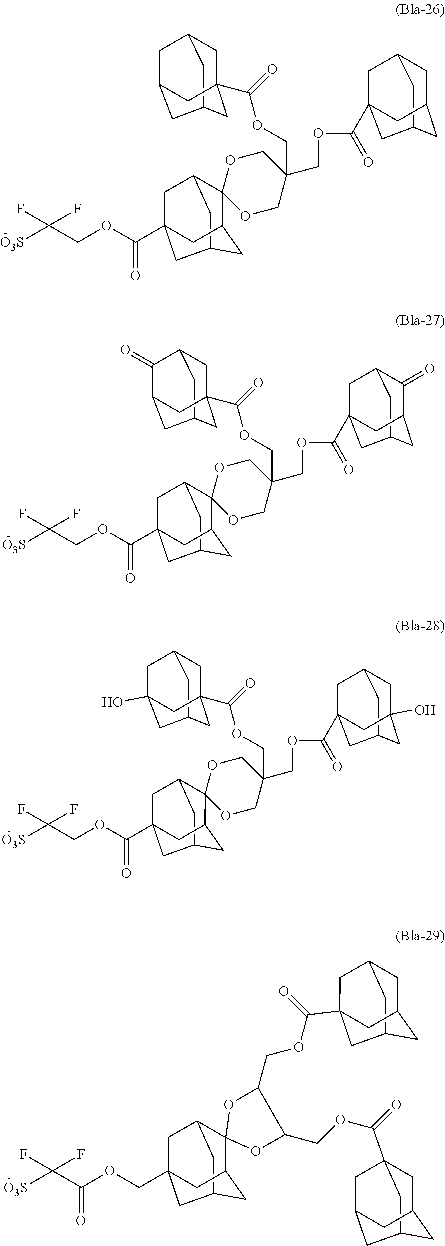

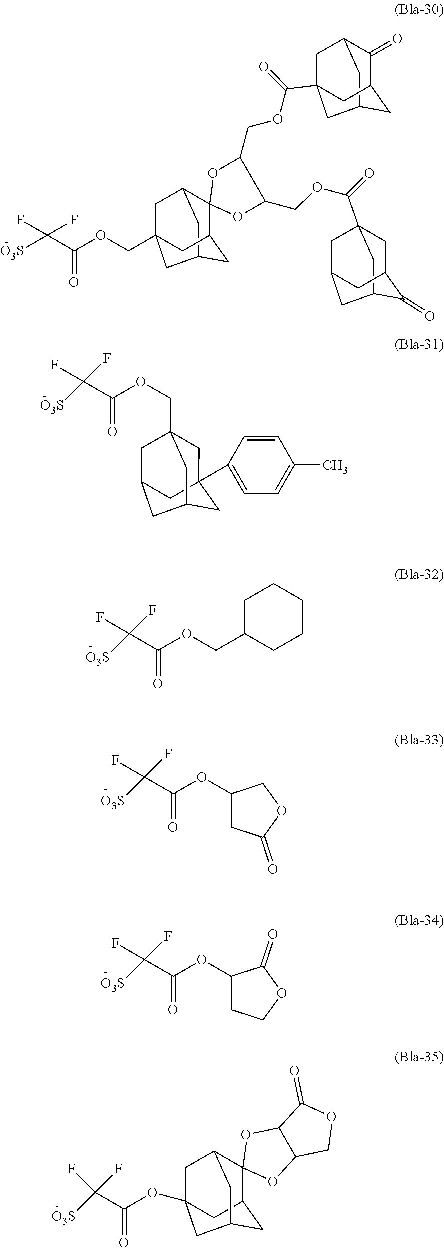

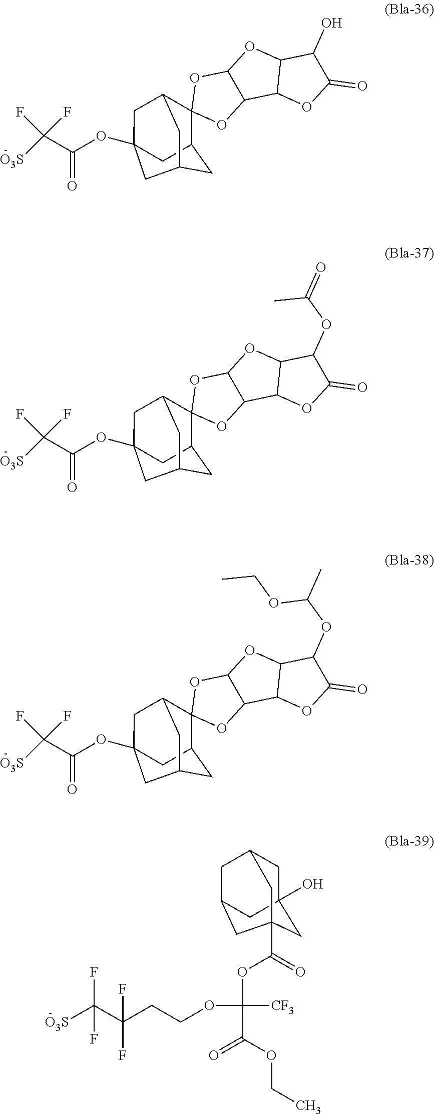

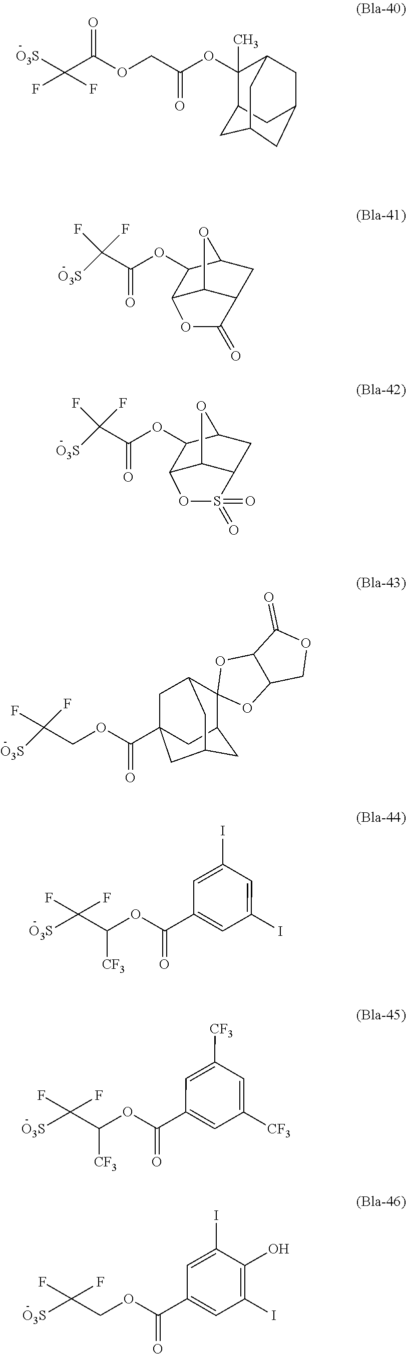

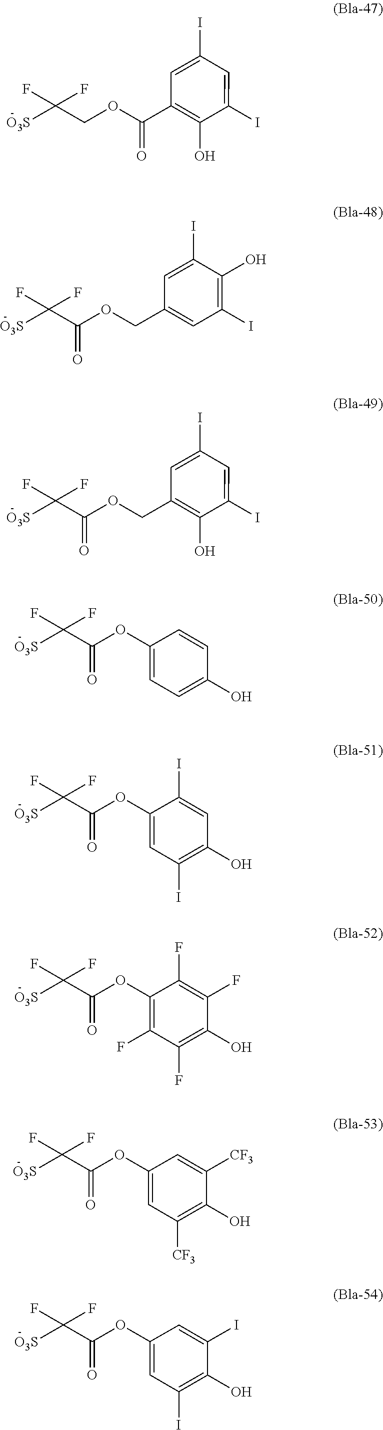

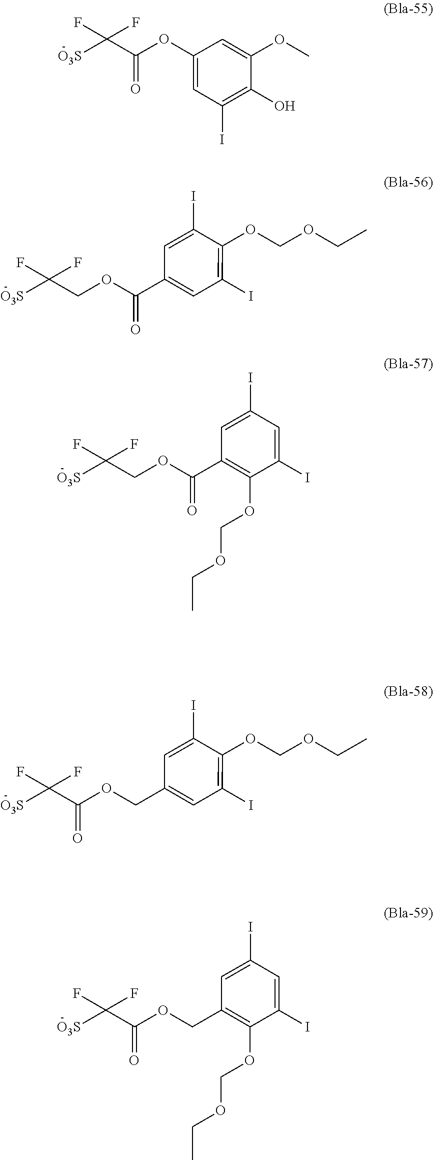

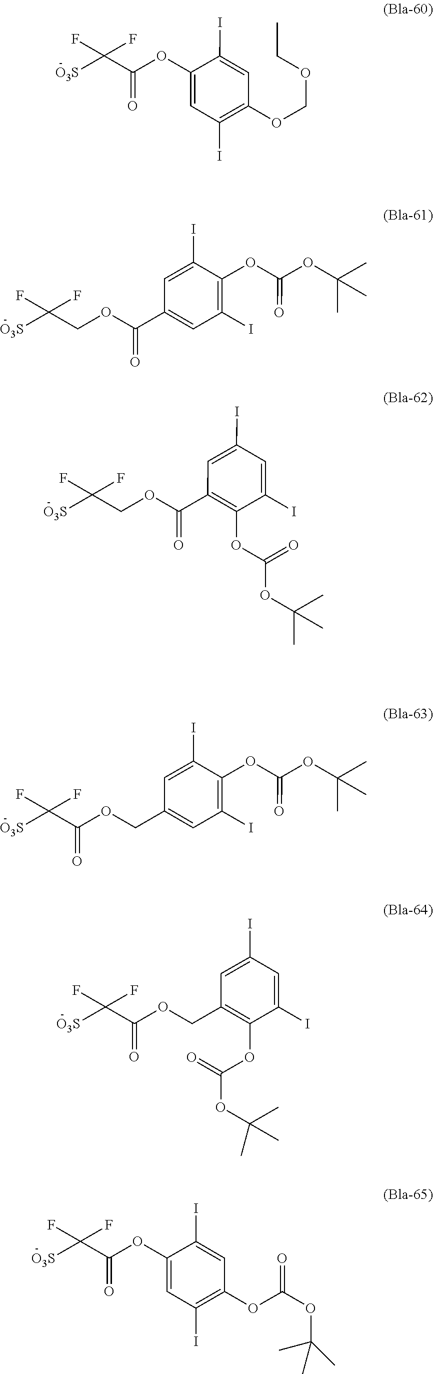

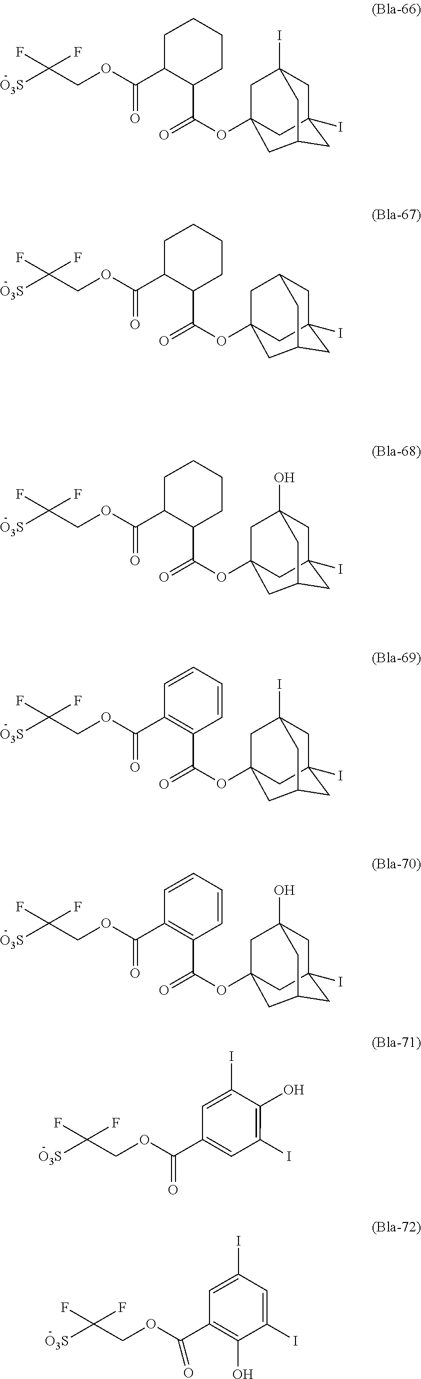

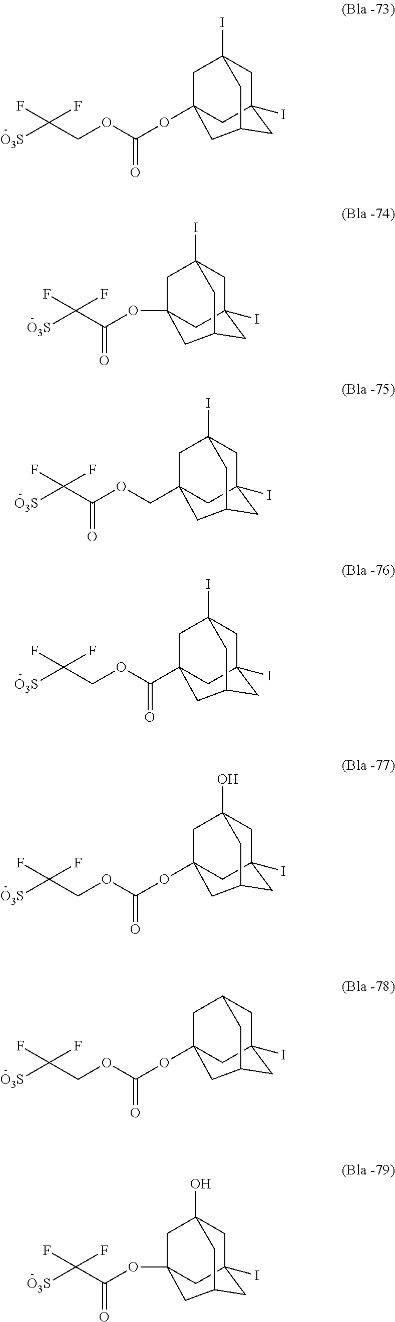

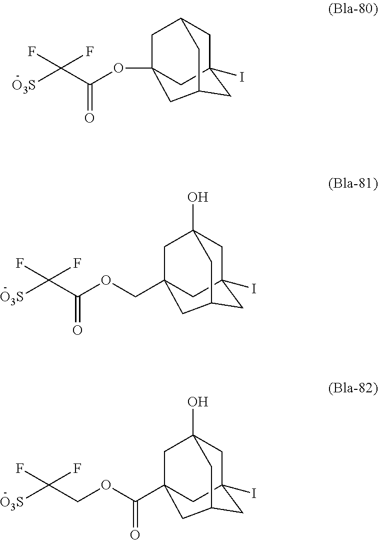

The anion represented by formula (B1-A2) is represented by the following formula.

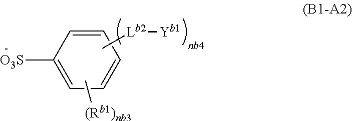

wherein in formula (B1-A2),
L^{b2} and Y^{b1} have the same meaning as L^{b2} and Y^{b1} in formula (B1);
R^{b1} represents a halogen atom or an alkyl group having 1 to 6 carbon atoms, and —CH₂— contained in the alkyl group is optionally replaced with —O— or —CO—;
nb4 represents an integer of 1 to 5; when nb4 is 2 or larger, a plurality of groups in the parentheses may be the same or different from each other;
nb3 represents an integer of 0 to 4; when nb3 is 2 or more, a plurality of Rb may be the same or different from each other; and
but 1≤nb4+nb3≤5 is satisfied.

In formula (B1-A2), examples of the hydrocarbon group for L^{b2} and examples of the cyclic hydrocarbon group for Y^{b1} include the same groups as in the examples of the hydrocarbon group for L^{b2} and the examples of the cyclic hydrocarbon group for Y^{b1} in the formula (B1), within the acceptable range of the upper limit of the number of carbon atoms. Examples of the substituents that the hydrocarbon group for L^{b2}, the methyl group and the cyclic hydrocarbon group for Y^{b1} optionally have are also the same as those of the substituents that the hydrocarbon group for L^{b2}, the methyl group and the cyclic hydrocarbon group for Y^{b1} in formula (B1) optionally have.

These hydrocarbon groups, methyl groups and cyclic hydrocarbon groups optionally have one substituent or a plurality of substituents.

L^{b2} is preferably a chain hydrocarbon group having 1 to 12 carbon atoms (where —CH₂— contained in the chain hydrocarbon group is optionally replaced with —O— or —CO—), is more preferably *—CO—O-L^{b41}- (where L^{b41} is a single bond, or a chain hydrocarbon group having 1 to 6 carbon atoms, and —CH₂— contained in the chain hydrocarbon group is optionally replaced with —O— or —CO—; * represents a bonding site to a benzene ring to which SO₃⁻ is bonded; and L^{b41} is preferably a single bond, or a chain hydrocarbon group having 1 to 3 carbon atoms), and is further preferably *—CO—O—.

Y^{b1} is preferably a cyclic hydrocarbon group having 3 to 20 carbon atoms and optionally having a substituent (—CH₂— contained in the cyclic hydrocarbon group is optionally replaced with —O—, —CO—, —S— or —SO₂—), is more preferably an alicyclic hydrocarbon group having 3 to 18 carbon atoms and optionally having a substituent (—CH₂— contained in the alicyclic hydrocarbon group is optionally replaced with —O—, —CO—, —S— or —SO₂—), or an aromatic hydrocarbon group having 6 to 18 carbon atoms and optionally having a substituent, and is further preferably the alicyclic hydrocarbon group or aromatic hydrocarbon group exemplified in Y^{b1} and L^{b1} of formula (B1-A1). Specifically, it is preferably the groups represented by the above formula (Y1) to formula (Y36), and is more preferably the groups represented by the above formula (Y1) to formula (Y19).

The substituent that the cyclic hydrocarbon group for Y^{b1} optionally has is preferably a halogen atom, or an alkyl group having 1 to 6 carbon atoms (—CH₂— contained in the alkyl group is optionally replaced with —O—, —S—, —CO—, —SO— or —SO₂—), is more preferably a fluorine atom, an iodine atom, or an alkyl group having 1 to 4 carbon atoms (—CH₂— contained in the alkyl group is optionally replaced with —O—, —S—, —CO—, —SO— or —SO₂—), is further preferably a fluorine atom, an iodine atom, a hydroxy group, an alkoxy group having 1 to 3 carbon atoms, or an alkyl group having 1 to 4 carbon atoms, and is far more preferably a fluorine atom, an iodine atom, a hydroxy group, or an alkyl group having 1 to 4 carbon atoms. The substituents for Y^{b1} may constitute a protecting group or a leaving group (acid labile group or base labile group), which is commonly used in the field.

In formula (B1-A2), nb4 is preferably an integer of 1 to 4, is more preferably an integer of 1 to 3, is further preferably 1 or 2, and is even more preferably 2. When nb4 is 1 or 2, a bonding position of -L^{b2}-Y^{b1} is preferably the following structure which is the m-position substitution on the benzene ring with respect to the bonding position of SO₃⁻.

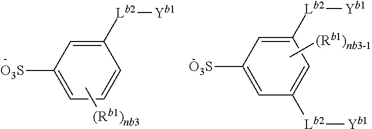

wherein L^{b2}, Y^{b1}, R^{b1} and nb3 have the same meanings as in formula (B1-A2).

In formula (B1-A2), in a case where nb4 is 2 or larger, it is preferable that a plurality of L^{b2} and Y^{b1} are each the same group.

Examples of the alkyl group having 1 to 6 carbon atoms for R^{b1} include alkyl groups such as a methyl group, an ethyl group, a propyl group, an isopropyl group, a butyl group, an isobutyl group, a tert-butyl group, a pentyl group and a hexyl group. The number of carbon atoms of the alkyl group is preferably 1 to 4, and is more preferably 1 to 3.

Examples of the halogen atom for R^{b1} include a fluorine atom, a chlorine atom, a bromine atom and an iodine atom.

Examples of the group formed by replacing —CH₂— contained in an alkyl group with —O— or —CO— include a hydroxy group, a carboxy group, an alkoxy group, an alkoxycarbonyl group, an alkylcarbonyl group, an alkylcarbonyloxy group, an oxy group and a carbonyl group. Examples of these replaced groups include the same groups as those exemplified in the present specification, within the acceptable range of the upper limit of the number of carbon atoms.

R^{b1} is preferably a halogen atom, or an alkyl group having 1 to 4 carbon atoms (where —CH₂— contained in the alkyl group is optionally replaced with —O— or —CO—), is more preferably a fluorine atom, an iodine atom, or an alkyl group having 1 to 3 carbon atoms (where —CH₂— contained in the alkyl group is optionally replaced with —O— or —CO—), and is further preferably a fluorine atom, an iodine atom, a hydroxy group, a methoxy group or a methyl group.

nb3 is an integer of 0 to 4 (that is, nb3-1 is an integer of 0 to 3), preferably an integer of 0 to 3, and more preferably an integer of 0 to 2. In one embodiment, nb3 is preferably 0. In another embodiment, nb3 is preferably 1 or 2. When nb3 is 1, R^{b1} is preferably a halogen atom, and R^{b1} is more preferably a fluorine atom or an iodine atom. When nb3 is 2, it is preferable that one of R^{b1} is a halogen atom and the other is a halogen atom or an alkyl group having 1 to 4 carbon atoms, and it is more preferable that one of R^{b1} is a fluorine atom or an iodine atom and the other is a fluorine atom, an iodine atom or an alkyl group having 1 to 3 carbon atoms.

Examples of the anion represented by formula (B1-A2) include the following anions. Among these anions, anions are preferable which are represented by formula (B2a-1) to formula (B2a-32), and anions are more preferable which are represented by formula (B2a-1) to formula (B2a-11) and formula (B2a-16) to formula (B2a-20). In the following anions, a part of R^{b1} is omitted, and the anions may contain substituents which are not illustrated.

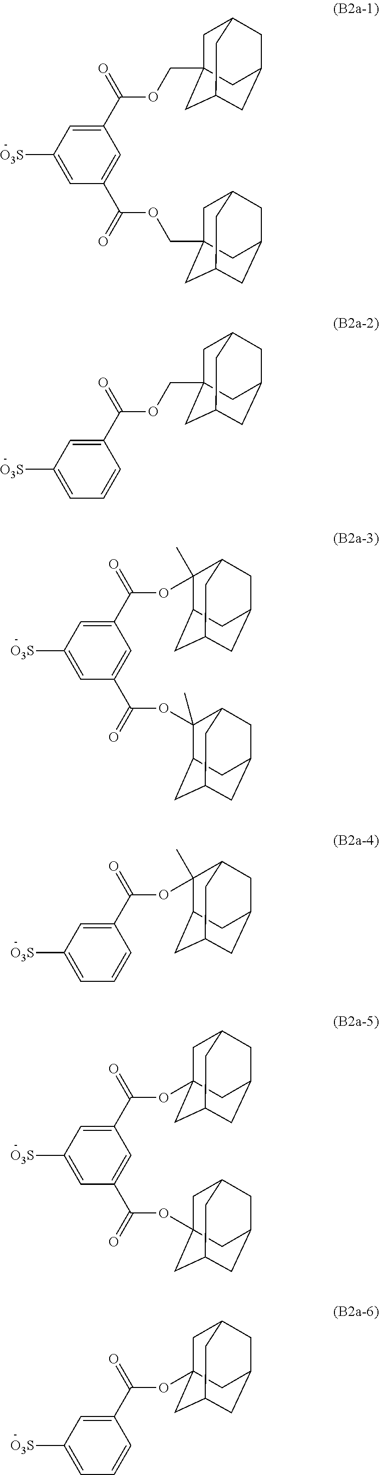

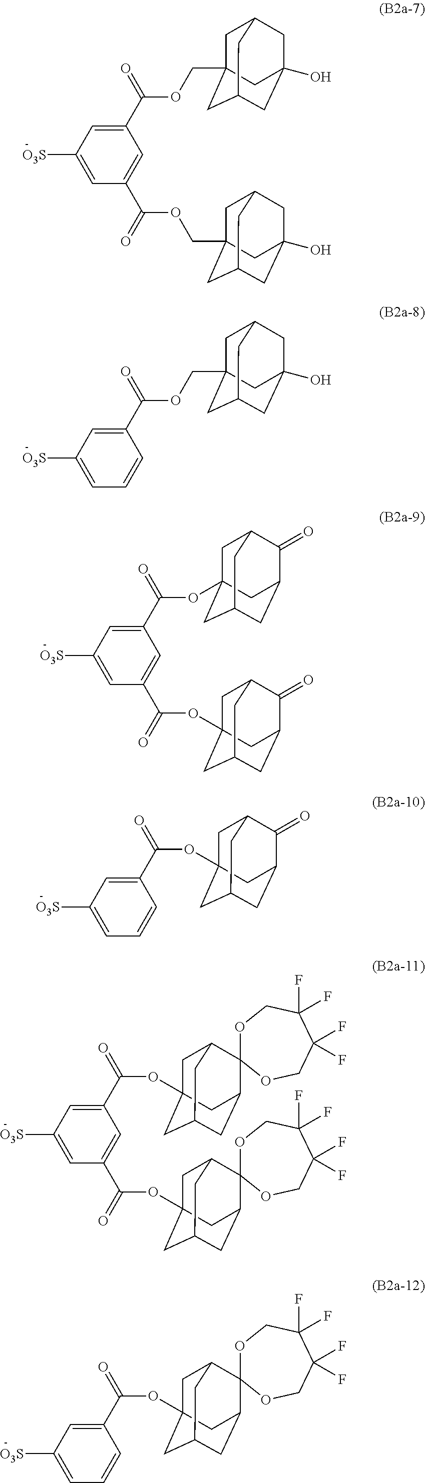

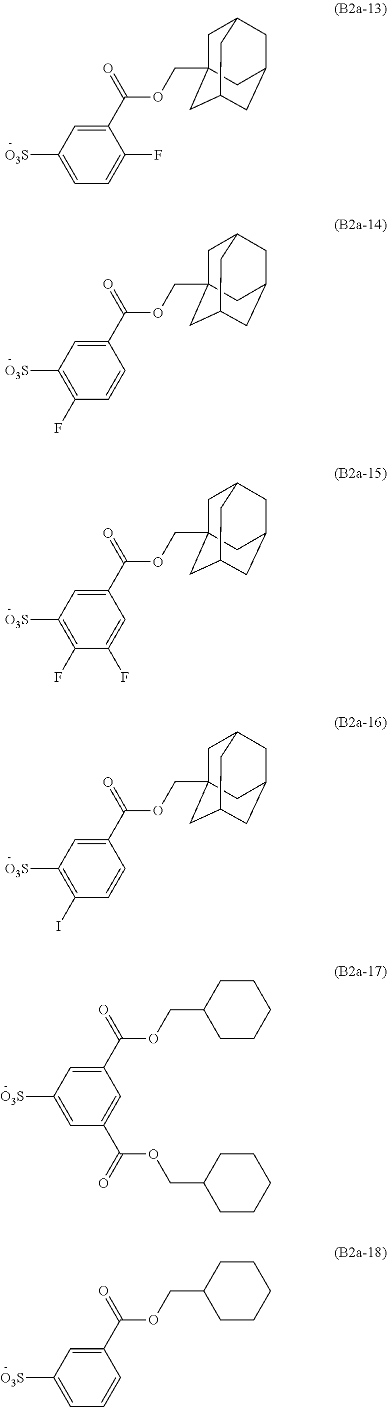

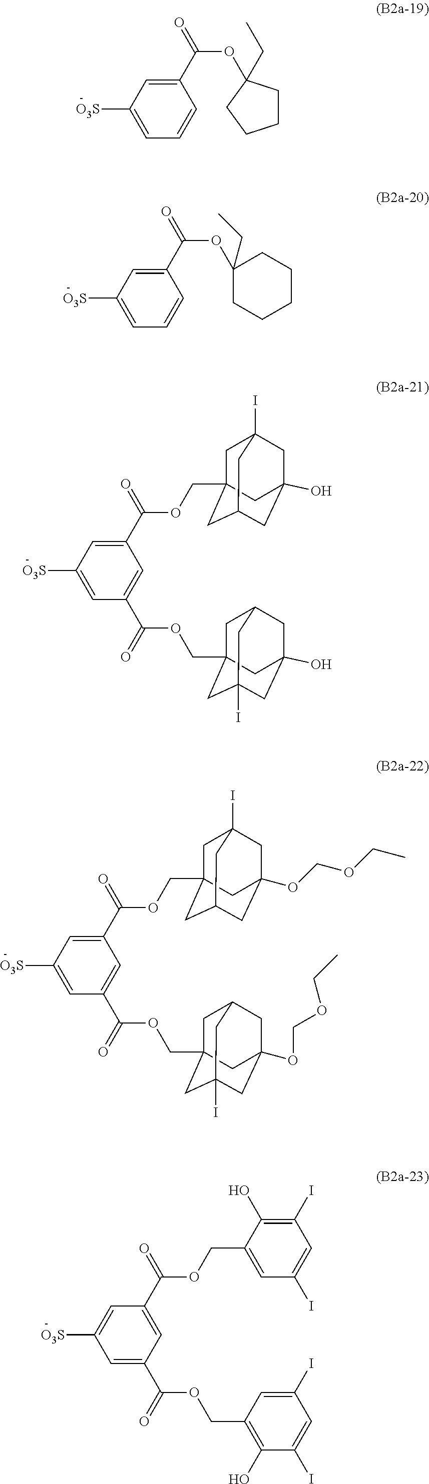

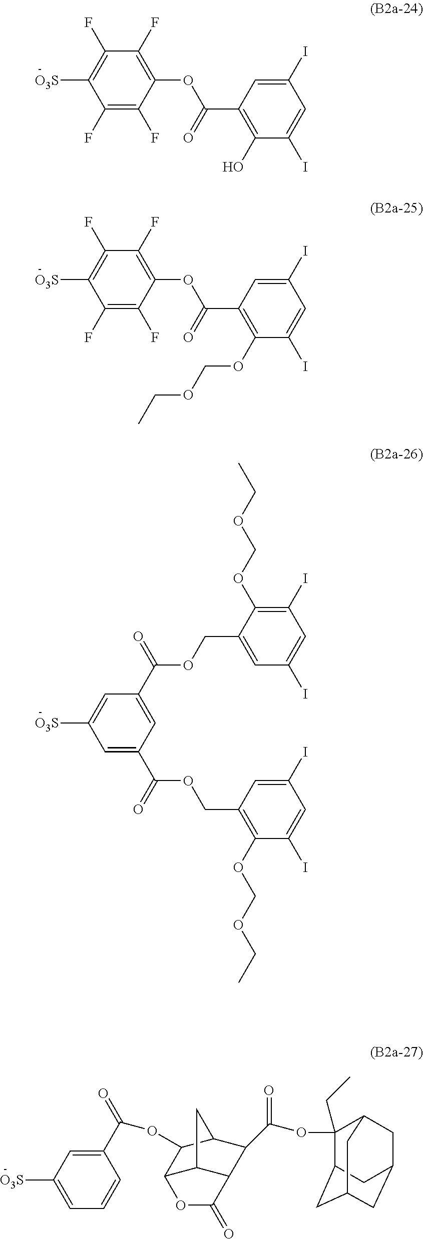

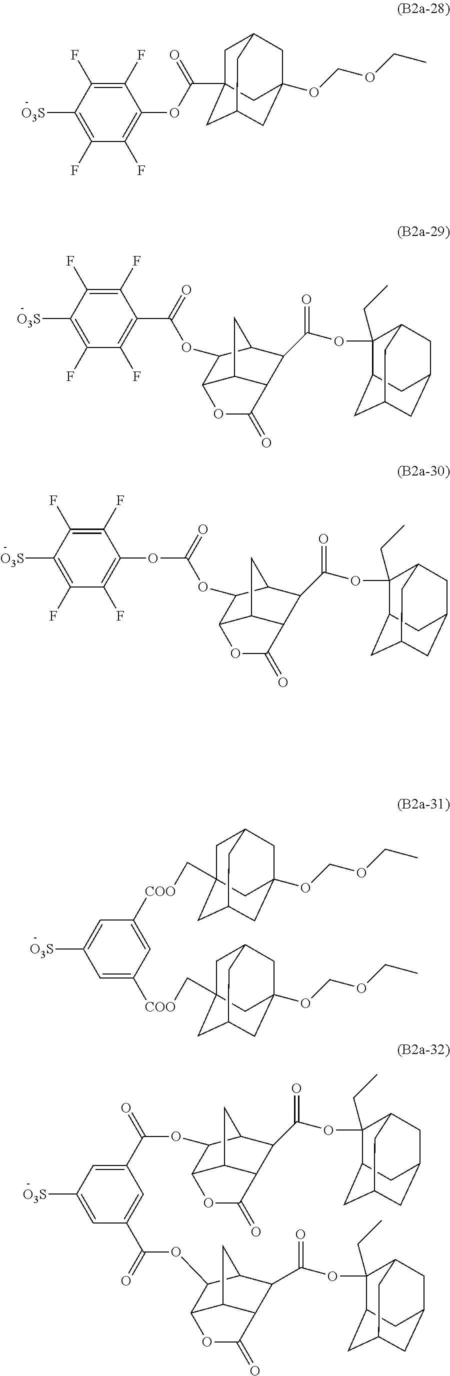

Examples of the acid generating agent (B2) include salts represented by the following formula:

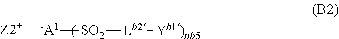

wherein in formula (B2),
A¹ represents a nitrogen atom or a carbon atom.
L^{b2′} represents a single bond, or a divalent hydrocarbon group having 1 to 24 carbon atoms and optionally having a substituent, and —CH₂— contained in the hydrocarbon group is optionally replaced with —O—, —S—, —CO—, —SO— or —SO₂—;
Y^{b1′} represents a methyl group optionally having a substituent, or a cyclic hydrocarbon group having 3 to 24 carbon atoms and optionally having a substituent, and —CH₂— contained in the cyclic hydrocarbon group is optionally replaced with —O—, —S—, —CO—, —SO— or —SO₂—;
nb5 represents an integer of 2 or 3; when nb5 is 2, A¹ is a nitrogen atom, and when nb5 is 3, A¹ is a carbon atom; the groups within a plurality of parentheses are the same or different from each other, and the groups in the two parentheses may be combined to form a ring that includes A′;
Z2⁺ represents an organic cation.

In formula (B2), examples of the hydrocarbon group, the cyclic hydrocarbon group, and the substituent for L^{b2′} and Y^{b1′} include the same groups as those exemplified for L^{b2} and Y^{b1} in formula (B1-A1).

L^{b2′} is preferably a single bond, *-L^{b23}-, *-L^{b23}-X²—, or *-L^{b23}-X²—W²—X³— (where L^{b23} represents a chain hydrocarbon group having 1 to 6 carbon atoms and optionally having a fluorine atom; X² and X³ independently represent —O—, —CO—O—, —O—CO—, —O—CO—O—, or —O—; W² represents an alicyclic hydrocarbon group having 3 to 12 carbon atoms, and the —CH₂— included in the alicyclic hydrocarbon group is optionally replaced with —O— or —CO—; and * represents a bonding site to SO₂.)

Y^{b1′} is preferably a methyl group having a fluorine atom, or a cyclic hydrocarbon group that has 3 to 20 carbon atoms and optionally have a substituent (where —CH₂— contained in the cyclic hydrocarbon group is optionally replaced with —O—, —CO—, —S—, —SO— or —SO₂—), and is more preferably a trifluoromethyl group, an alicyclic hydrocarbon group having 3 to 18 carbon atoms and optionally having a substituent (where —CH₂— contained in the alicyclic hydrocarbon group is optionally replaced with —O—, —S—, —CO—, —SO— or —SO₂—), or an aromatic hydrocarbon group having 6 to 18 carbon atoms and optionally having a substituent. Specifically, it is preferable to be a trifluoromethyl group, or a group represented by formula (Y1) to formula (Y36) exemplified in formula (B1-A1).

In the anion of the salt represented by formula (B2), when two —SO₂-L^{b2′}-Y^{b1′} are taken together to form a ring containing A¹, for example, an anion represented by formula (B2′) is exemplified:

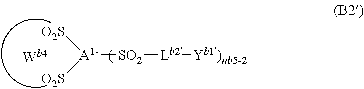

wherein in formula (B2′),
A¹, Y^{b1′}, L^{b2′} and nb5 have the same meanings as in formula (B2); and
W^{b4} represents a disulfonyl imide ring or a disulfonyl methide ring, each of which has 2 to 12 carbon atoms and optionally has a fluorine atom.

The disulfonyl imide ring or disulfonyl methide ring for W^{b4} preferably has 3 to 12 carbon atoms, more preferably has 3 to 6 carbon atoms, and the hydrogen atom of the methylene group contained in the ring is preferably replaced with a fluorine atom.

Examples of the anion of the salt represented by formula (B2) include the following. Among the anions, anions are preferable which are represented by formula (B3a-1), formula (B3a-2), and formula (B3a-12) to formula (B3a-14).

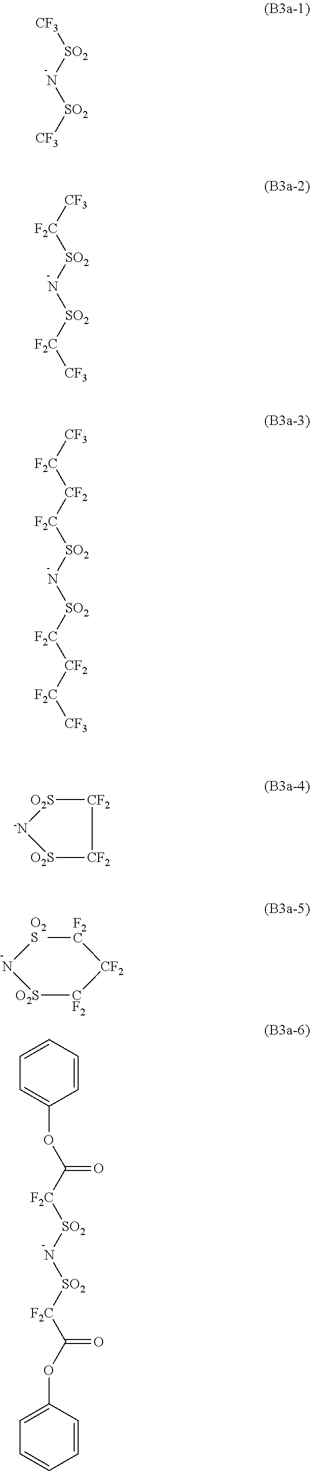

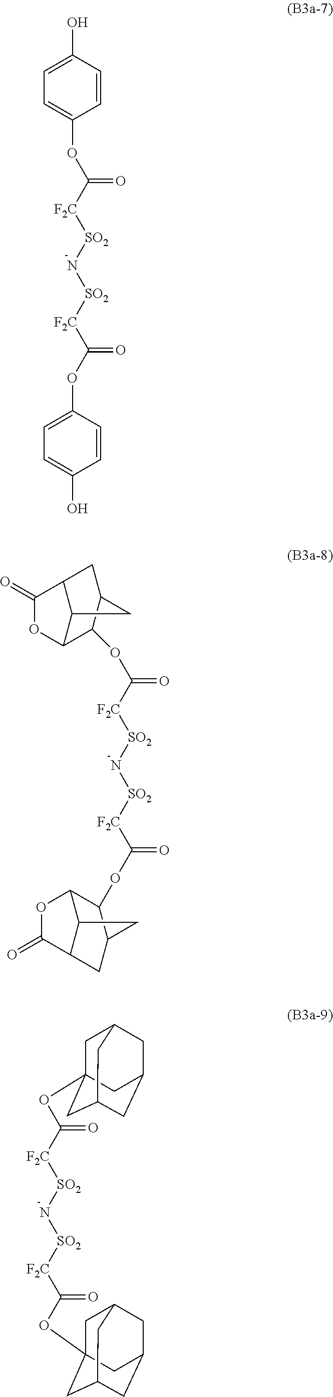 [SMILES: O=C(Oc1ccc(O)cc1)C(F)(F)S(=O)(=O)NS(=O)(=O)C(F)(F)C(=O)Oc1ccc(O)cc1 ; O=C1OC2C3CC(CC13)C2OC(=O)C(F)(F)S(=O)(=O)NS(=O)(=O)C(F)(F)C(=O)OC1C2CC3C(=O)OC1C3C2 ; O=C(OC12CC3CC(CC(C3)C1)C2)C(F)(F)S(=O)(=O)NS(=O)(=O)C(F)(F)C(=O)OC12CC3CC(CC(C3)C1)C2]

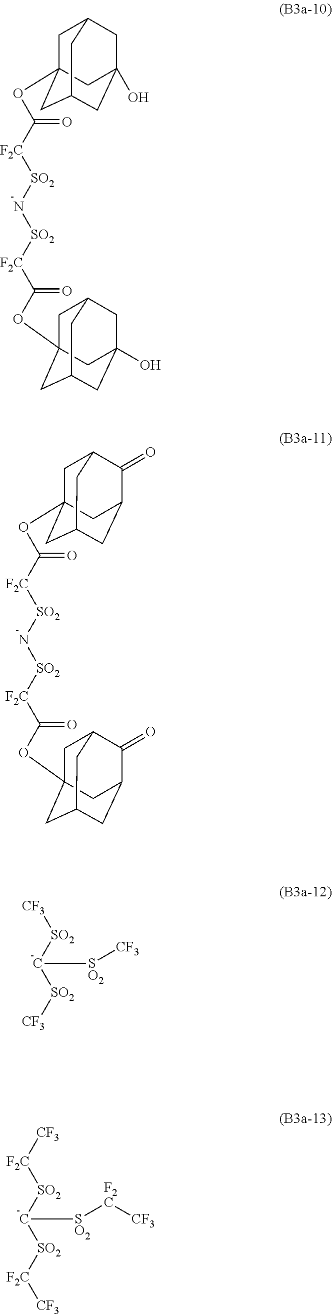 [SMILES: O=C(OC12CC3CC(CC(O)(C3)C1)C2)C(F)(F)S(=O)(=O)[N-]S(=O)(=O)C(F)(F)C(=O)OC12CC3CC(CC(O)(C3)C1)C2 ; O=C1C2CC3CC1CC(OC(=O)C(F)(F)S(=O)(=O)[N-]S(=O)(=O)C(F)(F)C(=O)OC14CC5CC(C1)C(=O)C(C5)C4)(C3)C2 ; O=S(=O)([C-](S(=O)(=O)C(F)(F)F)S(=O)(=O)C(F)(F)F)C(F)(F)F ; O=S(=O)(C(S(=O)(=O)C(F)(F)C(F)(F)F)S(=O)(=O)C(F)(F)C(F)(F)F)C(F)(F)C(F)(F)F]

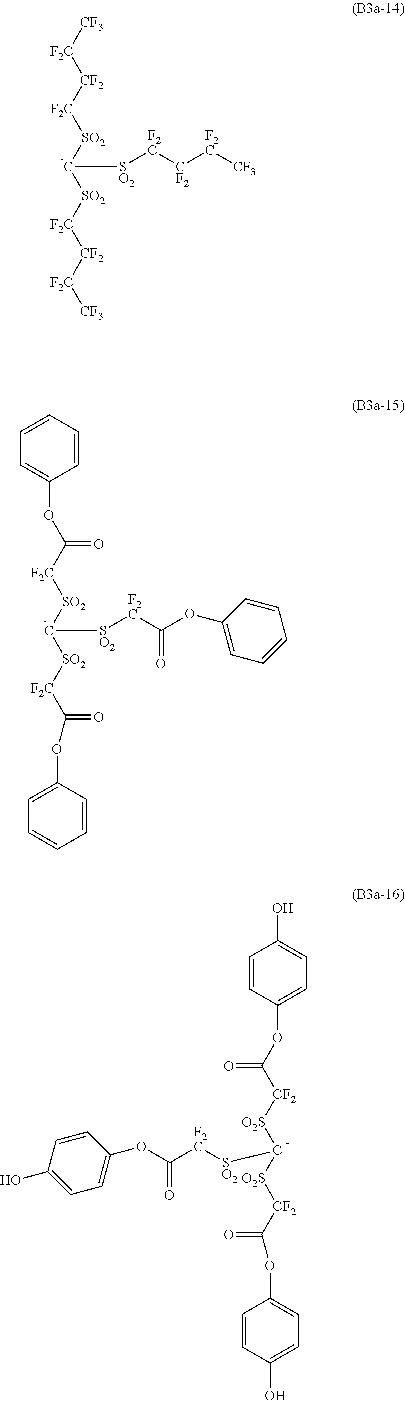 [SMILES: O=S(=O)(C(S(=O)(=O)C(F)(F)C(F)(F)C(F)(F)C(F)(F)F)S(=O)(=O)C(F)(F)C(F)(F)C(F)(F)C(F)(F)F)C(F)(F)C(F)(F)C(F)(F)C(F)(F)F ; O=C(Oc1ccccc1)C(F)(F)S(=O)(=O)C(S(=O)(=O)C(F)(F)C(=O)Oc1ccccc1)S(=O)(=O)C(F)(F)C(=O)Oc1ccccc1 ; O=C(Oc1ccc(O)cc1)C(F)(F)S(=O)(=O)C(S(=O)(=O)C(F)(F)C(=O)Oc1ccc(O)cc1)S(=O)(=O)C(F)(F)C(=O)Oc1ccc(O)cc1]

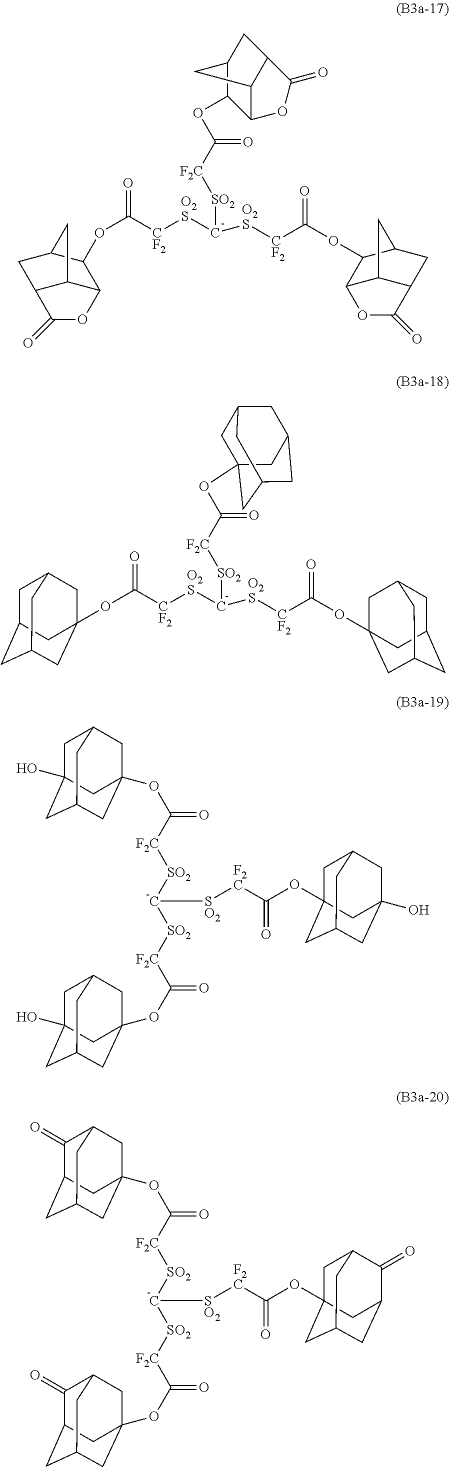

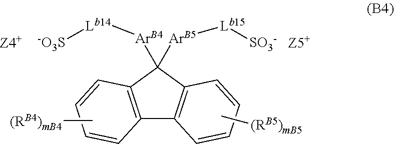

wherein in formula (B4),
L^{b14} and L^{b15} each independently represent a single bond, or a divalent hydrocarbon group having 1 to 72 carbon atoms and optionally having a substituent, and —CH₂— contained in the hydrocarbon group is optionally replaced with —O—, —S—, —CO—, —SO—, or —SO₂—;
Ar^{B4} and Ar^{B5} each independently represent an aromatic hydrocarbon group having 6 to 24 carbon atoms and optionally having a substituent, and the substituents of Ar^{B4} and Ar^{B5} may be combined to form a ring;
R^{B4} and R^{B5} each independently represent a halogen atom or a hydrocarbon group having 1 to 18 carbon atoms and optionally having a substituent, and —CH₂— contained in the hydrocarbon group is optionally replaced with —O—, —CO—, —S—, —SO—, or —SO₂—;
mB4 represents an integer of 0 to 4, and when mB4 is 2 or larger, a plurality of R^{B4} may be the same or different from each other;
mB5 represents an integer of 0 to 4, and when mB5 is 2 or larger, a plurality of R^{B5} may be the same or different from each other;
Z4⁺ and Z5⁺ each independently represent an organic cation.

In formula (B4), examples of L^{b14} and L^{b15} include the same groups as L^{b1} in formula (B1) or L^{b3} in formula (B1-A1).

Examples of the aromatic hydrocarbon groups for Ar^{B4} and Ar^{B5} include a phenylene group, a naphthylene group, an anthrylene group, a biphenylene group and a phenanthrylene group. The bonding site can be set to any position. The number of carbon atoms of the aromatic hydrocarbon group is preferably 6 to 18, is more preferably 6 to 14, and is further preferably 6 to 10.

Examples of the substituent that may be included in the aromatic hydrocarbon groups of Ar^{B4} and Ar^{B5} include a halogen atom, a hydroxyl group, a cyano group, a carboxyl group, an alkyl group having 1 to 12 carbon atoms, a fluorinated alkyl group having 1 to 6 carbon atoms, an alkoxy group having 1 to 12 carbon atoms, and groups formed by combining two or more of these groups. Examples of these substituents include the same as those exemplified in R^{B4} and R^{B5} that will be described later.

Among them, Ar^{B4} and Ar^{B5} are preferably an aromatic hydrocarbon group having 6 to 10 carbon atoms and optionally having a substituent, more preferably an aromatic hydrocarbon group having 6 to 10 carbon atoms and optionally having a halogen atom, a hydroxyl group, an alkyl group having 1 to 12 carbon atoms, or a fluorinated alkyl group having 1 to 6 carbon atoms, still more preferably an aromatic hydrocarbon group having 6 to 10 carbon atoms and optionally having a fluorine atom, an iodine atom, an alkyl group having 1 to 6 carbon atoms, or a fluorinated alkyl group having 1 to 3 carbon atoms, and even still more preferably a phenylene group or a naphthalene group.

When Ar^{B4} and Ar^{B5} are phenylene groups that may have substituents, the bonding position of the fluorene ring may be the ortho position, the meta position, or the para position with respect to the bonding positions of L^{b24} and L^{b25}, but is preferably the meta position or the para position, and more preferably the para position.

Examples of the halogen atom for R^{B4} and R^{B5} include a fluorine atom, a chlorine atom, a bromine atom and an iodine atom.

Examples of the hydrocarbon groups having 1 to 18 carbon atoms for R^{B4} and R^{B5} include chain hydrocarbon groups such as an alkyl group and an alkanediyl group, alicyclic hydrocarbon groups, aromatic hydrocarbon groups, and groups formed by combining these groups.

The alkyl group is a straight-chain or branched alkyl group, and examples thereof include a methyl group, an ethyl group, a n-propyl group, an isopropyl group, a n-butyl group, a sec-butyl group, a tert-butyl group, a pentyl group, a hexyl group, a heptyl group, a 2-ethylhexyl group, an octyl group, a nonyl group, a decyl group, an undecyl group, and a dodecyl group.

The alkanediyl group is a straight-chain or branched alkanediyl group, and examples thereof include straight-chain alkanediyl groups such as a methylene group, an ethylene group, a propane-1,3-diyl group, a butane-1,4-diyl group, a pentane-1,5-diyl group, and a hexane-1,6-diyl group; and
branched alkanediyl groups such as an ethane-1,1-diyl group, a propane-1,1-diyl group, a propane-1,2-diyl group, a propane-2,2-diyl group, a pentane-2,4-diyl group, a 2-methylpropane-1,3-diyl group, a 2-methylpropane-1,2-diyl group, a pentane-1,4-diyl group and a 2-methylbutane-1,4-diyl group.

The number of carbon atoms of the chain hydrocarbon group is preferably 1 to 12, is more preferably 1 to 9, is further preferably 1 to 6, is far more preferably 1 to 4, and is still further preferably 1 to 3.

The alicyclic hydrocarbon group may be either monocyclic or polycyclic, and examples thereof include the groups represented below. The bonding site can be set to any position.

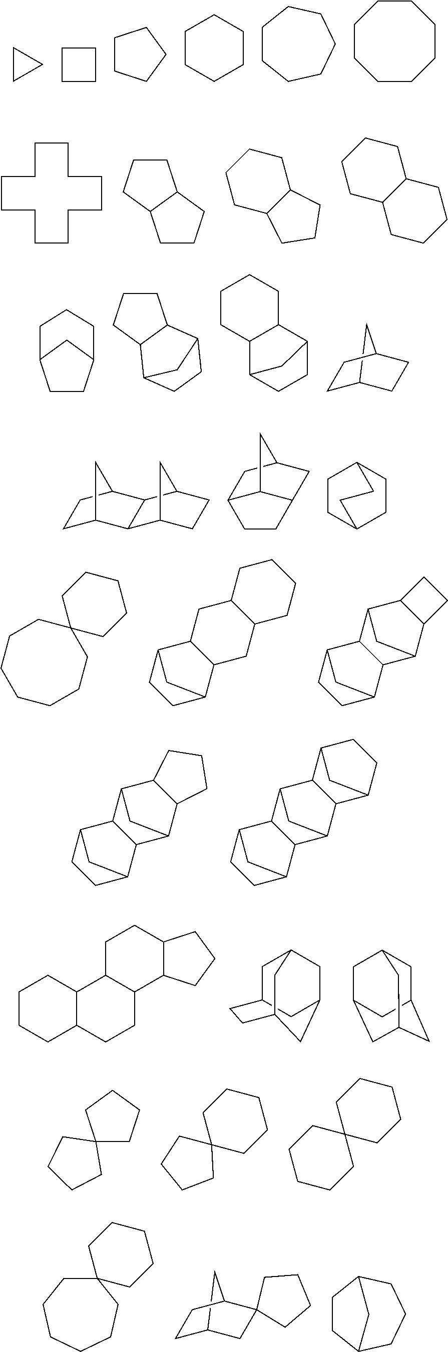

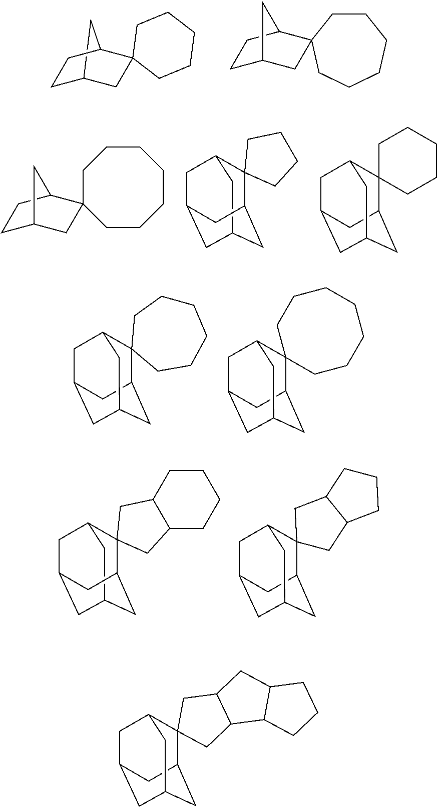

Specific examples of the monocyclic alicyclic hydrocarbon group include monocyclic cycloalkyl groups such as a cyclopropyl group, a cyclobutyl group, a cyclopentyl group, a cyclohexyl group, a cycloheptyl group, a cyclooctyl group, a cyclononyl group, a cyclodecyl group, and a cyclododecyl group. Examples of the polycyclic alicyclic hydrocarbon group include: polycyclic cycloalkyl groups such as a decahydronaphthyl group, an adamantyl group, and a norbornyl group; or spiro rings having a cycloalkyl group bonded to each of a cycloalkyl group, a norbornyl group and an adamantyl group by spiro, such as a spirocyclohexane-1,2′ cyclopentane group and a spiroadamantane-2,3′-cyclopentane group. The number of carbon atoms of the alicyclic hydrocarbon group is preferably 3 to 18, is more preferably 3 to 16, and is further preferably 3 to 12.

Examples of the aromatic hydrocarbon group include a phenyl group, a naphthyl group, a biphenyl group, an anthryl group, a phenanthryl group and a binaphthyl group. The number of carbon atoms of the aromatic hydrocarbon group is preferably 6 to 18, is more preferably 6 to 14, and is further preferably 6 to 10.

Examples of the groups formed by combining two or more groups of the chain hydrocarbon group, the alicyclic hydrocarbon group, and the aromatic hydrocarbon group include a combined group of an aromatic hydrocarbon group and a chain hydrocarbon group (for example, aromatic hydrocarbon group- alkanediyl group-*, alkyl group-aromatic hydrocarbon group-*, alkyl group-aromatic hydrocarbon group-alkanediyl group-*, and —CH₂— contained in the alkanediyl group and the alkyl group is optionally replaced with —O—, —CO—, —S—, —SO—, or —SO₂—.), a combined group of an alicyclic hydrocarbon group and a chain hydrocarbon group (for example, alicyclic hydrocarbon group- alkanediyl group-*, alkyl group-alicyclic hydrocarbon group-*, alkyl group-alicyclic hydrocarbon group- alkanediyl group-*, and —CH₂— contained in the alicyclic hydrocarbon group, the alkanediyl group and the alkyl group is optionally replaced with —O—, —CO—, —S—, —SO—, or —SO₂—.), and a combined group of an aromatic hydrocarbon group and an alicyclic hydrocarbon group (for example, aromatic hydrocarbon group-alicyclic hydrocarbon group-*, alicyclic hydrocarbon group-aromatic hydrocarbon group-*, —CH₂— contained in the alicyclic hydrocarbon group is optionally replaced with —O—, —CO—, —S—, —SO—, or —SO₂—.). * represents a bonding site.

Examples of the aromatic hydrocarbon group- alkanediyl group-* include aralkyl groups such as a benzyl group and a phenethyl group.

Examples of the alkyl group-aromatic hydrocarbon group-* include a tolyl group, a xylyl group, and a cumenyl group.

Examples of the alicyclic hydrocarbon group- alkanediyl group-* include a cyclohexylmethyl group, a cyclohexylethyl group, a 1-(adamantan-1-yl)methyl group, and a 1-(adamantan-1-yl)-1-methylethyl group.

Examples of the alkyl group-alicyclic hydrocarbon group-* include cycloalkyl groups having an alkyl group such as a methylcyclohexyl group, a dimethylcyclohexyl group, and a 2-alkyladamantan-2-yl group.

Examples of the aromatic hydrocarbon group-alicyclic hydrocarbon group-* include a phenylcyclohexyl group.

Examples of the alicyclic hydrocarbon group-aromatic hydrocarbon group-* include a cyclohexylphenyl group.

Here, in the combination, two or more types of alicyclic hydrocarbon groups, aromatic hydrocarbon groups, and chain hydrocarbon groups may each be combined. In addition, any of the groups may be bonded to a benzene ring or the like.

When —CH₂— contained in the hydrocarbon groups represented by R^{B4} and R^{B5} is replaced with —O—, —CO—, —S—, —SO—, or —SO₂—, the number of carbon atoms before replacement corresponds to the defined number of carbon atoms in the hydrocarbon group. Furthermore, its number may be one, or may be two or more.

Examples of the group formed by replacing —CH₂— contained in the hydrocarbon group with —O—, —S—, —SO₂—, —SO—, or —CO— include: as described above, the hydroxyl group, the thiol group, the carboxy group, the alkoxy group, the alkoxycarbonyl group, the alkylcarbonyl group, the alkylcarbonyloxy group, the alkylthio group, the alkylsulfonyl group, the oxy group, the carbonyl group, the thio group, the sulfonyl group, the alkanediyloxy group, the alkanediyloxycarbonyl group, the alkanediylcarbonyl group, the alkanediylcarbonyloxy group, the alkanediylthio group, the alkanediylsulfonyl group, the cycloalkoxy group, the cycloalkylalkoxy group, the alkoxycarbonyloxy group, an aromatic hydrocarbon group-carbonyloxy group, an aromatic hydrocarbon group-carbonyl group, an aromatic hydrocarbon group-oxy group, and a combined group of two or more of these groups. Examples of these replaced groups include the same groups as those exemplified in the present specification, within the acceptable range of the upper limit of the number of carbon atoms.

In addition, examples of the group formed by replacing —CH₂— contained in the alicyclic hydrocarbon group, and the combined group of the alicyclic hydrocarbon group and the aromatic hydrocarbon group with —O—, —S—, —SO₂—, —SO—, or —CO— include the same groups as those exemplified in the present specification, within the acceptable range of the upper limit of the number of carbon atoms.

Examples of substituents that the hydrocarbon groups for R^{B4} and R^{B5} optionally have include a halogen atom, a cyano group, and a nitro group.

Examples of the halogen atom include the same groups as those described above.

The hydrocarbon group optionally has one substituent or a plurality of substituents.

When R^{B4} and R^{B5} are hydrocarbon groups having a halogen atom, examples of the hydrocarbon groups having a halogen atom for R^{B4} and R^{B5} include chain hydrocarbon groups having a halogen atom such as haloalkyl groups, alicyclic hydrocarbon groups having a halogen atom, and aromatic hydrocarbon groups having a halogen atom.

The haloalkyl group refers to an alkyl group having a halogen atom, and examples thereof include haloalkyl groups having 1 to 12 carbon atoms, such as fluorinated alkyl groups having 1 to 12 carbon atoms, chlorinated alkyl groups having 1 to 12 carbon atoms, brominated alkyl groups having 1 to 12 carbon atoms, and iodinated alkyl groups having 1 to 12 carbon atoms. Examples of the haloalkyl group include a perfluoroalkyl group having 1 to 12 carbon atoms (such as a trifluoromethyl group, a pentafluoroethyl group, a heptafluoropropyl group, and a nonafluorobutyl group), a 2,2,2-trifluoroethyl group, a 3,3,3-trifluoropropyl group, a 4,4,4-trifluorobutyl group, a 3,3,4,4,4-pentafluorobutyl group, a chloromethyl group, a bromomethyl group, and an iodomethyl group. The number of carbon atoms in the haloalkyl group is preferably 1 to 9, more preferably 1 to 6, further preferably 1 to 4, and still more preferably 1 to 3.

The —CH₂— contained in the haloalkyl group is optionally replaced with —O— or —CO— or the like, and examples of such groups include haloalkoxy groups, haloalkoxycarbonyl groups, haloalkylcarbonyl groups, and haloalkylcarbonyloxy groups. Specifically, examples thereof include haloalkoxy groups having 1 to 11 carbon atoms, haloalkoxycarbonyl groups having 2 to 11 carbon atoms, haloalkylcarbonyl groups having 2 to 12 carbon atoms, and haloalkylcarbonyloxy groups having 2 to 11 carbon atoms, and specific examples thereof include groups obtained by replacing one or more hydrogen atoms of the groups exemplified above with a halogen atom.

mB4 is preferably 0, 1, 2, or 3, more preferably 0, 1, or 2, and further preferably 0 or 1.

mB5 is preferably 0, 1, 2, or 3, more preferably 0, 1, or 2, and further preferably 0 or 1.

R^{B4} and R^{B5} are each independently preferably a halogen atom, a haloalkyl group having 1 to 6 carbon atoms, or an alkyl group having 1 to 6 carbon atoms (—CH₂— contained in the haloalkyl group and the alkyl group is optionally replaced with —O— or —CO—.), more preferably a fluorine atom, an iodine atom, a haloalkyl group having 1 to 6 carbon atoms, or an alkyl group having 1 to 6 carbon atoms (—CH₂— contained in the haloalkyl group and the alkyl group is optionally replaced with —O— or —CO—.), further preferably a fluorine atom, an iodine atom, a haloalkyl group having 1 to 4 carbon atoms, or an alkyl group having 1 to 4 carbon atoms (—CH₂— contained in the alkyl group is optionally replaced with —O— or —CO—.), still more preferably a fluorine atom, an iodine atom, a hydroxy group, an alkyl group having 1 to 4 carbon atoms, a haloalkyl group having 1 to 4 carbon atoms, or an alkoxy group having 1 to 3 carbon atoms, even still more preferably a fluorine atom, an iodine atom, a hydroxy group, an alkyl group having 1 to 4 carbon atoms, a perfluoroalkyl group having 1 to 4 carbon atoms, or an alkoxy group having 1 to 3 carbon atoms, and even further more preferably a fluorine atom, an iodine atom, a trifluoromethyl group, a methyl group, a methoxy group, an ethoxy group, or a hydroxy group.

Examples of the anion in the salt represented by formula (B4) include the following. Among these, anions are preferable which are represented by formula (B4a-1) to formula (B4a-4), and formula (B4a-11) to formula (B4a-14).

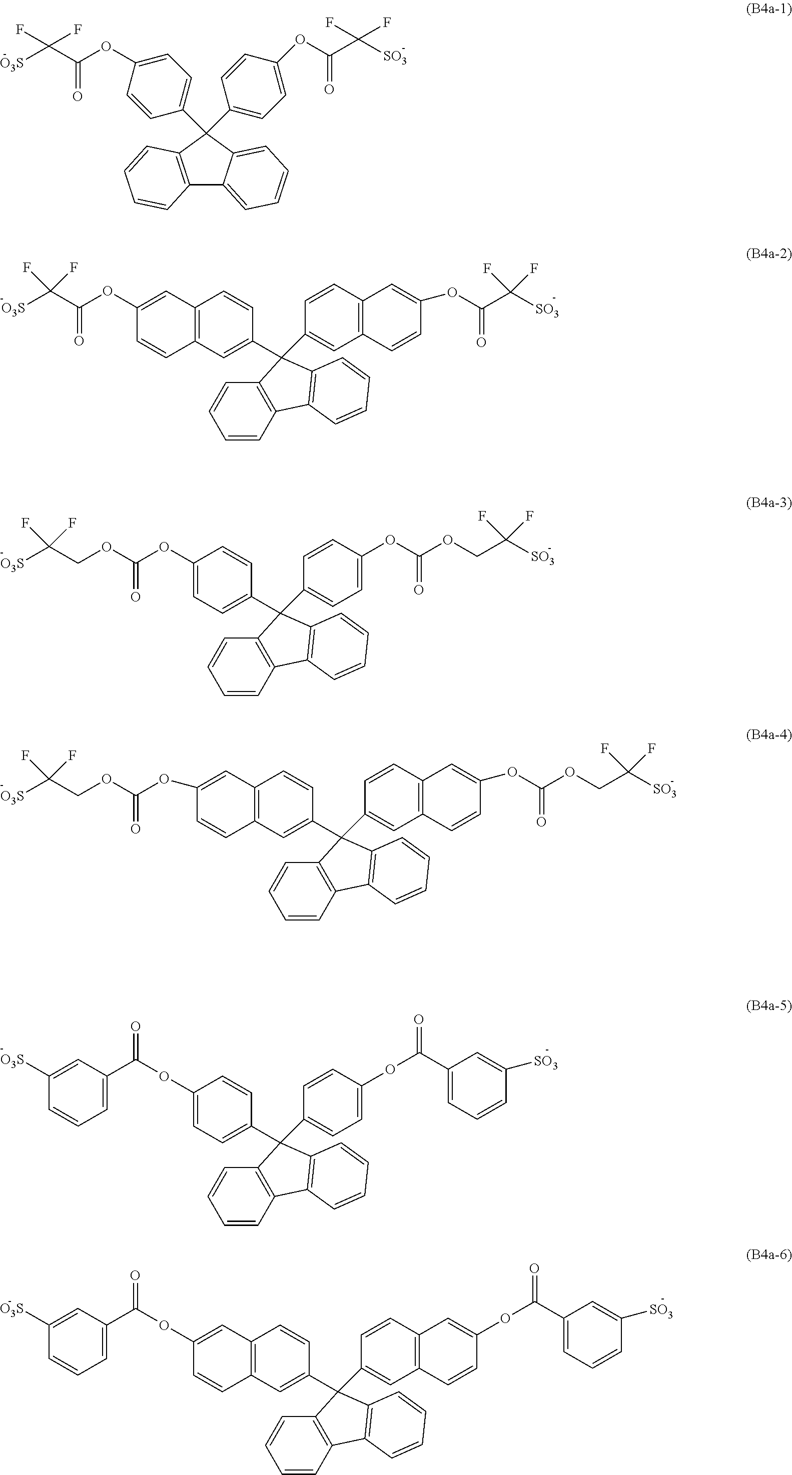

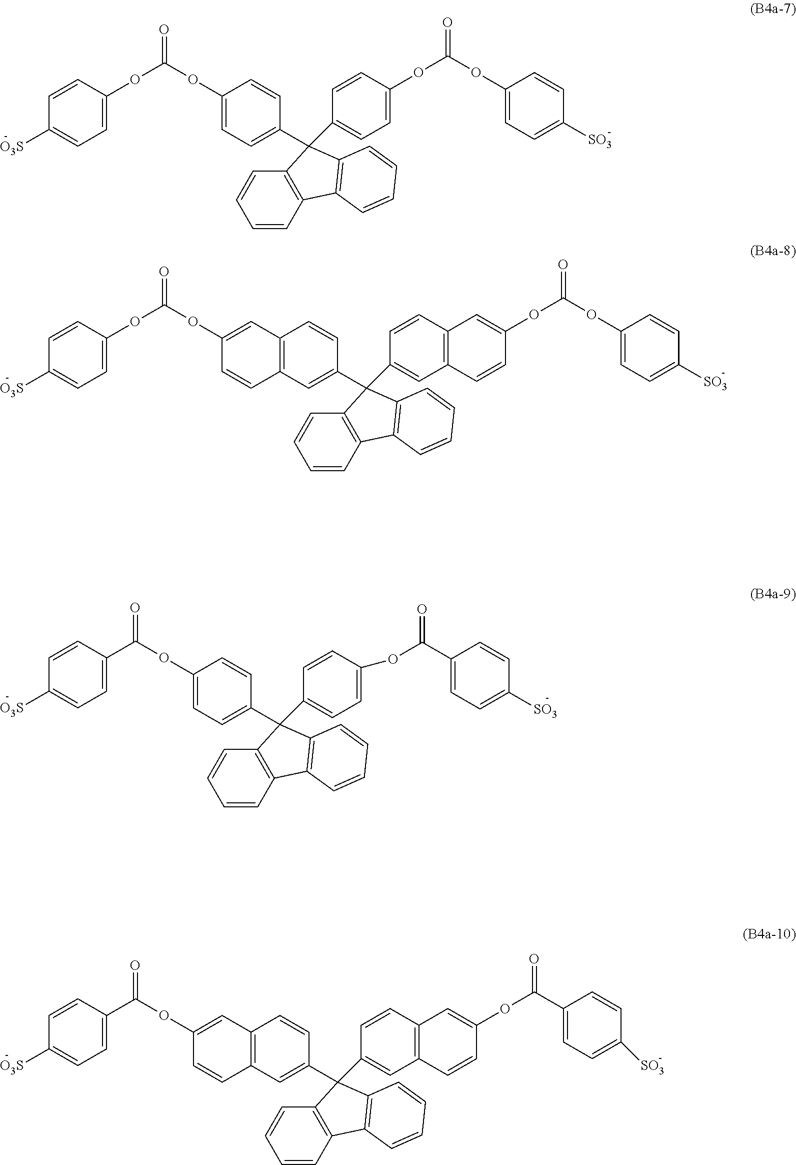

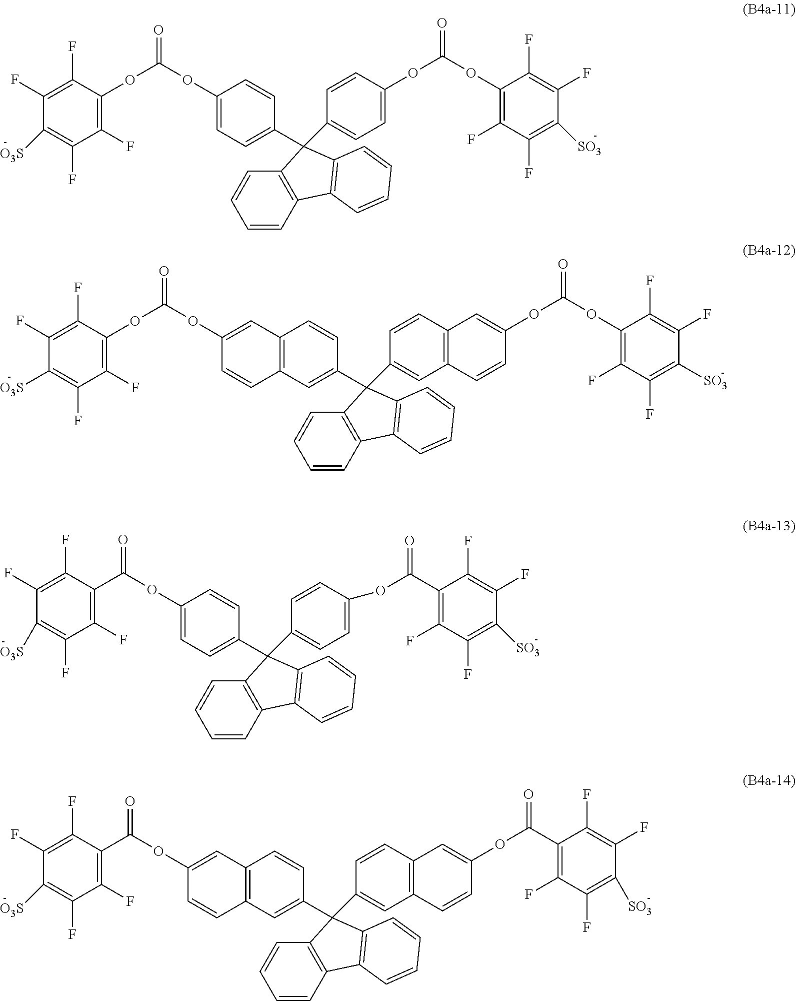

In addition, in another embodiment of the compound (B), a salt obtained by replacing the anion in the salt represented by formula (B 1) with a carboxylate anion is also suitably used as the compound (B).

Examples of the carboxylate anion include the following anions.

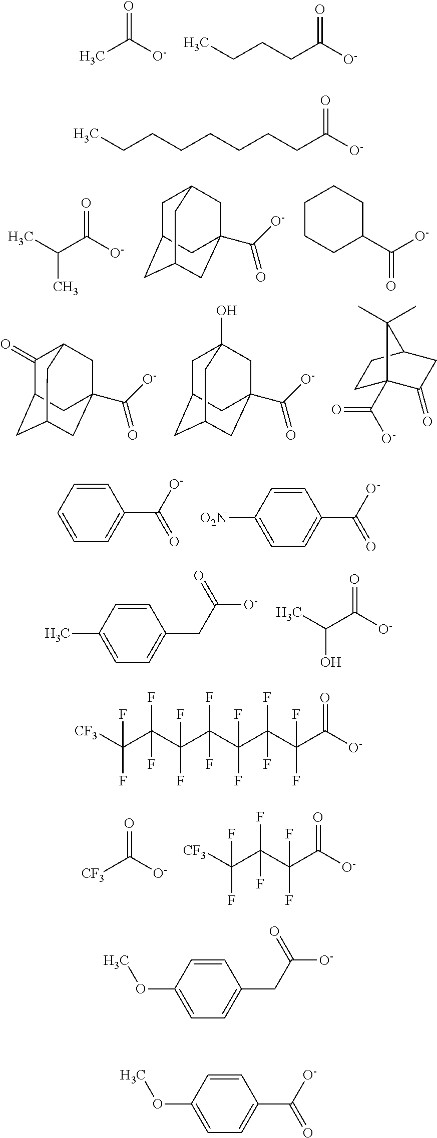

Examples of the organic cation of Z1⁺, Z2⁺, Z4⁺, and Z5⁺ include the same cations as the organic cations of Z⁺ in formula (I).

The compound (B) is a combination of the above-described anions and the above-described organic cations, and these anions and cations can be arbitrarily combined. Preferable examples of the compound (B) include combinations of an anion represented by any of formula (B1a-1) to formula (B1a-4), formula (B1a-7) to formula (B1a-11), formula (B1a-14) to formula (B1a-30), formula (B1a-35) to formula (B1a-70), formula (B2a-1) to formula (B2a-11), formula (B2a-16) to formula (B2a-20), formula (B3a-1) to formula (B3a-3), formula (B3a-11) to formula (B3a-14), formula (B4a-1) to formula (B4a-4), or formula (B4a-11) to formula (B4a-14) with a cation (b2-1), a cation (b2-2), a cation (b2-3), a cation (b2-4), or a cation (b2-5).

Preferable examples of the compound (B) include compounds represented by each of formula (B1-1) to formula (B1-106), formula (B2-1) to formula (B2-20), formula (B3-1) to formula (B3-28), and formula (B4-1) to formula (B4-16). Among the compounds, compounds are preferable which contain an arylsulfonium cation, and compounds are particularly preferable which are represented by formula (B1-1) to formula (B1-3), formula (B1-5) to formula (B1-7), formula (B1-11) to formula (B1-14), formula (B1-20) to formula (B1-26), formula (B1-29), formula (B1-31) to formula (B1-106), formula (B2-1) to formula (B2-20), formula (B3-1) to formula (B3-28), and formula (B4-1) to formula (B4-16).

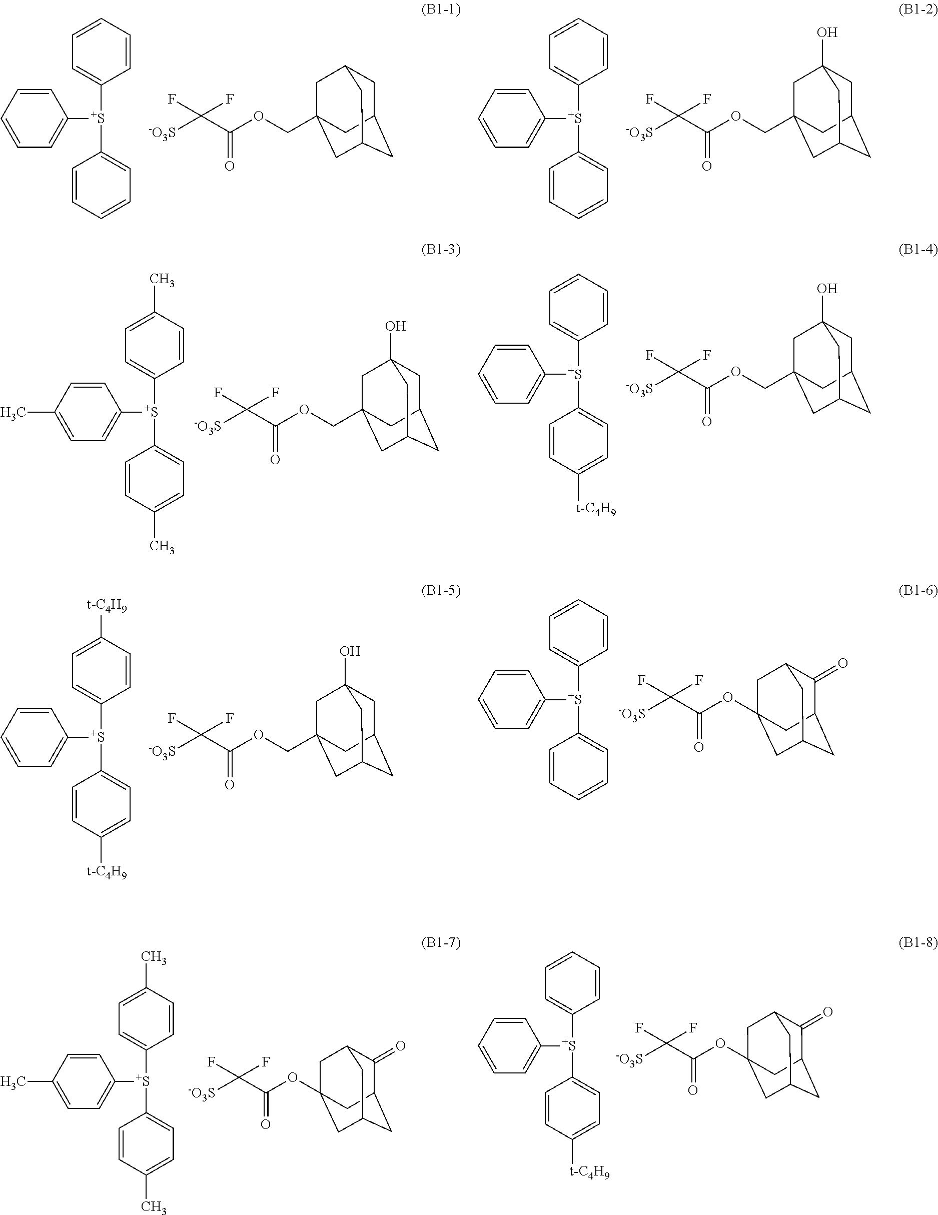

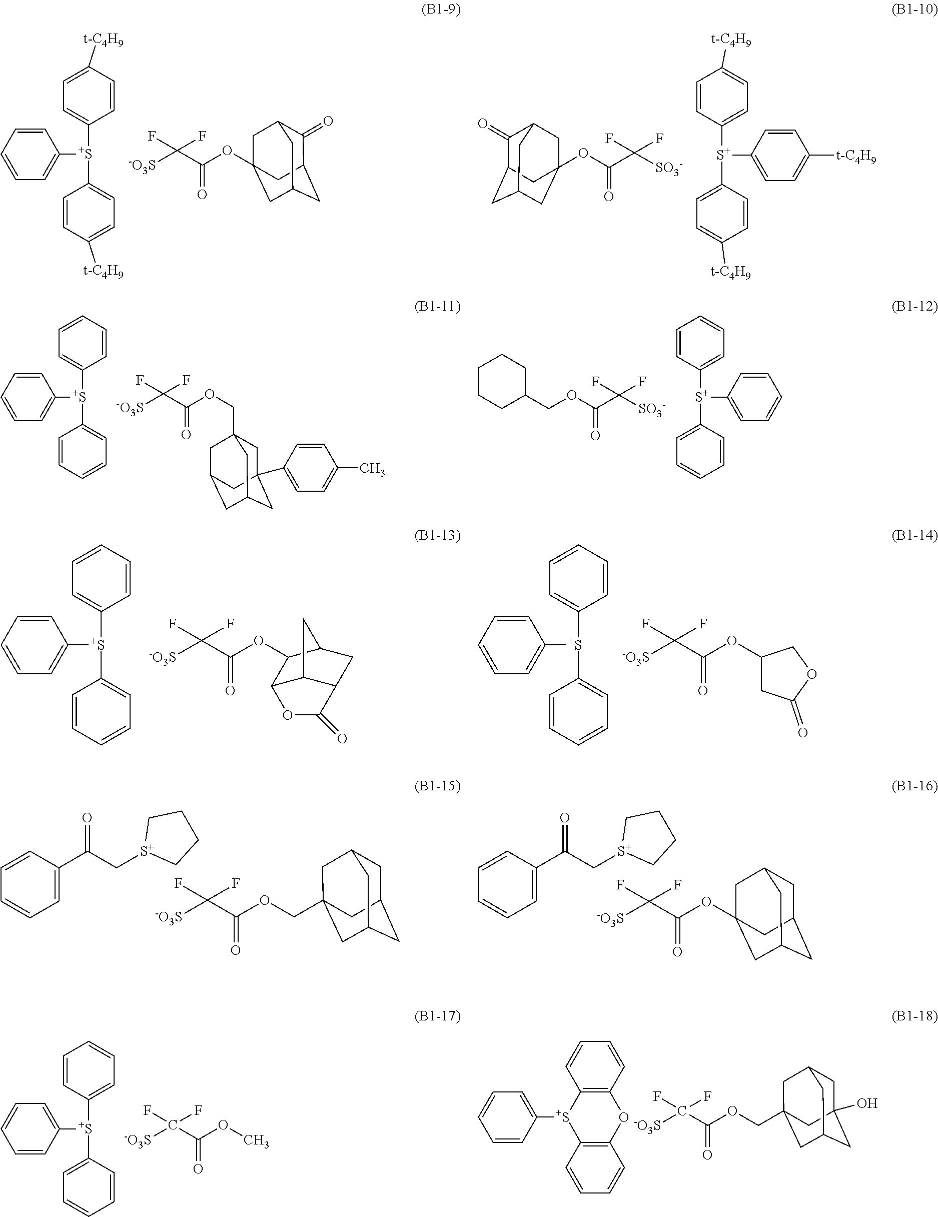

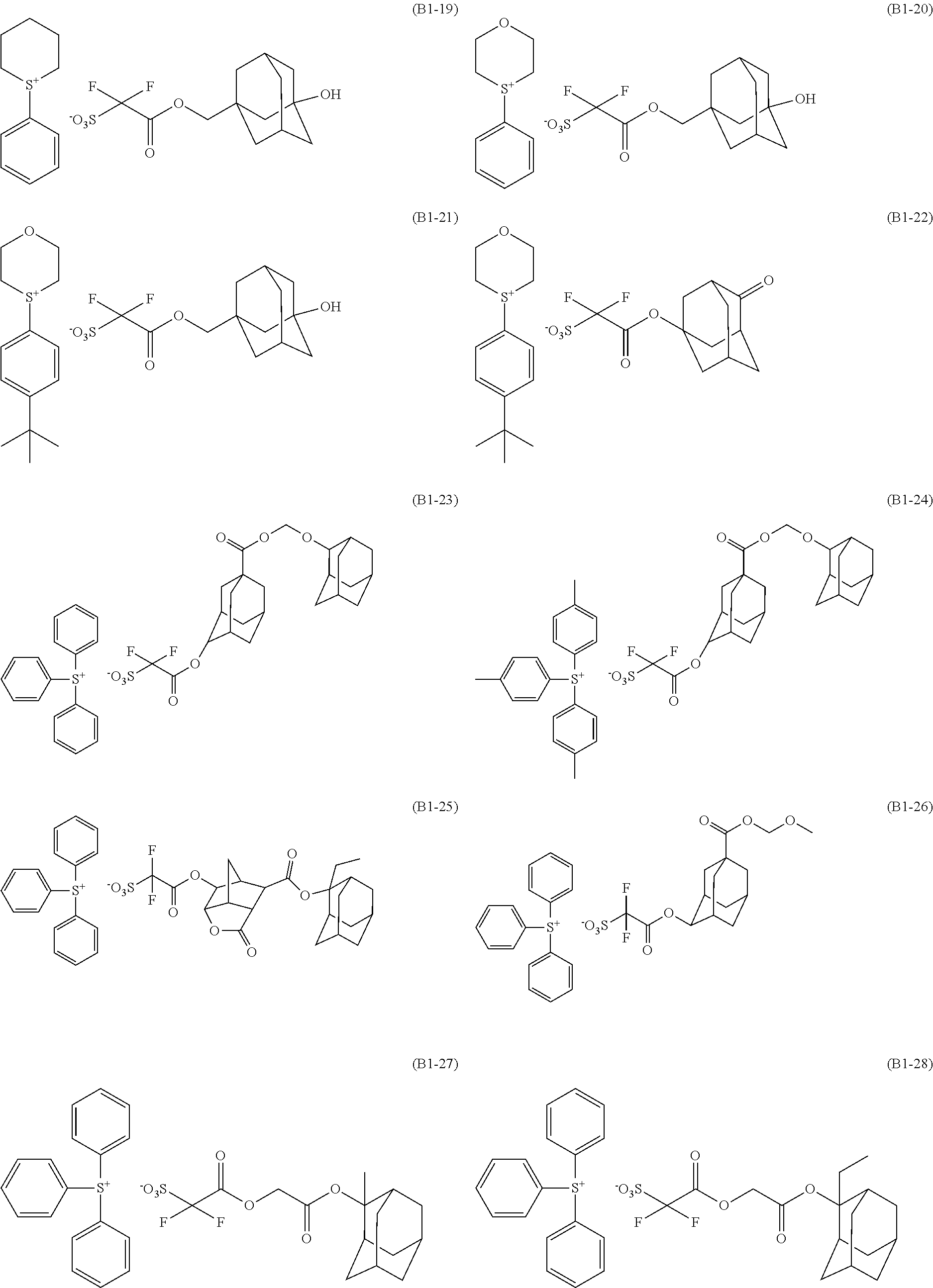

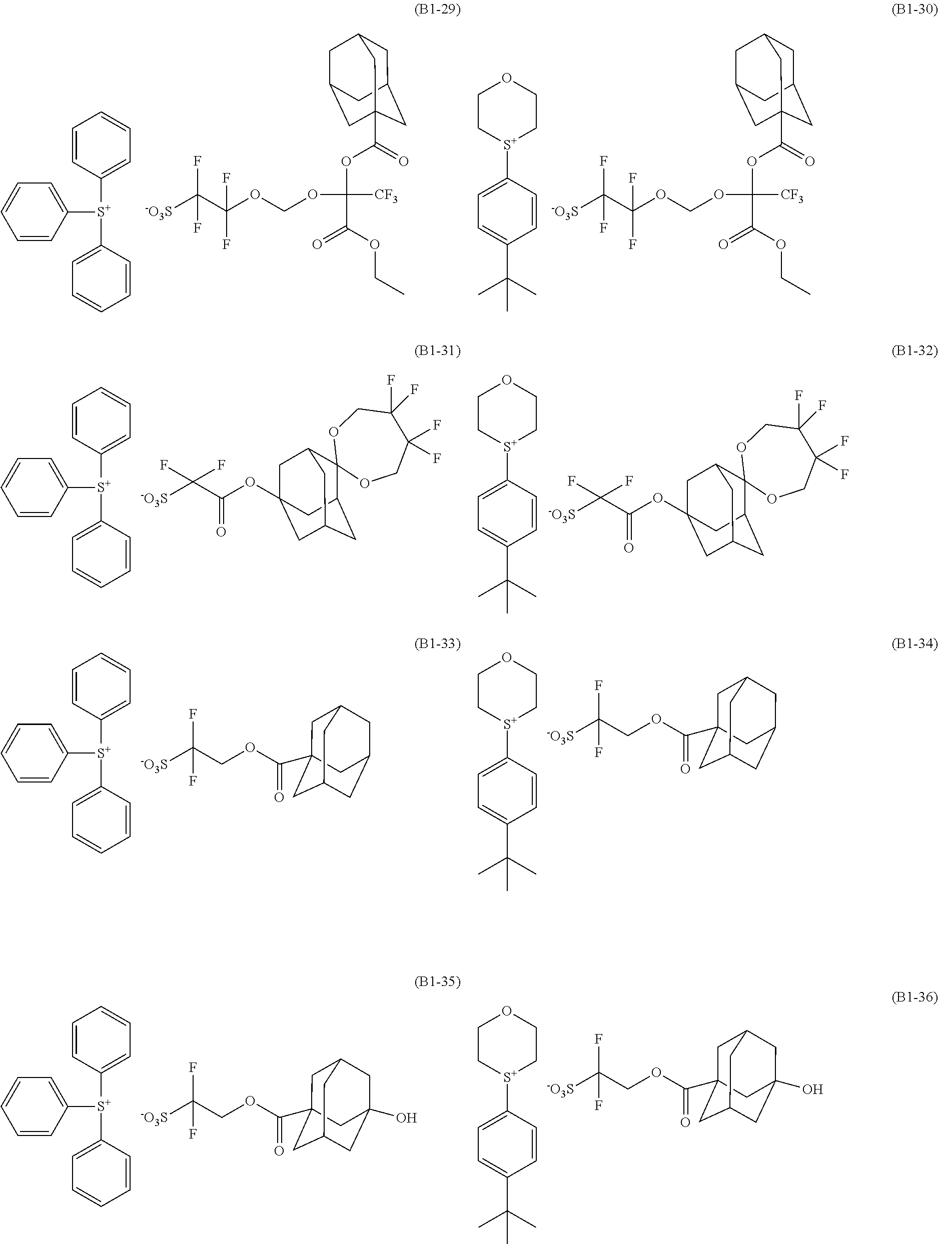

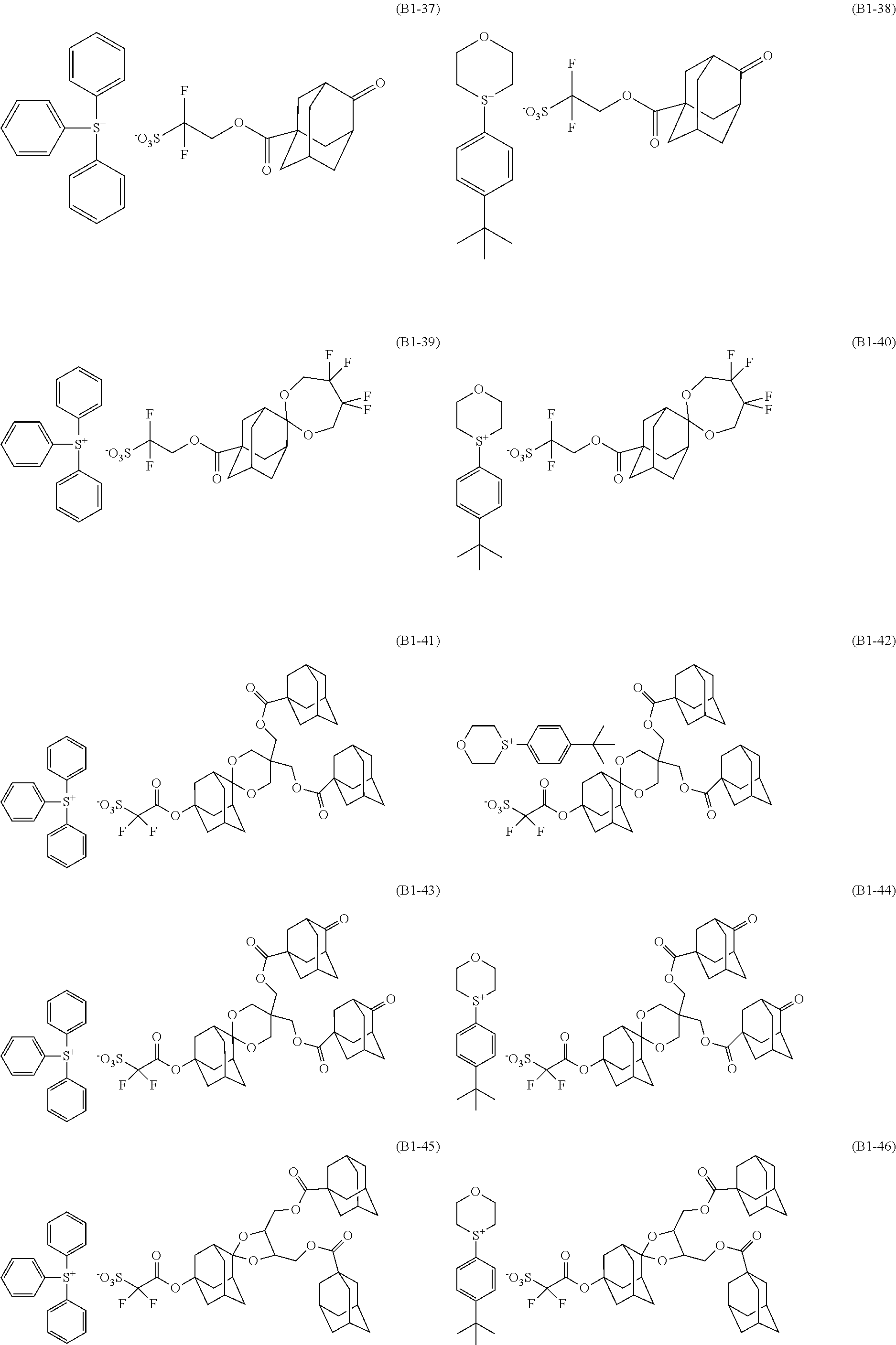

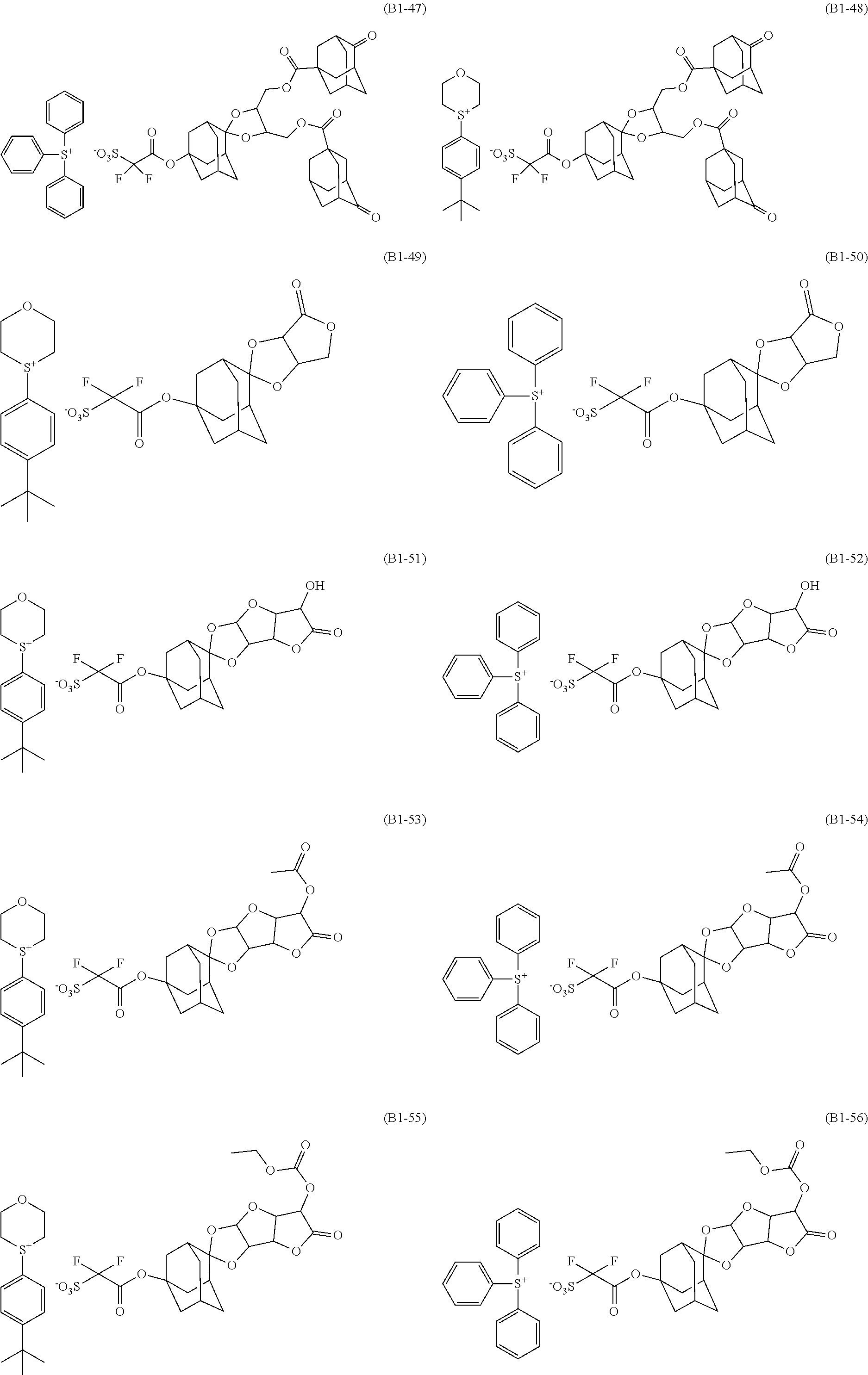

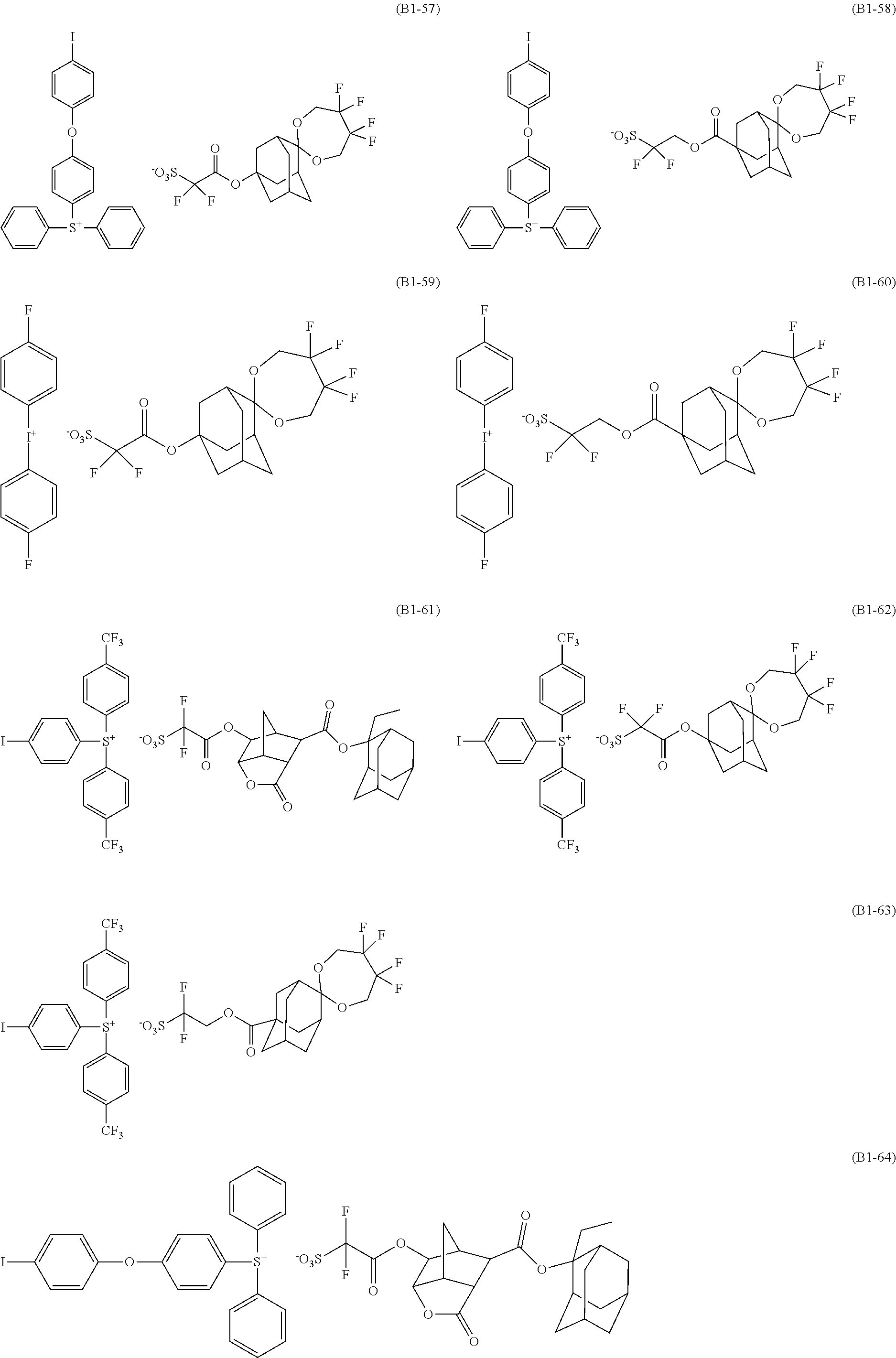

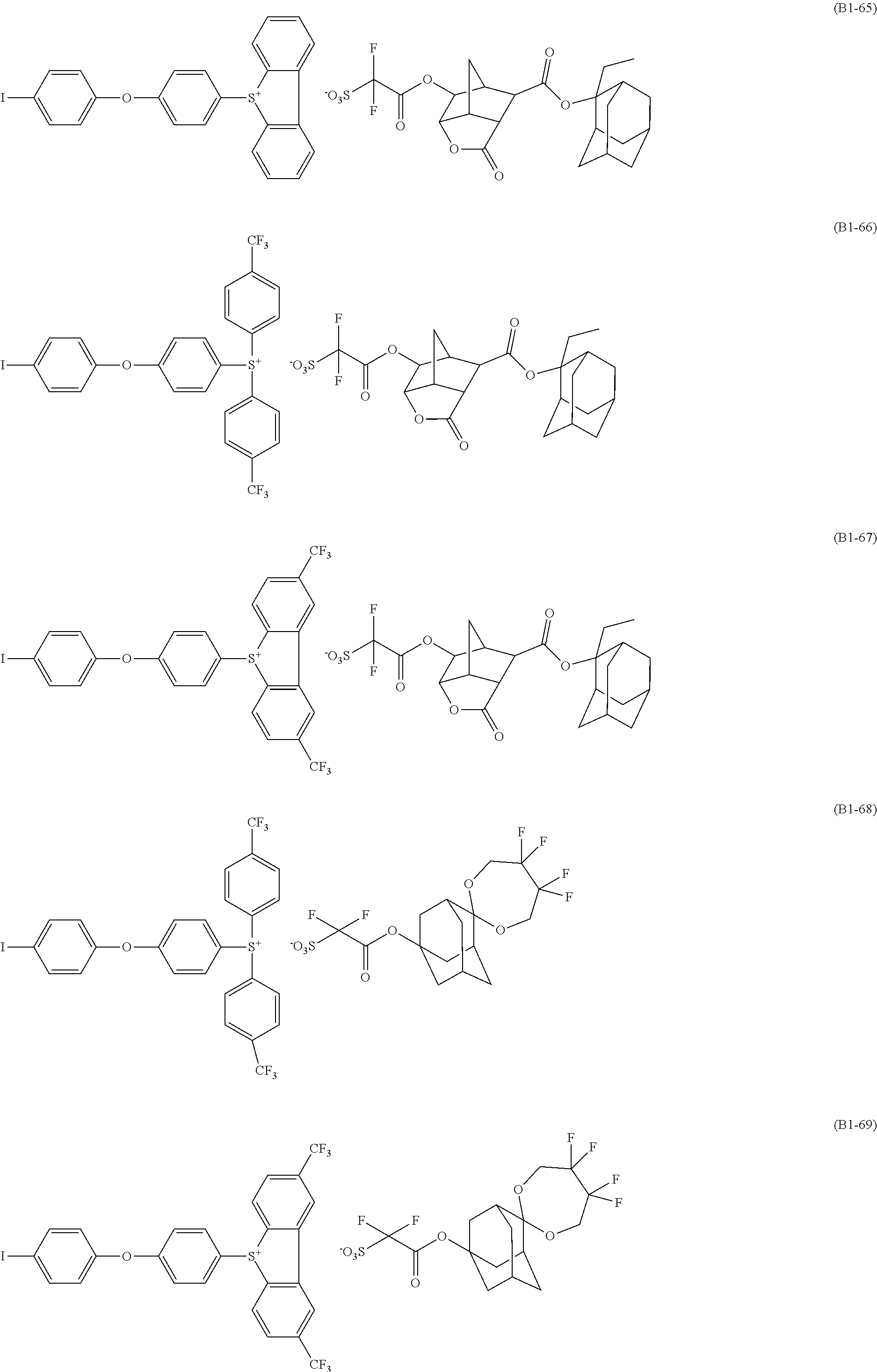

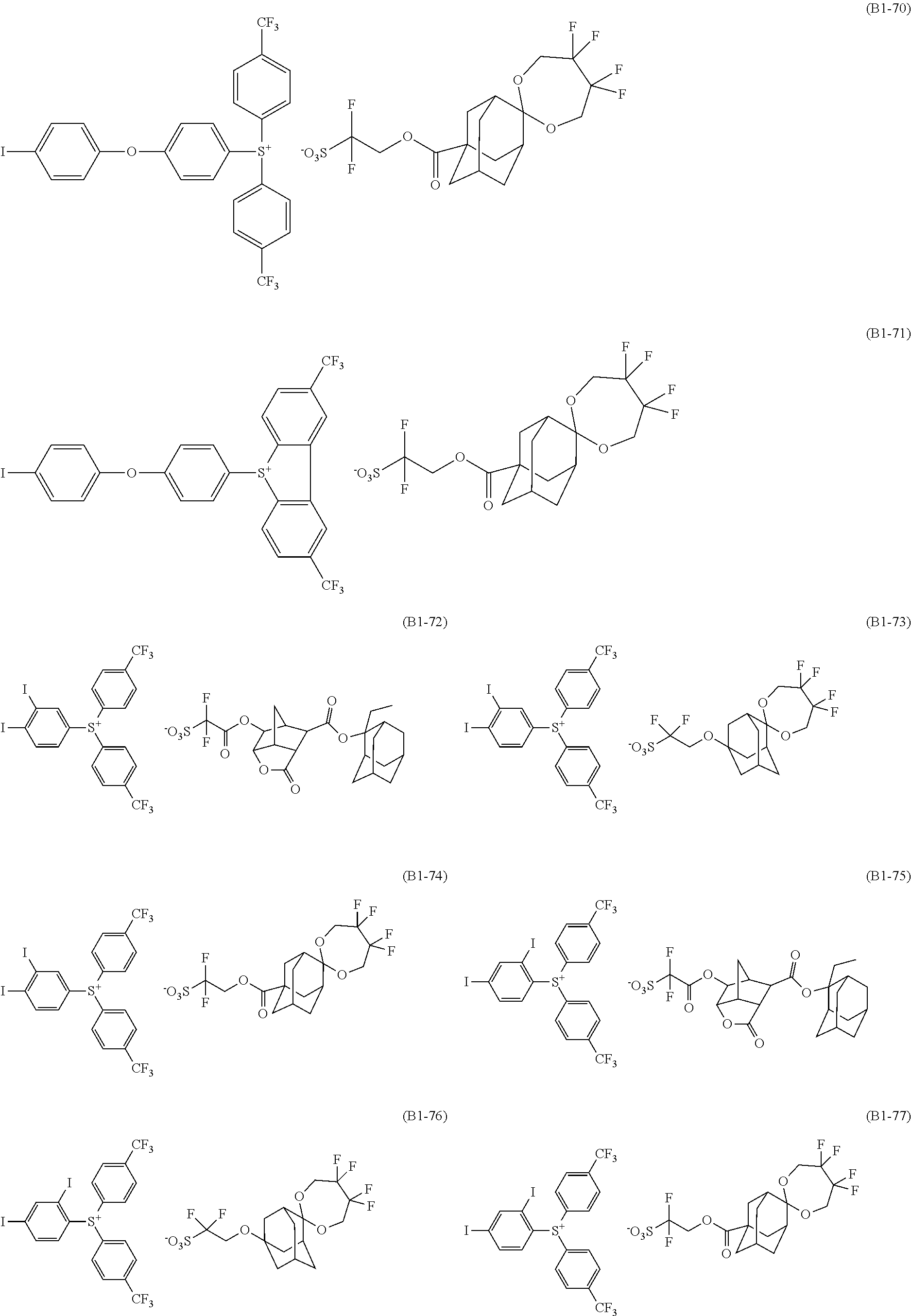

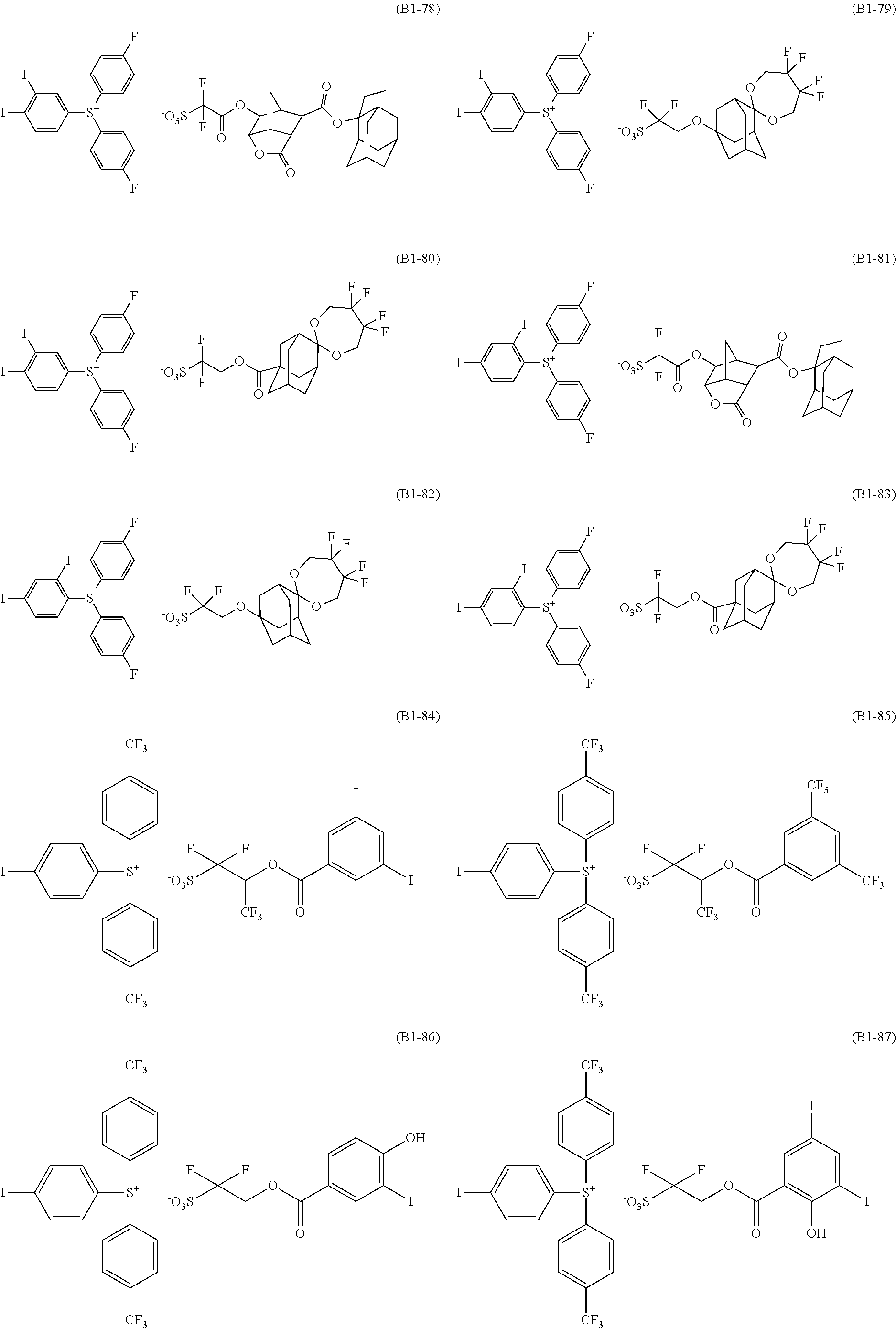

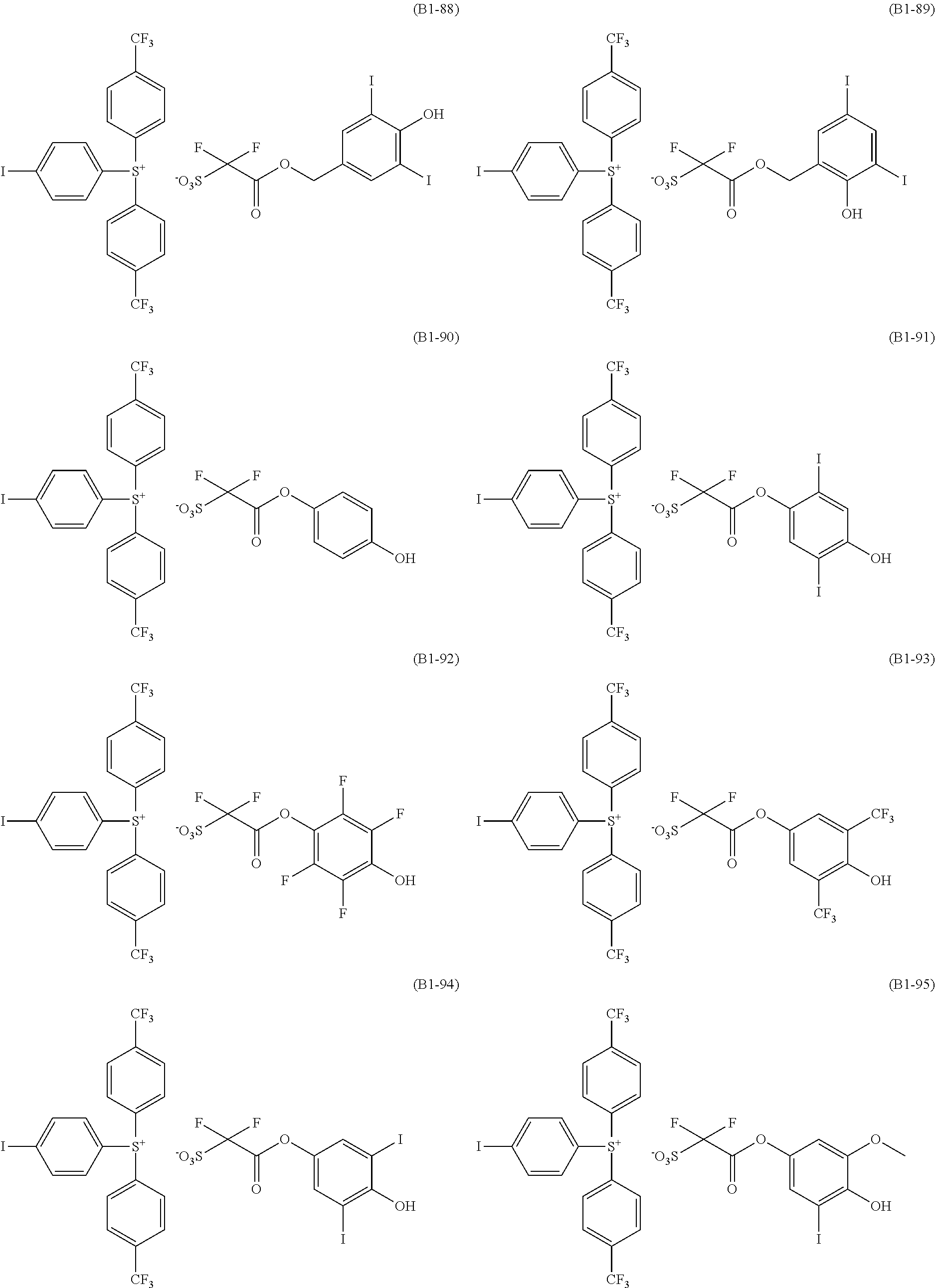

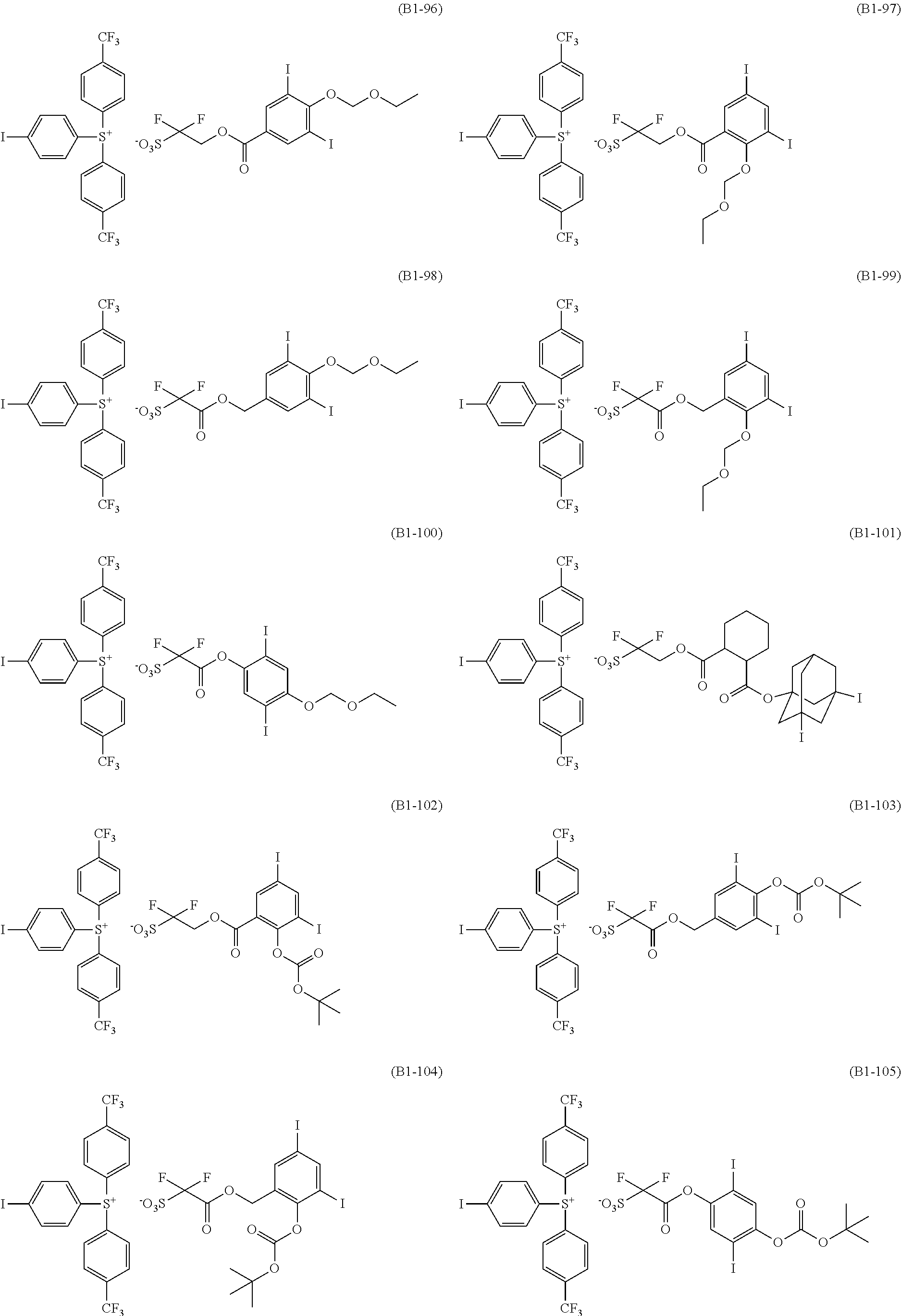

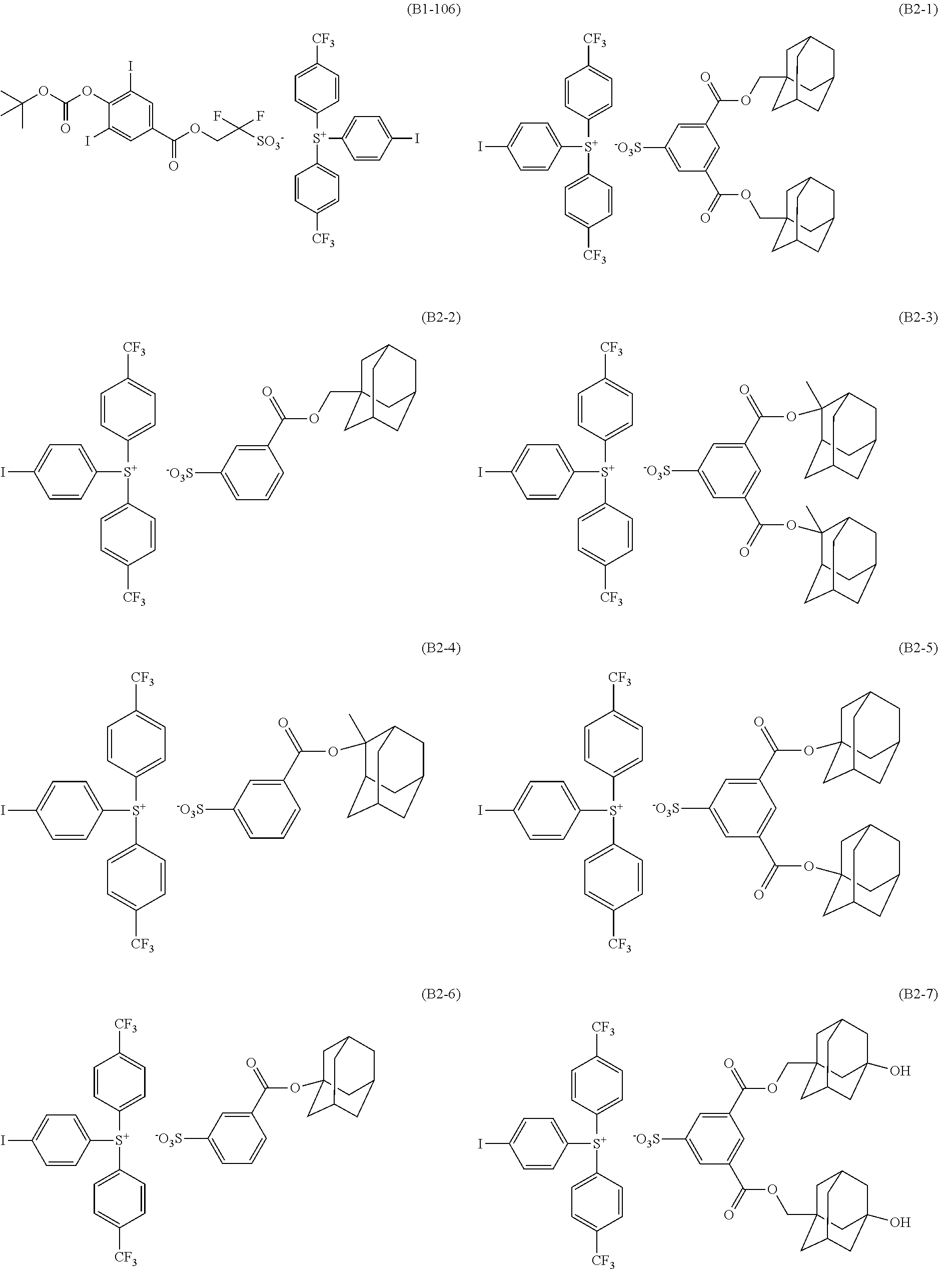

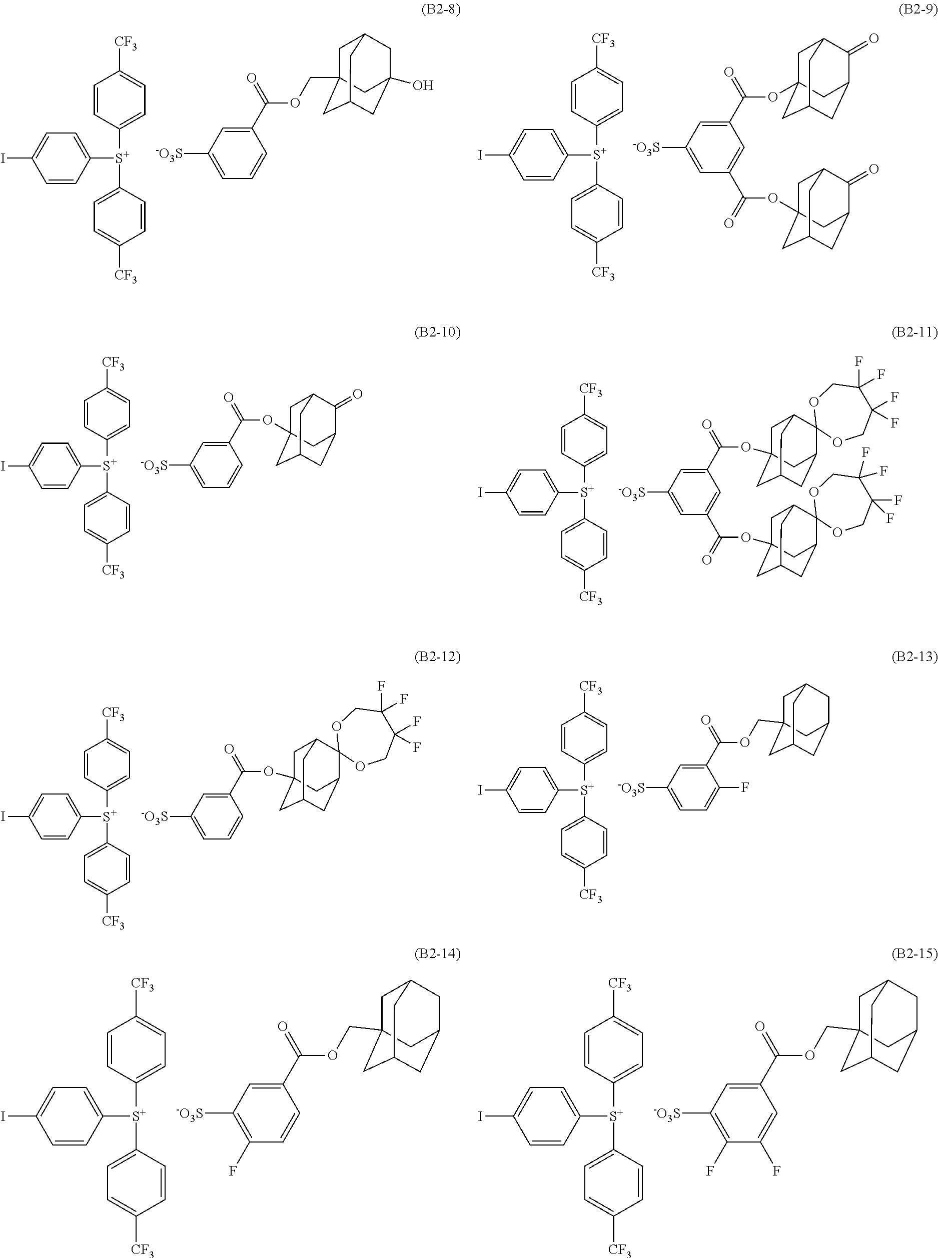

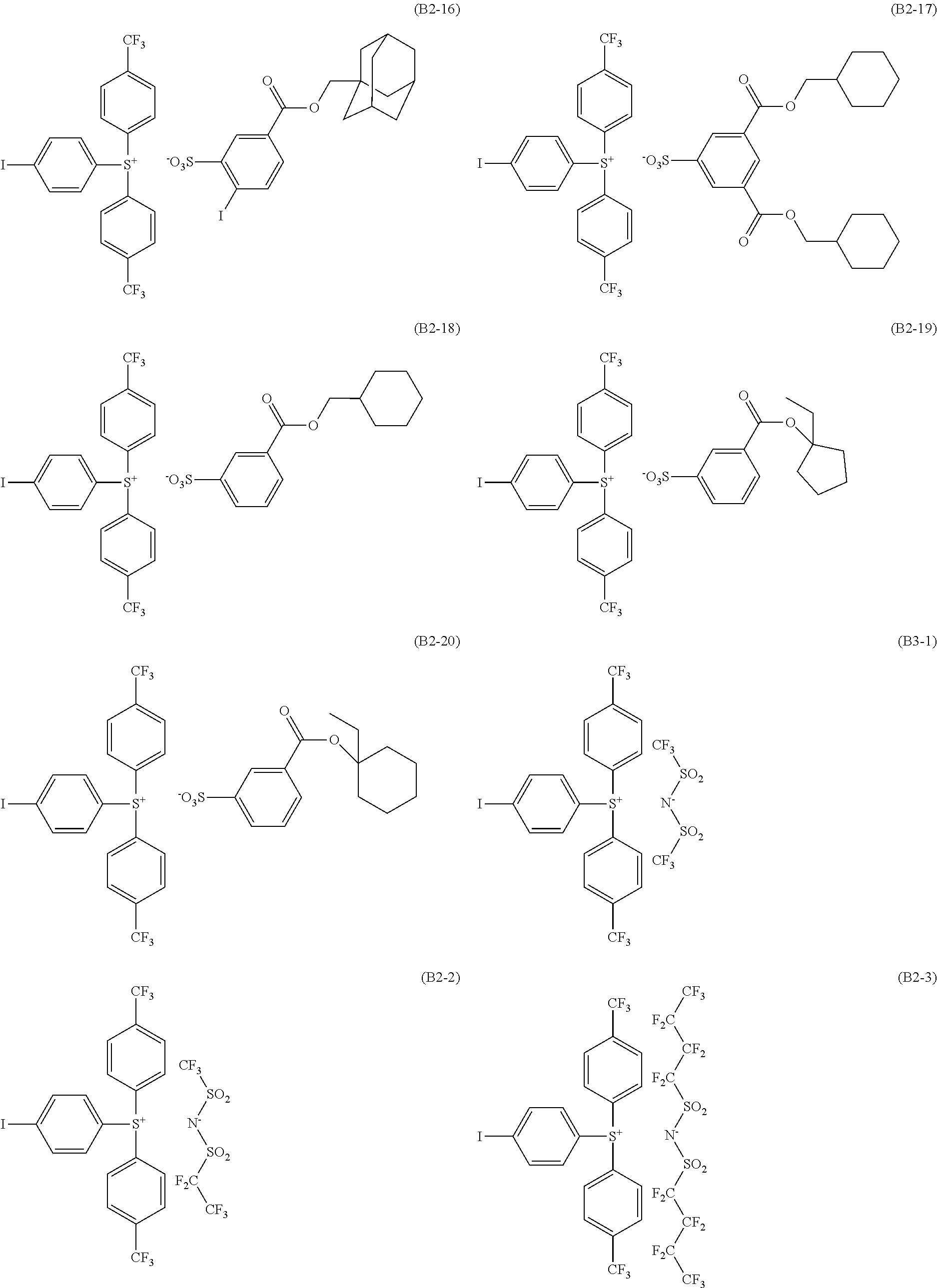

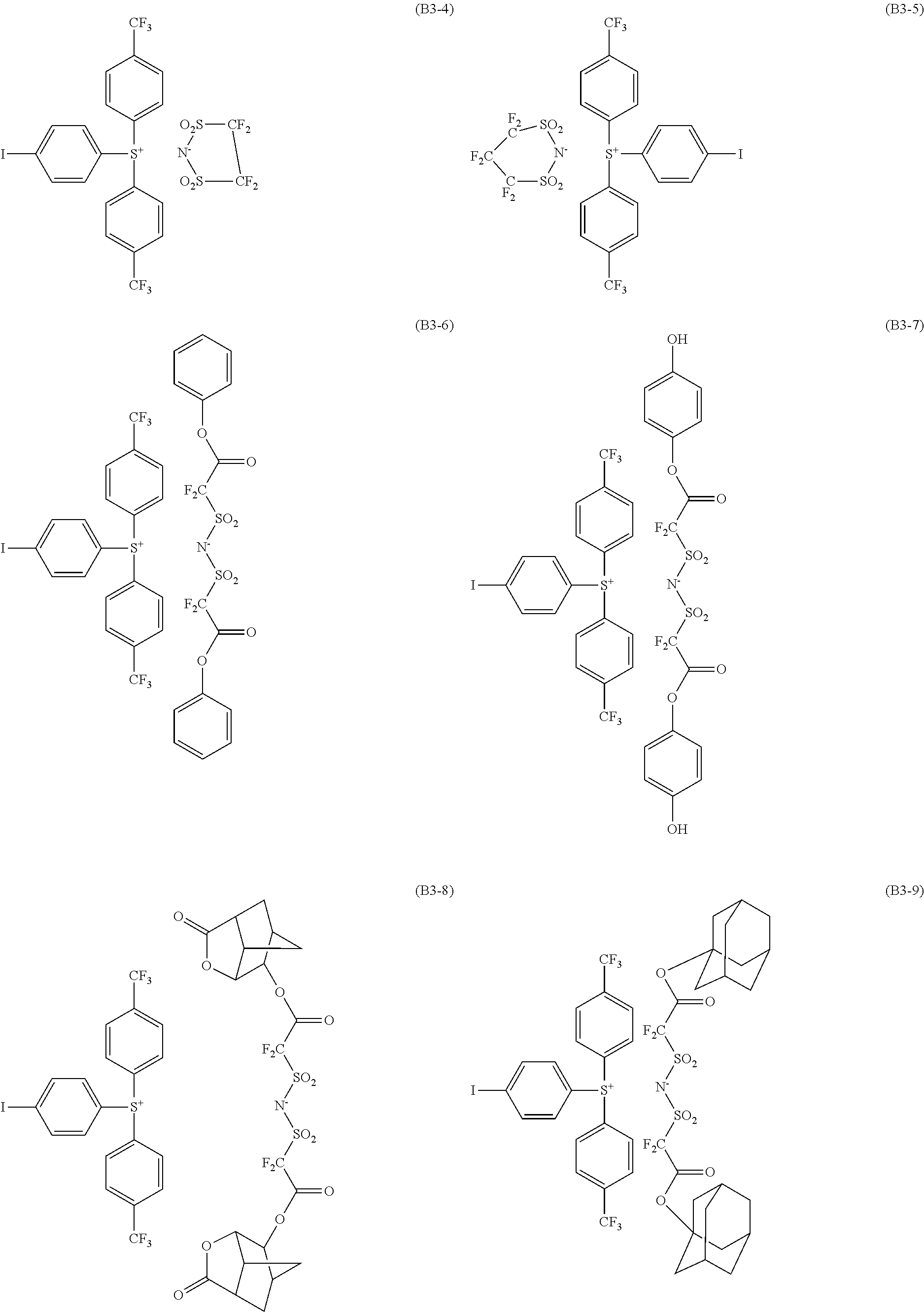

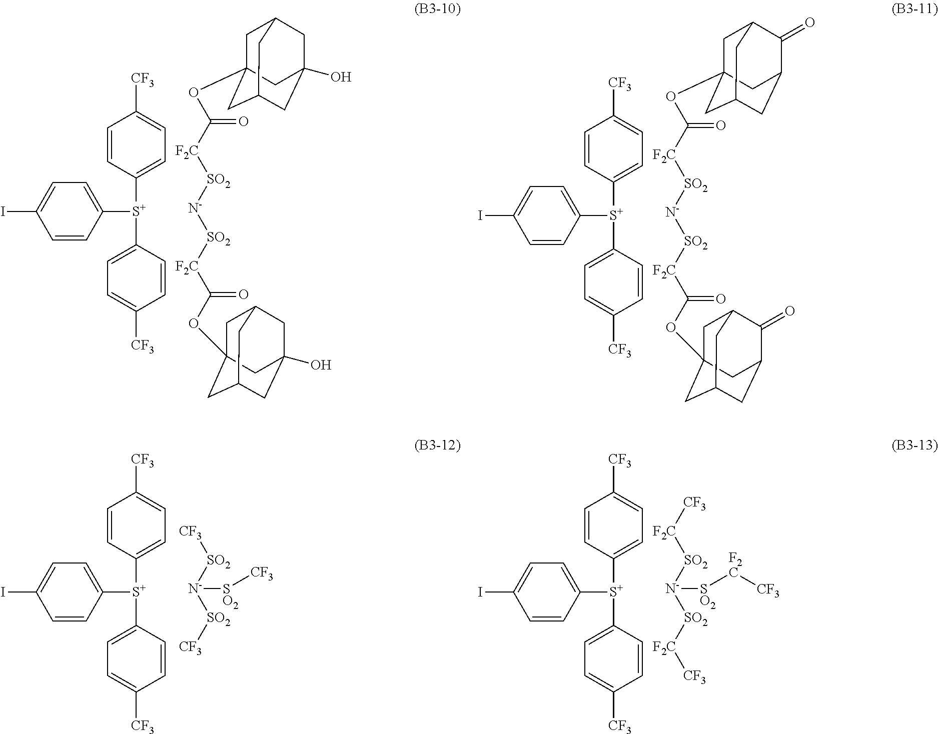

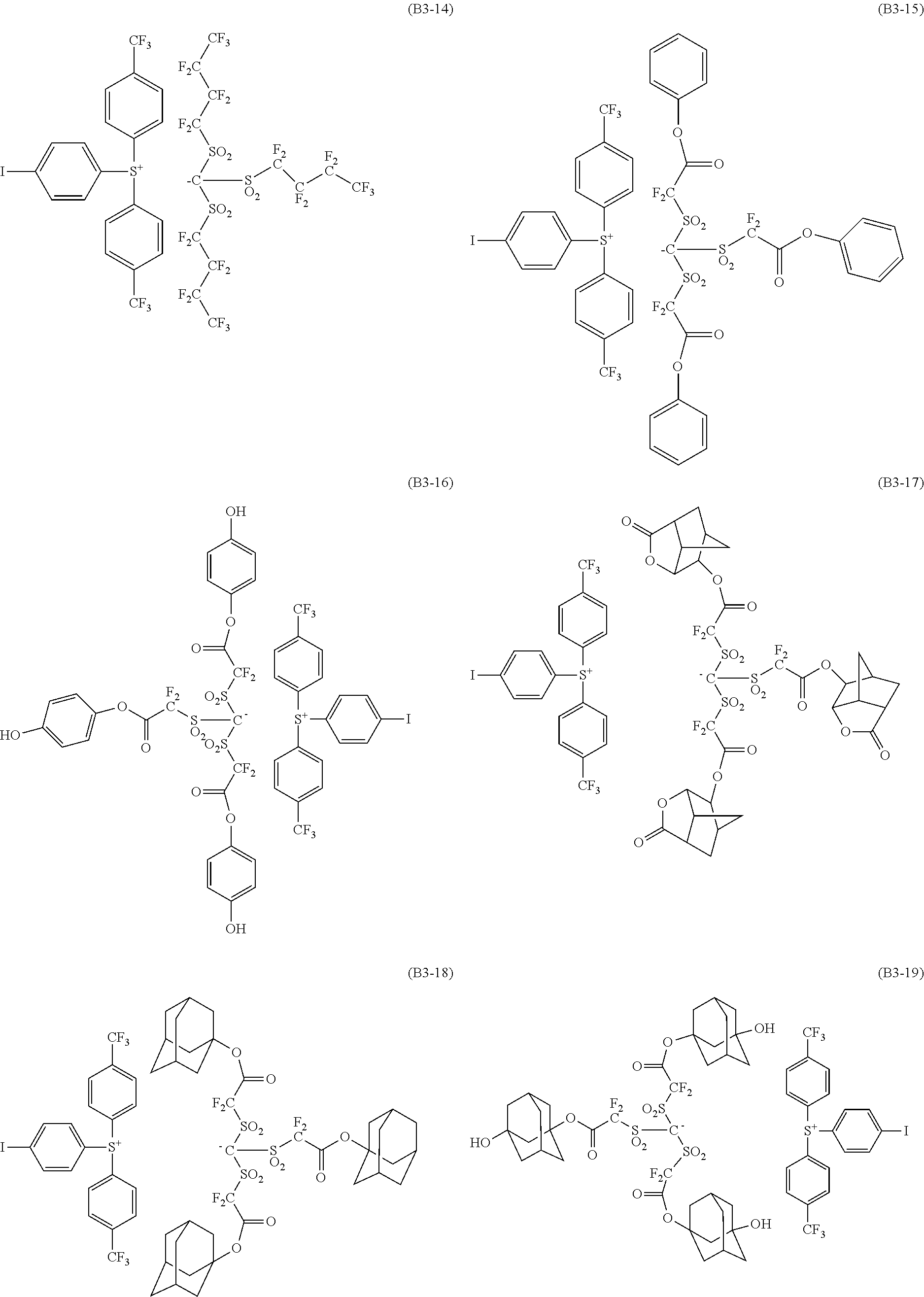

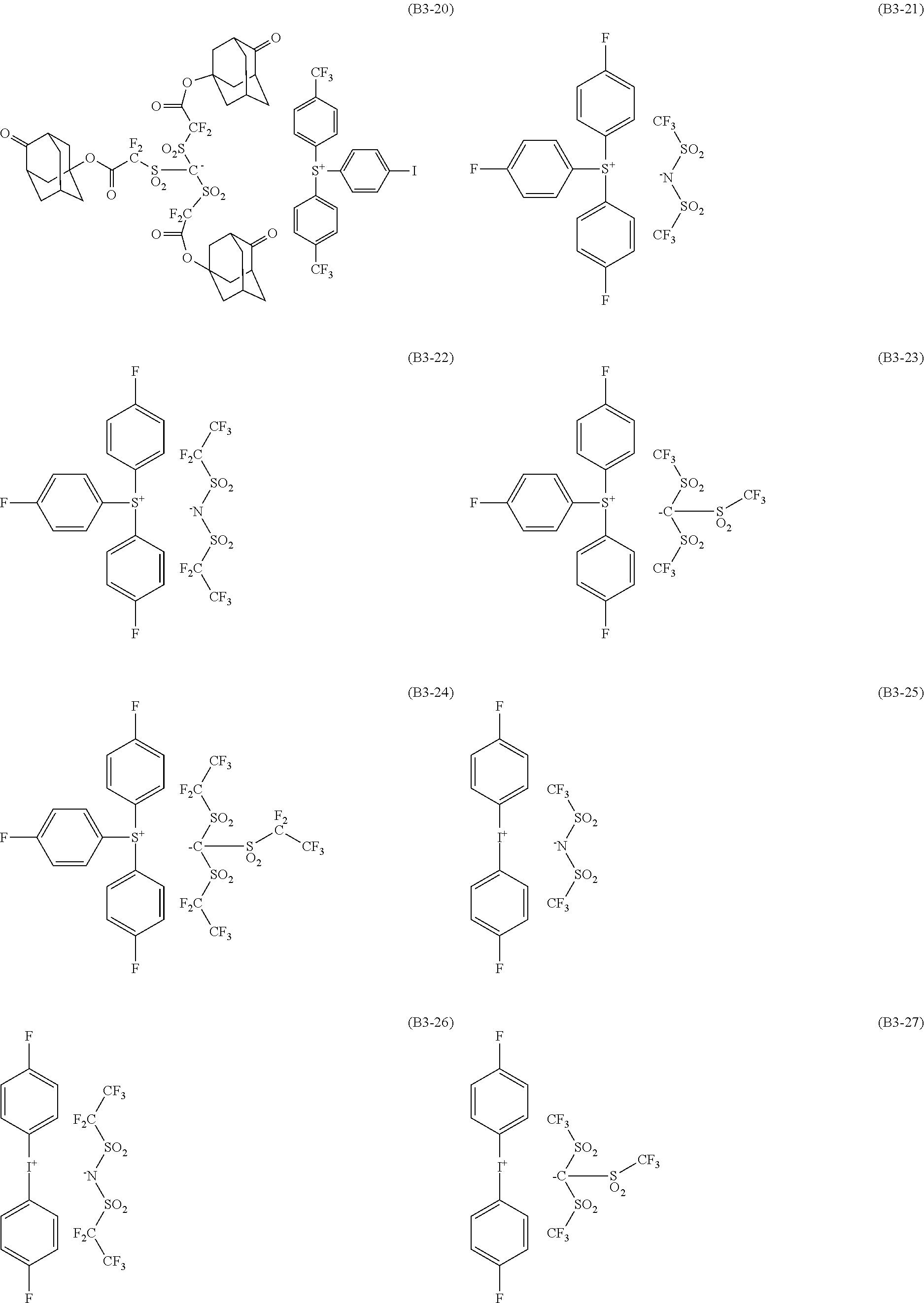

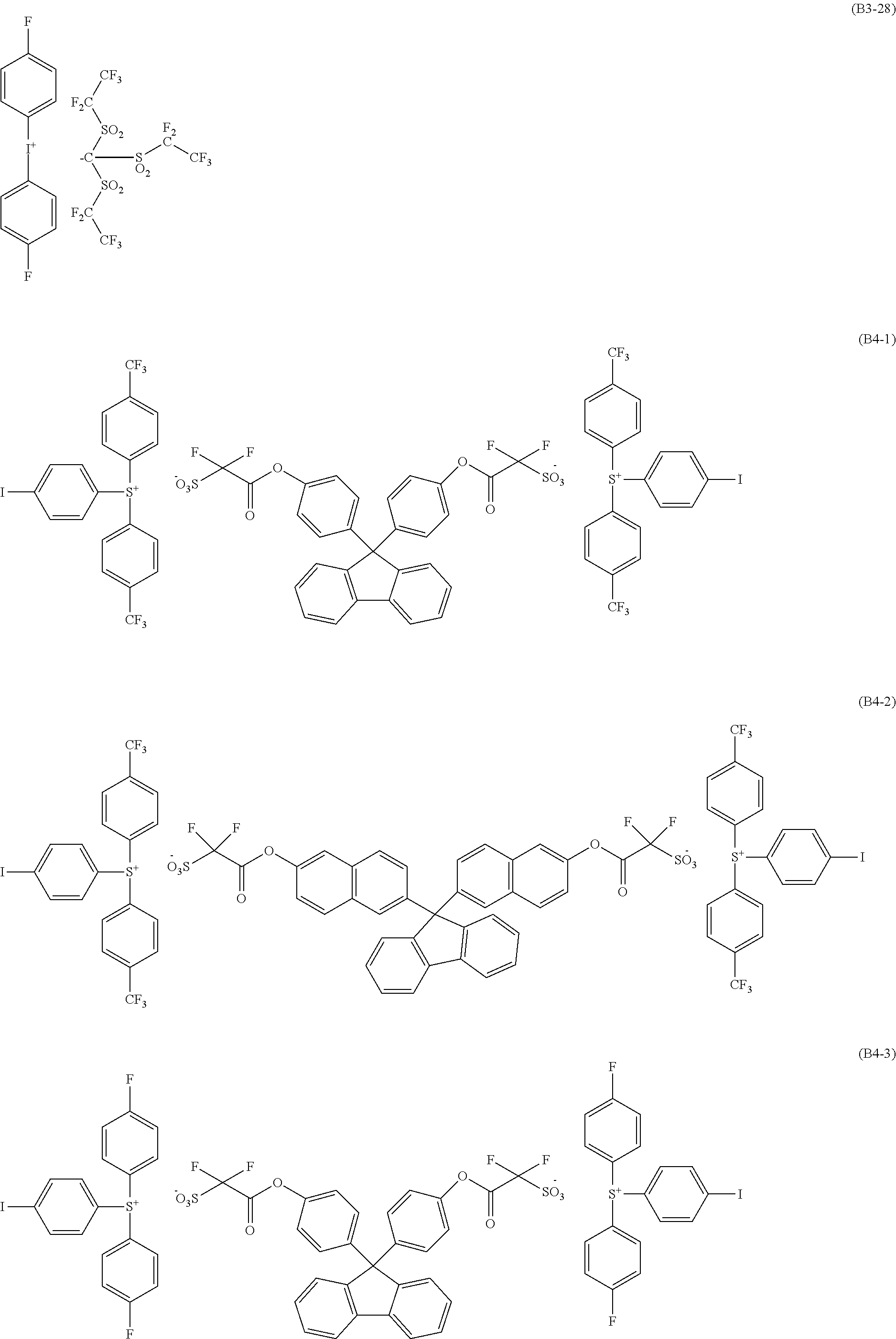

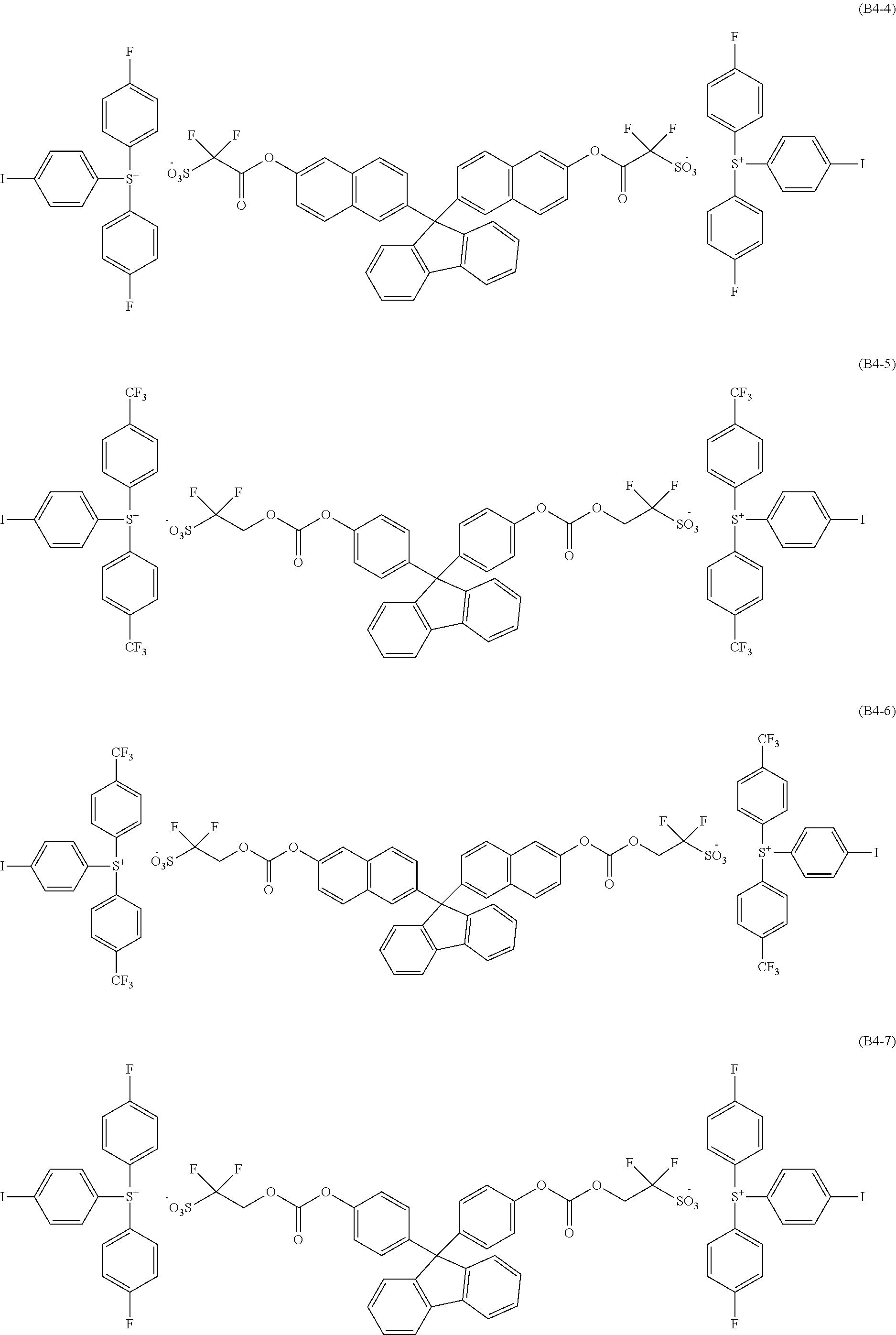

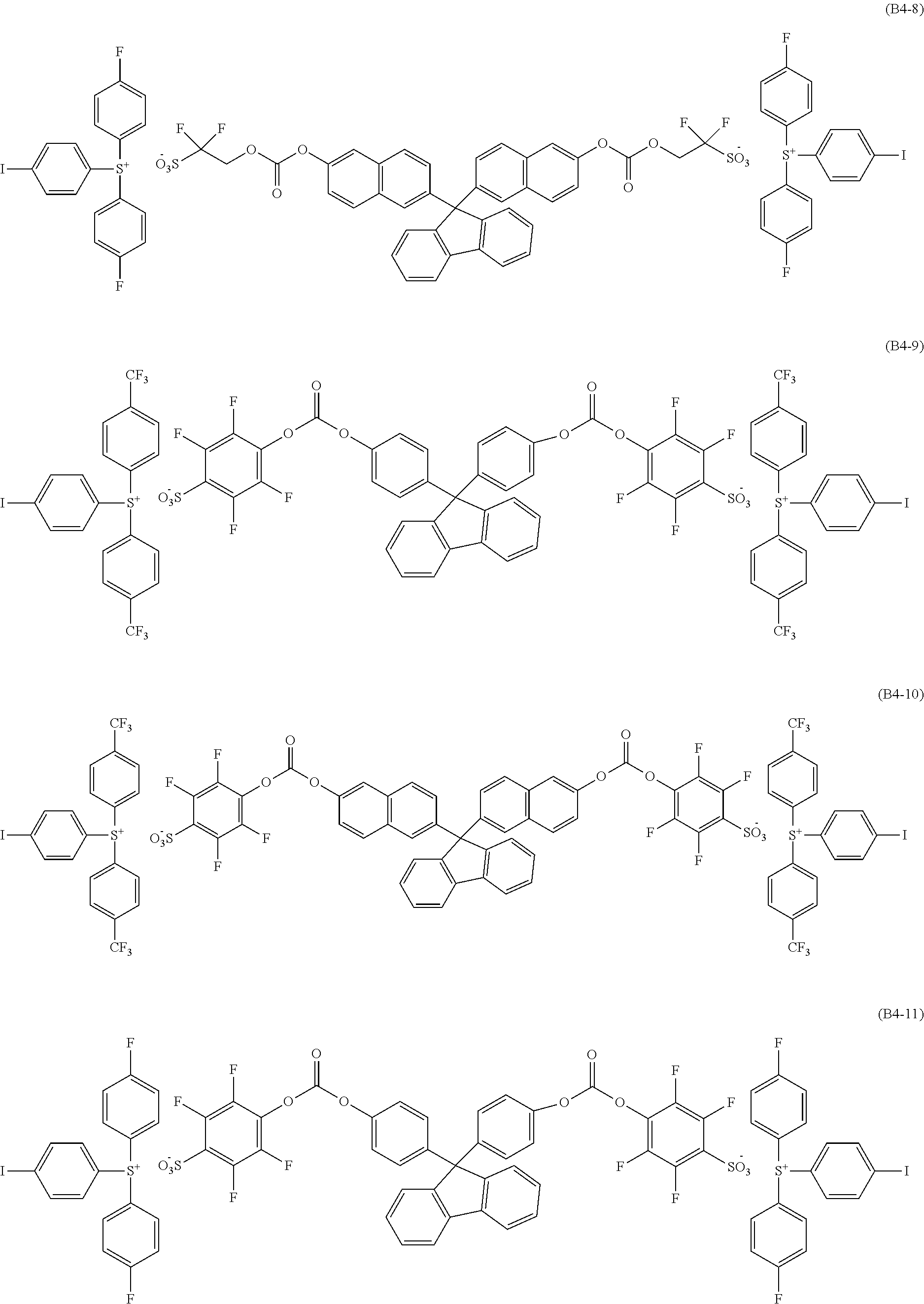

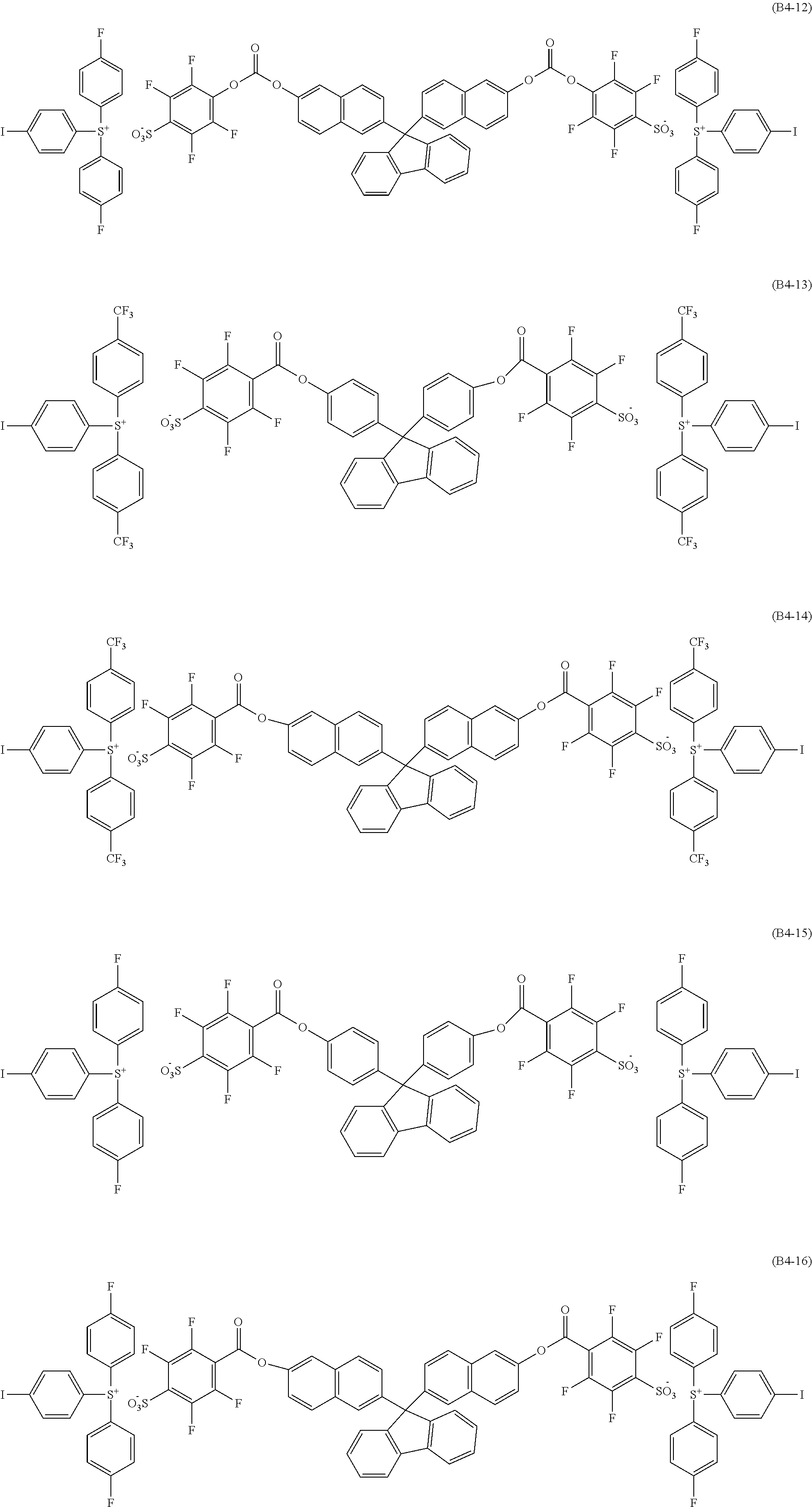

In the resist composition of the present invention, a content ratio of the salt (I) is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and even more preferably 3% by mass or more and 40% by mass or less, with respect to the solid content of the resist composition. When the resist composition contains the resin (A) etc., a content ratio of the salt (I) is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) etc.

In the resist composition of the present invention, a content ratio of structural unit (IP) is preferably 0.1% by mass or more and 99.9% by mass or less, more preferably 1% by mass or more and 99% by mass or less, further preferably 1% by mass or more and 45% by mass or less, even more preferably 1% by mass or more and 40% by mass or less, and even further preferably 3% by mass or more and 40% by mass or less, with respect to the solid content of the resist composition.

In the resist composition of the present invention, a content ratio of the compound (B) is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and even more preferably 3% by mass or more and 40% by mass or less, with respect to the solid content of the resist composition. When the resist composition contains the resin (A) etc., a content ratio of the compound (B) is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) etc.

In the resist composition of the present invention, a total content ratio of the acid generating agent is preferably 0.1% by mass or more and 99.9% by mass or less, more preferably 1% by mass or more and 99% by mass or less, further preferably 1% by mass or more and 45% by mass or less, still more preferably 1% by mass or more and 40% by mass or less, and even more preferably 3% by mass or more and 40% by mass or less, with respect to the solid content of the resist composition. When the resist composition contains the resin (A) etc., a total content ratio of the acid generating agent is preferably 1 part by mass or more and 45 parts by mass or less, more preferably 1 part by mass or more and 40 parts by mass or less, and further preferably 3 parts by mass or more and 40 parts by mass or less, with respect to 100 parts by mass of the resin (A) etc.

### <Solvent (E)>

The content ratio of the solvent (E) in the resist composition is typically 90% by mass or more and 99.9% by mass or less, preferably 92% by mass or more and 99% by mass or less, and more preferably 94% by mass or more and 99% by mass or less. The content ratio of the solvent (E) can be measured by known analytical means such as liquid chromatography or gas chromatography.

Examples of the solvent (E) include: glycol ether esters such as ethyl cellosolve acetate, methyl cellosolve acetate, and propylene glycol monomethyl ether acetate; glycol ethers such as propylene glycol monomethyl ether; esters such as ethyl lactate, butyl acetate, amyl acetate, and ethyl pyruvate; ketones such as acetone, methyl isobutyl ketone, 2-heptanone, and cyclohexanone; cyclic esters such as y-butyrolactone. The solvent (E) may be used singly or in combination of two or more.

### <Quencher (C)>

Examples of the quencher (C) include a salt that generates an acid with weaker acidity than the acid generated from a basic nitrogen-containing organic compound and an acid generating agent (salt (I), structural unit (IP), or compound (B)). When the resist composition includes the quencher (C), the content of the quencher (C) is preferably about 0.01 to 15% by mass, more preferably about 0.01 to 10% by mass, further preferably about 0.1 to 8% by mass, and still more preferably about 0.1 to 7% by mass, with respect to the solid content of the resist composition.

Examples of the basic nitrogen-containing organic compound include an amine and an ammonium salt. Examples of the amine include an aliphatic amine and an aromatic amine. Examples of the aliphatic amine include a primary amine, a secondary amine, and a tertiary amine.

Examples of the amine include 1-naphthylamine, 2-naphthylamine, aniline, diisopropylaniline, 2-, 3-, or 4-methylaniline, 4-nitroaniline, N-methylaniline, N,N-dimethylaniline, diphenylamine, hexylamine, heptylamine, octylamine, nonylamine, decylamine, dibutylamine, dipentylamine, dihexylamine, diheptylamine, dioctylamine, dinonylamine, didecylamine, triethylamine, trimethylamine, tripropylamine, tributylamine, tripentylamine, trihexylamine, triheptylamine, trioctylamine, trinonylamine, tridecylamine, methyldibutylamine, methyldipentylamine, methyldihexylamine, methyldicyclohexylamine, methyldiheptylamine, methyldioctylamine, methyldinonylamine, methyldidecylamine, ethyldibutylamine, ethyldipentylamine, ethyldihexylamine, ethyldiheptylamine, ethyldioctylamine, ethyldinonylamine, ethyldidecylamine, dicyclohexylmethylamine, tris[2-(2-methoxyethoxy)ethyl]amine, triisopropanolamine, ethylenediamine, tetramethylenediamine, hexamethylenediamine, 4,4′-diamino-1,2-diphenylethane, 4,4′-diamino-3,3′-dimethyldiphenylmethane, 4,4′-diamino-3,3′-diethyldiphenylmethane, 2,2′-methylenebisaniline, imidazole, 4-methylimidazole, pyridine, 4-methylpyridine, 1,2-di(2-pyridyl)ethane, 1,2-di(4-pyridyl)ethane, 1,2-di(2-pyridyl)ethene, 1,2-di(4-pyridyl)ethene, 1,3-di(4-pyridyl)propane, 1,2-di(4-pyridyloxy)ethane, di(2-pyridyl)ketone, 4,4′-dipyridyl sulfide, 4,4′-dipyridyl disulfide, 2,2′-dipyridylamine, 2,2′-dipicolylamine, and bipyridine, preferably, diisopropylaniline, and more preferably, 2,6-diisopropylaniline.

Examples of the ammonium salt include tetramethylammonium hydroxide, tetraisopropylammonium hydroxide, tetrabutylammonium hydroxide, tetrahexylammonium hydroxide, tetraoctylammonium hydroxide, phenyltrimethylammonium hydroxide, 3-(trifluoromethyl)phenyltrimethylammonium hydroxide, tetra-n-butylammonium salicylate, and choline.

The acidity of a salt that generates an acid having a weaker acidity than the acid generated from an acid generating agent is indicated by the acid dissociation constant (pKa). The salt that generates an acid having a weaker acidity than the acid generated from an acid generating agent is typically a salt with an acid dissociation constant of the acid generated from the salt being −3<pKa, preferably −1<pKa<7, and more preferably 0<pKa<5.

Examples of the salt that generates an acid with a weaker acidity than the acid generated from the acid generating agent include salts represented by the following formula, salts represented by formula (D) disclosed in Japanese Patent Laid-Open No. 2015-147926 (hereinafter, may be referred to as “weak acid inner salt (D)”), and salts disclosed in Japanese Patent Laid-Open No. 2012-229206, Japanese Patent Laid-Open No. 2012-6908, Japanese Patent Laid-Open No. 2012-72109, Japanese Patent Laid-Open No. 2011-39502, and Japanese Patent Laid-Open No. 2011-191745. The salt that generates an acid weaker in acidity than the acid generated from the acid generating agent is preferably a salt that generates a carboxylic acid weaker in acidity than the acid generated from the acid generating agent (a salt having a carboxylate anion), more preferably a weak acid inner salt (D), and further preferably a diphenyliodonium salt including a phenyl group substituted with a carboxylate anion among the weak acid inner salts (D).

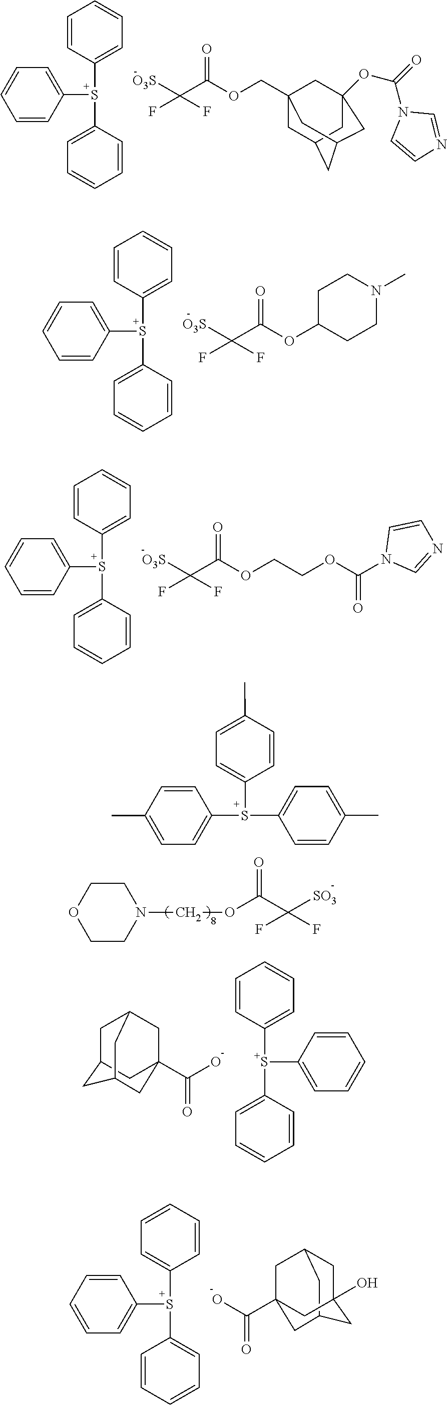

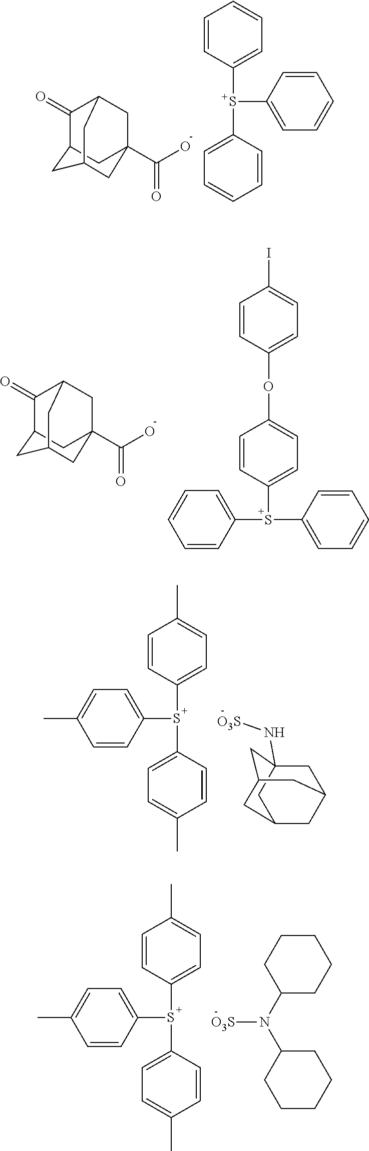

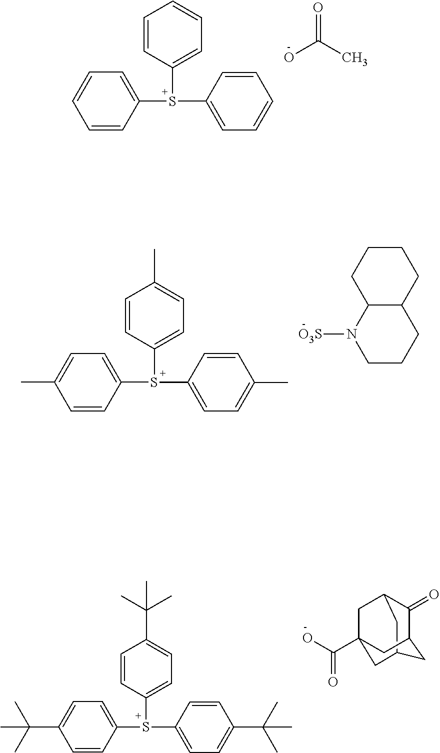

As the weak acid inner salt (D), a diphenyliodonium salt having an iodonium cation to which two phenyl groups are bonded and a carboxylate anion substituted for at least one of the two phenyl groups bonded to the iodonium cation is preferable, specifically, a salt represented by the following formula.

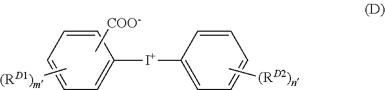

wherein in formula (D)
R^{D1} and R^{D2} each independently represent a hydrocarbon group having 1 to 12 carbon atoms, an alkoxy group having 1 to 6 carbon atoms, an acyl group having 2 to 7 carbon atoms, an acyloxy group having 2 to 7 carbon atoms, an alkoxycarbonyl group having 2 to 7 carbon atoms, a nitro group, or a halogen atom; and
m′ and n′ each independently represent an integer of 0 to 4, and when m′ is 2 or more, a plurality of R^{D1} are the same or different from each other, and when n′ is 2 or more, a plurality of R^{D2} may be the same or different from each other.

Examples of the hydrocarbon group of R^{D1} and R^{D2} include a chain hydrocarbon group, an alicyclic hydrocarbon group, an aromatic hydrocarbon group, and a group formed by combining these groups.

Examples of the chain hydrocarbon group include an alkyl group such as a methyl group, an ethyl group, a propyl group, an isopropyl group, a butyl group, an isobutyl group, a tert-butyl group, a pentyl group, a hexyl group, and a nonyl group.

The alicyclic hydrocarbon group may be either monocyclic or polycyclic, and may be either saturated or unsaturated. Examples thereof include a cycloalkyl group such as a cyclopropyl group, a cyclobutyl group, a cyclopentyl group, a cyclohexyl group, a cyclononyl group, and a cyclododecyl group, as well as a norbornyl group and an adamantyl group.

Examples of the aromatic hydrocarbon group include an aryl group such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, an anthryl group, a p-adamantylphenyl group, a tolyl group, a xylyl group, a cumenyl group, a mesityl group, a biphenyl group, a phenanthryl group, a 2,6-diethylphenyl group, and a 2-methyl-6-ethylphenyl group.

Examples of the group formed by combining these groups include an alkyl-cycloalkyl group, a cycloalkyl-alkyl group, and an aralkyl group (e.g., a phenylmethyl group, a 1-phenylethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group, and a 6-phenyl-1-hexyl group).

Examples of the alkoxy group include a methoxy group and an ethoxy group.

Examples of the acyl group include an acetyl group, a propanoyl group, a benzoyl group, and a cyclohexanecarbonyl group.

Examples of the acyloxy group include the group in which an oxy group (—O—) is bonded to the above-described acyl group.

Examples of the alkoxycarbonyl group include a group in which a carbonyl group (—CO—) is bonded to the above-described alkoxy group.

Examples of the halogen atom include a fluorine atom, a chlorine atom, and a bromine atom.

R^{D1} and R^{D2} are each independently preferably an alkyl group having 1 to 8 carbon atoms, a cycloalkyl group having 3 to 10 carbon atoms, an alkoxy group having 1 to 6 carbon atoms, an acyl group having 2 to 4 carbon atoms, an acyloxy group having 2 to 4 carbon atoms, an alkoxycarbonyl group having 2 to 4 carbon atoms, a nitro group, or a halogen atom.

m′ and n′ are each independently preferably an integer of 0 to 2, and more preferably 0. When m′ is 2 or more, a plurality of R^{D1} are the same or different from each other, and when n′ is 2 or more, a plurality of R^{D2} may be the same or different from each other.

More specific examples thereof include the following salts.

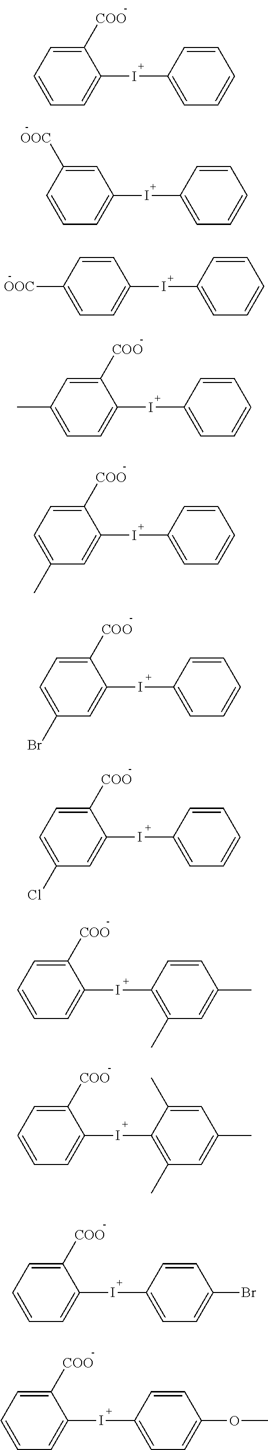

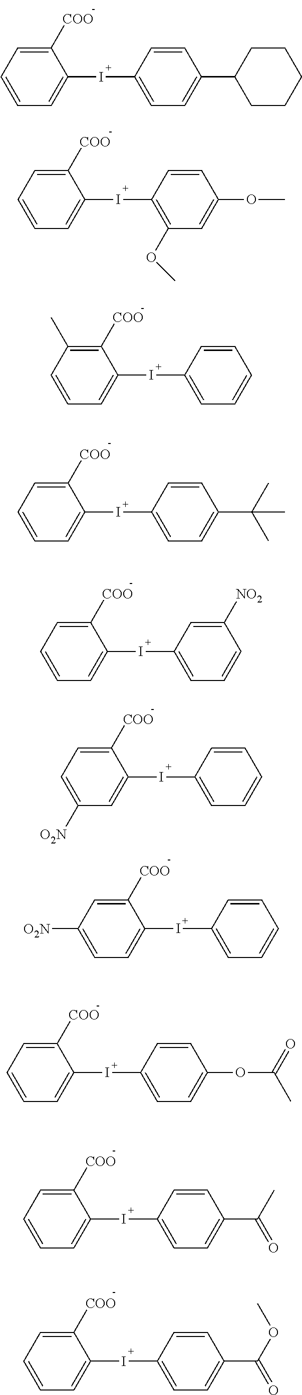

### <Other Components>

The resist composition of the present invention may contain components other than those described above (hereinafter, may be referred to as “other components (F)”) if necessary. The other components (F) are not particularly limited, and an additive known in the resist field, such as a sensitizer, a dissolution inhibitor, a surfactant, a stabilizer, and a dye can be used.

### <Preparation of Resist Composition>

The resist composition of the present invention can be prepared by mixing the salt (I) or a compound or resin that contains the structural unit (IP), and, if necessary, the resin (A) etc., the compound (B), a resin other than the resin (A) etc., the solvent (E), the quencher (C), and other components (F). The order of mixing is arbitrary and is not particularly limited. For the temperature during mixing, a suitable temperature can be selected from 10 to 40° C. depending on, for example, the type of resin and the like, the solubility of the resin and the like in the solvent (E). For the mixing time, a suitable time can be selected from 0.5 to 24 hours depending on the mixing temperature. The mixing means is not particularly limited, and stirring and mixing can be used.

After mixing the components, it is preferable to filter the mixture using a filter with a pore size of about 0.003 to 0.2 μm.

### <Method for Producing Resist Pattern>

The method for producing a resist pattern of the present invention includes:
(1) a step of applying the resist composition of the present invention onto a substrate;
(2) a step of drying the applied resist composition to form a composition layer;
(3) a step of exposing the composition layer;
(4) a step of heating the exposed composition layer; and
(5) a step of developing the heated composition layer.

The resist composition can be applied onto a substrate using a commonly used device such as a spin coater. Examples of the substrate include an inorganic substrate such as a silicon wafer, and an organic substrate with a resist film or the like formed on the surface thereof. Before applying the resist composition, the substrate may be cleaned, and an anti-reflective film or the like may be formed on the substrate.

The applied composition is dried to remove the solvent and form a composition layer. Drying is performed, for example, by evaporating the solvent using a heating device such as a hot plate (so-called pre-baking), or by using a vacuum device. The heating temperature is preferably 50 to 200° C., and the heating time is preferably 10 to 180 seconds. The pressure during vacuum drying is preferably about 1 to 1.0×10⁵ Pa.

The resulting composition layer is typically exposed using an exposure machine. The exposure machine may be an immersion exposure machine. Various types of exposure light sources can be used, including those that emit ultraviolet laser light such as KrF excimer laser (wavelength 248 nm), ArF excimer laser (wavelength 193 nm), and F₂ excimer laser (wavelength 157 nm), those that convert the wavelength of laser light from a solid-state laser light source (YAG or semiconductor laser) to emit harmonic laser light in the far ultraviolet or vacuum ultraviolet range, and those that irradiate with electron beams or extreme ultraviolet light (EUV). In this specification, irradiation with these types of radiation may be collectively referred to as “exposure”. Exposure is typically performed through a mask corresponding to the desired pattern. If the exposure light source is an electron beam, exposure may be performed by direct drawing without using a mask.

The exposed composition layer is subjected to a heat treatment (so-called post-exposure bake) in order to promote the deprotection reaction of the acid labile groups. The heating temperature is typically about 50 to 200° C., preferably about 70 to 150° C. A chemical treatment (silylation) may be performed to adjust the hydrophilicity or hydrophobicity of the resin on the surface side of the heated composition. In addition, the steps of applying a resist composition, drying, exposing, and heating may be repeated on the exposed composition layer before development.

The heated composition layer is typically developed using a developing device with a developer. Examples of the development method include the dipping method, the paddle method, the spray method, and the dynamic dispensing method. The development temperature is preferably, for example, 5 to 60° C., and the development time is preferably, for example, 5 to 300 seconds. Selecting the type of developer as follows allows a positive resist pattern or a negative resist pattern to be produced.

When a positive resist pattern is produced from the resist composition of the present invention, an alkaline developer is used as the developer. The alkaline developer may be any of various alkaline aqueous solutions used in this field. Examples thereof include an aqueous solution of tetramethylammonium hydroxide and (2-hydroxyethyl)trimethylammonium hydroxide (commonly known as choline). The alkaline developer may include a surfactant.

After development, it is preferable to wash the resist pattern with ultrapure water, and then remove any water remaining on the substrate and pattern.

When a negative resist pattern is produced from the resist composition of the present invention, a developer including an organic solvent (hereinafter may be referred to as an “organic developer”) is used as the developer.

Examples of the organic solvent included in the organic developer include: a ketone solvent such as 2-hexanone and 2-heptanone; a glycol ether ester solvent such as propylene glycol monomethyl ether acetate; an ester solvent such as butyl acetate; a glycol ether solvent such as propylene glycol monomethyl ether; an amide solvent such as N,N-dimethylacetamide; and an aromatic hydrocarbon solvent such as anisole.

The content ratio of the organic solvent in the organic developer is preferably 90% by mass or more and 100% by mass or less, more preferably 95% by mass or more and 100% by mass or less, and further preferably substantially only the organic solvent.

Among these, preferable organic developers are those including butyl acetate and/or 2-heptanone. The total content ratio of butyl acetate and 2-heptanone in the organic developer is preferably 50% by mass or more and 100% by mass or less, more preferably 90% by mass or more and 100% by mass or less, and further preferably substantially only butyl acetate and/or 2-heptanone.

The organic developer may include a surfactant. The organic developer may also include a small amount of water.

During development, development may be stopped by replacing the organic developer with a different type of solvent.

It is preferable to rinse the developed resist pattern with a rinse solution. The rinse solution is not particularly limited as long as it does not dissolve the resist pattern, and a solution including a general organic solvent can be used, preferably an alcohol solvent or an ester solvent.

After cleaning, it is preferable to remove the rinse solution remaining on the substrate and the pattern.

### <Applications>

The resist composition of the present invention is suitable as a resist composition for KrF excimer laser exposure, a resist composition for ArF excimer laser exposure, a resist composition for electron beam (EB) exposure, or a resist composition for EUV exposure, and is particularly suitable as a resist composition for electron beam (EB) exposure or a resist composition for EUV exposure, and is useful for semiconductor microfabrication.

### EXAMPLES

The present invention will be explained in more detail with reference to examples. In the examples, “%” and “parts” that indicate the content or amount used are based on mass unless otherwise specified.

The weight average molecular weight is a value determined by gel permeation chromatography. The analytical conditions for gel permeation chromatography are as follows.
Column: TSKgel Multipore HXL-M×3+guardcolumn (manufactured by Tosoh Corporation)
Eluent: tetrahydrofuran
Flow rate: 1.0 mL/min
Detector: RI detector
Column temperature: 40° C.
Injection volume: 100 μl
Molecular weight standard: Standard polystyrene (manufactured by Tosoh Corporation)

The structure of the compound was confirmed by measuring the molecular ion peak using mass spectrometry (LC: 1100 model manufactured by Agilent Technologies, Inc., MASS: LC/MSD model manufactured by Agilent Technologies, Inc.). In the following examples, the value of this molecular ion peak is indicated by “MASS”.

### Example 1: Synthesis of Salt Represented by Formula (I-1)

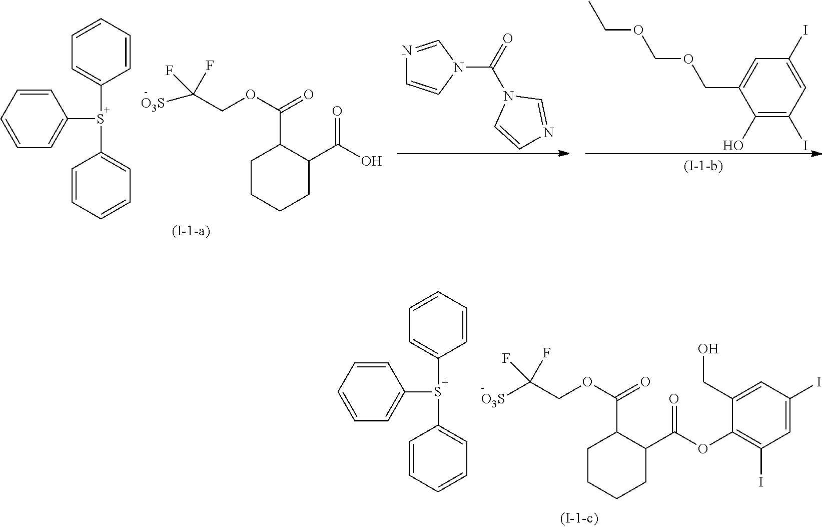

5.79 parts of the salt represented by formula (I-1-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.08 parts of the salt represented by formula (I-1-c).

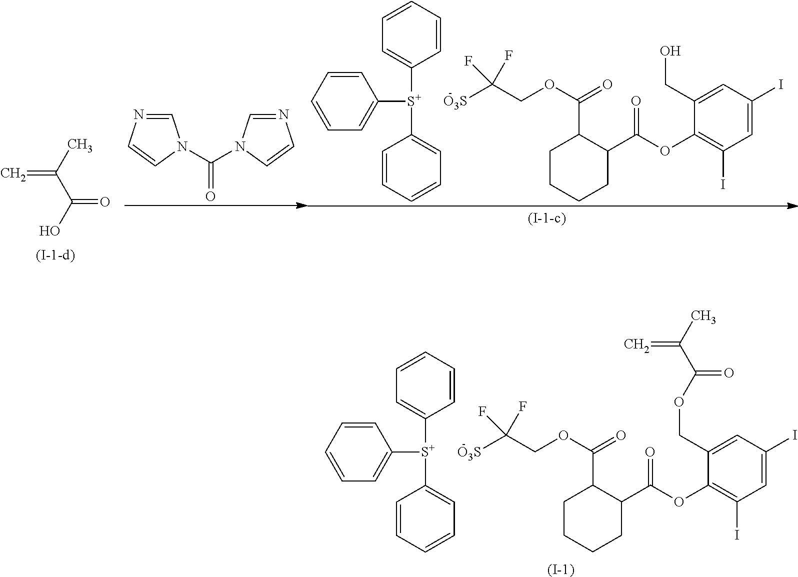

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 4.68 parts of the salt represented by formula (I-1-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 4.53 parts of the salt represented by formula (I-1).

MS (ESI (+) Spectrum): M⁺ 263.1

MS (ESI (−) Spectrum): M⁻ 740.9

### Example 2: Synthesis of Salt Represented by Formula (I-2)

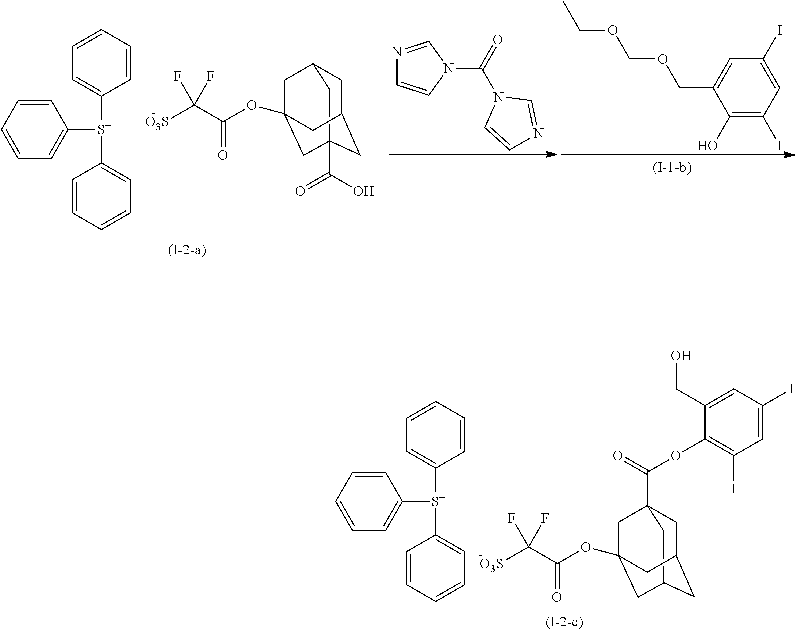

6.17 parts of the salt represented by formula (I-2-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.55 parts of the salt represented by formula (I-2-c).

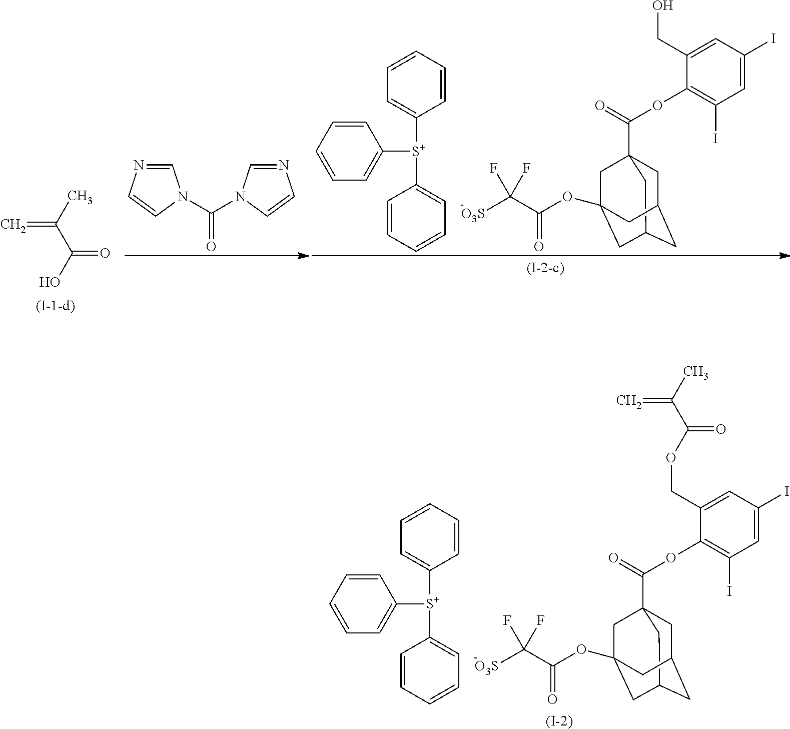

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 4.87 parts of the salt represented by formula (I-2-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 6.63 parts of the salt represented by formula (I-2).

MS (ESI (+) Spectrum): M⁺ 263.1

MS (ESI (−) Spectrum): M⁻ 778.9

### Example 3: Synthesis of Salt Represented by Formula (I-4)

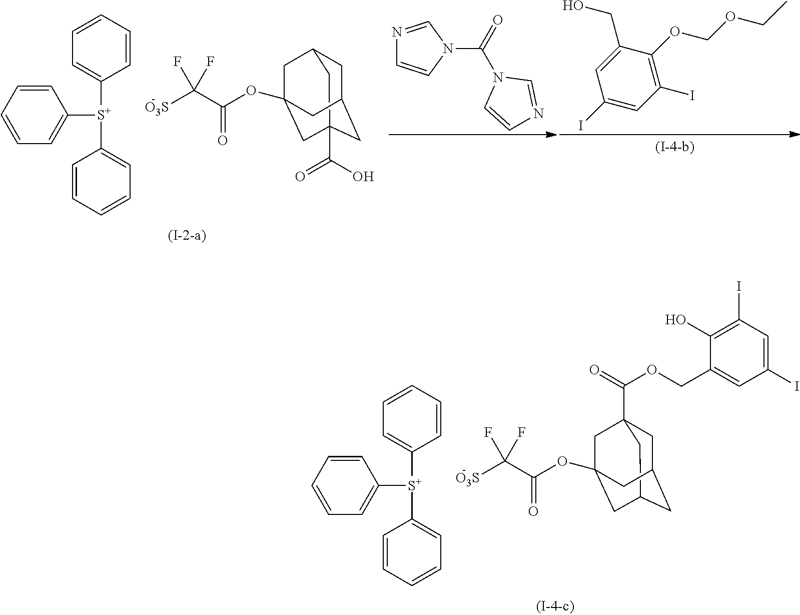

6.17 parts of the salt represented by formula (I-2-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-4-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.96 parts of the salt represented by formula (I-4-c).

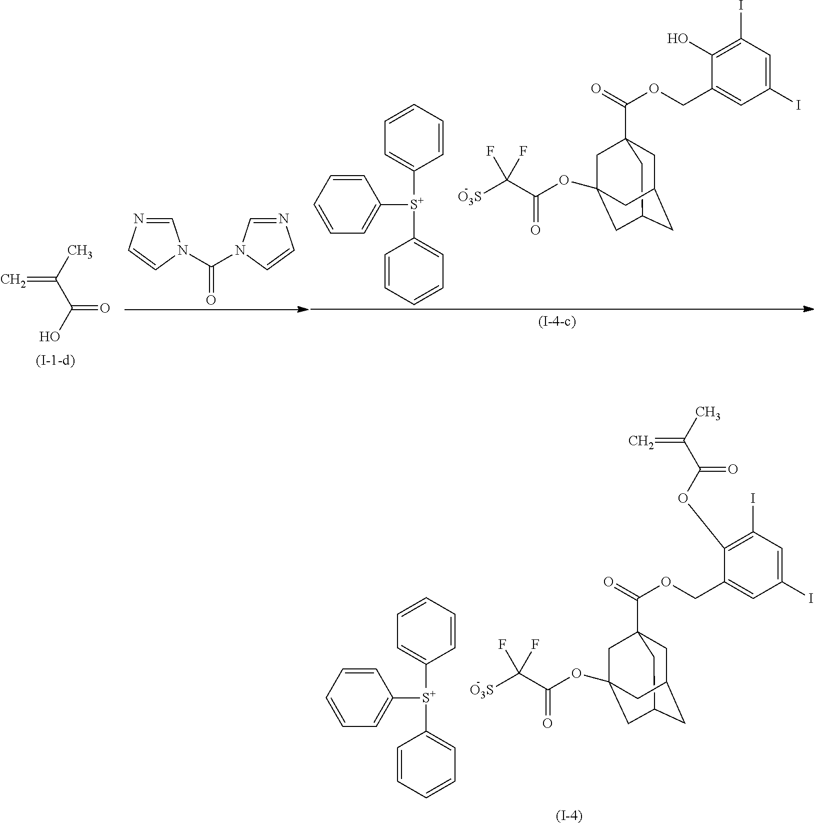

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 4.87 parts of the salt represented by formula (I-4-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 4.94 parts of the salt represented by formula (I-4).

MS (ESI (+) Spectrum): M⁺ 263.1

MS (ESI (−) Spectrum): M⁻ 778.9

### Example 4: Synthesis of Salt Represented by Formula (I-2501)

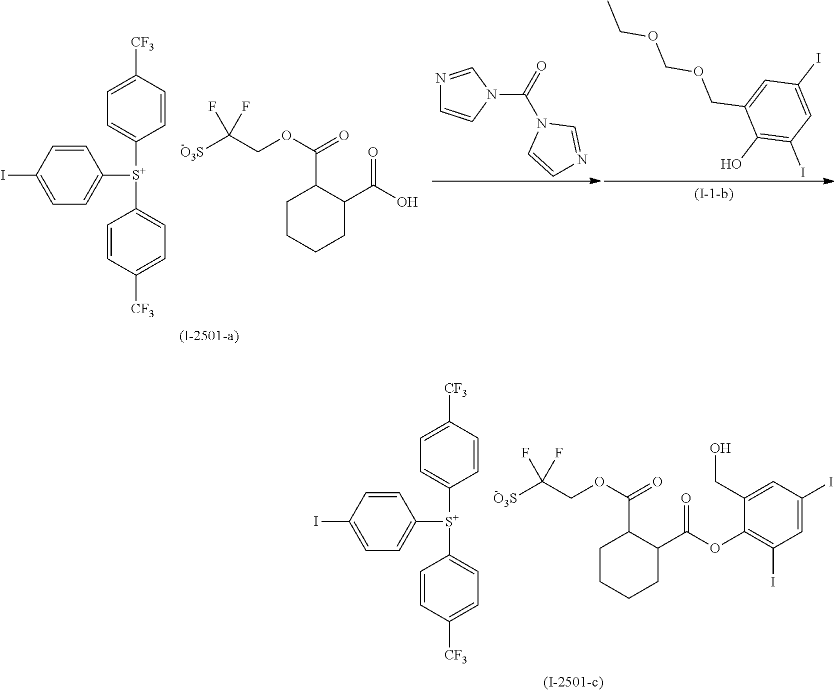

8.41 parts of the salt represented by formula (I-2501-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.91 parts of the salt represented by formula (I-2501-c).

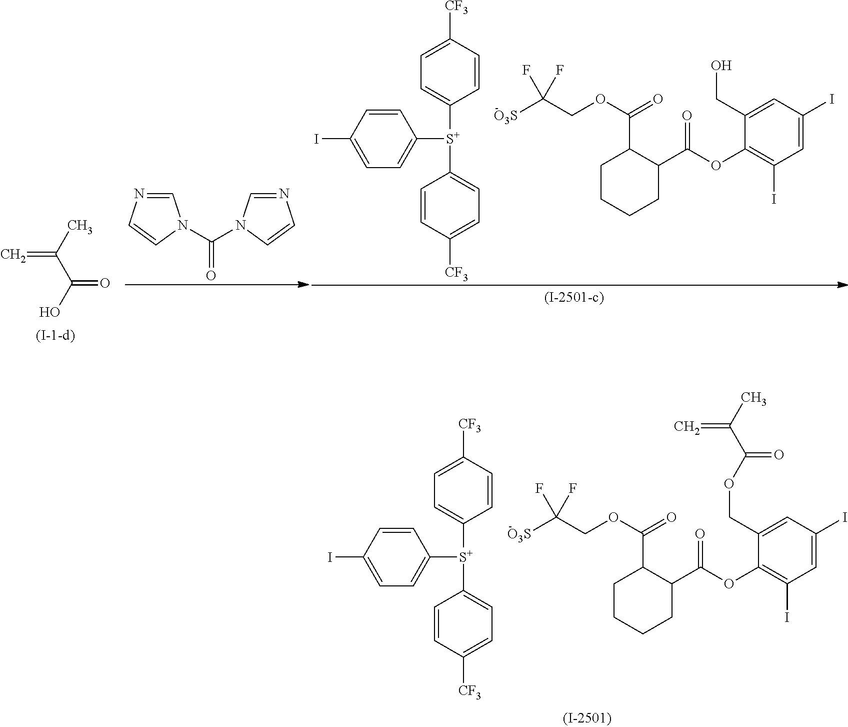

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 5.99 parts of the salt represented by formula (I-2501-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 5.75 parts of the salt represented by formula (I-2501).

MS (ESI (+) Spectrum): M⁺ 525.0

MS (ESI (−) Spectrum): M⁻ 740.9

### Example 5: Synthesis of Salt Represented by Formula (I-2401)

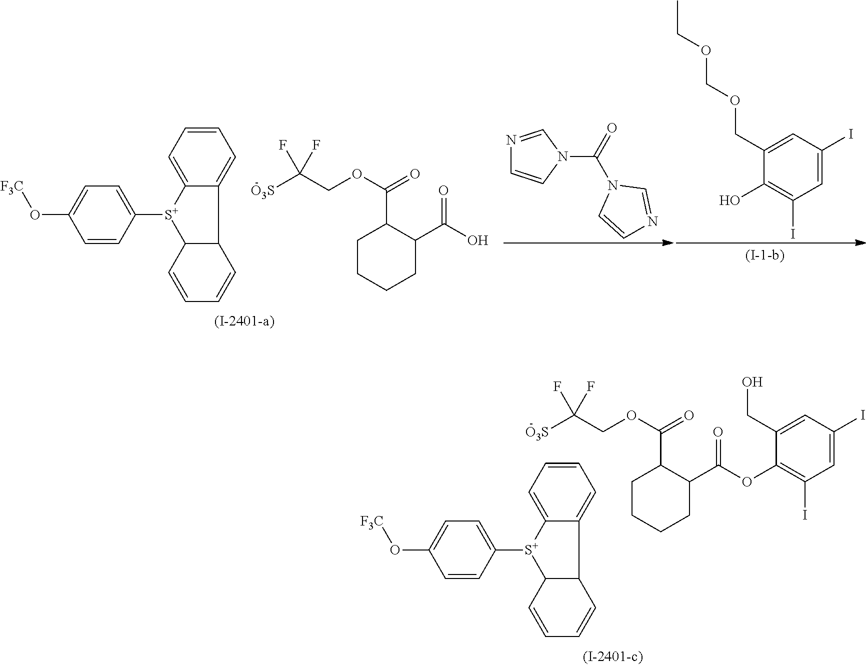

6.63 parts of the salt represented by formula (I-2401-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 9.17 parts of the salt represented by formula (I-2401-c).

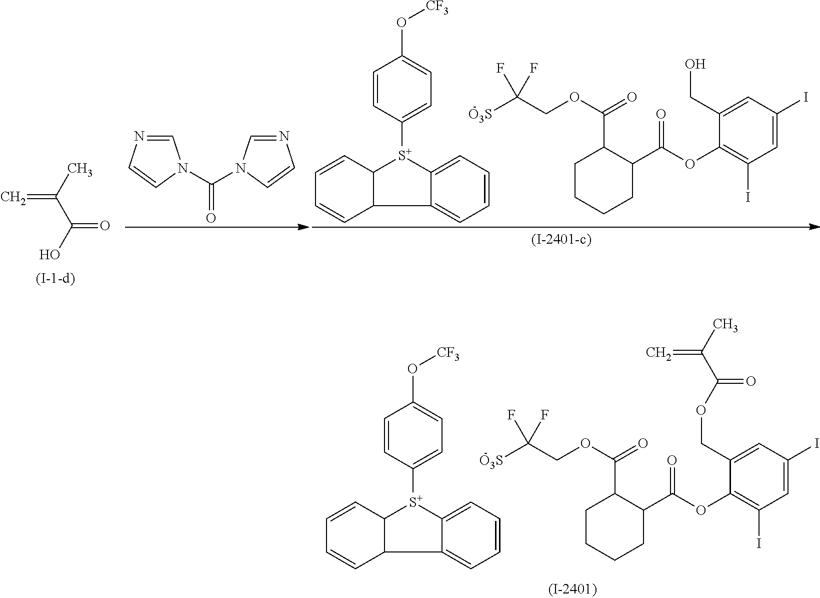

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 5.10 parts of the salt represented by formula (I-2401-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 4.82 parts of the salt represented by formula (I-2401).

MS (ESI (+) Spectrum): M⁺ 347.1

MS (ESI (−) Spectrum): M⁻ 740.9

### Example 6: Synthesis of Salt Represented by Formula (I-5051)

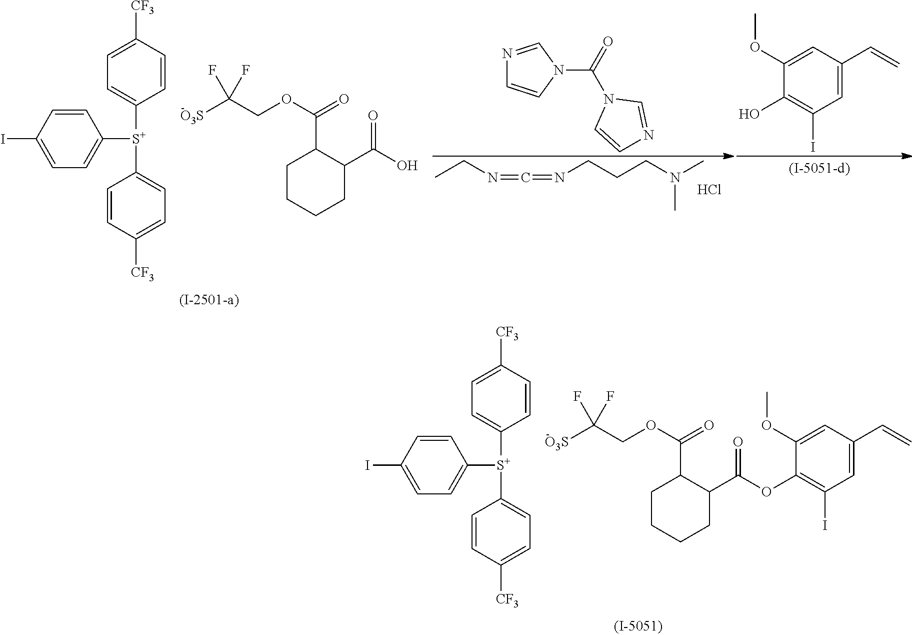

8.41 parts of the salt represented by formula (1-2501-a) and 50 parts of chloroform were mixed and stirred at 23° C. for 30 minutes. To the obtained mixed solution, 1.62 parts of carbonyl diimidazole and 1.92 parts of 1-[3-(dimethylamino)propyl]-3-ethylcarbodiimide hydrochloride were added and stirred for 2 hours at 50° C. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 9.92 parts of the salt represented by formula (I-5051).

MS (ESI (+) Spectrum): M⁺ 525.0

MS (ESI (−) Spectrum): M⁻ 573.0

### Example 7: Synthesis of Salt Represented by Formula (I-5088)

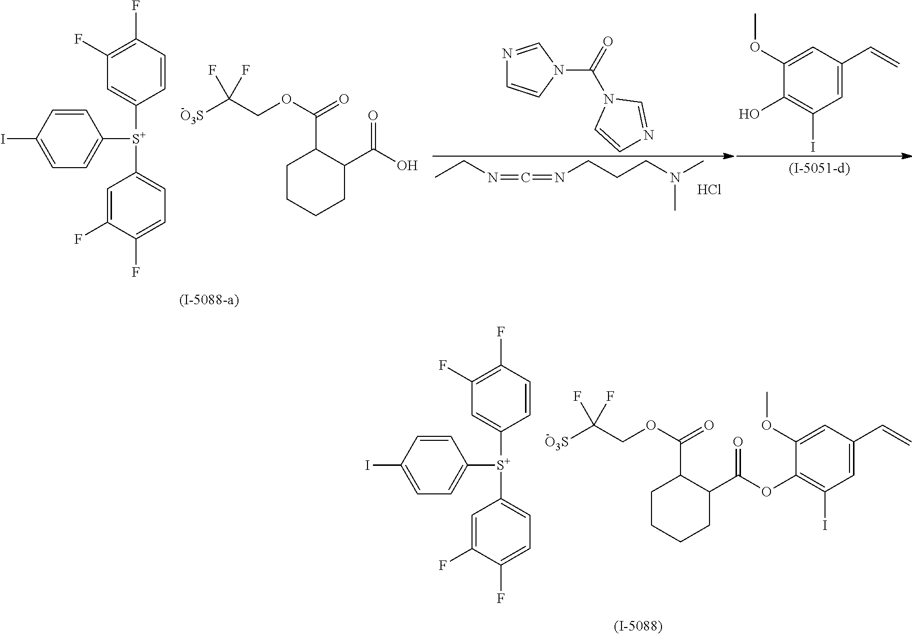

7.77 parts of the salt represented by formula (I-5088-a) and 50 parts of chloroform were mixed and stirred at 23° C. for 30 minutes. To the obtained mixed solution, 1.62 parts of carbonyl diimidazole and 1.92 parts of 1-[3-(dimethylamino)propyl]-3-ethylcarbodiimide hydrochloride were added and stirred for 2 hours at 50° C. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 9.33 parts of the salt represented by formula (I-5088).

MS (ESI (+) Spectrum): M⁺ 461.0

MS (ESI (−) Spectrum): M⁻ 573.0

### Example 8: Synthesis of Salt Represented by Formula (I-5089)

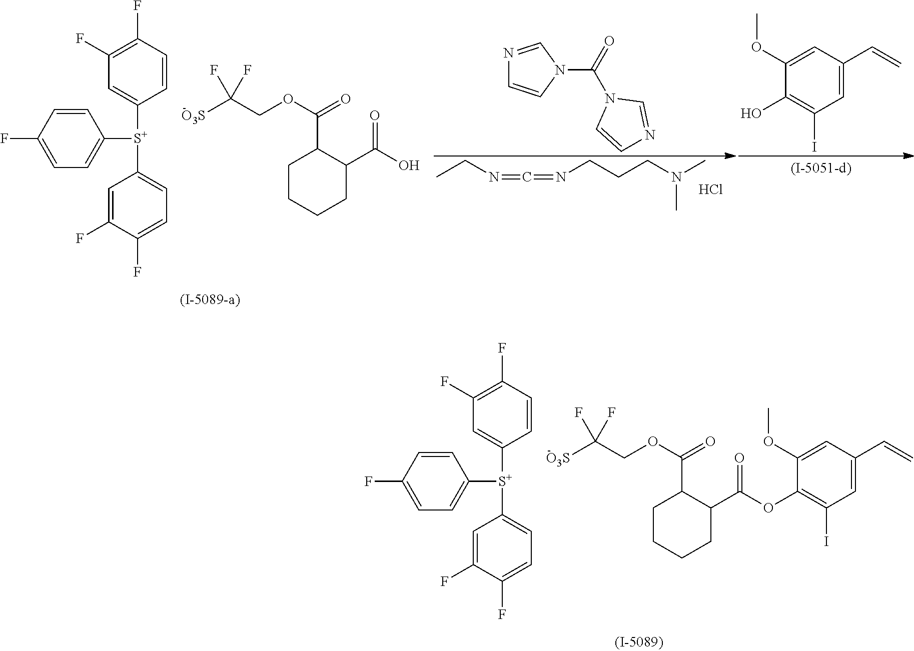

6.69 parts of the salt represented by formula (I-5089-a) and 50 parts of chloroform were mixed and stirred at 23° C. for 30 minutes. To the obtained mixed solution, 1.62 parts of carbonyl diimidazole and 1.92 parts of 1-[3-(dimethylamino)propyl]-3-ethylcarbodiimide hydrochloride were added and stirred for 2 hours at 50° C. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.21 parts of the salt represented by formula (I-5089).

MS (ESI (+) Spectrum): M⁺ 353.0

MS (ESI (−) Spectrum): M⁻ 573.0

### Example 9: Synthesis of Salt Represented by Formula (I-5090)

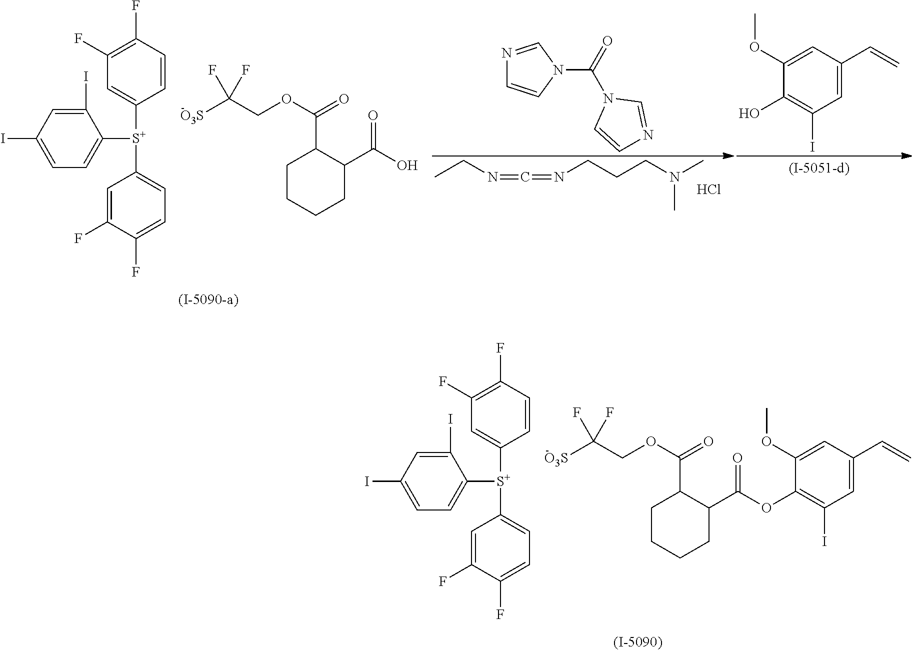

9.02 parts of the salt represented by formula (I-5090-a) and 50 parts of chloroform were mixed and stirred at 23° C. for 30 minutes. To the obtained mixed solution, 1.62 parts of carbonyl diimidazole and 1.92 parts of 1-[3-(dimethylamino)propyl]-3-ethylcarbodiimide hydrochloride were added and stirred for 2 hours at 50° C. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.55 parts of the salt represented by formula (I-5090).

MS (ESI (+) Spectrum): M⁺ 586.8

MS (ESI (−) Spectrum): M⁻ 573.0

### Example 10: Synthesis of Salt Represented by Formula (I-2548)

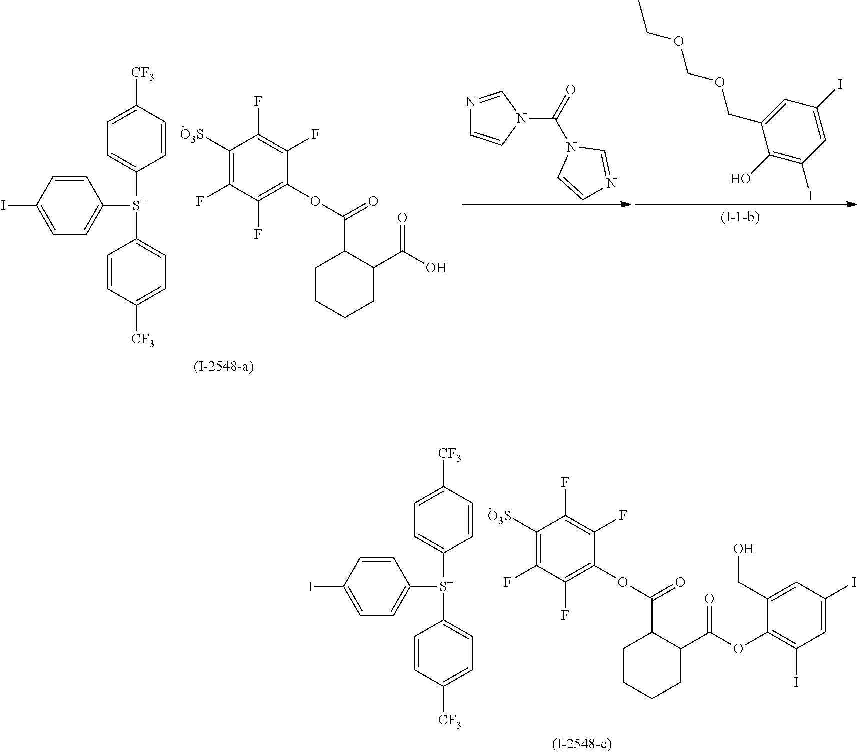

9.25 parts of the salt represented by formula (I-2548-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 11.39 parts of the salt represented by formula (I-2548-c).

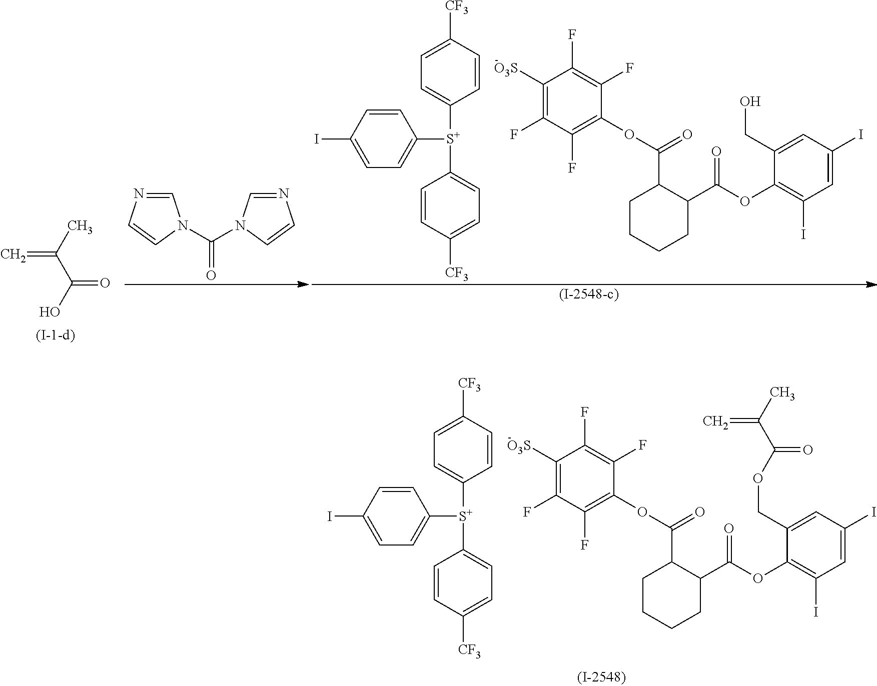

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 6.41 parts of the salt represented by formula (I-2548-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 6.23 parts of the salt represented by formula (I-2548).

MS (ESI (+) Spectrum): M⁺ 525.0

MS (ESI (−) Spectrum): M⁻ 824.9

### Example 11: Synthesis of Salt Represented by Formula (I-5951)

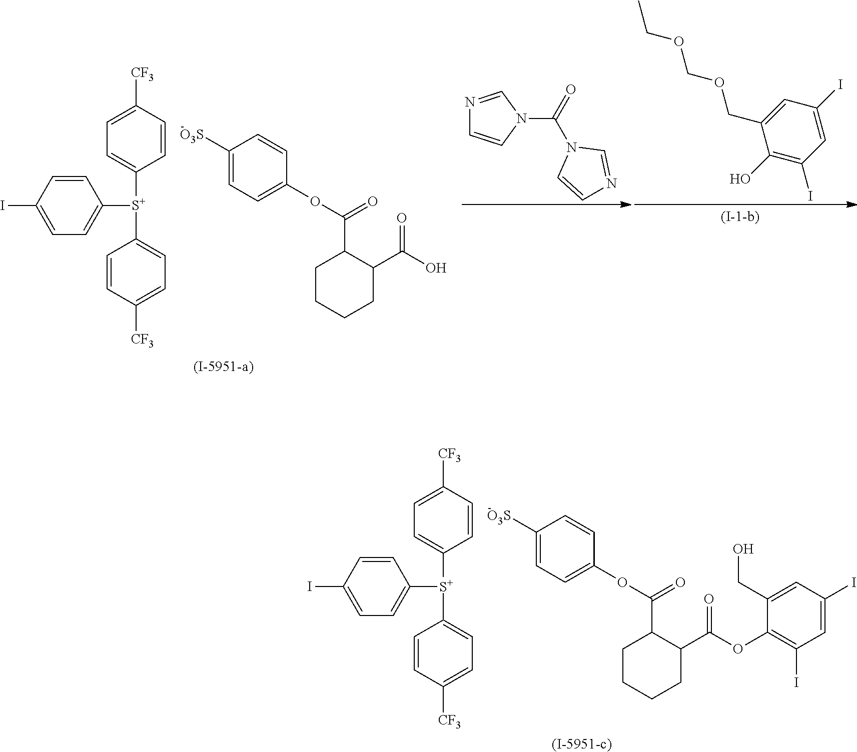

8.53 parts of the salt represented by formula (I-5951-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.82 parts of the salt represented by formula (I-5951-c).

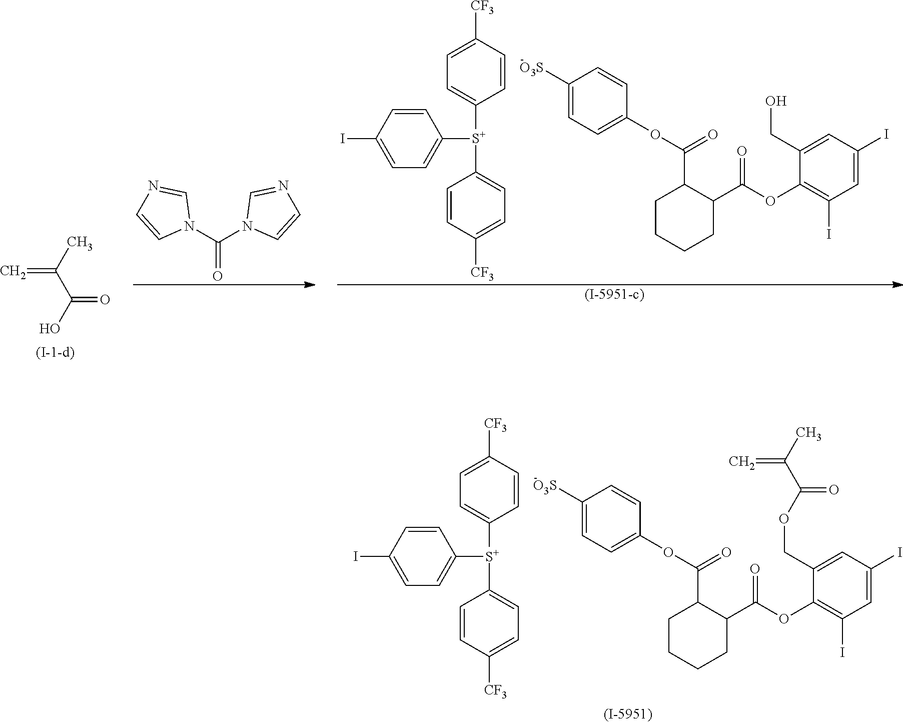

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 6.05 parts of the salt represented by formula (I-5951-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 5.77 parts of the salt represented by formula (I-5951).

MS (ESI (+) Spectrum): M⁺ 525.0

MS (ESI (−) Spectrum): M⁻ 752.9

### Example 12: Synthesis of Salt Represented by Formula (I-6051)

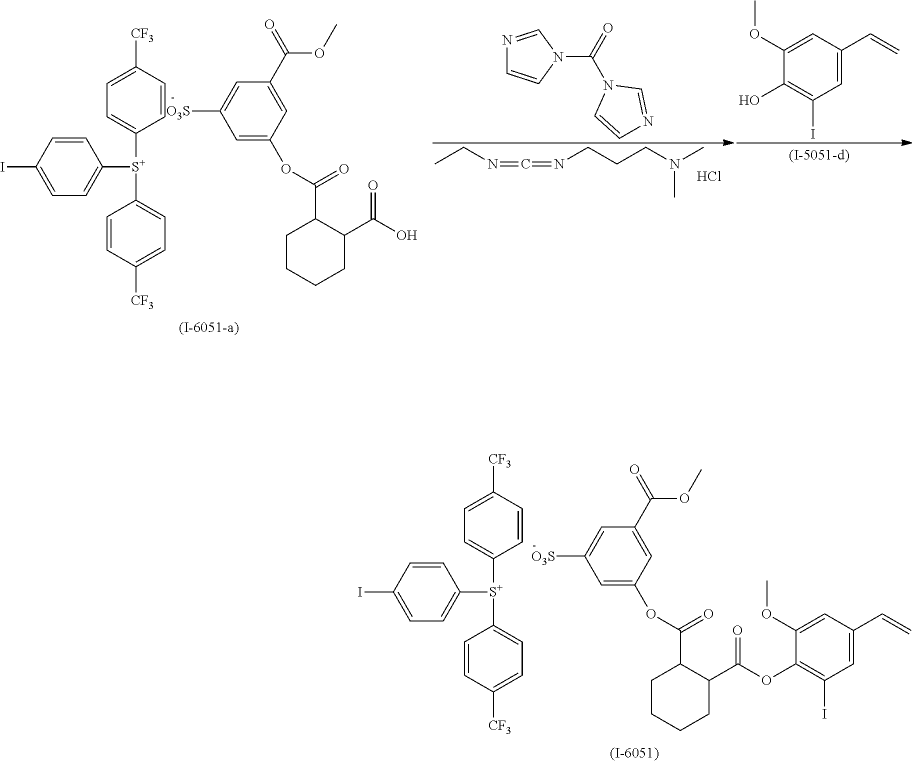

9.11 parts of the salt represented by formula (I-6051-a) and 50 parts of chloroform were mixed and stirred at 23° C. for 30 minutes. To the obtained mixed solution, 1.62 parts of carbonyl diimidazole and 1.92 parts of 1-[3-(dimethylamino)propyl]-3-ethylcarbodiimide hydrochloride were added and stirred for 2 hours at 50° C. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.18 parts of the salt represented by formula (I-6051).

MS (ESI (+) Spectrum): M⁺ 525.0

MS (ESI (−) Spectrum): M⁻ 643.0

### Example 13: Synthesis of Salt Represented by Formula (I-6351)

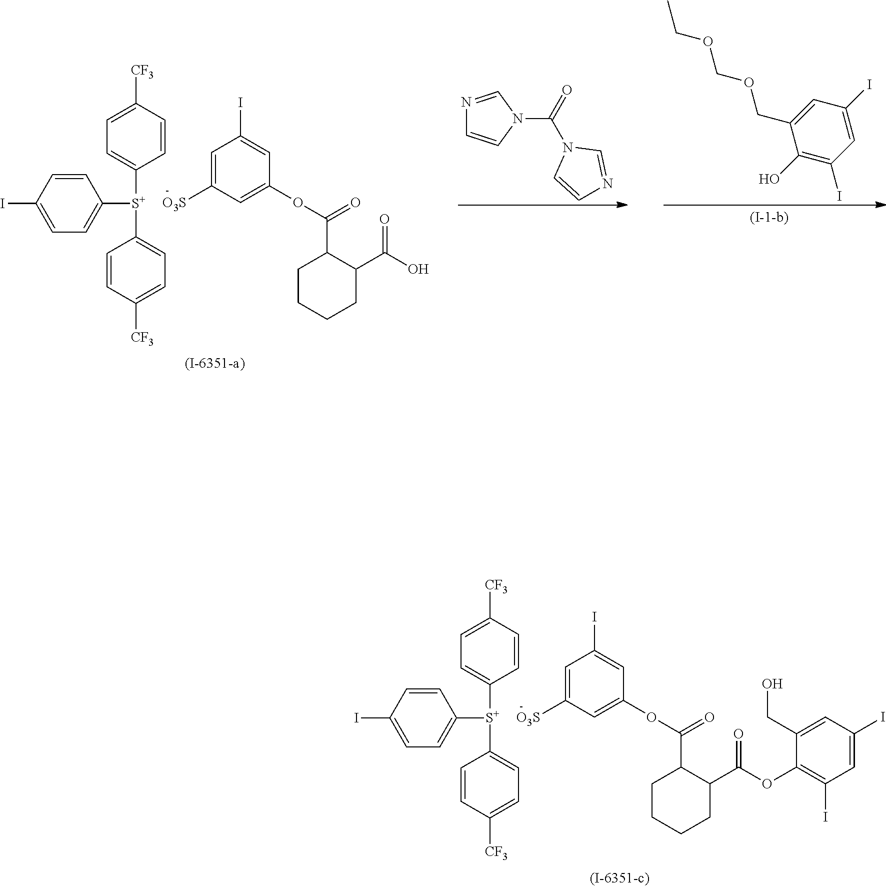

9.78 parts of the salt represented by formula (I-6351-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 11.84 parts of the salt represented by formula (I-6351-c).

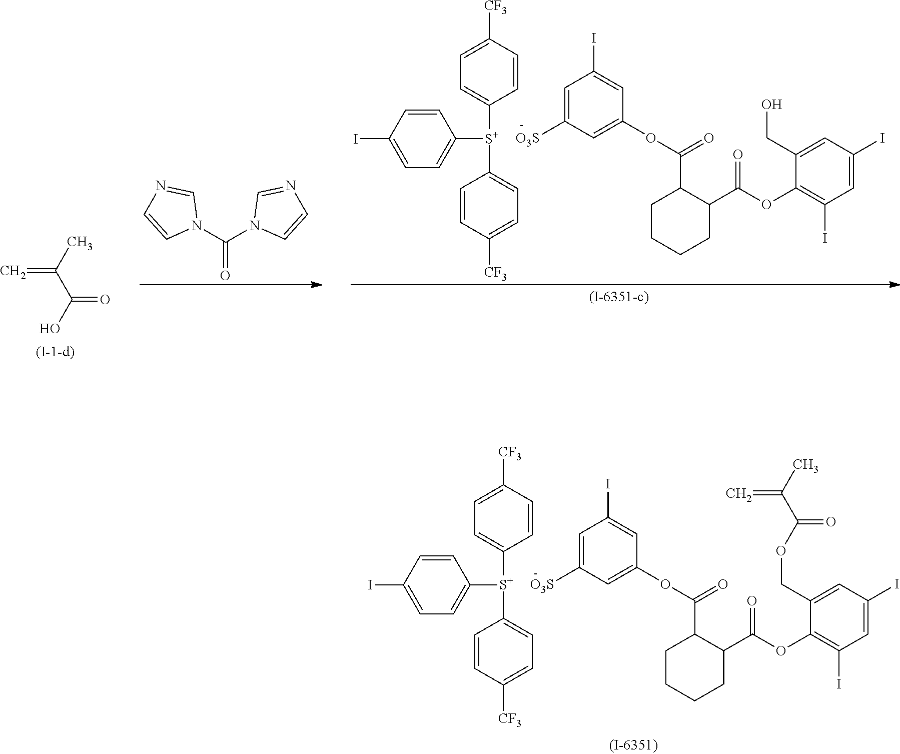

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 6.68 parts of the salt represented by formula (I-6351-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 5.97 parts of the salt represented by formula (I-6351).

MS (ESI (+) Spectrum): M⁺ 525.0

MS (ESI (−) Spectrum): M⁻ 878.8

### Example 14: Synthesis of Salt Represented by Formula (I-5451)

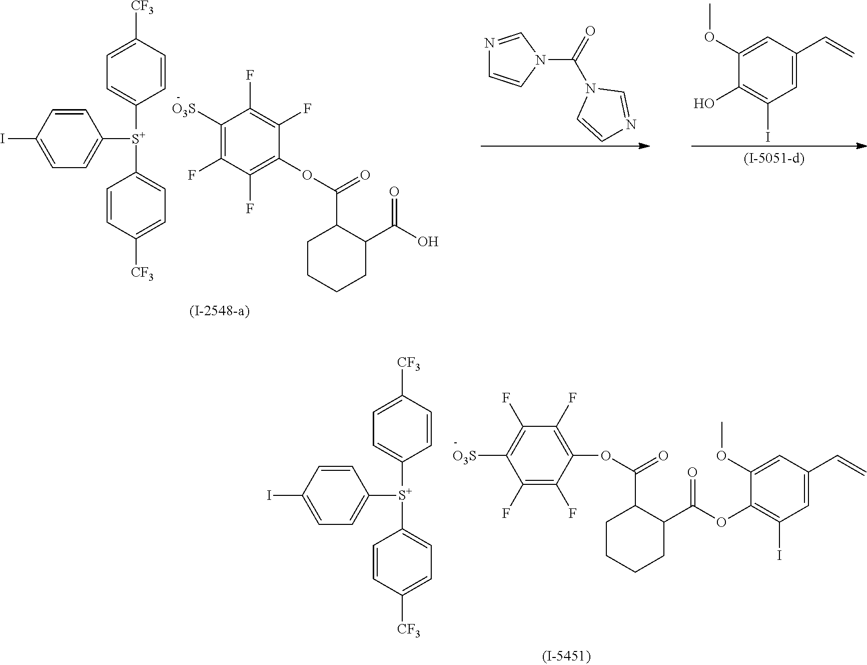

9.25 parts of the salt represented by formula (I-2548-a) and 50 parts of chloroform were mixed and stirred at 23° C. for 30 minutes. 1.62 parts of carbonyldiimidazole was added to the resulting mixed solution and stirred at 50° C. for 2 hours. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.14 parts of the salt represented by formula (I-5451).

MS (ESI (+) Spectrum): M⁺ 525.0

MS (ESI (−) Spectrum): M⁻ 657.0

### Example 15: Synthesis of Salt Represented by Formula (I-5551)

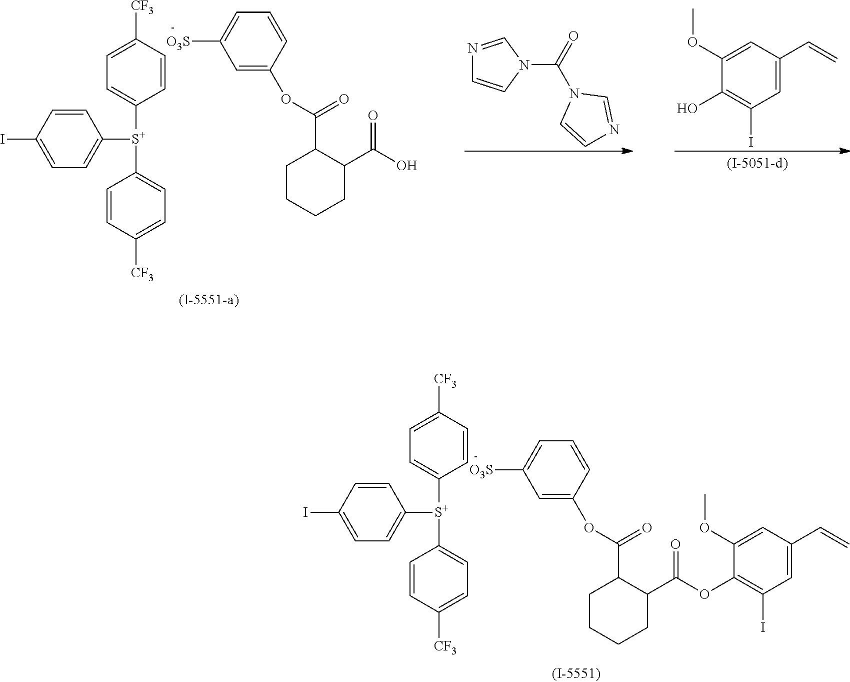

8.53 parts of the salt represented by formula (I-5551-a) and 50 parts of chloroform were mixed and stirred at 23° C. for 30 minutes. 1.62 parts of carbonyldiimidazole was added to the resulting mixed solution and stirred at 50° C. for 2 hours. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.79 parts of the salt represented by formula (I-5551).

MS (ESI (+) Spectrum): M⁺ 525.0

MS (ESI (−) Spectrum): M⁻ 585.0

### Example 16: Synthesis of Salt Represented by Formula (I-5751)

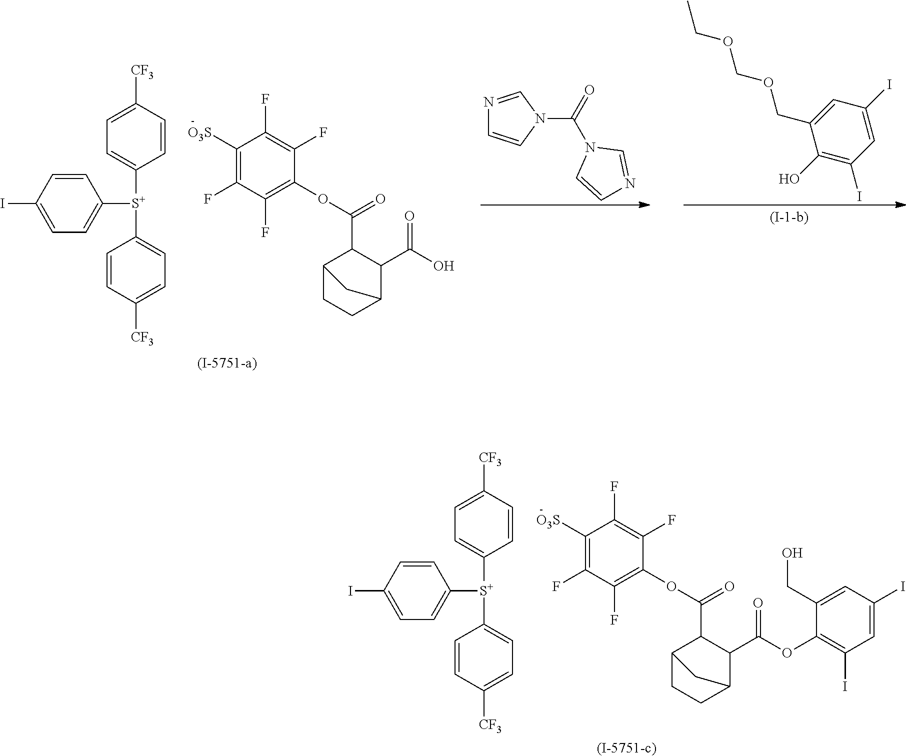

9.37 parts of the salt represented by formula (I-5751-a) and 50 parts of chloroform were mixed and stirred at 23° C. for 30 minutes. 1.62 parts of carbonyldiimidazole was added to the resulting mixed solution and stirred at 50° C. for 2 hours. 4.34 parts of the compound represented by formula (I-1-b) was added to the resulting reaction solution and stirred at 50° C. for 3 hours, and then cooled to 23° C. 25 parts of 1 N hydrochloric acid was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 10.43 parts of the salt represented by formula (I-5751-c).

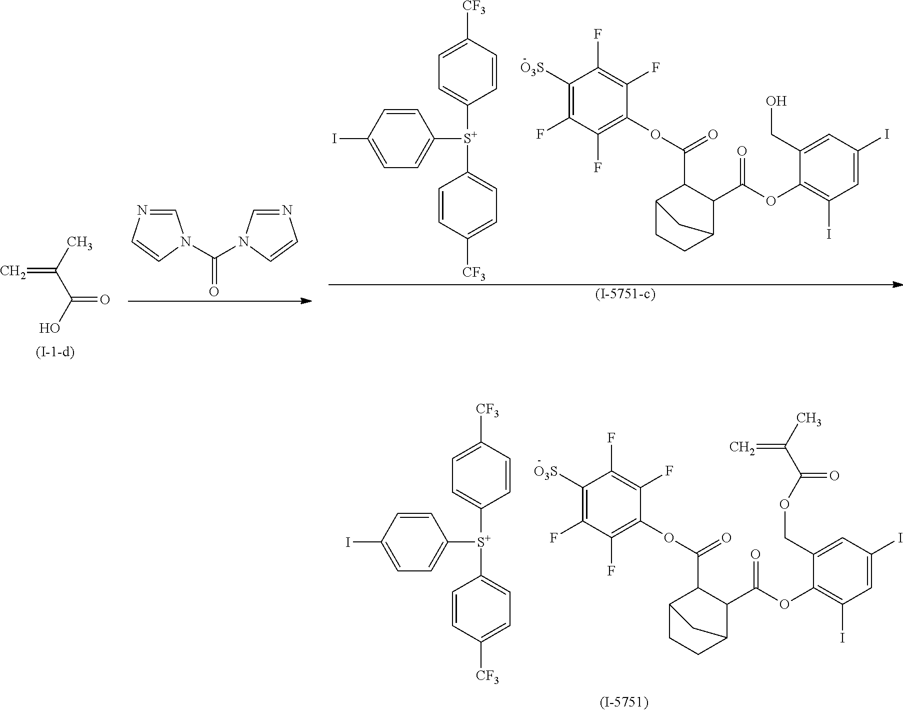

0.43 parts of the compound represented by formula (I-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 6.47 parts of the salt represented by formula (I-5751-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 6.11 parts of the salt represented by formula (I-5751).

MS (ESI (+) Spectrum): M⁺ 525.0

MS (ESI (−) Spectrum): M⁻ 836.9

### Example 17: Synthesis of Salt Represented by Formula (I-5489)

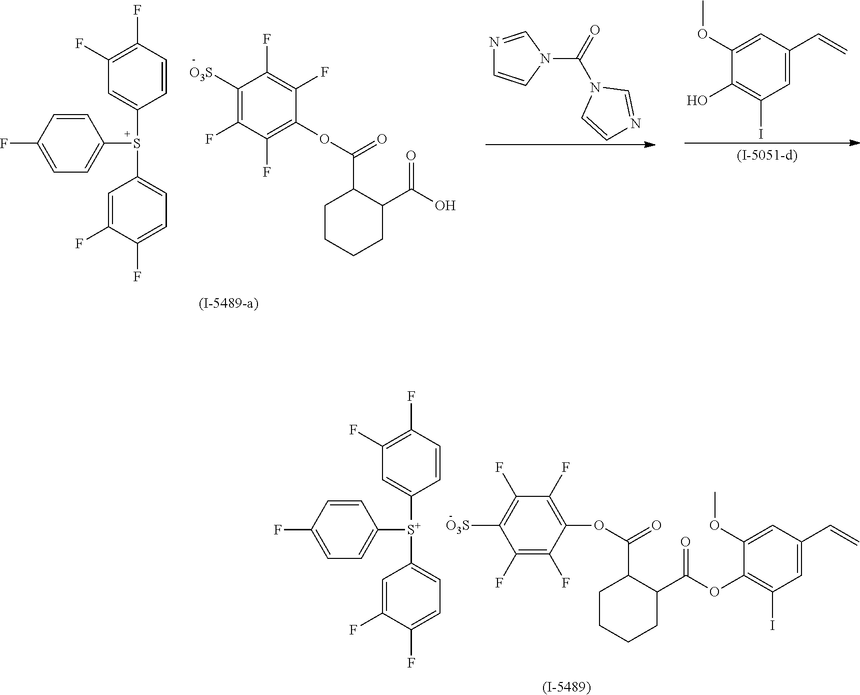

7.53 parts of the salt represented by formula (I-5489-a) and 50 parts of chloroform were mixed and stirred at 23° C. for 30 minutes. 1.62 parts of carbonyldiimidazole was added to the resulting mixed solution and stirred at 50° C. for 2 hours. 2.76 parts of the compound represented by formula (I-5051-d) was added to the resulting reaction solution and stirred at 50° C. for 3 hours, and then cooled to 23° C. 25 parts of a 5% aqueous oxalic acid solution was added to the resulting mixture and stirred at 23° C. for 30 minutes, and then the organic layer was separated and taken out. 25 parts of ion-exchanged water was added to the resulting organic layer and stirred at 23° C. for 30 minutes, and then the organic layer was separated and taken out. This water washing procedure was repeated three times. The resulting organic layer was concentrated, and 30 parts of tert-butyl methyl ether was added to the concentrated residue, and then stirred at 23° C. for 30 minutes. The supernatant was then removed, and the mixture was concentrated to provide 8.22 parts of the salt represented by formula (I-5489).

MS (ESI (+) Spectrum): M⁺ 353.0

MS (ESI (−) Spectrum): M⁻ 657.0

### Synthesis Example 1: Synthesis of Salt Represented by Formula (IX-1)

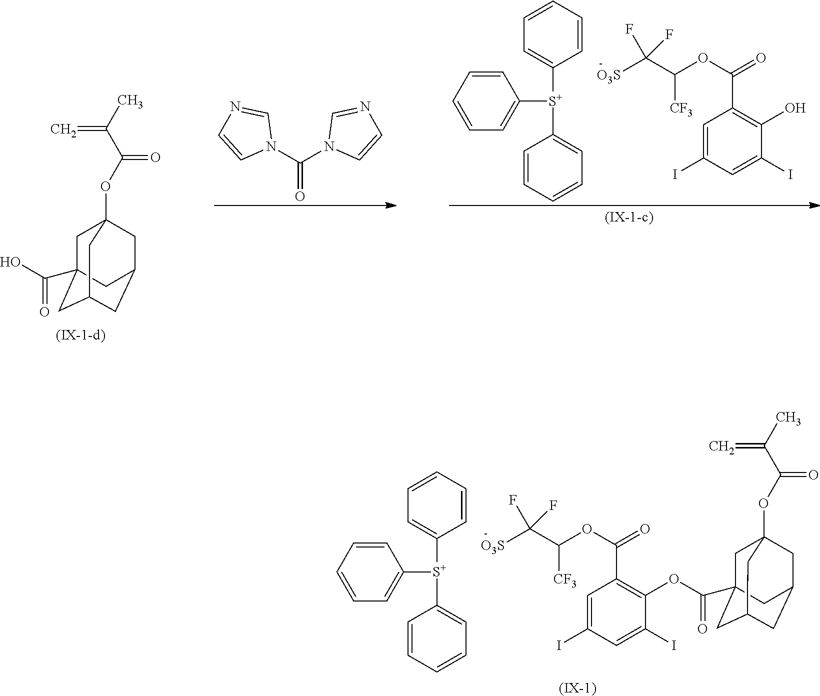

1.32 parts of the compound represented by formula (IX-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 4.32 parts of the salt represented by formula (IX-1-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 4.16 parts of the salt represented by formula (IX-1).

MS (ESI (+) Spectrum): M⁺ 263.1

MS (ESI (−) Spectrum): M⁻ 846.9

### Synthesis Example 2: Synthesis of Salt Represented by Formula (IX-2)

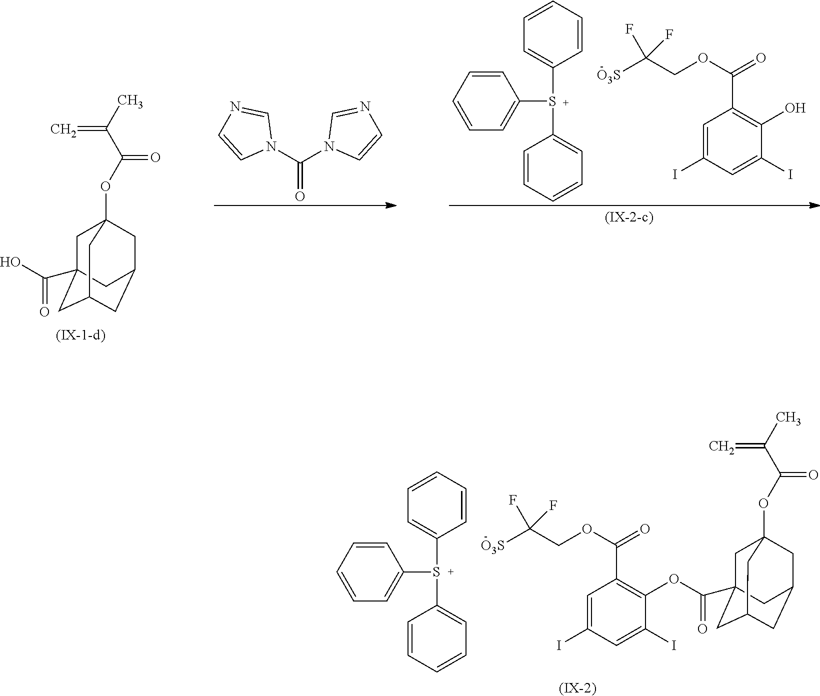

1.32 parts of the compound represented by formula (IX-1-d) and 20 parts of acetonitrile were mixed and stirred for 30 minutes at 23° C. Then, 0.81 parts of carbonyl diimidazole was added thereto, and the temperature was increased to 50° C., followed by stirring for 2 hours at 50° C. To the obtained mixture, 3.98 parts of the salt represented by formula (IX-2-c) was added and stirred for 2 hours at 50° C. After the obtained mixture was cooled to 23° C., 50 parts of chloroform and 20 parts of ion-exchanged water were added and stirred for 30 minutes at 23° C., and then the organic layer was separated and taken out. 20 parts of ion-exchanged water was added to the resulting organic layer and stirred at 23° C. for 30 minutes, and then the organic layer was separated and taken out. This water washing procedure was repeated five times. The resulting organic layer was concentrated, and 3 parts of acetonitrile and 30 parts of tert-butyl methyl ether were added to the concentrated residue, and then stirred at 23° C. for 30 minutes. The supernatant was then removed, and the mixture was concentrated to provide 4.33 parts of the salt represented by formula (IX-2).

MS (ESI (+) Spectrum): M⁺ 263.1

MS (ESI (−) Spectrum): M⁻ 778.9

### Synthesis of Resin

The compounds (monomers) used in the synthesis of the resins are shown below.

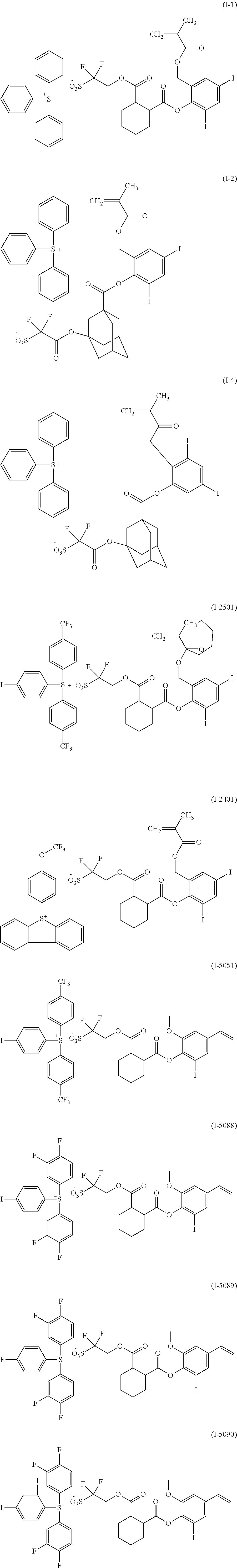

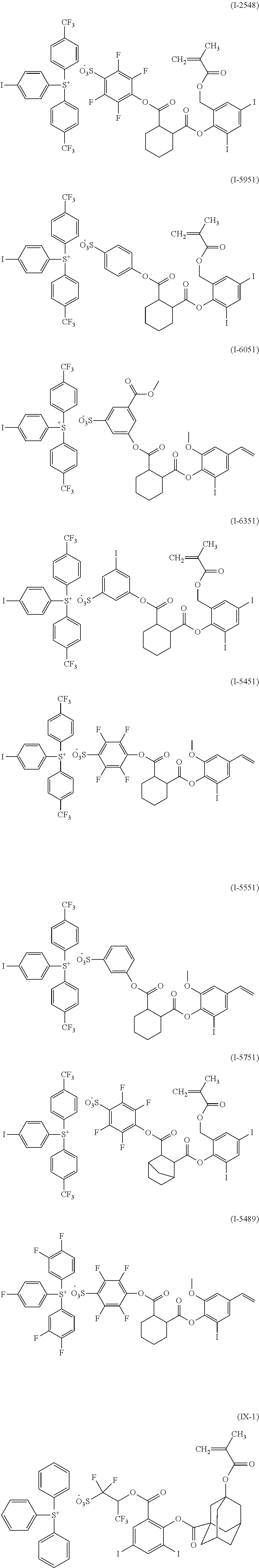

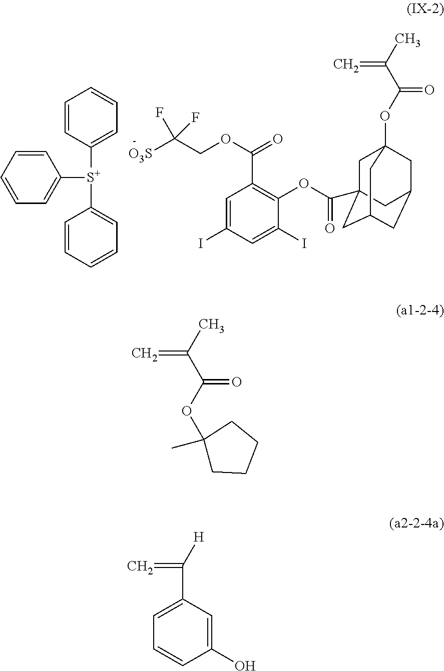

Hereinafter, these compounds will be referred to as “monomers (a2-2-1)” and the like according to the formula numbers thereof.

### Example 18 [Synthesis of Resin A1]

As monomers, monomer (a2-2-4a), monomer (a1-2-4), and monomer (I-1) were used and mixed so that the molar ratio thereof [monomer (a2-2-4a): monomer (a1-2-4): monomer (I-1)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A1 (copolymer) having a weight average molecular weight of about 5.6×10³ in a yield of 88%. This resin A1 has the following structural unit.

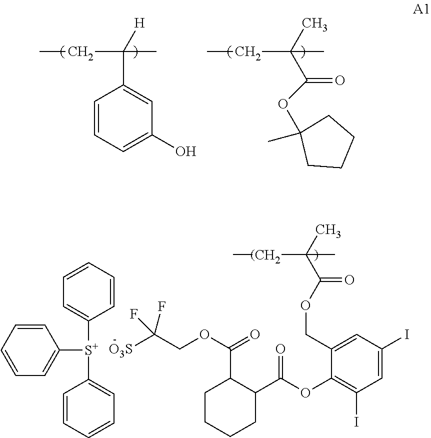

### Example 19 [Synthesis of Resin A2]

As monomers, monomer (a2-2-4a), monomer (a1-2-4), and monomer (I-2) were used and mixed so that the molar ratio thereof [monomer (a2-2-4a): monomer (a1-2-4): monomer (I-2)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A2 (copolymer) having a weight average molecular weight of about 5.5×10³ in a yield of 83%. This resin A2 has the following structural unit.

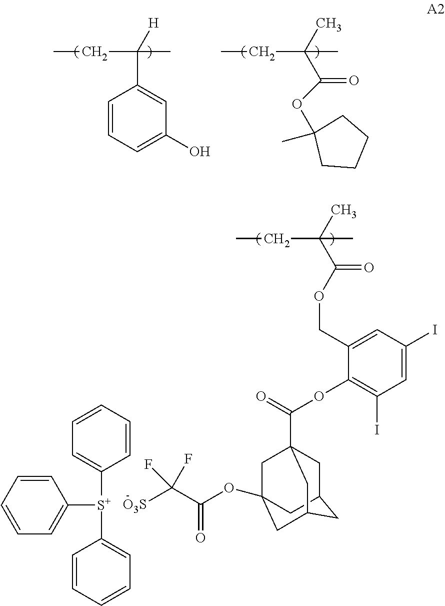

### Example 20 [Synthesis of Resin A3]

As monomers, monomer (a2-2-4a), monomer (a1-2-4), and monomer (I-4) were used and mixed so that the molar ratio thereof [monomer (a2-2-4a):monomer (a1-2-4):monomer (I-4)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A3 (copolymer) having a weight average molecular weight of about 5.3×10³ in a yield of 77%. This resin A3 has the following structural unit.

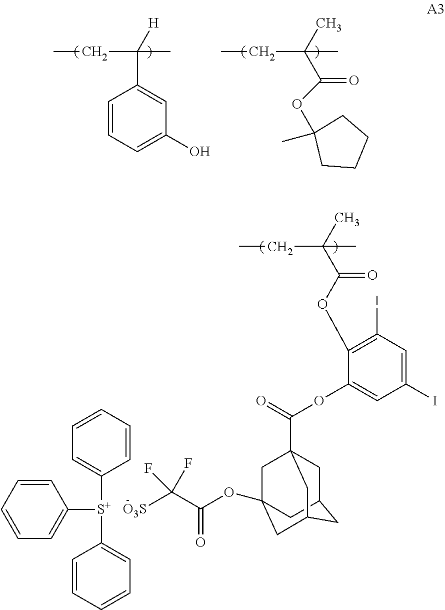

### Example 21 [Synthesis of Resin A4]

As monomers, monomer (a2-2-4a), monomer (a1-2-4), and monomer (I-2501) were used and mixed so that the molar ratio thereof [monomer (a2-2-4a): monomer (a1-2-4): monomer (I-2501)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A4 (copolymer) having a weight average molecular weight of about 5.4×10³ in a yield of 83%. This resin A4 has the following structural unit.

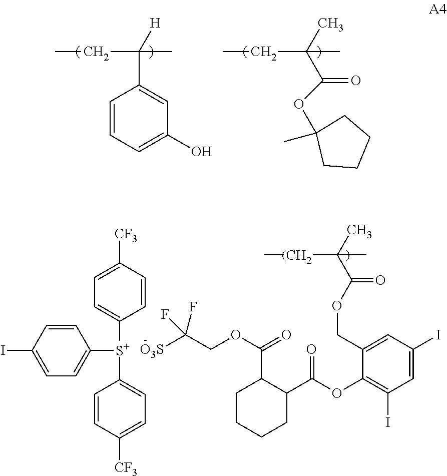

### Example 22 [Synthesis of Resin A5]

As monomers, monomer (a2-2-4a), monomer (a1-2-4), and monomer (I-2401) were used and mixed so that the molar ratio thereof [monomer (a2-2-4a): monomer (a1-2-4): monomer (I-2401)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A5 (copolymer) having a weight average molecular weight of about 5.4×10³ in a yield of 79%. This resin A5 has the following structural unit.

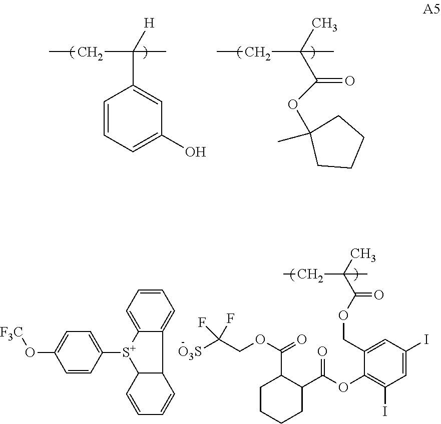

### Example 23 [Synthesis of Resin A6]

As monomers, monomer (a2-2-4a), monomer (a1-2-4), and monomer (I-5051) were used and mixed so that the molar ratio thereof [monomer (a2-2-4a): monomer (a1-2-4): monomer (I-5051)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A6 (copolymer) having a weight average molecular weight of about 5.3×10³ in a yield of 84%. This resin A6 has the following structural unit.

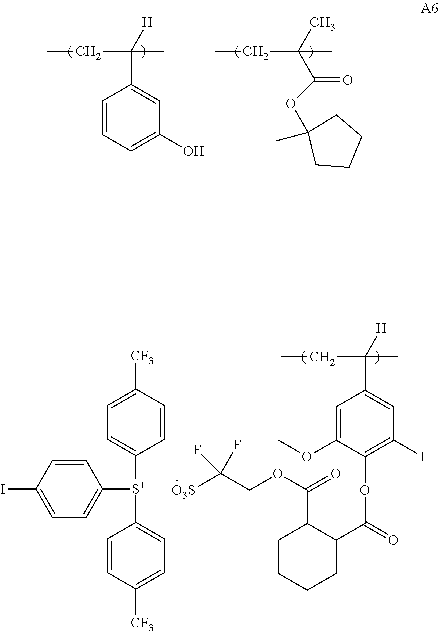

### Example 24 [Synthesis of Resin A7]

As monomers, monomer (a2-2-4a), monomer (a1-2-4), and monomer (I-5088) were used and mixed so that the molar ratio thereof [monomer (a2-2-4a): monomer (a1-2-4): monomer (I-5088)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A7 (copolymer) having a weight average molecular weight of about 5.4×10³ in a yield of 85%. This resin A7 has the following structural unit.

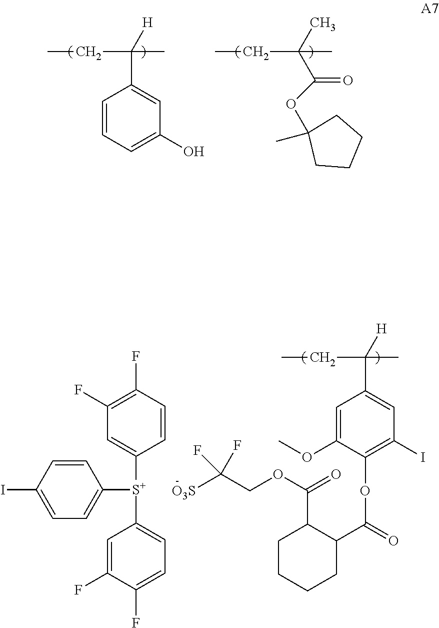

### Example 25 [Synthesis of Resin A8]

As monomers, monomer (a2-2-4a), monomer (a1-2-4), and monomer (I-5089) were used and mixed so that the molar ratio thereof [monomer (a2-2-4a): monomer (a1-2-4): monomer (I-5089)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A8 (copolymer) having a weight average molecular weight of about 5.6×10³ in a yield of 86%. This resin A8 has the following structural unit.

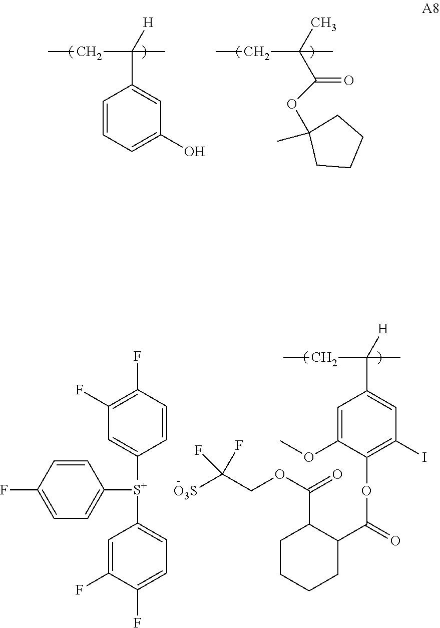

### Example 26 [Synthesis of Resin A9]

As monomers, monomer (a2-2-4a), monomer (a1-2-4), and monomer (I-5090) were used and mixed so that the molar ratio thereof [monomer (a2-2-4a): monomer (a1-2-4): monomer (I-5090)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A9 (copolymer) having a weight average molecular weight of about 5.2×10³ in a yield of 79%. This resin A9 has the following structural unit.

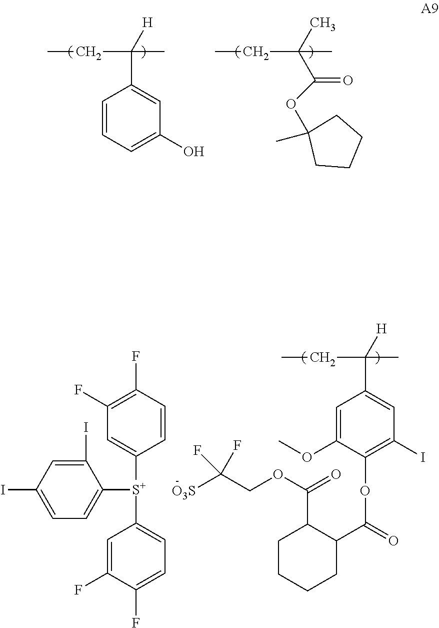

### Example 27 [Synthesis of Resin A10]

As monomers, monomer (a2-2-4a), monomer (a1-2-4), and monomer (I-2548) were used and mixed so that the molar ratio thereof [monomer (a2-2-4a): monomer (a1-2-4): monomer (I-2548)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A10 (copolymer) having a weight average molecular weight of about 5.3×10³ in a yield of 75%. This resin A10 has the following structural unit.

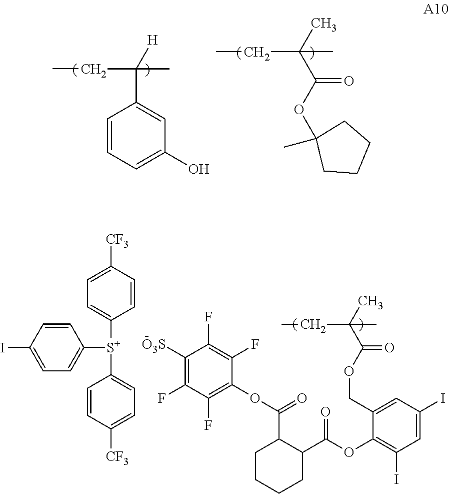

### Example 28 [Synthesis of Resin A11]

As monomers, monomer (a2-2-4a), monomer (a1-2-4), and monomer (I-5951) were used and mixed so that the molar ratio thereof [monomer (a2-2-4a): monomer (a1-2-4): monomer (I-5951)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A11 (copolymer) having a weight average molecular weight of about 5.6×10³ in a yield of 84%. This resin A11 has the following structural unit.

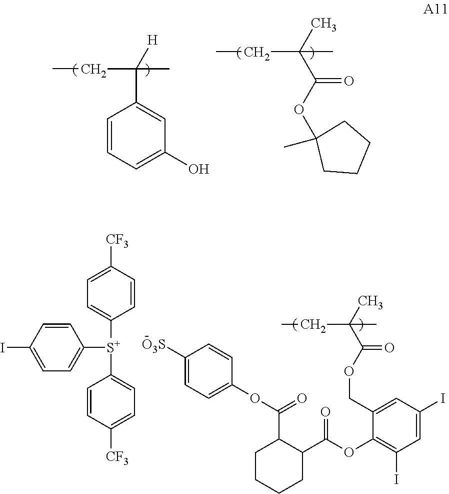

### Example 29 [Synthesis of Resin A12]

As monomers, monomer (a2-2-4a), monomer (a1-2-4), and monomer (I-6051) were used and mixed so that the molar ratio thereof [monomer (a2-2-4a): monomer (a1-2-4): monomer (I-6051)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A12 (copolymer) having a weight average molecular weight of about 5.6×10³ in a yield of 84%. This resin A12 has the following structural unit.

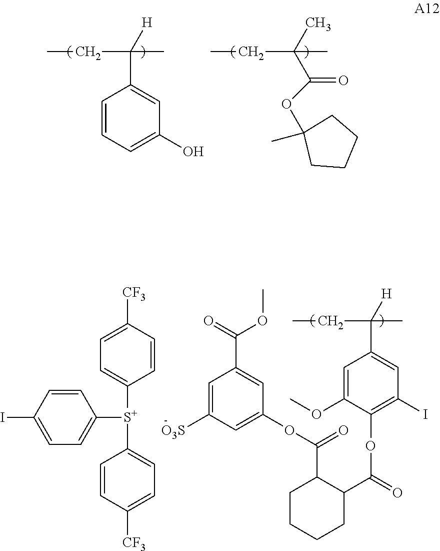

### Example 30 [Synthesis of Resin A13]

As monomers, monomer (a2-2-4a), monomer (a1-2-4), and monomer (I-6351) were used and mixed so that the molar ratio thereof [monomer (a2-2-4a): monomer (a1-2-4): monomer (I-6351)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A13 (copolymer) having a weight average molecular weight of about 5.2×10³ in a yield of 76%. This resin A13 has the following structural unit.

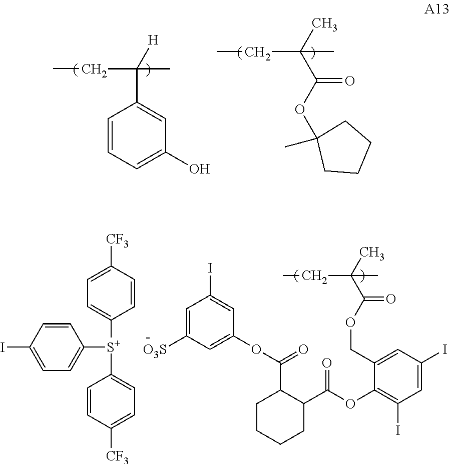

### Example 31 [Synthesis of Resin A14]

As monomers, monomer (a2-2-4a), monomer (a1-2-4), and monomer (I-5451) were used and mixed so that the molar ratio thereof [monomer (a2-2-4a): monomer (a1-2-4): monomer (I-5451)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A14 (copolymer) having a weight average molecular weight of about 5.4×10³ in a yield of 82%. This resin A14 has the following structural unit.

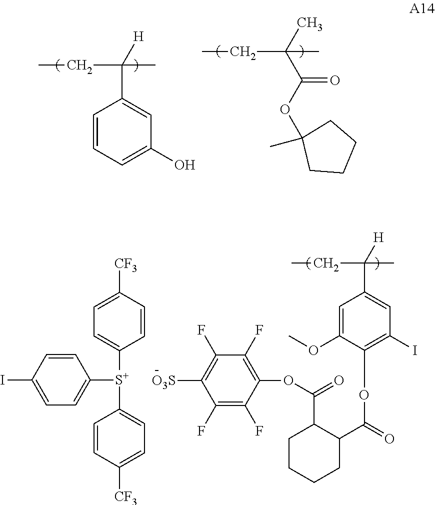

### Example 32 [Synthesis of Resin A15]

As monomers, monomer (a2-2-4a), monomer (a1-2-4), and monomer (I-5551) were used and mixed so that the molar ratio thereof [monomer (a2-2-4a): monomer (a1-2-4): monomer (I-5551)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A15 (copolymer) having a weight average molecular weight of about 5.6×10³ in a yield of 85%. This resin A15 has the following structural unit.

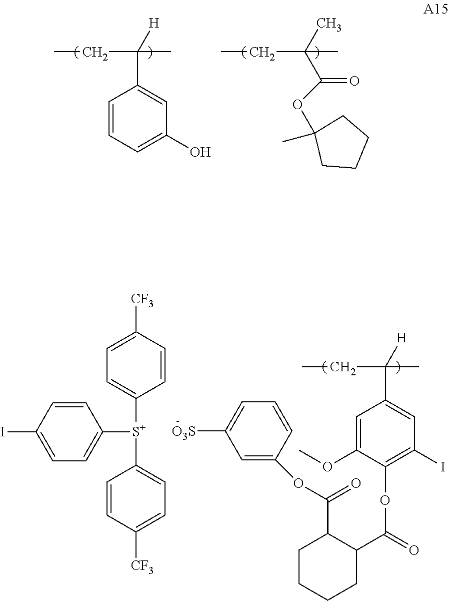

### Example 33 [Synthesis of Resin A16]

As monomers, monomer (a2-2-4a), monomer (a1-2-4), and monomer (I-5751) were used and mixed so that the molar ratio thereof [monomer (a2-2-4a): monomer (a1-2-4): monomer (I-5751)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A16 (copolymer) having a weight average molecular weight of about 5.4×10³ in a yield of 71%. This resin A16 has the following structural unit.

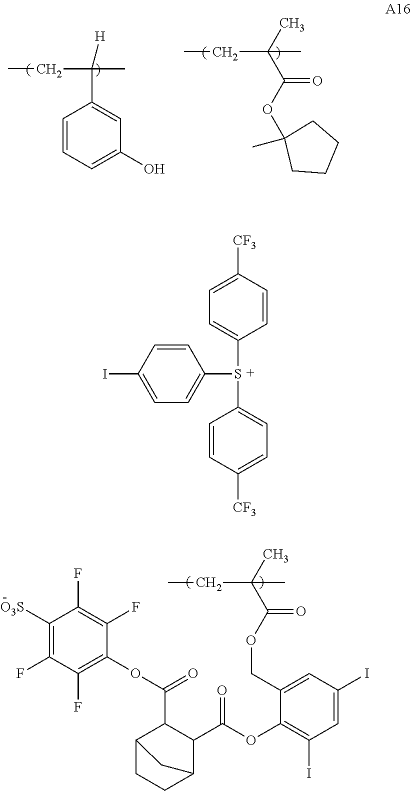

### Example 34 [Synthesis of Resin A17]

As monomers, monomer (a2-2-4a), monomer (a1-2-4), and monomer (I-5489) were used and mixed so that the molar ratio thereof [monomer (a2-2-4a): monomer (a1-2-4): monomer (I-5489)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin A17 (copolymer) having a weight average molecular weight of about 5.6×10³ in a yield of 85%. This resin A17 has the following structural unit.

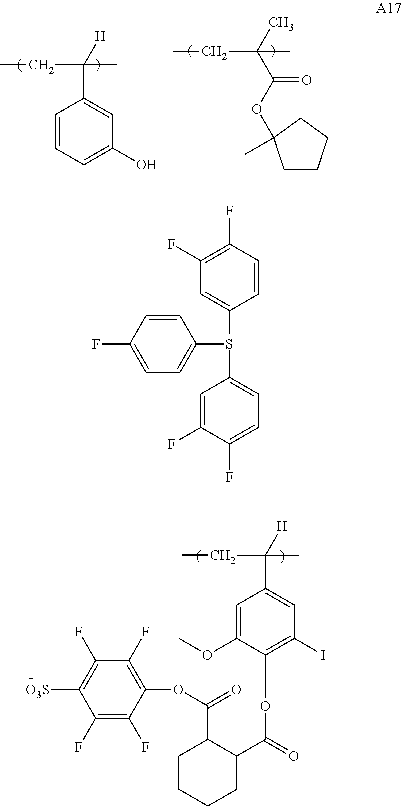

### Synthesis Example 3 [Synthesis of Resin AX1]

As monomers, monomer (a2-2-4a), monomer (a1-2-4), and monomer (IX-1) were used and mixed so that the molar ratio thereof [monomer (a2-2-4a): monomer (a1-2-4): monomer (IX-1)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin AX1 (copolymer) having a weight average molecular weight of about 5.2×10³ in a yield of 73%. This resin AX1 has the following structural unit.

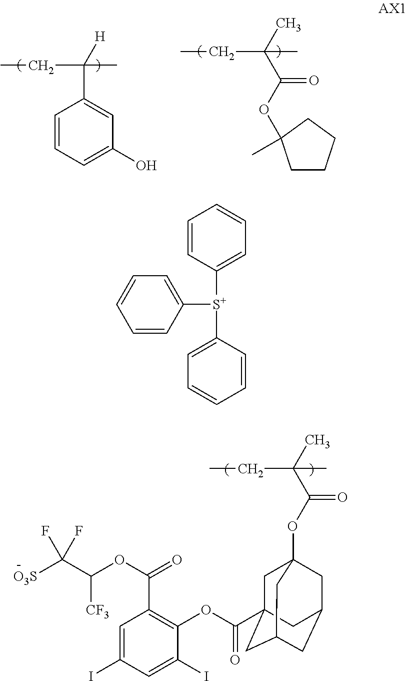

### Synthesis Example 4 [Synthesis of Resin AX2]

As monomers, monomer (a2-2-4a), monomer (a1-2-4), and monomer (IX-2) were used and mixed so that the molar ratio thereof [monomer (a2-2-4a): monomer (a1-2-4): monomer (IX-2)] was a proportion of 38:47:15. Furthermore, propylene glycol monomethyl ether was mixed into this monomer mixture in an amount of 1.5 times the total mass of all monomers. Azobisisobutyronitrile was added as an initiator to the resulting mixture in an amount of 7 mol % with respect to the total mole number of all monomers, and polymerization was performed by heating at 85° C. for about 5 hours. Then, the polymerization reaction liquid was poured into a large amount of n-heptane to precipitate the resin, and the resin was filtered and collected to provide resin AX2 (copolymer) having a weight average molecular weight of about 5.5×10³ in a yield of 860. This resin AX2 has the following structural unit.

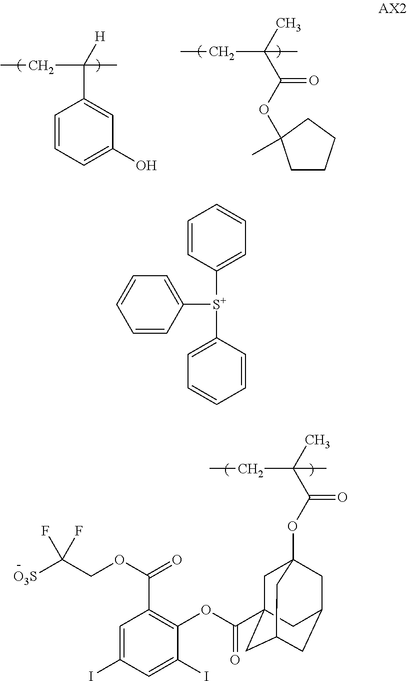

### <Preparation of Resist Composition>

As shown in Table 2, the respective components and the following solvents were mixed and the resulting mixture was filtered through a fluororesin filter with a pore size of 0.2 μm to prepare a resist composition.

TABLE 2

| | | Acid | | | |
|---|---|---|---|---|---|
| Resist | | generator | | | |
| composition | Resin (A) | (B) | Salt (I) | Quencher (C) | PB/PEB |
| Composition | A1 = 10 parts | — | — | C1-1 = 0.35 | 100° C./130° C. |
| 1 | | | | parts | |
| Composition | A2 = 10 parts | — | — | C1-1 = 0.35 | 100° C./130° C. |
| 2 | | | | parts | |
| Composition | A3 = 10 parts | — | — | C1-1 = 0.35 | 100° C./130° C. |
| 3 | | | | parts | |
| Composition | A4 = 10 parts | — | — | C1-1 = 0.35 | 100° C./130° C. |
| 4 | | | | parts | |
| Composition | A5 = 10 parts | — | — | C1-1 = 0.35 | 100° C./130° C. |
| 5 | | | | parts | |
| Composition | A5 = 10 parts | — | I-2401 = | C1-1 = 0.35 | 100° C./130° C. |
| 6 | | | 0.3 parts | parts | |
| Composition | A5 = 10 parts | B1-25 = 0.3 | — | C1-1 = 0.35 | 100° C./130° C. |
| 7 | | parts | | parts | |
| Composition | A6 = 10 parts | — | — | C1-1 = 0.35 | 100° C./130° C. |
| 8 | | | | parts | |
| Composition | A7 = 10 parts | — | — | C1-1 = 0.35 | 100° C./130° C. |
| 9 | | | | parts | |
| Composition | A8 = 10 parts | — | — | C1-1 = 0.35 | 100° C./130° C. |
| 10 | | | | parts | |
| Composition | A9 = 10 parts | — | — | C1-1 = 0.35 | 100° C./130° C. |
| 11 | | | | parts | |
| Composition | A10 = 10 | — | — | C1-1 = 0.35 | 100° C./130° C. |
| 12 | parts | | | parts | |
| Composition | A11 = 10 | — | — | C1-1 = 0.35 | 100° C./130° C. |
| 13 | parts | | | parts | |
| Composition | A12 = 10 | — | — | C1-1 = 0.35 | 100° C./130° C. |
| 14 | parts | | | parts | |
| Composition | A13 = 10 | — | — | C1-1 = 0.35 | 100° C./130° C. |
| 15 | parts | | | parts | |
| Composition | A14 = 10 | — | — | C1-1 = 0.35 | 100° C./130° C. |
| 16 | parts | | | parts | |
| Composition | A15 = 10 | — | — | C1-1 = 0.35 | 100° C./130° C. |
| 17 | parts | | | parts | |
| Composition | A16 = 10 | — | — | C1-1 = 0.35 | 100° C./130° C. |
| 18 | parts | | | parts | |
| Composition | A17 = 10 | — | — | C1-1 = 0.35 | 100° C./130° C. |
| 19 | parts | | | parts | |
| Comparative | AX1 = 10 | — | — | C1-1 = 0.35 | 100° C./130° C. |
| Composition | parts | | | parts | |
| 1 | | | | | |
| Comparative | AX2 = 10 | — | — | C1-1 = 0.35 | 100° C./130° C. |
| Composition | parts | | | parts | |
| 2 | | | | | |

### <Resin (A)>

A1 to A17, AX1, AX2: Resins A1 to A17, resin AX1, and resin AX2

### <Salt (I)>

I-2401: salt represented by formula (I-2401)

<Acid generating agent (B)>

B1-25: Salt represented by formula (B1-25); synthesized by the method disclosed in Japanese Patent Laid-Open No. 2011-126869

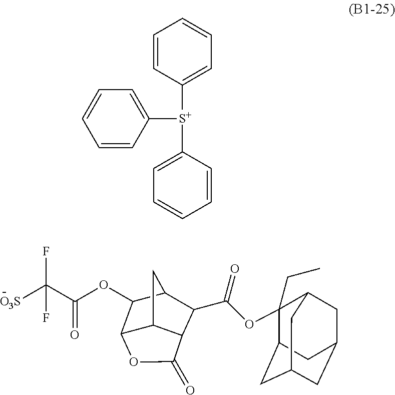

### <Quencher (C)>

C1-1: synthesized by method disclosed in Japanese Patent Laid-Open No. 2011-39502

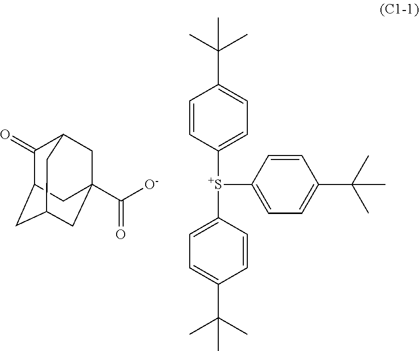 [SMILES: O=C1C2CC3CC1CC(C(=O)[O-])(C3)C2 ; CC(C)(C)c1ccc([S+](c2ccc(C(C)(C)C)cc2)c2ccc(C(C)(C)C)cc2)cc1]

### <Solvent (E)>

Propylene glycol monomethyl ether acetate 250 parts

Propylene glycol monomethyl ether 250 parts

γ-butyrolactone 5 parts

### (Electron Beam Exposure Evaluation of Resist Composition: Alkaline Development)

A 6-inch silicon wafer was treated with hexamethyldisilazane on a direct hot plate at 90° C. for 60 seconds. The resist composition was spin-coated onto this silicon wafer such that the film thickness of the composition layer was 0.04 μm. Then, the wafer was pre-baked on the direct hot plate for 60 seconds at the temperature shown in the “PB” column of Table 2 to form a composition layer. An electron beam drawing machine (ELS-F125 125 keV, manufactured by ELIONIX INC.) was used to perform direct drawing for the composition layer formed on the wafer by gradually changing the exposure dose so as to provide a contact hole pattern (hole pitch 40 nm/hole diameter 17 nm).

After exposure, a post-exposure bake was performed for 60 seconds at the temperature shown in the “PEB” column of Table 2 on a hot plate, followed by paddle development for 60 seconds with a 2.38 mass % tetramethylammonium hydroxide aqueous solution to obtain a resist pattern.

In the resist pattern obtained after development, the exposure dose that resulted in a formed hole diameter of 17 nm was defined as the effective sensitivity.

### <CD Uniformity (CDU) Evaluation>

At the effective sensitivity, the hole diameter of the pattern formed with a hole diameter of 17 nm was measured 24 times per hole, and its average value was defined as an average hole diameter of one hole. Those obtained by measuring the average hole diameter of the pattern formed with a hole diameter of 17 nm at 400 locations within the same wafer were used as a population to determine the standard deviation.

The results are shown in Table 3. The values in the table indicate the standard deviation (nm).

TABLE 3

| | Resist composition | CDU |
|---|---|---|
| Example 35 | Composition 1 | 2.28 |
| Example 36 | Composition 2 | 2.32 |
| Example 37 | Composition 3 | 2.40 |
| Example 38 | Composition 4 | 2.17 |
| Example 39 | Composition 5 | 2.12 |
| Example 40 | Composition 6 | 2.08 |
| Example 41 | Composition 7 | 2.10 |
| Example 42 | Composition 8 | 2.03 |
| Example 43 | Composition 9 | 2.04 |
| Example 44 | Composition 10 | 2.07 |
| Example 45 | Composition 11 | 2.00 |
| Example 46 | Composition 12 | 2.01 |
| Example 47 | Composition 13 | 2.10 |
| Example 48 | Composition 14 | 2.01 |
| Example 49 | Composition 15 | 2.02 |
| Example 50 | Composition 16 | 1.95 |
| Example 51 | Composition 17 | 2.10 |
| Example 52 | Composition 18 | 1.96 |
| Example 53 | Composition 19 | 1.97 |
| Comparative | Comparative | 2.58 |
| Example 1 | Composition 1 | |
| Comparative | Comparative | 2.49 |
| Example 2 | Composition 2 | |

Compared to Comparative Compositions 1 and 2, the compositions 1 to 19 had smaller standard deviations, and had better CD uniformity (CDU) evaluation.

### INDUSTRIAL APPLICABILITY

Since the present invention can produce a resist pattern having good CD uniformity (CDU), it is useful for semiconductor microfabrication.

1. A resist composition comprising a salt represented by formula (I) or a resin that includes a structural unit represented by formula (IP): 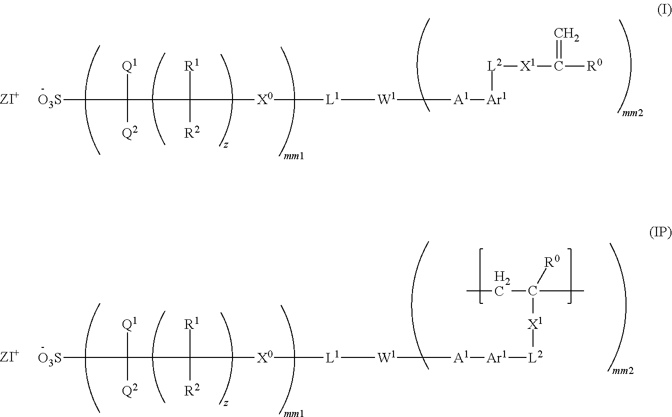
wherein in formula (I) and formula (IP),
Q¹, Q², R¹, and R² each independently represent a hydrogen atom, a fluorine atom, a perfluoroalkyl group having 1 to 6 carbon atoms, or an alkyl group having 1 to 6 carbon atoms;
z represents an integer of 0 to 6, and when z is 2 or larger, a plurality of R¹ and R² may be the same or different from each other;
X⁰ represents a single bond, —O—, —CO—, —S—, —SO—, —SO₂—, —NR⁷—, or a combined group of these groups;
R⁷ represents a hydrogen atom or an alkyl group having 1 to 6 carbon atoms;
mm1 represents 0 or 1;
L¹ and L² each independently represent a single bond, or a hydrocarbon group having 1 to 28 carbon atoms and optionally having a substituent, and —CH₂— contained in the hydrocarbon group is optionally replaced with —O—, —S—, —SO—, —SO₂—, —NR⁷—, or —CO—;
W¹ represents an alicyclic hydrocarbon group having 3 to 36 carbon atoms and optionally having a substituent, and —CH₂— contained in the alicyclic hydrocarbon group is optionally replaced with —O—, —CO—, —S—, —SO—, —SO₂—, or —NR⁷—;
A¹ represents a hydrocarbon group having 1 to 20 carbon atoms and optionally having a substituent, and —CH₂— contained in the hydrocarbon group is optionally replaced with —O—, —S—, —SO—, —SO₂—, —NR⁷—, or —CO—;
Ar¹ represents an aromatic hydrocarbon group having 6 to 18 carbon atoms having an iodine atom, and the aromatic hydrocarbon group optionally has a substituent other than an iodine atom;
X¹ represents a single bond, —O—, —CO—, —NR⁷—, —S—, —SO—, —SO₂—, a phenylene group optionally having a substituent, or a combined group of these groups;
R⁰ represents a hydrogen atom, a halogen atom, or an alkyl group having 1 to 6 carbon atoms and optionally having a halogen atom;
mm2 represents an integer of 1 to 5, and when mm2 is 2 or larger, a plurality of groups in parentheses may be the same or different from each other; and
ZI⁺ represents an organic cation.

2. The resist composition according to claim 1, wherein L¹ is a single bond, a chain hydrocarbon group having 1 to 10 carbon atoms and optionally having a substituent, where —CH₂— contained in the chain hydrocarbon group is optionally replaced with —O—, —CO—, —S—, —SO—, —SO₂—, or —NR⁷—, a cyclic hydrocarbon group having 3 to 18 carbon atoms and optionally having a substituent, where —CH₂— contained in the cyclic hydrocarbon group is optionally replaced with —O—, —CO—, —S—, —SO—, —SO₂—, or —NR⁷—, or a combined group of these groups.

3. The resist composition according to claim 1, wherein W¹ is an alicyclic hydrocarbon group having 5 to 18 carbon atoms and optionally having a substituent.

4. The resist composition according to claim 1,
wherein A¹ is ***—X⁰¹-L⁰¹- or ***-L⁰¹-X⁰¹—;
X⁰¹ represents —O—, —S—, —SO—, —SO₂—, —NR⁷—, or —CO—;
L⁰¹ represents a single bond, or a hydrocarbon group having 1 to 18 carbon atoms, where the hydrocarbon group optionally has a substituent, and —CH₂— contained in the hydrocarbon group is optionally replaced with —O—, —S—, —SO—, —SO₂—, —NR⁷—, or —CO—;
*** represents a bonding site to W¹.

5. The resist composition according to claim 4, wherein X⁰¹ is —O— or —CO—, and
L⁰¹ is a single bond or an alkanediyl group having 1 to 6 carbon atoms, where —CH₂— contained in the alkanediyl group is optionally replaced with —O— or —CO—.

6. The resist composition according to claim 1, wherein Ar¹ is an aromatic hydrocarbon group having 6 to 10 carbon atoms and having an iodine atom.

7. The resist composition according to claim 1, wherein X¹ is a single bond or a group represented by any one of formula (X¹-1) to (X¹-10): 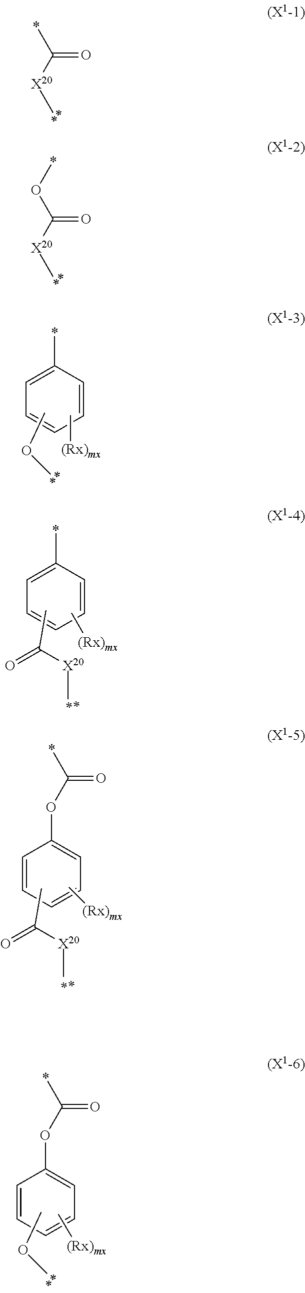 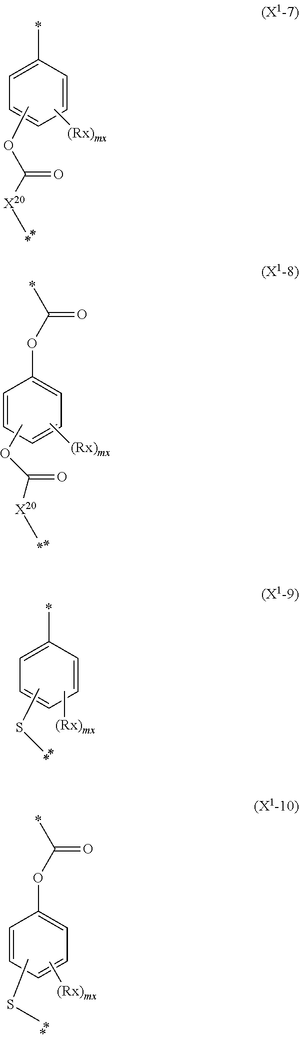
wherein in formula (X¹-1) to formula (X¹-10),
* and ** are bonding sites, and * represents a bonding site to a carbon atom bonded to R⁰;
R^{x} represents a halogen atom, a hydroxy group, a fluorinated alkyl group having 1 to 6 carbon atoms, an alkyl group having 1 to 18 carbon atoms, or an alkoxy group having 1 to 6 carbon atoms;
mx represents an integer of 0 to 4; when mx is 2 or more, a plurality of Rx are same or different from each other;
X²⁰ represents —O— or —NR⁷—; and
R⁷ represents a hydrogen atom or an alkyl group having 1 to 6 carbon atoms.

8. The resist composition according to claim 1,
wherein the resist composition further includes a resin that includes a structural unit represented by formula (a2-A); or
the resin that includes a structural unit represented by formula (IP) further includes a structural unit represented by formula (a2-A): 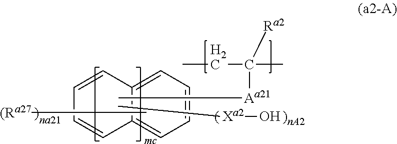
wherein in the formula (a2-A)
R^{a2} represents a hydrogen atom, a halogen atom, or an alkyl group having 1 to 6 carbon atoms and optionally having a halogen atom;
R^{a27} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group or a methacryloyloxy group;
A^{a21} represents a single bond, or an alkanediyl group having 1 to 12 carbon atoms, and —CH₂— contained in the alkanediyl group is optionally replaced with —O—, —CO— or —NR^{a28}—;
R^{a28} represents a hydrogen atom, or an alkyl group having 1 to 6 carbon atoms;
X^{a2} represents a single bond or a carbonyl group;
nA2 represents an integer of 1 to 5, and when nA2 is 2 or larger, a plurality of groups in parentheses are the same or different from each other;
na21 represents an integer of 0 to 4, and when na21 is 2 or larger, a plurality of R^{a27} may be the same or different from each other; and
mc represents an integer of 0 to 2.

9. The resist composition according to claim 1,
wherein the resist composition further includes a resin including a structural unit (a1) having an acid labile group; or
the resin that includes a structural unit represented by formula (IP) further includes a structural unit (a1) having an acid labile group, and
the structural unit (a1) having an acid labile group includes at least one selected from the group consisting of a structural unit represented by formula (a1-0), a structural unit represented by formula (a1-1), a structural unit represented by formula (a1-2), a structural unit represented by formula (a1-4), a structural unit represented by formula (a1-5), and a structural unit represented by formula (a1-6): 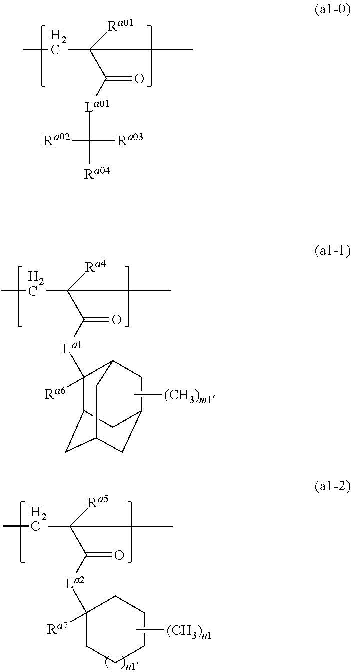
wherein in formula (a1-0), formula (a1-1), and formula (a1-2),
L^{a01}, L^{a1} and L^{a2} each independently represent —O— or *—O—(CH₂)ₖ₁—CO—O—, k1 represents an integer of 1 to 7, and * represents a bonding site to —CO—;
R^{a01}, R^{a4} and R^{a8} each independently represent a hydrogen atom, a halogen atom, or an alkyl group having 1 to 6 carbon atoms and optionally having a halogen atom;
R^{a02}, R^{a03} and R^{a04} each independently represent an alkyl group having 1 to 8 carbon atoms, an alicyclic hydrocarbon group having 3 to 18 carbon atoms, an aromatic hydrocarbon group having 6 to 18 carbon atoms, or a combined group of these groups, and the alkyl group, the alicyclic hydrocarbon group and the aromatic hydrocarbon group optionally have a halogen atom;
R^{a6} and R^{a7} each independently represent an alkyl group having 1 to 8 carbon atoms, an alkenyl group having 2 to 8 carbon atoms, an alicyclic hydrocarbon group having 3 to 18 carbon atoms, an aromatic hydrocarbon group having 6 to 18 carbon atoms, or a group formed by combining these groups, and the alkyl group, the alkenyl group, the alicyclic hydrocarbon group, and the aromatic hydrocarbon group optionally have a halogen atom;
m1′ represents an integer of 0 to 14;
n1 represents an integer of 0 to 14; and
n1′ represents an integer of 0 to 3; 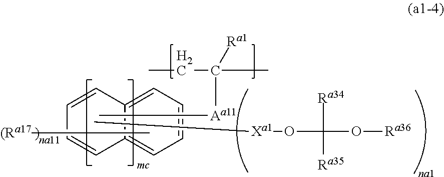
wherein in formula (a1-4),
R^{a1} represents a hydrogen atom, a halogen atom, or an alkyl group having 1 to 6 carbon atoms and optionally having a halogen atom;
R^{a17} represents a halogen atom, a hydroxy group, a carboxy group, an alkyl group having 1 to 6 carbon atoms and optionally having a halogen atom, an alkoxy group having 1 to 6 carbon atoms, an alkoxyalkyl group having 2 to 12 carbon atoms, an alkoxyalkoxy group having 2 to 12 carbon atoms, an alkylcarbonyl group having 2 to 4 carbon atoms, an alkylcarbonyloxy group having 2 to 4 carbon atoms, an acryloyloxy group, or a methacryloyloxy group;
A^{a11} represents a single bond or an alkanediyl group having 1 to 12 carbon atoms, and —CH₂— contained in the alkanediyl group is optionally replaced with —O—, —CO— or —NR^{a18}—;
R^{a18} represents a hydrogen atom or an alkyl group having 1 to 6 carbon atoms;
X^{a1} represents a single bond or a carbonyl group;
R^{a34} and R^{a35} each independently represent a hydrogen atom or a hydrocarbon group having 1 to 12 carbon atoms, R^{a36} represents a hydrocarbon group having 1 to 20 carbon atoms, or R^{a34} represents a hydrogen atom or a hydrocarbon group having 1 to 12 carbon atoms, R^{a35} and R^{a36} are bonded to each other to form a divalent heterocyclic group having 3 to 20 carbon atoms together with —C—O— to which they are bonded, and —CH₂— contained in the hydrocarbon group and the divalent heterocyclic group is optionally replaced with —O— or —S—;
na1 represents an integer of 1 to 5, and when na1 is 2 or larger, a plurality of groups in parentheses may be the same or different from each other;
na11 represents an integer of 0 to 4, and when na11 is 2 or larger, a plurality of R^{a17} may be the same or different from each other; and
mc represents an integer of 0 to 2; 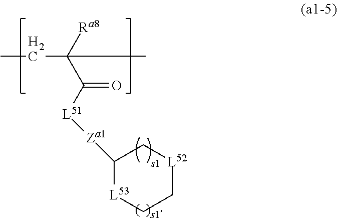
wherein in formula (a1-5),
R^{a8} represents an alkyl group having 1 to 6 carbon atoms and optionally having a halogen atom, a hydrogen atom or a halogen atom;
Z^{a1} represents a single bond or *—(CH₂)ₕ₃—CO-L⁵⁴-, h3 represents an integer of 1 to 4, and * represents a bonding site to L⁵¹;
L⁵¹, L⁵², L⁵³ and L⁵⁴ each independently represent —O— or —S—;
s1 represents an integer of 1 to 3;
s1′ represents an integer of 0 to 3; and 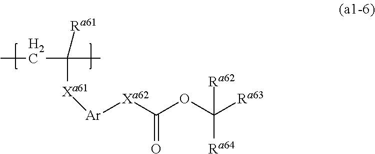
wherein in formula (a1-6),
R^{a61} represents a hydrogen atom, a halogen atom, or an alkyl group having 1 to 6 carbon atoms and optionally having a halogen atom;
R^{a62}, R^{a63} and R^{a64} each independently represent an alkyl group having 1 to 6 carbon atoms or a cyclic hydrocarbon group having 3 to 18 carbon atoms and optionally having a substituent, or R^{a62} and R^{a63} are bonded to each other to form a ring having 3 to 20 carbon atoms together with carbon atoms to which they are bonded, and R^{a64} represents an alkyl group having 1 to 6 carbon atoms or a cyclic hydrocarbon group having 3 to 18 carbon atoms and optionally having a substituent;
X^{a61} represents a single bond, —CO—O—*, or —CO—NR^{a65}—*, * represents a bonding site to Ar, and R^{a65} represents a hydrogen atom or an alkyl group having 1 to 6 carbon atoms;
X^{a62} represents a single bond, *—O-L^{a61}- or *—CO—O-L^{a62}-, * represents a bonding site to Ar, and L^{a61} and L^{a62} each independently represent an alkanediyl group having 1 to 4 carbon atoms; and
Ar represents an aromatic hydrocarbon group having 6 to 20 carbon atoms and optionally having a substituent.

10. The resist composition according to claim 1, further comprising a salt that generates an acid having a weaker acidity than an acid that is generated from the salt represented by the formula (I) or the resin that includes a structural unit represented by the formula (IP).

11. A method for producing a resist pattern, comprising:
(1) a step of applying the resist composition according to claim 1 onto a substrate;
(2) a step of drying the applied resist composition to form a composition layer;
(3) a step of exposing the composition layer;
(4) a step of heating the exposed composition layer; and
(5) a step of developing the composition layer after heating.

12. A salt represented by formula (I): 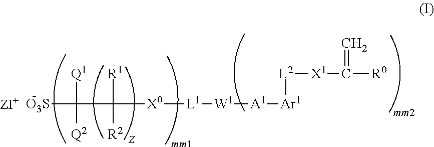
wherein in formula (I),
Q¹, Q², R¹, and R² each independently represent a hydrogen atom, a fluorine atom, a perfluoroalkyl group having 1 to 6 carbon atoms, or an alkyl group having 1 to 6 carbon atoms;
z represents an integer of 0 to 6, and when z is 2 or larger, a plurality of R¹ and R² may be the same or different from each other;
X⁰ represents a single bond, —O—, —CO—, —S—, —SO—, —SO₂—, —NR⁷—, or a combined group of these groups;
R⁷ represents a hydrogen atom or an alkyl group having 1 to 6 carbon atoms;
mm1 represents 0 or 1;
L¹ and L² each independently represent a single bond, or a hydrocarbon group having 1 to 28 carbon atoms and optionally having a substituent, and —CH₂— contained in the hydrocarbon group is optionally replaced with —O—, —S—, —SO—, —SO₂—, —NR⁷—, or —CO—;
W¹ represents an alicyclic hydrocarbon group having 3 to 36 carbon atoms and optionally having a substituent, and —CH₂— contained in the alicyclic hydrocarbon group is optionally replaced with —O—, —CO—, —S—, —SO—, —SO₂—, or —NR⁷—;
A¹ represents a hydrocarbon group having 1 to 20 carbon atoms and optionally having a substituent, and —CH₂— contained in the hydrocarbon group is optionally replaced with —O—, —S—, —SO—, —SO₂—, —NR⁷—, or —CO—;
Ar¹ represents an aromatic hydrocarbon group having 6 to 18 carbon atoms having an iodine atom, and the aromatic hydrocarbon group optionally has a substituent other than an iodine atom;
X¹ represents a single bond, —O—, —CO—, —NR⁷—, —S—, —SO—, —SO₂—, a phenylene group optionally having a substituent, or a combined group of these groups;
R⁰ represents a hydrogen atom, a halogen atom, or an alkyl group having 1 to 6 carbon atoms and optionally having a halogen atom;
mm2 represents an integer of 1 to 5, and when mm2 is 2 or larger, a plurality of groups in parentheses may be the same or different from each other; and
ZI⁺ represents an organic cation.

13. The salt according to claim 12, wherein L¹ is a single bond, a chain hydrocarbon group having 1 to 10 carbon atoms and optionally having a substituent, where —CH₂— contained in the chain hydrocarbon group is optionally replaced with —O—, —CO—, —S—, —SO—, —SO₂—, or —NR⁷—, a cyclic hydrocarbon group having 3 to 18 carbon atoms and optionally having a substituent, where —CH₂— contained in the cyclic hydrocarbon group is optionally replaced with —O—, —CO—, —S—, —SO—, —SO₂—, or —NR⁷—, or a combined group of these groups.

14. The salt according to claim 12, wherein W¹ is an alicyclic hydrocarbon group having 5 to 18 carbon atoms and optionally having a substituent.

15. The salt according to claim 12,
wherein A¹ is ***—X⁰¹-L⁰¹- or ***-L⁰¹-X⁰¹—;
X⁰¹ represents —O—, —S—, —SO—, —SO₂—, —NR⁷—, or —CO—;
L⁰¹ represents a single bond, or a hydrocarbon group having 1 to 18 carbon atoms, where the hydrocarbon group optionally has a substituent, and —CH₂— contained in the hydrocarbon group is optionally replaced with —O—, —S—, —SO—, —SO₂—, —NR⁷—, or —CO—;
*** represents a bonding site to W¹.

16. The salt according to claim 15, wherein X⁰¹ is —O— or —CO—, and
L⁰¹ is a single bond or an alkanediyl group having 1 to 6 carbon atoms, where —CH₂— contained in the alkanediyl group is optionally replaced with —O— or —CO—.

17. The salt according to claim 12, wherein Ar¹ is an aromatic hydrocarbon group having 6 to 10 carbon atoms and having an iodine atom.

18. The salt according to claim 12, wherein X¹ is a single bond or a group represented by any one of formulas (X¹-1) to (X¹-10): 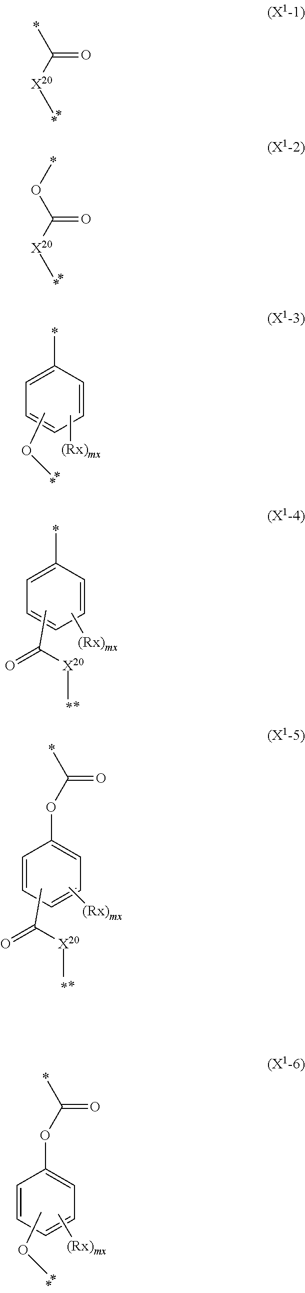 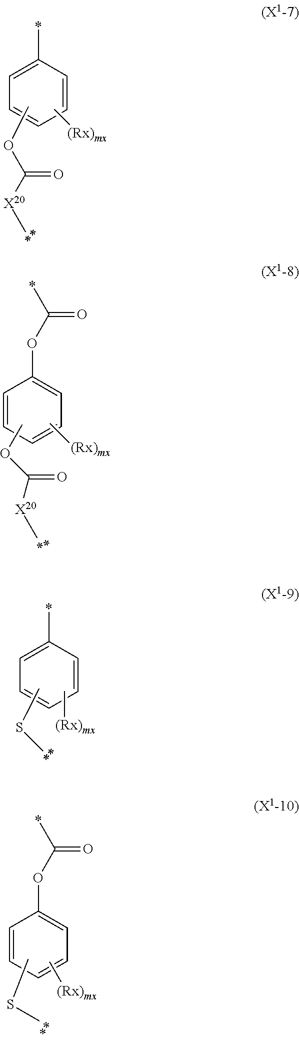
wherein in formula (X¹-1) to formula (X¹-10),
* and ** are bonding sites, and * represents a bonding site to a carbon atom bonded to R⁰;
R^{x} represents a halogen atom, a hydroxy group, a fluorinated alkyl group having 1 to 6 carbon atoms, an alkyl group having 1 to 18 carbon atoms, or an alkoxy group having 1 to 6 carbon atoms; and
mx represents an integer of 0 to 4; when mx is 2 or more, a plurality of Rx are the same or different from each other;
X²⁰ represents —O— or —NR⁷—; and
R⁷ represents a hydrogen atom or an alkyl group having 1 to 6 carbon atoms.

19. A resin comprising a structural unit represented by formula (IP): 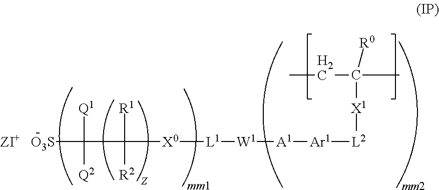
wherein in formula (IP),
Q¹, Q², R¹, and R² each independently represent a hydrogen atom, a fluorine atom, a perfluoroalkyl group having 1 to 6 carbon atoms, or an alkyl group having 1 to 6 carbon atoms;
z represents an integer of 0 to 6, and when z is 2 or larger, a plurality of R¹ and R² may be the same or different from each other;
X⁰ represents a single bond, —O—, —CO—, —S—, —SO—, —SO₂—, —NR⁷—, or a combined group of these groups;
R⁷ represents a hydrogen atom or an alkyl group having 1 to 6 carbon atoms;
mm1 represents 0 or 1;
L¹ and L² each independently represent a single bond, or a hydrocarbon group having 1 to 28 carbon atoms and optionally having a substituent, and —CH₂— contained in the hydrocarbon group is optionally replaced with —O—, —S—, —SO—, —SO₂—, —NR⁷—, or —CO—;
W¹ represents an alicyclic hydrocarbon group having 3 to 36 carbon atoms and optionally having a substituent, and —CH₂— contained in the alicyclic hydrocarbon group is optionally replaced with —O—, —CO—, —S—, —SO—, —SO₂—, or —NR⁷—;
A¹ represents a hydrocarbon group having 1 to 20 carbon atoms and optionally having a substituent, and —CH₂— contained in the hydrocarbon group is optionally replaced with —O—, —S—, —SO—, —SO₂—, —NR⁷—, or —CO—;
Ar¹ represents an aromatic hydrocarbon group having 6 to 18 carbon atoms having an iodine atom, and the aromatic hydrocarbon group optionally has a substituent other than an iodine atom;
X¹ represents a single bond, —O—, —CO—, —NR⁷—, —S—, —SO—, —SO₂—, a phenylene group optionally having a substituent, or a combined group of these groups;
R⁰ represents a hydrogen atom, a halogen atom, or an alkyl group having 1 to 6 carbon atoms and optionally having a halogen atom;
mm2 represents an integer of 1 to 5, and when mm2 is 2 or larger, a plurality of groups in parentheses may be the same or different from each other; and
ZI⁺ represents an organic cation.
